(19) United States
(12) Patent Application Publication
(10) Pub. No.: US 2024/0279749 A1
(43) Pub. Date: Aug. 22, 2024

(54) HIGH THROUGHPUT NUCLEIC ACID TESTING OF BIOLOGICAL SAMPLES

(71) Applicant: ABBOTT LABORATORIES, Abbott Aark, IL (US)

(72) Inventors: Matthew Frankel, Libertyville, IL (US); Patrick Fritchie, Southlake, TX (US); Gregg Williams, Villa Park, IL (US); Bradley Weston, Dallas, TX (US)

(73) Assignee: ABBOTT LABORATORIES, Abbott Aark, IL (US)

(21) Appl. No.: 18/557,631

(22) PCT Filed: Apr. 29, 2022
(86) PCT No.: PCT/US2022/027067

§ 371 Date: Oct. 27, 2023

Related U.S. Application Data
(60) Provisional application No. 63/181,799, filed on Apr. 29, 2021.
(60) Provisional application No. 63/181,822, filed on Apr. 29, 2021.
(60) Provisional application No. 63/181,874, filed on Apr. 29, 2021.
(60) Provisional application No. 63/181,880, filed on Apr. 29, 2021.
(60) Provisional application No. 63/302,939, filed on Jan. 25, 2022.
(60) Provisional application No. 63/302,957, filed on Jan. 25, 2022.
(60) Provisional application No. 63/302,959, filed on Jan. 25, 2022.
(60) Provisional application No. 63/302,982, filed on Jan. 25, 2022.

(51) Int. Cl. C12Q 1/70; B01L 3/00; C12Q 1/6806; C12Q 1/6848; G01N 35/00; G01N 35/02; G01N 35/04; G01N 35/10

(52) U.S. Cl. CPC C12Q 1/70; B01L 3/502; C12Q 1/6806; C12Q 1/6848; G01N 35/0099; G01N 35/025; B01L 2200/0647; B01L 2200/141; B01L 2300/0829; G01N 2035/0406; G01N 2035/041; G01N 2035/0446; G01N 2035/103

(57) ABSTRACT

The presently disclosed subject matter relates to methods for rapid, sensitive, and high-throughput nucleic acid testing of biological samples, e.g., blood, serum, or plasma samples from donors, as well as systems capable of performing such high-throughput nucleic acid testing.

### CROSS-REFERENCE TO RELATED APPLICATIONS

This application is a U.S. National Stage Patent Application under 35 U.S.C. § 371 of International Patent Application No. PCT/US2022/027067, filed Apr. 29, 2022, which claims priority to U.S. provisional application No. 63/302,957, filed Jan. 25, 2022, and U.S. provisional application No. 63/302,959, filed Jan. 25, 2022, and U.S. provisional application No. 63/302,982, filed Jan. 25, 2022, and U.S. provisional application No. 63/302,939, filed Jan. 25, 2022, and U.S. provisional application No. 63/181,799, filed Apr. 29, 2021, and U.S. provisional application No. 63/181,822, filed Apr. 29, 2021, and U.S. provisional application No. 63/181,874, filed Apr. 29, 2021, and U.S. provisional application No. 63/181,880, filed Apr. 29, 2021, the entire contents of each of which are hereby incorporated by reference.

### SEQUENCE LISTING

The instant application contains a Sequence Listing which has been submitted in ASCII format electronically and is hereby incorporated by reference in its entirety. Said ASCII copy, created on May 6, 2024, is named 003168_4079.txt and is 32,079 bytes in size. The Sequence Listing, electronically filed herewith, does not extend beyond the scope of the specification and thus does not contain new matter.

### BACKGROUND

### Field of the Disclosed Subject Matter

The presently disclosed subject matter relates to methods for rapid, sensitive, and high-throughput nucleic acid testing of biological samples, e.g., blood, serum, or plasma samples from donors and/or patients, as well as systems capable of performing such high-throughput nucleic acid testing.

### Description of Related Art

Screening of donated blood and plasma has an essential role in safeguarding supply of life-saving whole blood, plasma, platelets, red blood cells and blood products manufactured from whole blood or plasma. Not surprisingly, screening of donated blood and plasma is often highly regulated to ensure that the blood and plasma are free of pathogens and infectious agents. Governmental agencies and other accredited entities have provided detailed guidance on the processes for whole blood and plasma screening for their respective jurisdictions. For example, the U.S. Food and Drug Administration provides guidance in the U.S. for appropriate donated blood screening processes. Other agencies, such as the World Health Organization (WHO) or country specific health agencies, also provide guidance on donated blood screening. Although there are common approaches to screening of donated blood in different jurisdictions, there are no universal requirements and thus screening guidance differs from jurisdiction to jurisdiction.

Detection of pathogens and infectious agents in the donated blood supply currently often involves serologic testing as well as nucleic acid testing (“NAT”). Serologic testing refers to the testing by immunoassays to detect antigens from the pathogens or infectious agents and/or the presence of antibodies a donor has raised against pathogens or infectious agents. In contrast, NAT refers to the detection of the genetic material (e.g., DNA and/or RNA) associated with a pathogen or infectious agent. In an exemplary workflow for screening donated blood for transfusion, blood is collected from a donor into a blood bag and into multiple sample collection tubes at a collection site. The donor will also answer a survey requesting information relevant to the donated blood. The blood bag is transported to a blood collection facility for further processing and storage. The collection tubes are transported to a blood screening laboratory to subject the samples to testing for pathogens or infectious diseases and for blood grouping and typing. A sample may be subjected to immunoassay testing for, e.g., human immunodeficiency virus (HIV), hepatitis B virus (HBV)-specific antigens, e.g., hepatitis B surface antigen (HBsAg), human T-cell lymphotropic virus (HTLV), Chagas, cytomegalovirus (CMV), Syphilis, anti-virus antibodies, such as, anti-HBc, and anti-HCV antibodies. The sample may also be subjected to NAT for, e.g., HIV, HCV, HBV, Babesia, Zika and West Nile Virus. Commonly, an informatics system tracks test results for each of the tests for each sample. If a test result for an analyte is positive, the donated blood is quarantined, and the donor is notified of the screening result. If all tests are negative for a specific sample, the donated blood is released for clinical use.

Similarly, in an exemplary workflow for screening donated plasma, plasma is collected from a donor such as through plasmapheresis. During this process, whole blood is removed, plasma is collected, and the remaining blood components are returned to the donor. Plasma screening may include the above-described immunoassay and NAT and may additionally include NAT for Parvovirus B19 and hepatitis A virus (HAV). Although the above described tests are commonly used for screening, different tests for new analytes or targets of interest may be used in various jurisdictions or as new outbreaks and infections develop.

Many jurisdictions require NAT due to its higher sensitivity in detection of low levels of pathogens which levels may be below the limits of detection in a serological assay. The jurisdictions often rely on an overlapping testing strategy whereby a low-cost, high-throughput serology assays screen donated blood to see if the donors have ever (or currently) are infected with a transfusion-transmitted pathogen or infectious agent, and then a more direct, time-consuming process for NAT to see if they are currently infected.

Current NAT-based screening, however, involves test times that can be hours longer than serology. Due to its relatively high expense and long turn-around time, NAT-based screening of donated blood and plasma creates a significant bottleneck and cost in screening of whole blood and plasma. The lag between time of donation of whole blood or plasma and time of release of the donated whole blood or plasma becomes especially pertinent in times of national/global health emergencies. In addition, a longer release time of whole blood reduces the shelf-life of the whole blood as well as parts thereof, such as, platelets and red blood cells that may be fractionated from the whole blood. Optimization and implementation of work-flow strategies have attempted to blunt the impact of the large time-investment on NAT-based screening. These techniques include pooling and work force scheduling to ensure that efficiencies can be gleaned despite the slower complex cadence of current NAT-based screening. But each of these workflow strategies create issues of their own. Work force scheduling for NAT-based screening can be expensive. With regard to pooling specifically, complex sample pooling strategies have been developed to facilitate higher throughput. Further, for traditional pooling, additional time and equipment is required to pool and there is added time and cost associated with deconstructing pooled samples if a pathogen or infectious agent is detected in a pooled sample. There is also a high risk of errors being made as a result of poor quality procedures during the preparation of the pool, when recording individual samples in each pool and when subsequently re-screening of pooled samples to identify individual samples positive for the tested pathogen or infectious agent. These pooling drawbacks are exacerbated in certain contexts, e.g., at plasma centers, where extensive pooling has become an industry norm and where significant pool deconstruction and rescreening may be necessary.

Moreover, despite serologic screening's relative low cost and rapid time to result, the personnel and infrastructure costs associated with running parallel screening regimes are a burden and present additional logistical challenges, particularly in low-resource locales. The cost and time-consuming nature of the current parallel screening regimes has led to the implementation of a variety of resource-intensive accommodations. In many instances, NAT-based screening is centralized to take advantage of economies of scale. Such accommodations, however, are not without their own drawbacks, including the cost and time associated with transporting samples to distant central screening labs that are distant from blood collection, processing and storage facilities, thus adding time to the release process.

Thus, there is a need for NAT that can be performed with higher throughput, such as, NAT that requires less time than the currently available NAT for whole blood and plasma screening. There is also a need for a whole blood and plasma screening assay that can replace the current process requiring both serological and NAT. The present invention fulfills these and other needs.

### SUMMARY

The present disclosure provides methods of screening samples of donor blood for release of the donor blood or a material from the donor for clinical use.

The present disclosure provides methods of screening one or more samples of donor blood for release of the donor blood or a material from the donor for clinical use, comprising performing a nucleic acid analysis on the one or more samples of donor blood to detect a plurality of pathogens or infectious agents; wherein a determination of a predetermined level of nucleic acids derived from each of the plurality of pathogens or infectious agents based on the nucleic acid analysis, is indicative of release of the donor blood or the donor material for clinical use, and wherein release of the donor blood for clinical use occurs in about 15 to about 60 minutes, e.g., about 20 to about 60 minutes, from initial aspiration of the one or more samples for performance of the nucleic acid analysis.

The present disclosure also provides methods of screening one or more samples of donor blood for release of the donor blood or a material from the donor for clinical use, comprising performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis, is indicative of release of the donor blood for clinical use, and wherein the method comprises screening a plurality of samples of donor blood for release of the donor blood or the donor material for clinical use and the determinations based on the nucleic acid analyses are performed within about 20 minutes to about 3.5 hours from initial aspiration of the first sample for performance of the nucleic acid analysis.

The present disclosure further provides methods of screening of one or more samples of donor blood for release of the donor blood or a material from the donor for clinical use, comprising performing a nucleic acid analysis on the one or more samples of donor blood to detect a plurality of pathogens or infectious agents; wherein a determination of a predetermined level of nucleic acids derived from each of the plurality of pathogens or infectious agents based on the nucleic acid analysis, is indicative of release of the donor blood or the donor material for clinical use, and wherein the nucleic acid analysis comprises a nucleic acid amplification reaction of about 8 minutes to about 20 minutes in duration.

The present disclosure provides methods of screening of one or more samples of donor blood for release of the donor blood or a material from the donor for clinical use, comprising performing a nucleic acid analysis on the one or more samples of donor blood to detect a plurality of pathogens or infectious agents; wherein a determination of a predetermined level of nucleic acids derived from each of the plurality of pathogens or infectious agents based on the nucleic acid analysis, is indicative of release of the donor blood or the donor material for clinical use, and wherein each determination of a predetermined level of nucleic acid from at least one of the plurality of pathogens or infectious agent is completed in about 20 to about 45 minutes from initial aspiration of the one or more samples for performance of the nucleic acid analysis.

The present disclosure provides methods of screening of one or more samples of donor blood for release of the donor blood or a material from the donor for clinical use, comprising performing a nucleic acid analysis on the one or more samples of donor blood to detect a plurality of pathogens or infectious agents; wherein a determination of a predetermined level of nucleic acids derived from each of the plurality of pathogens or infectious agents based on the nucleic acid analysis, is indicative of release of the donor blood or the donor material for clinical use, and wherein each determination of a predetermined level of nucleic acid from at least one of the plurality of pathogens or infectious agent has a time to result of about 20 to about 45 minutes.

The present disclosure provides methods of screening of one or more samples of donor blood for release of the donor blood or a material from the donor for clinical use, comprising performing nucleic acid analyses on the one or more samples of donor blood to detect a plurality of pathogens or infectious agents; wherein a determination of a predetermined level of nucleic acids derived from each of the plurality of pathogens or infectious agents based on the nucleic acid analyses, is indicative of release of the donor blood or the donor material for clinical use, and wherein at least about 70 results are obtained per hour per m³ of a volume occupied by the automated system.

The present disclosure provides methods of screening of one or more samples of donor blood for release of the donor blood or a material from the donor for clinical use, comprising performing nucleic acid analyses on the one or more samples of donor blood to detect a plurality of pathogens or infectious agents; wherein a determination of a predetermined level of nucleic acids derived from each of the plurality of pathogens or infectious agents based on the nucleic acid analysis, is indicative of release of the donor blood or the donor material for clinical use, and wherein at least about 140 results are obtained per hour per m² of a footprint of the automated system.

The present disclosure provides methods of screening of one or more samples of donor blood for release of the donor blood or a material from the donor for clinical use, comprising performing a nucleic acid analysis on the one or more samples of donor blood to detect a plurality of pathogens or infectious agents; wherein a determination of a predetermined level of nucleic acids derived from each of the plurality of pathogens or infectious agents based on the nucleic acid analysis, is indicative of release of the donor blood or the donor material for clinical use, and wherein, upon a determination of the presence of a nucleic acid derived from at least one of the plurality of pathogens or infectious agents in excess of the predetermined level in a pooled sample, the methods further comprising screening samples of individual donor blood or sub-pools thereof included in the pooled sample, comprising performing a nucleic acid analysis on the samples of donor blood to detect a plurality of pathogens or infectious agents; wherein a determination of a predetermined level of nucleic acids derived from each of the plurality of pathogens or infectious agents based on the nucleic acid analysis, is indicative of release of the donor blood or the donor material for clinical use.

The present disclosure provides methods for screening one or more samples to determine whether donor blood associated with the one or more samples is acceptable for transfusion comprising performing a nucleic acid analysis on the samples including a sample preparation step and an amplification step; determining a result from the nucleic acid analysis; and making a determination whether donor blood is acceptable for transfusion based at least in part on the nucleic acid analysis result wherein release of the donor blood or the donor material for clinical use occurs in about 15 to about 60 minutes, e.g., about 20 to about 60 minutes, from initial aspiration of the one or more samples for performance of the nucleic acid analysis.

The present disclosure also provides methods for screening a plurality of samples to determine whether donor blood associated with the samples is acceptable for transfusion comprising performing a nucleic acid analysis on the samples including a sample preparation step and an amplification step; determining a result from the nucleic acid analysis; and making a determination whether donor blood is acceptable for transfusion based at least in part on the nucleic acid analysis result wherein the determinations based on the nucleic acid analyses are performed within about 15 minutes to about 3.5 hours, e.g. about 20 minutes to about 3.5 hours, from initial aspiration of the first sample for performance of the nucleic acid analysis.

The present disclosure provides methods for screening a plurality of samples to determine whether donor blood associated with the samples is acceptable for transfusion comprising performing a nucleic acid analysis on the samples including a sample preparation step and an amplification step; determining a result from the nucleic acid analysis; and making a determination whether donor blood is acceptable for transfusion based at least in part on the nucleic acid analysis result wherein the nucleic acid analysis comprises a nucleic acid amplification reaction of about 1 minute to about 20 minutes, e.g., 8 minutes to about 20 minutes, in duration.

The present disclosure further provides methods for screening a plurality of samples to determine whether donor blood associated with the samples is acceptable for transfusion comprising performing a nucleic acid analysis on the samples including a sample preparation step and an amplification step; determining a result from the nucleic acid analysis; and making a determination whether donor blood is acceptable for transfusion based at least in part on the nucleic acid analysis result wherein each determination of a predetermined level of nucleic acid from at least one of the plurality of pathogens or infectious agent is completed in about 15 to about 45 minutes, e.g., about 20 to about 45 minutes, from initial aspiration of the sample for performance of the nucleic acid analysis.

The present disclosure provides methods for screening a plurality of samples to determine whether donor blood associated with the samples is acceptable for transfusion comprising performing a nucleic acid analysis on the samples including a sample preparation step and an amplification step determining a result from the nucleic acid analysis; and making a determination whether donor blood is acceptable for transfusion based at least in part on the nucleic acid analysis result wherein each determination of a predetermined level of nucleic acid from at least one of the plurality of pathogens or infectious has a time to result of about 15 to about 45 minutes, e.g., about 20 minutes to about 45 minutes.

The present disclosure provides methods for screening a plurality of samples to determine whether donor blood associated with the samples is acceptable for transfusion comprising performing a nucleic acid analysis on the samples including a sample preparation step and an amplification step; determining a result from each of the nucleic acid analyses of the samples; and making a determination whether donor blood is acceptable for transfusion based at least in part on the nucleic acid analysis results wherein at least about 70 results are obtained per hour per m³ of a volume occupied by the automated system, e.g., used to perform the method.

The present disclosure provides methods for screening a plurality of samples to determine whether donor blood associated with the samples is acceptable for transfusion comprising performing a nucleic acid analysis on the samples including a sample preparation step and an amplification step; determining a result from each of the nucleic acid analyses of the samples; and making a determination whether donor blood is acceptable for transfusion based at least in part on the nucleic acid analysis result wherein at least about 140 results are obtained per hour per m² of a footprint of the automated system, e.g., used to perform the method.

In certain embodiments, the nucleic acid analysis comprises a nucleic acid amplification reaction. In certain embodiments, the nucleic acid amplification reaction is an isothermal reaction. In certain embodiments, the nucleic acid analysis comprises optical detection of the presence of a nucleic acid derived from at least one of the plurality of pathogens or infectious agents. In certain embodiments, the nucleic acid analysis comprises digital detection of the presence of a nucleic acid derived from at least one of the plurality of pathogens or infectious agents. In certain embodiments, the nucleic acid analysis comprises contemporaneous contact of the sample with sample lysis buffer and a protease. In certain embodiments, the isothermal reaction is recombinase polymerase amplification. In certain embodiments, the isothermal reaction is a nicking enzyme amplification reaction. In certain embodiments, the nucleic acid analysis includes an amplification process, and the amplification process can include a nucleic acid amplification reaction. In certain embodiments, the nucleic acid amplification reaction of the amplification process includes an isothermal reaction. In certain embodiments, the nucleic acid amplification reaction of the amplification process includes recombinase polymerase amplification. In certain embodiments, the nucleic acid amplification reaction of the amplification process includes a nicking enzyme amplification reaction. In certain embodiments, the nucleic acid analysis includes a detection process. In certain embodiments, the detection process can include optical detection of the presence or absence of a nucleic acid derived from at least one of a plurality of pathogens or infectious agents. In certain embodiments, the detection process can include digital detection of the presence or absence of a nucleic acid derived from at least one of a plurality of pathogens or infectious agents. In certain embodiments, the nucleic acid analysis includes an amplification and detection process. In certain embodiments, the amplification and detection process can include a nucleic acid amplification reaction and optical detection of the presence or absence of a nucleic acid derived from at least one of a plurality of pathogens or infectious agents. In certain embodiments, the amplification and detection process can include a nucleic acid amplification reaction and digital detection of the presence or absence of a nucleic acid derived from at least one of a plurality of pathogens or infectious agents. In certain embodiments, the nucleic acid analysis includes a sample preparation process. In certain embodiments, the sample preparation process can include contemporaneous contact of a sample with sample lysis buffer and, optionally, with a protease, e.g., Proteinase K. In certain embodiments, the sample preparation process includes a lysis process, and the lysis process can include contemporaneous contact of a sample with sample lysis buffer and, optionally, with a protease. In certain embodiments, the sample preparation process includes a lysis process, pre-treatment lysis process, and/or onboard pooling process, as described further herein.

In certain embodiments, the plurality of pathogens or infectious agents are selected from the group consisting of: SARS-CoV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, Usutu virus and HEV. Additionally or alternatively, In certain embodiments, the plurality of pathogens or infectious agents can include one or more emerging pathogens, viruses, and/or agents.

In certain embodiments, the plurality of pathogens or infectious agents are HIV-1, HIV-2, HCV, and HBV. In certain embodiments, the plurality of pathogens or infectious agents are HIV-1 and HBV. In certain embodiments, the plurality of pathogens or infectious agents are HIV-2 and HCV. In certain embodiments, the plurality of pathogens or infectious agents are HIV-1, HIV-2, HCV, HBV, and WNV. In certain embodiments, the plurality of pathogens or infectious agents are Zika Virus and WNV. In certain embodiments, the plurality of pathogens or infectious agents are Chikungunya Virus and Dengue Virus. In certain embodiments, the plurality of pathogens or infectious agents are Zika Virus, WNV, Chikungunya Virus and Dengue Virus. In certain embodiments, the plurality of pathogens or infectious agents are Babesia and Malaria. In certain embodiments, the plurality of pathogens or infectious agents are Parvovirus B19 and HAV. In certain embodiments, the plurality of pathogens or infectious agents are HIV-1, HIV-2, HCV, HBV, WNV, and Zika Virus. In certain embodiments, the plurality of pathogens or infectious agents are HIV-1, HIV-2, HCV, HBV, WNV, Zika Virus, and Chikungunya Virus. In certain embodiments, the plurality of pathogens or infectious agents are HIV-1, HIV-2, HCV, HBV, WNV, Zika Virus, Chikungunya Virus, and Dengue Virus. In certain embodiments, the plurality of pathogens or infectious agents are HIV-1, HIV-2, HCV, HBV, WNV, Zika Virus, Chikungunya Virus, Dengue Virus, and Babesia. In certain embodiments, the plurality of pathogens or infectious agents are HIV-1, HIV-2, HCV, HBV, WNV, Zika Virus, Chikungunya Virus, Dengue Virus, Babesia, and Malaria. In certain embodiments, the plurality of pathogens or infectious agents are HIV-1, HIV-2, HCV, HBV, WNV, Zika Virus, Chikungunya Virus, Dengue Virus, Babesia, Malaria, and Parvovirus B19. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, and HAV. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV. In certain embodiments, the plurality of pathogens or infectious agents are HIV-1, HIV-2, HCV, HBV, and Parvovirus B19. In certain embodiments, the plurality of pathogens or infectious agents are HIV-1, HIV-2, HCV, HBV, Parvovirus B19, and HAV. In certain embodiments, the plurality of pathogens or infectious agents are HIV-1, HIV-2, HCV, HBV, and Babesia. In certain embodiments, the plurality of pathogens or infectious agents are HIV-1, HIV-2, HCV, HBV, and HAV. In certain embodiments, the plurality of pathogens or infectious agents are HIV-1, HIV-2, HCV, HBV, and HEV. In certain embodiments, the plurality of pathogens or infectious agents are HIV-1, HIV-2, HCV, HBV, and Zika Virus. In certain embodiments, the plurality of pathogens or infectious agents are HIV-1, HIV-2, HCV, HBV, Zika Virus, and Dengue Virus. In certain embodiments, the plurality of pathogens or infectious agents are HIV-1, HIV-2, HCV, HBV, Zika Virus, Dengue Virus, and Chikungunya Virus. In certain embodiments, the plurality of pathogens or infectious agents are HIV-1, HIV-2, HCV, HBV, Zika Virus, Dengue Virus, and WNV. In certain embodiments, the plurality of pathogens or infectious agents are HIV-1, HIV-2, HCV, HBV, Zika Virus, WNV, and Chikungunya Virus. In certain embodiments, the plurality of pathogens or infectious agents are HIV-1, HIV-2, HCV, HBV, Zika Virus, WNV, Dengue Virus, and Chikungunya Virus. In certain embodiments, the plurality of pathogens or infectious agents are HIV-1, HIV-2, HCV, HBV, and Malaria. In certain embodiments, the plurality of pathogens or infectious agents are HIV-1, HIV-2, HCV, HBV, Malaria, and Babesia. In certain embodiments, the plurality of pathogens or infectious agents are HIV-1, HIV-2, HCV, HBV, and Dengue Virus. In certain embodiments, the plurality of pathogens or infectious agents are HIV-1, HIV-2, HCV, HBV, Dengue Virus, and Chikungunya Virus. In certain embodiments, the plurality of pathogens or infectious agents are HIV-1, HIV-2, HCV, HBV, Dengue Virus, WNV, and Chikungunya Virus. In certain embodiments, the plurality of pathogens or infectious agents are HIV-1, HIV-2, HCV, HBV, and Chikungunya Virus. In certain embodiments, the plurality of pathogens or infectious agents are HIV-1, HIV-2, HCV, HBV, Chikungunya Virus, and Zika Virus. In certain embodiments, the plurality of pathogens or infectious agents are HIV-1, HIV-2, HCV, HBV, Chikungunya Virus, Zika Virus, and WNV. In certain embodiments, the plurality of pathogens or infectious agents are HIV-1, HIV-2, HCV, HBV, WNV, and Dengue Virus. In certain embodiments, the plurality of pathogens or infectious agents are HIV-1, HIV-2, HCV, HBV, Chikungunya Virus, Zika Virus, and WNV.

In certain embodiments, the plurality of pathogens or infectious agents are HIV-1, HIV-2, HCV, and HBV; and the nucleic acid analysis comprises multiplex analysis of HIV-1 and HIV-2. In certain embodiments, the plurality of pathogens or infectious agents are HIV-1, HIV-2, HCV, and HBV; and the nucleic acid analysis comprises multiplex analysis of HIV-1, HIV-2, and HCV. In certain embodiments, the plurality of pathogens or infectious agents are HIV-1, HIV-2, HCV, and HBV; and the nucleic acid analysis comprises multiplex analysis of HIV-1, HIV-2, and HBV. In certain embodiments, the plurality of pathogens or infectious agents are HIV-1, HIV-2, HCV, and HBV; and the nucleic acid analysis comprises multiplex analysis of HCV, and HBV. In certain embodiments, the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV. In certain embodiments, the plurality of pathogens or infectious agents are HIV-1, HIV-2, HCV, and HBV; and the nucleic acid analysis comprises multiplex analysis of HIV-1, HIV-2, HCV, and HBV.

In certain embodiments, the plurality of pathogens or infectious agents comprise Zika Virus, WNV, Chikungunya Virus and Dengue Virus; the nucleic acid analysis comprises multiplex analysis of Zika Virus and WNV. In certain embodiments, the plurality of pathogens or infectious agents comprise Zika Virus, WNV, Chikungunya Virus and Dengue Virus; the nucleic acid analysis comprises multiplex analysis of Chikungunya Virus and Dengue Virus. In certain embodiments, the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus. In certain embodiments, the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus. In certain embodiments, the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV. In certain embodiments, the plurality of pathogens or infectious agents comprise Babesia and Malaria; and the nucleic acid analysis comprises multiplex analysis of Babesia and Malaria. In certain embodiments, the plurality of pathogens or infectious agents comprise Parvovirus B19 and HAV; and the nucleic acid analysis comprises multiplex analysis of Parvovirus B19 and HAV.

In certain embodiments, the nucleic acid analysis comprises contacting the sample with CuTi-coated microparticles. In certain embodiments, the nucleic acid analysis comprises contacting the sample with a plurality of microparticles and translation of the microparticles on a surface via magnetic force. In certain embodiments, the nucleic acid analysis comprises purification of nucleic acid from the sample of donor blood; division of the purified nucleic acid into a plurality of fractions; and at least one fraction is reserved for further screening. In certain embodiments, the nucleic acid analysis comprises a sample preparation process, and the sample preparation process can include contacting the sample with CuTi-coated microparticles. For example and not limitation, the sample preparation process can include a lysis process, and the lysis process can include contacting the sample with CuTi-coated microparticles. In certain embodiments, the sample preparation process can include a wash process. For example and not limitation, the wash process can include translation of the microparticles on a surface via magnetic force. In certain embodiments, the sample preparation process can be performed in a sample preparation area. In certain embodiments, the sample preparation area can include a sample transport, e.g., a sample preparation carousel, and a wash and elution system. In certain embodiments, the sample preparation area can include a particle transfer mechanism and the particle transfer mechanism can transfer CuTi-coated microparticles from the sample transport, e.g., sample preparation carousel to the wash and elution system. For example, the particle transfer mechanism can be magnetic force, e.g., via a magnetic tip, to transfer CuTi-coated microparticles.

In certain embodiments, the plurality of pathogens or infectious agents and predetermined levels are selected from the following: SARS-CoV-2 (COVID-19) at a predetermined level of at least 1-50 copies/mL; HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; CMV at a predetermined level of at least 10-50 IU/mL; Parvovirus B19 at a predetermined level of at least 1-40 IU/mL; HAV at a predetermined level of at least 1-10 IU/mL; Chlamydia at a predetermined level of at least 100-500 copies/mL; Gonorrhea at a predetermined level of at least 100-500 copies/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Influenza at a predetermined level of at least 10-500 copies/mL; Babesia at a predetermined level of at least 1-20 copies/mL; Malaria at a predetermined level of at least 1-50 copies/mL; and HEV at a predetermined level of at least 1-20 IU/mL.

In certain embodiments, the plurality of pathogens or infectious agents are HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, HBV, and WNV; and the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV. In certain embodiments, the plurality of pathogens or infectious agents are Zika Virus and WNV; and the predetermined levels are: at least 1-50 copies/mL of Zika Virus; and at least 1-50 copies/mL of WNV. In certain embodiments, the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus. In certain embodiments, the plurality of pathogens or infectious agents are Zika Virus, WNV, Chikungunya Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the plurality of pathogens or infectious agents are Babesia and Malaria; and wherein the predetermined levels are at least 1-20 copies/mL of Babesia; and at least 1-50 copies/mL of Malaria. In certain embodiments, the plurality of pathogens or infectious agents are Parvovirus B19 and HAV; and the predetermined levels are at least 1-40 IU/mL of Parvovirus B19; and at least 1-10 IU/mL of HAV. In certain embodiments, the plurality of pathogens or infectious agents are HIV-1, HIV-2, HCV, HBV, WNV, and Zika Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus. In certain embodiments, the plurality of pathogens or infectious agents are HIV-1, HIV-2, HCV, HBV, WNV, Zika Virus, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus. In certain embodiments, the plurality of pathogens or infectious agents are HIV-1, HIV-2, HCV, HBV, WNV, Zika Virus, Chikungunya Virus, and Dengue Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

In certain embodiments, the plurality of pathogens or infectious agents are HIV-1, HIV-2, HCV, HBV, WNV, Zika Virus, Chikungunya Virus, Dengue Virus, and Babesia; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia. In certain embodiments, the plurality of pathogens or infectious agents are HIV-1, HIV-2, HCV, HBV, WNV, Zika Virus, Chikungunya Virus, Dengue Virus, Babesia, and Malaria; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria. In certain embodiments, the plurality of pathogens or infectious agents are HIV-1, HIV-2, HCV, HBV, WNV, Zika Virus, Chikungunya Virus, Dengue Virus, Babesia, Malaria, and Parvovirus B19; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; at least 1-50 copies/mL of Malaria; and at least 1-40 IU/mL of Parvovirus B19. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; at least 1-50 copies/mL of Malaria; at least 1-40 IU/mL of Parvovirus B19; and at least 1-10 IU/mL of HAV. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; at least 1-50 copies/mL of Malaria; at least 1-40 IU/mL of Parvovirus B19; at least 1-10 IU/mL of HAV; and at least 1-20 IU/mL of HEV.

In certain embodiments, the plurality of pathogens or infectious agents are HIV-1, HIV-2, HCV, HBV, and Parvovirus B19; and the predetermined levels are at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19. In certain embodiments, the plurality of pathogens or infectious agents are HIV-1, HIV-2, HCV, HBV, Parvovirus B19, and HAV; and the predetermined levels are at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV. In certain embodiments, the plurality of pathogens or infectious agents are HIV-1, HIV-2, HCV, HBV, and Babesia; and the predetermined levels are at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia. In certain embodiments, the plurality of pathogens or infectious agents are HIV-1, HIV-2, HCV, HBV, and HAV; and the predetermined levels are at least 1-50 copies/mL of HIV-1; at least 1-20 IU/mL of HIV-2; at least 1-50 IU/mL of HCV; at least 1-10 IU/mL of HBV; and at least 1-10 IU/mL of HAV. In certain embodiments, the plurality of pathogens or infectious agents are HIV-1, HIV-2, HCV, HBV, and HEV; and the predetermined levels are at least 1-50 copies/mL of HIV-1; at least 1-20 IU/mL of HIV-2; at least 1-50 IU/mL of HCV; at least 1-10 IU/mL of HBV; and at least 1-20 IU/mL HEV. In certain embodiments, the plurality of pathogens or infectious agents are HIV-1, HIV-2, HCV, HBV, and Zika Virus; and the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

In certain embodiments, the plurality of pathogens or infectious agents are HIV-1, HIV-2, HCV, HBV, Zika Virus, and Dengue Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus. In certain embodiments, the plurality of pathogens or infectious agents are HIV-1, HIV-2, HCV, HBV, Zika Virus, Dengue Virus, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus. In certain embodiments, the plurality of pathogens or infectious agents are HIV-1, HIV-2, HCV, HBV, Zika Virus, Dengue Virus, and WNV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV. In certain embodiments, the plurality of pathogens or infectious agents are HIV-1, HIV-2, HCV, HBV, Zika Virus, WNV, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus. In certain embodiments, the plurality of pathogens or infectious agents are HIV-1, HIV-2, HCV, HBV, Zika Virus, WNV, Dengue Virus, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

In certain embodiments, the plurality of pathogens or infectious agents are HIV-1, HIV-2, HCV, HBV, and Malaria; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria. In certain embodiments, the plurality of pathogens or infectious agents are HIV-1, HIV-2, HCV, HBV, Malaria, and Babesia; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia. In certain embodiments, the plurality of pathogens or infectious agents are HIV-1, HIV-2, HCV, HBV, and Dengue Virus; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus. In certain embodiments, the plurality of pathogens or infectious agents are HIV-1, HIV-2, HCV, HBV, Dengue Virus, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus. In certain embodiments, the plurality of pathogens or infectious agents are HIV-1, HIV-2, HCV, HBV, Dengue Virus, WNV, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus. In certain embodiments, the plurality of pathogens or infectious agents are HIV-1, HIV-2, HCV, HBV, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

In certain embodiments, the plurality of pathogens or infectious agents are HIV-1, HIV-2, HCV, HBV, Chikungunya Virus, and Zika Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus. In certain embodiments, the plurality of pathogens or infectious agents are HIV-1, HIV-2, HCV, HBV, Chikungunya Virus, Zika Virus, and WNV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV. In certain embodiments, the plurality of pathogens or infectious agents are HIV-1, HIV-2, HCV, HBV, WNV, and Dengue Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus. In certain embodiments, the plurality of pathogens or infectious agents are HIV-1, HIV-2, HCV, and HBV; the nucleic acid analysis comprises multiplex analysis of HIV-1 and HIV-2; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HIV-1, HIV-2, and HCV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HIV-1, HIV-2, and HBV; and the predetermined levels are at least 1-50 copies/mL of HIV-1; at least 1-20 IU/mL of HIV-2; at least 1-50 IU/mL of HCV; and at least 1-10 IU/mL of HBV.

In certain embodiments, the plurality of pathogens or infectious agents are HIV-1, HIV-2, HCV, and HBV; the nucleic acid analysis comprises multiplex analysis of HCV, and HBV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HIV-1, HIV-2, HCV, and HBV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents comprise Zika Virus, WNV, Chikungunya Virus and Dengue Virus; the nucleic acid analysis comprises multiplex analysis of Zika Virus and WNV; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Babesia and Malaria; the nucleic acid analysis comprises multiplex analysis of Babesia and Malaria; and the predetermined levels are at least 1-50 copies/mL of Malaria; and at least 1-20 copies/mL of Babesia. In certain embodiments, the plurality of pathogens or infectious agents comprise Parvovirus B19 and HAV; the nucleic acid analysis comprises multiplex analysis of Parvovirus B19 and HAV; and the predetermined levels are at least 1-40 IU/mL of Parvovirus B19; and at least 1-10 IU/mL of HAV.

In certain embodiments, the sample of donor blood is human donor blood. In certain embodiments, the sample of donor blood is whole blood. In certain embodiments, the sample of donor blood is lysed whole blood. In certain embodiments, the sample of donor blood is serum. In certain embodiments, the sample of donor blood is plasma.

In certain embodiments, the sample of donor blood is pooled. In certain embodiments, the pooled sample of donor blood comprises blood from 2 donors. In certain embodiments, the pooled sample of donor blood comprises blood from 3 donors. In certain embodiments, the pooled sample of donor blood comprises blood from 4 donors. In certain embodiments, the pooled sample of donor blood comprises blood from 5 donors. In certain embodiments, the pooled sample of donor blood comprises blood from 6 donors. In certain embodiments, the pooled sample of donor blood comprises blood from 8 donors. In certain embodiments, the pooled sample of donor blood comprises blood from 10 donors. In certain embodiments, the pooled sample of donor blood comprises blood from 12 donors. In certain embodiments, the pooled sample of donor blood comprises blood from 18 donors. In certain embodiments, the pooled sample of donor blood comprises blood from 24 donors. In certain embodiments, the pooled sample of donor blood comprises blood from 48 donors. In certain embodiments, the pooled sample of donor blood comprises blood from 96 donors.

In certain embodiments, the release of donor blood is for transfusion. In certain embodiments, the release of donor blood is for use in a pharmaceutical. In certain embodiments, the release of donor blood is for use in a therapeutic treatment. In certain embodiments, the sample of donor blood is for use as a blood donation. In certain embodiments, the clinical use is transfusion. In certain embodiments, the clinical use is use in a pharmaceutical. In certain embodiments, the clinical use is use in a therapeutic treatment. In certain embodiments, the clinical use is use in disease diagnostics or in quality assurance/laboratory diagnostics.

The present disclosure provides automated systems for screening one or more samples of donor blood for release of the donor blood or a material from the donor for clinical use. In certain embodiments, the automated system comprises a sample analysis station comprising, a sample loading area, a sample preparation area, a nucleic acid amplification area and a nucleic acid detection area; a processor and memory comprising instructions, when executed by the processor cause the system to perform a nucleic acid analysis on the one or more samples of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor blood or donor material for clinical use; and wherein the release of the donor blood or donor material for clinical use occurs in about 15 to about 60 minutes, e.g., about 20 to about 60 minutes, from initial aspiration of the one or more samples for performance of the nucleic acid analysis. In certain embodiments, the nucleic acid amplification area and the nucleic acid detection area can be a single area, e.g., an amplification and detection system.

The present disclosure also provides automated systems for screening one or more samples of donor blood for release of the donor blood or a material from the donor for clinical use, comprising a sample analysis station comprising, a sample loading area, a sample preparation area, a nucleic acid amplification area and a nucleic acid detection area; a processor and memory comprising instructions, when executed by the processor cause the system to perform a nucleic acid analysis on the one or more samples of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor blood or the donor material for clinical use; and wherein the system is configured to screen a plurality of samples of donor blood for release of the donor blood or donor material for clinical use and the determinations based on the nucleic acid analyses are performed within about 15 minutes to about 3.5 hours, e.g., about 20 minutes to about 3.5 hours, from initial aspiration of the first sample for performance of the nucleic acid analysis.

The present disclosure further provides automated systems for screening a one or more samples of donor blood for release of the donor blood or a material from the donor for clinical use, comprising a sample analysis station comprising, a sample loading area, a sample preparation area, a nucleic acid amplification area and a nucleic acid detection area; a processor and memory comprising instructions, when executed by the processor cause the system to perform a nucleic acid analysis on the one or more samples of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor blood or donor material for clinical use; and wherein the nucleic acid analysis comprises a nucleic acid amplification reaction of about 1 minute to about 20 minutes in duration, e.g., about 8 minutes to about 20 minutes in duration.

The present disclosure provides automated systems for screening one or more samples of donor blood for release of the donor blood or a material from the donor for clinical use, comprising a sample analysis station comprising, a sample loading area, a sample preparation area, a nucleic acid amplification area and a nucleic acid detection area; a processor and memory comprising instructions, when executed by the processor cause the system to perform a nucleic acid analysis on the one or more samples of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor blood or donor material for clinical use; and wherein each determination of a predetermined level of nucleic acid from at least one of the plurality of pathogens or infectious agent is completed in about 30 to about 45 minutes from initial aspiration of the sample for performance of the nucleic acid analysis.

The present disclosure provides automated systems for screening one or more samples of donor blood for release of the donor blood or a material from the donor for clinical use, comprising a sample analysis station comprising, a sample loading area, a sample preparation area, a nucleic acid amplification area and a nucleic acid detection area; a processor and memory comprising instructions, when executed by the processor cause the system to perform a nucleic acid analysis on the one or more samples of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor blood or donor material for clinical use; and wherein each determination of a predetermined level of nucleic acid from at least one of the plurality of pathogens or infectious agent has a time to result of about 30 to about 45 minutes.

The present disclosure provides automated systems for screening samples of donor blood for release of the donor blood or a material from the donor for clinical use, comprising a sample analysis station comprising, a sample loading area, a sample preparation area, a nucleic acid amplification area and a nucleic acid detection area; a processor and memory comprising instructions, when executed by the processor cause the system to perform a nucleic acid analysis on each of the samples of donor blood to detect one or more of a plurality of pathogens or infectious agents; wherein upon a determination of a predetermined level of nucleic acids derived from each of the plurality of pathogens or infectious agents as a result of the nucleic acid analysis, the system is indicative of release of the donor blood or donor material for clinical use; and wherein at least about 70 results are obtained per hour per m³ of a volume occupied by the automated system.

The present disclosure provides automated systems for screening samples of donor blood for release of the donor blood or a material from the donor for clinical use, comprising a sample analysis station comprising, a sample loading area, a sample preparation area, a nucleic acid amplification area and a nucleic acid detection area; a processor and memory comprising instructions, when executed by the processor cause the system to perform a nucleic acid analysis on each of the sample of donor blood to detect one or more of a plurality of pathogens or infectious agents; wherein upon a determination of a predetermined level of nucleic acids derived from each of the plurality of pathogens or infectious agents as a result of the nucleic acid analysis, the system is indicative of release of the donor blood or donor material for clinical use; and wherein at least about 140 results are obtained per hour per m² of a footprint of the automated system.

The present disclosure further provides automated systems for screening one or more sample of donor blood for release of the donor blood or a material from the donor for clinical use, comprising a sample analysis station comprising, a sample loading area, a sample preparation area, a nucleic acid amplification area and a nucleic acid detection area; a processor and memory comprising instructions, when executed by the processor cause the system to perform a nucleic acid analysis on the one or more samples of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor blood or donor material for clinical use; and wherein the system is configured to screen a plurality of samples of donor blood for release of the donor blood or donor material for clinical use and the nucleic acid analysis is performed in the absence of batching of the plurality of the samples.

In certain embodiments, the nucleic acid analysis comprises optical detection of the presence of a nucleic acid derived from at least one of the plurality of pathogens or infectious agents. In certain embodiments, the system is configured to optically detect the presence of a nucleic acid derived from at least one of the plurality of pathogens or infectious agents a plurality of times, e.g., to quantitatively detect the presence of anucleic acid derived from at least one of the plurality of pathogens or infectious agents. In certain embodiments, the nucleic acid analysis comprises a nucleic acid amplification reaction. In certain embodiments, the nucleic acid amplification reaction is an isothermal reaction. In certain embodiments, the isothermal reaction is recombinase polymerase amplification. In certain embodiments, the nucleic acid analysis comprises digital detection of the presence of a nucleic acid derived from at least one of the plurality of pathogens or infectious agents.

In certain embodiments, the system is configured to digitally detect the presence of a nucleic acid derived from at least one of the plurality of pathogens or infectious agents a plurality of times, e.g., to quantitatively detect the presence of a nucleic acid derived from at least one of the plurality of pathogens or infectious agents. In certain embodiments, the nucleic acid analysis comprises contemporaneous contact of the sample with sample lysis buffer and, optionally, a protease. In certain embodiments, the isothermal reaction is recombinase polymerase amplification. In certain embodiments, the isothermal reaction is a nicking enzyme amplification reaction.

In certain embodiments, the plurality of pathogens or infectious agents are selected from the group consisting of: SARS-CoV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, Usutu virus and HEV. In certain embodiments, the plurality of pathogens or infectious agents are HIV-1, HIV-2, HCV, and HBV. In certain embodiments, the plurality of pathogens or infectious agents are HIV-1, HIV-2, HCV, HBV, and WNV. In certain embodiments, the plurality of pathogens or infectious agents are Zika Virus and WNV.

In certain embodiments, the plurality of pathogens or infectious agents are Chikungunya Virus and Dengue Virus. In certain embodiments, the plurality of pathogens or infectious agents are Zika Virus, WNV, Chikungunya Virus and Dengue Virus. In certain embodiments, the plurality of pathogens or infectious agents are Babesia and Malaria. In certain embodiments, the plurality of pathogens or infectious agents are Parvovirus B19 and HAV.

In certain embodiments, the plurality of pathogens or infectious agents are HIV-1, HIV-2, HCV, HBV, WNV, and Zika Virus. In certain embodiments, the plurality of pathogens or infectious agents are HIV-1, HIV-2, HCV, HBV, WNV, Zika Virus, and Chikungunya Virus. In certain embodiments, the plurality of pathogens or infectious agents are HIV-1, HIV-2, HCV, HBV, WNV, Zika Virus, Chikungunya Virus, and Dengue Virus. In certain embodiments, the plurality of pathogens or infectious agents are HIV-1, HIV-2, HCV, HBV, WNV, Zika Virus, Chikungunya Virus, Dengue Virus, and Babesia. In certain embodiments, the plurality of pathogens or infectious agents are HIV-1, HIV-2, HCV, HBV, WNV, Zika Virus, Chikungunya Virus, Dengue Virus, Babesia, and Malaria. In certain embodiments, the plurality of pathogens or infectious agents are HIV-1, HIV-2, HCV, HBV, WNV, Zika Virus, Chikungunya Virus, Dengue Virus, Babesia, Malaria, and Parvovirus B19. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV. In certain embodiments, the plurality of pathogens or infectious agents are HIV-1, HIV-2, HCV, HBV, and Parvovirus B19. In certain embodiments, the plurality of pathogens or infectious agents are HIV-1, HIV-2, HCV, HBV, Parvovirus B19, and HAV.

In certain embodiments, the plurality of pathogens or infectious agents are HIV-1, HIV-2, HCV, HBV, and Babesia. In certain embodiments, the plurality of pathogens or infectious agents are HIV-1, HIV-2, HCV, HBV, and HAV. In certain embodiments, the plurality of pathogens or infectious agents are HIV-1, HIV-2, HCV, HBV, and HEV. In certain embodiments, the plurality of pathogens or infectious agents are HIV-1, HIV-2, HCV, HBV, and Zika Virus. In certain embodiments, the plurality of pathogens or infectious agents are HIV-1, HIV-2, HCV, HBV, Zika Virus, and Dengue Virus.

In certain embodiments, the plurality of pathogens or infectious agents are HIV-1, HIV-2, HCV, HBV, Zika Virus, Dengue Virus, and Chikungunya Virus. In certain embodiments, the plurality of pathogens or infectious agents are HIV-1, HIV-2, HCV, HBV, Zika Virus, Dengue Virus, and WNV. In certain embodiments, the plurality of pathogens or infectious agents are HIV-1, HIV-2, HCV, HBV, Zika Virus, WNV, and Chikungunya Virus. In certain embodiments, the plurality of pathogens or infectious agents are HIV-1, HIV-2, HCV, HBV, Zika Virus, WNV, Dengue Virus, and Chikungunya Virus.

In certain embodiments, the plurality of pathogens or infectious agents are HIV-1, HIV-2, HCV, HBV, and Malaria. In certain embodiments, the plurality of pathogens or infectious agents are HIV-1, HIV-2, HCV, HBV, Malaria, and Babesia. In certain embodiments, the plurality of pathogens or infectious agents are HIV-1, HIV-2, HCV, HBV, and Dengue Virus. In certain embodiments, the plurality of pathogens or infectious agents are HIV-1, HIV-2, HCV, HBV, Dengue Virus, and Chikungunya Virus. In certain embodiments, the plurality of pathogens or infectious agents are HIV-1, HIV-2, HCV, HBV, Dengue Virus, WNV, and Chikungunya Virus.

In certain embodiments, the plurality of pathogens or infectious agents are HIV-1, HIV-2, HCV, HBV, and Chikungunya Virus. In certain embodiments, the plurality of pathogens or infectious agents are HIV-1, HIV-2, HCV, HBV, Chikungunya Virus, and Zika Virus. In certain embodiments, the plurality of pathogens or infectious agents are HIV-1, HIV-2, HCV, HBV, Chikungunya Virus, Zika Virus, and WNV. In certain embodiments, the plurality of pathogens or infectious agents are HIV-1, HIV-2, HCV, HBV, WNV, and Dengue Virus. In certain embodiments, the plurality of pathogens or infectious agents are HIV-1, HIV-2, HCV, HBV, Chikungunya Virus, Zika Virus, and WNV.

In certain embodiments, the plurality of pathogens or infectious agents are HIV-1, HIV-2, HCV, and HBV; and the nucleic acid analysis comprises multiplex analysis of HIV-1 and HIV-2. In certain embodiments, the plurality of pathogens or infectious agents are HIV-1, HIV-2, HCV, and HBV; and the nucleic acid analysis comprises multiplex analysis of HIV-1, HIV-2, and HCV. In certain embodiments, the plurality of pathogens or infectious agents are HIV-1, HIV-2, HCV, and HBV; and the nucleic acid analysis comprises multiplex analysis of HIV-1, HIV-2, and HBV. In certain embodiments, the plurality of pathogens or infectious agents are HIV-1, HIV-2, HCV, and HBV; and the nucleic acid analysis comprises multiplex analysis of HCV, and HBV. In certain embodiments, the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

In certain embodiments, the plurality of pathogens or infectious agents are HIV-1, HIV-2, HCV, and HBV; and the nucleic acid analysis comprises multiplex analysis of HIV-1, HIV-2, HCV, and HBV. In certain embodiments, the plurality of pathogens or infectious agents comprise Zika Virus, WNV, Chikungunya Virus and Dengue Virus; and the nucleic acid analysis comprises multiplex analysis of Zika Virus and WNV. In certain embodiments, the plurality of pathogens or infectious agents comprise Zika Virus, WNV, Chikungunya Virus and Dengue Virus; and the nucleic acid analysis comprises multiplex analysis of Chikungunya Virus and Dengue Virus. In certain embodiments, the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

In certain embodiments, the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus. In certain embodiments, the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV. In certain embodiments, the plurality of pathogens or infectious agents comprise Babesia and Malaria; and the nucleic acid analysis comprises multiplex analysis of Babesia and Malaria. In certain embodiments, the plurality of pathogens or infectious agents comprise Parvovirus B19 and HAV; and the nucleic acid analysis comprises multiplex analysis of Parvovirus B19 and HAV.

In certain embodiments, the nucleic acid analysis comprises contacting the sample with CuTi-coated microparticles, e.g., during a sample preparation process. In certain embodiments, the nucleic acid analysis comprises contacting the sample with a plurality of microparticles and translation of the microparticles on a surface via magnetic force. In certain embodiments, the nucleic acid analysis comprises purification of nucleic acid from the sample of donor blood; division of the purified nucleic acid into a plurality of fractions; and at least one fraction is reserved for further screening.

In certain embodiments, the plurality of pathogens or infectious agents and predetermined levels are selected from the following SARS-CoV-2 (COVID-19) at a predetermined level of at least 1-50 copies/mL; HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; CMV at a predetermined level of at least 10-50 IU/mL; Parvovirus B19 at a predetermined level of at least 1-40 IU/mL; HAV at a predetermined level of at least 1-10 IU/mL; Chlamydia at a predetermined level of at least 100-500 copies/mL; Gonorrhea at a predetermined level of at least 100-500 copies/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Influenza at a predetermined level of at least 10-500 copies/mL; Babesia at a predetermined level of at least 1-20 copies/mL; Malaria at a predetermined level of at least 1-50 copies/mL; and HEV at a predetermined level of at least 1-20 IU/mL.

In certain embodiments, the plurality of pathogens or infectious agents are HIV-1, HIV-2, HCV, and HBV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, HBV, and WNV; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of WNV. In certain embodiments, the plurality of pathogens or infectious agents are Zika Virus and WNV; and the predetermined levels are at least 1-50 copies/mL of Zika Virus; and at least 1-50 copies/mL of WNV. In certain embodiments, the plurality of pathogens or infectious agents are Chikungunya Virus and Dengue Virus; and the predetermined levels are at least 1-50 copies/mL of Chikungunya Virus; and at least 1-50 copies/mL of Dengue Virus. In certain embodiments, the plurality of pathogens or infectious agents are Zika Virus, WNV, Chikungunya Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the plurality of pathogens or infectious agents are Babesia and Malaria; and wherein the predetermined levels are at least 1-20 copies/mL of Babesia; and at least 1-50 copies/mL of Malaria. In certain embodiments, the plurality of pathogens or infectious agents are Parvovirus B19 and HAV; and the predetermined levels are at least 1-40 IU/mL of Parvovirus B19; and at least 1-10 IU/mL of HAV. In certain embodiments, the plurality of pathogens or infectious agents are HIV-1, HIV-2, HCV, HBV, WNV, and Zika Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus. In certain embodiments, the plurality of pathogens or infectious agents are HIV-1, HIV-2, HCV, HBV, WNV, Zika Virus, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus. In certain embodiments, the plurality of pathogens or infectious agents are HIV-1, HIV-2, HCV, HBV, WNV, Zika Virus, Chikungunya Virus, and Dengue Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus. In certain embodiments, the plurality of pathogens or infectious agents are HIV-1, HIV-2, HCV, HBV, WNV, Zika Virus, Chikungunya Virus, Dengue Virus, and Babesia; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

In certain embodiments, the plurality of pathogens or infectious agents are HIV-1, HIV-2, HCV, HBV, WNV, Zika Virus, Chikungunya Virus, Dengue Virus, Babesia, and Malaria; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria. In certain embodiments, the plurality of pathogens or infectious agents are HIV-1, HIV-2, HCV, HBV, WNV, Zika Virus, Chikungunya Virus, Dengue Virus, Babesia, Malaria, and Parvovirus B19; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; at least 1-50 copies/mL of Malaria; and at least 1-40 IU/mL of Parvovirus B19. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; at least 1-50 copies/mL of Malaria; at least 1-40 IU/mL of Parvovirus B19; and at least 1-10 IU/mL of HAV. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV. In certain embodiments, the plurality of pathogens or infectious agents are HIV-1, HIV-2, HCV, HBV, and Parvovirus B19; and the predetermined levels are at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

In certain embodiments, the plurality of pathogens or infectious agents are HIV-1, HIV-2, HCV, HBV, Parvovirus B19, and HAV; and the predetermined levels are at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV. In certain embodiments, the plurality of pathogens or infectious agents are HIV-1, HIV-2, HCV, HBV, and Babesia; and the predetermined levels are at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia. In certain embodiments, the plurality of pathogens or infectious agents are HIV-1, HIV-2, HCV, HBV, and HAV; and the predetermined levels are at least 1-50 copies/mL of HIV-1; at least 1-20 IU/mL of HIV-2; at least 1-50 IU/mL of HCV; at least 1-10 IU/mL of HBV; and at least 1-10 IU/mL of HAV. In certain embodiments, the plurality of pathogens or infectious agents are HIV-1, HIV-2, HCV, HBV, and HEV; and the predetermined levels are at least 1-50 copies/mL of HIV-1; at least 1-20 IU/mL of HIV-2; at least 1-50 IU/mL of HCV; at least 1-10 IU/mL of HBV; and at least 1-20 IU/mL HEV. In certain embodiments, the plurality of pathogens or infectious agents are HIV-1, HIV-2, HCV, HBV, and Zika Virus; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus. In certain embodiments, the plurality of pathogens or infectious agents are HIV-1, HIV-2, HCV, HBV, Zika Virus, and Dengue Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

In certain embodiments, the plurality of pathogens or infectious agents are HIV-1, HIV-2, HCV, HBV, Zika Virus, Dengue Virus, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus. In certain embodiments, the plurality of pathogens or infectious agents are HIV-1, HIV-2, HCV, HBV, Zika Virus, Dengue Virus, and WNV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV. In certain embodiments, the plurality of pathogens or infectious agents are HIV-1, HIV-2, HCV, HBV, Zika Virus, WNV, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus. In certain embodiments, the plurality of pathogens or infectious agents are HIV-1, HIV-2, HCV, HBV, Zika Virus, WNV, Dengue Virus, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus. In certain embodiments, the plurality of pathogens or infectious agents are HIV-1, HIV-2, HCV, HBV, and Malaria; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria. In certain embodiments, the plurality of pathogens or infectious agents are HIV-1, HIV-2, HCV, HBV, Malaria, and Babesia; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

In certain embodiments, the plurality of pathogens or infectious agents are HIV-1, HIV-2, HCV, HBV, and Dengue Virus; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus. In certain embodiments, the plurality of pathogens or infectious agents are HIV-1, HIV-2, HCV, HBV, Dengue Virus, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus. In certain embodiments, the plurality of pathogens or infectious agents are HIV-1, HIV-2, HCV, HBV, Dengue Virus, WNV, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus. In certain embodiments, the plurality of pathogens or infectious agents are HIV-1, HIV-2, HCV, HBV, and Chikungunya Virus; and the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus. In certain embodiments, the plurality of pathogens or infectious agents are HIV-1, HIV-2, HCV, HBV, Chikungunya Virus, and Zika Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

In certain embodiments, the plurality of pathogens or infectious agents are HIV-1, HIV-2, HCV, HBV, Chikungunya Virus, Zika Virus, and WNV; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV. In certain embodiments, the plurality of pathogens or infectious agents are HIV-1, HIV-2, HCV, HBV, WNV, and Dengue Virus; and the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus. In certain embodiments, the plurality of pathogens or infectious agents are HIV-1, HIV-2, HCV, and HBV; the nucleic acid analysis comprises multiplex analysis of HIV-1 and HIV-2; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HIV-1, HIV-2, and HCV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HIV-1, HIV-2, and HBV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HCV, and HBV; and the predetermined levels are at least 1-50 copies/mL of HIV-1; at least 1-20 IU/mL of HIV-2; at least 1-50 IU/mL of HCV; and at least 1-10 IU/mL of HBV.

In certain embodiments, the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents are HIV-1, HIV-2, HCV, and HBV; the nucleic acid analysis comprises multiplex analysis of HIV-1, HIV-2, HCV, and HBV; and the predetermined levels are at least 1-50 copies/mL of HIV-1; at least 1-20 IU/mL of HIV-2; at least 1-50 IU/mL of HCV; and at least 1-10 IU/mL of HBV. In certain embodiments, the plurality of pathogens or infectious agents comprise Zika Virus, WNV, Chikungunya Virus and Dengue Virus; the nucleic acid analysis comprises multiplex analysis of Zika Virus and WNV; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus. In certain embodiments, the plurality of pathogens or infectious agents comprise Babesia and Malaria; the nucleic acid analysis comprises multiplex analysis of Babesia and Malaria; and the predetermined levels are at least 1-50 copies/mL of Malaria; and at least 1-20 copies/mL of Babesia. In certain embodiments, the plurality of pathogens or infectious agents comprise Parvovirus B19 and HAV; the nucleic acid analysis comprises multiplex analysis of Parvovirus B19 and HAV; and the predetermined levels are at least 1-40 IU/mL of Parvovirus B19; and at least 1-10 IU/mL of HAV.

In certain embodiments, the sample of donor blood is human donor blood. In certain embodiments, the sample of donor blood is whole blood. In certain embodiments, the sample of donor blood is lysed whole blood. In certain embodiments, the sample of donor blood is serum. In certain embodiments, the sample of donor blood is plasma.

In certain embodiments, the sample of donor blood is pooled. In certain embodiments, the pooled sample of donor blood comprises blood from 2 donors. In certain embodiments, the pooled sample of donor blood comprises blood from 3 donors. In certain embodiments, the pooled sample of donor blood comprises blood from 4 donors. In certain embodiments, the pooled sample of donor blood comprises blood from 5 donors. In certain embodiments, the pooled sample of donor blood comprises blood from 6 donors. In certain embodiments, the pooled sample of donor blood comprises blood from 8 donors. In certain embodiments, the pooled sample of donor blood comprises blood from 10 donors. In certain embodiments, the pooled sample of donor blood comprises blood from 18 donors. In certain embodiments, the pooled sample of donor blood comprises blood from 12 donors. In certain embodiments, the pooled sample of donor blood comprises blood from 24 donors. In certain embodiments, the pooled sample of donor blood comprises blood from 48 donors. In certain embodiments, the pooled sample of donor blood comprises blood from 96 donors.

In certain embodiments, the release of donor blood is for transfusion. In certain embodiments, the release of donor blood is for use in a pharmaceutical. In certain embodiments, the release of donor blood is for use in a therapeutic treatment. In certain embodiments, the sample of donor blood is for use as a blood donation. In certain embodiments, the clinical use is transfusion. In certain embodiments, the clinical use is use in a pharmaceutical. In certain embodiments, the clinical use is use in a therapeutic treatment.

The present disclosure provides methods of washing microparticles for nucleic acid analysis, comprising providing a wash vessel comprising a first well, a second well adjacent the first well, and a third well adjacent the second well, wherein a first side wall defines a first side of the first, second, and third wells, a second side wall opposite the first side wall, defines a second side of the first, second, and third wells, the first and second side walls comprise an inner surface facing the first, second, and third wells and an outer surface opposite the inner surface, a first divider separates the first and second well, the first side wall is higher than the first divider, the inner surface of the first side wall is substantially planar at least in a region extending between the first and second wells, a second divider separates the second and third wells, the inner surface of the second side wall is substantially planar at least in a region extending between the second and third wells, introducing the magnetic microparticles into the first well, wherein the first well comprises a wash solution, applying a magnetic force to capture the magnetic microparticles on an inner surface of the first side wall in the first well; translating the captured magnetic microparticles from the first well over the first divider to the second well along the inner planar surface of the first side wall, wherein the second well comprises a wash solution; removing the magnetic force and allowing the magnetic microparticles to be released into the second well from the inner surface; applying a magnetic force to capture the magnetic microparticles on the inner surface of the second side wall opposite the first side wall, wherein the second side wall is higher than the second divider, translating the captured magnetic microparticles from the second well over the second divider to the third well along the inner planar surface of the second side wall, wherein the third well comprises a wash solution or an elution solution; and removing the magnetic force and allowing the magnetic microparticles to be released into the third well from the inner surface of the second side wall. In certain embodiments, the method of washing microparticles disclosed herein can be used during a sample preparation process.

In certain embodiments, the third well comprises an elution solution and the method further comprises, applying a magnetic force to the magnetic microparticles in the third well to capture the magnetic microparticles on an inner surface of the first side wall and removing the elution solution. In certain embodiments, the wash vessel comprises a fourth well and wherein the third well comprises a wash solution and the fourth well comprises an elution solution, and the third and fourth wells are separated by a third divider. In certain embodiments, the first and second side walls extend along the first, second, third and fourth wells and define opposite sides of the wells, the inner surface of the first and second side wall faces the wells, the inner surface of the first side wall is non-planar in a region extending between the second and third wells, and planar in a region extending between the third and the fourth wells, the inner surface of the second side wall is non-planar in a region extending between the third and fourth wells, and the planar regions are conducive to translation of captured magnetic microparticles across the inner surface and the non-planar regions do not allow translation of captured magnetic microparticles across the inner surface.

In certain embodiments, the methods further comprise applying a magnetic force to capture the magnetic particles in the third well on an inner surface of the first side wall, wherein the first side wall is higher than the third divider; translating the captured magnetic microparticles from the third well over the third divider to the fourth well along the inner surface of the first side wall; and removing the magnetic force and allowing the magnetic microparticles to be released into the fourth well from the inner surface of the first side wall. In certain embodiments, the third well comprises a wash solution and the fourth well comprises an elution solution and the method further comprises applying a magnetic force to the magnetic microparticles in the fourth well to capture the magnetic microparticles on an inner surface of the second side wall and removing the elution solution. In certain embodiments, the magnetic force is produced using an electromagnet. In certain embodiments, the electromagnet is located along the outer surface of the first and second side walls and the translating the captured magnetic microparticles comprises sequentially activating and deactivating different regions of the electromagnet. In certain embodiments, the magnetic force is produced using a permanent magnet. In certain embodiments, the translating the captured magnetic microparticles comprises moving the permanent magnet along the outer surface of the first or the second side wall. In certain embodiments, the non-planar inner surface comprises a notch at the inner surface, wherein the notch extends out of the inner surface or extends into the inner surface.

The present disclosure also provides a wash vessel comprising a first well adjacent a second well and a third well adjacent the second well, a first side wall defining a first side of the first, second, and third wells, a second side wall opposite the first side wall and defining a second side of the first, second, and third wells, the first and second side walls comprising an inner surface facing the first, second, and third wells and an outer surface opposite the inner surface, a first divider separating the first and second well, a second divider separating the second and third wells, wherein the first side wall is higher than the first divider, the inner surface of the first side wall is substantially planar at least in a region extending between the first and second wells for translation of magnetic microparticles captured in the first well over the first divider into the second well, the inner surface of the first side wall is non-planar in a region extending between the second and third wells such that the non-planar inner surface prevents translation of magnetic particles captured at a region of the inner surface in the second well to a region of the inner surface in the third well, the second side wall is higher than the second divider, the inner surface of the second side wall is substantially planar at least in a region extending between the second and third wells and provides a substantially planar surface for capture of magnetic microparticles in the second well and translation of the captured magnetic particles over the second divider to the third well.

In certain embodiments, the first and second wells comprise a wash solution and the third well comprises a wash solution or an elution solution. In certain embodiments, the wash vessel comprises a fourth well adjacent the third well, a third divider separating the third and fourth wells, wherein the first side wall defines a first side of the fourth well and the second side wall defines a second side the fourth well, the first side wall is higher than the third divider, the inner surface of the first side wall is substantially planar at least in a region extending between the third and fourth wells for translation of magnetic microparticles captured in the third well over the third divider into the fourth well, and the inner surface of the second side wall is non-planar in a region extending between the third and fourth wells such that the non-planar inner surface prevents translation of magnetic particles captured at a region of the inner surface of the second side wall in the third well to a region of the inner surface of the second side wall in the fourth well. In certain embodiments, the first, second, and third wells comprise wash solution. In certain embodiments, the first, second, and third wells comprise wash solution and the fourth well comprises an elution solution. In certain embodiments, the non-planar inner surface comprises a notch at the inner surface, wherein the notch extends out of the inner surface or extends into the inner surface.

The present disclosure further provides methods of screening a sample associated with donor blood for release of the donor blood or a material from the donor for clinical use, comprising preparing the sample for nucleic acid analysis comprising providing nucleic acid isolated from the sample in a volume of eluate; and dispensing the volume of eluate into at least two amplification vessels. In certain embodiments, the method can further include subjecting the eluate in the at least two amplification vessels to a nucleic acid amplification reaction (e.g., during an amplification and detection process); and detecting whether nucleic acid of a pathogen or an infectious agent is present or absent in the eluate.

In certain embodiments, the dispensing comprises dispensing the volume of eluate into at least three, four, or more amplification vessels; subjecting the eluate in the amplification vessels to a nucleic acid amplification reaction (e.g., during an amplification and detection process); and detecting whether nucleic acid of a pathogen or an infectious agent is present or absent in the eluate. In certain embodiments, an equal volume of eluate is dispensed into the amplification vessels. In certain embodiments, different volumes of the eluate are dispensed into the amplification vessels. In certain embodiments, the amplification reaction in each amplification vessel is for amplification of nucleic acid present in a different pathogen or infectious agent. In certain embodiments, the amplification reaction in each amplification vessel is for amplification of nucleic acid present in a plurality of different pathogens or infectious agents. In certain embodiments, the amplification reaction comprises an isothermal reaction. In certain embodiments, the isothermal reaction is recombinase polymerase amplification. In certain embodiments, the isothermal reaction is a nicking enzyme amplification reaction.

The present disclosure further provides methods for the detection of a target nucleic acid in a sample. For example, but not by way of limitation, the method can include preparing nucleic acid from the sample for isothermal amplification reaction; amplifying the target nucleic acid with an isothermal amplification reaction; and determining an amount of the target nucleic acid amplified in the reaction and/or determining the presence or absence of the target nucleic acid amplified in the reaction, wherein the sample preparation, amplification reaction and determining the amount, presence or absence of the target nucleic acid amplified in the amplification reaction are completed in less than about 60 minutes, e.g., completed in less than about 20 minutes. In certain embodiments, the present disclosure provides methods for the detection of a target nucleic acid of a pathogen or infectious agent in a sample, comprising preparing nucleic acid from the sample for isothermal amplification reaction; amplifying the target nucleic acid with an isothermal amplification reaction; and determining an amount of the target nucleic acid amplified in the reaction and/or determining the presence or absence of the target nucleic acid amplified in the reaction, wherein the sample preparation, amplification reaction and determining the amount, presence or absence of the target nucleic acid amplified in the amplification reaction are completed in less than about 60 minutes, e.g., completed in less than about 20 minutes.

In certain embodiments, determining an amount of the target nucleic acid comprises optically detecting a fluorescent signal corresponding to the amount of the target nucleic acid at a plurality of times during the amplification reaction. In certain embodiments, determining the presence or absence of the target nucleic acid comprises optically detecting a fluorescent signal corresponding to the target nucleic acid at a plurality of times during the amplification reaction. In certain embodiments, the plurality of times comprises optically detecting the signal at a predetermined interval of about every 20 seconds or about every 30 seconds. In certain embodiments, the optically detecting a fluorescent signal for a plurality of times occurs over a period of about 12 minutes. In certain embodiments, the method is for screening a sample associated with donor blood for release of the donor blood for clinical use. In certain embodiments, the sample preparation, amplification reaction and determining the amount, presence or absence of the target nucleic acid amplified in the amplification reaction are completed in about 15 minutes to about 60 minutes, e.g., in about 20 minutes to about 60 minutes. In certain embodiments, the target nucleic acid comprises nucleic acid from Parvovirus B19 and hepatitis A virus (HAV). In certain embodiments, the target nucleic acid comprises nucleic acid from a plurality of pathogens or infectious agents. In certain embodiments, the plurality of pathogens or infectious agents comprise HIV-1, HIV-2, HBV, and HCV. In certain embodiments, the plurality of pathogens or infectious agents comprise HIV-1 and HBV. In certain embodiments, the plurality of pathogens or infectious agents comprise HIV-2 and HCV.

The present disclosure further provides methods of screening samples of donor blood for release of a donor material for clinical use. In certain embodiments, the donor material can be a blood product, such as for example, whole blood, platelets, red blood cells, and plasma. In certain embodiments, the donor material can be, for example, tissues, organs, vaccines, cells, gene therapy, and recombinant therapeutic proteins.

In accordance with another aspect of the disclosed subject matter, biological samples other than donor blood can be analyzed using the methods and systems of the present disclosure. For example, in certain embodiments, the biological sample is a bodily secretion, such as for example, saliva or oral fluid, sweat, tears, mucus, urine, lymphatic fluid, cerebrospinal fluid, interstitial fluid, bronchoalveolar lavage fluid or any other sample suitable for analysis using the methods and techniques described herein.

### BRIEF DESCRIPTION OF THE DRAWINGS

FIG. 1 illustrates an exemplary HTNAT sample analysis process according to certain embodiments of the disclosed subject matter and exemplary benefits associated with certain embodiments of the sample preparation, amplification, and detection strategies of the present disclosure. FIG. 1 illustrates an exemplary HTNAT sample analysis process according to certain embodiments of the disclosed subject matter comprising three processes: a sample preparation process, an amplification process, and a detection process. In certain embodiments, the amplification process and the detection process are performed simultaneously in an amplification and detection process.

FIGS. 2A-2B are diagrams illustrating an exemplary HTNAT automated sample analysis process according to the disclosed subject matter. FIG. 2A illustrates an exemplary HTNAT automated sample analysis process comprising three processes: a sample preparation process (e.g., a pre-lysis process, a sample lysis process, and a wash & elute process), an amplification process (including reagent addition), and a detection/reading process. FIG. 2B is a diagram illustrating an exemplary HTNAT automated sample preparation protocol for processing plasma/serum or whole blood sample types according to the disclosed subject matter.

FIG. 3 is a diagram illustrating an exemplary HTNAT sample analysis system according to the disclosed subject matter comprising three processes: a sample preparation process (e.g., a pre-treatment lysis process, a sample lysis process, and a wash & elute process), an amplification process, and a detection/reading process.

FIG. 4 is a diagram illustrating exemplary embodiments of the sample preparation process (direct capture and CuTi total nucleic acid capture) in accordance with the disclosed subject matter.

FIG. 5 is a diagram illustrating an exemplary pre-treatment lysis process (also referred to herein Pre-Lysis) (sample preparation) system according to the disclosed subject matter.

FIG. 6 is a diagram illustrating an exemplary Sample Lysis process (sample preparation) system according to the disclosed subject matter.

FIG. 7 is a diagram illustrating an exemplary wash process (also referred to herein as Wash & Elute) (sample preparation) system according to the disclosed subject matter.

FIG. 8 is a diagram illustrating an exemplary Amplification and Detection system according to the disclosed subject matter.

FIG. 9 is a diagram illustrating exemplary CuTi total nucleic acid capture process improvements (highlighted) of the disclosed subject matter.

FIG. 10 is a diagram illustrating exemplary direct capture process improvements of the disclosed subject matter.

FIG. 11 illustrates exemplary results associated with RPA amplification of HBV followed by Digital detection (left) or fluorescence (right) implementations of the disclosed subject matter.

FIG. 12 illustrates exemplary results associated with NEAR HCV amplification and fluorescent detection implementations of the disclosed subject matter. The table indicates oligonucleotides used.

FIG. 13 is a diagram illustrating exemplary digital and optical (fluorescence-based) target detection strategies.

FIG. 14 illustrates exemplary results associated with RPA implementations of the disclosed subject matter. The purple traces represent separate replicates within the same experiment; the grey traces are negative controls. The table indicates RPA oligonucleotides used.

FIG. 15 is a diagram of an exemplary mixing and washing embodiment, identifying eighteen (18) distinct positions at which stationary electromagnet-based capture and wash of magnetic particles can be incorporated.

FIG. 16 is a diagram of an exemplary mixing and washing embodiment, identifying four (4) pairs of positions at which stationary electromagnet-based transfer of magnetic particles can be incorporated.

FIGS. 17A-17B illustrate an exemplary design of a wash vessel for use in conjunction with a moving permanent magnet or stationary electromagnet, and the approximate volumes of the 4 wells of the vessel.

FIGS. 18A-18F are graphs of the RPA of two genes (ORFs): NS1 and VP1 of Parvovirus B19 (FIGS. 18A-18D, respectively; Cycle indicates fluorescent reads every 60 seconds) and the 5′ untranslated region (UTR) of the single HAV polypeptide (FIGS. 18E-18F) using various combinations of amplification and oligonucleotides. Each trace on the plot indicates a different replicate.

FIGS. 19A-19D are graphs of the RPA of various Babesia parasite species using various combinations of amplification and probe oligonucleotides, as indicated. Each cycle represents a fluorescent read every 60 seconds.

FIGS. 20A-20D are graphs of the RPA of human immunodeficiency virus 1 (HIV-1), human immunodeficiency virus 2 (HIV-2), hepatitis C virus (HCV), and hepatitis B virus (HBV), respectively. Each trace on the plot indicates a different replicate at the indicated concentration.

FIG. 21 is a diagram illustrating an exemplary RPA amplification protocol according to the disclosed subject matter.

FIG. 22 illustrates exemplary results associated with improved RNA detection facilitated by use of selected reverse transcriptase enzymes. HCV RNA amplification is used as an example. The boxed values indicate the target (HCV RNA) concentration and Reverse Transcriptase used.

FIGS. 23A-23B illustrates exemplary results associated with improved RPA amplification of HCV in the presence of primers directed to HBV. FIG. 23A shows the inhibition of HCV in the presence of HBV oligonucleotides; FIG. 23B shows the recovery of HCV amplification with reducing the overall concentration of HBV oligonucleotides. Not shown, HBV amplification is not affected by this change.

FIG. 24 is a graph of the singleplex RPA of HIV-1 using the amplification and probe oligonucleotides, as indicated, at levels at or near the desired Limit of Detection (LOD). Each curve represents a single replicate (n=6) at the noted target concentration.

FIG. 25 is a graph of the singleplex RPA of HIV-2 using the amplification and probe oligonucleotides, as indicated, at levels at or near the desired Limit of Detection (LOD). Each curve represents a single replicate (n=6) at the noted target concentration.

FIG. 26 is a graph of the singleplex RPA of HBV using the amplification and probe oligonucleotides, as indicated, at levels at or near the desired Limit of Detection (LOD). Each curve represents a single replicate (n=6) at the noted target concentration.

FIG. 27 is a graph of the singleplex RPA of HCV using the amplification and probe oligonucleotides, as indicated, at levels at or near the desired Limit of Detection (LOD). Each curve represents a single replicate (n=6) at 10 IU per reaction.

FIG. 28 is a graph of the multiplex RPA of HIV-1, HBV, and internal control at the indicated target levels. Of note, lx LOD for HIV-1 is 20 copies/mL, for HBV is 5 IU/mL. The numbers at the top left of the plot represent the number of positive replicates by the total number tested.

FIG. 29 is a graph of the multiplex RPA of HIV-2, HCV, and internal control at the indicated target levels. Of note, lx LOD for HIV-2 is 20 IU/mL, for HCV it is 10 IU/mL. The numbers at the top left of the plot represent the number of positive replicates by the total number tested.

FIGS. 30A-30B are graphs of the singleplex RPA of HAV (FIG. 30A) and Parvo (FIG. 30B) using the amplification and probe oligonucleotides, as indicated. Each cycle represents a fluorescent read every 30 seconds. The target level and probe fluorophore are indicated above the amplification plots.

FIGS. 31A-31D are graphs of the singleplex RPA of Parvo at distinct target nucleic acid concentrations (FIG. 31A). These data are then graphed in FIG. 31B to establish a standard curve with the cycle threshold (Ct) on the y-axis and the target level concentration in log on the x-axis (each data point representing a single replicate). FIG. 31C graphs the correlation between the calculated viral titer on the y-axis and the actual viral titer on the x-axis, as determined by the regression formula in FIG. 31B. The table (FIG. 31D) is a numerical output of the data from the plots, demonstrating acceptable linearity across the viral titers tested.

FIGS. 32A-32D depict the location of the primers and probe relative the Chikungunya Virus genome (FIG. 32A); graphs of the singleplex RPA of Chikungunya Virus using the indicated amplification primer and probe oligonucleotides as well as the target levels in copies per reaction of in vitro transcribed RNA (FIG. 32B and FIG. 32C); the sequences of the indicated primers and probe are listed in the table (FIG. 32D).

FIGS. 33A-33C include a graph of the singleplex RPA of Dengue Virus (FIG. 33A) using the indicated conditions (FIG. 33B); as well as the sequences of the indicated primers and probe (FIG. 33C).

FIGS. 34A-34E depict the location of the primers and probe relative the West Nile Virus (WNV) genome (FIG. 34A); graphs of the singleplex RPA of WNV using the indicated amplification primer and probe oligonucleotides (FIG. 34B and FIG. 34C); as well as the sequences of the indicated primers and probe (FIG. 34D); and a graph of the singleplex RPA of WNV using the indicated amplification and probe oligonucleotides at different concentrations of target nucleic acid (FIG. 34E).

FIGS. 35A-35C depict the location of the primers and probe relative the Zika Virus genome (FIG. 35A); graphs of the singleplex RPA of Zika Virus using the indicated amplification primer and probe oligonucleotides (FIG. 35B); as well as the sequences of the indicated primers and probe (FIG. 35C).

FIGS. 36A-36C include graphs of the multiplex RPA of Babesia (FIG. 36A) and an internal control (FIG. 36B) using the indicated Babesia amplification primer and probe oligonucleotides (FIG. 36C) with sequences provided in the table.

FIGS. 37A-37C include graphs of the multiplex RPA of Malaria (FIG. 37A) and an internal control (FIG. 37B) using the indicated Malaria amplification primer and probe oligonucleotides (FIG. 37C) with sequences provided in the table.

FIGS. 38A-38B include graphs of RPA amplification and detection of SARS-CoV-2 (COVID-19) targeting either the RdRp (FIG. 38A) or the N (FIG. 38B) genomic regions.

FIGS. 39A-39B include graphs of the multiplex RPA of HIV-1 (FIG. 39A) and HBV (FIG. 39B) indicating that the indicated limits of detection can be achieved using the methods of the present disclosure in the time frames indicated.

FIGS. 40A-40B include graphs of the multiplex RPA of HIV-2 (FIG. 40A) and HCV (FIG. 40B) indicating that the indicated limits of detection can be achieved using the methods of the present disclosure in the time frames indicated.

FIGS. 41A-41B include graphs of the multiplex RPA of Parvovirus B19 (FIG. 41A) and HAV (FIG. 41B) indicating that the indicated limits of detection can be achieved using the methods of the present disclosure in the time frames indicated.

FIGS. 42A-42D depict an exemplary embodiment of electromagnet-based or moving permanent magnet-based sample processing and associated strategies useful in connection with systems of the present disclosure.

FIG. 43 depicts exemplary embodiments of split eluate sample processing systems and associated strategies useful in connection with systems of the present disclosure.

FIGS. 44A-44D include graphs of the multiplex HxV RPA results for HIV-1 (FIG. 44A) and HIV-2 (FIG. 44B), HBV (FIG. 44C), and HCV (FIG. 44D) indicating that the indicated limits of detection can be achieved using the methods of the present disclosure.

FIG. 45 depicts exemplary embodiments of sample processing cartridges and associated strategies useful in connection with systems of the present disclosure.

FIGS. 46A-46C depict exemplary embodiments of heating blocks and associated heating strategies useful in connection with systems of the present disclosure.

FIGS. 47A-47B depict exemplary embodiments of overall system architecture, e.g., sample handling positions, location of reagents, and associated computer processing units, as well as associated sample processing strategies useful in connection with systems of the present disclosure.

FIG. 48 depicts exemplary embodiments of sample tubes and sample tube processing strategies, e.g., use of particular lanes, useful in connection with systems of the present disclosure.

FIG. 49 depicts exemplary embodiments of loading shelf strategies useful in connection with systems of the present disclosure.

FIG. 50 depicts exemplary embodiments of code readers strategies useful in connection with systems of the present disclosure FIG. 51 depicts exemplary embodiments of conveyer strategies useful in connection with systems of the present disclosure.

FIGS. 52A-52B depict exemplary embodiments of systems of the present disclosure relating to pipette tip rack loaders.

FIG. 53 depicts exemplary embodiments of systems of the present disclosure relating to sample preparation cartridges loading areas and related reagent vessel organization.

FIGS. 54A-54E depict exemplary sample preparation cartridge transport and sample preparation cartridge filling stations according to one embodiment.

FIGS. 55A-55C depict exemplary embodiments of systems of the present disclosure relating to robotic handling of sample preparation cartridges.

FIG. 56 depicts exemplary embodiments of systems of the present disclosure relating to the filling of sample preparation cartridges.

FIG. 57 depicts exemplary embodiments of systems of the present disclosure relating to the storage of auxiliary reagents.

FIG. 58 depicts exemplary embodiments of systems of the present disclosure relating to the storage of bulk reagents.

FIG. 59 depicts exemplary embodiments of systems of the present disclosure employing a magnet-based system, e.g., a Magtration® system, for the isolation of magnetic particles within pipette tips.

FIGS. 60A-60C depict exemplary embodiments of systems of the present disclosure wherein the amplification vessels traverse locations via tracks.

FIG. 61 depicts exemplary embodiments of systems of the present disclosure wherein the reader, e.g., an optical detector, is movable while the amplification vessel is stationary.

FIG. 62 depicts a plan view of an exemplary HTNAT sample analysis system for performing sample preparation, amplification and detection, as well as additional components for related to obtaining a result from a HTNAT sample analysis system, such as sample handling, consumable loading and waste disposal.

FIG. 63 depicts is a perspective view of an exemplary HTNAT sample analysis system for performing sample preparation, amplification and detection, as well as additional components for related to obtaining a result from a HTNAT sample analysis system, such as sample handling, consumable loading and waste disposal.

FIG. 64 depicts an exemplary sample transport for performing sample preparation in connection with obtaining a result from an exemplary HTNAT sample analysis system.

FIG. 64A depicts an exemplary schematic of a sample transport and exemplary rotational movement of the sample transport for performing sample mixing.

FIG. 64B depicts an exemplary schematic of a lysis tube during rotational movement of the sample transport depicted in FIG. 64A.

FIG. 64C depicts an exemplary schematic of a lysis tube during rotational movement of the sample transport depicted in FIG. 64A.

FIG. 65 depicts an exemplary wash and elution system for performing sample preparation in connection with obtaining a result from an exemplary HTNAT sample analysis system.

FIG. 66 depicts an exemplary amplification and detection system in connection with obtaining a result from an exemplary HTNAT sample analysis system.

FIGS. 67A-67B depict exemplary embodiments of the split eluate aspect of the present disclosure. FIG. 67A depicts the scenarios of not splitting (top process path), splitting with an odd number of assays (middle process path), and splitting with an even number of assays (bottom process path). In FIG. 67B, various scenarios of assays combinations and eluate splitting illustrate the benefit and utility of eluate splitting FIGS. 68A-68D depict an exemplary processing deck with pipettors of an exemplary HTNAT sample analysis system.

FIG. 69A depicts exemplary embodiments of lysis tubes and transfer tips and exemplary stacking configurations for lysis tubes and transfer tips for use with the HTNAT sample analysis system.

FIG. 69B depicts additional exemplary embodiments of a lysis tube and transfer tip and an exemplary stacking configuration for lysis tubes and transfer tips for use with the HTNAT sample analysis system.

FIG. 69C depicts a top view of another exemplary embodiment of a lysis tube for use with the HTNAT sample analysis system.

FIG. 69D depicts a side cross-sectional view of the exemplary lysis tube depicted in FIG. 69C, taken along line A-A as shown in FIG. 69C.

FIG. 69E depicts a top view of another exemplary embodiment of a lysis tube for use with the HTNAT sample analysis system.

FIG. 69F depicts a side cross-sectional view of the exemplary lysis tube depicted in FIG. 69E, taken along line B-B as shown in FIG. 69E.

FIG. 70 depicts an exemplary wash track of the exemplary HTNAT sample analysis system in FIG. 68A.

FIG. 71 depicts an exemplary wash vessel for use in the exemplary wash track of FIG. 43.

FIG. 72 depicts an exemplary amplification and detection system of the exemplary HTNAT sample analysis system in FIG. 68A.

FIG. 73 depicts an exemplary amplification vessel for use in the amplification and detection system of FIG. 72.

FIG. 74 depicts an exemplary sample load bay for use with the exemplary HTNAT sample analysis system.

FIG. 75 illustrates an exemplary embodiment of the load bay with sample tray racks.

FIG. 76 depicts mixing of the contents of a lysis tube using rotational movement of a rotatable carousel in accordance with an aspect of the disclosed subject matter.

FIG. 77 depicts an exemplary whole blood sample processing method using the systems and devices in accordance with the disclosed subject matter.

FIG. 78 depicts an exemplary system in accordance with the disclosed subject matter.

FIG. 79 depicts an exemplary method in accordance with the disclosed subject matter.

FIG. 80 depicts an exemplary embodiment of a wash vessel.

FIG. 81 is a top view of an exemplary sample preparation carousel of the exemplary HTNAT sample analysis system of FIGS. 68A-68D.

FIGS. 82A-82B are schematic top views of the sample preparation carousel of FIG. 81 illustrating an example of pooling of a first sample and a second sample in a tube on the sample preparation carousel in accordance with an aspect of the disclosed subject matter.

FIGS. 83A-83B are schematic top views of the sample preparation carousel of FIG. 81 illustrating an example of pooling of a third sample and a fourth sample.

FIGS. 84A-84C are schematic top views of the sample preparation carousel of FIG. 81 illustrating an exemplary pre-treatment process and pooling of pre-treated samples in accordance with another aspect of the disclosed subject matter.

FIGS. 85A-85F illustrate exemplary pool deconstruction strategies.

FIGS. 86A-86B depict exemplary throughput for exemplary sample types and processes for exemplary systems in accordance with the disclosed subject matter.

FIGS. 87A and 87B illustrate an exemplary method of onboard pooling of 12 samples in accordance with an aspect of the disclosed subject matter.

FIGS. 88A and 88B illustrate an exemplary method of onboard pooling of 18 samples in accordance with an aspect of the disclosed subject matter.

FIGS. 89A and 89B illustrate an exemplary method of onboard pooling of 24 samples in accordance with an aspect of the disclosed subject matter.

FIG. 90 illustrates an exemplary pre-treatment process and onboard pooling of pre-treated samples in accordance with an aspect of the disclosed subject matter.

### DETAILED DESCRIPTION

The subject matter disclosed herein is directed to various methods and systems for rapid detection of target nucleic acids in a sample. For example, but not by way of limitation, the subject matter disclosed herein is directed to various methods and systems for rapid screening of donor blood or donor material (e.g., whole blood, lysed whole blood, serum, or plasma) using unique nucleic acid analyses to detect one or more pathogens or infectious agents more efficiently than conventional techniques, and wherein the nucleic acid analyses of the disclosed subject matter is sufficiently sensitive such that determination of a predetermined level of nucleic acid derived from the pathogens or infectious agents is indicative of whether the donor blood or a material derived from the donor (i.e., donor material) can be released for clinical use, such as for blood transfusion, for transplantation or for incorporation into therapeutics.

The methods and systems disclosed herein shift the current process/system of NAT-based analysis of samples, e.g., shift the current process/system of donor blood screening. By eliminating the significant bottlenecks in blood and plasma screening caused by conventional screening assays, the methods and systems of the present disclosure significantly change the donor blood process, simultaneously enhancing efficiency and safety. For example, current NAT-based screening involves assay times that can be hours longer (e.g., 3.5 hours for a time to first result) than serology-based screening and typically require pooling of samples and batching of tests to achieve meaningful throughput, which can create their own inefficiencies in traditional blood screening laboratory operations. In contrast, the methods and systems disclosed herein reduce the time to first result for NAT-based screening from hours to minutes, while simultaneously preserving, or even significantly reducing, instrument size to achieve improvements in overall sample throughput. Importantly, the methods and systems described herein do not merely provide for expedited screening of donor blood, but can reduce the need for blood testing centers to perform sample pooling and restrictive batch processing in order to achieve the necessary throughput to efficiently surveil donor blood.

Beyond the increased throughput afforded by the unique NAT-based screening developments described herein to screen donor blood, the methods and systems of the present disclosure, e.g., the sample preparation, amplification, and detection developments disclosed herein, also allow for highly flexible sample processing. For example, the methods and systems described herein have the ability to interrupt workflows to process priority samples as well as the ability to modify the particular NAT-based screening developments being performed, even after a sample has been prepared and the nucleic acid isolated for amplification. Such flexibility translates into further enhancements to throughput, and provides for reductions in liquid and solid waste by eliminating the inefficient deconstruction of pooled samples. Such flexibility also offers the ability to run “stat” NAT-based screening developments where samples can be processed outside of the batched ordering generally required by current systems.

The enhanced throughput and flexibility of the methods and systems of the present disclosure can increase access to donor blood and blood products by decreasing the time required to process blood, and allow donors to receive more timely information concerning the presence of pathogens or infectious agents.

To achieve the above-described improvements, the present disclosure is directed to, in various embodiments, innovative methods and systems for rapid, sensitive, and high-throughput NAT-based screening of donor blood (e.g., whole blood, lysed whole blood, serum, or plasma) samples that incorporate sample preparation, amplification, or detection aspects, or combinations of such aspects, as disclosed herein.

Reference will now be made in detail to various exemplary embodiments of the disclosed subject matter, which are illustrated in the accompanying drawings and described in greater detail below. The methods of the disclosed subject matter will be described in conjunction with the detailed description of the system. The accompanying drawings, where like reference numerals refer to identical or functionally similar elements throughout the separate views, serve to further illustrate various embodiments and to explain various principles and advantages all in accordance with the disclosed subject matter.

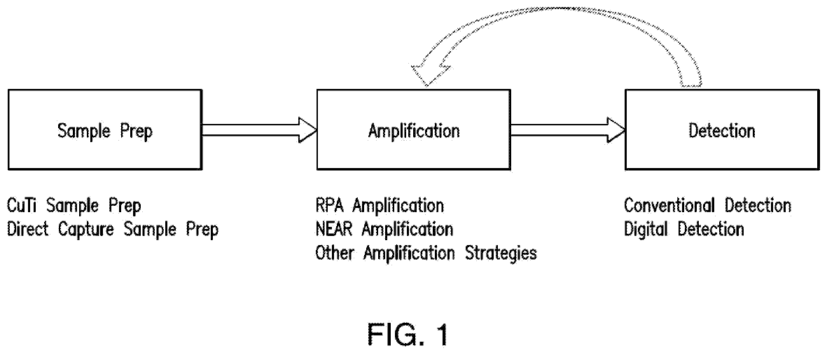

For purpose of introduction, FIG. 1 schematically depicts the general steps of the NAT-based screening of the disclosed subject matter. Particularly, and by illustration and not limitation, FIG. 1 schematically represents the basis for the methods and systems for automated NAT-based screening having improved speed and throughput. In accordance with the disclosed subject matter, for purpose of illustration and not limitation, a system for NAT-based screening is provided in conjunction with the disclosed steps. The system includes a sample preparation component configured to prepare a sample for nucleic acid analysis, an amplification component configured to amplify one or a plurality of targets (e.g., target nucleic acids) in the sample, and a detection component configured to detect a presence or absence of the target or targets (e.g., target nucleic acids) in the sample. In certain embodiments, detection will be performed after amplification is completed. Additionally, or alternatively, the methods and systems embodied by the general steps illustrated in FIG. 1 and described in detail herein can include combined amplification and detection aspects. For example, but not limitation, and as embodied by the dashed arrow in FIG. 1 returning from detection to amplification (the return arrow being dashed to convey its optionality), detection can be repeated, or even continuous, over the course of amplification. Each of these steps and the corresponding components of the disclosed subject matter have various aspects and benefits of the disclosed subject matter will be described in further detail. Furthermore, and as will be described, it is recognized that the various aspects of each step and corresponding components can be selectively combined to achieve the desired benefits of the methods and systems disclosed herein.

With this general understanding, and as described in further detail below, a variety of unique aspects of one or more steps can be incorporated to achieve the desired benefits of the disclosed subject matter. For example, and not limitation, in accordance with one aspect of the disclosed subject matter, the methods and systems of screening donor blood samples can perform a nucleic acid analysis on a sample to detect one or a plurality of pathogens or infectious agents. In certain embodiments, a determination of a predetermined level of nucleic acids derived from each of the pathogens or infectious agents can be based on the nucleic acid analysis performed. The determination can be indicative of whether the donor blood or a donor material can be released for clinical use or further processing. For example, the methods and systems can determine whether the donor blood is acceptable for transfusion based in part on the nucleic acid analysis result. In certain embodiments, the methods and systems can determine whether a material derived from the donor is acceptable for transfusion, transplantation or for production of a therapeutic based in part on the nucleic acid analysis result. In certain embodiments, the detection of a level of a pathogen or infectious agent at or above a predetermined level is indicative that the material derived from the donor is not acceptable for transfusion, transplantation or for production of a therapeutic. In certain embodiments, the presence of a pathogen or infectious agent is indicative that the material derived from the donor is not acceptable for transfusion, transplantation or for production of a therapeutic. In certain embodiments, the detection of a level of a pathogen or infectious agent lower than a predetermined level is indicative that the material derived from the donor is acceptable for transfusion, transplantation or for production of a therapeutic. In certain embodiments, the absence of a pathogen or infectious agent is indicative that the material derived from the donor is acceptable for transfusion, transplantation or for production of a therapeutic. The release of the donor blood or donor material for clinical use can occur in about 15 minutes to about 60 minutes, e.g., about 20 to about 60 minutes, about 20 minutes to about 45 minutes, about 15 minutes to about 45 minutes, about 20 minutes to about 40 minutes or about 15 minutes to about 40 minutes, from initial aspiration of the sample, e.g., from a sample vessel in a sample loading area or from a sample vessel in a sample tube rack at an aspiration position, for performance of the nucleic acid analysis in accordance with the disclosed subject matter.

Additionally, or alternatively, and in accordance with another aspect of the disclosed subject matter, the methods and systems of screening donor blood samples can include performing a nucleic acid analysis on a sample to detect one or a plurality of pathogens or infectious agents. In certain embodiments, the methods and systems can further include the determination of a predetermined level of nucleic acids derived from each of the pathogens or infectious agents based on the nucleic acid analysis performed. In certain embodiments, the methods and systems can further include the determining the presence or absence of nucleic acids derived from each of the pathogens or infectious agents based on the nucleic acid analysis performed. In certain embodiments, the nucleic acid analysis includes a nucleic acid amplification reaction in accordance with the disclosed subject matter that is about 1 minute to about 20 minutes in duration, e.g., about 5 minutes to about 20 minutes or about 8 minutes to about 20 minutes in duration. In certain embodiments, the amplification reaction includes a detection process, e.g., referred to herein as an amplification and detection process. The determination can be indicative of whether the donor blood or a donor material can be released for clinical use. Additionally, the methods and systems can determine whether the donor blood is acceptable for transfusion based in part on the nucleic acid analysis result. In certain embodiments, the methods and systems can determine whether a material derived from the donor is acceptable for transfusion, transplantation or for production of a therapeutic based in part on the nucleic acid analysis result. In certain embodiments, the detection of a level of a pathogen or infectious agent at or above a predetermined level is indicative that the material derived from the donor is not acceptable for transfusion, transplantation or for production of a therapeutic. In certain embodiments, the presence of a pathogen or infectious agent is indicative that the material derived from the donor or donor blood is not acceptable for transfusion, transplantation or for production of a therapeutic. In certain embodiments, the detection of a level of a pathogen or infectious agent lower than a predetermined level is indicative that the material derived from the donor or the donor blood is acceptable for transfusion, transplantation or for production of a therapeutic. In certain embodiments, the absence of a pathogen or infectious agent is indicative that the material derived from the donor or the donor blood is acceptable for transfusion, transplantation or for production of a therapeutic.

Additionally, or alternatively, and in accordance with another aspect of the disclosed subject matter, the methods and systems of screening donor blood samples can perform a nucleic acid analysis on a sample to detect one or a plurality of pathogens or infectious agents, where the determination of a predetermined level of nucleic acids derived from each of the pathogens or infectious agents can be based on the nucleic acid analysis performed, wherein each determination of the predetermined level of nucleic acid from one of the pathogens or infectious agents is completed in about 15 to about 45 minutes, e.g. about 20 to about 45 minutes, about 20 to about 40 minutes or about 15 to about 40 minutes, from initial aspiration of the sample for performance of the nucleic acid analysis in accordance with the disclosed subject matter. The determination can be indicative of whether the donor blood can be released for clinical use. Additionally, the methods and systems can determine whether donor blood is acceptable for transfusion based in part on the nucleic acid analysis result. In certain embodiments, the methods and systems can determine whether a material derived from the donor that provided the donor blood sample can be released for clinical use based in part on the nucleic acid analysis result.

Additionally, or alternatively, and in accordance with another aspect of the disclosed subject matter, the methods and systems of screening donor blood samples can include performing a nucleic acid analysis on a sample to detect one or a plurality of pathogens or infectious agents. In certain embodiments, the methods and system can include the determination of a predetermined level of nucleic acids derived from each of the pathogens or infectious agents based on the nucleic acid analysis performed. In certain embodiments, the methods and systems can include the determination of the presence or absence of nucleic acids derived from each of the pathogens or infectious agents based on the nucleic acid performed. In certain embodiments, the time to result for each determination of the predetermined level of nucleic acid from one of the pathogens or infectious agents is about 15 to about 45 minutes, e.g., about 20 to about 45 minutes, about 20 to about 40 minutes or about 15 minutes to about 40 minutes. The determination can be indicative of whether the donor blood samples can be released for clinical use or a donor material can be released for clinical use. Additionally, the methods and systems can determine whether donor blood is acceptable for transfusion based in part on the nucleic acid analysis result.

Additionally, or alternatively, and in accordance with another aspect of the disclosed subject matter, the methods and systems of screening donor blood samples can perform a nucleic acid analysis on a plurality of samples to detect one or a plurality of pathogens or infectious agents. A determination of a predetermined level of nucleic acids derived from each of the pathogens or infectious agents in each of the plurality of samples can be based on the nucleic acid analyses performed. In certain embodiments, the methods and systems can include the determination of the presence or absence of nucleic acids derived from each of the pathogens or infectious agents based on the nucleic acid performed. The determinations based on the nucleic acid analyses are performed within about 15 minutes to about 3.5 hours, e.g., or about 15 to about 3.5 hours or about 20 minutes to about 3.5 hours, from initial aspiration of the first sample, e.g., from a sample preparation area, for performance of the nucleic acid analysis in accordance with the disclosed subject matter. The determination can be indicative of whether the donor blood can be released for clinical use or a donor material can be released for clinical use. Additionally, the methods and systems can determine whether the donor blood is acceptable for transfusion based in part on the nucleic acid analysis result. In certain embodiments, the methods and systems can determine whether a material derived from the donor can be released for clinical use based in part on the nucleic acid analysis result. For example, but not by way of limitation, the detection of the presence of a pathogen or infectious agent is indicative that the material derived from the donor or donor blood is not acceptable cannot be released for clinical use. In certain embodiments, the absence of a pathogen or infectious agent is indicative that the material derived from the donor or the donor blood can be released for clinical use.

Additionally, or alternatively, and in accordance with another aspect of the disclosed subject matter, the methods and systems of screening donor blood samples can perform a nucleic acid analysis on a plurality of samples to detect one or a plurality of pathogens or infectious agents. In certain embodiments, determinations of a predetermined level of nucleic acids derived from each of the pathogens or infectious agents can be based on the nucleic acid analyses performed. In certain embodiments, methods and systems can perform screening to obtain at least about 70 results per hour per m³ of a volume occupied by the automated system (e.g., used to perform the method), or even at least about 140 results per hour per m² of a footprint of the automated system (e.g., used to perform the method) in accordance with the disclosed subject matter. The determinations can be indicative of whether the donor blood can be released for clinical use or a donor material can be released for clinical use. Additionally, the methods and systems can determine whether the donor blood is acceptable for transfusion based in part on the nucleic acid analysis result. In certain embodiments, the methods and systems can determine whether a material derived from the donor is acceptable for transplantation or for production of a therapeutic based in part on the nucleic acid analysis result.

Additionally, or alternatively, and in accordance with another aspect of the disclosed subject matter, the methods and systems of screening can be used with pooled donor blood samples to perform a nucleic acid analysis on a pooled sample to detect one or a plurality of pathogens or infectious agents. A determination of a predetermined level of nucleic acids derived from each of the pathogens or infectious agents can be based on the nucleic acid analysis performed. Upon a determination of the presence of a nucleic acid derived from at least one of the pathogens or infectious agents, e.g., at or in excess of the predetermined level, in the pooled sample, the methods and systems can screen samples of individual donor blood or sub-pools of donor blood included in the pooled sample, by nucleic acid analyses of the samples to detect one or a plurality of pathogens or infectious agents. Determinations of a predetermined level of nucleic acids derived from each of the pathogens or infectious agents can be based on the nucleic acid analyses performed. The determinations can be indicative of whether the donor blood can be released for clinical use or a donor material can be released for clinical use. Additionally, the methods and systems can determine whether the donor blood is acceptable for transfusion based in part on the nucleic acid analysis result. In certain embodiments, the methods and systems can determine whether a material derived from the donor is acceptable for transplantation or for production of a therapeutic based in part on the nucleic acid analysis result.

Additionally, or alternatively, and in accordance with another aspect of the disclosed subject matter, the methods and systems of screening donor blood samples can include a sample analysis station and a processor and memory with instructions to be executed. The sample analysis station can include a sample loading area, a sample preparation area, a nucleic acid amplification area, and a nucleic acid detection area. When the instructions are executed by the processor, the system can perform a nucleic acid analysis on a sample of donor blood to detect one or a plurality of pathogens or infectious agents. In certain embodiments, upon a determination of a predetermined level of nucleic acids derived from each of the plurality of pathogens or infectious agents based on the nucleic acid analysis, the methods and systems are indicative of release of the donor blood for clinical use or a donor material can be released for clinical use. In certain embodiments, upon determining the absence of the nucleic acids derived from each of the plurality of pathogens or infectious agents based on the nucleic acid analysis, the methods and systems are indicative that the donor blood or donor material can be released for clinical use. Additionally, the methods and systems can determine whether the donor blood is acceptable for transfusion based in part on the nucleic acid analysis result. As embodied herein, the screening of a plurality of donor blood samples for release of the donor blood or a donor material for clinical use and the nucleic acid analysis are performed independently, i.e., without a requirement that the plurality of samples be screened in a predetermined order prior to contact with the sample lysis buffer or that the specific nucleic acid analysis be predetermined prior to contact with the sample lysis buffer. Additionally, or alternatively, such independent nucleic acid analyses allow for distinct analyses to be performed without regard to the samples preceding or following a particular sample, and without impact on the throughput or time to result (TTR) of the samples.

Before describing specific aspects of the methods and systems of the present disclosure in detail below, it is first desirable to describe exemplary methods and systems for use herein. As embodied herein, the exemplary method can be performed on a system including a sample analysis station. For purpose of illustration and not limitation, the sample analysis station can include a sample loading area, a sample preparation area, a nucleic acid amplification area and a nucleic acid detection area. Exemplary methods can include performing a sample preparation process at the sample preparation area. Additionally or alternatively, exemplary methods can include performing an amplification process at the amplification area. Additionally or alternatively, exemplary methods can include performing a detection process at the detection area. For example and not limitation, an exemplary method can include performing a sample preparation process at the sample preparation area, performing an amplification process at the amplification area and performing a detection process at the detection area.

For example and not limitation, the sample preparation area can include a sample transport and a wash and elution system. For example and as embodied herein, the sample preparation area can include a sample transport configured to transport one or more samples in a vessel along a transport path from a sample dispense position to a sample capture and transfer position. For example and as embodied herein, the sample transport can include a sample preparation carousel, e.g., a lysis carousel. For example and as embodied herein, exemplary sample preparation processes can include dispensing a sample into a vessel at the sample dispense position of the sample transport. For example and not limitation, a sample can be dispensed using a pipettor. Exemplary sample preparation processes can further include transporting the sample in a vessel along the transport path of the sample transport to the sample capture and transfer position. For example and not limitation, and as described further herein, exemplary sample preparation processes can include performing a lysis process, pre-treatment process, and/or onboard pooling process on the sample transport of the sample preparation area. As described further herein, exemplary pre-treatment and onboard pooling processes can include transferring samples between vessels on the sample transport as the vessels are transported along the transport path. For example and as embodied herein, the sample preparation area can include a pipettor and the pipettor can transfer, e.g., aspirate and dispense, samples from and into vessels on the sample transport.

Exemplary sample preparation processes can further include transferring the sample from the sample transport to the wash and elution system. For example and as embodied herein, the sample preparation area can include a particle transfer mechanism and the particle transfer mechanism can transfer particles and nucleic acid bound thereto, e.g., CuTi-coated microparticles, to the wash and elution system. As described further herein, exemplary sample preparation processes can include a wash process. Exemplary wash processes can include one or more washing steps, e.g., for washing microparticles bound with nucleic acids, such as CuTi-coated microparticles bound with nucleic acids. Additionally or alternatively, exemplary wash processes can include an elution step. For example and as embodied herein, a wash process can include three wash steps and an elution step.

Additionally or alternatively, exemplary methods can include performing an amplification process at the amplification area and a detection process at the detection area. For purpose of example and as embodied herein the amplification area and detection area can comprise an amplification and detection system and the amplification process and detection processes can include an amplification and detection process. The amplification and detection system can include, for example, a carousel having one or more amplification vessels, and one or more detectors. As embodied herein, a sample can be transferred from the sample preparation area to the amplification and detection area for an amplification and detection process. For example and not limitation, eluate from a wash process can be transferred, e.g., with a pipettor, to the amplification and detection system. Exemplary amplification and detection methods can include, for example, amplifying a target nucleic acid and simultaneously detecting the resulting amplicons, as described further herein.

In certain embodiments, the exemplary method includes performing a nucleic acid analysis on a sample of donor blood, where the nucleic acid analysis includes a sample preparation process and an amplification and detection process. In certain embodiments, the nucleic acid analysis is completed in about 15 minutes to about 34 minutes, where the sample preparation process is completed in about 14 minutes and the amplification and detection process is completed in about 1 to about 20 minutes.

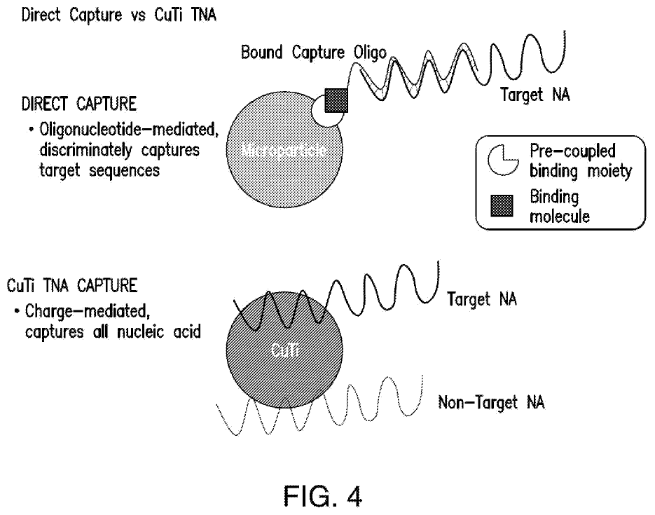

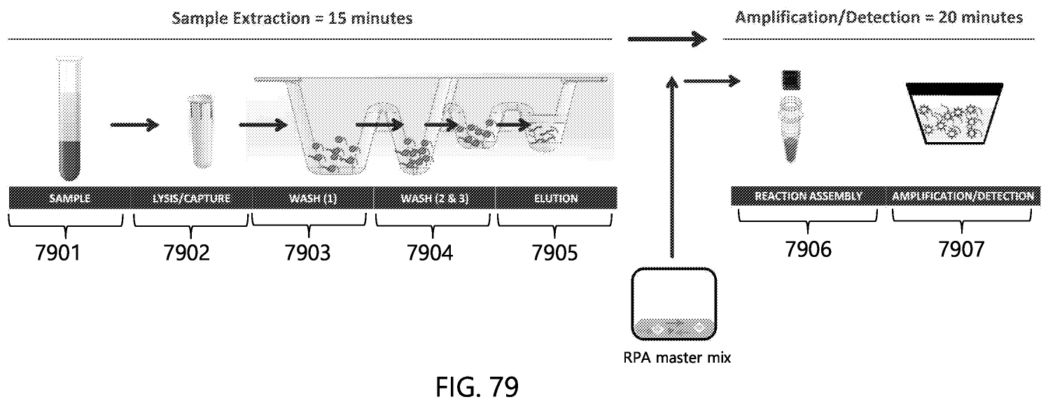

In certain embodiments, the sample preparation process includes providing a sample of donor blood, e.g., a whole blood sample, a plasma sample or a serum sample, as shown in 7901 of FIG. 79, e.g., from a sample loading area. In certain embodiments, the exemplary method can further include a sample preparation process. For example and as shown in 7902 of FIG. 79, the sample preparation process can include a lysis process, e.g. lysing the sample in a lysis vessel. The lysis process can be performed on a sample transport, e.g., a sample preparation carousel such as for example lysis carousel 6805 or 8001. In certain embodiments, the lysis process can include combining the sample with a lysis buffer, microparticles, e.g., CuTi-coated microparticles, and Proteinase K to generate a mixture. In certain embodiments, the lysis process and lysis of the sample does not require a separate Proteinase K treatment step. In certain embodiments, the lysis process, e.g., lysing the sample, can further include combining the mixture with internal control nucleic acids or calibrators. In certain embodiments, the exemplary method can further include incubating the mixture to promote binding of nucleic acids within the mixture to the microparticles. As shown in FIG. 4, CuTi microparticles can bind both RNA and DNA in the sample including target nucleic acids (e.g., pathogenic nucleic acids) and non-target nucleic acids (e.g., host nucleic acids).

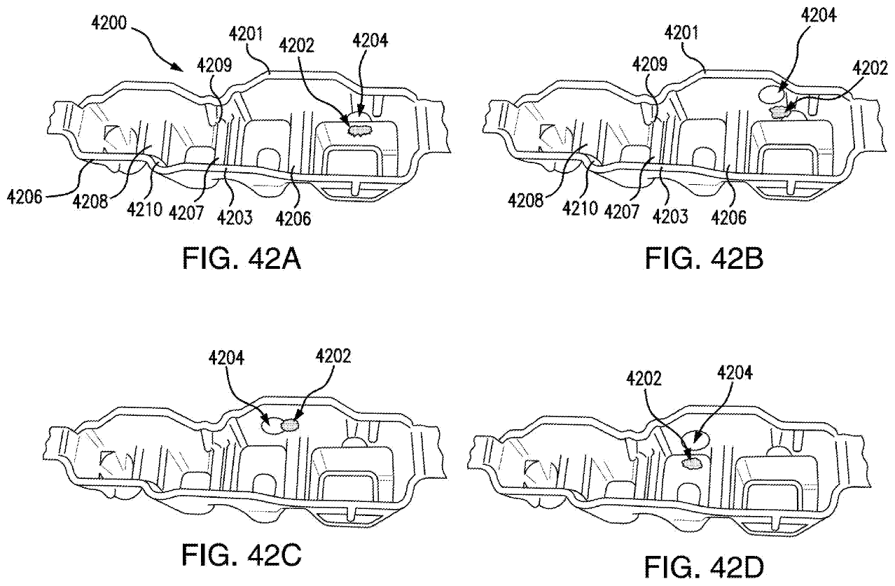

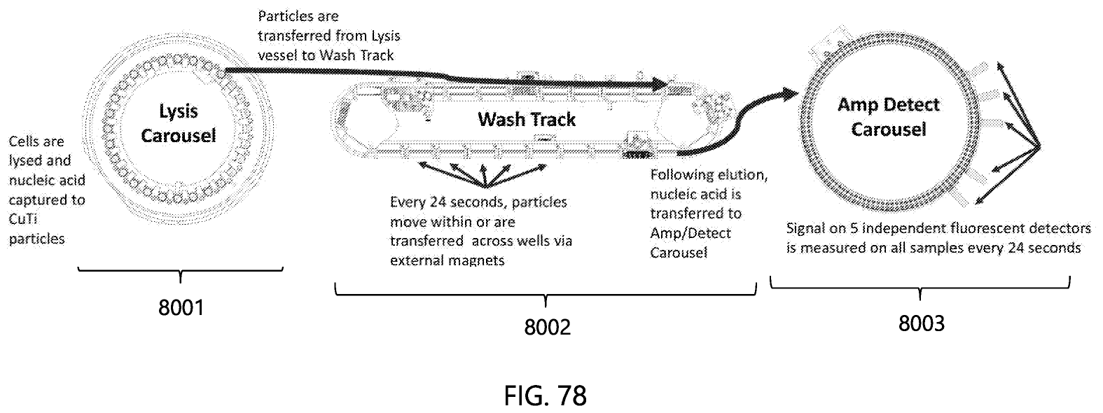

In certain embodiments, the sample preparation process of the exemplary method can include a wash process. In certain embodiments, the wash process can include washing the microparticles bound with nucleic acids with a first wash. For example, the wash process can include a first wash as shown in 7903 in FIG. 79. In certain embodiments, the first wash can include lysis buffer. In certain embodiments, the microparticles bound with nucleic acids can be transferred from the sample transport, e.g., from a lysis vessel, to a different vessel to perform the first wash. For example, microparticles bound with nucleic acids can be transferred from the sample transport to a wash and elution system. For example, but not by way of limitation, the microparticles can be transferred from the lysis vessels in the sample transport, e.g., lysis carousel 6805 or 8001 to the wash and elution system, e.g., to a wash vessel of a wash track 6801 or 8002 using a particle transfer mechanism 6803. In certain embodiments, a wash vessel for use in the present disclosure has the structure shown in FIGS. 42, 71 and 79. For example, but not by way of limitation, a wash vessel for use in the present disclosure can include more than one (1) well, e.g., four (4) wells. As embodied herein, the wash and elution system can include a wash track, e.g. wash track 6801 or 8002. The wash track of the disclosed system can be configured to perform wash steps of the disclosed method and can further include a plurality of wash vessels. For purpose of illustration not limitation, the wash track, e.g., wash track 6801 or 8002 can be in the shape of a racetrack. In certain embodiments, the wash process of the exemplary method can subsequently include washing the microparticles bound with nucleic acids with water two times, e.g., a second wash with water and a third wash with water, e.g. as shown in 7904 of FIG. 79. In certain embodiments, the second and third washes are performed in wells different from the first wash, e.g., by applying a magnetic force to capture the microparticles on an inner surface of a first wall of the first well and translating the captured microparticles along the inner surface of the first wall to the second/third well. For purpose of illustration and not limitation, the particles can be moved within or are transferred across wells in about 24 seconds, as shown in FIG. 78. In certain embodiments, the nucleic acids bound to the microparticles can be eluted (e.g., by using an elution buffer and/or by heat) to generate an eluate in a fourth well of the wash vessel, e.g., as depicted in 7905 of FIG. 79.

In certain embodiments, the exemplary method further includes performing an amplification and detection process. In certain embodiments, amplification and detection are performed simultaneously. In certain embodiments, the amplification and detection process of the exemplary method include preparing the eluate for an isothermal amplification reaction to amplify a target nucleic acid of interest in the eluate and simultaneous detection of the amplified target nucleic acid, e.g., as shown in 7906 of FIG. 79. As embodied herein, following elution, the target nucleic acid can be transferred to an amplification and detection system 6807. For purpose of illustration and not limitation, the amplification and detection system can include a carousel. For example, the amplification and detection system 6807 includes a carousel 8003. In certain embodiments, preparing the eluate for amplification and detection includes contacting the eluate with a reagent mixture and an activator, e.g., magnesium. In certain embodiments, the reagent mixture can be, as referred to herein, a “RPA master mix”, e.g., as shown in FIG. 79. In certain embodiments, the RPA master mix includes seven (7) enzymes that facilitate primer binding and extension in the amplification reaction, e.g., (i) a recombinase (e.g., UvsX), (ii) a single strand binding protein (e.g., GP32), (iii) a recombinase loading agent (e.g., UvsY), (iv) a DNA polymerase, (v) an exonuclease (e.g., Exonuclease III), (vi) Creatine Kinase and (vii) a reverse transcriptase (e.g., EIAV-RT). In certain embodiments, the reverse transcriptase is not included in the RPA master mix, e.g., if the target nucleic acid is DNA. In certain embodiments, the RPA master mix can further include one or more primers that bind to the target nucleic acid, one or more probes that bind to the amplicon to facilitate signal generation. In certain embodiments, the RPA master mix can further include one or more non-protein components (e.g., for extending the primers (e.g., dNTPs), for use as an energy source (e.g., ATP and phosphocreatine), for stabilizing the proteins and reaction (e.g. reaction buffer (e.g., Tris and salts)) and for use as a crowding agent (e.g., polyethylene glycol)).

In certain embodiments, the exemplary method can include amplifying the target nucleic acid of interest using an isothermal amplification reaction and simultaneously detecting the resulting amplicons, e.g., by fluorescent detection. For example, but not by way of limitation, the exemplary method can include amplifying the target nucleic acid of interest using an isothermal amplification reaction and simultaneously detecting the resulting amplicons as depicted in 7907 of FIG. 79. Non-limiting examples of isothermal amplification reactions can include transcription-mediated amplification (TMA), Recombinase-Polymerase Amplification (RPA) and Nicking Enzyme Amplification Reaction (NEAR). In certain embodiments, the isothermal amplification reaction is RPA. The use of isothermal amplification obviates the need for time consuming temperature transitions. As embodied herein, the amplification and detection system can include independent fluorescent detectors, e.g., about 5 independent fluorescent detectors, to detect fluorescent signals at a predetermined interval of about every 24 seconds, e.g., during the amplification reaction. For example, but not by way of limitation, the amplification and detection system 6807 can include about 5 independent fluorescent detectors to detect fluorescent signals at a predetermined interval of about every 24 seconds as shown in FIG. 78. In certain embodiments, the exemplary method can further include determining a result from the nucleic acid analysis, e.g., determining the presence of a nucleic acid derived from at least one of a plurality of pathogens or infectious agents at or in excess of the predetermined level in the donor blood sample, determining the presence of a nucleic acid derived from at least one of a plurality of pathogens or infectious agents in an amount less than the predetermined level in the donor blood sample, determining the presence of a nucleic acid derived from at least one of a plurality of pathogens or infectious agents in the donor blood sample or determining the absence of a nucleic acid derived from at least one of a plurality of pathogens or infectious agents in the donor blood sample.

In certain embodiments, the exemplary methods and systems of the present disclosure can be used to perform a nucleic acid analysis on a sample. In certain embodiments, the exemplary methods and systems of the present disclosure can be used to screen individual or pooled blood donors (e.g., whole blood, lysed whole blood, serum, or plasma), e.g., to determine whether the donor samples are acceptable for transfusion, and to screen organ and/or tissue donors for determining whether a material derived from the donor (i.e., a donor material) is acceptable for clinical use. In certain embodiments, the exemplary methods and systems of the present disclosure can be used for quantitative and/or qualitative detection of nucleic acids derived from a pathogen or infectious agent in a sample, e.g., a sample of donor blood. Additional examples of samples to be analyzed using the disclosed methods and systems are described herein. In certain embodiments, the exemplary methods and systems of the present disclosure can be used for qualitative detection of nucleic acids derived from a pathogen or infectious agent in a sample, e.g., a sample of donor blood. For example, but not by way of limitation, the exemplary methods and systems of the present disclosure can be used to determine the presence or absence of nucleic acids derived from a pathogen or infectious agent in a sample, e.g., a sample of donor blood. In certain embodiments, the exemplary methods and systems of the present disclosure can be used for quantitative detection of nucleic acids derived from a pathogen or infectious agent in a sample, e.g., a sample of donor blood. In certain embodiments, the exemplary methods and systems of the present disclosure can be used to detect and/or quantify nucleic acids derived from pathogens including but not limited to HIV-1, HIV-2, HCV, HBV, WNV, Zika Virus, Chikungunya Virus, Dengue Virus, Babesia, Malaria (e.g., by detecting and/or quantifying the Plasmodium that causes Malaria), Parvovirus B19, HAV and/or HEV. In certain embodiments, the exemplary methods and systems of the present disclosure can be used for qualitative detection of nucleic acids derived from one or more pathogens or infectious agents. In certain embodiments, the exemplary methods and systems of the present disclosure can be used for qualitative detection of nucleic acids derived from HIV-1, HIV-2, HBV, HCV, Parvovirus B19, HAV, WNV, Zika Virus, Dengue Virus, Chikungunya Virus, Babesia, Malaria, Usutu Virus and/or HEV. In certain embodiments, the exemplary methods and systems of the present disclosure can be used for qualitative detection of nucleic acids derived from SARS-CoV-2 (COVID-19), coronaviruses, HIV-1, HIV-2, HBV, HCV, CMV, Epstein-Barr virus (EBV), human T-lymphotropic virus (HTLV), Parvovirus B19, HAV, syphilis, Chlamydia, Gonorrhea, Dengue, Chikungunya, WNV, HEV, Usutu virus and/or Creutzfeldt-Jakob disease (vCJD). In certain embodiments, the exemplary methods and systems of the present disclosure can be used for qualitative detection of nucleic acids derived from HIV-1, HIV-2, HCV, HBV and WNV in serum or plasma samples. In certain embodiments, the exemplary methods and systems of the present disclosure can be used for multiplex analysis of HIV-1, HIV-2, HCV and HBV in serum or plasma samples. In certain embodiments, the exemplary methods and systems of the present disclosure can be used for qualitative detection of nucleic acids derived from Babesia in whole blood samples. In certain embodiments, the exemplary methods and systems of the present disclosure can be used for qualitative detection of nucleic acids derived from HAV in plasma samples. In certain embodiments, the exemplary methods and systems of the present disclosure can be used for quantitative detection of nucleic acids derived from Parvovirus B19 in plasma samples.

In certain embodiments, the exemplary methods and systems of the present disclosure can be used for quantitative detection of nucleic acids derived from one or more pathogens or infectious agents disclosed herein. For example, but not by way of limitation, the exemplary methods and systems of the present disclosure can be used for quantitative detection of nucleic acids derived from Parvovirus B19.

In certain embodiments, an exemplary method of the present disclosure can be completed in about 15 minutes to about 60 minutes using a system disclosed herein, e.g., about 15 minutes to about 60 minutes from initial aspiration of the sample for lysis from a sample vessel, e.g., a sample vessel present in a sample loading area or a sample vessel in a sample tube rack at an aspiration position. In certain embodiments, an exemplary method of the present disclosure can be completed in about 15 minutes to about 45 minutes using a system disclosed herein, about 15 minutes to about 40 minutes using a system disclosed herein or about 15 minutes to about 34 minutes using a system disclosed herein, e.g., about 15 minutes to about 45 minutes from aspiration of the sample for lysis from a sample vessel in a sample tube rack at an aspiration position or from a sample loading area. In certain embodiments, an exemplary method of the present disclosure can be completed in about 20 minutes to about 60 minutes using a system disclosed herein, e.g., about 20 minutes to about 60 minutes from initial aspiration of the sample for lysis. In certain embodiments, an exemplary method of the present disclosure can be completed in about 20 minutes to about 45 minutes using a system disclosed herein, e.g., about 20 minutes to about 45 minutes or about 20 minutes to about 34 minutes from initial aspiration of the sample for lysis from a sample vessel, e.g., a sample vessel in a sample tube rack at an aspiration position or a sample vessel present in a sample loading area. For example, but not by way of limitation, an exemplary method of the present disclosure can be completed to produce a result, e.g., determining an amount of target nucleic acid amplified in the amplification reaction, in about 35 minutes or less from initial aspiration of the sample for lysis using a system disclosed herein.

In certain embodiments, the nucleic acid analysis starts with the aspiration of a sample from a sample vessel, e.g., in a sample loading area or in a sample tube rack at an aspiration position, and ends with the determination of a result. In certain embodiments, the nucleic acid analysis starts with the aspiration of a sample from a sample vessel, e.g., in a sample loading area or in a sample tube rack at an aspiration position, and ends at the end of the incubation of the sample in an amplification vessel on the amplification and detection system. In certain embodiments, the nucleic acid analysis can be completed in about 15 minutes to about 36 minutes, about 16 minutes to about 36 minutes, about 17 minutes to about 36 minutes, about 18 minutes to about 36 minutes, about 19 minutes to about 36 minutes, about 20 minutes to about 30 minutes, about 33 minutes to about 35 minutes, or about 32 minutes to about 36 minutes. In certain embodiments, the nucleic acid analysis can be completed in about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, or about 45 minutes. In certain embodiments, the nucleic acid analysis can be completed in about 15 minutes. In certain embodiments, the nucleic acid analysis can be completed in about 34 minutes. In certain embodiments, exemplary methods and systems of the present disclosure can be used to obtain at least about 150 to about 300 results per hour. In certain embodiments, exemplary methods and systems of the present disclosure can be used to obtain at least about 1,000 to about 2,500 results per 8 hour period, e.g., about 1,100 to about 2,300 results per 8 hour period. In certain embodiments, exemplary methods and systems of the present disclosure can be used to obtain at least about 500 to about 1,200 results per 8 hours per m², e.g., at least about 570 to about 1,150 results per 8 hours per m² of a footprint of the automated system.

For clarity, but not by way of limitation, the detailed description of the presently disclosed subject matter is divided into the following subsections:
1. Sample Collection Aspects;
2. Sample Preparation Aspects;
3. Nucleic Acid Amplification Aspects;
4. Nucleic Acid Detection Aspects;
5. Methods of Use;
6. Analytical Systems;
7. Additional Screening and Preparation Aspects; and
8. Exemplary Embodiments.

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein the term “donor” refers to any animal (e.g., a mammal), including, but not limited to, humans, non-human primates, rodents, and the like, which donates a biological sample. For example, but not by way of limitation, the biological sample can be blood, serum, or plasma, e.g., for use in transfusions.

As used herein “donor blood” refers to blood obtained from a donor, e.g., whole blood, lysed whole blood, serum, or plasma, as well as products derived from such blood, e.g., platelets, packed red blood cells, and plasma-derived products such as, but not limited to: (1) coagulation factors, e.g., factor VIII, von Willebrand factor, and fibrinogen; (2) protease inhibitors, e.g., alpha1-antitrypsin and C1-esterase inhibitor; (3) albumin; and (4) immunoglobulin G (IgG).

As used herein the term “patient” refers to any animal (e.g., a mammal), including, but not limited to, humans, non-human primates, rodents, and the like, which is to be recipient of a particular clinical treatment, e.g., a transfusion.

As used herein, the phrase “clinical use” refers to “in vivo clinical uses” and “in vitro clinical uses.” As used herein, “in vivo clinical use” refers to transfusions of whole blood as well as the transfusion of components of whole blood, e.g., packed red blood cells, plasma (e.g., fresh frozen plasma or thawed plasma), platelets, or cryoprecipitate (which is prepared by thawing fresh frozen plasma and collecting the precipitate), collectively referred herein as “blood products.” “In vivo clinical use” also encompasses the incorporation of donor blood, or materials derived therefrom, in the production of therapeutics and the donation and/or transplantation of one or more materials, e.g., organs, tissues, etc., from a donor. For example, but not limitation, donor blood can be processed into plasma, either after collection as whole blood or directly as a plasma donation via automated apheresis methods where blood is removed from a donor, the plasma is collected, and the remaining blood is returned to the donor, and that plasma can either be used directly, as fresh frozen plasma or it can be further processed to produce a variety of therapeutic biologics known as plasma-derived products. For example, but not limitation, plasma can be pooled, typically to a significant degree, e.g., pools of 10,000 to 50,000 donations are combined for industrial processing, and the pooled plasma can be fractionated to produce a variety of plasma-derived products including, but not limited to: (1) coagulation factors, e.g., factor VIII, von Willebrand factor, and fibrinogen; (2) protease inhibitors, e.g., alpha1-antitrypsin and C1-esterase inhibitor; (3) albumin; and (4) immunoglobulin G (IgG). As used herein, “in vitro clinical use” refers to the use of biological materials outside of the direct transfusion or transplantation of blood products, plasma-derived products or donor materials into patients, e.g., in research and development of new medical devices, therapeutic processes, or disease diagnostics, as well as in connection with quality assurance/laboratory diagnostics.

As used herein, the phrase “donor material” refers to blood products and other biological products, including, for example, tissues, organs, vaccines, cells, gene therapy, and recombinant therapeutic proteins. Donor material can include more than one donor material. For example, donor material can include more than one blood product and/or other biological product from a single donor. Additionally or alternatively, donor material can include blood products from multiple donors, biological products from multiple donors, or blood products from one donor and other biological products from one or more other donors.

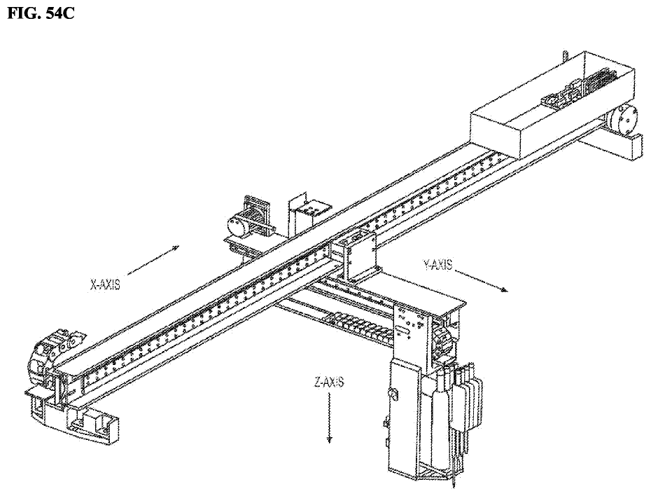

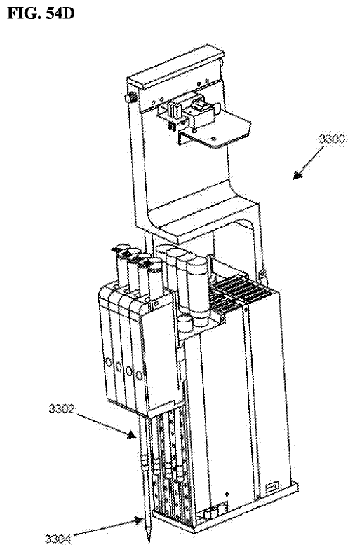

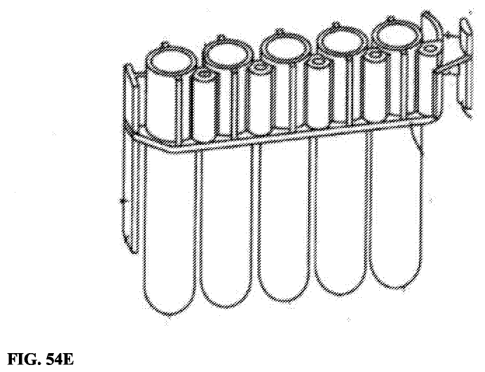

As used herein, the phrase time to result (“TTR”) refers to the time from initiation of a nucleic acid analysis comprising, for example, sample preparation, amplification, and detection, to the completion of the detection step of the nucleic acid analysis. In certain embodiments, the initiation of a nucleic acid analysis occurs at the initial aspiration of the sample, e.g., from a sample vessel at the sample loading area or from a sample vessel in a sample tube rack at an aspiration position, for performance of the nucleic acid analysis in accordance with the disclosed subject matter. In certain embodiments, the sample vessel includes a vessel in the sample loading area. For example, in certain embodiments, aspiration of samples for nucleic acid analysis can occur at an aspiration position, e.g., aspiration position 6292. Additionally or alternatively, aspiration of samples for nucleic acid analysis can occur at an internal portion of the sample loading area, e.g., the sample loading area 3102 of FIG. 54B. In certain embodiments, TTR refers to the time from initiation of a nucleic acid analysis, e.g., from initial aspiration of the sample from a sample loading area or from a sample vessel in a sample tube rack at an aspiration position, to the time a result is obtained during the amplification and detection process. In certain embodiments, TTR refers to the time from initiation of a nucleic acid analysis, e.g., from initial aspiration of the sample from a sample loading area or from a sample vessel in a sample tube rack at an aspiration position, to the completion of the amplification and detection process.

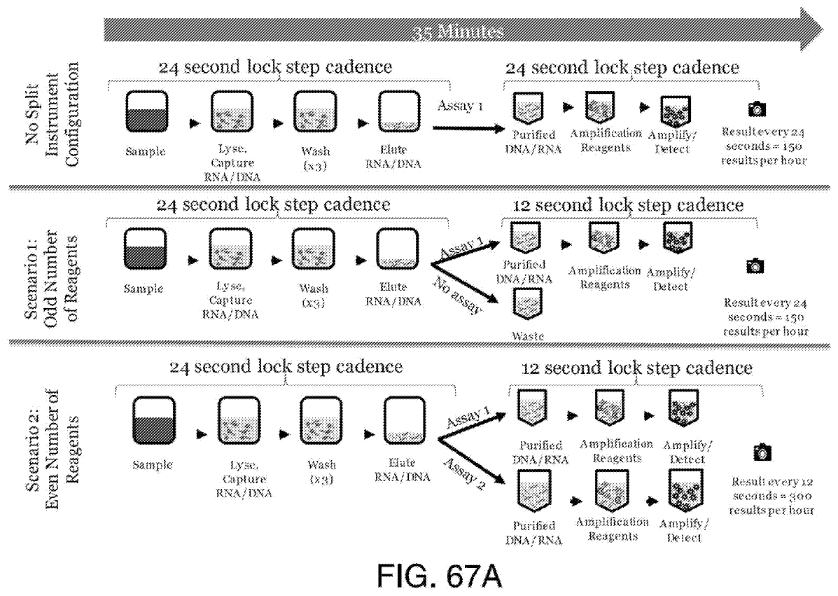

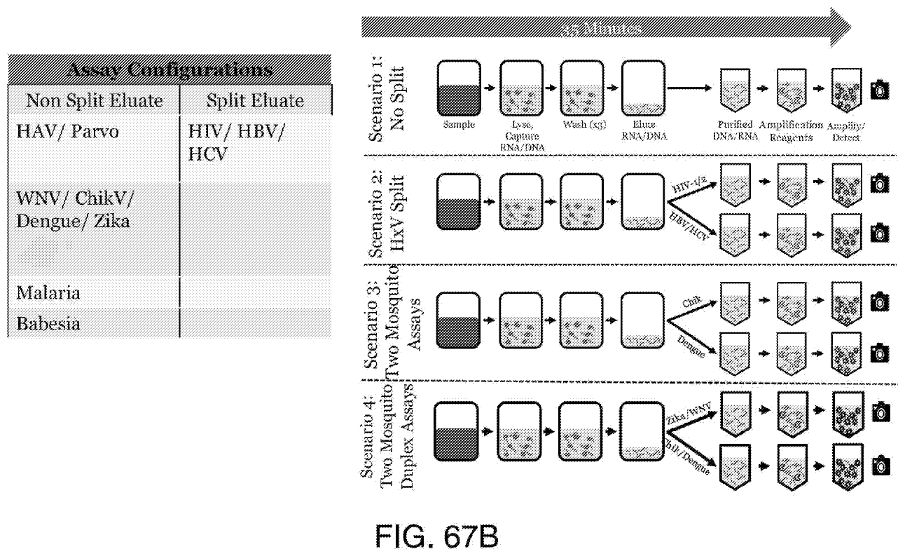

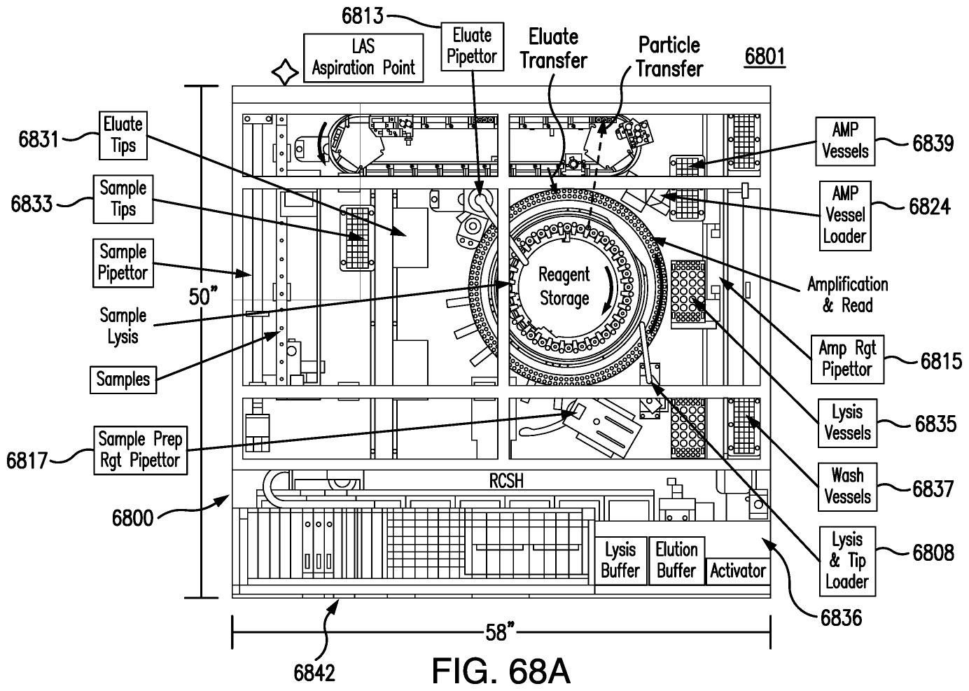

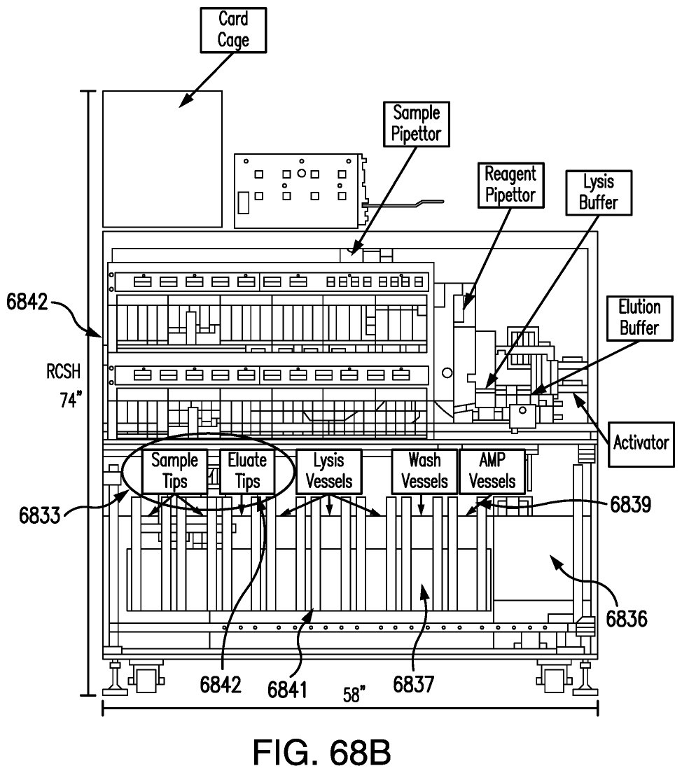

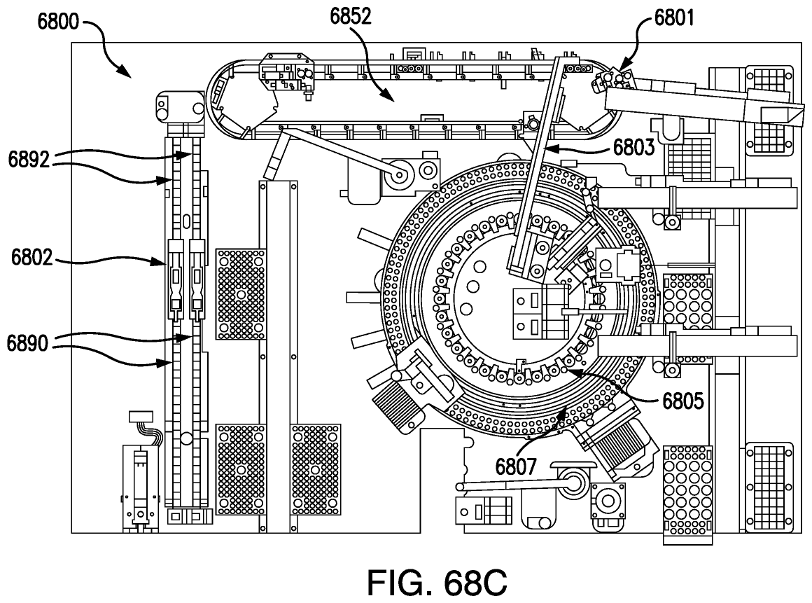

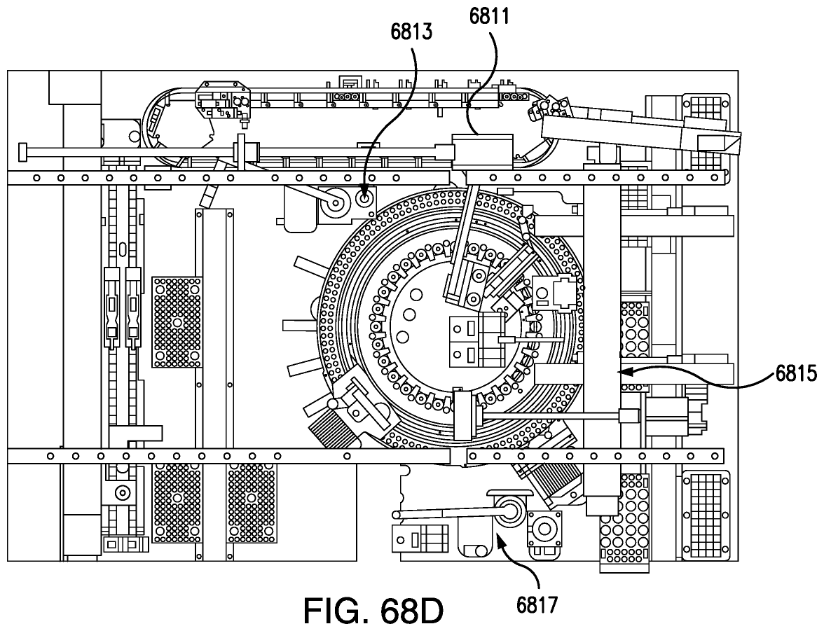

A “result,” as used herein, refers to the detection of the presence of one or more target nucleic acids. In certain embodiments, a result obtained using the methods and systems of the present disclosure can include determining the absence of one or more target nucleic acids. In certain embodiments, a result obtained using the methods and systems of the present disclosure can include the detection of one or more target nucleic acids at or in excess of a predetermined level. In certain embodiments, a result obtained using the methods and systems of the present disclosure can include the detection of one or more target nucleic acids at an amount lower than a predetermined level. In certain embodiments, a result obtained using the methods and systems of the present disclosure can include the quantification of one or more target nucleic acids. In certain embodiments, as embodied in FIGS. 67A and 67B, a single sample aspiration, can, in certain embodiments, result in an eluate dispensed into two amplification reactions (e.g., referred to herein as a “split eluate”), thereby producing two or more “results.” As used herein, a result can comprise the detection of, e.g., the detection of the presence or absence of, one or a plurality of target nucleic acids. For example, but not limitation, Scenarios 2 and 4 of FIG. 67B each illustrate the use of a single sample preparation process followed by two amplification reactions wherein those amplifications are multiplexed to detect two target nucleic acids, each derived from a different pathogen or infectious agent. Each of these Scenarios illustrates obtaining four results, as each amplification reaction provides information with respect to two target nucleic acids, such that a single sample can, in those Scenarios, allow for the detection of the presence or absence of four pathogens or infectious agents with the two amplification reactions. Additionally, or alternatively, higher orders of multiplex amplifications can be employed in connection with the methods and systems of the present disclosure, such that the presence of 5, 6, 7, 8, 9, 10 or more pathogens or infectious agents can be detected in a single sample, thereby providing 5, 6, 7, 8, 9, 10 or more results, respectively. In certain embodiments, each result comprises a discernible detection of the presence, absence or level of a target nucleic acid.

Because TTR, as used herein, refers to the time from initiation of a nucleic acid analysis, it necessarily excludes any time occurring between the donation and its screening by an HTNAT analysis system or method as described herein. For example, but not limitation, time spent sampling, e.g., drawing of the donor blood, or sample transportation time, e.g., transportation of samples from a local blood collection center or plasma center to a central laboratory for screening, is excluded from TTR.

As used herein, the term “throughput” refers the number of nucleic acid analysis results obtained per unit time, e.g., per hour. Additionally or alternatively, throughput can refer, without limitation, to the number of samples analyzed per unit time, e.g., per hour. In certain embodiments, throughput can refer to, without limitation, the number of tests run per unit of time, e.g., per hour.

As used herein, the use of the word “a” or “an” when used in conjunction with the term “comprising” in the claims and/or the specification can mean “one,” but it is also consistent with the meaning of “one or more,” “at least one,” and “one or more than one.”

The term “certain” as used, for example, with “certain embodiments” and “certain aspects” is a reference to “exemplary” and is not limiting.

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “expression” or “expresses,” as used herein, refer to transcription and translation occurring within a cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

The term “nucleic acid,” “nucleic acid molecule” or “polynucleotide” as used herein refers to any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby the bases represent the primary structure (linear structure) of a nucleic acid molecule. The sequence of bases is typically represented from 5′ to 3′. Herein, the term nucleic acid molecule encompasses deoxyribonucleic acid (DNA) including, e.g. complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues.

The term “oligonucleotide,” as used herein, refers to a short nucleic acid sequence comprising from about 2 to about 100 nucleotides (e.g., about 5, 10, 15, 20, 25, 30, 35, 40, 45, 50, 55, 60, 65, 70, 75, 80, 85, 90, 95, 99, or 100 nucleotides, or a range defined by any of the foregoing values). The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, for example, methylated and/or capped polynucleotides. Nucleic acids are typically linked via phosphate bonds to form nucleic acid sequences or polynucleotides, though many other linkages are known in the art (e.g., phosphorothioates, boranophosphates, and the like).

Oligonucleotides can be single-stranded or double-stranded or can contain portions of both double-stranded and single-stranded sequences. The oligonucleotide can be DNA, both genomic and complimentary DNA (cDNA), RNA, or a hybrid, where the nucleic acid can contain combinations of deoxyribo- and ribonucleotides, and combinations of bases including uracil, adenine, thymine, cytosine, guanine, inosine, xanthine hypoxanthine, isocytosine and isoguanine. Oligonucleotides can be obtained by chemical synthesis methods or by recombinant methods.

Any of the oligonucleotides described herein can be modified in any suitable manner so as to stabilize or enhance the binding affinity of the oligonucleotide for its target. For example, an oligonucleotide sequence as described herein can comprise one or more modified oligonucleotide bases.

Any of the oligonucleotide sequences described herein can comprise, consist essentially of, or consist of a complement of any of the sequences disclosed herein. The terms “complement” or “complementary sequence,” as used herein, refer to a nucleic acid sequence that forms a stable duplex with an oligonucleotide described herein via Watson-Crick base pairing rules, and typically shares about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% greater identity with the disclosed oligonucleotide.

The oligonucleotides described herein can be prepared using any suitable method, a variety of which are known in the art (see, for example, Sambrook et al., Molecular Cloning. A Laboratory Manual, 1989, 2. Supp. Ed., Cold Spring Harbour Laboratory Press: New York, N.Y.; M. A. Innis (Ed.), PCR Protocols. A Guide to Methods and Applications, Academic Press: New York, N.Y. (1990); P. Tijssen, Hybridization with Nucleic Acid Probes—Laboratory Techniques in Biochemistry and Molecular Biology (Parts I and II), Elsevier Science (1993); M. A. Innis (Ed.), PCR Strategies, Academic Press: New York, N.Y. (1995); and F. M. Ausubel (Ed.), Short Protocols in Molecular Biology, John Wiley & Sons: Secaucus, N.J. (2002); Narang et al., Meth. Enzymol., 68: 90-98 (1979); Brown et al., Meth. Enzymol., 68: 109-151 (1979); and Belousov et al., Nucleic Acids Res., 25: 3440-3444 (1997), each of which is incorporated herein by reference in its entirety). Oligonucleotide pairs also can be designed using a variety of tools, such as the Primer-BLAST tool provided by the National Center of Biotechnology Information (NCBI). Oligonucleotide synthesis can be performed on oligo synthesizers such as those commercially available from Perkin Elmer/Applied Biosystems, Inc. (Foster City, CA), DuPont (Wilmington, DE), or Milligen (Bedford, MA). Alternatively, oligonucleotides can be custom made and obtained from a variety of commercial sources well-known in the art, including, for example, the Midland Certified Reagent Company (Midland, TX), Eurofins Scientific (Louisville, KY), BioSearch Technologies, Inc. (Novato, CA), and the like. Oligonucleotides can be purified using any suitable method known in the art, such as, for example, native acrylamide gel electrophoresis, anion-exchange HPLC (see, e.g., Pearson et al., J Chrom., 255: 137-149 (1983), incorporated herein by reference), and reverse phase HPLC (see, e.g., McFarland et al., Nucleic Acids Res., 7: 1067-1080 (1979), incorporated herein by reference).

The sequence of the oligonucleotides can be verified using any suitable sequencing method known in the art, including, but not limited to, chemical degradation (see, e.g., Maxam et al., Methods of Enzymology, 65: 499-560 (1980), incorporated herein by reference), matrix-assisted laser desorption ionization time-of-flight (MALDI-TOF) mass spectrometry (see, e.g., Pieles et al., Nucleic Acids Res., 21: 3191-3196 (1993), incorporated herein by reference), mass spectrometry following a combination of alkaline phosphatase and exonuclease digestions (Wu et al., Anal. Biochem., 290: 347-352 (2001), incorporated herein by reference), and the like.

The terms “primer,” “primer sequence,” “primer oligonucleotide,” and “amplification oligonucleotide” as used herein, refer to an oligonucleotide which is capable of acting as a point of initiation of synthesis of an extension product that is a complementary strand of nucleic acid (all types of DNA or RNA) when placed under suitable amplification conditions (e.g., buffer, salt, temperature and pH) in the presence of nucleotides and an agent for nucleic acid polymerization (e.g., a DNA-dependent or RNA-dependent polymerase). The amplification oligonucleotides of the present disclosure can be of any suitable size, and desirably comprise, consist essentially of, or consist of about 15 to 50 nucleotides, preferably about 20 to 40 nucleotides. The oligonucleotides of the present disclosure can contain additional nucleotides in addition to those described herein.

The terms “probe,” “probe sequence,” and “probe oligonucleotide,” refer to an oligonucleotide that can selectively hybridize to at least a portion of a target sequence (e.g., a portion of a target sequence that has been amplified) under appropriate hybridization conditions. In general, a probe sequence is identified as being either “complementary” (i.e., complementary to the coding or sense strand (+)), or “reverse complementary” (i.e., complementary to the anti-sense strand (−)). The probes of the present disclosure can be of any suitable size, and desirably comprise, consist essentially of, or consist of about 10-50 nucleotides, preferably about 12-35 nucleotides.

As used herein, the terms “set,” “primer set,” “probe set,” and “primer and probe set,” refer to two or more oligonucleotides which together are capable of priming the amplification of a target sequence or target nucleic acid of interest (e.g., a target sequence within an infectious agent) and/or at least one probe which can detect the target sequence or target nucleic acid. In certain embodiments, the term “set” refers to a pair of oligonucleotides including a first oligonucleotide, referred herein as a “forward primer” that hybridizes with the 5′-end of the target sequence or target nucleic acid to be amplified and a second oligonucleotide, referred herein as a “reverse primer” that hybridizes with the complement of the target sequence or target nucleic acid to be amplified.

The terms “target nucleic acid”, “target sequence”, or “target nucleic acid sequence,” as used herein, refers to a nucleic acid sequence of a pathogen or infectious agent, such as a virus, bacteria or eukaryotic parasite described herein, or a complement thereof. In certain embodiments, a target sequence or target nucleic acid sequence can be detected using the methods and systems of the present disclosure.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, “percentage of sequence identity” or “percentage of identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons. Sequence alignment algorithms also are disclosed in, for example, Altschul et al., J. Molecular Biol., 215(3): 403-410 (1990); Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009); Soding, Bioinformatics, 21(7): 951-960 (2005); Altschul et al., Nucleic Acids Res., 25(17): 3389-3402 (1997); and Gusfield, Algorithms on Strings, Trees and Sequences, Cambridge University Press, Cambridge UK (1997), each of which is incorporated herein by reference in its entirety).

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

As used herein, the term “amplified” refers to the process of making multiple copies of the nucleic acid from a single or lower copy number of nucleic acid sequence molecule. The amplified nucleic acid can be referred to as an amplicon.

The terms “detect” or “detection,” as used herein, indicates the determination of the existence and/or presence of a target nucleic acid in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of the presence or absence of a target or signal, without dependence on the quantity or amount of the target or signal beyond its presence or absence.

As used herein, the term “pathogen reduction technology” refers to techniques, strategies and/or technologies for reducing, eliminating and/or inactivating a pathogen that can be present in a sample. Several pathogen reduction technology methods have been developed, including solvent/detergent treatment, light treatment (with or without a photosensitizer) and chemical treatment.

As used herein, the term “initial aspiration” is a broad term, and is to be given its ordinary and customary meaning to a person of skill in the art (and it is not to be limited to a special or customized meaning), and can refer without limitation to the first aspiration of a sample or portion thereof from a sample tube or sample vessel for performance of nucleic acid analysis on the sample or portion thereof. For example and not limitation, and as described further herein, an initial aspiration can include aspirating a sample or portion thereof from a sample tube with a pipettor. For purpose of example and as embodied herein, initial aspiration can include aspirating a sample or portion thereof from a sample tube or sample vessel in a sample tube rack at an aspiration position within an automated system for screening a sample of donor blood for release of donor material for clinical use. Additionally or alternatively, for example and not limitation, and as further embodied herein, an initial aspiration can include aspirating a sample from a sample loading area of an automated system for screening a sample of donor blood for release of donor material for clinical use. For example and not limitation, after initial aspiration, nucleic acid analysis can be performed on the sample or portion thereof. For example and not limitation and as described further herein, after initial aspiration, the aspirated sample or portion thereof can be transferred to a sample preparation area for a sample preparation process, e.g., the sample can be dispensed into a lysis tube on a sample preparation carousel for a lysis process.

### 1. Sample Collection Aspects

In accordance with the disclosed subject matter, and as embodied herein, the methods and systems can include a sample collection to obtain a sample from a subject. For purpose of illustration not limitation, a sample can be obtained from a donor or a patient. The samples obtained by sample collection can then be prepared and amplified for downstream analysis and detection in accordance with the disclosed subject matter as described further herein. For example, but not by way of limitation, sample collection can include any suitable methods and/or techniques for obtaining a sample from a subject.

### 1.1 Sample Types

In certain embodiments, the present disclosure provides methods for obtaining a sample from a subject. In certain embodiments, the sample is a biological sample, e.g., a biological fluid sample. In certain embodiments, the biological fluid sample is a bodily secretion. Non-limiting examples of biological fluid and bodily secretion samples include blood (e.g., whole blood, lysed whole blood, serum, or plasma), saliva or oral fluid, sweat, tears, mucus, urine, lymphatic fluid, cerebrospinal fluid, interstitial fluid, bronchoalveolar lavage fluid or any other sample suitable for analysis using the methods and techniques described herein. In certain embodiments, the biological fluid sample is intended for clinical use, e.g., donor blood for use in transfusion.

In certain embodiments, a sample is derived from blood obtained contemporaneously, e.g., before, during, or after, a subject donates blood. For example, but not by way of limitation, blood can be collected, e.g., into blood collection tubes, contemporaneously with a blood donation being collected into a separate container, e.g., a blood donation collection bag, and the blood contemporaneously collected with the blood donation can provide the source of the samples described herein. Analysis of such samples, as described in detail herein, is thus indicative of whether the contemporaneously collected blood donation contains one or more pathogens or infectious agents. Moreover, release of a blood donation can be predicated, in part or in whole, on analysis of one or more samples sourced from such contemporaneously collected blood.

The methods and systems described herein find use in nucleic acid testing of a sample, e.g., a biological fluid sample. For example, but not by way of limitation, the methods and systems described herein find use in the screening of blood samples generally, irrespective of whether the sample is collected contemporaneously with a blood donation. In instances, however, where the methods and systems described herein are used in connection with the screening of donor blood, such screening can find use in connection with donations of a material, e.g., plasma, platelets, red cells, and whole blood. In certain embodiments, the blood sample screened in the context of the methods and systems described herein is a whole blood sample. In certain embodiments, the blood sample screened in the context of the methods and systems described herein is a lysed whole blood sample. In certain embodiments, the blood sample screened in the context of the methods and systems described herein is a serum sample. In certain embodiments, the blood sample screened in the context of the methods and systems described herein is a plasma sample.

In certain embodiments, the donor biological sample is whole blood. As used herein, “whole blood” refers to blood that has not had any components removed (blood that contains both the fluid and solid components). Transfusion of whole blood, or the red blood cell (RBC) component of whole blood, can increase a patient's oxygen-carrying capacity by effectively increasing the patient's RBC count to thereby increase the amount of available oxygen-carrying hemoglobin. In addition to its oxygen-carrying capacity, whole blood transfusions can be a source of platelets, which aid in blood clotting. In certain embodiments, the clinical use, transfusion of platelets can be used to treat thrombocytopenia, certain cancers, aplastic anemia as well as marrow transplants.

In certain embodiments, the donor biological sample is lysed whole blood. As used herein, “lysed whole blood” refers to blood that has not had any components removed (blood that contains both the fluid and solid components), but where the RBCs have been lysed by exposure to, e.g., a buffer comprising ammonium chloride, potassium carbonate, and EDTA. Ammonium chloride, which lyses RBCs, has minimal effect on lymphocytes. The use of lysed whole blood can be relevant for some NAT screening assays, such as Babesia and Malaria. Babesia and Malaria are parasites that infect and reside within RBCs to evade detection by the host's immune system. Thus, Babesia and Malaria would typically not be present in plasma or serum samples, which lack RBCs, but Babesia and Malaria can be detected in lysed whole blood samples due to the lysis of infected RBCs.

In certain embodiments, the donor biological sample is plasma. Plasma is the aqueous portion of blood that remains after centrifugation to remove the cellular components of blood. Plasma can, in certain embodiments, include albumin, coagulation factors, fibrinolytic proteins, immunoglobulin and other proteins. Products derived from plasma donation can, in certain embodiments, be used to treat bleeding disorders and/or life-threatening trauma/hemorrhages.

In certain embodiments, the donor biological sample is serum. As used herein, “serum” is the clear portion of plasma that does not contain fibrinogen, cells or any solid elements.

The methods and systems described herein can be used for the screening of samples derived from a single individual as well as from a plurality of individuals.

In view of the enhanced efficiencies, and in accordance with the disclosed subject matter, sample pooling may not be necessary for the disclosed systems and methods for time-saving purposes. However, in accordance with one aspect of the disclosed subject matter, sample pooling can be used to the extent desired for incremental time and/or cost savings.

For example, but not by way of limitation, biological samples from a plurality of individuals can be pooled together to generate a pooled sample for NAT-based screening. In certain embodiments, sample pooling will occur in mini-pools or at least two, at least three, at least four, at least five, at least six, at least seven, at least eight, at least nine, at least 10, at least 11, at least 12, at least 13, at least 14, at least 15, at least 16, at least 17, at least 18, at least 19, at least 20, at least 21, at least 22, at least 23, at least 24 samples from individuals, e.g., donors. In certain embodiments, the number of individual samples pooled will be based on factors of eight, e.g., a pool of 8 samples, a pool of 16 samples, a pool of 24 samples, a pool of 32 samples, a pool of 40 samples, a pool of 48 samples, a pool of 56 samples, a pool of 64 samples, a pool of 72 samples, a pool of 80 samples, a pool of 88 samples, a pool of 96 samples, a pool of 104 samples, a pool of 112 samples, a pool of 120 samples, a pool of 128 samples, a pool of 136 samples, a pool of 144 samples, a pool of 152 samples, a pool of 160 samples, a pool of 168 samples, a pool of 176 samples, a pool of 184 samples, a pool of 192 samples, a pool of 200 samples. In certain embodiments, the number of individual samples pooled will be based on factors of two, e.g., a pool of 2 samples, a pool of 4 samples, a pool of 6 samples, a pool of 8 samples, a pool of 10 samples, a pool of 12 samples, a pool of 14 samples, a pool of 16 samples, a pool of 18 samples, a pool of 20 samples, a pool of 22 samples, a pool of 24 samples. In certain embodiments, a pooled sample includes blood from about 2 to about 100 individuals, e.g., from about 5 to about 50, from about 5 to about 20, from about 5 to about 10 or from about 10 to about 20 different individuals. In certain embodiments, e.g., where the pooled sample is a pooled plasma sample, the pooled samples can comprise plasma from 2 to about 10,000 individuals, e.g. from about 2 to about 9,000, from about 2 to about 8,000, from about 2 to about 7,000, from about 2 to about 5,000, from about 2 to about 4,000, from about 2 to about 3,000, from about 2 to about 2,000, from about 2 to about 1,000, from about 2 to about 900, from about 2 to about 800, from about 2 to about 700, from about 2 to about 600, from about 2 to about 500, from about 2 to about 400, from about 2 to about 300, from about 2 to about 200, from about 2 to about 150, from about 2 to about 100, from about 2 to about 50 individuals.

In accordance with an aspect of the disclosed subject matter, samples such as, for example, whole blood lysates or plasma samples can be pooled using onboard hardware (i.e., onboard pooling). For purpose of example, and not limitation, and as embodied herein, the same hardware that manages individual sample preparation can be used to create sample pools of, for example, whole blood and/or plasma. For example, and not limitation, and as embodied herein, individual samples can be introduced to a sample preparation area, such as a sample transport, e.g., a lysis carousel, and two or more of the individual samples can be designated for pooling. For purpose of example and as embodied herein, individual samples in the sample preparation area that have been designated for a pool can then be brought back to the origin of the sample preparation area and pooled together. Processing of the pooled sample can then be performed using the systems and methods described herein for processing non-pooled samples, as described further herein.

Onboard pooling using the same hardware that can manage individual samples can reduce the need for additional laboratory capital, such as separate liquid handlers or poolers. Additionally or alternatively, the use of onboard pooling can reduce or eliminate the need for manual pooling of samples and/or the need for an external liquid handler or pooling system and can improve laboratory workflow. In certain embodiments, the use of sample pooling can increase sample throughput. For example and illustration and not limitation, a system having a throughput of approximately 150 tests per hour for individual samples can incorporate a pool size of 6 samples, which can reduce the number of tests per hour from approximately 150 tests per hour for individual samples to approximately 50 tests per hour of pools of 6 samples. As embodied herein, the reduction in the number of tests per hour can be a result of the sample preparation equipment, such as for example a lysis carousel, being redeployed for pooling samples. For example and not limitation, and as embodied herein, using a pool size of 6 samples, the number of individual samples tested per hour can be as high as approximately 300, which can represent an increase in total throughput of 100%.

As noted above, the samples obtained by the methods of the present disclosure can alternatively be analyzed on an individual-by-individual basis, which can lead to faster processing. In fact, the significant improvements in time to result and overall throughput provided by the methods and systems described herein allow for such individual donor NAT (“ID-NAT”) screening on a scale that is distinct not only “in degree,” but also different “in kind” relative to currently implemented strategies. The methods and systems do not simply facilitate faster donor blood screening, but rather represent a step-change in how such ID-NAT screening occurs, resulting in entirely new strategies for enhancing donor and patient safety and access. For example, the methods and systems described herein allow for both large-scale and small-scale rapid screening of donor blood without the requirement of pooling and/or transportation to a central sample processing facility. Such differences in the operations of donor blood screening facilities provide for improved safety and improved access to blood and blood products. The improvements are particularly evident in resource limited locales as well as in large-scale disasters, e.g., earthquakes and hurricanes, as well as situations where volunteer donation levels drop precipitously, e.g., during the COVID-19 pandemic.

### 1.2 Sampling Aspects

In certain embodiments, a sample, e.g., a biological sample, can be obtained from a subject by any method known in the art. For example, but not by way of limitation, sampling can include obtaining a biological sample from a subject by arterial sampling or venipuncture sampling. Additionally or alternatively, and for example and not limitation, a sample can be obtained using a swab, such as for example, a nasopharyngeal swab.

In certain embodiments, the biological sample is obtained by venipuncture sampling. For example, but not by way of limitation, sampling occurs by inserting a needle through the skin into the lumen of a vein and the filling of one or more blood collection vessel (e.g., blood collection tube, satellite bag, or plasmapheresis bag) with blood from the vein. Typically, sampling occurs by venipuncture sampling where an initial volume of blood is allowed to pass into a diversion bag to capture any skin introduced via the insertion of the needle through the skin, as well as any bacteria that is present on the skin. After a suitable amount of blood has passed into the diversion bag, the blood can be routed to a blood collection vessel for testing, e.g., a blood collection tube, or for storage, e.g., a blood collection bag. In certain embodiments, the blood collection tube will be a sterile glass or plastic test tube comprising a vacuum seal to facilitating the drawing of a predetermined volume of liquid, typically referred to as a “vacutainer.” Sampling can further include labeling the blood collection vessel with information regarding the subject. In certain embodiments, the blood collection vessel includes an anticoagulant, e.g., a powdered anticoagulant or a liquid coagulant, to prevent blood coagulation.

In certain embodiments, the biological sample is centrifuged. For example, but not by way of limitation, a blood sample is centrifuged to separate the plasma from the rest of the sample. In certain embodiments, a blood sample is centrifuged to separate the plasma from the cells of the sample, e.g., erythrocytes, platelets, and/or leukocytes. In certain embodiments, the serum is physically separated by centrifugation from the rest of the sample within about two hours from the time of the collection. In certain embodiments, the RBC component of whole blood can be prepared by automated apheresis methods, which remove blood from a donor, collect the RBCs and return the remaining blood and plasma to the donor. Similarly, plasma can be obtained by automated apheresis methods where blood is removed from a donor, the plasma is collected, and the remaining blood is returned to the donor. In certain embodiments, the sample is obtained by diverting the apheresis line prior to the aphaeretic process to fill one or more blood collection tubes, e.g., vacutainers. Additionally, or alternatively, the sample can be obtained from the collected plasma.

### 2. Sample Preparation Aspects

In accordance with the disclosed subject matter, and as embodied herein, the methods and systems for rapid screening of a sample, e.g., donor blood, include unique sample preparation aspects to isolate nucleic acid from the sample, e.g., the donor blood sample. For example, but not limitation, reference is now made to exemplary sample preparation methods and system components contemplated in the methods and systems of the present disclosure.

As shown in FIG. 1, sample preparation is performed prior to the amplification and detection of the nucleic acids of interest. In certain embodiments, sample preparation as embodied herein includes isolation of the nucleic acids of interest from the sample. The present disclosure also contemplates sample preparation can comprise one or more additional operation(s), e.g., reagent preparation operations, which are performed in conjunction with the sample preparation. In certain embodiments, sample preparation does not include reagent preparation operations.

The sample preparation aspects described herein can involve the use of a variety of suitable sample preparation techniques for the isolation of nucleic acids. For example, but not by limitation, sample preparation can incorporate the use of a variety of sample buffers, nucleic acid immobilization techniques (e.g., immobilization on magnetic particles), and/or elution aspects. Thus, while the methods and systems are described herein with reference to exemplary buffers, immobilization techniques, and elution aspects, one of skill in the art would understand that the methods and systems are not limited to only those exemplary buffers, techniques, and aspects.

As embodied herein, sample preparation methods and system components can be configured to prepare a sample for NAT-based screening. For purpose of illustration but not limitation, the sample preparation methods and components can be configured to isolate and/or purify the nucleic acids in the sample using any suitable sample preparation technique. In certain embodiments, a sample preparation process for use herein includes a lysis process and a wash process to isolate and/or purify the nucleic acids in the sample. In certain embodiments, a wash process can include one or more wash steps and one or more elution steps. In certain embodiments, a sample preparation process for use herein can include a pre-treatment process. In certain embodiments, a sample preparation process for use herein can include an onboard pooling process. In certain embodiments, a sample preparation process for use herein can include an onboard pooling process and a lysis process and a wash process. In certain embodiments, a sample preparation process for use herein can include a pre-treatment process and a lysis process and a wash process. In certain embodiments, a sample preparation process for use herein can include a pre-treatment process and an onboard pooling process and a lysis process and a wash process. In certain embodiments, a sample preparation process can be selected based on the one or more samples to be analyzed. For example and not limitation, when analyzing serum, plasma, and/or lysed whole blood samples, the sample preparation process can include a lysis process and a wash process. Additionally or alternatively, when analyzing serum, plasma, and/or lysed whole blood samples, the sample preparation process can include an onboard pooling process, lysis process and a wash process. As described further herein, incorporating an onboard pooling process in the sample preparation process can, for example, increase throughput (e.g., the number of samples analyzed per unit time). Additionally or alternatively, when analyzing whole blood samples, the sample preparation process can include a pre-treatment process, e.g., a pre-treatment lysis process, lysis process and a wash process. Additionally or alternatively, when analyzing whole blood samples the sample preparation process can include a pre-treatment process, onboard pooling process, lysis process and a wash process. As described further herein, incorporating an onboard pooling process in the sample preparation process can, for example, increase throughput (e.g., the number of samples analyzed per unit time).

In certain embodiments, a sample preparation process for use herein can include mixing, such as for example, mixing of one or more samples and one or more reagents during a pre-treatment process and/or a lysis process. In certain embodiments, a sample preparation process can include mixing during an onboard pooling process. In certain embodiments, a sample preparation process for use herein can include mixing during a pre-treatment process, onboard pooling process, and lysis process. In certain embodiments, the pre-treatment process is a pre-treatment lysis process.

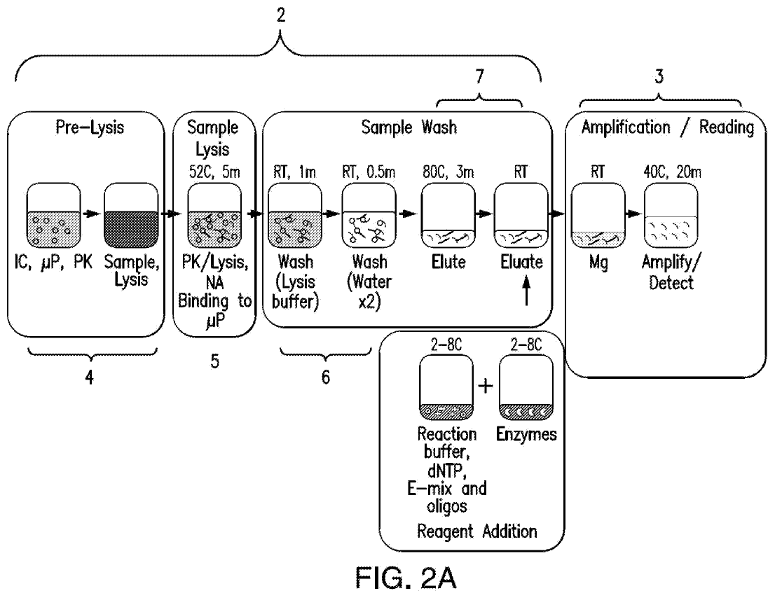

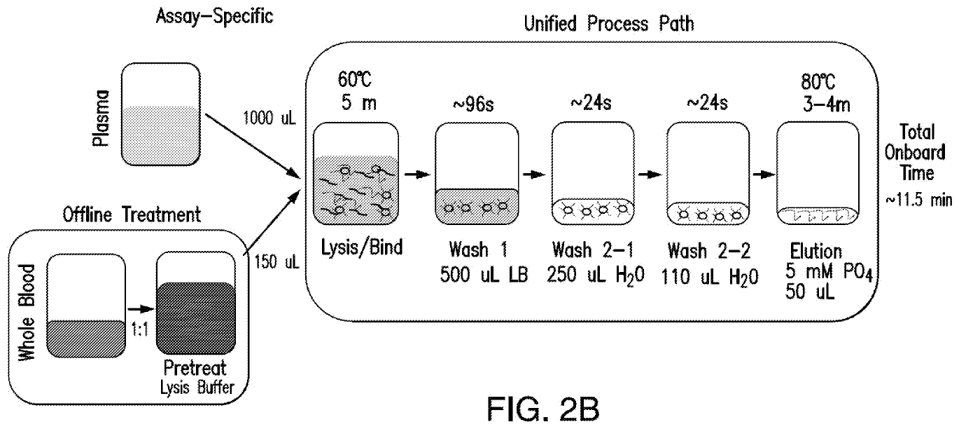

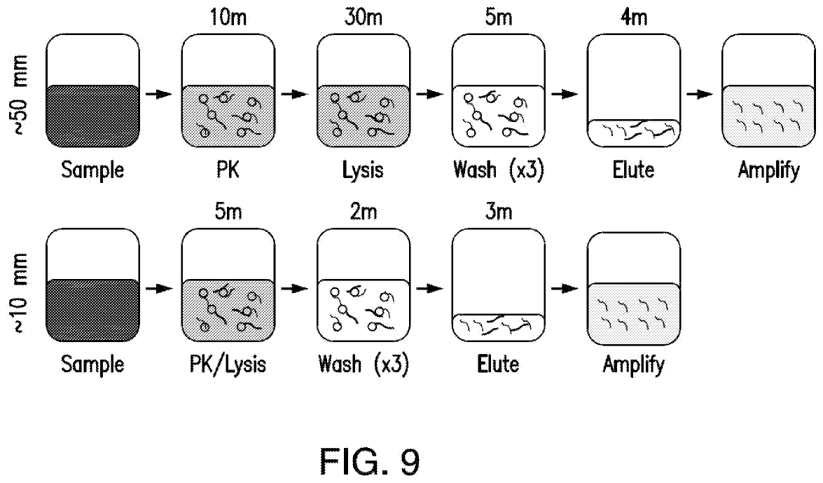

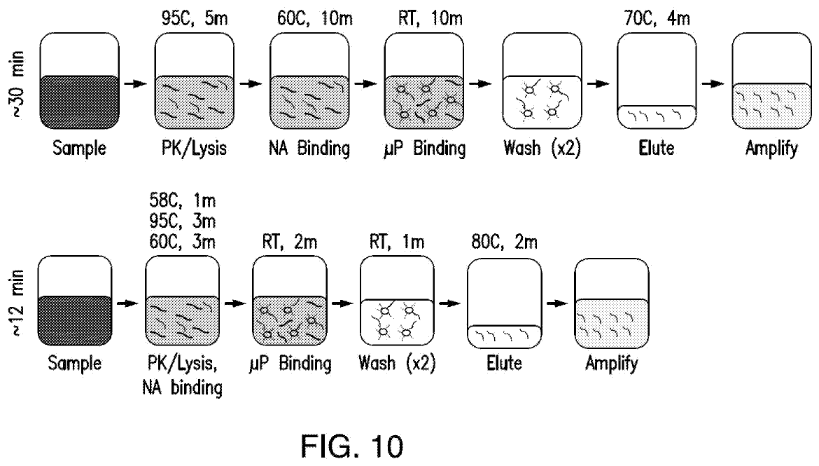

For example, but not limitation, and as illustrated in FIG. 2A, the sample preparation methods and system components embodied herein can be configured to perform a sample preparation process 2 comprising, e.g., the combination 4 of internal control (IC), microparticles (μP) and a protease, e.g., proteinase K (PK), sample, and sample lysis buffer, followed by the incubation 5 of the combined PK/Lysis/sample to promote binding of nucleic acids to the pP. In certain embodiments, the As embodied in FIG. 2A, the rapid sample preparation process 2 can further comprise a wash process 6. In certain embodiments, the wash process 6 can comprise contacting the microparticle bound nucleic acids with a wash fluid, e.g., a lysis buffer or water, for a suitable time and with a suitable number of washes to substantially remove cellular debris and lysis buffer components, e.g., GITC, that can interfere with subsequent amplification and/or detection operations. For example, but not limitation, as embodied in FIG. 2A, the microparticle bound nucleic acids can be washed three times, first with lysis buffer for about 1 minute at room temperature, then twice with water for about 30 seconds each wash (although other suitable durations and temperatures are contemplated as described in detail herein). As embodied in FIG. 2A, the rapid sample preparation process 2 can comprise a rapid elution operation 7. In certain embodiments, the rapid elution operation can comprise contacting the washed microparticle bound nucleic acids with an elution buffer at about 80° C. for about 3 minutes (although other suitable durations and temperatures are contemplated as described in detail herein), after which the microparticles can be separated, e.g., by magnetic transfer of the microparticles or by magnetic retention of the microparticles while the elution buffer comprising eluted nucleic acid is transferred and allowed to cool to prepare the nucleic acid for the amplification and detection process, e.g., amplification and detection process 3. As embodied in FIG. 2A, the amplification and detection process 3 can be initiated by contacting the eluted nucleic acid with a combination of amplification reagents, referred herein as a “MasterMix.” In certain embodiments, the eluted nucleic acid and the “MasterMix” can be contacted with an activator, e.g., a divalent metal ion, e.g., magnesium. In certain embodiments, and as discussed in more detail herein, the amplification reaction can continue for about 20 minutes at 40° C., although other durations, e.g., about 1 minute to about 20 minutes, about 5 minutes to about 20 minutes or about 10 minutes to about 60 minutes and other temperatures, e.g., about 25° C. to about 60° C., can be employed for an amplification and detection process 3. As embodied herein, sample preparation methods and components can perform the sample preparation process. With reference to Examples 5-23, FIGS. 2A-B, 5-7, and 39-41, as embodied herein, the sample preparation process can be completed in about 10 minutes. For example, but not limitation, the sample preparation methods and system components can incorporate a magnetic microparticle-based capture of total nucleic acids, e.g., as illustrated in FIG. 9, or a magnetic microparticle-based direct capture of target nucleic acids, e.g., as illustrated in FIG. 10. For example, but not limitation, as embodied in the magnetic bead-based total nucleic acid capture process depicted in FIG. 9, certain methods and systems described herein can take advantage of one or more sample preparation developments outlined in the lower process of FIG. 9, e.g., combined protease (PK) and sample lysis buffer incubation, rapid wash, and/and rapid elution, to achieve sample preparation in about 10 minutes or less. Individually or in combination, such sample preparation developments significantly reduce overall process times relative to processes employing conventional sample preparation, e.g., as outlined in the top process of FIG. 9. In an alternative example, and as embodied in direct capture-based target nucleic acid capture process depicted in FIG. 10, certain methods and systems described herein can take advantage of one or more rapid sample preparation developments outlined in the lower process of FIG. 10, e.g., combined protease (PK), sample lysis buffer, and nucleic acid binding incubation, rapid microparticle binding, rapid wash, and/and rapid elution, to achieve sample preparation in about 12 minutes. Individually or in combination, such rapid sample preparation developments significantly reduce overall process times relative to processes employing conventional sample preparation, e.g., as outlined in the top process of FIG. 10. Additionally, or alternatively, using the sample preparation developments described herein, e.g., in FIGS. 9 and 10, as embodied herein, a sample preparation process can be completed in about 20 to about 22 minutes for whole blood samples, in about 10 minutes to about 15 minutes for serum/plasma samples, or in about 12 minutes to about 16 minutes, in about 12 minutes to about 14 minutes, in about 10 minutes to about 14 minutes, or in about 10 minutes to about 13 minutes, or in about 10 minutes to about 12 minutes, or even in about 10 minutes to 11 minutes. For purpose of illustration but not limitation, a sample preparation process can include about 3 minutes to about 7 minutes of a lysis process, e.g., about 5 minutes or about 6 minutes. In certain embodiments, a sample preparation process can include about 6 minutes to about 8 minutes of a wash process, e.g., one or more wash steps and/or one or more elution steps. In certain embodiments, a sample preparation process can include about 1 minute to about 3 minutes of one or more sample wash steps, e.g., about 2 minutes to about 3 minutes of one or more sample wash steps. In certain embodiments, sample preparation can include about 2 minutes to about 4 minutes or about 2 minutes to about 5 minutes of one or more elution steps, e.g., about 3 minutes of one or more elution steps. In certain embodiments, a sample preparation process comprises about 4 to about 8 minutes of a lysis process and about 5 to about 9 minutes of a wash process. In certain embodiments, the wash process comprises a first wash step, a second wash step, a third wash step and an elution step.

In certain embodiments, a sample preparation process can start with aspiration of a sample from a sample vessel, e.g., from a sample vessel in a sample tube rack at an aspiration position or from a sample vessel in a sample loading area, and end with dispensing the eluate into a vessel of the amplification and detection system. In certain embodiments, a sample preparation process can be completed in about 12 minutes to about 16 minutes, about 12 minutes to about 15 minutes, about 13 minutes to about 15 minutes or about 13 minutes to about 14 minutes. In certain embodiments, a sample preparation process can be completed in about 12 minutes, about 13 minutes, about 14 minutes, or about 15 minutes. In certain embodiments, a sample preparation process can be completed in about 840 seconds, or about 14 minutes.

As embodied herein, e.g., with reference to FIGS. 2A and 9, the methods and components described herein for sample preparation can employ a sample lysis buffer comprising a protease to reduce overall sample preparation time. Additionally, or alternatively, and as embodied herein, e.g., with reference to FIGS. 2A and 9, the methods and components described herein for rapid sample preparation can employ a sample lysis buffer comprising a protease and microparticles for total nucleic acid capture to reduce overall sample preparation time. Additionally, or alternatively, and as embodied herein, e.g., with reference to FIGS. 2A and 10, the methods and components described herein for rapid sample preparation can employ a sample lysis buffer comprising a protease and microparticles for target nucleic acid capture to reduce overall sample preparation time.

Additionally, or alternatively, and as embodied herein, with reference to FIG. 2B and Examples 5-23, the systems and methods can include a unified process path of lysis, wash, and elution, e.g., for differing sample types. For purpose of illustration but not limitation, the differing sample types (e.g., lysed whole blood, plasma, serum, etc.) can be processed in the same manner along the sample preparation process path, e.g., the sample preparation process path illustrated in FIG. 2B, regardless of the particular nucleic acid analysis subsequently performed. This technique improves efficiency of preparing samples and the flexibility of the overall methods and systems by, among other benefits, providing the capability to modify the specific nucleic acid analysis performed after initiation of sample preparation. As illustrated in FIG. 2B, the unified process path can be initiated with aspiration of a sample from a sample vessel and contacting that sample with sample lysis buffer in the presence of microparticles to allow for binding of nucleic acids to the microparticles. In certain embodiments, the sample aspirated and contacted with lysis buffer will be a plasma sample, a serum sample, or a lysed whole blood sample. In certain embodiments, the plasma, serum, or lysed whole blood sample will have been subjected to an offline treatment prior to aspiration, e.g., whole blood can be centrifuged offline, as described herein, to produce plasma or serum samples, or the whole blood can be treated with a RBC lysis solution, as described below, to produce lysed whole blood samples. In certain embodiments, the sample preparation time for a plasma or a serum sample can be about 15 minutes, e.g., about 14 minutes. In certain embodiments, the sample preparation time for a whole blood sample can be about 22 minutes, e.g., about 20 minutes. The amount of sample aspirated and contacted with sample lysis buffer in the presence of microparticles can vary, e.g., depending on sample type. In certain embodiments, the amount of sample aspirated and contacted with sample lysis buffer in the presence of microparticles can range from about 50 μL to about 2000 μL, and in certain embodiments, e.g., with respect to plasma samples the amount of sample aspirated is about 1000 μL and with respect to lysed whole blood the amount is about 150 μL. In certain embodiments, the sample contacted with sample lysis buffer in the presence of microparticles can be incubated for about 3 minutes to about 7 minutes, e.g., about 5 to about 7 minutes or about 5 to about 6 minutes, at a temperature of about 60° C. In certain embodiments, the unified process path will involve transfer of the nucleic acids bound to microparticles to a first wash buffer. In certain embodiments, the wash buffer can be sample lysis buffer. In certain embodiments, about 500 μL of sample lysis buffer is used as the first wash buffer, although other suitable buffer solutions and volumes are contemplated by the methods and systems described herein. In certain embodiments, the nucleic acids bound to microparticles are washed in the first wash for about 96 seconds, although other suitable wash durations are contemplated by the methods and systems described herein. In certain embodiments, the unified process path will involve transfer of the nucleic acids bound to microparticles to a second wash buffer. In certain embodiments, the second wash buffer can be water. In certain embodiments, about 250 μL of water is used as the second wash buffer, although other suitable buffer solutions and volumes are contemplated by the methods and systems described herein. In certain embodiments, the nucleic acids bound to microparticles are washed in the second wash for about 24 seconds, although other suitable wash durations are contemplated by the methods and systems described herein. In certain embodiments, the unified process path will involve transfer of the nucleic acids bound to microparticles to a third wash employing a third wash buffer, which can, in certain embodiments be water. In certain embodiments, about 110 μL of water is used for the third wash with the third wash buffer, although other suitable buffer solutions and volumes are contemplated by the methods and systems described herein. In certain embodiments, the nucleic acids bound to microparticles are washed in the third wash with the third wash buffer for about 24 seconds, although other suitable wash durations are contemplated by the methods and systems described herein. In certain embodiments, the unified process path will involve transfer of the nucleic acids bound to microparticles to an elution buffer, which can, in certain embodiments comprise 5 mM PO₄. In certain embodiments, about 50 μL of elution buffer is used for the elution, although other suitable buffer solutions and volumes are contemplated by the methods and systems described herein. In certain embodiments, the nucleic acids bound to microparticles are contacted with the elution buffer for about 3 to about 4 minutes, e.g., about 192 seconds, at about 80° C., although other suitable elution durations are contemplated by the methods and systems described herein. Upon removal of the microparticles the eluate prepared by the unified process path can be used in one or more amplification and/or detection processes of a nucleic acid analysis in accordance with the embodiments described herein.

Additionally, or alternatively, and as embodied herein, with reference to FIG. 2B and Examples 5-23, the systems and methods can include sample preparation techniques independently for each sample, thus eliminating the need to employ batch processing. As used herein, batch processing refers to processing a plurality of samples: (1) without the ability to prioritize samples within the group, (2) without the ability to prioritize new samples ahead of those already in process; and/or (3) without the ability to change the nucleic acid analysis associated with any particular sample, e.g., the particular amplification reaction to be performed on the nucleic acids isolated by the sample preparation, after initiation of sample preparation. As described in detail herein, the elimination of batch processing provides significant flexibility to prioritize specific samples and/or specific nucleic acid analyses and enhances overall efficiency of blood surveillance.

Additionally, or alternatively, and as embodied herein, with reference to FIGS. 4 and 9, and Examples 3 and 5-23, the sample preparation systems and methods disclosed herein can capture total sample nucleic acid during the lysis step to facilitate capture of low abundance nucleic acids. For purpose of illustration but not limitation, with reference to Example 3 and FIGS. 2A-2B, the systems and methods can employ microparticles to capture total sample nucleic acid. As embodied herein, the microparticles can include CuTi-coated microparticles. Additionally, or alternatively, microparticles can be combined with the protease and sample lysis buffer to shorten the capture time of total nucleic acids. In certain embodiments, the sample preparation process of the present disclosure can use microparticles, e.g., CuTi-coated microparticles, to capture total sample nucleic acid.

Additionally, or alternatively, with reference to Example 4 below and FIGS. 2A-2B and 10, the systems and methods can directly capture target nucleic acids using capture oligonucleotides immobilized on microparticles to facilitate the isolation of only target nucleic acids. Additionally, or alternatively, microparticles can be combined with the protease and sample lysis buffer to shorten the capture time of target nucleic acids. In certain embodiments, the sample preparation process of the present disclosure can use capture oligonucleotides immobilized on microparticles to capture only target nucleic acids.

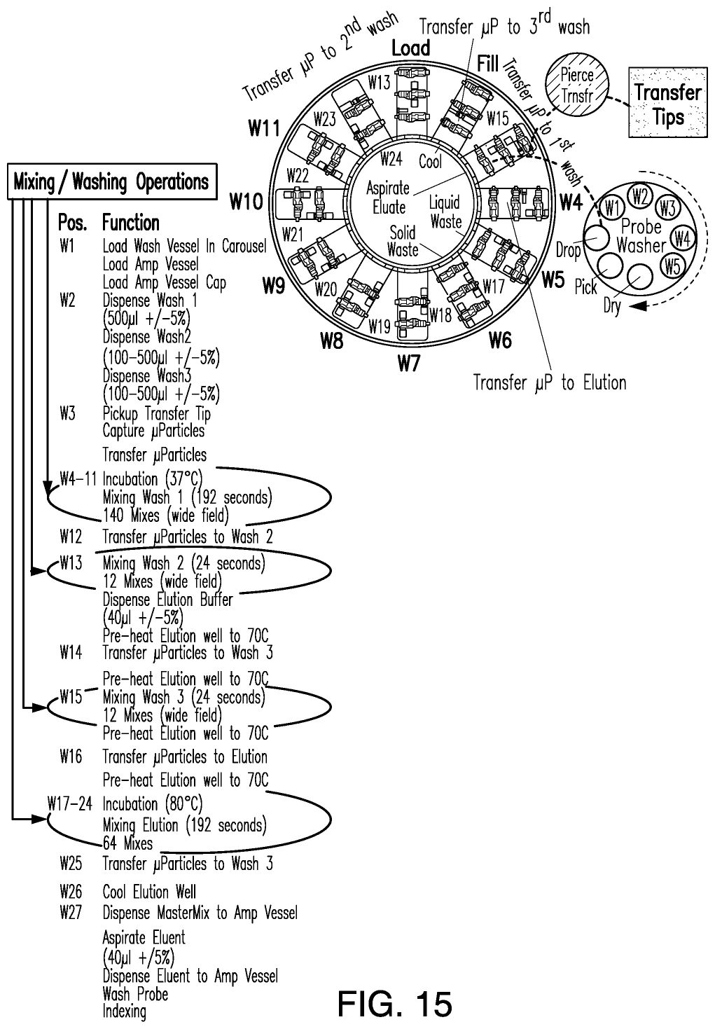

Additionally, or alternatively, as embodied herein, the systems and methods can mix and/or transfer microparticles under magnetic force to increase the mixing and reaction efficiency, e.g., as described in Example 2 and FIGS. 15-17 and 42. For purpose of illustration but not limitation, the type of magnet providing the magnetic force can be, as disclosed herein, an electro-magnet, e.g., a stationary electro-magnet, or a moving permanent magnet. This technique can shorten the capture time of total nucleic acids or target nucleic acids, the wash time of such nucleic acids bound to microparticles, and/or facilitate the transfer to elution solutions in embodiments using microparticles.

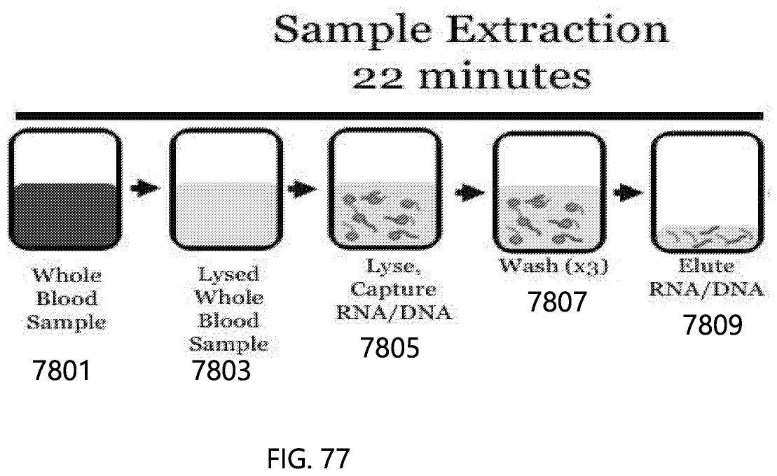

For purpose of example and not limitation, an exemplary sample preparation process for whole blood samples is depicted in FIG. 77. In certain embodiments, a sample preparation process for whole blood samples can include a pre-treatment process. For example, and as embodied herein, at step 7801, a whole blood sample can be added to a vessel. From step 7801 to step 7803, the whole blood sample can be lysed, using for example, a buffer comprising ammonium chloride, potassium carbonate, and EDTA as described further herein. From step 7803 to step 7805, the lysed whole blood can be mixed with reagents, such as for example, a lysis buffer and a protease, microparticles, such as for example CuTi microparticles, and/or an internal control. As described further herein, at step 7805 nucleic acids of interest can bind to the microparticles. As embodied herein, steps 7801 through 7805 can be performed on a sample transport, such as for example, a lysis carousel, as described further herein. From step 7805 to step 7807, the sample can be washed to purify the nucleic acid from the sample. For example, and as embodied herein, the sample can be washed three times. As embodied herein, a first wash can be performed to remove any cellular debris from the sample and/or to remove material that can be weakly bound to the particles. As embodied herein, second and third washes can be performed to remove lysis buffer and/or protease from the sample. As further embodied herein, after washing, at step 7809, the sample can be eluted to capture nucleic acids previously bound to the microparticles. For example, the microparticles can be exposed to an elution buffer, as described further herein. For purpose of example and not limitation, and as embodied herein, wash and elution steps 7807 and 7809 can be performed using a wash vessel and wash track, as described further herein.

### 2.1 Sample Lysis Process

For purpose of illustration and not limitation, the methods and systems of the disclosed subject matter can include a lysis process, i.e., a sample lysis process. In certain embodiments, the sample lysis process includes combining one or more biological samples, e.g., a pooled sample or a non-pooled sample, with a sample lysis buffer. In certain embodiments, the sample lysis buffer is a solution adapted to disrupt the membranes or walls of pathogens, infectious agents, and/or cells present within a sample and release the contents of the pathogens, infectious agents, and/or cells, e.g., nucleic acids present within pathogens, infectious agents, and/or cells.

As described in detail herein, introduction of sample lysis buffer into a sample facilitates the release of nucleic acids from the pathogens, infectious agents, and/or the cells of the sample. The introduction of such sample lysis buffer is distinguished from the preparation of the “sample type” referred herein as “lysed whole blood.” As noted herein, lysed whole blood is a sample type of the present disclosure where the RBCs have been lysed by exposure to a lysis buffer, e.g., an RBC lysis solution (e.g., a buffer comprising ammonium chloride, potassium carbonate, and EDTA) during a pre-treatment lysis process. While such ammonium chloride-containing buffers lyse RBCs, such buffers have minimal effect on lymphocytes and therefore would not fall within the scope of a “sample lysis buffer” as used herein.

In certain embodiments, e.g., as illustrated in FIG. 2A and the bottom process path of FIG. 9, the methods and systems of the disclosed subject matter can achieve improved time to result and improved throughput per unit size, among other benefits, due, at least in part, to the use of particular sample lysis buffers to perform a rapid sample preparation. In certain embodiments, a sample preparation process is accomplished by contacting the sample with a sample lysis buffer comprising both: (1) a solution adapted to disrupt the membranes or walls of pathogens, infectious agents, and/or cells present within a sample and release the contents of the pathogens, infectious agents, and/or cells, e.g., a conventional sample lysis buffer; and (2) a protease, e.g., Proteinase K, which would conventionally be used in connection with a separate, pre-lysis solution to inactivate nucleases (enzymes that would degrade the nucleic acid released during exposure of a sample to a sample lysis buffer) and degrade proteins covalently or non-covalently bound to nucleic acids, e.g., proteins covalently bound to HBV DNA. In certain embodiments, the additional of protease, e.g., Proteinase K, to the sample is optional. For example, but not by way of limitation, a sample preparation process can be accomplished by contacting the sample with a sample lysis buffer (e.g., in the absence of a protease, e.g., Proteinase K).

Additionally, or alternatively, e.g., as illustrated in the top process path of FIG. 9, the sample lysis process employs a conventional sample lysis buffer, e.g., a buffer that does not comprise a protease. For example, such sample preparation methods and components can employ, prior to contacting the sample with the conventional sample lysis buffer, a pre-conditioning of the sample by contacting the sample with a pre-conditioning solution containing a protease to inactivate nucleases and degrade proteins covalently or non-covalently bound to nucleic acids, e.g., proteins covalently bound to HBV DNA.

Additionally, or alternatively, e.g., the methods and systems illustrated in FIGS. 2-3 and 5-6, the present disclosure is directed to methods and systems configured to initiate contact of a sample with a sample lysis buffer in what are referred to as “Pre-Lysis systems.” In certain embodiments, the systems that are configured to then incubate the sample in the presence of a sample lysis buffer are referred herein as “Lysis systems.” Thus, as used herein, Pre-Lysis systems generally combine the sample from a donor (or pool of donors) with sample lysis buffer and microparticle-containing reagents, which then undergo lysis and nucleic acid capture in the Sample Lysis system. The use of “Pre-Lysis” in the context of these systems thus differs from the use of “pre-conditioning” in the context of solutions comprising proteases for the purpose of inactivating nucleases and degrade proteins covalently or non-covalently bound to nucleic acids.

Additionally, or alternatively, a sample lysis buffer for use in the present disclosure can include one or more of the following components: a protease, a detergent, a protein denaturant, and a buffer. In certain embodiments, a sample lysis buffer for use in the present disclosure includes a protease, a detergent, a buffer, and a protein denaturant. In certain embodiments, the protease is optional.

Additionally, or alternatively, the protease is an enzyme that will degrade or otherwise inactivate one or more nuclease and/or degrade proteins covalently or non-covalently bound to nucleic acids. In certain embodiments, the protease is a serine protease. In certain embodiments, the protease is Proteinase K.

In certain embodiments, the detergent can be nonionic, anionic and/or zwitterionic. Non-limiting examples of detergents include Triton-X, e.g., Triton X-100 (2-[4-(2,4,4-trimethylpentan-2-yl)phenoxy]ethanol) or Triton X-114 (2-[2-[4-(2,4,4-trimethylpentan-2-yl)phenoxy]ethoxy]ethanol), Tween (Polyoxyethylene sorbitan monolaurate), e.g., Tween-20 (Polyoxyethylene (20) sorbitan monolaurate) or Tween-80 (Polyoxyethylene (80) sorbitan monolaurate), sodium dodecyl sulfate (SDS), octyl thioglucoside, octyl glucoside and 3-[(3-cholamidopropyl)dimethylammonio]-1-propanesulfonate (CHAPS). In certain embodiments, the detergent is a nonionic detergent, e.g., Tween.

In certain embodiments, the sample lysis buffer has a pH from about 5.5 to about 8.0, e.g., a pH of about 6.0, about 6.5, about 7.0 or about 7.5. In certain embodiments, the pH of the sample lysis buffer depends on the sample to be lysed. For example, but not by way of limitation, the pH of the sample lysis buffer can be 6.0. In certain embodiments, the pH of the sample lysis buffer can be about 7.8.

In certain embodiments, the sample lysis buffer comprises a chaotropic agent, a buffer and a detergent. Non-limiting examples of chaotropic agents include guanidinium salts (e.g., guanidinium thiocyanate, guanidinium hydrochloride, guanidinium chloride and guanidinium isothiocyanate), urea, potassium iodide, perchlorates (e.g., potassium perchlorate) and other types of thiocyanates. In certain embodiments, the chaotropic agent is guanidinium thiocyanate (GITC). In certain embodiments, the lysis buffer can further include a protease.

In certain embodiments, the sample lysis buffer comprises about 2.5 to about 4.7 M GITC and about 2% to about 10% Tween-20. In certain embodiments, e.g., the sample lysis buffer for plasma or serum samples comprises about 4.7 M GITC, about 10% Tween-20, and a pH of about 7.8. In certain embodiments, e.g., the sample lysis buffer for whole blood samples comprises about 3.5 M GITC, about 2.5% Tween-20, and a pH of about 6.0. In certain embodiments, the sample lysis buffer comprises 3.13 M GITC, 6.7% Tween-20, 100 mM Tris, and a pH of about 7.8.

Additionally, or alternatively, the volume of sample lysis buffer added to the sample depends on the volume of the sample. In certain embodiments, about 10 μl to about 1000 μl, e.g., about 100 μl to about 1000 μl, of sample lysis buffer can be added to a sample. In certain embodiments, about 750 μl of lysis buffer can be added to the sample. In certain embodiments, the ratio of the volume of sample lysis buffer to the volume of sample is from about 1:100 to about 100:1. In certain embodiments, the ratio of the volume of sample lysis buffer to the volume of sample is about 1:1. In certain embodiments, the ratio of the volume of sample lysis buffer to the volume of sample is about 0.75:1.

Additionally, or alternatively, the sample can be incubated for a sufficient amount of time to promote lysis. For example, but not by way of limitation, the total incubation time for sample lysis can be from about 60 seconds to about 6,000 seconds. In certain embodiments, the total incubation time for sample lysis can be from about 60 to about 5,000 seconds. In certain embodiments, the total incubation time for sample lysis can be from about 60 to about 4,000 seconds. In certain embodiments, the total incubation time for sample lysis can be from about 60 to about 3,000 seconds. In certain embodiments, the total incubation time for sample lysis can be from about 60 to about 2,000 seconds. In certain embodiments, the total incubation time for sample lysis can be from about 60 to about 1,000 seconds. In certain embodiments, the total incubation time for sample lysis can be from about 60 to about 600 seconds. In certain embodiments, the total incubation time for sample lysis can be from about 60 to about 540 seconds. In certain embodiments, the total incubation time for sample lysis can be from about 60 to about 480 seconds. In certain embodiments, the total incubation time for sample lysis can be from about 60 to about 420 seconds. In certain embodiments, the total incubation time for sample lysis can be from about 60 to about 360 seconds. In certain embodiments, the total incubation time for a sample can be from about 60 to about 300 seconds. In certain embodiments, the total incubation time for sample lysis can be from about 60 to about 240 seconds. In certain embodiments, the total incubation time for sample lysis can be from about 60 to about 180 seconds. In certain embodiments, the total incubation time for sample lysis can be from about 60 to about 120 seconds. In certain embodiments, the total incubation time for sample lysis can be from about 180 to about 6,000 seconds. In certain embodiments, the total incubation time for sample lysis can be from about 180 to about 5,000 seconds. In certain embodiments, the total incubation time for sample lysis can be from about 180 to about 4,000 seconds. In certain embodiments, the total incubation time for sample lysis can be from about 180 to about 3,000 seconds. In certain embodiments, the total incubation time for sample lysis can be from about 180 to about 2,000 seconds. In certain embodiments, the total incubation time for sample lysis can be from about 180 to about 1,000 seconds. In certain embodiments, the total incubation time for sample lysis can be from about 180 to about 600 seconds. In certain embodiments, the total incubation time for sample lysis can be from about 180 to about 540 seconds. In certain embodiments, the total incubation time for sample lysis can be from about 180 to about 480 seconds. In certain embodiments, the total incubation time for sample lysis can be from about 180 to about 420 seconds. In certain embodiments, the total incubation time for sample lysis can be from about 180 to about 360 seconds. In certain embodiments, the total incubation time for sample lysis can be from about 180 to about 300 seconds. In certain embodiments, the total incubation time for sample lysis can be from about 180 to about 240 seconds. In certain embodiments, the total incubation time for sample lysis can be from about 240 to about 6,000 seconds. In certain embodiments, the total incubation time for sample lysis can be from about 240 to about 5,000 seconds. In certain embodiments, the total incubation time for sample lysis can be from about 240 to about 4,000 seconds. In certain embodiments, the total incubation time for sample lysis can be from about 240 to about 3,000 seconds. In certain embodiments, the total incubation time for sample lysis can be from about 240 to about 2,000 seconds. In certain embodiments, the total incubation time for sample lysis can be from about 240 to about 1,000 seconds. In certain embodiments, the total incubation time for sample lysis can be from about 240 to about 600 seconds. In certain embodiments, the total incubation time for sample lysis can be from about 240 to about 540 seconds. In certain embodiments, the total incubation time for sample lysis can be from about 240 to about 480 seconds. In certain embodiments, the total incubation time for sample lysis can be from about 240 to about 420 seconds. In certain embodiments, the total incubation time for sample lysis can be from about 240 to about 360 seconds. In certain embodiments, the total incubation time for sample lysis can be from about 240 to about 300 seconds. In certain embodiments, the total incubation time for sample lysis can be from about 300 to about 6,000 seconds. In certain embodiments, the total incubation time for sample lysis can be from about 300 to about 5,000 seconds. In certain embodiments, the total incubation time for sample lysis can be from about 300 to about 4,000 seconds. In certain embodiments, the total incubation time for sample lysis can be from about 300 to about 3,000 seconds. In certain embodiments, the total incubation time for sample lysis can be from about 300 to about 2,000 seconds. In certain embodiments, the total incubation time for sample lysis can be from about 300 to about 1,000 seconds. In certain embodiments, the total incubation time for sample lysis can be from about 300 to about 600 seconds. In certain embodiments, the total incubation time for sample lysis can be from about 300 to about 540 seconds. In certain embodiments, the total incubation time for sample lysis can be from about 300 to about 480 seconds. In certain embodiments, the total incubation time for sample lysis can be from about 300 to about 420 seconds. In certain embodiments, the total incubation time for sample lysis can be from about 300 to about 360 seconds. In certain embodiments, the total incubation time for sample lysis can be from about 360 to about 6,000 seconds. In certain embodiments, the total incubation time for sample lysis can be from about 360 to about 5,000 seconds. In certain embodiments, the total incubation time for sample lysis can be from about 360 to about 4,000 seconds. In certain embodiments, the total incubation time for sample lysis can be from about 360 to about 3,000 seconds. In certain embodiments, the total incubation time for sample lysis can be from about 360 to about 2,000 seconds. In certain embodiments, the total incubation time for sample lysis can be from about 360 to about 1,000 seconds. In certain embodiments, the total incubation time for sample lysis can be from about 360 to about 600 seconds. In certain embodiments, the total incubation time for sample lysis can be from about 360 to about 540 seconds. In certain embodiments, the total incubation time for sample lysis can be from about 360 to about 480 seconds. In certain embodiments, the total incubation time for sample lysis can be from about 360 to about 420 seconds. In certain embodiments, the total incubation time for sample lysis can be from about 360 to about 400 seconds. In certain embodiments, the total incubation time for sample lysis can be from about 420 to about 6,000 seconds. In certain embodiments, the total incubation time for sample lysis can be from about 420 to about 5,000 seconds. In certain embodiments, the total incubation time for sample lysis can be from about 420 to about 4,000 seconds. In certain embodiments, the total incubation time for sample lysis can be from about 420 to about 3,000 seconds. In certain embodiments, the total incubation time for sample lysis can be from about 420 to about 2,000 seconds. In certain embodiments, the total incubation time for sample lysis can be from about 420 to about 1,000 seconds. In certain embodiments, the total incubation time for sample lysis can be from about 420 to about 600 seconds. In certain embodiments, the total incubation time for sample lysis can be from about 420 to about 540 seconds. In certain embodiments, the total incubation time for sample lysis can be from about 420 to about 480 seconds. In certain embodiments, the total incubation time for sample lysis can be from about 480 to about 6,000 seconds. In certain embodiments, the total incubation time for sample lysis can be from about 480 to about 5,000 seconds. In certain embodiments, the total incubation time for sample lysis can be from about 480 to about 4,000 seconds. In certain embodiments, the total incubation time for sample lysis can be from about 480 to about 3,000 seconds. In certain embodiments, the total incubation time for sample lysis can be from about 480 to about 2,000 seconds. In certain embodiments, the total incubation time for sample lysis can be from about 480 to about 1,000 seconds. In certain embodiments, the total incubation time for sample lysis can be from about 480 to about 600 seconds. In certain embodiments, the total incubation time for sample lysis can be from about 480 to about 540 seconds. In certain embodiments, the total incubation time for sample lysis can be from about 540 to about 6,000 seconds. In certain embodiments, the total incubation time for sample lysis can be from about 540 to about 5,000 seconds. In certain embodiments, the total incubation time for sample lysis can be from about 540 to about 4,000 seconds. In certain embodiments, the total incubation time for sample lysis can be from about 540 to about 3,000 seconds. In certain embodiments, the total incubation time for sample lysis can be from about 540 to about 2,000 seconds. In certain embodiments, the total incubation time for sample lysis can be from about 540 to about 1,000 seconds. In certain embodiments, the total incubation time for sample lysis can be from about 540 to about 600 seconds. In certain embodiments, the total incubation time for sample lysis can be from about 600 to about 6,000 seconds. In certain embodiments, the total incubation time for sample lysis can be from about 600 to about 5,000 seconds. In certain embodiments, the total incubation time for sample lysis can be from about 600 to about 4,000 seconds. In certain embodiments, the total incubation time for sample lysis can be from about 600 to about 3,000 seconds. In certain embodiments, the total incubation time for sample lysis can be from about 600 to about 2,000 seconds. In certain embodiments, the total incubation time for sample lysis can be from about 600 to about 1,000 seconds. In certain embodiments, the total incubation time for sample lysis can be about 384 seconds. In certain embodiments, the total incubation time for sample lysis can be at least about 60 seconds, at least about 120 seconds, at least about 180 seconds, at least about 240 seconds, at least about 300 seconds, at least about 360 seconds, at least about 420 seconds, at least about 480 seconds, at least about 540 seconds or at least about 600 seconds.

In certain embodiments, the sample can be incubated at a temperature from about 37° C. to about 60° C., e.g., from about 50° C. to about 60° C. For example, but not by way of limitation, the temperature can be about 50° C., about 51° C., about 52° C., about 53° C., about 54° C., about 55° C., about 56° C., about 57° C., about 58° C., about 58° C. or about 60° C. In certain embodiments, the sample can be incubated at a temperature from about 50° C. to about 60° C.

Additionally, or alternatively, and particularly with reference to FIG. 2B and Examples 5-23, the systems and methods can include a unified process path of lysis, wash, and elution. For purpose of illustration but not limitation, the same sample type (e.g., whole blood, plasma, serum, etc.) can be processed in the same manner along the sample preparation process path, e.g., the sample preparation process path illustrated in FIG. 2B, regardless of the particular nucleic acid analysis subsequently performed. Thus, with the initiation of the sample lysis process, e.g., by aspiration of the sample from a sample vessel, and concluding with the elution of nucleic acid to generate a eluate for subsequent amplification and detection as disclosed herein or concluding with the transfer of the eluate to a vessel of the amplification and detection system as disclosed herein, this unified process path technique can improve the efficiency of preparing samples and the flexibility of the overall methods and systems by, among other benefits, providing the capability to modify the specific nucleic acid analysis performed after initiation of sample preparation.

### 2.2 Wash & Elution Steps

In accordance with another aspect of the disclosed subject matter, the methods and systems herein can include wash process that includes one or more wash and elution steps. In certain embodiments, the wash process begins by the washing of microparticles in a first wash step and ends with the transfer of an eluate to a vessel of the amplification and detection system. In certain embodiments, the wash process is part of the sample preparation process. For example, but not by way of limitation, the wash process follows the lysis process in the sample preparation process.

As described herein in more detail below, the wash and elution steps (e.g., each of the wash process) of the present disclosure provide distinct advantages that, at least in part, support the significant improvements in time to result and improved throughput per unit size, among other benefits, that are achieved by the methods and systems of the present disclosure. For example, the wash and elution steps can be configured to elute nucleic acid that can be analyzed by any of the various nucleic acid analyses described herein to detect a pathogen or infectious agent, allowing for changes to the pathogen or infectious agent to be detected, even after the sample has been prepped for amplification and detection.

In certain embodiments, the at least one wash step generates at least one wash and the at least one elution generates at least one eluate. In certain embodiments, the wash and elute step is performed after the lysis step as shown in FIGS. 2A-2B and FIG. 79. The wash and elute steps are generally employed to purify the nucleic acids from the sample, as well as to remove any cellular debris and/or lysis buffer components, e.g., GITC, that can inhibit the amplification and/or detection operations.

A variety of suitable techniques or methods for purifying nucleic acids from sample can be used in connection with the wash and/or elution steps. For example, but not by way of limitation, the nucleic acids present within a sample can be isolated by the use of microparticles, e.g., CuTi microparticles, that can bind nucleic acids, including the nucleic acids of interest. In certain embodiments, direct nucleic acid capture, e.g., the use of microparticles (for example, magnetic glass particles), or other solid supports coated with nucleic acids complementary to target nucleic acids, can be used, e.g., as shown in FIG. 4. For purpose of illustration not limitation, the wash and elution steps can be performed on a wash track system with a plurality of positions loaded with wash vessels.

In certain embodiments, the nucleic acids present with a sample can be isolated by contacting the sample with a lysis buffer, microparticles, e.g., CuTi microparticles, and, optionally, a protease, e.g., Proteinase K. In certain embodiments, the nucleic acids present with a sample can be isolated by contacting the sample with a lysis buffer, internal control (IC) nucleic acids, microparticles, e.g., CuTi microparticles, and, optionally, a protease, e.g., Proteinase K.

### 2.3 Microparticle-Based Total Nucleic Acid Capture

Further in accordance with the disclosed subject matter, the significant improvements in time and efficiency without sacrifice to sensitivity to result in improved throughput per unit size, among other benefits, are achieved by the methods and systems of the present disclosure are due, at least in part, to the use of microparticle-based total nucleic acid capture. Microparticle-based total nucleic acid capture refers, in certain embodiments, to the use of microparticles capable of non-selective binding to nucleic acids, thus allowing for the capture of nucleic acids irrespective of sequence. As disclosed herein, having bound nucleic acids, the microparticles can be washed to remove non-nucleic acid components of the sample during one or more wash steps of a wash process. The washed microparticles can then be exposed to conditions that cause the elution of the bound nucleic acid. Because microparticle-based total nucleic acid capture is not dependent on a sequence-specific interaction, it can facilitate the capture of low abundance nucleic acids and/or nucleic acids having similar sequences that might compete for binding in a sequence-specific approach. One type of microparticle that can be used in the context of microparticle-based total nucleic acid capture are copper titanium (“CuTi”) microparticles. As disclosed herein, the sample lysis, wash, and elute steps employ a microparticle-based total nucleic acid capture. An exemplary CuTi microparticle-based total nucleic acid capture aspect for use in the present disclosure is provided in U.S. Patent Publication No. 2017/0081655, the contents of which are incorporated herein in its entirety.

As shown in FIG. 4, an exemplary microparticle-base total nucleic acid capture aspect includes the use of CuTi microparticles for binding nucleic acids within a sample, e.g., a lysed sample. CuTi microparticles, in particular, can allow for expedited purification of nucleic acids relative to conventional aspects comprising multiple organic extraction procedures. Additional benefits of the use of CuTi microparticles are described in detail in U.S. Pat. No. 10,526,596, which is incorporated herein in its entirety.

Additionally, or alternatively, the microparticles used in connection with the methods of the present disclosure, e.g., CuTi microparticles, have a diameter of about 0.5 to about 50 μm, e.g., about 0.5 μm, about 1.0 μm, about 1.5 μm, about 2.0 μm, about 5.0 μm, about 10.0 μm, about 20.0 μm, about 30.0 μm, about 40.0 μm or about 50.0 μm. In certain embodiments, the CuTi is present in the CuTi microparticles at a ratio of about 2:1 Cu to Ti, e.g., about 3 to about 1, about 2 to about 1, about 1 to about 1, about 1 to about 2 or about 1 to about 3.

In certain embodiments, the microparticles can be used in the methods of the present disclosure at a particle volume from about 1 μl to about 100 μl. For example, but not by way of limitation, the microparticles can be used in the methods of the present disclosure at a particle volume from about 5 μl to about 100 μl, about 5 μl to about 50 μl or about 10 μl to about 50 μl.

Additionally, or alternatively, the microparticles used in connection with the methods of the present disclosure, e.g., CuTi microparticles, can bind at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99% or at least about 100% of the nucleic acids, e.g., DNA or RNA, in the sample.

Additionally, or alternatively, subsequent to binding of nucleic acids to the microparticles used in connection with the methods of the present disclosure, e.g., CuTi microparticles, the microparticles are washed using a wash process as disclosed hereinto remove contaminants and/or undesired material from the microparticles in the sample. In certain embodiments, the wash process, e.g., wash and elute steps, can include more than one wash. For example, but not by way of limitation, the wash process includes at least about 2 washes, at least 3 washes or at least 4 washes. In certain embodiments, the wash process includes at least about 3 washes. In certain embodiments, the wash process includes 3 washes, as shown in Example 8, e.g., for use in capture of total nucleic acids. In certain embodiments, the wash process includes 2 washes, as shown in Example 4, e.g., for use in capture of target nucleic acids.

In certain embodiments, the microparticles used in connection with the methods of the present disclosure, e.g., CuTi microparticles, are washed in each wash solution for about 10 seconds to about 5 minutes, e.g., from about 20 seconds to about 2 minutes, from about 20 seconds to about 1 minute, from about 20 seconds to about 48 seconds, from about 20 second to about 30 seconds, from about 30 second to about 90 seconds or from about 30 second to about 50 seconds. In certain embodiments, the microparticles are washed in each wash solution for about 40 seconds. In certain embodiments, at least one of the washes includes a detergent and/or a protein denaturant. Non-limiting examples of detergents are described herein.

Additionally, or alternatively, the microparticles used in connection with the methods of the present disclosure, e.g., CuTi microparticles, can be removed from the sample and placed in the wash solution. For example, but not by way of limitation, the microparticles used in connection with the methods of the present disclosure, e.g., CuTi microparticles, can be captured using magnets and transferred to a well containing a wash solution. For example and not limitation, microparticles can be transferred using a magnetic tip. Additionally or alternatively, in certain embodiments microparticles can be transferred with a plunger. Additionally or alternatively, microparticles can be transferred with a moving magnet or with a stationary magnet. In certain embodiments, the microparticles used in connection with the methods of the present disclosure, e.g., CuTi microparticles, can be captured and placed into another wash solution.

Additionally, or alternatively, a wash solution for use in the present disclosure has a pH from about 5.5 to about 8.0, e.g., a pH of about 6.0, about 6.5, about 7.0 or about 7.5. In certain embodiments, the pH of a wash solution depends on the sample. For example, but not by way of limitation, the pH of a wash buffer can be 6.0. In certain embodiments, the pH of the wash solution can be about 7.8.

Additionally, or alternatively, a volume of about 10 μl to about 500 μl of wash solution can be used for each wash. For example, but not by way of limitation, a volume of about 100 μl to about 500 μl of wash solution, e.g., about 250 μl, can be used for each wash.

In certain embodiments, at least one of the washes is performed using a lysis buffer. In certain embodiments, one of the washes is performed using a wash solution, e.g., a first wash solution, comprising about 2.5 M to about 4.7 M GITC and about 2% to about 10% Tween-20, and a pH of about 5.5 to about 8.0. In certain embodiments, at least one of the washes is performed using a wash solution, e.g., a first wash solution for washing a plasma or serum sample, comprising about 4.7 M GITC, about 10% Tween-20, and a pH of about 7.8. In certain embodiments, at least one of the washes is performed using a wash solution, e.g., a first wash solution for washing a whole blood sample, comprising about 3.5 M GITC, about 2.5% Tween-20, and a pH of about 6.0. In certain embodiments, at least one of the washes is performed using a wash solution, e.g., a first wash solution for washing a whole blood sample, comprising about 3.13 M GITC, 6.7% Tween-20, 100 mM Tris, and a pH of about 7.8. In certain embodiments, at least one of the washes is performed with water. For example, but not by way of limitation, the first, second and third wash can be performed with water. In certain embodiments, the second and third wash can be performed with water. In certain embodiments, the first wash is performed with a lysis buffer. In certain embodiments, the first wash is performed with a lysis buffer and the second and third washes are performed with water.

Additionally, or alternatively, the bound nucleic acids are subsequently eluted from the microparticles used in connection with the methods of the present disclosure, e.g., CuTi microparticles, after washing. For example, but not by way of limitation, the microparticles used in connection with the methods of the present disclosure, e.g., CuTi microparticles, are captured from the final wash solution, e.g., third wash solution, and placed in an elution buffer. In certain embodiments, the elution of the nucleic acids from the microparticles used in connection with the methods of the present disclosure, e.g., CuTi microparticles, using an elution buffer generates an eluate comprising the previously bound nucleic acids. In certain embodiments, the elution buffer comprises about 5 mM to about 10 mM phosphate and has a pH of about 7.5 to about 9.0. In certain embodiments, the elution buffer comprises a 5 mM PO₄ solution. In certain embodiments, the elution step comprises incubating the microparticles that have bound nucleic acids in a 5 mM PO₄ solution at 80° C., e.g., for about 3 minutes.

Additionally, or alternatively, the microparticles used in connection with the methods of the present disclosure, e.g., CuTi microparticles, are incubated in the elution buffer for about 1 minute to about 10 minutes, e.g., for about 2 minutes to about 9 minutes, for about 3 minutes to about 8 minutes, from about for about 2 minutes to about 4 minutes or from about for about 3 minutes to about 4 minutes. In certain embodiments, the microparticles used in connection with the methods of the present disclosure, e.g., CuTi microparticles, are incubated in the elution buffer for about 3 minutes. In certain embodiments, the microparticles used in connection with the methods of the present disclosure are incubated in the elution buffer for about 200 second or less, e.g., about 192 seconds or less. In certain embodiments, the microparticles used in connection with the methods of the present disclosure, e.g., CuTi microparticles, are incubated in the elution buffer at a temperature from about 60° C. to about 100° C., e.g., from about 70° C. to about 90° C. or from about 75° C. to about 85° C. In certain embodiments, the microparticles used in connection with the methods of the present disclosure, e.g., CuTi microparticles, are incubated in the elution buffer at a temperature of about 80° C. The resultant eluate can then be used for a subsequent amplification reaction and is, in certain embodiments, capable of being used in any of the various nucleic acid analyses described herein to detect a pathogen or infectious agent. In certain embodiments, the resulting eluate has a volume of about 5 μl to about 500 μl, e.g., about 10 μl to about 250 μl or about 10 μl to about 100 μl.

While the above-described exemplary embodiments reference the use of CuTi microparticles, the methods and systems of the present disclosure can incorporate, in addition to CuTi microparticles or as alternatives to CuTi microparticles, a wide variety of particles and/or solid supports to facilitate the isolation of nucleic acids, e.g., target nucleic acids. For example, but not by way of limitation, the methods and systems of the present disclosure can utilize particles, e.g., microparticles, and/or solid supports comprising or coated with a wide variety of metal oxides (See, e.g., U.S. Pat. No. 6,936,414; herein incorporated by reference in its entirety). The present disclosure is not, however, limited to particular metal oxides. In certain embodiments, the metal or metal oxide is AlTi, CaTi, CoTi, Fe₂Ti, Fe₃Ti, MgTi, MnTi, NiTi, SnTi, ZnTi, Fe₂O₃, Fe₃O₄, Mg, Mn, Sn, Ti, or Zn (e.g., an hydrated or hydrated forms). Moreover, in certain embodiments, the particles and/or solid surfaces are comprised of organic polymers such as polystyrene and derivatives thereof, polyacrylates and polymethacrylates, and derivatives thereof or polyurethanes, nylon, polyethylene, polypropylene, polybutylene, and copolymers of these materials. In certain embodiments, particles are polysaccharides, in particular hydrogels such as agarose, cellulose, dextran, Sephadex, Sephacryl, chitosan, inorganic materials such as, e.g., glass or further metal oxides and metalloid oxides (e.g., oxides of formula MeO, wherein Me is selected from, e.g., Al, Ti, Zr, Si, B, in particular Al₂O₃, TiO₂, silica and boron oxide) or metal surfaces, e.g., gold.

Additionally, or alternatively, particles are magnetic (e.g., paramagnetic, ferrimagnetic, ferromagnetic or superparamagnetic), but the methods and systems of the present disclosure are non-magnetic. In some embodiments, the particles and/or solid surface can have a planer, acicular, cuboidal, tubular, fibrous, columnar or amorphous shape, although other geometries are specifically contemplated.

### 2.4 Microparticle-Based Direct Capture of Target Nucleic Acid

As further disclosed herein, significant improvements in time and efficiency without sacrifice of sensitivity can improve throughput per unit size, among other benefits, by the methods and systems of the present disclosure are based, at least in part, upon the use of microparticle-based direct capture of target nucleic acids in the sample lysis, wash, and elution steps. As illustrated in FIG. 4, microparticle-based direct capture of target nucleic acids can include the use of capture oligonucleotides, i.e., oligonucleotides complementary to the target nucleic acids of interest, bound to microparticles to capture the target nucleic acids of interest in a sequence-selective manner. The direct capture of target nucleic acids thus differs from other systems that capture total nucleic acid, which are not sequence selective. By limiting the nucleic acids captured and thus ultimately eluted to the target nucleic acids of interest, the direct capture of target nucleic acids can thereby facilitate amplification and detection of the target nucleic acids due the absence of potentially competitive non-target nucleic acids.

In certain embodiments, the microparticles have a diameter of about 0.5 to about 50 μm, e.g., about 0.5 μm, about 1.0 μm, about 1.5 μm, about 2.0 μm, about 5.0 μm, about 10.0 μm, about 20.0 μm, about 30.0 μm, about 40.0 μm or about 50.0 μm.

In certain embodiments, the microparticles can bind at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99% or at least about 100% of the nucleic acids, e.g., DNA or RNA, in the sample.

Additionally, or alternatively, subsequent to binding of nucleic acids to the microparticles, the microparticles are washed to remove contaminants and/or undesired material from the microparticles in the sample. In certain embodiments, the wash and elute step includes more than one wash. For example, but not by way of limitation, the wash process includes at least about 2 washes, at least 3 washes or at least 4 washes. In certain embodiments, the wash process includes at least about 2 washes. In certain embodiments, the wash process includes at least about 3 washes.

Additionally, or alternatively, at least one of the washes includes a detergent and/or a protein denaturant. Non-limiting examples of detergents are described herein.

Additionally, or alternatively, a wash solution for use in the present disclosure has a pH from about 5.5 to about 8.0, e.g., a pH of about 6.0, about 6.5, about 7.0 or about 7.5. In certain embodiments, the pH of a wash solution depends on the sample. For example, but not by way of limitation, the pH of a wash buffer can be 6.0. In certain embodiments, the pH of the wash buffer can be about 7.8. In certain embodiments, at least one of the washes is performed with water. For example, the first and second washes can be performed with water. In certain embodiments, the second and third washes can be performed with water. In certain embodiments, the first wash is performed with a lysis buffer and the second and third washes are performed with water.

Additionally, or alternatively, a volume of about 10 μl to about 500 μl of wash solution can be used for each wash. For example, but not by way of limitation, a volume of about 100 μl to about 500 μl of wash solution, e.g., about 250 μl, can be used for each wash.

In certain embodiments, the bound nucleic acids are eluted from the microparticles after the washing of the microparticles. In certain embodiments, an elution buffer is used to elute the nucleic acids from the microparticles to generate an eluate. In certain embodiments, the elution buffer comprises about 5 mM to about 10 mM phosphate and a pH of about 7.5 to about 9.0.

Additionally, or alternatively, the microparticles are incubated in the elution buffer for about 1 minute to about 10 minutes, e.g., for about 1 minute to about 9 minutes, for about 1 minute to about 7 minutes, for about 1 minute to about 6 minutes, for about 1 minute to about 5 minutes, for about 1 minute to about 4 minutes or for about 1 minute to about 3 minutes. In certain embodiments, the microparticles are incubated in the elution buffer for about 3 minutes for total nucleic acid capture. In certain embodiments, the microparticles are incubated in the elution buffer for about 2 minutes. In certain embodiments, the microparticles are incubated in the elution buffer for about 2 minutes for direct oligonucleotide capture. In certain embodiments, the microparticles are incubated in the elution buffer for about 192 seconds (i.e., about 3.2 minutes). In certain embodiments, the microparticles are incubated in the elution buffer at a temperature from about 60° C. to about 100° C., e.g., from about 70° C. to about 90° C. or from about 75° C. to about 85° C. In certain embodiments, the microparticles are incubated in the elution buffer at a temperature of about 80° C. The resultant eluate can then be used for a subsequent amplification reaction and is, in certain embodiments, capable of being used in any of the various nucleic acid analyses described herein to detect a pathogen or infectious agent.

### 2.5 Magnetic Transfer

As further disclosed herein, for example, and as embodied herein with reference to Example 2, FIGS. 15-17, and 42, the significant improvements in time to result and improved throughput per unit size, among other benefits, achieved by the methods and systems of the present disclosure are due, at least in part, to the incorporation of operational steps involving magnetic capture of magnetic particles. Such operational steps include but are not limited to: mixing; washing; and transfer of the magnetic particles. Existing methods of mixing and washing magnetic particles generally rely on mechanical agitation and magnetic mixing using moving permanent magnets. In certain embodiments, however, such mixing, washing, and/or transfer can be performed with a system incorporating at least one stationary electromagnet-based capture of magnetic particles. For example, but not by way of limitation, one or more of the distinct mixing or washing positions identified herein can be accomplished using a stationary electromagnet-based capture of magnetic particles. In addition, certain embodiments described herein can be performed with a system incorporating at least one stationary electromagnet-based capture of magnetic particles in connection with one or more transfer operations at one or more of the transfer positions within the systems described herein. As will be appreciated by those of skill in the art, the specific number, orientation, and operational assignment (e.g., mixing, washing, transfer, or elution) of the individual positions can be modified as desired and yet remain within the scope of the present disclosure.

The present disclosure contemplates the use, in certain embodiments, of moving permanent magnets and/or stationary electromagnets in a variety of positions to accomplish the appropriate mixing, washing, and transfer operations. For example, but not by way of limitation, moving permanent magnets and/or electromagnets can be positioned at right side, left side, and/or bottom side of a well. In addition, moving permanent magnets and/or electromagnets can be positioned with one side higher than the other. The moving permanent magnets and/or stationary electromagnets of the present disclosure can also be used in conjunction with a variety of well formats known in the art.

In certain embodiments, opposing moving permanent magnets and/or electromagnets can be arranged to alternately attract magnetic particles to the opposite sides of a wash well. However, the position, timing, power, and sequence of the moving permanent magnets and/or electromagnet activations is entirely flexible. For example, but not by limitation, stationary electromagnets can be used to partially collect magnetic particles and then allow the collected particles to drop to the bottom of the well. Alternatively, or additionally, magnetic particles can be collected on the sides of a well to facilitate mixing, washing, and/or transfer. In addition, magnetic particles can be slowly collected with lower power or magnetic particles can be more rapidly collected with higher power.

Distinct types of electromagnets can be contemplated for use in connection with the embodiments disclosed herein. For example, while DC electromagnets can be used, because DC electromagnets can magnetize magnetic particles, AC electromagnets can also be used in conjunction with or in place of DC electromagnets to avoid creating residual magnetism of the magnetic particles by varying the switching frequency.

The use of electromagnets in addition to or in lieu of permanent magnets (or other mixing, washing, or transfer aspects) provides several advantages. Mixing or transfer operations during sample preparation typically requires permanent magnets to be moved in and out of range of the magnetic particles. The use of electromagnets can eliminate motion mechanisms by simply turning on and off the electromagnets. In addition, magnetic particles can be moved from one well to another by successively turning on and off adjacent magnets within an array. Incorporating a stationary electromagnet-based particle capture approach can also eliminate one or more disposable per test, thus reducing the amount of solid waste being generated. The use of stationary electromagnets also eliminates the need for specific volume requirements and disposable coverings for the wells or moving permanent magnet when transferring magnetic particles from one well to another. The instant aspect can also transfer magnetic particles between wells using moving electromagnets adjacent to the side of the wash vessel, but not touching the magnetic particles or liquid within wells, minimizing magnetic particle loss and liquid carryover from source to destination wells, thus maximizing assay performance. E.g., by providing eluate with fewer contaminates and more nucleic acids from the sample.

In some embodiments, transfer can be accomplished using moveable magnets below the wells to slide the microparticles internal channels at the bottom of wells to collect, transfer and release the microparticles. In certain embodiments stationary electro-magnets, e.g., selectively turning on/off adjacent magnets to achieve magnetic particle movement can be employed. Other methods of transferring microparticles such as in inverse particle processing can also be used.

### 2.6 Pre-Treatment Process

The present disclosure contemplates the use, in certain embodiments, of treating the sample with a pre-treatment process. For example, but not by way of limitation, a whole blood sample can be treated with a pre-treatment lysis process to generate a lysed whole blood sample prior to further sample processing in the presence of microparticles.

### 2.6.1 Whole Blood Pre-Treatment

In accordance with an aspect of the disclosed subject matter, the pre-treatment process can be a pre-treatment lysis process. For example, the pre-treatment process can be performed on samples (e.g., whole blood) prior to the lysis process (e.g., lysis with a sample lysis buffer in the presence of microparticles) and wash process described above. In certain embodiments, the pre-treatment lysis process includes contacting a whole blood sample with lysis buffer, e.g., an RBC lysis solution. In certain embodiments, the pre-treatment lysis process includes contacting a whole blood sample with lysis buffer, e.g., at a ratio of whole blood sample to lysis buffer of about 1:1.

In certain embodiments, the pre-treatment process, e.g., pre-treatment lysis process, can be performed on a sample transport in the sample preparation area. For example, in certain embodiments, the pre-treatment process can be carried out in vessels on the sample transport as the vessels are continually transported along a transport path of the sample transport between a sample dispense position and a sample capture and transfer position. In certain embodiments, a pre-treatment process and lysis process can be performed on the sample transport. Additionally or alternatively, and as described further herein, In certain embodiments, a pre-treatment process, onboard pooling process, and lysis process can be performed on the sample transport. For example, In certain embodiments, after a sample has completed a pre-treatment process in a vessel on the sample transport, the sample can be aspirated from the vessel and dispensed into another vessel on the sample transport, e.g., at the initial sample dispense position, to continue with another aspect of a sample preparation process. For example after pre-treatment, the sample can be dispensed into a vessel at the initial dispense position to begin a lysis process.

For purpose of example and as embodied herein, the pre-treatment process can be performed on a sample preparation carousel, e.g., a lysis carousel. Exemplary operations regarding the pre-treatment process are provided in Table A below.

TABLE A

| | | Time |
|---|---|---|
| Pos. | Function | (seconds) |
| L1 | Load Lysis Tube in Carousel | |
| | Load Transfer Tip in Carousel | |
| L2 | Dispense Lysis Buffer | |
| L3 | | |
| L4 | Dispense Sample | 24 |
| | Dispose of Tip | |
| L5-L16 | Incubation and Mixing | 288 |
| L17 | Aspirate from L17 and return | 24 |
| | Sample to L4 | |
| | Dispose of Tip | |
| L18-L19 | | |
| L20 | Aspirate Lysis Contents | |
| L21 | Transfer Lysis Tube to Waste | |

In the present embodiment, exemplary position L1 corresponds to the loading of the lysis tube on the sample preparation carousel, e.g., lysis carousel 6411. In certain embodiments, loading is accomplished by known “Pick & Place” strategies from a loadable stack. L2 corresponds to a lysis buffer dispensing position. L4 corresponds to a sample dispensing position. Sample dispensed at this position can be dispensed via known “Sip & Spit” strategies from sample containers.

Exemplary positions L5-L16 correspond to incubation and mixing positions. For example, incubation and mixing positions L5-L16 can incorporate the use of resistive heaters, carousel movement, pop-up mixers, lock step transfers, and/or time priority scheduling. In certain embodiments, positions L5-L16 can incorporate incubation in one or more sample lysis buffer. In certain embodiments, the sample can be incubated and mixed for about 3 minutes to about 6 minutes, about 4 minutes to about 6 minutes, or about 5 minutes to about 6 minutes. In certain embodiments, the sample can be incubated and mixed for about 3 minutes, about 4 minutes, about 5 minutes, or about 6 minutes. In certain embodiments, the sample can be incubated and mixed for about 288 seconds, or about 4.8 minutes.

Exemplary position L17 corresponds to aspiration of the sample from the sample preparation carousel and returning the sample to the initial sample dispense position (e.g., exemplary position L4). As noted above, in certain embodiments, after completing a pre-treatment process the sample can be transferred to another vessel on the sample transport to initiate another aspect of the sample preparation process. For example, and as embodied herein, the sample can be returned to initial dispense position to begin a lysis process. Exemplary position L20 corresponds to aspirating any remaining contents from the pre-treatment process, e.g., any remaining lysis contents. Exemplary position L21 corresponds to transferring the lysis tube to waste.

In certain embodiments, the time to perform nucleic acid analysis (e.g., time to result or TTR) can include the time required to perform a pre-treatment process. In certain embodiments, sample pre-treatment process can start with the initial aspiration of a sample for nucleic acid analysis. For example, in certain embodiments, the pre-treatment process can start at the aspiration of the sample from a sample vessel for dispensing on the sample preparation carousel (e.g., for dispensing at exemplary position L4) and ends with transferring the pre-treated sample to another vessel on the sample transport to initiate another aspect of the sample preparation process, e.g., dispensing of the pre-treated sample to a lysis tube that includes the lysis buffer with microparticles (e.g., aspiration of the pre-treated sample from exemplary position L17 and dispensing of the pre-treated sample to exemplary position L4). Alternatively, the pre-treatment process can start at the aspiration of the sample from a sample vessel for dispensing on the sample preparation carousel (e.g., at exemplary position L4) and ends with the completion of the incubation of the sample in the lysis buffer (e.g., at exemplary position L16).

In certain embodiments, the pre-treatment process, e.g., pre-treatment lysis process, can be completed in about 3 minutes to about 7 minutes, about 4 minutes to about 6 minutes, or about 5 minutes to about 6 minutes. In certain embodiments, the pre-treatment process is about 3 minutes, about 4 minutes, about 5 minutes, about 6 minutes, or about 7 minutes. In certain embodiments, the pre-treatment process can be about 336 seconds, or about 5.6 minutes. For example and with reference to Table A, the pre-treatment process can begin with dispensing of a sample into a vessel at position L4, which can be performed in about 24 seconds. The pre-treatment process can further include about 288 seconds of incubation and mixing. Additionally, the pre-treatment process can include aspirating the pre-treated sample from the vessel, e.g., lysis tube, on the sample transport and dispensing the pre-treated sample into another vessel on the sample transport, which can be performed in about 24 seconds. In certain embodiments, the pre-treatment process can be about 312 seconds, or about 5.2 minutes, e.g., if the time to aspirate the pre-treated sample from the vessel, e.g., lysis tube, on the sample transport and dispense the pre-treated sample into another vessel on the sample transport is not included, e.g., if the time to perform the procedures corresponding to exemplary position L17 is not included.

In certain aspects, the time to perform the procedures corresponding to exemplary positions L1, L2, and L4 is not considered in the calculation of TTR. In certain aspects, the time to perform the procedures corresponding to exemplary positions L1, L2, and L4 is not considered in the calculation of the duration of the pre-treatment process.

In certain aspects, the time to perform the procedures corresponding to exemplary positions L1 and L2 is not considered in the calculation of TTR. In certain aspects, the time to perform the procedures corresponding to exemplary positions L1 and L2 is not considered in the calculation of the duration of the pre-treatment process.

In certain aspects, the time to perform the procedures corresponding to exemplary positions L17, L20, and L21 is not considered in the calculation of TTR. In certain aspects, the time to perform the procedures corresponding to exemplary positions L17, L20, and L21 is not considered in the calculation of the duration of the pre-treatment process.

In certain aspects, the time to perform the procedures corresponding to exemplary positions L20 and L21 is not considered in the calculation of TTR. In certain aspects, the time to perform the procedures corresponding to exemplary positions L20 and L21 is not considered is not considered in the calculation of the duration of the pre-treatment process.

### 3. Nucleic Acid Amplification Aspects

In accordance with the disclosed subject matter, and as embodied herein, the methods and systems disclosed herein for rapid nucleic acid testing of samples include unique nucleic acid amplification aspects to amplify target nucleic acid isolated from samples. In certain embodiments, the methods and systems of the disclosed herein for rapid screening of donor blood include unique nucleic acid amplification aspects to amplify target nucleic acid isolated from a donor sample. For example, but not limitation, reference is now made to nucleic acid amplification methods and system components as contemplated for the methods and systems of the present disclosure.

Typically, nucleic acid amplification is employed to increase the number of a target nucleic acid in the sample, e.g., to thereby facilitate detection of the target nucleic acid. As embodied herein, the nucleic acid amplification methods and system components can be configured to amplify a target nucleic acid using any of a variety or combination of suitable amplification techniques.

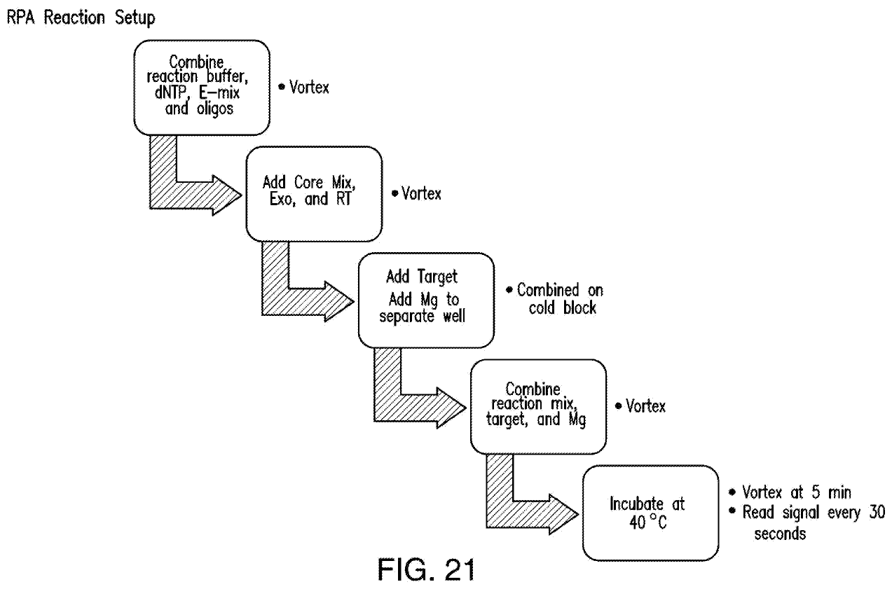

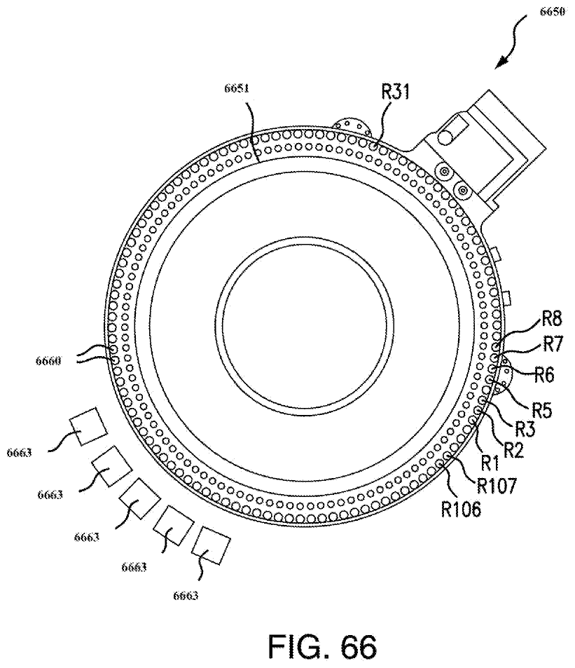

As further disclosed herein, and as embodied herein with reference to FIGS. 1, 2A-2B, 9-10, 66 and 79, after the sample is prepared, e.g., nucleic acids are isolated from the sample, the isolated nucleic acids can be amplified. In accordance with one aspect of the disclosed subject matter, e.g., as embodied in FIG. 66, the amplification methods and system components include contacting the isolated nucleic acids with the amplification oligonucleotides, e.g., forward and reverse primer oligonucleotides, and probes as described herein to form a reaction mixture. The reaction mixture is then placed under amplification conditions. The term “amplification conditions,” as used herein, refers to conditions that promote annealing and/or extension of the amplification oligonucleotides. In certain embodiments, such conditions include contacting the isolated nucleic acids with an “E-Mix” or a “Core Mix”, e.g., as embodied in FIG. 2B and FIG. 21. Typically, an E-Mix is a solution comprising ATP, Phosphocreatine, and buffer. In contrast, a Core Mix typically comprises a collection of proteins necessary to amplify a nucleic acid target. For example, but not limitation, a typical Core Mix for an RPA amplification can comprise gp32, uvsX, uvsY, and a Polymerase. In certain embodiments, such conditions include contacting the isolated nucleic acids with a “MasterMix.” As used herein, a MasterMix refers to a solution comprising all of the components, e.g., nucleotide triphosphates, polymerases, primers, and probes, necessary to amplify a target nucleic acid for subsequent detection, except an activator, which can be separately provided to initiate amplification, e.g., as illustrated in the exemplary embodiment depicted in FIG. 66. For example, but not by way of limitation, FIG. 66 illustrates an embodiment where an activator is initially dispensed into an amplification vessel (at position R4), the eluate comprising the isolated nucleic acid is then dispensed into the amplification vessel (at position R5) and finally the Master Mix is dispensed into the amplification vessel (at position R6), although alternative orders of addition are contemplated within the scope of the methods and systems described herein. Amplification conditions are well-known in the art and depend on the amplification method selected. In accordance with the disclosed subject matter, amplification conditions encompass a wide range of reaction conditions including, but not limited to, temperature and/or temperature cycling, buffer, salt, ionic strength, pH, and the like.

Additionally, or alternatively, and in accordance with another aspect of the disclosed subject matter, the amplification methods and system components of the present disclosure include the use of rapid amplification strategies having a duration of about 1 minute to about 60 minutes, about 5 minutes to about 60 minutes, about 8 minutes to about 60 minutes, or in about 8 minutes to about 50 minutes, or in about 8 minutes to about 40 minutes, or in about 8 minutes to about 35 minutes, or in about 8 minutes to about 30 minutes, or in about 8 minutes to about 25 minutes, or about 8 minutes to about 20 minutes, about 1 minute to about 22 minutes, about 5 minutes to about 22 minutes, about 8 minutes to about 22 minutes, about 1 minute to about 20 minutes, about 5 minutes to about 20 minutes, about 8 minutes to about 20 minutes, or about 8 minutes to about 15 minutes from the addition of the reagents sufficient to initiate amplification of a sample of eluted nucleic acids if the targeted nucleic acid(s) is present. For purpose of illustration not limitation, the amplification methods and systems components of the present disclosure can be performed on an amplification and detection system. As embodied herein, the subsystem includes a rotating carousel, e.g., as shown in FIG. 78.

Additionally, or alternatively, and in accordance with another aspect of the disclosed subject matter, the amplification methods and system components of the present disclosure include amplifying a pathogen or an infectious agent nucleic acid sequence in the sample using any suitable nucleic acid sequence amplification method known in the art, e.g., polymerase chain reaction (PCR), reverse-transcriptase PCR (RT-PCR), real-time PCR, transcription-mediated amplification (TMA), rolling circle amplification, nucleic acid sequence-based amplification (NASBA), strand displacement amplification (SDA), Transcription-Mediated Amplification (TMA), Single Primer Isothermal Amplification (SPIA), Helicase-dependent amplification (HDA), Loop mediated amplification (LAMP), Recombinase-Polymerase Amplification (RPA), Nicking Enzyme Amplification Reaction (NEAR) and ligase chain reaction (LCR). In certain embodiments, amplification is performed using isothermal amplification, for example, but not by way of limitation, isothermal amplification via RPA or NEAR. In certain embodiments, the isothermal amplification method is RPA. In certain embodiments, the isothermal amplification method is NEAR. Additional non-limiting disclosure regarding isothermal amplification methods is provided in Oliveira et al., Frontiers in Sensors 2:752600 (2021), the contents of which is incorporated herein by reference in its entirety.

### 3.1 RPA

In accordance with the disclosed subject matter, and as embodied herein, the methods and systems of the disclosed subject matter for nucleic acid testing of a sample, e.g., rapid screening of donor blood, can include amplification of a target nucleic acid, e.g. infectious agent nucleic acid sequences, using Recombinase-Polymerase Amplification (RPA). RPA relies on the properties of recombinase and related protein components to invade double-stranded nucleic acids with single stranded homologous nucleic acids permitting sequence specific priming of nucleic acid polymerase reactions.

Typically, RPA nucleic acid amplification reactions exploit enzymes known as recombinases, which form complexes with oligonucleotide primers and pair the primers with their homologous sequences in duplex nucleic acids. A single-stranded nucleic acid binding (SSB) protein binds to the displaced nucleic acid strand and stabilizes the resulting loop. Nucleic acid amplification is then initiated from the primer, but only if the target sequence is present. Once initiated, the amplification reaction progresses rapidly, so that starting with just a few target copies of nucleic acid, the highly specific amplification reaches detectable levels within minutes.

In some embodiments, the RPA reaction contains a mixture of a recombinase, a single-stranded binding protein, a polymerase, dNTPs, ATP, a primer, and a template nucleic acid. In some embodiments, an RPA reaction can include one or more of the following (in any combination): at least one recombinase; at least one single-stranded DNA binding protein; at least one DNA polymerase; dNTPs; a crowding agent; a buffer; a reducing agent; ATP or an ATP analog; at least one recombinase loading protein; a first primer and optionally a second primer; a probe; a reverse transcriptase; and a template nucleic acid molecule, e.g., a single-stranded (e.g., RNA) or double stranded nucleic acid. In some embodiments, the RPA reaction can contain, e.g., a reverse transcriptase. In certain embodiments, the RPA reaction does not include a reverse transcriptase. An exemplary RPA reaction vessel is disclosed in U.S. Pat. No. 9,535,082 and is herein incorporated by reference in its entirety.

In some embodiments, in a first step, a first and a second single stranded nucleic acid primer is contacted with a recombinase (e.g., UvsX), a recombinase loading agent (e.g. UvsY) and a single strand DNA binding protein (e.g., gp32) to form a first and a second nucleoprotein primer. The single stranded nucleic acid primers are specific for and are complementary to the target nucleic acid molecule. In the second step, the first nucleoprotein primer is contacted to the double stranded target nucleic acid molecule to create a first D loop structure at a first portion of the double stranded target nucleic acid molecule (Step 2a). Further, the second nucleoprotein primer is contacted to the double stranded target nucleic acid molecule to create a second D loop structure at a second portion of the double stranded target nucleic acid molecule (Step 2b). The D loop structures are formed such that the 3′ ends of the first nucleic acid primer and said second nucleic acid primer are oriented toward each other on the same double stranded target nucleic acid molecule without completely denaturing the target nucleic acid molecule. It should be noted that step 2a and step 2b can be performed in any order or simultaneously.

In a D loop structure, the primer is hybridized to one strand of the double stranded target nucleic acid molecule to form a double stranded structure. The second strand of the target nucleic acid molecule is displaced by the primer. The structure resembles a capital D where the straight part of the D represents the double stranded part of the structure and the curved part of the D represents the single stranded displaced second strand of the target nucleic acid.

In the third step, the 3′ end of the first and the second nucleoprotein primer is extended with one or more polymerases capable of strand displacement synthesis and dNTPs to generate a first and second double stranded target nucleic acid molecule and a first and second displaced strand of nucleic acid. The first and second double stranded target nucleic acid molecules may serve as target nucleic acid molecules in step two during subsequent rounds of amplification.

Steps two and step three are repeated until a desired degree of amplification of the target nucleic acid is achieved.

During the amplification process described above, the first and second displaced strand of nucleic acid may hybridize to each other after step (c) to form a third double stranded target nucleic acid molecule.

In certain embodiments, an RPA reaction for use in the present disclosure comprises combining the reaction buffer, “E-mix”, dNTPs and oligos (e.g., as a first step). In certain embodiments, the “Core Mix,” the exo probe, gp32, uvsX, uvsY, polymerase and reverse transcriptase is added to the reaction (e.g., as a second step). The isolated nucleic acids and an activator, e.g., magnesium, are then added to the reaction (e.g., as a third step), followed by the incubation of the reaction at 40° C. (e.g., as a fourth step). In certain embodiments, the recombinase (e.g., UvsX), recombinase loading agent (e.g., UvsY) and single strand DNA binding protein (e.g., gp32) may be derived from a myoviridae phage. The myoviridae phage may be, for example, T4, T2, T6, Rb69, Aehl, KVP40, Acinetobacter phage 133, Aeromonas phage 65, cyanophage P-SSM2, cyanophage PSSM4, cyanophage S-PM2, Rbl4, Rb32, Aeromonas phage 25, Vibrio phage nt-1, phi-1, Rbi6, Rb43, Phage 31, phage 44RR2.8t, Rb49, phage Rb3, or phage LZ2. In some embodiments, the combination of Rb69 UvsX, Rb69 UvsY and Rb69 gp32 may be used. In another preferred embodiment, the combination of Aehl UvsX, Aehl UvsY and Rb69 gp32 may be used. In another preferred embodiment, the combination of T4 UvsX, T4 UvsY and Rb69 gp32 may be used. In another preferred embodiment, the combination of T4 UvsX, Rb69 UvsY and T4 gp32 may be used.

Further, in any of the processes of this disclosure, the recombinase (e.g., UvsX), recombinase loading agent (e.g., UvsY) and single strand DNA binding protein (e.g., gp32) can each be native, hybrid or mutant proteins from the same or different myoviridae phage sources. A native protein may be a wildtype or natural variant of a protein. A mutant protein (also called a genetically engineered protein) is a native protein with natural or manmade mutations such as insertions, deletions, substitutions, or a combination thereof, that are at the N terminus, C terminus, or interior (between the N terminus and the C terminus). A hybrid protein (also called a chimeric protein) comprises sequences from at least two different organisms. For example, a hybrid UvsX protein may contain an amino acid from one species (e.g., T4) but a DNA binding loop from another species (e.g., T6). The hybrid protein may contain improved characteristics compared to a native protein. The improved characteristics may be increased or more rapid RPA amplification rate or a decreased or more controllable RPA amplification rate.

In some embodiments, the recombinase (e.g., UvsX) may be a mutant UvsX. In a preferred embodiment, the mutant UvsX is an Rb69 UvsX comprising at least one mutation in the Rb69 UvsX amino acid sequence, wherein the mutation is selected from the group consisting of (a) an amino acid which is not histidine at position 64, a serine at position 64, the addition of one or more glutamic acid residues at the C-terminus, the addition of one or more aspartic acid residues at the C-terminus, and a combination thereof. In another preferred embodiment, the mutant UvsX is a T6 UvsX having at least one mutation in the T6 UvsX amino acid sequence, wherein the mutation is selected from the group consisting of (a) an amino acid which is not histidine at position 66; (b) a serine at position 66; (c) the addition of one or more glutamic acid residues at the C-terminus; (d) the addition of one or more aspartic acid residues at the C-terminus; and (e) a combination thereof.

In certain embodiments, the RPA process is performed in the presence of a crowding agent. The crowding agent may be selected from the group comprising polyethylene glycol (e.g., PEG1450, PEG3000, PEG8000, PEG10000, PEG14000, PEG15000, PEG20000, PEG250000, PEG30000, PEG35000, PEG40000, PEG compound with molecular weight between 15,000 and 20,000 daltons), polyethylene oxide, polyvinyl alcohol, polystyrene, Ficoll, dextran, PVP, albumin and a combination thereof. In some embodiments, the crowding agent has a molecular weight of less than 200,000 daltons. Further, the crowding agent may be present in an amount of about 0.5% to about 15% weight to volume (w/v). In certain embodiments, the crowding agent can be present in an amount of about 1% to about 10% w/v.

In some embodiments, the RPA processes are performed with a polymerase which is a large fragment polymerase. The large fragment polymerase may be selected from the group consisting of E. Coli Pol I, Bacillus subtilis Pol I, Staphylococcus aureus Pol I, and homologues thereof. In certain embodiments, the RPA processes are performed in the presence of about 0.01 mg/mL to about 0.5 mg/mL DNA Polymerase, e.g., about 0.08 mg/mL to about 0.2 mg/mL DNA Polymerase. In certain embodiments, the RPA processes are performed in the presence of about 10 units/mL to about 10,000 units/mL of DNA Polymerase, e.g., about 500 units/mL to about 5,000 units/mL of DNA Polymerase.

In some embodiments, the RPA processes are performed in the presence of heparin. Heparin may serve as an agent to reduce the level of non-specific primer noise, and to increase the ability of E. coli exonuclease III or E. Coli exonuclease IV to rapidly polish 3′ blocking groups or terminal residues from recombination intermediates.

In some embodiments, the RPA processes are performed with a blocked primer. A blocked primer is a primer which does not allow elongation with a polymerase. Where a blocked primer is used, an unblocking agent is also used to unblock the primer to allow elongation. The unblocking agent may be an endonuclease or exonuclease which can cleave the blocking group from the primer. In some embodiments, unblocking agents include E. coli exonuclease III and E. coli endonuclease IV. In certain embodiments, the unblocking agent is E. coli exonuclease III. In certain embodiments, the unblocking agent is E. coli endonuclease IV.

In certain embodiments, the RPA processes are performed in the presence of two or more primers, e.g., (i) at least one or more forward primers, (ii) at least one or more reverse primers or (iii) at least one or more forward and reverse primers, and/or at least one or more probes. In certain embodiments, the RPA processes are performed in the presence of at least three primers. In certain embodiments, the RPA processes are performed in the presence of at least two probes. In certain embodiments, the RPA processes are performed in the presence of about 1 nM to about 1,000 nM of one or more primers and/or probes, e.g., about 10 nM to about 500 nM of one or more primers and/or probes. In certain embodiments, the RPA processes are performed in the presence of about 1 nM to about 1,000 nM of one or more primers, e.g., about 10 nM to about 500 nM of one or more primers. In certain embodiments, the RPA processes are performed in the presence of about 1 nM to about 1,000 nM of a forward primer, e.g., about 10 nM to about 500 nM of a forward primer. In certain embodiments, the RPA processes are performed in the presence of about 1 nM to about 1,000 nM of a reverse primer, e.g., about 10 nM to about 500 nM of a reverse primer. In certain embodiments, the RPA processes are performed in the presence of about 1 nM to about 1,000 nM of one or more probes, e.g., about 10 nM to about 500 nM of one or more probes, e.g., detection probes.

In certain embodiments, the RPA processes are performed in the presence of about 1 mM to about 25 mM divalent manganese ions, e.g., about 1 mM to about 20 mM, about 1 mM to about 10 mM or about 1 mM to about 3 mM divalent manganese ions. In some embodiments, the manganese ions replace the magnesium ions and the reaction may be performed with or without magnesium.

Furthermore, in some embodiments, UvsY is omitted. That is, any of the RPA reactions of this disclosure may be performed in the absence of UvsY.

In certain embodiments, the reverse transcriptase is omitted from the RPA reaction. For example, but not by way of limitation, any of the RPA reactions of this disclosure can be performed in the absence of a reverse transcriptase. In certain embodiments, an RPA reaction of the presence disclosure is performed in the absence of a reverse transcriptase if the target nucleic acid to be analyzed is DNA.

In certain embodiments, only one of the nucleic acid primers is coated with recombinase/recombinase loading agent/single stranded DNA binding protein. That is, an RPA may be performed with one primer which is uncoated and one primer which is coated with any one or a combination of recombinase, recombinase loading agent, and single stranded DNA binding protein.

For examples of RPA amplification strategies useful in connection the methods and systems of the present disclosure, see U.S. Pat. Nos. 7,270,981; 8,460,875; 7,399,590; 7,435,561; 7,485,428; 7,666,598; 7,763,427; 7,759,061; 8,022,914; 8,030,000; 8,229,226; 8,426,134; 8,580,507; 8,945,845; 9,057,097; 9,157,127; 9,340,825; 9,469,867; 9,663,820; 9,896,719; 9,932,577; 10,329,603; 10,329,602; 10,538,760; 8,017,339; 8,574,846; 8,962,255; 10,036,057; 8,071,308; 10,093,908; 10,947,584; and 8,637,253, and U.S. patent application Ser. Nos. 15/099,754; 14/705,150; and Ser. No. 16/155,133, each of which is incorporated herein by reference in its entirety. Additional RPA amplification strategies useful in connection the methods and systems of the present disclosure are disclosed in Lobato and O'Sullivan, Trends in Analytical Chemistry 98:19-35 (2018), and Daher et al., Clinical Chemistry 62(7):947-958 (2016), the contents of each of which are incorporated herein by reference in its entirety.

Additionally, or alternatively, RPA nucleic acid amplification can be employed using RNA as an initial template, e.g., to amplify a target nucleic acid derived from an RNA virus, by using reverse transcriptase to first produce a DNA copy of the RNA template after which the DNA copy can be subjected to RPA-based nucleic acid amplification. Performing RPA with RNA templates is typically referred to in the art as Reverse Transcriptase RPA or RT-RPA. In certain embodiments, the reverse transcriptase used in the methods and systems of the present disclosure can be selected from: OmniScript (Qiagen), SensiScript (Qiagen), MonsterScript (Epicentre), Transcriptor (Roche), HIV RT (Ambion), Superscript III (Invitrogen), ThermoScript (Invitrogen), Thermo-X (Invitrogen), ImProm II (Promega) and EIAV-RT. In certain embodiments, the reverse transcriptase is EIAV-RT.

In certain embodiments, the ATP or analog thereof can be used at a concentration of about 1 and about 10 mM. Non-limiting examples an ATP analog include ATP-γ-S, ATP-β-S and ddATP.

In certain embodiments, the following reagents can be employed for performing an RPA reaction: Tris-HCl, DTT, Potassium Acetate, a crowding agent (e.g., PEG), dNTPs, ATP, Phosphocreatine, Glycerol, Creatine Kinase, UvsX, UvsY, DNA polymerase, GP32, Exonuclease III, BSA, an activator (e.g., Magnesium, e.g., Mg Acetate), and EIAV. In certain embodiments, additional reagents can be employed, including but not limited to, forward primers, reverse primers, probes and ROX reference dyes.

Additionally, or alternatively, and in accordance with another aspect of the disclosed subject matter the following reagents can be employed at the following concentrations: about 5 mM to about 100 mM Tris-HCl at pH of about 6.5-9.0, e.g., 8.3; about 5 mM to about 10 mM DTT; about 50 mM to about 100 mM Potassium Acetate; about 2% to about 10% of a crowding agent, e.g., PEG; about 1 mM to about 5 mM dNTPs; about 1 mM to about 10 mM of ATP, e.g., about 2 mM to about 5 mM ATP; about 20 mM to about 100 mM Phosphocreatine, e.g., about 40 mM to about 100 mM Phosphocreatine; about 5 mM to about 40 mM Mg Acetate, e.g., about 10 mM to about 40 mM Mg Acetate; about 0.01 mg/mL to about 10 mg/mL BSA; about 5% to about 10% Glycerol; about 0.01 mg/mL to about 0.5 mg/mL Creatine Kinase, e.g., about 0.1 mg/mL to about 0.5 mg/mL Creatine Kinase; about 0.1 mg/mL to about 1.0 mg/mL UvsX, e.g., about 0.3 mg/mL to about 1.0 mg/mL UvsX; about 0.01 mg/mL to about 0.25 mg/mL UvsY, e.g., about 0.09 mg/mL to about 0.25 mg/mL UvsY; about 0.01 mg/mL to about 0.5 mg/mL DNA Polymerase, e.g., about 0.08 mg/mL to about 0.2 mg/mL DNA Polymerase; about 0.1 mg/mL to about 2.0 mg/mL GP32, e.g., about 0.4 mg/mL to about 0.8 mg/mL GP32; about 0.01 mg/mL to about 0.5 mg/mL Exonuclease III, e.g., about 0.1 mg/mL to about 0.5 mg/mL Exonuclease III; and about 0.5 μg/mL to about 20.0 μg/mL equine infectious anemia virus reverse transcriptase (EIAV-RT), e.g., about 0.5 μg/mL to about 1.5 μg/mL EIAV-RT. In certain embodiments, additional reagents can be employed, including but not limited to, forward primers, reverse primers, probes and ROX reference dyes.

In certain embodiments, the reagents for use in the present disclosure, e.g., in an RPA reaction, can have the concentrations provided in Table 16 or Table 17. For example, but not by way of limitation, a singleplex reaction for detecting a pathogen or an infectious agent, e.g., Parvovirus B19 or HAV, can comprise the reagents at the concentrations provided in Table 17. In certain embodiments, a multiplex reaction for detecting Parvovirus B19 and HAV can comprise the reagents at the concentrations provided in Table 17. In certain embodiments, a singleplex reaction can comprise the following reagents employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 1.8 mM, ATP at 2.5 mM, Phosphocreatine at 50 mM, Forward Primer at 420 nM, Reverse Primer at 420 nM, Exo Probe at 120 nM, ROX reference dye at 15 nM, Glycerol at 6.5%, Creatine Kinase at 0.1 mg/ml, UvsX at 0.3 mg/ml, UvsY at 0.09 mg/ml, DNA Polymerase at 0.0798 mg/ml, Gp32 at 0.48 mg/ml, Exonuclease III at 0.1 mg/ml, Bovine Serum Albumin (BSA) at 0.02 mg/ml, EIAV Reverse Transcriptase at 0.0008 mg/ml and Magnesium Acetate at 14 mM. In certain embodiments, a multiplex reaction can comprise the following reagents employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 1.8 mM, ATP at 3.5 mM, Phosphocreatine at 50 mM, Parvovirus B19 Forward Primer at 147.5 nM, Parvovirus B19 Reverse Primer at 147.5 nM, Parvovirus B19 Exo Probe at 85 nM, HAV Forward Primer at 393.8 nM, HAV Reverse Primer at 236.3 nM, HAV Exo Probe at 120 nM, ROX reference dye at 15 nM, Glycerol at 8.4%, Creatine Kinase at 0.035 mg/ml, UvsX at 0.42 mg/ml, UvsY at 0.126 mg/ml, DNA Polymerase at 0.168 mg/ml, Gp32 at 0.672 mg/ml, Exonuclease III at 0.1 mg/ml, Bovine Serum Albumin (BSA) at 0.02 mg/ml, EIAV Reverse Transcriptase at 0.0016 mg/ml and Magnesium Acetate at 14 mM.

In certain embodiments, a singleplex reaction for detecting a pathogen or infectious agent, e.g., HIV-1, HIV-2, HCV or HBV, can comprise the reagents at the concentrations provided in Table 16. In certain embodiments, a multiplex reaction for detecting HIV-1, HIV-2, HCV and/or HBV can comprise the reagents at the concentrations provided in Table 16. In certain embodiments, a singleplex reaction can comprise the following reagents employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 1.8 mM, ATP at 2.5 mM, Phosphocreatine at 50 mM, Forward Primer at 420 nM, Reverse Primer at 420 nM, Exo Probe at 120 nM, ROX reference dye at 15 nM, Glycerol at 6.5%, Creatine Kinase at 0.1 mg/ml, UvsX at 0.3 mg/ml, UvsY at 0.09 mg/ml, DNA Polymerase at 0.0798 mg/ml, Gp32 at 0.48 mg/ml, Exonuclease III at 0.1 mg/ml, Bovine Serum Albumin (BSA) at 0.02 mg/ml, EIAV Reverse Transcriptase at 0.0008 mg/ml and Magnesium Acetate at 14 mM. In certain embodiments, a multiplex reaction, e.g., for detecting HIV-1 and HBV, can comprise the following reagents employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 2.7 mM, ATP at 3.5 mM, Phosphocreatine at 50 mM, HIV-1 INT Forward Primer at 157.50 nM, HIV-1 INT Reverse Primer at 236.37 nM, HIV-1 INT Exo Probe at 90 nM, HIV-1 LTR Forward Primer at 39.37 nM, HIV-1 LTR Reverse Primer at 39.37 nM, HIV-1 INT Exo Probe at 22.5 nM, HBV Forward Primer at 86.13 nM, HBV Reverse Primer at 86.13 nM, HBV Exo Probe at 90 nM, ROX reference dye at 45 nM, Glycerol at 8.4%, Creatine Kinase at 0.1 mg/ml, UvsX at 0.375 mg/ml, UvsY at 0.0675 mg/ml, DNA Polymerase at 0.1396 mg/ml, Gp32 at 1.2 mg/ml, Exonuclease III at 0.1 mg/ml, Bovine Serum Albumin (BSA) at 0.02 mg/ml, EIAV Reverse Transcriptase at 0.0016 mg/ml and Magnesium Acetate at 14 mM. In certain embodiments, a multiplex reaction, e.g., for detecting HIV-1 and HBV, can comprise the following reagents employed at the following concentrations: Tris-HCl 1M, pH 8.3 at 50 mM, DTT at 5 mM, Potassium Acetate at 100 mM, 20% Polyethylene glycol at 5.5%, 100 mM dNTPs at 2.7 mM, ATP at 3.5 mM, Phosphocreatine at 50 mM, HIV-1 Forward Primer at 157.50 nM, HIV-1 Reverse Primer at 236.37 nM, HIV-1 Exo Probe at 90 nM, HBV Forward Primer at 86.13 nM, HBV Reverse Primer at 86.13 nM, HBV Exo Probe at 90 nM, ROX reference dye at 45 nM, Glycerol at 8.4%, Creatine Kinase at 0.1 mg/ml, UvsX at 0.375 mg/ml, UvsY at 0.0675 mg/ml, DNA Polymerase at 0.1396 mg/ml, Gp32 at 1.2 mg/ml, Exonuclease III at 0.1 mg/ml, Bovine Serum Albumin (BSA) at 0.02 mg/ml, EIAV Reverse Transcriptase at 0.0016 mg/ml and Magnesium Acetate at 14 mM.

Additionally, or alternatively, and in accordance with another aspect of the disclosed subject matter the RPA reaction volume will be about 50 μL to about 100 ptL.

Additionally, or alternatively, and in accordance with another aspect of the disclosed subject matter, the RPA reaction temperature is between about 20° C. to about 50° C., about 20° C. to about 40° C., about 20° C. to about 30° C., or about 37° C. to about 42° C. In certain embodiments, the RPA reaction temperature is about 40° C.

Additionally, or alternatively, and in accordance with another aspect of the disclosed subject matter, the RPA reaction time is about 10 minutes to about 30 minutes, or in about 10 minutes to about 25 minutes, or about 10 minutes to about 20 minutes, or even about 10 minutes to about 15 minutes from the addition of the reagents sufficient to initiate RPA amplification. In certain embodiments, the RPA reaction time is about 1 minute to about 20 minutes, about 5 minutes to about 20 minutes, about 8 minutes to about 20 minutes, about 1 minute to about 10 minutes or about 5 minutes to about 10 minutes from the addition of the reagents sufficient to initiate RPA amplification. In certain embodiments, the RPA reaction time is about 1 minute. In certain embodiments, the RPA reaction time is about 5 minutes. In certain embodiments, the RPA reaction time is about 20 minutes. In certain embodiments, the RPA reaction is sufficient to obtain a result.

### 3.2 NEAR

In accordance with the disclosed subject matter, and as embodied herein, the methods and systems disclosed herein for rapid screening of donor blood can include isothermal amplification methods that rely on nicking and extension amplification reactions (NEAR) to amplify shorter sequences in a quicker timeframe than traditional amplification reactions. These methods can include, for example, reactions that use only two amplification oligonucleotides, one or two nicking enzymes, and a polymerase, under isothermal conditions.

Typically, in nicking and extension amplification, a target nucleic acid sequence, having a sense and antisense strand, is contacted with a pair of amplification oligonucleotides. The first amplification oligonucleotide comprises a nucleic acid sequence comprising a recognition region at the 3′ end that is complementary to the 3′ end of the target sequence antisense strand, a nicking enzyme site upstream of said recognition region, and a stabilizing region upstream of said nicking enzyme site (see, e.g., U.S. Pat. Nos. 9,689,031; 9,617,586; 9,562,264; and 9,562,263, each of which is incorporated herein by reference in its entirety). The second amplification oligonucleotide comprises a nucleotide sequence comprising a recognition region at the 3′ end that is complementary to the 3′ end of the target sequence sense strand, a nicking enzyme site upstream of said recognition region, and a stabilizing region upstream of said nicking enzyme site. Two nicking enzymes are provided. One nicking enzyme is capable of nicking at the nicking enzyme site of the first amplification oligonucleotide but incapable of nicking within said target sequence. The other nicking enzyme is capable of nicking at the nicking enzyme site of the second amplification oligonucleotide but incapable of nicking within said target sequence. A DNA polymerase is employed under conditions for amplification which involves multiple cycles of extension of the amplification oligonucleotides thereby producing a double-stranded nicking enzyme site which are nicked by the nicking enzymes to produce the amplification product. For example, see U.S. Pat. Nos. 9,689,031; 9,617,586; 9,562,264; 9,562,263; and 10,851,406 and U.S. patent application Ser. Nos. 15/467,893 and 16/243,829, each of which is incorporated herein by reference in its entirety.

In some embodiments, reactions use only two templates to prime, one or two nicking enzymes, and a polymerase, under isothermal conditions. In exemplary embodiments, the polymerase and the nicking enzyme are thermophilic, and the reaction temperature is significantly above the melting temperature of the hybridized target region. The nicking enzyme nicks only one strand in a double-stranded duplex, so that incorporation of modified nucleotides is not necessary as it is in strand displacement. In some embodiments, the method is able to amplify RNA without a separate reverse transcription step, although conversion of RNA to DNA by reverse transcription may be used if desired.

In some embodiments, the method comprises contacting a target DNA molecule comprising a double-stranded target sequence having a sense strand and an antisense strand, with a forward template and a reverse template, wherein said forward template comprises a nucleic acid sequence comprising a recognition region at the 3′ end that is complementary to the 3′ end of the target sequence antisense strand; a nicking enzyme site upstream of said recognition region, and a stabilizing region upstream of said nicking enzyme site; the reverse template comprises a nucleotide sequence comprising a recognition region at the 3′ end that is complementary to the 3′ end of the target sequence sense strand, a nicking enzyme site upstream of the recognition region, and a stabilizing region upstream of the nicking enzyme site; providing a first nicking enzyme that is capable of nicking at the nicking enzyme site of the forward template, and does not nick within the target sequence; providing a second nicking enzyme that is capable of nicking at the nicking enzyme site of the reverse template and does not nick within the target sequence; and providing a DNA polymerase; under conditions wherein amplification is performed by multiple cycles of the polymerase extending the forward and reverse templates along the target sequence producing a double-stranded nicking enzyme site, and the nicking enzymes nicking at the nicking enzyme sites, producing an amplification product.

In certain embodiments, the DNA polymerase is a thermophilic polymerase. In other examples, the polymerase and said nicking enzymes are stable at temperatures up to 37° C., 42° C., 60° C., 65° C., 70° C., 75° C., 80° C., or 85° C. In certain embodiments, the polymerase is stable up to 60° C. The polymerase may, for example, be selected from the group consisting of Bst (large fragment), 9° N, VentR® (exo-) DNA Polymerase, THERMINATOR, and THERMINATOR II (New England Biolabs).

The nicking enzyme may, for example, nick upstream of the nicking enzyme binding site, or the nicking enzyme may nick downstream of the nicking enzyme binding site. In certain embodiments, the forward and reverse templates comprise nicking enzyme sites recognized by the same nicking enzyme and the first and the second nicking enzyme are the same. The nicking enzyme may, for example, be selected from the group consisting of Nt.BspQI, Nb.BbvCi, Nb.BsmI, Nb.BsrDI, Nb.BtsI, Nt.AlwI, Nt.BbvCI, Nt.BstNBI, Nt.CviPII, Nb.Bpu10I, and Nt.Bpu10I.

In some embodiments, the target sequence comprises from 1 to 5 nucleotides more than the sum of the nucleotides of said forward template recognition region and said reverse template recognition region.

In some embodiments, the forward template is provided at the same concentration as the reverse template. In other examples, the forward template is provided at a ratio to the reverse template at the range of ratios of 1:100 to 100:1.

As embodied herein, the NEAR reaction time can be about 10 minutes to about 30 minutes, or about 8 minutes to about 25 minutes, or about 8 minutes to about 20 minutes, or even about 8 minutes to about 15 minutes from the addition of the reagents sufficient to initiate NEAR amplification. In certain embodiments, the NEAR reaction time is about 1 minute to about 20 minutes, about 5 minutes to about 20 minutes, about 8 minutes to about 20 minutes, about 1 minute to about 10 minutes or about 5 minutes to about 10 minutes from the addition of the reagents sufficient to initiate NEAR amplification.

### 4. Nucleic Acid Detection Aspects

In accordance with the disclosed subject matter, and as embodied herein, the methods and systems of the disclosed subject matter include unique nucleic acid detection aspects useful to detect target nucleic acid from a sample, e.g., the methods and systems of the disclosed subject matter for rapid screening of donor blood can include unique nucleic acid detection aspects useful to detect target nucleic acid from a donor sample. For example, but not limitation, reference is now made to various nucleic acid detection methods and system components contemplated in the methods and systems of the present disclosure.

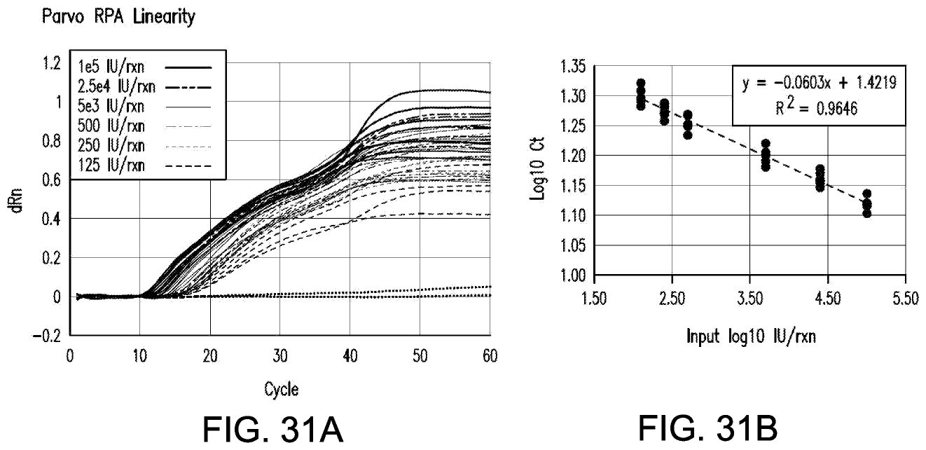

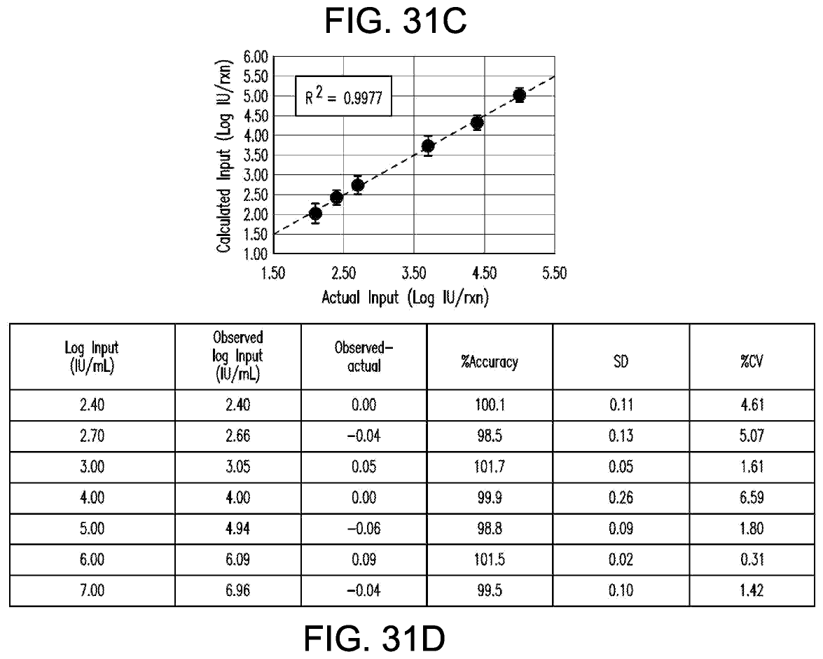

Nucleic acid detection as employed herein is used to determine presence of a nucleic acid or a plurality of different nucleic acids in a sample. Additionally, or alternatively, and in accordance with the disclosed subject matter, e.g., as in FIG. 31, nucleic acid detection is employed to quantify the amount of a nucleic acid or a plurality of different nucleic acids in a sample. As embodied herein, the nucleic acid detection methods and system components can be configured to detect a target nucleic acid or plurality of different nucleic acids using any of a variety or combination of suitable detection techniques.

As described in further detail, detection methods and system components embodied herein can configured to facilitate NAT-based screening. For example, as shown in FIGS. 1 and 2A, detection is performed after or contemporaneously with amplification of one or a plurality of target nucleic acids.

Additionally, or alternatively, as embodied herein, following amplification of one or a plurality of target nucleic acids present in the sample, the methods and system components configured for nucleic acid detection detect such amplified nucleic acid(s) via hybridization. For example, but not limitation, such detection can comprise hybridizing a probe oligonucleotide sufficiently complementary to an amplified target nucleic acid to facilitate detection of the target nucleic acid. In certain embodiments, following hybridization of the probe oligonucleotide to the target nucleic acid, the method comprises detecting hybridization of the probe oligonucleotide to the target nucleic acid. For example, but not limitation, such detection can be achieved by observing a signal from a detectable label, whereby (i) the presence of one or more signals indicates hybridization of the probe oligonucleotide to the target nucleic acid and is indicative of the presence of the pathogen or infectious agent in the sample, and (ii) the absence of a signal indicates the absence of the pathogen or infectious agent in the sample. Detection of a signal from the probe oligonucleotide can be performed using a variety of suitable methodologies, depending on the type of detectable label.

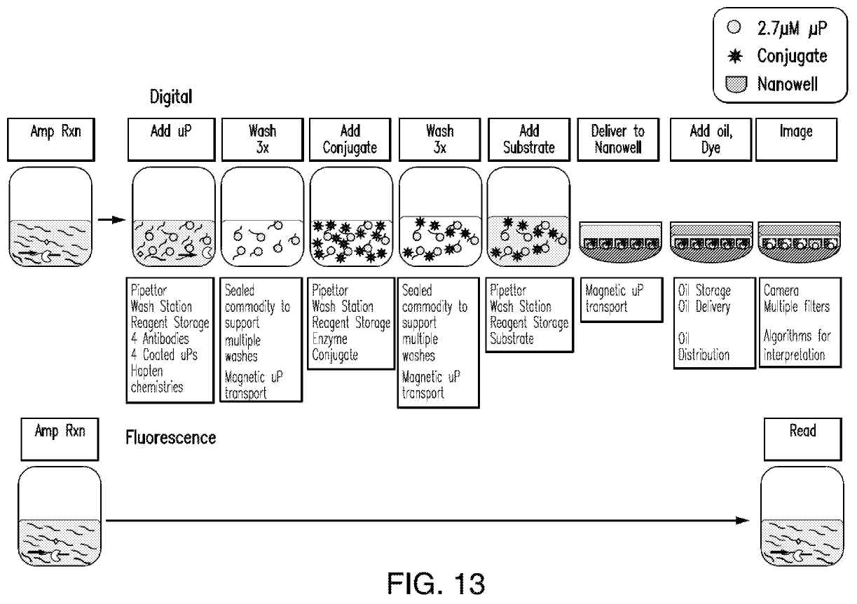

In certain embodiments, the detection operation can employ optical detection, digital detection, and/or other detection methods known in the art. FIG. 13 is a diagram illustrating exemplary digital and optical (e.g., fluorescence-based) target detection strategies. As embodied herein, the detection operation detects the presence or absence of the target nucleic acid.

Additionally, or alternatively, as embodied herein, the methods and systems of the instant disclosure for rapid screening of donor blood can include use of optical detection as disclosed in detail herein.

Additionally, or alternatively, as embodied herein, the methods and systems of the instant disclosure for rapid screening of donor blood can include use of digital detection as disclosed in detail herein.

Additionally, or alternatively, as embodied herein, the methods and systems of the instant disclosure for rapid screening of donor blood can include use of particular target nucleic acid detection aspects allowing for rapid detection of target nucleic acids and which, in certain embodiments, allow for the detection of target nucleic acids at the sensitivity necessary to comply with governmental agency and non-governmental organization regulations and/or guidance for the release of donor blood or a donor material for clinical use.

Additionally, or alternatively, as embodied herein, the methods and systems of the instant disclosure for rapid screening of donor blood can include use of multiplex amplification and/or detection aspects to enhance throughput as disclosed in detail herein.

Additionally, or alternatively, as embodied herein, the methods and systems of the instant disclosure for rapid screening of donor blood can include use of a plurality of detectors capable of detecting the same or different signals as disclosed in detail herein.

Additionally, or alternatively, as embodied herein, the methods and systems of the instant disclosure for rapid screening of donor blood can include use of repeated detection aspects during the amplification strategies as disclosed in detail herein.

Additionally, or alternatively, as embodied herein, the methods and systems of the instant disclosure for rapid screening of donor blood can include use of centrifugal force to move droplets to the bottom of the amplification/detection vessel to enhanced detection as disclosed in detail herein.

In certain embodiments, nucleic acid amplification and nucleic acid detection can occur simultaneously, e.g., in an amplification and detection system. In certain embodiments, an amplification and detection process as disclosed herein includes the simultaneous amplification and detection of nucleic acids in a sample, e.g., an eluate. In certain embodiments, an amplification and detection process of the present disclosure has a duration of about 1 minute to about 60 minutes, about 5 minutes to about 60 minutes, about 8 minutes to about 60 minutes, about 8 minutes to about 50 minutes, about 8 minutes to about 40 minutes, about 8 minutes to about 35 minutes, about 8 minutes to about 30 minutes, about 8 minutes to about 25 minutes, about 8 minutes to about 20 minutes, about 1 minute to about 22 minutes, about 5 minutes to about 22 minutes, about 8 minutes to about 22 minutes, about 1 minute to about 20 minutes, about 5 minutes to about 20 minutes, about 8 minutes to about 20 minutes or about 8 minutes to about 15 minutes. In certain embodiments, an amplification and detection process of the present disclosure has a duration of about 1 minute. In certain embodiments, an amplification and detection process of the present disclosure has a duration of about 5 minutes. In certain embodiments, an amplification and detection process of the present disclosure has a duration of about 20 minutes. In certain embodiments, the amplification and detection process begins with the incubation of an eluate with the reagents sufficient to initiate amplification of a target nucleic acid in the sample, if present, and ends with the determination of a result in the sample, e.g., the detection of the target nucleic acid in the eluate (e.g., the detection of a level in the sample that is greater than a pre-determined level or the detection of a level in the sample that is lower than a pre-determined level) or the lack of detection of the target nucleic acid in the sample.

### 4.1 Optical Detection

As previously noted, and in accordance with an aspect of the disclosed subject matter, the detection operation can employ optical (e.g., fluorescent) detection methods. In certain embodiments, the detection of the amplificated target nucleic acid is mediated by the binding of a labeled probe or by incorporation of a label into amplified copies of the target nucleic acid.

“Label” or “detectable label” as used interchangeably herein refers to a moiety attached to a prop or analyte to render the reaction between the probe and the analyte, or the analyte itself (when the analyte is labeled) detectable, and the probe or analyte so labeled is referred to as “detectably labeled.” A label can produce a signal that is detectable by visual or instrumental means. Various labels include: (i) a tag attached to a specific binding member or analyte by a cleavable linker; or (ii) signal-producing substance, such as chromagens, fluorescent compounds, enzymes, chemiluminescent compounds, radioactive compounds, and the like. Representative examples of labels include moieties that produce light, e.g., acridinium compounds, and moieties that produce fluorescence, e.g., fluorescein. Other labels are described herein. In this regard, the moiety, itself, cannot be detectable but can become detectable upon reaction with yet another moiety. Use of the term “detectably labeled” is intended to encompass such labeling.

Additionally, or alternatively, and in accordance with another aspect of the disclosed subject matter, detection is mediated by observation of a fluorescent label (such as fluorescein (e.g., 5-fluorescein, 6-carboxyfluorescein (e.g., FAM), 3′6-carboxyfluorescein, 5(6)-carboxyfluorescein, 6-hexachloro-fluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), Fluor Orange 560 fluorophore, Quasar 670 fluorophore and Quasar 705 fluorophore. An introduction to labels, labeling procedures and detection of labels is found in Polak and Van Noorden, Introduction to Immunocytochemistry, 2nd ed., Springer Verlag, N.Y. (1997), and in Haugland, Handbook of Fluorescent Probes and Research Chemicals (1996), which is a combined handbook and catalogue published by Molecular Probes, Inc., Eugene, Oreg. A fluorescent label can be used in FPIA (see, e.g., U.S. Pat. Nos. 5,593,896, 5,573,904, 5,496,925, 5,359,093, and 5,352,803, which are hereby incorporated by reference in their entireties)

Additionally, or alternatively, and in accordance with another aspect of the disclosed subject matter, optical detection is performed using fluorescence, chemiluminescence, or other means of generating a signal in response to the presence of an analyte. Many assays are performed by measuring the intensity of a light signal generated in the total volume of a reaction mixture. The light signal generated can be measured by an optical means, wherein the light signal generated is emitted by a large number of molecules. Typically, as described herein, assays can involve combining a sample suspected of containing a target nucleic acid, e.g., target nucleic acids amplified as described herein, with a reagent comprising a labeled probe capable of hybridizing with the target nucleic acid to form a reaction mixture. The signal attributable to the label is then measured after unbound probe is removed from the reaction mixture, typically by performing a wash step. In certain embodiments, the presence of a detectable signal is sufficient to confirm the presence of the target nucleic acid in the sample. In certain embodiments, the signal that is derived from the total volume of the reaction mixture is measured and then compared to a calibration curve to establish the concentration of target nucleic acid present in the sample.

Additionally, or alternatively, and in accordance with another aspect of the disclosed subject matter, the optical detection strategy comprises the use of probes labeled with both a detectable label and a “quencher molecule” where the quencher molecule is capable of interacting with a detectable label to reduce or eliminate the signal emitted by the detectable label. For example, but not by way of limitation, a detection probe employed in the methods and systems of the present disclosure can have a fluorescent moiety that is covalently linked, e.g., to the 5′ end of the probe, and has a quencher molecule, e.g., at the 3′ end of the probe. In the absence of target sequences, the probe adopts a conformation that brings the quencher close enough to the excited fluorophore to absorb its energy before it can be fluorescently emitted. When the probe binds to its complementary sequence in the target, the fluorophore and the quencher are positioned at a sufficient distance apart to allow fluorescent emission and detection. In certain embodiments, the quencher can be selected from any suitable quencher known in the art, such as, for example, BLACK HOLE QUENCHER® 1 (BHQ-1®), BLACK HOLE QUENCHER® 2 (BHQ-2®), BLACK HOLE QUENCHER®-1-dT (BHQ-1 dT®), BLACK HOLE QUENCHER®-2-dT (BHQ-2dT®), IOWA BLACK® FQ, and IO WA BLACK® RQ. For example, but not by way of limitation, an oligonucleotide probe used in the methods and systems of the present disclosure can comprise a FAM fluorophore and a BHQ-1 dT® quencher or a BHQ-2dT® quencher. In certain embodiments, an oligonucleotide probe used in the methods and systems of the present disclosure can include a Quasar 670 fluorophore and a BHQ-1® quencher or a BHQ-2® quencher. In certain embodiments, an oligonucleotide probe used in the methods and systems of the present disclosure can include a Quasar 670 fluorophore and a BHQ-1 dT® quencher or a BHQ-2dT® quencher.

Additionally, or alternatively, and in accordance with another aspect of the disclosed subject matter, specific probes, e.g., probes for specific target nucleic acids and/or internal controls, are each labeled with a different fluorophore, thus allowing for simultaneous detection of a plurality of amplified products.

Additionally, or alternatively, and in accordance with another aspect of the disclosed subject matter, light intensity can be measured using light emitting diodes (LEDs) and/or lasers for excitation and any suitable detector for detection emissions. Fluorescence-optical detection “scanners” can be used which scan the surface of the chip using a focused laser beam, allowing for detection of the emitted fluorescence light. Exemplary fluorescence scanners are described in, e.g., U.S. Pat. Nos. 5,837,475 and 5,945,679. Scanners in which a confocal excitation and detection system has been integrated into an epifluorescence microscope are also known. The systems used in scanners for detecting the emitted fluorescence light are usually “one-channel systems”, i.e., for example, individual photocells or secondary electron multipliers (photomultipliers). Two-dimensional detection systems such as, for example, charged-coupled device (CCD) cameras, also are used for detecting fluorescence or chemiluminescent light of a sample. Commercially available systems have either an optical imaging system which projects the binding surface provided with chemiluminescent markers or fluorescent markers on a CCD sensor by using lens optics, or a combination of image intensifier and CCD camera.

### 4.2 Digital Detection

In accordance with another aspect of the disclosed subject matter, the detection operation can employ digital detection methods. Because every single target nucleic acid, as an end-point entity, can be detected in principle in the context of digital detection, the components and methods associated with digital detection can significantly increase detection sensitivity for sample analysis compared to systems using analog optical detection. As such, digital detection can be performed using a lower concentration of analyte, e.g., target nucleic acids, which can allow for decreased time to process the sample for detection. Additionally, or alternatively, detection can be performed using a smaller sample volume, less reagent material, less conjugate material, fewer microparticles, or any combination of these, which can reduce costs to perform each assay. As such, and as described herein, sample preparation time can be improved due at least in part to less sample manipulation involved (e.g., faster washing times) and/or improved kinetics of reactions achieved using a lower sample volume, less reagent or conjugate material, and/or fewer particles or beads to obtain an analyte concentration suitable for detection. Assays using less sample volume and/or reagent material can be performed using smaller equipment, which can reduce the footprint of the laboratory system for performing the assays as discussed further herein. In addition, or as a further alternative, increased detection sensitivity can provide additional benefits when used with multiplexing. For example, and without limitation, when multiple analytes and corresponding signals are combined into a single, multiplexed assay, a noise level associated with the detection of each analyte signal can be multiplied to obtain a total noise level of the multiplexed system. By increasing the detection sensitivity of each signal being detected, the improved sensitivity can be multiplied to further reduce the total noise level of the multiplexed system.

Digital detection can provide increased sensitivity due at least in part to a reduction of noise during detection relative to the signal being measured, for example, producing a higher signal-to-noise ratio. Such improved signal-to-noise ratios are possible by coupling the analyte of interest, e.g., a particular target nucleic acid, to an independently detectable end-point entity. For example, but not limitation, and as embodied in the strategy depicted in FIG. 13, amplified target nucleic acids can be immobilized to microparticles and labeled with detectable conjugates, where the conjugate is a detectable end-point entity in that it can emit an independently detectable signal, either directly or via the conversion of a substrate.

Additionally, or alternatively, and in accordance with another aspect of the disclosed subject matter, the detection operation employs a digital nanowell detection process. Additionally, or alternatively, a support medium, such as, but not limited to, microparticles, beads, or other labels, can be mixed with the sample in order to perform the digital detection process after amplification. In certain embodiments, reagents including antibodies and coated microparticles can be combined.

For example, but not by way of limitation, digital nanowell detection processes incorporating microparticles can employ anti-Digoxin microparticles. In certain embodiments, digital nanowell detection incorporating microparticles can be performed in a formulation comprising: Tris-HCl, NaCl, BSA, Tergitol 15-s-40, Sodium azide and 0.02% anti-Digoxin μP (microparticles). For example, but not by way of limitation, digital nanowell detection incorporating microparticles can be performed in the following context: about 50 mM Tris-HCl at a pH of about 8.0; about 150 mM NaCl; about 0.2% BSA; about 0.5% Tergitol 15-s-40; about 0.08% Sodium azide; and about 0.02% anti-Digoxin μP (microparticles). The solution can be washed, for example to remove excess reagents and/or unbound analyte. Any suitable number of washes can be performed for each washing step, including one, two, or three or more washes, and each wash can be performed in a single chamber or location or among different chambers or locations. For example, and not limitation, as embodied herein, three washes can be performed.

Additionally, or alternatively, and in accordance with another aspect of the disclosed subject matter, a conjugate can be added to bind with an analyte of interest in the sample. In certain embodiments, a conjugate, e.g., Alkaline Phosphatase-SA, can be added to the sample. In certain embodiments, additional reagents including, but not limited to, Tris-HCl, NaCl, MgCl₂, ZnCl₂, fish gelatin, Rabbit IgG, Saponin, calf serum, Goat IgG and Sodium azide, can be added to the sample. For example, and not limitation, the conjugate can include one or more reagents or enzymes selected or configured to react with the analyte of interest to produce a signal for detection by the detection component. In certain embodiments, the digital nanowell detection process will employ conjugates in the following context: about 3000 pM Alkaline Phosphatase-SA; about 100 mM Tris-HCl, at a pH of about 7.5; about 500 mM NaCl; about 1 mM MgCl₂; about 0.1 ZnCl₂; about 8.9 g/L fish gelatin; about 30 ug/mL of Rabbit IgG; about 0.1% Saponin; about 10% calf serum; about 5 mg/mL Goat IgG; and about 0.1% Sodium azide. The solution can be washed, for example to remove excess conjugate unbound to the analyte of interest. Any suitable number of washes can be performed for each washing step, including one, two, or three or more washes, and each wash can be performed in a single chamber or location or among different chambers or locations.

Additionally, or alternatively, and in accordance with another aspect of the disclosed subject matter, detection of the microparticles bound with analytes and conjugates can be performed in a single chamber or location or among different chambers or locations. For purpose of illustration and not limitation, the detection chamber or location can include a surface and a detection region. The microparticles can be added to the detection chamber or location using any suitable technique, including but not limited to pipetting, magnetic force or dielectrophoresis. In certain embodiments, the digital nanowell detection process will employ a detection substrate, e.g., AJ Phos. In certain embodiments, additional reagents including, but not limited to, DEA, MgCl₂ and Tween 20, can be added in combination with the detection substrate. For example, but not by way of limitation, the digital nanowell detection process will employ a detection substrate in the following context: about 200 μM AJ Phos; about 1 M DEA; about 1 mM MgCl₂; and about 0.05% Tween 20. As embodied herein, the detection region can include one or more nanowells. The microparticles can be moved to the detection region, for example and as embodied herein, an array of nanowells. The microparticles can be moved to the nanowells using any suitable technique, including but not limited to pipetting, magnetic force or dielectrophoresis. In certain embodiments, oil, e.g., 3 mM Guaiazulene in FC-40 oil, is added to seal the nanowells. In certain embodiments, a dye can be added to increase contrast or otherwise improve optical conditions for detection of the analyte of interest in the nanowells. In certain embodiments, the digital nanowell detection process incorporating microparticles will employ a dye in the following context: about 0.1% Tween 20; about 10 mM PBS; and about 50 mM Nigrosine. In certain embodiments, one or more images of the microparticles are taken and analyzed to determine the presence or absence of the analyte of interest and/or a concentration of the analyte of interest in the sample.

### 4.3 NAT-Based Screening for Specific Target Nucleic Acids

In accordance with another aspect of the disclosed subject matter, the methods and systems of the present disclosure can be employed in performing NAT-based screening to detect a specific set of target nucleic acids associated with one or more of a plurality of pathogens or infectious agents. For example, but not limitation, the set of pathogens or infectious agents can be a set identified by a governmental agency or non-governmental organization. Additionally, or alternatively, the set of pathogens or infectious agents are those identified by a governmental agency or non-governmental organization as necessary to ensure the safe release of the donor blood. For example, but not by way of limitation, the set of pathogens or infectious agents can be those identified by the U.S. Food and Drug Agency as required to ensure the safe release of donor blood. In certain embodiments, the set of pathogens or infectious agents can be those identified in 21 CFR § 1271 and the related codes cited therein, all of which are incorporated by reference herein in their entirety. Additionally, or alternatively, the set of pathogens or infectious agents can be those identified by the World Health Organization in “Screening Donated Blood for Transfusion-Transmissible Infections: Recommendations” World Health Organization (2009), which is incorporated by reference herein in its entirety.

Additionally, or alternatively, the target nucleic acid is a bacterial, eukaryotic (e.g., eukaryotic parasite), or viral nucleic acid. In certain embodiments, the target nucleic acid is a nucleic acid derived from SARS-CoV-2 (COVID-19), coronaviruses, HIV (e.g. HIV-1 and/or HIV-2), Hepatitis B (HBV), Hepatitis C (HCV), Hepatitis A (HAV), Hepatitis E (HEV), Cytomegalovirus (CMV), Parvovirus B19, Creutzfeldt-Jakob disease (vCJD), Chlamydia, Gonorrhea, West Nile virus (WNV), Zika virus (ZIKV), Dengue, Chikungunya, Influenza (e.g., Influenza A virus, Influenza B virus, or Influenza C virus), Babesia, Malaria, Rubella, Varicella-zoster, Herpes Simplex, Polio, syphilis, Smallpox, Vaccinia, Rabies, human T-lymphotropic virus (HTLV), Usutu Virus or Epstein Barr Virus. In certain embodiments, the target nucleic acid is a nucleic acid derived from one or more new or emerging pathogens, viruses and/or agents.

Additionally, or alternatively, the selection of a specific target nucleic acid depends on the type of nucleic acid that comprises the genome of the virus. For example, but not by way of limitation, if the virus is an RNA-based virus, e.g., HIV-1 and HCV, the nucleic acids to be detected will be RNA. If the virus is a DNA-based virus, e.g., HBV, the nucleic acids to be detected will be DNA. In certain embodiments, the methods can detect ribosomal RNA of the parasite Babesia.

Additionally, or alternatively, the methods and systems of the present disclosure find particular use in the context of donor blood screening due to the methods' and systems' sensitivity. “Sensitivity,” as used herein, refers to a method or system's capability to detect a predetermined level of one or more of a plurality of pathogens or infectious agents. As illustrated in Example 23, the methods and systems of the present disclosure are capable of detecting the predetermined level of one or more of a plurality of pathogens or infectious agents wherein those predetermined levels are equivalent to or lower than the predetermined levels achieved by current NAT-based screening methods.

Additionally, or alternatively, the methods and system components for nucleic acid detection can screen for the presence or absence of each of a menu of bacterial, eukaryotic (e.g., eukaryotic parasite), and/or viral nucleic acids. In certain embodiments, the methods and system components for nucleic acid detection can screen for a predetermined level of each of a menu of bacterial, eukaryotic (e.g., eukaryotic parasite), and/or viral nucleic acids. Additionally, or alternatively, the menu of bacterial, eukaryotic (e.g., eukaryotic parasite), or viral nucleic acid selected for screening can be screened using one or more samples. For example, in certain embodiments, one sample can be used to screen for a first subset of the menu and one or more (e.g., a second, third, fourth, or fifth) additional samples are screened for the remaining members of the selected menu. In certain embodiments, the methods and systems of the present disclosure can screen for a predetermined level of each of HIV-1, HIV-2, HCV, and HBV. Additionally, or alternatively, the methods and systems of the present disclosure can screen for a predetermined level of each of HIV-1, HIV-2, HCV, HBV, and WNV. Additionally, or alternatively, the methods and systems of the present disclosure can screen for a predetermined level of each of HIV-1, HIV-2, HCV, HBV, WNV, and Zika Virus. Additionally, or alternatively, the methods and systems of the present disclosure can screen for a predetermined level of each of HIV-1, HIV-2, HCV, HBV, WNV, Zika Virus, and Chikungunya Virus. Additionally, or alternatively, the methods and systems of the present disclosure can screen for a predetermined level of each of HIV-1, HIV-2, HCV, HBV, WNV, Zika Virus, Chikungunya Virus, and Dengue Virus. Additionally, or alternatively, the methods and systems of the present disclosure can screen for a predetermined level of each of HIV-1, HIV-2, HCV, HBV, WNV, Zika Virus, Chikungunya Virus, Dengue Virus, and Babesia. Additionally, or alternatively, the methods and systems of the present disclosure can screen for a predetermined level of each of HIV-1, HIV-2, HCV, HBV, WNV, Zika Virus, Chikungunya Virus, Dengue Virus, Babesia, and Malaria. Additionally, or alternatively, the methods and systems of the present disclosure can screen for a predetermined level of each of HIV-1, HIV-2, HCV, HBV, WNV, Zika Virus, Chikungunya Virus, Dengue Virus, Babesia, Malaria, and Parvovirus B19. Additionally, or alternatively, the methods and systems of the present disclosure can screen for a predetermined level of each of HIV-1, HIV-2, HCV, HBV, WNV, Zika Virus, Chikungunya Virus, Dengue Virus, Babesia, Malaria, Parvovirus B19, and HAV. Additionally, or alternatively, the methods and systems of the present disclosure can screen for a predetermined level of each of HIV-1, HIV-2, HCV, HBV, WNV, Zika Virus, Chikungunya Virus, Dengue Virus, Babesia, Malaria, Parvovirus B19, HAV, and HEV.

Additionally, or alternatively, the methods and systems of the present disclosure can screen for a predetermined level of each of Zika Virus and WNV. Additionally, or alternatively, the methods and systems of the present disclosure can screen for a predetermined level of each of Chikungunya Virus and Dengue Virus. Additionally, or alternatively, the methods and systems of the present disclosure can screen for a predetermined level of each of Zika Virus, WNV, Chikungunya Virus and Dengue Virus. Additionally, or alternatively, the methods and systems of the present disclosure can screen for a predetermined level of each of HIV-1, HIV-2, HCV, HBV, and Zika Virus. Additionally, or alternatively, the methods and systems of the present disclosure can screen for a predetermined level of each of HIV-1, HIV-2, HCV, HBV, Zika Virus, and Dengue Virus. Additionally, or alternatively, the methods and systems of the present disclosure can screen for a predetermined level of each of HIV-1, HIV-2, HCV, HBV, Zika Virus, Dengue Virus, and Chikungunya Virus. Additionally, or alternatively, the methods and systems of the present disclosure can screen for a predetermined level of each of HIV-1, HIV-2, HCV, HBV, Zika Virus, Dengue Virus, and WNV. Additionally, or alternatively, the methods and systems of the present disclosure can screen for a predetermined level of each of HIV-1, HIV-2, HCV, HBV, Zika Virus, WNV, and Chikungunya Virus. Additionally, or alternatively, the methods and systems of the present disclosure can screen for a predetermined level of each of HIV-1, HIV-2, HCV, HBV, Zika Virus, WNV, Dengue Virus, and Chikungunya Virus. Additionally, or alternatively, the methods and systems of the present disclosure can screen for a predetermined level of each of HIV-1, HIV-2, HCV, HBV, and Dengue Virus. Additionally, or alternatively, the methods and systems of the present disclosure can screen for a predetermined level of each of HIV-1, HIV-2, HCV, HBV, Dengue Virus, and Chikungunya Virus. Additionally, or alternatively, the methods and systems of the present disclosure can screen for a predetermined level of each of HIV-1, HIV-2, HCV, HBV, Dengue Virus, WNV, and Chikungunya Virus. Additionally, or alternatively, the methods and systems of the present disclosure can screen for a predetermined level of each of HIV-1, HIV-2, HCV, HBV, and Chikungunya Virus. Additionally, or alternatively, the methods and systems of the present disclosure can screen for a predetermined level of each of HIV-1, HIV-2, HCV, HBV, Chikungunya Virus, and Zika Virus. Additionally, or alternatively, the methods and systems of the present disclosure can screen for a predetermined level of each of HIV-1, HIV-2, HCV, HBV, Chikungunya Virus, Zika Virus, and WNV. Additionally, or alternatively, the methods and systems of the present disclosure can screen for a predetermined level of each of HIV-1, HIV-2, HCV, HBV, WNV, and Dengue Virus. Additionally, or alternatively, the methods and systems of the present disclosure can screen for a predetermined level of each of HIV-1, HIV-2, HCV, HBV, Chikungunya Virus, Zika Virus, and WNV.

Additionally, or alternatively, the methods and systems of the present disclosure can screen for a predetermined level of each of Babesia and Malaria. Additionally, or alternatively, the methods and systems of the present disclosure can screen for a predetermined level of each of HIV-1, HIV-2, HCV, HBV, and Babesia. Additionally, or alternatively, the methods and systems of the present disclosure can screen for a predetermined level of each of HIV-1, HIV-2, HCV, HBV, and Malaria. Additionally, or alternatively, the methods and systems of the present disclosure can screen for a predetermined level of each of HIV-1, HIV-2, HCV, HBV, Malaria, and Babesia.

Additionally, or alternatively, the methods and systems of the present disclosure can screen for a predetermined level of each of Parvovirus B19 and HAV Additionally, or alternatively, the methods and systems of the present disclosure can screen for a predetermined level of each of HIV-1, HIV-2, HCV, HBV, and Parvovirus B19. Additionally, or alternatively, the plurality of pathogens or infectious agents are HIV-1, HIV-2, HCV, HBV, Parvovirus B19, and HAV.

Additionally, or alternatively, the methods and systems of the present disclosure can screen for a predetermined level of each of HIV-1, HIV-2, HCV, HBV, and HAV.

Additionally, or alternatively, the methods and systems of the present disclosure can screen for a predetermined level of each of HIV-1, HIV-2, HCV, HBV, and HEV.

### 4.4 Sensitivity of NAT-Based Screening for Specific Target Nucleic Acids

As the methods and systems of the present disclosure can be employed in performing NAT-based screening of donor blood, among other important pathogen and infectious agent detection efforts, the methods and systems of the disclosed subject matter can be configured with the sensitivity necessary to comply with governmental agency and non-governmental organization regulations and/or guidance. For example, but not by way of limitation, the sensitivity, i.e., “limit of detection,” of the methods and systems described herein for screening of pathogens or infectious agents can be identified by a governmental agency or non-governmental organization. For example, but not limitation, the limit of detection of pathogens or infectious agents are those identified by a governmental agency or non-governmental organization as necessary to ensure the safe release of donor blood.

Although specific target amplicon regions, specific primer sequences, and specific probe sequences are referenced herein, such sequences are provided as non-limiting exemplary regions and/or sequences. As would be appreciated by one of skill in the art, a variety of suitable target amplicon regions for any particular pathogen or infectious agent can be employed alone or in combination within the context of the methods and systems of the present disclosure. Similarly, the present disclosure is not limited to the expressly recited primer and probe sequences, as one of skill in the art would appreciate that not only would variants of the disclosed sequences, e.g., primer and probe sequences targeting sequences that overlap with one or more nucleotide targeted by an expressly recited primer or probe, as well as certain entirely distinct primer and probe sequences, e.g., those targeting sequences that do not overlap with the expressly recited primer and probe sequences, would also be suitable for use in the methods and systems of the present disclosure.

In certain embodiments, the primer sequences and probe sequences disclosed herein can be modified to increase the sensitivity of a nucleic acid analysis of the present disclosure. For example, but not by way of limitation, one or more modifications can be introduced into the nucleotide sequences of the disclosed primer sequences and/or probe sequences to increase sensitivity, e.g., one or more, two or more, three or more, four or more, five or more modifications can be introduced into the nucleotide sequences of the disclosed primer sequences and/or probe sequences to increase sensitivity. In certain embodiments, the primer sequences and probe sequences disclosed herein can be modified to introduce one or more mismatches to the sequence of the complementary target nucleic acid, e.g., one or more, two or more, three or more, four or more or five or more mismatches to the sequence of the complementary target nucleic acid. In certain embodiments, a mismatch can be introduced towards the 3′ end of a primer or probe disclosed herein. In certain embodiments, a mismatch can be introduced towards the 5′ end of a primer or probe disclosed herein. In certain embodiments, the introduction of a mismatch in one or more primers, e.g., a forward primer and/or a reverse primer, of the present disclosure can reduce the interaction of the primer with a probe used in the same isothermal amplification reaction. In certain embodiments, the primer sequences and/or probe sequences disclosed herein can be modified to introduce one or more non-canonical nucleotides, e.g., substituting one or more nucleotides of the presently disclosed primer sequences and/or probe sequences with a non-canonical nucleoside. Non-limiting examples of non-canonical nucleosides include inosine, xanthine hypoxanthine, isocytosine and isoguanine. In certain embodiments, the primer sequences and/or probe sequences disclosed herein can be modified to introduce one or more locked nucleic acids (LNAs), e.g., substitution of one or more nucleotides of the presently disclosed primer sequences and/or probe sequences with an LNA. In certain embodiments, a probe disclosed herein can be modified by modifying the linkage of the fluorophore and/or quencher to the probe to improve sensitivity.

In certain embodiments, a primer or probe for use in the present disclosure can have a nucleotide sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% greater identity of an oligonucleotide disclosed herein. In certain embodiments, a primer or probe for use in the present disclosure can have a nucleotide sequence that is about 95% greater identity of an oligonucleotide disclosed herein. In certain embodiments, a primer or probe for use in the present disclosure can have a nucleotide sequence that is about 99% greater identity of an oligonucleotide disclosed herein. In certain embodiments, a primer or probe for use in the present disclosure can include a nucleotide sequence that is identical to or differs by no more than 5 nucleotides, e.g., differs by no more than 4 nucleotides, differs by no more than 3 nucleotides, differs by no more than 2 nucleotides or differs by no more than 1 nucleotide, from a nucleotide sequence disclosed herein.

Additionally, or alternatively, the methods and systems for sample analysis described herein are sufficiently sensitive to achieve a limit of detection of 1-100 copies/mL for the detection of HIV-1. In certain embodiments, the methods and systems are sufficiently sensitive to achieve a limit of detection of 1-50 copies/mL for the detection of HIV-1. In certain embodiments, the methods and systems are sufficiently sensitive to achieve a limit of detection of 1-25 copies/mL for the detection of HIV-1. In certain embodiments, the methods and systems are sufficiently sensitive to achieve a limit of detection of 20 copies/mL for the detection of HIV-1.

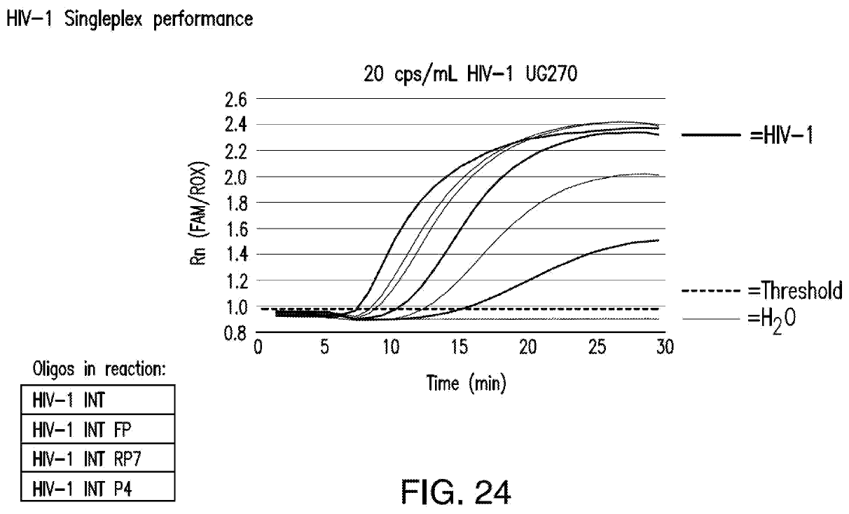

The HIV-1 genomic organization consists of a core region that includes the gag, pol, and env gene regions flanked by long terminal repeats (LTR) on either end. In certain embodiments, the HIV-1 RPA design consists of one target amplicon region. In certain embodiments, the HIV-1 RPA design consists of two or more target amplicon regions. In certain embodiments, the amplification/detection target is in the INT gene region, located within the pol gene (where “FP” is forward primer; “RP” is reverse primer; and “P” is probe). In certain embodiments, the amplification/detection target is within the 5′ LTR region. In certain embodiments, the target of amplification/detection is within the gag gene. Non-limiting examples of primers and probes for detecting HIV-1 are provided below and in Table 14, Table 15, Table 18, FIG. 24, Example 13 and Example 24.

Target (int): 176 bp amplicon

| Oligo ID | Sequence |
|---|---|
| HIV-1 INT FP1 | 5′-ATTCCCTACAATCCCC |
| | AAAGTCAAGGAGT-3′ |
| HIV-1 INT RP7 | 5′-TCTTTCCCCTGCACTG |
| | TACCCCCCAAT-3′ |
| HIV-1 INT P4 | 5′-ACAGCAGTACAAATGGC |
| | AGTATTCATTCACAA[T(FAM)] |
| | [dSpacer]T[T(BHQ-1)]AAA |
| | AGAAAAGGGG-SpacerC3-3′ |

HIV-1 target within the 5′ LTR region:
191 bp amplicon

| | |
|---|---|
| HIV-1 LTR FP1 | 5′-AAGCCTCAATAAAGC |
| | TTGCCTTGAGTGC-3′ |
| HIV-1 LTR RP3 | 5′-CTTCAGCAAGCCGAG |
| | TCCTGCGTCGAGAG-3′ |
| HIV-1 LTR P1 | 5′-GTGTGTGCCCGTCTG |
| | TTGTGTGACTCTGG[T |
| | (FAM)]A[dSpacer]C |
| | [T(BHQ-1)]AGAGATC |
| | CCTCAG-SpacerC3-3′ |

Additionally, or alternatively, the methods and system components for nucleic acid detection described herein are sufficiently sensitive to achieve a limit of detection of 1-40 IU/mL for HIV-2. In certain embodiments, the methods and system components are sufficiently sensitive to achieve a limit of detection of 1-20 IU/mL for the detection of HIV-2. In certain embodiments, the methods and system components are sufficiently sensitive to achieve a limit of detection of 1-15 IU/mL for the detection of HIV-2. In certain embodiments, the methods and system components are sufficiently sensitive to achieve a limit of detection of 10 IU/mL for the detection of HIV-2.

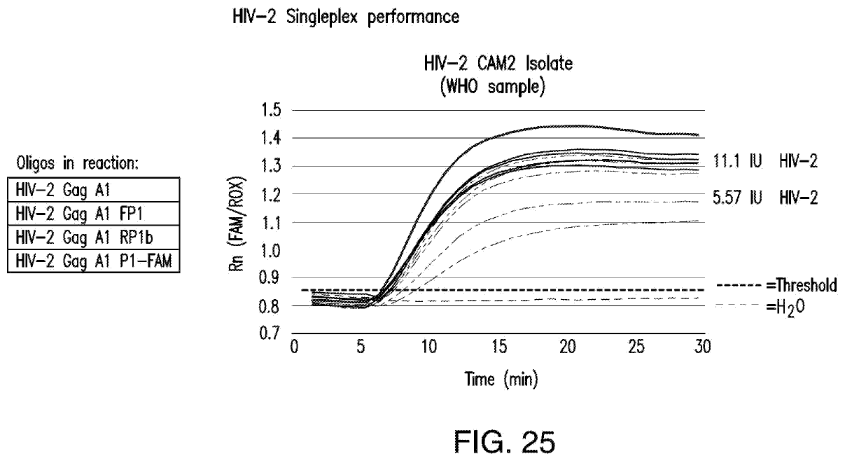

The HIV-2 genomic organization closely mimics that of HIV-1; there is a core region of the gag, pol, and env genes flanked by long terminal repeats (LTR) on either end. In certain embodiments, the target of amplification/detection is the pol gene. In certain embodiments, the target of amplification/detection is the gag gene and/or the 5′ LTR region. Non-limiting examples of primers and probes for detecting HIV-2 are provided below and in Table 14, Table 15, Table 18, FIG. 25, Example 13 and Example 24.

Target pol: 106 bp amplicon

HIV-2 Pol FP3
HIV-2 Pol RP6
HIV-2 Pol P1

Target gag:

HIV-2 Gag FP8
HIV-2 Gag RP1b
HIV2 Gag P1-D5

Target LTR

HIV-2 LTR A3 FP4
HIV-2 LTR A3 RP1
HIV-2 LTR A3 P2

Target pol (second target):

HIV-2 Pol FP2
HIV-2 Pol RP8
HIV-2 Pol P2

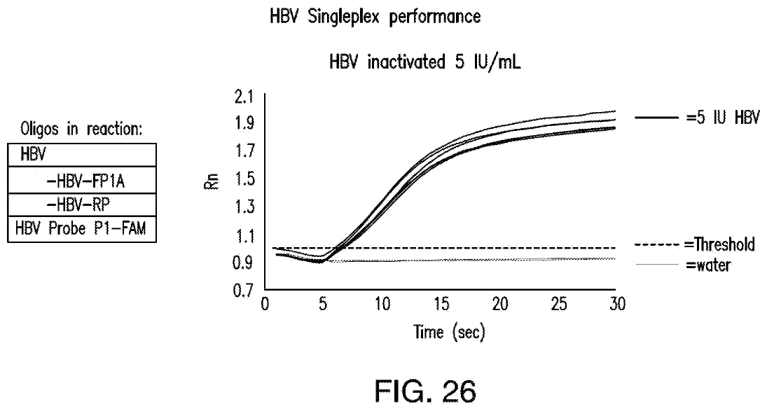

Additionally, or alternatively, the methods and system components for nucleic acid detection described herein are sufficiently sensitive to achieve a limit of detection of 1-20 IU/mL, for the detection of HBV. In certain embodiments, the methods and system components are sufficiently sensitive to achieve a limit of detection of 1-15 IU/mL for the detection of HBV. In certain embodiments, the methods and system components are sufficiently sensitive to achieve a limit of detection of 1-10 IU/mL for the detection of HBV. In certain embodiments, the methods and system components are sufficiently sensitive to achieve a limit of detection of 5 IU/mL for the detection of HBV. In certain embodiments, the target of amplification/detection is in the HbS gene. Non-limiting examples of primers and probes for detecting HBV are provided below and in Table 14, Table 15, Table 18, FIG. 26, Example 13 and Example 24.

HBV target (HbS gene): 136 bp amplicon

| | |
|---|---|
| HBV-FP1a-M5 | |
| HBV-RP | |
| HBV-P5 | 5′-AGCGCTGGATGTGTCTGC |
| | GGCGTTTTATCA-T(Quasar 705)- |
| | dSpacer-T-T(BHQ-2)-CCTC |
| | TTCATCCTG-SpacerC3-3′ |

HBV target (second HbS gene target)

| | |
|---|---|
| HBV- | 5′-CTAGACTCGTGGTGGACT |
| FPe | TCTCTCAATTTTCT-3′ |
| HBV RP | |
| HBV P2 | 5′-CTTGGCCAAAATTCGCAGTCCCCAACC |
| | TCCAA[T(FAM)]C[dSpacer]C[T(BHQ- |
| | 1]CACCAACCTCCTGTC-SpacerC3-3′ |

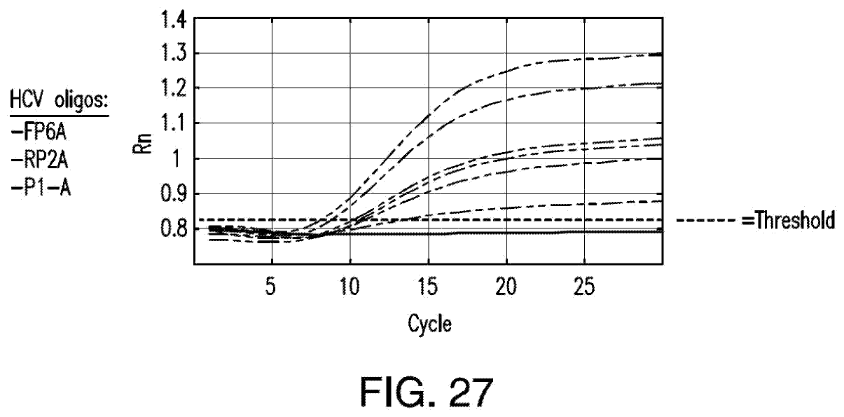

Additionally, or alternatively, the methods and system components for nucleic acid detection described herein are sufficiently sensitive to achieve a limit of detection of 1-100 IU/mL, for the detection of HCV. In certain embodiments, the methods and system components are sufficiently sensitive to achieve a limit of detection of 1-50 IU/mL for the detection of HCV. In certain embodiments, the methods and system components are sufficiently sensitive to achieve a limit of detection of 1-25 IU/mL for the detection of HCV. In certain embodiments, the methods and system components are sufficiently sensitive to achieve a limit of detection of 10 IU/mL for the detection of HCV (where “RC” is the probe). In certain embodiments, the amplification/detection target is within the 5′ UTR region. Non-limiting examples of primers and probes for detecting HCV are provided below and in Table 14, Table 15, Table 18, FIG. 27, Example 13 and Example 24.

HCV target (5′ UTR): 120 bp amplicon

| Oligo ID | Sequence |
|---|---|
| HCV FP6a | 5′-CAATGCCTGGAGATTT |
| | GGGCGTGCCCCCGCAAG-3′ |
| HCV RP2a | |
| HCV RC3 | |

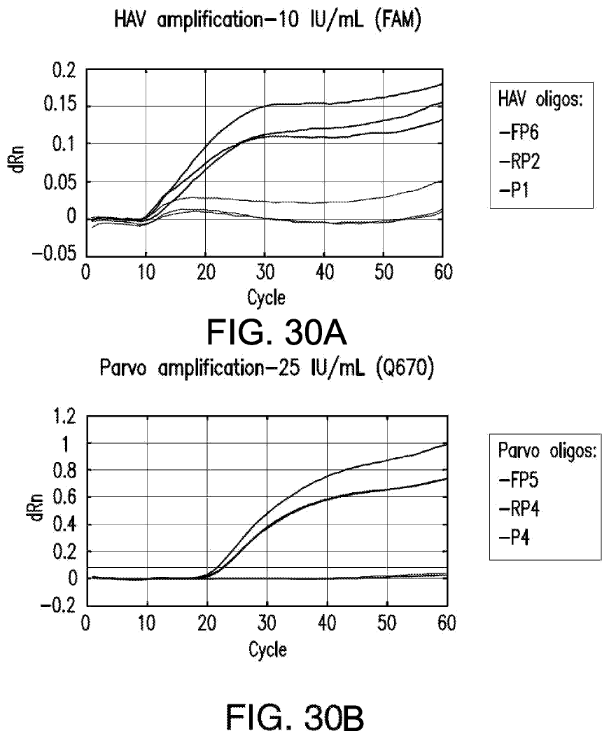

Additionally, or alternatively, the methods and system components for nucleic acid detection described herein are sufficiently sensitive to achieve a limit of detection of 1-80 IU/mL for the detection of Parvovirus B19. In certain embodiments, the methods and system components are sufficiently sensitive to achieve a limit of detection of 1-50 IU/mL for the detection of Parvovirus B19. In certain embodiments, the methods and system components are sufficiently sensitive to achieve a limit of detection of 1-25 IU/mL for the detection of Parvovirus B19. In certain embodiments, the methods and system components are sufficiently sensitive to achieve a limit of detection of 10 IU/mL for the detection of Parvovirus B19. In certain embodiments, the amplification/detection target is within a gene encoding a nucleocapsid protein, e.g., the VP1 gene. In certain embodiments, the amplification/detection target is within the NS1 gene. Non-limiting examples of primers and probes for detecting Parvovirus B19 are provided below and in Table 12, FIG. 30, Example 8, Example 14, Example 15 and Example 23.

Parvovirus B19 target (VP1):

| Oligo ID | Sequence (5′-3′) | 3′ |
|---|---|---|
| VP1-P3 | CAGAACCTAGAGGAGAAG | C3-Spacer |
| | ATGCAGTATTATC-T(Q670)- | |
| | A-dSpacer-T(BHQ-2)- | |
| | GAAGACTTACACAA | |
| VP1-FP5 | TATCTGACCACCCCCA | |
| | TGCCTTATCATCCAGT | |
| VP1-RP4 | TGGCTATACCTAAAGT | |
| | CATGAATCCTTGCAGC | |

Parvovirus B19 FPs within VP1:

| Oligo ID | Sequence (5′-3′) |
|---|---|
| VP1-FP1 | ATTTTCAAAGTCATGGACAGTTATCTGACCAC |
| VP1-FP2 | CAAAGTCATGGACAGTTATCTGACCACCCCCA |
| VP1-FP3 | CCATGGACAGTTATCTGACCACCCCCATGCCT |
| VP1-FP4 | GACAGTTATCTGACCACCCCCATGCCTTATCA |

Parvovirus B19 RPs within VP1:

| Oligo ID | Sequence (5′-3′) |
|---|---|
| VP1-RP1 | GTCATGAATCCTTGCAGCACTGTCAACAGCA |
| VP1-RP2 | TACCTAAAGTCATGAATCCTTGCAGCACTGTC |
| VP1-RP3 | GCTATACCTAAAGTCATGAATCCTTGCAGCAC |

Parvovirus B19 probes within VP1:

| Oligo ID | Sequence (5′-3′) | 3′ |
|---|---|---|
| VP1-P2 | CAGTCATGCAGAACCTAGAGGAGAA | C3-Spacer |
| | GATGCAG-T(FAM)-dSpacer-T- | |
| | T(BHQ-1)-ATCTAGTGAAGAC | |
| VP1-P7 | GGCAAGTTAGCGTACAACTACCCGG | C3-Spacer |
| | TACTAACTA-T(FAM)-dSpacer-T- | |
| | T(BHQ-1)-GGGCCTGGCAATG | |

Parvovirus B19 Target (NS1):

| Oligo ID | Sequence (5′-3′) | 3′ |
|---|---|---|
| NS1-P5 | TGGTGAAAGCTCTGAAGAACTCAGTGAAAGCAGC-T(FAM)-T-dSpacer- | C3-spacer |
| | T(BHQ-1)-TTAACCTCATCACC | |
| NS1-FP6 | AGTGGTGGTGAAAGCTCTGAAGAACTCAGTGA | |
| NS1-RP4 | GAGGAAACTGGGCTTCCGACAAATGATTCTCC | |

FPs within NS1:

| Oligo ID | Sequence (5′-3′) |
|---|---|
| NS1-FP3 | AGTATCAGCAGCAGTGGTGGTGAAAGCTCTGA |
| NS1-FP5 | GCAGCAGTGGTGGTGAAAGCTCTGAAGAACTC |

Parvovirus B19 RPs within NS1:

| Oligo ID | Sequence (5′-3′) |
|---|---|
| NS1-RP6 | CTTAGCCAATTGGCTATACCTAAAGTCATGAA |

Parvovirus B19 probes within NS1:

| Oligo ID | Sequence (5′-3′) | 3′ |
|---|---|---|
| NS1-P3 | TGAAAGCTCTGAAGAACTCAGTGAAAGCAGCTTT-T(FAM)-dSpacer- | C3-Spacer |
| | T(BHQ-1)-AACCTCATCACCCC | |

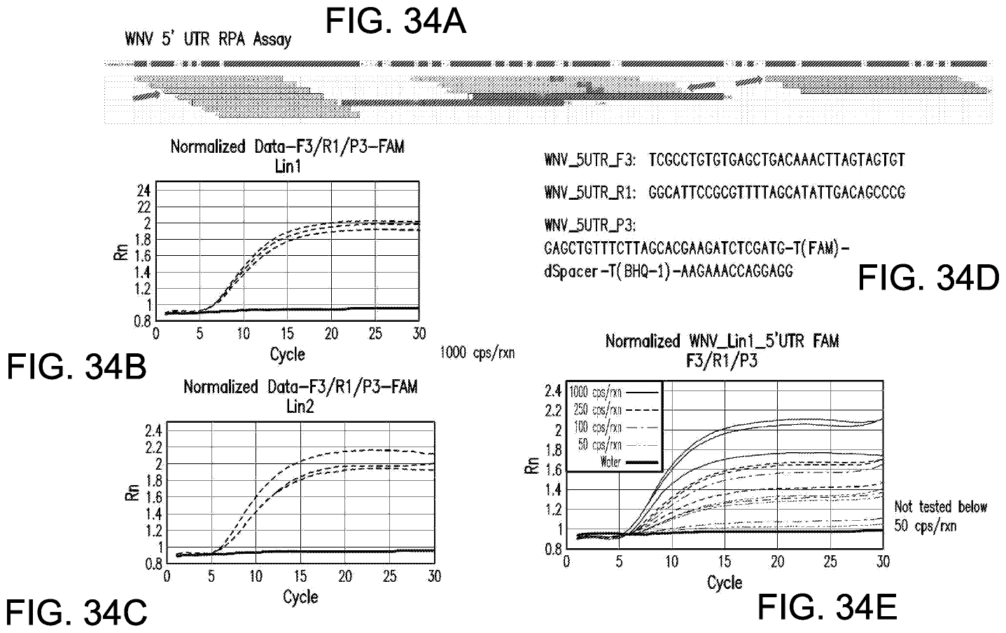

Additionally, or alternatively, the methods and system components for nucleic acid detection described herein are sufficiently sensitive to achieve a limit of detection of 1-100 copies/mL for the detection of WNV. In certain embodiments, the methods and system components are sufficiently sensitive to achieve a limit of detection of 1-50 copies/mL for the detection of WNV. In certain embodiments, the methods and system components are sufficiently sensitive to achieve a limit of detection of 1-25 copies/mL for the detection of WNV. In certain embodiments, the methods and system components are sufficiently sensitive to achieve a limit of detection of 10 copies/mL for the detection of WNV. In certain embodiments, the amplification/detection target is within the 5′ UTR region. Non-limiting examples of primers and probes for detecting WNV are provided below and in FIG. 34.

WNV Primers and Probe

| Oligo ID | Sequence (5′-3′) |
|---|---|
| WNV_5UTR_F3 | TCGCCTGTGTGAGCTGACAAACTTAGTAGTGT |
| WNV_5UTR_R1 | GGCATTCCGCGTTTTAGCATATTGACAGCCCG |
| WNV_5UTR_P3 | GAGCTGTTTCTTAGCACGAAGATCTCGATG-T(FAM)-dSpacer-T(BHQ-1)- |
| | AAGAAACCAGGAGG |

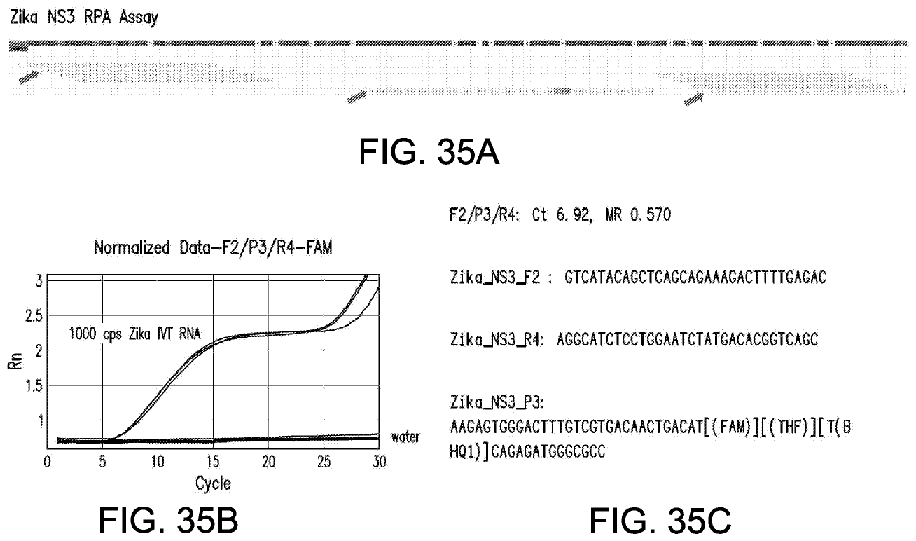

Additionally, or alternatively, the methods and system components for nucleic acid detection described herein are sufficiently sensitive to achieve a limit of detection of 1-100 copies/mL for the detection of Zika. In certain embodiments, the methods and system components are sufficiently sensitive to achieve a limit of detection of 1-50 copies/mL for the detection of Zika. In certain embodiments, the methods and system components are sufficiently sensitive to achieve a limit of detection of 1-25 copies/mL for the detection of Zika. In certain embodiments, the methods and system components are sufficiently sensitive to achieve a limit of detection of 10 copies/mL for the detection of Zika. In certain embodiments, the amplification/detection target is within the NS3 gene. Non-limiting examples of primers and probes for detecting Zika are provided below and in Example 19 and FIG. 35.

Zika Primers and Probe

| Oligo ID | Sequence (5′-3′) |
|---|---|
| Zika_NS3_F2 | GTCATACAGCTCAGCAGAAAGACTTTTGAGAC |
| Zika_NS3_R4 | AGGCATCTCCTGGAATCTATGACACGGTCAGC |
| Zika_NS3_P3 | AAGAGTGGGACTTTGTCGTGACAACTGACAT[(FAM)][(THF)] |
| | [T(BHQ1)]CAGAGATGGGCGCC |

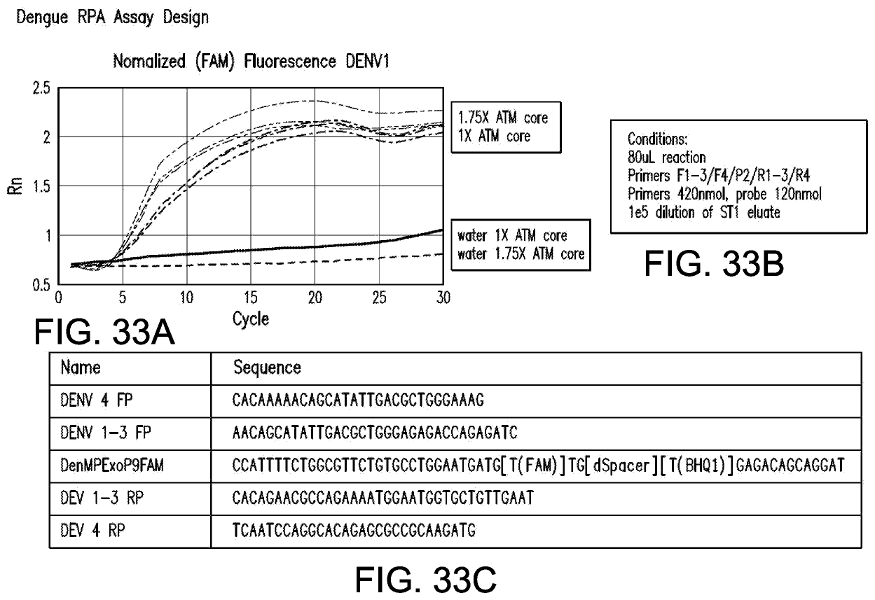

Additionally, or alternatively, the methods and system components for nucleic acid detection described herein are sufficiently sensitive to achieve a limit of detection of 1-100 copies/mL for the detection of Dengue. In certain embodiments, the methods and system components are sufficiently sensitive to achieve a limit of detection of 1-50 copies/mL for the detection of Dengue. In certain embodiments, the methods and system components are sufficiently sensitive to achieve a limit of detection of 1-25 copies/mL for the detection of Dengue. In certain embodiments, the methods and system components are sufficiently sensitive to achieve a limit of detection of 10 copies/mL for the detection of Dengue. In certain embodiments, the amplification/detection target is within the 3′ UTR region. Non-limiting examples of primers and probes for detecting Dengue are provided below and in Example 17 and FIG. 33.

Dengue Primers and Probe

| Oligo ID | Sequence (5′-3′) | 3′ |
|---|---|---|
| DENV 4 FP | CACAAAAACAGCATATTGACGCTGGGAAAG | |
| DENV 1-3 FP | AACAGCATATTGACGCTGGGAGAGACCAGAGATC | |
| DenMPExoP9FAM | CCATTTTCTGGCGTTCTGTGCCTGGAATGATG[T(FAM)]TG | C3-spacer |
| | [dSpacer][T(BHQ1)]GAGACAGCAGGAT | |
| DEV 1-3 RP | CACAGAACGCCAGAAAATGGAATGGTGCTGTTGAAT | |
| DEV 4 RP | TCAATCCAGGCACAGAGCGCCGCAAGATG | |

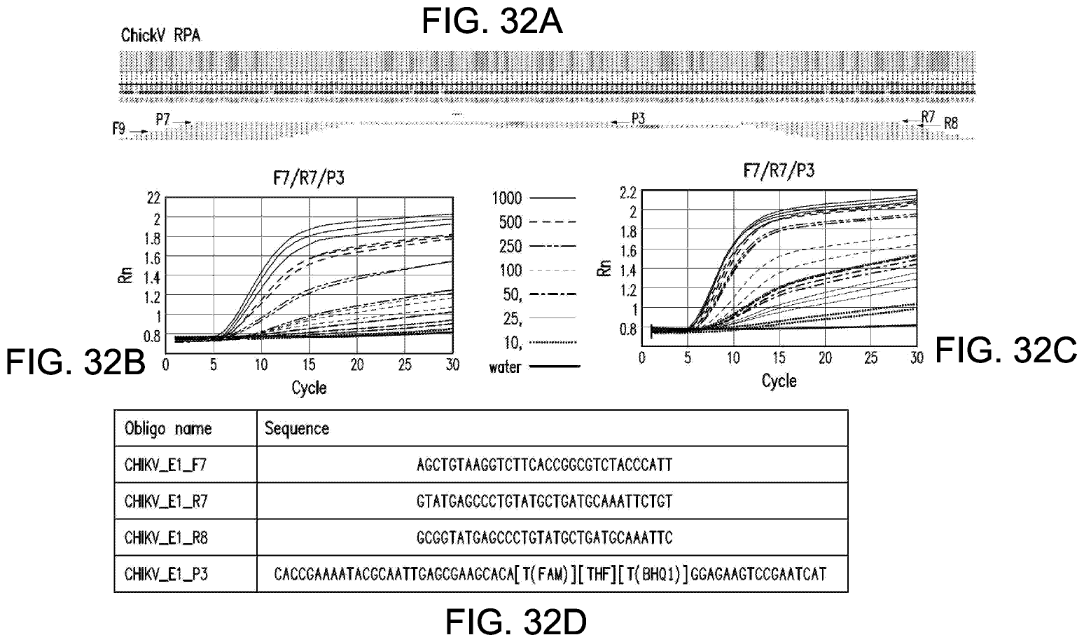

Additionally, or alternatively, the methods and system components for nucleic acid detection described herein are sufficiently sensitive to achieve a limit of detection of 1-100 copies/mL for the detection of Chikungunya. In certain embodiments, the methods and system components are sufficiently sensitive to achieve a limit of detection of 1-50 copies/mL for the detection of Chikungunya. In certain embodiments, the methods and system components are sufficiently sensitive to achieve a limit of detection of 1-25 copies/mL for the detection of Chikungunya. In certain embodiments, the methods and system components are sufficiently sensitive to achieve a limit of detection of 10 copies/mL for the detection of Chikungunya. In certain embodiments, the amplification/detection target is within the E1 gene. Non-limiting examples of primers and probes for detecting Chikungunya are provided below and in Example 16 and FIG. 32.

Chikungunya Primers and Probe

| Oligo ID | Sequence (5′-3′) |
|---|---|
| CHIKV_E1_F7 | AGCTGTAAGGTCTTCACCGGCGTCTACCCATT |
| CHIKV_E1_R7 | GTATGAGCCCTGTATGCTGATGCAAATTCTGT |
| CHIKV_E1_R8 | GCGGTATGAGCCCTGTATGCTGATGCAAATTC |
| CHIKV_E1_P3 | CACCGAAAATACGCAATTGAGCGAAGCACA[T(FAM)][THF][T(BHQ1)]GGAGAAG |
| | TCCGAATCAT |

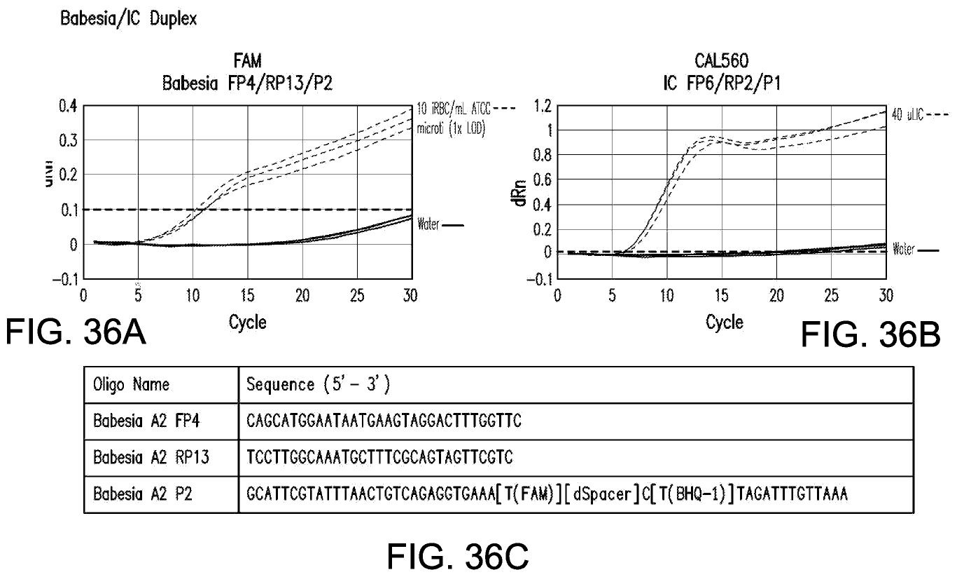

Additionally, or alternatively, the methods and system components for nucleic acid detection described herein are sufficiently sensitive to achieve a limit of detection of 1-50 copies/mL for the detection of Babesia. In certain embodiments, the methods and system components are sufficiently sensitive to achieve a limit of detection of 1-25 copies/mL for the detection of Babesia. In certain embodiments, the methods and system components are sufficiently sensitive to achieve a limit of detection of 1-15 copies/mL for the detection of Babesia. In certain embodiments, the methods and system components are sufficiently sensitive to achieve a limit of detection of 10 copies/mL for the detection of Babesia. In certain embodiments, the amplification/detection target is within the 18s rRNA gene. Non-limiting examples of primers and probes for detecting Babesia are provided below and in FIG. 36, Table 13, Example 9 and Example 20.

Babesia Primers and Probe

| Oligo | | |
|---|---|---|
| Name | Sequence (5′-3′) | 3′ |
| Babesia | CAGCATGGAATAATGAAGTAGGACTTTGGTTC | |
| A2 FP4 | | |
| Babesia | TCCTTGGCAAATGCTTTCGCAGTAGTTCGTC | |
| A2 RP13 | | |
| Babesia | GCATTCGTATTTAACTGTCAGAGGTGAAA[T(FAM)][dSpacer]C[T(BHQ- | C3-spacer |
| A2 P2 | 1)]TAGATTTGTTAAA | |

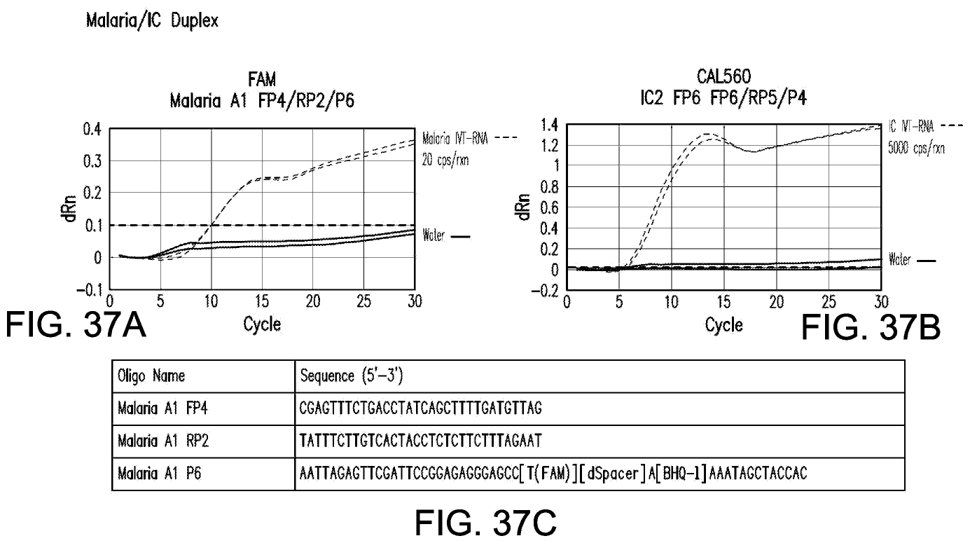

Additionally, or alternatively, the methods and system components for nucleic acid detection described herein are sufficiently sensitive to achieve a limit of detection of 1-100 copies/mL for the detection of Malaria. In certain embodiments, the methods and system components are sufficiently sensitive to achieve a limit of detection of 1-50 copies/mL for the detection of Malaria. In certain embodiments, the methods and system components are sufficiently sensitive to achieve a limit of detection of 1-25 copies/mL for the detection of Malaria. In certain embodiments, the methods and system components are sufficiently sensitive to achieve a limit of detection of 10 copies/mL for the detection of Malaria. In certain embodiments, the amplification/detection target is within the 18s rRNA gene. Non-limiting examples of primers and probes for detecting Malaria are provided below and in Example 21 and FIG. 37.

Malaria Primers and Probe

| Oligo Name | Sequence (5′-3′) |
|---|---|
| Malaria A1 | CGAGTTTCTGACCTATCAGCTTTTGATGTTAG |
| FP4 | |
| Malaria A1 | TATTTCTTGTCACTACCTCTCTTCTTTAGAAT |
| RP2 | |
| Malaria A1 | AATTAGAGTTCGATTCCGGAGAGGGAGCC[T(FAM)][dSpacer]A[BHQ- |
| P6 | 1]AAATAGCTACCAC |

Additionally, or alternatively, the methods and system components for nucleic acid detection described herein are sufficiently sensitive to achieve a limit of detection of 1-40 IU/mL for the detection of HAV. In certain embodiments, the methods and system components are sufficiently sensitive to achieve a limit of detection of 1-20 IU/mL for the detection of HAV. In certain embodiments, the methods and system components are sufficiently sensitive to achieve a limit of detection of 1-10 IU/mL for the detection of HAV. In certain embodiments, the methods and system components are sufficiently sensitive to achieve a limit of detection of 5 IU/mL for the detection of HAV. In certain embodiments, the amplification/detection target is within the 5′ UTR region. Non-limiting examples of primers and probes for detecting HAV are provided below and in FIG. 30, Table 12, Example 8 and Example 23.

HAV Target (5′ UTR):
Oligo ID

HAV-P1
HAV-FP3
HAV-RP2

FPs for HAV 5′UTR:
Oligo ID

HAV-FP5
HAV-FP6

RPs for HAV 5′UTR:
Oligo ID

HAV-RP1
HAV-RP4
HAV-RP5

Probes for HAV 5'UTR:
Oligo ID
HAV-P2

Additionally, or alternatively, the methods and system components for nucleic acid detection described herein are sufficiently sensitive to achieve a limit of detection of 1-40 IU/mL for the detection of HEV. In certain embodiments, the methods and system components are sufficiently sensitive to achieve a limit of detection of 1-20 IU/mL for the detection of HEV. In certain embodiments, the methods and system components are sufficiently sensitive to achieve a limit of detection of 1-10 IU/mL for the detection of HEV. In certain embodiments, the methods and system components are sufficiently sensitive to achieve a limit of detection of 5 IU/mL for the detection of HEV. In certain embodiments, the amplification/detection target is within the 5′ UTR of ORF1 and/or ORF2/3. Non-limiting examples of primers and probes for detecting HEV are provided below.

HEV Forward Primers

| Oligo Name | Sequence (5′-3′) |
|---|---|
| HEV A1 FP1 | CCATCGCCTATGCCTTATGTCCCTTACCC |
| HEV A1 FP2 | ATGCCTTATGTCCCTTACCCTCGTTCAAC |
| HEV A1 FP3 | TATGTCCCTTACCCTCGTTCAACCGAGGT |
| HEV A2 FP1 | CGTCTGGCCGCCGTCGTGGGCGGCGCAGCGG |
| HEV A2 FP2 | TGGCCGCCGTCGTGGGCGGCGCAGCGGCGG |
| HEV A2 FP3 | CGCCGTCGTGGGCGGCGCAGCGGCGGTGCC |
| HEV A2 FP4 | CGTCGTGGGCGGCGCAGCGGCGGTGCCGGC |
| HEV A3 FP1 | CCGACAGAATTGATTTCGTCGGCTGG |
| HEV A3 FP2 | CAGAATTGATTTCGTCGGCTGGTGGTCA |
| HEV A3 FP3 | ATTGATTTCGTCGGCTGGTGGTCAGCTGTT |
| HEV A3 FP4 | TTTCGTCGGCTGGTGGTCAGCTGTTTTACTC |

HEV Reverse Primers

| Oligo Name | Sequence (5′-3′) |
|---|---|
| HEV A1 RP1 | AATGTAGACTTAGTAGAGCAGGCTGATGG |
| HEV A1 RP2 | ACAGCATGAAATGTAGACTTAGTAGAGCA |
| HEV A1 RP3 | TCCCAGATATGCACAGGGACAGCATG |
| HEV A2 RP1 | AAGGGGTTGGTTGGATGAATATAGGGGA |
| HEV A2 RP2 | GCGAAGGGGTTGGTTGGATGAATATAGG |
| HEV A2 RP3 | TCGGAGGCGAAGGGGTTGGTTGGATGAATA |
| HEV A3 RP1 | ATACCCTTATCCTGCTGAGCATTCTCGACA |
| HEV A3 RP2 | ATAGCAATACCCTTATCCTGCTGAGCATTCTC |
| HEV A3 RP3 | GACTCACCAAGGTCTATATCATGTGGGAT |
| HEV A3 RP4 | ACACGGGACTCACCAAGGTCTATATCATGTGG |

HEV Probes

| Oligo Name | Sequence (5′-3′) |
|---|---|
| HEV A1 P1 | TGTCCCTTACCCTCGTTCAACCGAGGTGTATGTTCGGTCCATATT |
| HEV A1 P2 | CCTCGTTCAACCGAGGTGTATGTTCGGTCCATATTTGGCCCTGGTGG |
| HEV A1 P3 | TCCATATTTGGCCCTGGTGGTTCCCCATCCCTGTTTCCATCAGCCTG |
| HEV A2 P1 | CCGGCGGTGGTTTCTGGGGTGACCGGGTTGATTCTCAGCCCTTCGC |
| HEV A2 P2 | GTGGTTTCTGGGGTGACCGGGTTGATTCTCAGCCCTTCGCCCTCCCCTA |
| HEV A3 P1 | TACTCCCGCCCCGTCGTCTCAGCCAATGGCGAGCCGACTGTGAA |
| HEV A3 P2 | GTCGTCTCAGCCAATGGCGAGCCGACTGTGAAGCTATACACATC |

Additionally, or alternatively, the methods and system components described herein include an internal control, e.g., an internal control nucleic acid as described herein. In certain embodiments, a 351-base sequence of the hydroxypyruvate reductase gene (HPR) from the pumpkin plant that is unrelated to the HIV-1, HIV-2, HBV, HCV, Babesia, Malaria, WNV, Zika, Chikungunya, Dengue, HAV, Parvo B19 and HEV sequence and is encapsulated within a bacteriophage protein along with coliphage MS2 packaging sequences (armored RNA) to serve as the target for an internal control. Non-limiting examples of primers and probes for detecting an internal control are provided below.

Internal Control

| Oligo | | |
|---|---|---|
| Name | Sequence | 3′ |
| IC FP1 | 5′-CAGAAACTACAGCAGAGTTGGCAGCTTCAC-3′ | |
| IC RP2 | 5′-GTCTGGCCTTTCAGCAAGTTTCCAACAAACA-3′ | |
| IC P1 | 5′-AGCTGACGAGTTCATGAGGGCAGGCCGC-[T(CAL | C3-spacer |
| | Fluor Orange 560)]-A-[dSpacer]-GA-[T(BHQ- | |
| | 1)]-GGATGGCTTCCAA-3′ | |

Additionally, or alternatively, the methods and system components for nucleic acid detection described herein are sufficiently sensitive to achieve a limit of detection of 1-50 copies/mL of SARS-CoV-2 (COVID-19). In certain embodiments, the methods and system components achieve a limit of detection of 1-25 copies/mL for the detection of SARS-CoV-2 (COVID-19). In certain embodiments, the methods and system components achieve a limit of detection of 20 copies/mL for the detection of SARS-CoV-2 (COVID-19). In certain embodiments, the biological sample used for detection of SARS-CoV-2 (COVID-19) will be saliva, sweat, tears, mucus, urine or any other sample suitable for analysis using the methods and techniques described herein. In certain embodiments, the sample will be obtained using a swab, e.g., a nasopharyngeal swab, and the swab will be contacted with sample transfer buffer to obtain a sample that can be aspirated into the systems of the present disclosure. In certain embodiments, the amplification/detection target is within the RdRp and/or N genes. Non-limiting examples of primers and probes for detecting SARS-CoV-2 (COVID-19) are provided below and in Example 22.

| Oligo Name | Sequence | 3′ Mod |
|---|---|---|
| COVID-19 FP1 | TAACATGCTTAGAATTATGGCCTCACTTGTTC | |
| COVID-19 FP2 | AAAGAAGAAGGCTGATGAAACTCAAGCCTTAC | — |
| COVID-19 FP3 | GCTGATGAAACTCAAGCCTTACCGCAGAGACA | — |
| COVID-19 RP1 | TGACCATTTCACTCAATACTTGAGCACACTC | — |
| COVID-19 RP2 | ACTGCTCATGGATTGTTGCAATTGTTTGGAGA | — |
| COVID-19 RP3 | TTGAGTCAGCACTGCTCATGGATTGTTGCAAT | — |
| COVID-19 P1 | CTTGCTCGCAAACATACAACGTGTTGTAGC[T(FAM)][dSpacer] | Spacer-C3 |
| | G[T(BHQ-1)]CACACCGTTTCTAT | |
| COVID-19 P2 | AGCCTTACCGCAGAGACAGAAGAAACAGCAAAC[T(FAM)] | Spacer-C3 |
| | [dspacer][T(BHQ-1)]GACTCTTCTTCCTG | |
| COVID-19 P3 | CGCAGAGACAGAAGAAACAGCAAACTGTGAC[T(FAM)]C | Spacer-C3 |
| | [dspacer][T(BHQ-1)]CTTCCTGCTGCAGA | |
| COVID-19 P4 | AGACAGAAGAAACAGCAAACTGTGACTCTTCT[T(FAM)]C | Spacer-C3 |
| | [dspacer][T(BHQ-1)]GCTGCAGATTTGGA | |

Additionally, or alternatively, the methods and system components for nucleic acid detection described herein are sufficiently sensitive to achieve a limit of detection of 10-50 IU/mL for the detection of CMV. In certain embodiments, the methods and system components are sufficiently sensitive to achieve a limit of detection of 10-25 IU/mL for the detection of CMV. In certain embodiments, the methods and system components are sufficiently sensitive to achieve a limit of detection of 10 IU/mL for the detection of CMV. In certain embodiments, the biological sample used for detection of CMV will be saliva, sweat, tears, mucus, urine or any other sample suitable for analysis using the methods and techniques described herein. In certain embodiments, the sample will be obtained using a swab, e.g., a nasopharyngeal swab, and the swab will be contacted with sample transfer buffer to obtain a sample that can be aspirated into the systems of the present disclosure.

Additionally, or alternatively, the methods and system components for nucleic acid detection described herein are sufficiently sensitive to achieve a limit of detection of 10-1000 copies/mL for the detection of Influenza. In certain embodiments, the methods and system components are sufficiently sensitive to achieve a limit of detection of 10-500 copies/mL for the detection of Influenza. In certain embodiments, the methods and system components are sufficiently sensitive to achieve a limit of detection of 10-250 copies/mL for the detection of Influenza. In certain embodiments, the methods and system components are sufficiently sensitive to achieve a limit of detection of 100 copies/mL for the detection of Influenza. In certain embodiments, the biological sample used for detection of Influenza will be saliva, sweat, tears, mucus, urine or any other sample suitable for analysis using the methods and techniques described herein. In certain embodiments, the sample will be obtained using a swab, e.g., a nasopharyngeal swab, and the swab will be contacted with sample transfer buffer to obtain a sample that can be aspirated into the systems of the present disclosure.

Additionally, or alternatively, the methods and system components for nucleic acid detection described herein are sufficiently sensitive to achieve a limit of detection of 100-1000 copies/mL for the detection of Chlamydia. In certain embodiments, the methods and system components are sufficiently sensitive to achieve a limit of detection of 100-500 copies/mL for the detection of Chlamydia. In certain embodiments, the methods and system components are sufficiently sensitive to achieve a limit of detection of 100-250 copies/mL for the detection of Chlamydia. In certain embodiments, the methods and system components are sufficiently sensitive to achieve a limit of detection of 200 copies/mL for the detection of Chlamydia. In certain embodiments, the biological sample used for detection of Chlamydia will be saliva, sweat, tears, mucus, urine or any other sample suitable for analysis using the methods and techniques described herein. In certain embodiments, the sample will be obtained using a swab, e.g., a nasopharyngeal swab, and the swab will be contacted with sample transfer buffer to obtain a sample that can be aspirated into the systems of the present disclosure.

Additionally, or alternatively, the methods and system components for nucleic acid detection described herein are sufficiently sensitive to achieve a limit of detection of 100-500 copies/mL for the detection of Gonorrhea. In certain embodiments, the methods and system components are sufficiently sensitive to achieve a limit of detection of 100-250 copies/mL for the detection of Gonorrhea. In certain embodiments, the methods and system components are sufficiently sensitive to achieve a limit of detection of 200 copies/mL for the detection of Gonorrhea. In certain embodiments, the biological sample used for detection of Gonorrhea will be saliva, sweat, tears, mucus, urine or any other sample suitable for analysis using the methods and techniques described herein. In certain embodiments, the sample will be obtained using a swab, e.g., a nasopharyngeal swab, and the swab will be contacted with sample transfer buffer to obtain a sample that can be aspirated into the systems of the present disclosure.

### 5. Methods of Use

For purpose of understanding, and not limitation, reference will now be made in detail to various exemplary methods and aspects thereof of the disclosed subject matter, which are illustrated in the accompanying drawings and described in greater detail below. Each of the aspects as described above, e.g., sample preparation aspects, amplification aspects, and detection aspects, of the disclosed subject matter provide various benefits and it is recognized the various aspects and corresponding system components can be selectively combined to achieve the desired benefits of the methods and systems disclosed herein.

Increasing throughput and speed often sacrifices other essential or highly desired features of sample analysis, particularly where complicated samples such as blood or tissue are used, where molecular assays are employed, where the analytes to be detected are a minor component of the complicated sample, and where multi-component instrumentation is used. Increases in throughput and speed can lead to losses in sensitivity, specificity, and limit of detection. They can also lead to increases in errors and mistakes. In the field of donor blood testing, errors and mistakes and reductions in sensitivity, specificity, and limit of detection can have significant consequences, increasing the chances that a subject is infected by a clinical product or process, causing undo loss of important clinical materials, and/or causing loss of confidence in a critical health care system. Existing systems, which are designed to be as fast and efficient as possible, while providing the sensitivity, specificity, limit of detection, and reliability required in this field lack the throughput, speed, size, and cost profile necessary to allow such testing in diverse locations, such as point-of-collection of donor samples. Rather, the majority of such testing occurs at dedicated screening centers that are often remote from donor collection locations. Preparation for transport and transport of samples further increases the cost and time to result. To date, there are no available high throughput, fast, and cost-effective systems that have sufficient accuracy and reliability, for detection of complex analytes in complex test samples, that have facilitated adaptation of routine testing of donor blood samples outside of specialized screening facilities. The systems and methods described herein have overcome the competing, and historically unsolved challenges of speed/throughput versus accuracy/reliability and provide fast, high-throughput, sensitive, specific, cost-effective solutions that permit detection of multiple complex analytes, at low limits of detection, in complex sample types. This solution makes it feasible to conduct donor blood sample screening at a wide range of locations, including at point-of-collection locations, as well as local and/or regional locations where donor blood is held prior to release to blood banks.

The high-throughput NAT-based screening methods and systems described herein find use in a wide variety of indications and operational arrangements. For example, but not by way of limitation, the methods and systems described herein find use in the screening of donor blood for release of the donor blood or release of a donor material for clinical use. Currently, donor blood is held, often for extended periods of time, while samples related to the donor blood are screened for the presence of one or more pathogens or infectious agents. Generally, such donor blood is released for clinical use only upon the completion of such screening, which can also include additional screening aspects, e.g. HLA typing and blood group identification, among other potential screening steps. Thus, the high-throughput methods and systems of the present disclosure are particularly adapted, via specific performance attributes, e.g., time to result and throughput per unit size of the systems, to accelerate the release of such samples. In addition, the methods and systems of the present disclosure also provide process options for screening of the samples, e.g., prioritizing specific samples and/or the screening of specific pathogens or infectious agents, that can operationally enhance donor and patient safety and access.

Additionally, or alternatively, the systems described herein can provide particular advantages based on their deployment relative to existing blood collection, storage, and use paradigms. As noted herein, donor blood is typically collected as whole blood or, using apheresis, as plasma. Collection of both whole blood and plasma are typically collected at blood and plasma collection locations local to the donors. Whole blood donations are sent to local blood centers, or held at the local blood centers if the blood center was the collection location, for further processing such as centrifugation to produce RBCs, platelets, and/or plasma. The contemporaneously collected samples, however, are transported to a regional, or centralized, screening facility that can be multiple hours away from the blood center. The processed blood products are held at the blood center until the contemporaneously collected samples are screened. The logistics of transporting the sample to the centralized screening center, however, cause delay in the ability of the donated blood to be used. Pick-up times for the samples from blood centers may be once per day and may be in the evening. Thus, where blood was collected early in the day, hours may elapse before a sample is even picked up for screening. As mentioned above, after pickup, the testing center may be hours away from the blood center, causing further delay causing further delay in the testing and release of blood. Further, since pick-up times are often in the evening, the samples will not arrive at the testing center until late in the date, after the testing center has closed. Thus, testing does not happen typically occur until the day after the donation. Because testing can currently take a day to complete, test results may not be available until late the next day. As a further complication, hospitals place orders for blood products earlier in a day and before sample testing may be complete. As a result, samples that have not been tested completely are not available for order and the associated donor blood will have to wait another day to be available for ordering. Given the extremely short shelf life of certain blood products, e.g., platelets have a 5-day shelf life, the transport time and other logistical issues associated with centralization of NAT-based screening of the contemporaneously collected samples can significantly impact the efficiency of the system, with a material amount of platelets unused by their expiration date. Accordingly, deployment of the systems described herein at the local blood centers, can eliminate the delays in release associated with transport time, as well as conventional NAT-based screening time, to significantly reduce the window of time between collection of donor blood and its availability for use.

With this general understanding, for example, and not limitation, in accordance with one aspect of the disclosed subject matter, the methods and systems of screening donor blood samples can occur at a blood center where the blood is processed to produce blood components. Donor blood can be released from the blood center without transportation of the samples associated with the donor blood to a distant testing center. In some embodiments, donor blood may be subjected to NAT screening at the blood center on the same day as the donor blood being received by the blood center. In some embodiments, donor blood can undergo NAT screening to obtain a NAT result related to the release of donor blood within eight hours of the donor blood being received at the blood center. In some embodiments, hospitals can order blood received at the blood center on the same day.

In contrast to the current decentralized storage of whole blood donations, plasma donations and the samples collected contemporaneously with such plasma donation are transported together to centralized plasma centers. Thus, deployment of the systems of the present disclosure at the plasma collection center can provide for improved efficiency of the overall paradigm, e.g., by screening donors during the plasma apheresis process, thereby avoiding the pooling and potential pool deconstruction currently faced by plasma screening, or by providing a result such that once the plasma reaches the centralized facility, no further NAT-based screening delay will impede its release for pooling and fractionation into plasma-derived products.

### 5.1 Release of Donor Blood or Donor Material

In certain embodiments, the methods and systems described herein can be used for the screening of a sample of donor blood for release of the donor blood or a donor material for clinical use. Screening of a sample of donor blood for release of the donor blood or the donor material for clinical use as used herein refers to the performance of a nucleic acid analysis on a sample of donor blood to detect one or more pathogens or infectious agents. In certain embodiments, the determination of the presence or absence of nucleic acids derived from the pathogens or infectious agents is indicative of whether the donor blood or donor material can be released. In certain embodiments, a determination of the presence or absence of predetermined level of nucleic acids derived from the pathogens or infectious agents is indicative of whether the donor blood or donor material can be released.

In general, release of the donor blood or donor material is predicated on a negative result with respect to a set of one or more pathogens or infectious agents as well as the collection of additional information concerning the donor blood or donor material, e.g., HLA type and blood group testing. Accordingly, the methods of the present disclosure for donor blood release or donor material release can comprise additional screening steps beyond the HTNAT screening described herein. For example, but not by way of limitation, such additional screening can include blood group testing and/or HLA tissue typing, among other release tests.

Additionally, or alternatively, the methods and systems of the present disclosure for the screening of a sample of donor blood for release of that donor blood or a donor material for clinical use comprise screening of a sample of donor blood where the sample is of human donor blood. For example, in certain embodiments, the sample of donor blood is whole blood. In certain embodiments, the sample of donor blood is lysed whole blood. In certain embodiments, the sample of donor blood is serum. In certain embodiments, the sample of donor blood is plasma.

Additionally, or alternatively, the release of donor blood for clinical use is a release for use in connection with a transfusion. In certain embodiments, the release of donor blood for clinical use is a release for use in a pharmaceutical. In certain embodiments, the release of donor blood for clinical use is a release for use in a therapeutic treatment. In certain embodiments, the release of donor blood for clinical use is a release for use as a blood donation.

In certain embodiments, the release of donor material for clinical use is a release for use in connection with a transfusion. In certain embodiments, the release of donor material for clinical use is a release for use in a pharmaceutical. In certain embodiments, the release of donor material for clinical use is a release for use in a therapeutic treatment. In certain embodiments, the release of donor material for clinical use is a release for use as a blood donation.

In accordance with the disclosed subject matter, the methods and systems of the present disclosure for the screening of a sample of donor blood for release of that donor blood or a material from that donor for clinical use comprise: performing a nucleic acid analysis on the sample of donor blood to detect a plurality of pathogens or infectious agents. In certain embodiments, the determination of the presence or absence of nucleic acids derived from the pathogens or infectious agents is indicative of whether the donor blood or donor material can be released. In certain embodiments, a determination of a predetermined level of nucleic acids derived from each of the plurality of pathogens or infectious agents based on the nucleic acid analysis, is indicative of release of the donor blood or donor material for clinical use. For example, but not by way of limitation, a nucleic acid analysis determination that at least the predetermined level of nucleic acids derived from one or more of the plurality of pathogens or infectious agents is present in the donor blood sample, indicates that the donor blood or donor material cannot be released for clinical use (e.g., it can be quarantined for confirmatory testing, destroyed, or released for non-clinical use). In certain embodiments, a nucleic acid analysis determination that nucleic acids derived from one or more of the plurality of pathogens or infectious agents is present in the donor blood sample indicates that the donor blood or donor material cannot be released for clinical use (e.g., it can be quarantined for confirmatory testing, destroyed, or released for non-clinical use). Conversely, a nucleic acid analysis determination that the predetermined level of nucleic acids derived from one or more of the plurality of pathogens or infectious agents is not present in the donor blood sample, indicates that the donor blood or donor material can be further considered for release for clinical use. As noted above, such further consideration can include blood group testing and/or HLA tissue typing, among other release tests.

In certain embodiments, the methods and systems of the present disclosure for the screening of a sample of donor blood for release of that donor blood or a material from that donor for clinical use comprise: performing only a nucleic acid analysis on the sample of donor blood to detect a plurality of pathogens or infectious agents. In certain embodiments, a determination of a predetermined level of nucleic acids derived from each of the plurality of pathogens or infectious agents based on the nucleic acid analysis, is indicative of release of the donor blood or donor material for clinical use. In certain embodiments, the determination of the presence or absence of nucleic acids derived from the pathogens or infectious agents is indicative of whether the donor blood or donor material can be released for clinical use.

In certain embodiments, determining a level of the pathogen or infectious agent at or above a predetermined level is equivalent to detecting the presence of the pathogen or infectious agent. In certain embodiments, determining a level of the pathogen or infectious agent below a predetermined level is equivalent to determining the absence of the pathogen or infectious agent.

In certain embodiments, the methods and systems of the present disclosure for the screening of a sample of donor blood for release of that donor blood for clinical use comprise: performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis, is indicative of release of the donor blood for clinical use in the absence of immunoassay analysis of the donor blood.

In certain embodiments, the methods and systems of the present disclosure for the screening of a sample of donor blood, e.g., for release of that donor blood or a donor material for clinical use, can further comprise additional screening of the sample using, for example, serologic testing. Additionally or alternatively, the methods and systems of the present disclosure for the screening of a sample of donor blood, e.g., for release of that donor blood or a material from that donor for clinical use, can further comprise treating the donor blood or donor material using pathogen reduction technologies.

### 5.2 Release Methods Capable of Certain Performance Criteria

In accordance with another aspect of the disclosed subject matter, the methods and systems of the present disclosure for donor blood release or donor material release comprise screening of a sample of donor blood where the assay is capable of achieving certain performance criteria. Performance criteria as used herein, refers to assay parameters associated with the duration of one or more step of the assay. For example, but not by way of limitation, performance criteria can include the duration of an amplification reaction, the time to result for a single assay or for a plurality of assays, as well as the throughput of one or more assays as a function of the size of the system (e.g., samples analyzed per hour per m² of a footprint of the automated system).

For example, and not limitation, in accordance with one aspect of the disclosed subject matter, the methods and systems of screening donor blood samples can perform a nucleic acid analysis on a sample to detect one or a plurality of pathogens or infectious agents, where a determination of a predetermined level of nucleic acids derived from each of the pathogens or infectious agents can be based on the nucleic acid analysis performed, the determination can be indicative of whether the donor blood or a donor material can be released for clinical use, and the release of the donor blood or the donor material for clinical use can occur in about 15 to about 60 minutes, e.g., about 20 to about 60 minutes, about 20 minutes to about 45 minutes, about 34 minutes or about 39 minutes, from initial aspiration of the sample for performance of the nucleic acid analysis in accordance with the disclosed subject matter. Additionally, the methods and systems can determine whether the donor blood is acceptable for transfusion based in part on the nucleic acid analysis result.

Additionally, or alternatively, and in accordance with another aspect of the disclosed subject matter, the methods and systems of screening donor blood samples can perform a nucleic acid analysis on a sample to detect one or a plurality of pathogens or infectious agents, where the determination of a predetermined level and/or the determination of the absence or presence of nucleic acids derived from each of the pathogens or infectious agents can be based on the nucleic acid analysis performed, and where the nucleic acid analysis includes a nucleic acid amplification reaction (e.g., an amplification and detection process) in accordance with the disclosed subject matter of about 1 minute to about 20 minutes, e.g., about 8 minutes to about 20 minutes in duration. The determination can be indicative of whether the donor blood or donor material can be released for clinical use. Additionally, the methods and systems can determine whether the donor blood is acceptable for transfusion based in part on the nucleic acid analysis result. In certain embodiments, the methods and systems can determine whether a material derived from the donor is acceptable for transplantation or for production of a therapeutic based in part on the nucleic acid analysis result.

Additionally, or alternatively, and in accordance with another aspect of the disclosed subject matter, the methods and systems of screening donor blood samples can perform a nucleic acid analysis on a sample to detect one or a plurality of pathogens or infectious agents, where the determination of a predetermined level and/or the determination of the absence or presence of nucleic acids derived from each of the pathogens or infectious agents can be based on the nucleic acid analysis performed. In certain embodiments, each determination of the predetermined level of nucleic acid from one of the pathogens or infectious agents is completed in about 20 to about 45 minutes from initial aspiration of the sample for performance of the nucleic acid analysis in accordance with the disclosed subject matter. In certain embodiments, each determination of absence or presence of nucleic acid from one of the pathogens or infectious agents is completed in about 15 to about 45 minutes, e.g., about 20 to about 45 minutes, from initial aspiration of the sample for performance of the nucleic acid analysis in accordance with the disclosed subject matter. The determination can be indicative of whether the donor blood or a donor material can be released for clinical use. Additionally, the methods and systems can determine whether donor blood is acceptable for transfusion based in part on the nucleic acid analysis result. In certain embodiments, the methods and systems can determine whether a material derived from the donor is acceptable for transplantation or for production of a therapeutic based in part on the nucleic acid analysis result.

Additionally, or alternatively, and in accordance with another aspect of the disclosed subject matter, the methods and systems of screening donor blood samples can perform a nucleic acid analysis on a sample to detect one or a plurality of pathogens or infectious agents, where the determination of a predetermined level of nucleic acids derived from each of the pathogens or infectious agents can be based on the nucleic acid analysis performed, and where each determination of the predetermined level of nucleic acid from one of the pathogens or infectious agents has a time to result of about 20 to about 45 minutes. The determination can be indicative of whether the donor blood can be released for clinical use. Additionally, the methods and systems can determine whether donor blood is acceptable for transfusion based in part on the nucleic acid analysis result. In certain embodiments, the methods and systems can determine whether a material derived from the donor can be released for clinical use based in part on the nucleic acid analysis result.

Additionally, or alternatively, and in accordance with another aspect of the disclosed subject matter, the methods and systems of screening donor blood samples can perform a nucleic acid analysis on a plurality of samples to detect one or a plurality of pathogens or infectious agents. A determination of a predetermined level of nucleic acids derived from each of the pathogens or infectious agents in each of the plurality of samples can be based on the nucleic acid analyses performed. The determinations based on the nucleic acid analyses are performed within about 20 minutes to about 3.5 hours from initial aspiration of the first sample for performance of the nucleic acid analysis in accordance with the disclosed subject matter. The determination can be indicative of whether the donor blood can be released for clinical use. Additionally, the methods and systems can determine whether the donor blood is acceptable for transfusion based in part on the nucleic acid analysis result. In certain embodiments, the methods and systems can determine whether a material derived from the donor can be released for clinical use based in part on the nucleic acid analysis result.

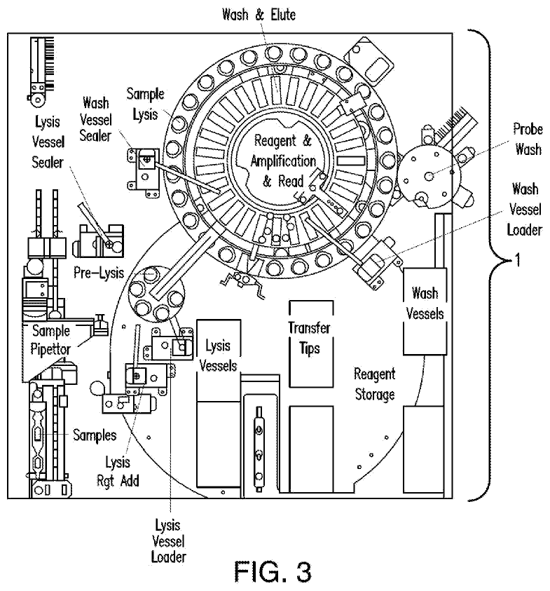

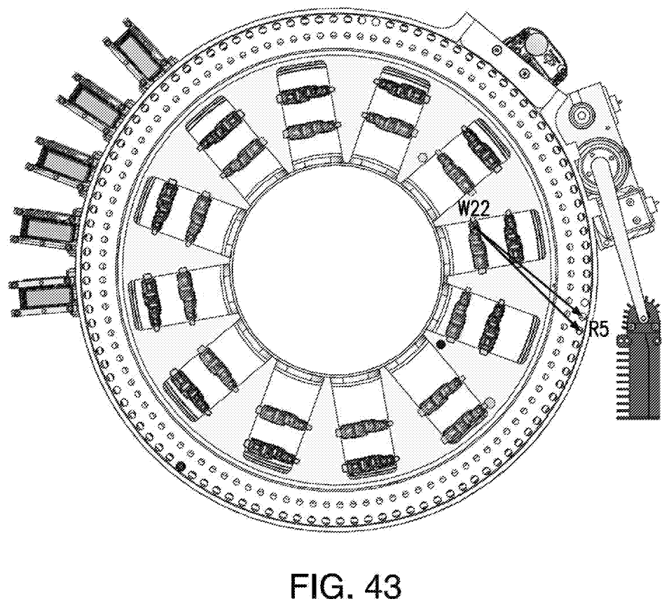

In accordance with another aspect of the disclosed subject matter, the release of donor blood or donor material for clinical use based on the methods and systems of the present disclosure can achieve certain performance criteria relating to sample throughput (as defined as the number of nucleic acid analysis results per hour) per unit of area or volume occupied by the system, e.g., the “efficiency” of the system. For example, but not limitation, the systems embodied herein, e.g., in FIGS. 3, 5-8, 15-16, 43, and 45-66, can have a footprint of about 1 m² to about 3 m², or about 1 m² to about 2.5 m², or about 1 m² to about 2 m², 1 m² to about 1.5 m² or even about 1 m² to about 1.2 m². As embodied herein, such systems will generally have a height of less than 6 feet (about 2 m), and, in certain embodiments, will be configured to have a height not more than that associated with 90% of the population intended to operate the system. In certain embodiments, the systems of the present disclosure can be configured to have a height not more than that associated with 90% of the female population intended to operate the system. Thus, in certain embodiments, the systems embodied herein, e.g., in FIGS. 3, 5-8, 15-16, 43, and 45-66, can have a volume of about 1 m³ to about 3 m³, or about 1 m³ to about 3.5 m³, or about 1 m³ to about 3 m³, or about 1 m³ to about 2.5 m³, or even about 1 m³ to about 2 m³ based on a height of about 1 m to about 2 m.

In accordance with another aspect of the disclosed subject matter, the methods of the present disclosure can be employed in the context of a system configured for multi-sample analysis and thus the ability of the methods and systems of the present disclosure to perform high efficiency multi-sample analysis within a limited footprint will provide significant benefits over existing blood screening systems. Thus, high efficiency systems, e.g., systems that achieve at least 30 nucleic acid analysis results per hour per m² or at least 17 nucleic acid analysis results per hour per m³ (where the system has a footprint of about 1 m² to about 2 m² (and/or a volume of about 1 m³ to about 4 m³ based on a height of about 1 m to about 2 m), provide material benefits over existing systems with respect to ease of installation, ease of operator use, and overall resource requirements (e.g., costs associated with dedicated laboratory space).

The following table, Table 1, outlines efficiencies associated with various embodiments of the methods of the systems having distinct area, volume, and throughput as measured in results per hour. As discussed herein term “results” refers to the number determinations of the presence or absence of a target nucleic acid derived from a pathogen or infectious agent, or a predetermined amount thereof, made by a nucleic acid analysis. For example, but not limitation, the systems described herein are capable of aspirating a sample into sample preparation subsystem every 24 seconds, leading to 150 sample aspirations per hour. In certain embodiments, each sample aspirated will be prepared using the sample preparation subsystem and a single target nucleic acid will be amplified and detected, leading to 150 individual results per hour, after the initial start-up period during with the initial sample aspirated is prepared, amplified and detected. In certain embodiments, each aspirated sample will be prepared and split into two amplifications, where each amplification reaction amplifies a single target nucleic acid for detection, leading to 300 individual results per hour, after the initial start-up period during with the initial sample aspirated is prepared, amplified and detected. In certain embodiments, each aspirated sample will be prepared and split into two amplifications, where each amplification reaction amplifies two or more target nucleic acids for detection (e.g., duplex, triplex, or other higher order multiplex amplifications and detections), leading to 600 or more individual results per hour, after the initial start-up period during with the initial sample aspirated is prepared, amplified and detected. In certain embodiments, that initial start-up time will be 20-45 minutes, regardless of whether the elution is split into two amplifications or the amplifications are multiplexed.

TABLE 1

| Results | | | | |
|---|---|---|---|---|
| per Hour | Width | Depth | Height | Efficiency |
| Efficiency square meter (Footprint) | | | | (Results/hr/m²) |
| 150 | 1 m | 1 m | | 150 |
| 300 | 1 m | 1 m | | 300 |
| 600 | 1 m | 1 m | | 600 |
| 150 | 1 m | 1.2 m | | 125 |
| 300 | 1 m | 1.2 m | | 250 |
| 600 | 1 m | 1.2 m | | 500 |
| 150 | 1.2 m | 1.2 m | | 104 |
| 300 | 1.2 m | 1.2 m | | 208 |
| 600 | 1.2 m | 1.2 m | | 417 |
| 150 | 1.5 m | 1.5 m | | 67 |
| 300 | 1.5 m | 1.5 m | | 133 |
| 600 | 1.5 m | 1.5 m | | 267 |
| Efficiency cubic meter (Volume) | | | | (Results/hr/m³) |
| 150 | 1 m | 1 m | 1 m | 150 |
| 300 | 1 m | 1 m | 1 m | 300 |
| 600 | 1 m | 1 m | 1 m | 600 |
| 150 | 1 m | 1.2 m | 1 m | 125 |
| 300 | 1 m | 1.2 m | 1 m | 250 |
| 600 | 1 m | 1.2 m | 1 m | 500 |
| 150 | 1.2 m | 1.2 m | 1 m | 104 |
| 300 | 1.2 m | 1.2 m | 1 m | 208 |
| 600 | 1.2 m | 1.2 m | 1 m | 417 |
| 150 | 1.5 m | 1.5 m | 1 m | 67 |
| 300 | 1.5 m | 1.5 m | 1 m | 133 |
| 600 | 1.5 m | 1.5 m | 1 m | 267 |
| 150 | 1 m | 1 m | 2 m | 75 |
| 300 | 1 m | 1 m | 2 m | 150 |
| 600 | 1 m | 1 m | 2 m | 300 |
| 150 | 1 m | 1.2 m | 2 m | 63 |
| 300 | 1 m | 1.2 m | 2 m | 125 |
| 600 | 1 m | 1.2 m | 2 m | 250 |
| 150 | 1.2 m | 1.2 m | 2 m | 52 |
| 300 | 1.2 m | 1.2 m | 2 m | 104 |
| 600 | 1.2 m | 1.2 m | 2 m | 208 |
| 150 | 1.5 m | 1.5 m | 2 m | 33 |
| 300 | 1.5 m | 1.5 m | 2 m | 67 |
| 600 | 1.5 m | 1.5 m | 2 m | 133 |

The exemplary efficiencies associated with the methods and systems embodied by the present disclosure presented in the above table underscore the significant benefits associated with the methods and systems of the present disclosure when compared to the systems currently marketed. For example, but not limitation, one currently marketed NAT-based screening system has a footprint of 1.1 m² (e.g., 1.2 m×0.9 m) and an 8 hour throughput of 275 results, leading to an 8-hour efficiency (including start-up time) of 250 results per 8 hours per m². Other currently marketed NAT-based screening systems have footprints of 3.6 m² (e.g., 3 m×1.2 m) or 5.2 m² (e.g., 4.3 m×1.2 m), and 8-hour throughput of 384 results and 960 results (including start-up), respectively. Thus, these additional examples of currently marketed NAT-based screening systems have 8-hour efficiencies of 107 results per 8 hours per m² and 184 results per 8 hours per m², respectively. In contrast, the methods and systems embodied herein can achieve, 1,113 results per 8 hours (including startup), and at a footprint of 1.4 m² (e.g., 1.2 m×1.2 m), would achieve an 8-hour efficiency of 795 results per 8 hours per m². In certain embodiments, the methods and systems embodied herein can achieve, 1,113 results per 8 hours (including startup), and at a footprint of approximately 1.94 m² (e.g., approximately 1.32 m×1.47 m), would achieve an 8-hour efficiency of 574 results per 8 hours per m². In certain embodiments, the methods and systems embodied herein can achieve, 2,225 results per 8 hours (including startup), and at a footprint of approximately 1.94 m² (e.g., approximately 1.32 m×1.47 m), would achieve an 8-hour efficiency of 1,147 results per 8 hours per m². Additionally, or alternatively, should the samples employed in the methods and systems embodied herein be multiplex, either via eluate splitting or via amplification of multiple target nucleic acids in a single amplification reaction, the 8-hour efficiency will increase proportionally. For example, but not by way of limitation, the detection of an additional target nucleic acid using a multiplex reaction can increase the 8-hour efficiency by about 574 results per 8 hours per m². In certain embodiments, if a multiplexing analysis for determining two target nucleic acids is performed in the absence of eluate splitting, the 8-hour efficiency can be about 1,148 results per 8 hours per m². In certain embodiments, if a multiplexing analysis for determining four target nucleic acids is performed in the absence of eluate splitting, the 8-hour efficiency can be about 2,296 results per 8 hours per m². In certain embodiments, if a multiplexing analysis for determining four target nucleic acids is performed in combination with eluate splitting, the 8-hour efficiency can be about 4,592 results per 8 hours per m².

Additionally, or alternatively, and in accordance with another aspect of the disclosed subject matter, the methods and systems of screening donor blood samples can perform a nucleic acid analysis on a plurality of samples to detect one or a plurality of pathogens or infectious agents, where determinations of a predetermined level of nucleic acids derived from each of the pathogens or infectious agents can be based on the nucleic acid analyses performed, and the methods and systems can perform screening to achieve at least about 70 results per hour per m³ or even at least about 140 results per hour per m² in accordance with the disclosed subject matter. In certain embodiments, the determinations can be indicative of whether the donor blood can be released for clinical use. Additionally, the methods and systems can determine whether the donor blood is acceptable for transfusion based in part on the nucleic acid analysis result.

For example, but not by way of limitation, the methods of the present disclosure can achieve an efficiency of at least about 15 results per hour per m³. In certain embodiments, the methods of the present disclosure can achieve an efficiency of at least about 20 results per hour per m³. In certain embodiments, the methods and systems of the present disclosure can achieve an efficiency of at least about 25 results per hour per m³. In certain embodiments, the methods of the present disclosure can achieve an efficiency of at least about 30 results per hour per m³. In certain embodiments, the methods of the present disclosure can achieve an efficiency of at least about 35 results per hour per m³. In certain embodiments, the methods of the present disclosure can achieve an efficiency of at least about 40 results per hour per m³. In certain embodiments, the methods of the present disclosure can achieve an efficiency of at least about 45 results per hour per m³. In certain embodiments, the methods of the present disclosure can achieve an efficiency of at least about 50 results per hour per m³. In certain embodiments, the methods of the present disclosure can achieve an efficiency of at least about 55 results per hour per m³. In certain embodiments, the methods of the present disclosure can achieve an efficiency of at least about 60 results per hour per m³. In certain embodiments, the methods of the present disclosure can achieve an efficiency of at least about 65 results per hour per m³. In certain embodiments, the methods of the present disclosure can achieve an efficiency of at least about 70 results per hour per m³. In certain embodiments, the methods of the present disclosure can achieve an efficiency of at least about 75 results per hour per m³. In certain embodiments, the methods of the present disclosure can achieve an efficiency of at least about 80 results per hour per m³. In certain embodiments, the methods of the present disclosure can achieve an efficiency of at least about 85 results per m³. In certain embodiments, the methods of the present disclosure can achieve an efficiency of at least about 90 results per hour per m³. In certain embodiments, the methods of the present disclosure can achieve an efficiency of at least about 95 results per hour per m³. In certain embodiments, the methods of the present disclosure can achieve an efficiency of at least about 100 results per hour per m³. In certain embodiments, the methods of the present disclosure can achieve an efficiency of at least about 105 results per hour per m³. In certain embodiments, the methods of the present disclosure can achieve an efficiency of at least about 110 results per hour per m³. In certain embodiments, the methods of the present disclosure can achieve an efficiency of at least about 115 results per hour per m³. In certain embodiments, the methods of the present disclosure can achieve an efficiency of at least about 120 results per hour per m³. In certain embodiments, the methods and systems of the present disclosure can achieve an efficiency of at least about 125 results per hour per m³. In certain embodiments, the methods of the present disclosure can achieve an efficiency of at least about 130 results per hour per m³. In certain embodiments, the methods of the present disclosure can achieve an efficiency of at least about 135 results per hour per m³. In certain embodiments, the methods of the present disclosure can achieve an efficiency of at least about 140 results per hour per m³. In certain embodiments, the methods of the present disclosure can achieve an efficiency of at least about 145 results per hour per m³. In certain embodiments, the methods of the present disclosure can achieve an efficiency of at least about 150 results per hour per m³. In certain embodiments, the methods of the present disclosure can achieve an efficiency of at least about 155 results per hour per m³. In certain embodiments, the methods of the present disclosure can achieve an efficiency of at least about 160 results per hour per m³. In certain embodiments, the methods of the present disclosure can achieve an efficiency of at least about 165 results per hour per m³. In certain embodiments, the methods of the present disclosure can achieve an efficiency of at least about 170 results per hour per m³. In certain embodiments, the methods of the present disclosure can achieve an efficiency of at least about 175 results per hour per m³. In certain embodiments, the methods of the present disclosure can achieve an efficiency of at least about 180 results per hour per m³. In certain embodiments, the methods of the present disclosure can achieve an efficiency of at least about 185 results per hour per m³. In certain embodiments, the methods of the present disclosure can achieve an efficiency of at least about 190 results per hour per m³. In certain embodiments, the methods of the present disclosure can achieve an efficiency of at least about 195 results per hour per m³. In certain embodiments, the methods of the present disclosure can achieve an efficiency of at least about 200 results per hour per m³. In certain embodiments, the methods of the present disclosure can achieve an efficiency of at least about 205 results per hour per m³. In certain embodiments, the methods of the present disclosure can achieve an efficiency of at least about 210 results per hour per m³. In certain embodiments, the methods of the present disclosure can achieve an efficiency of at least about 215 results per hour per m³. In certain embodiments, the methods of the present disclosure can achieve an efficiency of at least about 220 results per hour per m³. In certain embodiments, the methods and systems of the present disclosure can achieve an efficiency of at least about 225 results per hour per m³. In certain embodiments, the methods of the present disclosure can achieve an efficiency of at least about 230 results per hour per m³. In certain embodiments, the methods of the present disclosure can achieve an efficiency of at least about 235 results per hour per m³. In certain embodiments, the methods of the present disclosure can achieve an efficiency of at least about 240 results per hour per m³. In certain embodiments, the methods of the present disclosure can achieve an efficiency of at least about 245 results per hour per m³. In certain embodiments, the methods of the present disclosure can achieve an efficiency of at least about 250 results per hour per m³. In certain embodiments, the methods of the present disclosure can achieve an efficiency of at least about 255 results per hour per m³. In certain embodiments, the methods of the present disclosure can achieve an efficiency of at least about 260 results per hour per m³. In certain embodiments, the methods of the present disclosure can achieve an efficiency of at least about 265 results per hour per m³. In certain embodiments, the methods of the present disclosure can achieve an efficiency of at least about 270 results per hour per m³. In certain embodiments, the methods of the present disclosure can achieve an efficiency of at least about 275 results per hour per m³. In certain embodiments, the methods of the present disclosure can achieve an efficiency of at least about 280 results per hour per m³. In certain embodiments, the methods of the present disclosure can achieve an efficiency of at least about 285 results per hour per m³. In certain embodiments, the methods of the present disclosure can achieve an efficiency of at least about 290 results per hour per m³. In certain embodiments, the methods of the present disclosure can achieve an efficiency of at least about 295 results per hour per m³. In certain embodiments, the methods of the present disclosure can achieve an efficiency of at least about 300 results per hour per m³.

In certain embodiments, the methods of the present disclosure can achieve an efficiency of at least about 30 results per hour per m². In certain embodiments, the methods of the present disclosure can achieve an efficiency of at least about 35 results per hour per m². In certain embodiments, the methods of the present disclosure can achieve an efficiency of at least about 40 results per hour per m². In certain embodiments, the methods of the present disclosure can achieve an efficiency of at least about 45 results per hour per m². In certain embodiments, the methods of the present disclosure can achieve an efficiency of at least about 50 results per hour per m². In certain embodiments, the methods of the present disclosure can achieve an efficiency of at least about 55 results per hour per m². In certain embodiments, the methods of the present disclosure can achieve an efficiency of at least about 60 results per hour per m². In certain embodiments, the methods of the present disclosure can achieve an efficiency of at least about 65 results per hour per m². In certain embodiments, the methods of the present disclosure can achieve an efficiency of at least about 70 results per hour per m². In certain embodiments, the methods of the present disclosure can achieve an efficiency of at least about 75 results per hour per m². In certain embodiments, the methods of the present disclosure can achieve an efficiency of at least about 80 results per hour per m². In certain embodiments, the methods of the present disclosure can achieve an efficiency of at least about 85 results per hour per m². In certain embodiments, the methods of the present disclosure can achieve an efficiency of at least about 90 results per hour per m². In certain embodiments, the methods of the present disclosure can achieve an efficiency of at least about 95 results per hour per m². In certain embodiments, the methods of the present disclosure can achieve an efficiency of at least about 100 results per hour per m². In certain embodiments, the methods of the present disclosure can achieve an efficiency of at least about 105 results per hour per m². In certain embodiments, the methods of the present disclosure can achieve an efficiency of at least about 110 results per hour per m². In certain embodiments, the methods of the present disclosure can achieve an efficiency of at least about 115 results per hour per m². In certain embodiments, the methods of the present disclosure can achieve an efficiency of at least about 120 results per hour per m². In certain embodiments, the methods of the present disclosure can achieve an efficiency of at least about 125 results per hour per m². In certain embodiments, the methods of the present disclosure can achieve an efficiency of at least about 130 results per hour per m². In certain embodiments, the methods of the present disclosure can achieve an efficiency of at least about 135 results per hour per m². In certain embodiments, the methods of the present disclosure can achieve an efficiency of at least about 140 results per hour per m². In certain embodiments, the methods of the present disclosure can achieve an efficiency of at least about 145 results per hour per m². In certain embodiments, the methods of the present disclosure can achieve an efficiency of at least about 150 results per hour per m². In certain embodiments, the methods of the present disclosure can achieve an efficiency of at least about 155 results per hour per m². In certain embodiments, the methods of the present disclosure can achieve an efficiency of at least about 160 results per hour per m². In certain embodiments, the methods of the present disclosure can achieve an efficiency of at least about 165 results per hour per m². In certain embodiments, the methods of the present disclosure can achieve an efficiency of at least about 170 results per hour per m². In certain embodiments, the methods of the present disclosure can achieve an efficiency of at least about 175 results per hour per m². In certain embodiments, the methods of the present disclosure can achieve an efficiency of at least about 180 results per hour per m². In certain embodiments, the methods of the present disclosure can achieve an efficiency of at least about 185 results per hour per m². In certain embodiments, the methods of the present disclosure can achieve an efficiency of at least about 190 results per hour per m². In certain embodiments, the methods of the present disclosure can achieve an efficiency of at least about 195 results per hour per m². In certain embodiments, the methods of the present disclosure can achieve an efficiency of at least about 200 results per hour per m². In certain embodiments, the methods of the present disclosure can achieve an efficiency of at least about 205 results per hour per m². In certain embodiments, the methods of the present disclosure can achieve an efficiency of at least about 210 results per hour per m². In certain embodiments, the methods of the present disclosure can achieve an efficiency of at least about 215 results per hour per m². In certain embodiments, the methods of the present disclosure can achieve an efficiency of at least about 220 results per hour per m². In certain embodiments, the methods of the present disclosure can achieve an efficiency of at least about 225 results per hour per m². In certain embodiments, the methods of the present disclosure can achieve an efficiency of at least about 230 results per hour per m². In certain embodiments, the methods of the present disclosure can achieve an efficiency of at least about 235 results per hour per m². In certain embodiments, the methods of the present disclosure can achieve an efficiency of at least about 240 results per hour per m². In certain embodiments, the methods of the present disclosure can achieve an efficiency of at least about 245 results per hour per m². In certain embodiments, the methods of the present disclosure can achieve an efficiency of at least about 250 results per hour per m². In certain embodiments, the methods of the present disclosure can achieve an efficiency of at least about 255 results per hour per m². In certain embodiments, the methods of the present disclosure can achieve an efficiency of at least about 260 results per hour per m². In certain embodiments, the methods of the present disclosure can achieve an efficiency of at least about 265 results per hour per m². In certain embodiments, the methods of the present disclosure can achieve an efficiency of at least about 270 results per hour per m². In certain embodiments, the methods of the present disclosure can achieve an efficiency of at least about 275 results per hour per m². In certain embodiments, the methods of the present disclosure can achieve an efficiency of at least about 280 results per hour per m². In certain embodiments, the methods of the present disclosure can achieve an efficiency of at least about 285 results per hour per m². In certain embodiments, the methods of the present disclosure can achieve an efficiency of at least about 290 results per hour per m². In certain embodiments, the methods of the present disclosure can achieve an efficiency of at least about 295 results per hour per m². In certain embodiments, the methods of the present disclosure can achieve an efficiency of at least about 300 results per hour per m². In certain embodiments, the methods of the present disclosure can achieve an efficiency of at least about 310 results per hour per m². In certain embodiments, the methods of the present disclosure can achieve an efficiency of at least about 320 results per hour per m². In certain embodiments, the methods of the present disclosure can achieve an efficiency of at least about 330 results per hour per m². In certain embodiments, the methods of the present disclosure can achieve an efficiency of at least about 340 results per hour per m². In certain embodiments, the methods of the present disclosure can achieve an efficiency of at least about 350 results per hour per m². In certain embodiments, the methods of the present disclosure can achieve an efficiency of at least about 360 results per hour per m². In certain embodiments, the methods of the present disclosure can achieve an efficiency of at least about 370 results per hour per m². In certain embodiments, the methods of the present disclosure can achieve an efficiency of at least about 380 results per hour per m². In certain embodiments, the methods of the present disclosure can achieve an efficiency of at least about 380 results per hour per m². In certain embodiments, the methods of the present disclosure can achieve an efficiency of at least about 400 results per hour per m². In certain embodiments, the methods of the present disclosure can achieve an efficiency of at least about 410 results per hour per m². In certain embodiments, the methods of the present disclosure can achieve an efficiency of at least about 420 results per hour per m². In certain embodiments, the methods of the present disclosure can achieve an efficiency of at least about 430 results per hour per m². In certain embodiments, the methods of the present disclosure can achieve an efficiency of at least about 440 results per hour per m². In certain embodiments, the methods of the present disclosure can achieve an efficiency of at least about 450 results per hour per m². In certain embodiments, the methods of the present disclosure can achieve an efficiency of at least about 460 results per hour per m². In certain embodiments, the methods of the present disclosure can achieve an efficiency of at least about 470 results per hour per m². In certain embodiments, the methods of the present disclosure can achieve an efficiency of at least about 480 results per hour per m². In certain embodiments, the methods of the present disclosure can achieve an efficiency of at least about 490 results per hour per m². In certain embodiments, the methods of the present disclosure can achieve an efficiency of at least about 500 results per hour per m². In certain embodiments, the methods of the present disclosure can achieve an efficiency of at least about 510 results per hour per m². In certain embodiments, the methods of the present disclosure can achieve an efficiency of at least about 520 results per hour per m². In certain embodiments, the methods of the present disclosure can achieve an efficiency of at least about 530 results per hour per m². In certain embodiments, the methods of the present disclosure can achieve an efficiency of at least about 540 results per hour per m². In certain embodiments, the methods of the present disclosure can achieve an efficiency of at least about 550 results per hour per m². In certain embodiments, the methods of the present disclosure can achieve an efficiency of at least about 560 results per hour per m². In certain embodiments, the methods of the present disclosure can achieve an efficiency of at least about 570 results per hour per m². In certain embodiments, the methods of the present disclosure can achieve an efficiency of at least about 580 results per hour per m². In certain embodiments, the methods of the present disclosure can achieve an efficiency of at least about 590 results per hour per m². In certain embodiments, the methods of the present disclosure can achieve an efficiency of at least about 600 results per hour per m².

### 5.3 Release Methods Incorporating Specific Process Steps

In accordance with another aspect of the disclosed subject matter, the methods and systems of the present disclosure for donor blood or donor material release comprise performance of a nucleic acid analysis on the sample of donor blood to detect a plurality of pathogens or infectious agents wherein the nucleic acid analysis comprises a specific process step. A “specific process step” as used herein, refers to a particularly identified strategy relating to one or more of sample preparation, amplification, or detection, as well as particular sample processing strategies, such as those that allow for the prioritization of a sample or of the detection of a particular pathogen or infectious agent.

Additionally, or alternatively, and in accordance with another aspect of the disclosed subject matter, the methods and systems of screening can be used with pooled donor blood samples to perform a nucleic acid analysis on a pooled sample to detect one or a plurality of pathogens or infectious agents. A determination of a predetermined level of nucleic acids derived from each of the pathogens or infectious agents can be based on the nucleic acid analysis performed. Upon a determination of the presence of a nucleic acid derived from at least one of the pathogens or infectious agents at or in excess of the predetermined level in the pooled sample, the methods and systems can screen samples of individual donor blood or sub-pools of donor blood included in the pooled sample, by a nucleic acid analysis of the samples to detect one or a plurality of pathogens or infectious agents. Determinations of a predetermined level of nucleic acids derived from each of the pathogens or infectious agents can be based on the nucleic acid analyses performed. The determinations can be indicative of whether the donor blood can be released for clinical use. Additionally, the methods and systems can determine whether the donor blood is acceptable for transfusion based in part on the nucleic acid analysis result. In certain embodiments, the methods and systems can determine whether a material derived from the donor of the blood sample is acceptable for transfusion based in part on the nucleic acid analysis result.

Additionally, or alternatively, and in accordance with another aspect of the disclosed subject matter, the methods and systems of screening can be used with pooled donor blood samples to perform a nucleic acid analysis on a pooled sample to detect one or a plurality of pathogens or infectious agents. A determination of a presence or absence of nucleic acids derived from each of the plurality of pathogens or infectious agents based on the nucleic acid analysis of the pooled sample can be indicative of whether donor blood and/or donor material can be released for clinical use. Additionally, the methods and systems can determine whether donor blood is acceptable for transfusion based in part on the nucleic acid analysis result.

Additionally, or alternatively, and in accordance with another aspect of the disclosed subject matter, upon a determination of the presence of a nucleic acid derived from at least one of a plurality of pathogens or infectious agents in a pooled sample, the methods and systems of screening can further include nucleic acid analysis of individual samples or sub-pools thereof included in the pooled sample and making a determination whether donor blood and/or donor material associated with individual samples or sub-pools thereof can be released for clinical use. For example, the systems and methods can determine whether donor blood associated with individual samples or sub-pools thereof is acceptable for transfusion based at least in part on the nucleic acid analysis of the individual samples or sub-pools thereof.

Additionally, or alternatively, the methods and systems for rapid NAT screening embodied herein provide specific advantages in deconstructing pooled samples. While the advantages are particularly pronounced in connection with the extensive pooling associated with screening of plasma, e.g., where pooling of 512 individual plasma samples is currently available, the advantages of the methods and systems of the instant disclosure are significant even with smaller pools, e.g., pools routinely employed in the context of screening donor blood other than plasma. Additionally, or alternatively, the methods and systems for rapid NAT screening embodied herein can pool whole blood samples, or serum or plasma samples using the same onboard sample processing hardware used for individual samples, as described further herein. For purpose of example and not limitation, the methods and systems for rapid NAT screening embodied herein can pool 6 lysed whole blood samples or 24 serum or plasma samples. The following table, Table 2, compares the time associated with current deconstruction strategies for exemplary pool sizes with the time associated with deconstruction strategies employing the methods and systems describe herein (the times provided are for screening only). With respect to the deconstruction paths illustrated, the bold samples are the pools where a pathogen or infectious agent has been identified warranting further deconstruction. The deconstruction methods described in Table 2 are for purpose of example and illustration only and not limitation. Additional or alternative deconstruction algorithms and methods can be used depending on the desired performance of the system and application. The Conventional Systems and Rapid NAT systems referenced in Table 2 incorporate liquid handlers or poolers to deconstruct the sample pools. The times provided for deconstruction with Conventional Systems in Table 2 refer to the instrument testing time to screen the deconstructed pools and exclude additional deconstruction time required for preparation of the deconstruction pools and/or manual transport of the deconstruction pools. The additional deconstruction time for preparation of the deconstruction pools can depend on the size of the deconstruction pools used. For purpose of example and not limitation, the additional deconstruction time for preparation of the deconstruction pools can range from several minutes up to an hour or more depending on the size of the pool to be deconstructed and the size of the deconstruction pools used. The times provided in Table 2 for deconstruction with Rapid NAT are inclusive of the time required to prepare the deconstruction pools.

TABLE 2

| No. of | | | |
|---|---|---|---|
| Samples | Conventional | Conventional | |
| Pooled | Deconstruction | Systems | Rapid NAT |
| 8 | 8 | 3.5 hrs | 0.6 hr |
| | 4 × 4 | 3.5 hrs | 0.6 hr |
| | 2 × 2 | 3.5 hrs | 0.6 hr |
| | 1 × 1 | 3.5 hrs | 0.6 hr |
| | | Total: 14 hrs | Total: 2.4 hrs |
| 96 | 96 | 3.5 hrs | 0.6 hr |
| | 48 × 48 | 3.5 hrs | 0.6 hr |
| | 24 × 24 | 3.5 hrs | 0.6 hr |
| | 12 × 12 | 3.5 hrs | 0.6 hr |
| | 6 × 6 | 3.5 hrs | 0.6 hr |
| | 3 × 3 | 3.5 hrs | 0.6 hr |
| | 1 × 1 × 1 | 3.5 hrs | 0.6 hr |
| | | Total: 24.5 hrs | Total: 4.2 hrs |
| 512 | 512 | 3.5 hrs | 0.6 hr |
| | 256 × 256 | 3.5 hrs | 0.6 hr |
| | 128 × 128 | 3.5 hrs | 0.6 hr |
| | 64 × 64 | 3.5 hrs | 0.6 hr |
| | 32 × 32 | 3.5 hrs | 0.6 hr |
| | 16 × 16 | 3.5 hrs | 0.6 hr |
| | 8 × 8 | 3.5 hrs | 0.6 hr |
| | | 3.5 hrs | 0.6 hr |
| | 4 × 4 | 3.5 hrs | 0.6 hr |
| | 2 × 2 | 3.5 hrs | 0.6 hr |
| | 1 × 1 | Total: 35 hrs | Total: 6 hrs |

In certain embodiments, a positive pooled sample can be immediately deconstructed into eight individual donor samples. In such situations, the conventional systems would require, for screening alone, at least 7 hours (3.5 hours for the initial sample and then approximately 3.5 hours for the individual samples). In contrast, the methods and systems would require, for screening alone, about 1.2 hours (0.6 hours for the initial sample and then approximately 0.6 hours for the individual samples). Additionally or alternatively, a positive pooled sample can be deconstructed into other pool sizes, such as into two pools of 6 individual donor samples.

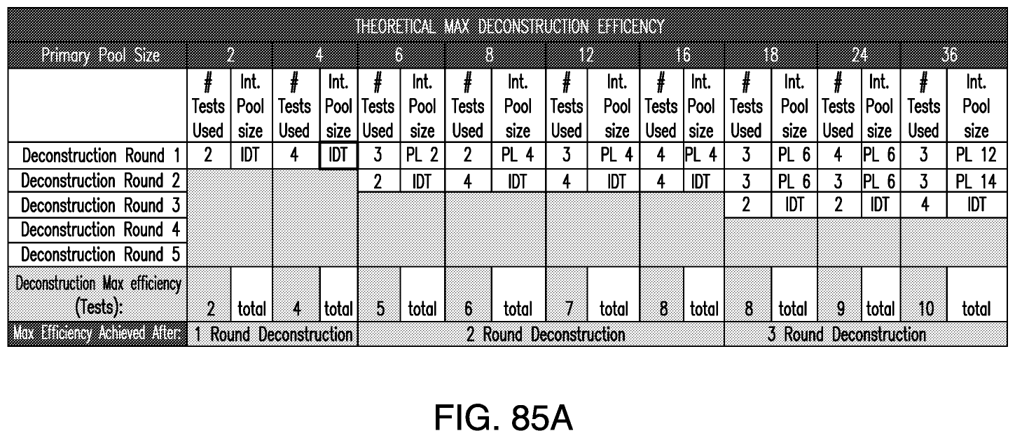

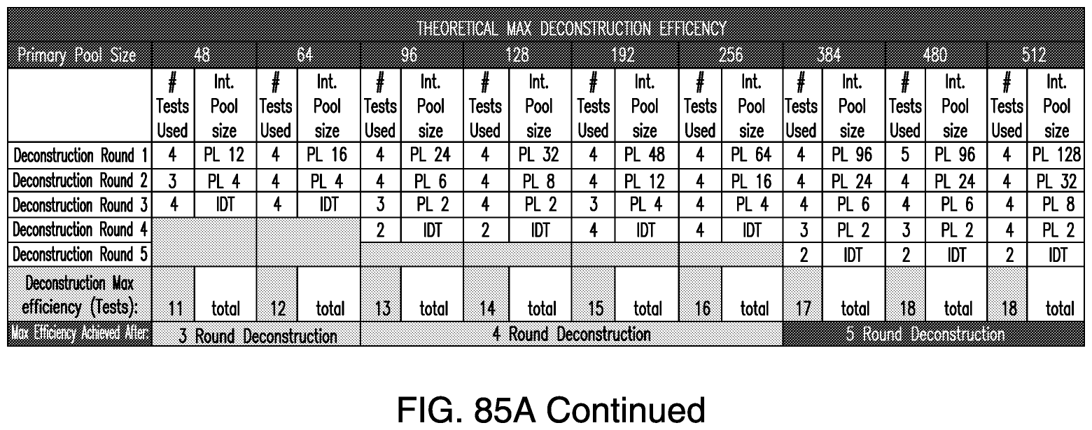

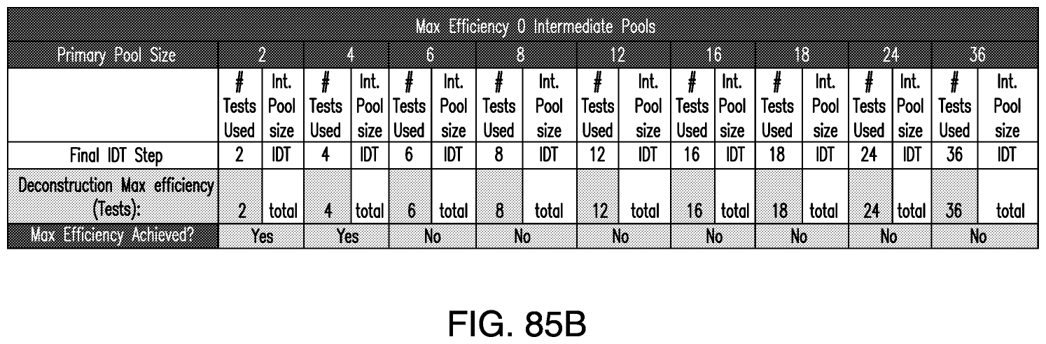

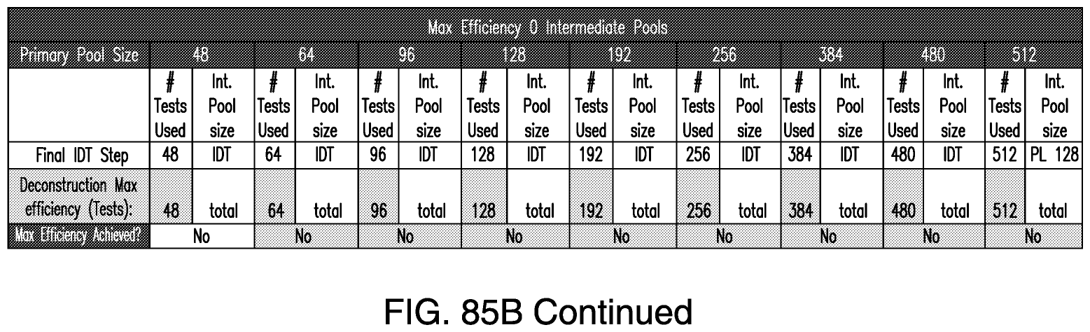

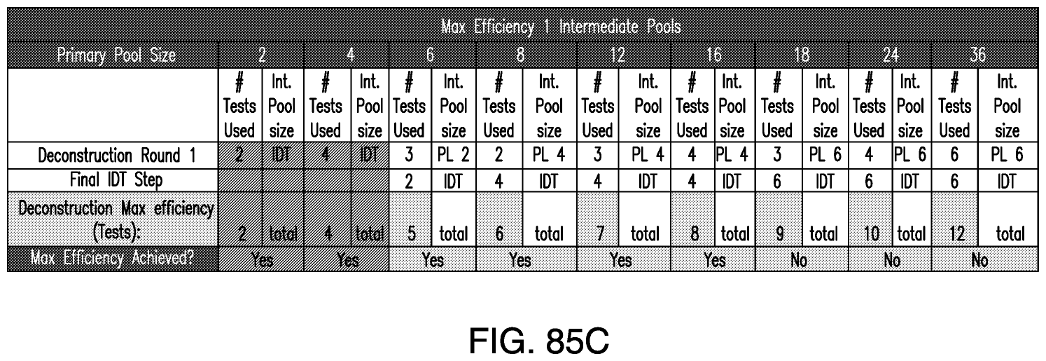

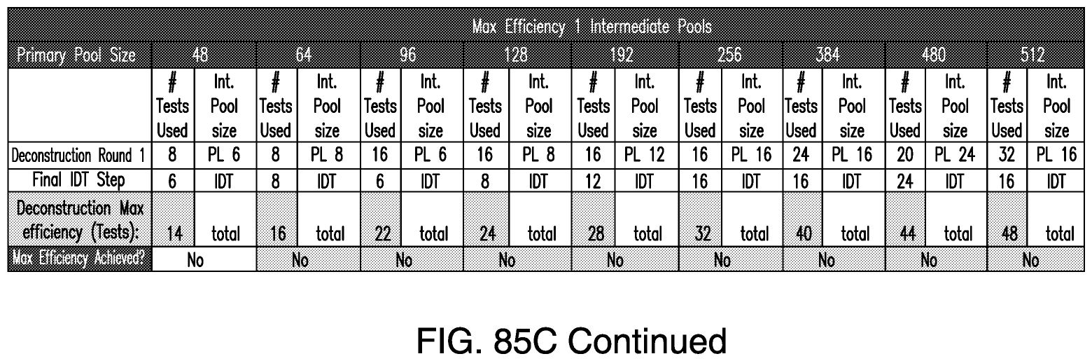

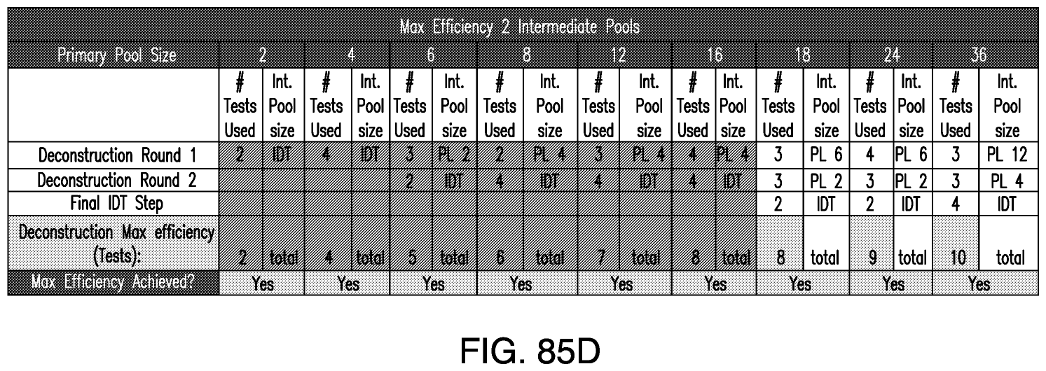

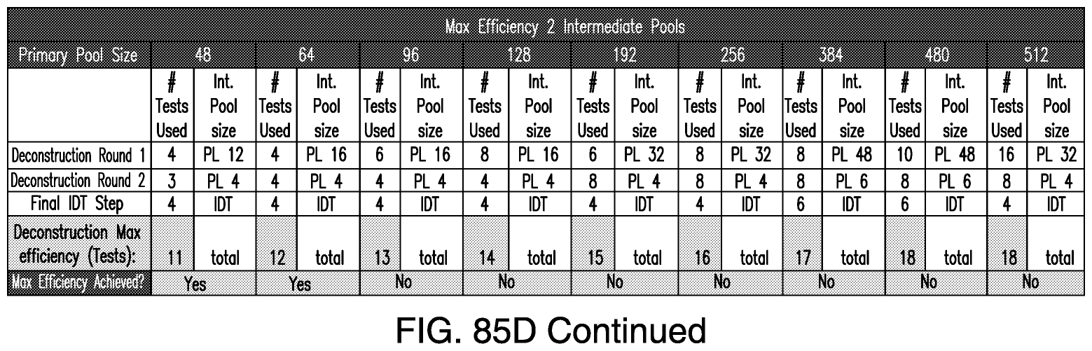

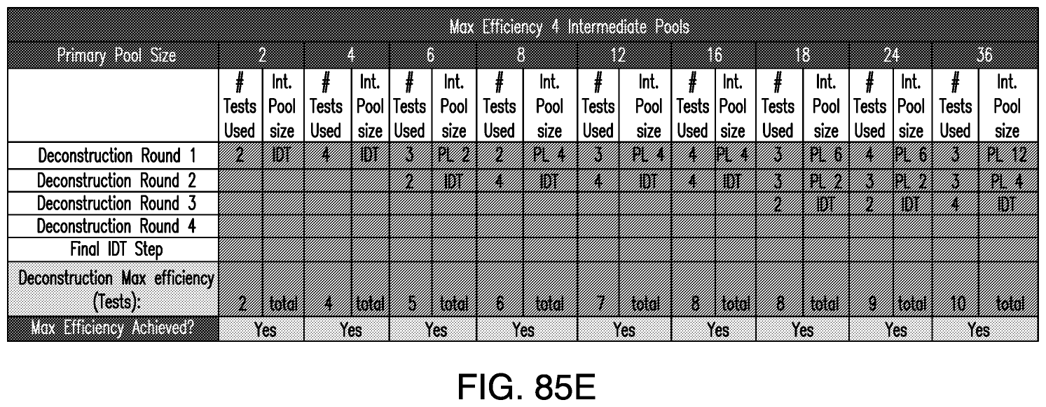

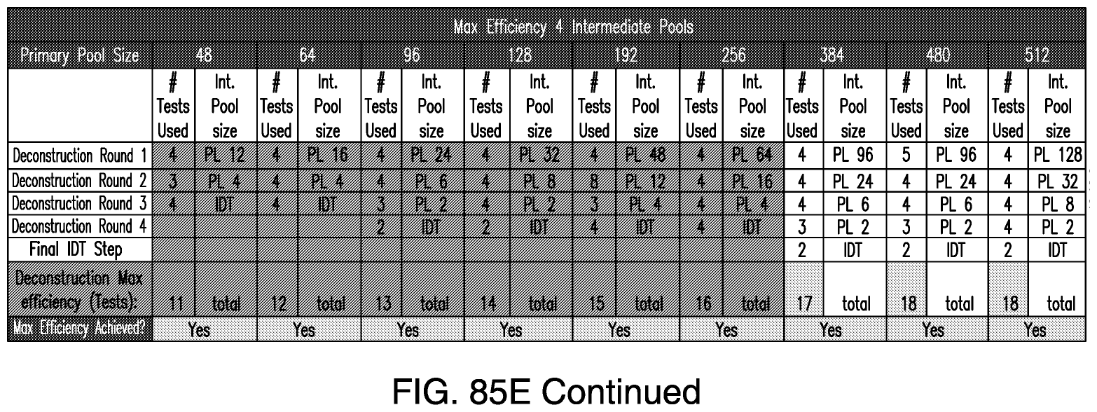

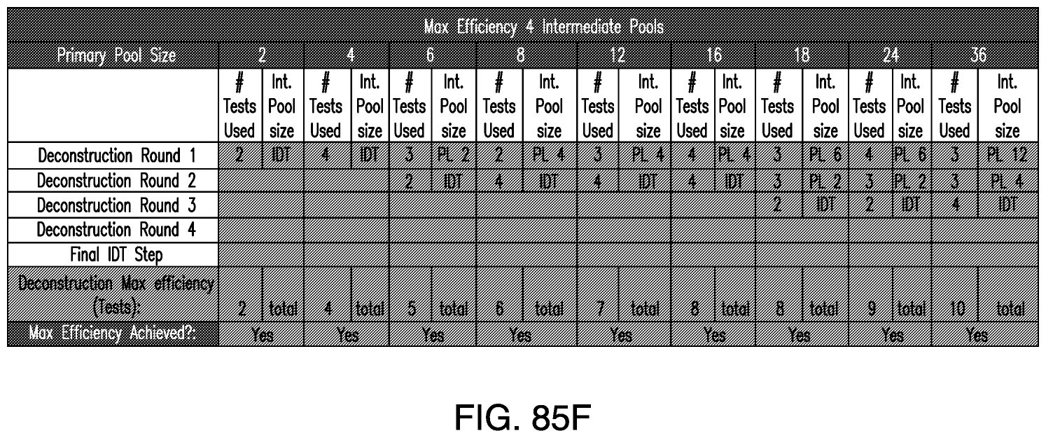

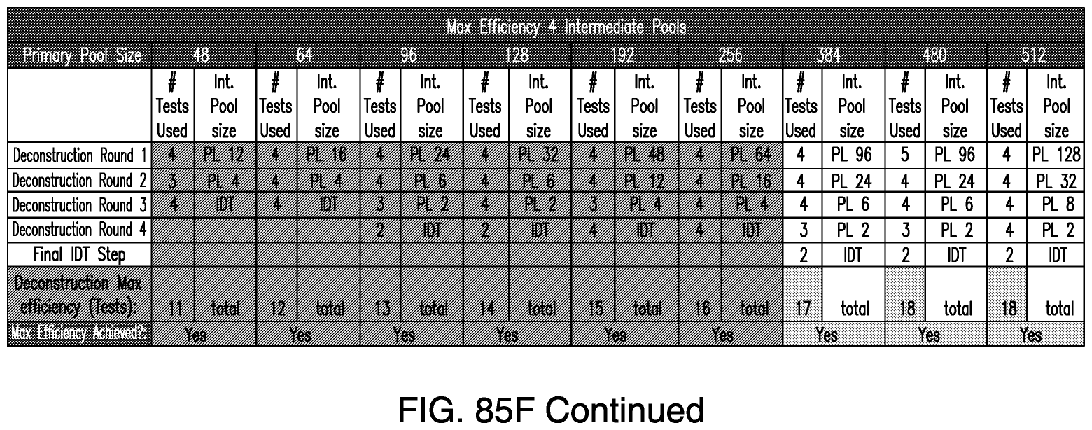

For purpose of example and illustration, additional exemplary pool deconstruction techniques are shown in FIGS. 85A-85F. With reference to FIG. 85A, as pool size increases, pool deconstruction efficiency can be improved by using one or more rounds of sub-pool testing to deconstruct the pooled sample. For example, and as shown in FIG. 85A, using one or more rounds of sub-pool testing can reduce the number of tests needed to deconstruct a sample. For example, a pooled sample of 24 can be deconstructed using three rounds of deconstruction testing and 9 total tests. Alternatively, a pooled sample of 24 can be deconstructed using two rounds of deconstruction testing (i.e., one round of sub-pool testing followed by individual donor sample testing, or IDT testing) and 10 total tests. Additionally or alternatively, a pooled sample of 24 can be deconstructed using only individual donor sample testing, i.e., only IDT testing, and 24 total tests.

Although using one or more rounds of sub-pool testing can improve deconstruction efficiency, conventional deconstruction methods often incorporate only a single round of sub-pool testing, or no sub-pool testing. For example, pooling samples to form a pooled sample or a sub-pool of the pooled sample conventionally can be performed on a dedicated liquid handler. Forming sub-pools for deconstruction of a pooled sample can include manually retrieving and transferring the constituent samples used to form the pooled sample to a liquid handler to be pooled into sub-pools and then manually transferring the sub-pools to an instrument for analysis. In conventional deconstruction, the process of manually transferring constituent samples to and from a liquid handler can then be repeated to form additional sub-pools for additional rounds of deconstruction testing. The additional time and complexity associated with using a dedicated liquid handler to form sub-pools for deconstruction can outweigh the benefits of using additional rounds of deconstruction testing, and as such, conventional deconstruction methods can be limited to a single round of sub-pool testing. Additionally or alternatively, conventional deconstruction testing can include only IDT testing for deconstruction.

Automated systems and methods for onboard pooling as embodied herein can increase deconstruction efficiency. For purpose of example and as embodied herein, the constituent samples used to form a pooled sample can be stored at the sample loading area during nucleic acid analysis of the pooled sample, and upon a determination that the pooled sample is reactive, the system can automatically initiate further onboard pooling and nucleic acid analysis of the constituent samples to deconstruct the pooled sample. Additionally or alternatively, sub-pool size and the number of rounds of sub-pool testing can be selected to maximize deconstruction efficiency.

For example, but not limitation, a pooled sample of donor blood, e.g., whole blood, lysed whole blood, serum, or plasma, as embodied herein, comprises blood from two donors. In certain embodiments, the pooled sample of donor blood comprises blood from three donors. In certain embodiments, the pooled sample of donor blood comprises blood from four donors. In certain embodiments, the pooled sample of donor blood comprises blood from five donors. In certain embodiments, the pooled sample of donor blood comprises blood from six donors. In certain embodiments, the pooled sample of donor blood comprises blood from seven donors. In certain embodiments, the pooled sample of donor blood comprises blood from eight donors. In certain embodiments, the pooled sample of donor blood comprises blood from nine donors. In certain embodiments, the pooled sample of donor blood comprises blood from 10 donors. In certain embodiments, the pooled sample of donor blood comprises blood from 11 donors. In certain embodiments, the pooled sample of donor blood comprises blood from 12 donors. In certain embodiments, the pooled sample of donor blood comprises blood from 13 donors. In certain embodiments, the pooled sample of donor blood comprises blood from 14 donors. In certain embodiments, the pooled sample of donor blood comprises blood from 15 donors. In certain embodiments, the pooled sample of donor blood comprises blood from 16 donors. In certain embodiments, the pooled sample of donor blood comprises blood from 17 donors. In certain embodiments, the pooled sample of donor blood comprises blood from 18 donors. In certain embodiments, the pooled sample of donor blood comprises blood from 19 donors. In certain embodiments, the pooled sample of donor blood comprises blood from 20 donors. In certain embodiments, the pooled sample of donor blood comprises blood from 21 donors. In certain embodiments, the pooled sample of donor blood comprises blood from 22 donors. In certain embodiments, the pooled sample of donor blood comprises blood from 23 donors. In certain embodiments, the pooled sample of donor blood comprises blood from 24 donors. In certain embodiments, the pooled sample of donor blood comprises blood from 25 donors. In certain embodiments, the pooled sample of donor blood comprises blood from 26 donors. In certain embodiments, the pooled sample of donor blood comprises blood from 27 donors. In certain embodiments, the pooled sample of donor blood comprises blood from 28 donors. In certain embodiments, the pooled sample of donor blood comprises blood from 29 donors. In certain embodiments, the pooled sample of donor blood comprises blood from 30 donors. In certain embodiments, the pooled sample of donor blood comprises blood from 31 donors. In certain embodiments, the pooled sample of donor blood comprises blood from 32 donors. In certain embodiments, the pooled sample of donor blood comprises blood from 33 donors. In certain embodiments, the pooled sample of donor blood comprises blood from 34 donors. In certain embodiments, the pooled sample of donor blood comprises blood from 35 donors. In certain embodiments, the pooled sample of donor blood comprises blood from 36 donors. In certain embodiments, the pooled sample of donor blood comprises blood from 37 donors. In certain embodiments, the pooled sample of donor blood comprises blood from 38 donors. In certain embodiments, the pooled sample of donor blood comprises blood from 39 donors. In certain embodiments, the pooled sample of donor blood comprises blood from 40 donors. In certain embodiments, the pooled sample of donor blood comprises blood from 41 donors. In certain embodiments, the pooled sample of donor blood comprises blood from 42 donors. In certain embodiments, the pooled sample of donor blood comprises blood from 43 donors. In certain embodiments, the pooled sample of donor blood comprises blood from 44 donors. In certain embodiments, the pooled sample of donor blood comprises blood from 45 donors. In certain embodiments, the pooled sample of donor blood comprises blood from 46 donors. In certain embodiments, the pooled sample of donor blood comprises blood from 47 donors. In certain embodiments, the pooled sample of donor blood comprises blood from 48 donors. In certain embodiments, the pooled sample of donor blood comprises blood from 49 donors. In certain embodiments, the pooled sample of donor blood comprises blood from 50 donors. In certain embodiments, the pooled sample of donor blood comprises blood from 51 donors. In certain embodiments, the pooled sample of donor blood comprises blood from 52 donors. In certain embodiments, the pooled sample of donor blood comprises blood from 53 donors. In certain embodiments, the pooled sample of donor blood comprises blood from 54 donors. In certain embodiments, the pooled sample of donor blood comprises blood from 55 donors. In certain embodiments, the pooled sample of donor blood comprises blood from 56 donors. In certain embodiments, the pooled sample of donor blood comprises blood from 57 donors. In certain embodiments, the pooled sample of donor blood comprises blood from 58 donors. In certain embodiments, the pooled sample of donor blood comprises blood from 59 donors. In certain embodiments, the pooled sample of donor blood comprises blood from 60 donors. In certain embodiments, the pooled sample of donor blood comprises blood from 61 donors. In certain embodiments, the pooled sample of donor blood comprises blood from 62 donors. In certain embodiments, the pooled sample of donor blood comprises blood from 63 donors. In certain embodiments, the pooled sample of donor blood comprises blood from 64 donors. In certain embodiments, the pooled sample of donor blood comprises blood from 65 donors. In certain embodiments, the pooled sample of donor blood comprises blood from 66 donors. In certain embodiments, the pooled sample of donor blood comprises blood from 67 donors. In certain embodiments, the pooled sample of donor blood comprises blood from 68 donors. In certain embodiments, the pooled sample of donor blood comprises blood from 69 donors. In certain embodiments, the pooled sample of donor blood comprises blood from 70 donors. In certain embodiments, the pooled sample of donor blood comprises blood from 71 donors. In certain embodiments, the pooled sample of donor blood comprises blood from 72 donors. In certain embodiments, the pooled sample of donor blood comprises blood from 73 donors. In certain embodiments, the pooled sample of donor blood comprises blood from 74 donors. In certain embodiments, the pooled sample of donor blood comprises blood from 75 donors. In certain embodiments, the pooled sample of donor blood comprises blood from 76 donors. In certain embodiments, the pooled sample of donor blood comprises blood from 77 donors. In certain embodiments, the pooled sample of donor blood comprises blood from 78 donors. In certain embodiments, the pooled sample of donor blood comprises blood from 79 donors. In certain embodiments, the pooled sample of donor blood comprises blood from 80 donors. In certain embodiments, the pooled sample of donor blood comprises blood from 81 donors. In certain embodiments, the pooled sample of donor blood comprises blood from 82 donors. In certain embodiments, the pooled sample of donor blood comprises blood from 83 donors. In certain embodiments, the pooled sample of donor blood comprises blood from 84 donors. In certain embodiments, the pooled sample of donor blood comprises blood from 85 donors. In certain embodiments, the pooled sample of donor blood comprises blood from 86 donors. In certain embodiments, the pooled sample of donor blood comprises blood from 87 donors. In certain embodiments, the pooled sample of donor blood comprises blood from 88 donors. In certain embodiments, the pooled sample of donor blood comprises blood from 89 donors. In certain embodiments, the pooled sample of donor blood comprises blood from 90 donors. In certain embodiments, the pooled sample of donor blood comprises blood from 91 donors. In certain embodiments, the pooled sample of donor blood comprises blood from 92 donors. In certain embodiments, the pooled sample of donor blood comprises blood from 93 donors. In certain embodiments, the pooled sample of donor blood comprises blood from 94 donors. In certain embodiments, the pooled sample of donor blood comprises blood from 95 donors. In certain embodiments, the pooled sample of donor blood comprises blood from 96 donors. In certain embodiments, the pooled sample of donor blood comprises blood from 97 donors. In certain embodiments, the pooled sample of donor blood comprises blood from 98 donors. In certain embodiments, the pooled sample of donor blood comprises blood from 99 donors. In certain embodiments, the pooled sample of donor blood comprises blood from 100 donors. In certain embodiments, the pooled sample of donor blood comprises blood from 2-1,000 donors. In certain embodiments, the pooled sample of donor blood comprises blood from 2-500 donors. In certain embodiments, the pooled sample of donor blood comprises blood from 2-250 donors. In certain embodiments, the pooled sample of donor blood comprises blood from 2-150 donors. In certain embodiments, the pooled sample of donor blood comprises blood from 2-100 donors. In certain embodiments, the pooled sample of donor blood comprises blood from 25-100 donors. In certain embodiments, the pooled sample of donor blood comprises blood from 50-100 donors. In certain embodiments, the pooled sample of donor blood comprises blood from 75-100 donors. In certain embodiments, the pooled sample of donor blood comprises blood from more than 100 donors.

In certain embodiments, the pooled sample of donor blood comprises plasma from 2-10,000 donors. In certain embodiments, the pooled sample of donor blood comprises plasma from 2-9,000 donors. In certain embodiments, the pooled sample of donor blood comprises plasma from 2-8,000 donors. In certain embodiments, the pooled sample of donor blood comprises plasma from 2-7,000 donors. In certain embodiments, the pooled sample of donor blood comprises plasma from 2-6,000 donors. In certain embodiments, the pooled sample of donor blood comprises plasma from 2-5,000 donors. In certain embodiments, the pooled sample of donor blood comprises plasma from 2-4,000 donors. In certain embodiments, the pooled sample of donor blood comprises plasma from 2-3,000 donors. In certain embodiments, the pooled sample of donor blood comprises plasma from 2-2,500 donors. In certain embodiments, the pooled sample of donor blood comprises plasma from 2-2,000 donors. In certain embodiments, the pooled sample of donor blood comprises plasma from 2-1,500 donors. In certain embodiments, the pooled sample of donor blood comprises plasma from 2-1,000 donors. In certain embodiments, the pooled sample of donor blood comprises plasma from 2-900 donors. In certain embodiments, the pooled sample of donor blood comprises plasma from 2-800 donors. In certain embodiments, the pooled sample of donor blood comprises plasma from 2-700 donors. In certain embodiments, the pooled sample of donor blood comprises plasma from 2-600 donors. In certain embodiments, the pooled sample of donor blood comprises plasma from 2-500 donors. In certain embodiments, the pooled sample of donor blood comprises plasma from 2-400 donors. In certain embodiments, the pooled sample of donor blood comprises plasma from 2-300 donors. In certain embodiments, the pooled sample of donor blood comprises plasma from 2-250 donors. In certain embodiments, the pooled sample of donor blood comprises plasma from 2-200 donors. In certain embodiments, the pooled sample of donor blood comprises plasma from 2-150 donors. In certain embodiments, the pooled sample of donor blood comprises plasma from 2-100 donors. In certain embodiments, the pooled sample of donor blood comprises plasma from 2-90 donors. In certain embodiments, the pooled sample of donor blood comprises plasma from 2-80 donors. In certain embodiments, the pooled sample of donor blood comprises plasma from 2-70 donors. In certain embodiments, the pooled sample of donor blood comprises plasma from 2-60 donors. In certain embodiments, the pooled sample of donor blood comprises plasma from 2-50 donors. In certain embodiments, the pooled sample of donor blood comprises plasma from 2-40 donors. In certain embodiments, the pooled sample of donor blood comprises plasma from 2-30 donors. In certain embodiments, the pooled sample of donor blood comprises plasma from 2-20 donors. In certain embodiments, the pooled sample of donor blood comprises plasma from 2-10 donors. In certain embodiments, the pooled sample of donor blood comprises plasma from 2-5 donors.

In certain embodiments, the methods and systems of the present disclosure for donor blood release or donor material release comprise performance of a nucleic acid analysis on the sample of donor blood to detect a plurality of pathogens or infectious agents wherein the nucleic acid analysis comprises a nucleic acid amplification reaction. For example, but not by way of limitation, the nucleic acid amplification reaction can be an isothermal reaction. In certain embodiments, the isothermal reaction is a recombinase polymerase amplification. In certain embodiments, the isothermal reaction is a nicking enzyme amplification reaction.

In certain embodiments, the methods of the present disclosure for donor blood release or donor material release comprise performance of a nucleic acid analysis on the sample of donor blood to detect a plurality of pathogens or infectious agents wherein the nucleic acid analysis comprises optical detection of the presence of a nucleic acid derived from at least one of the plurality of pathogens or infectious agents. In certain embodiments, the methods of the present disclosure for donor blood release comprise performance of a nucleic acid analysis on the sample of donor blood to detect a plurality of pathogens or infectious agents wherein the nucleic acid analysis comprises digital detection of the presence of a nucleic acid derived from at least one of the plurality of pathogens or infectious agents.

In certain embodiments, the methods of the present disclosure for donor blood release or donor material release comprise performance of a nucleic acid analysis on the sample of donor blood to detect a plurality of pathogens or infectious agents wherein the nucleic acid analysis comprises contemporaneous contact of the sample with sample lysis buffer and a protease.

In certain embodiments, the methods of the present disclosure for donor blood release or donor material release comprise performance of a nucleic acid analysis on the sample of donor blood to detect a plurality of pathogens or infectious agents wherein the nucleic acid analysis comprises contacting the sample with CuTi-coated microparticles.

In certain embodiments, the methods of the present disclosure for donor blood release or donor material release comprise performance of a nucleic acid analysis on the sample of donor blood to detect a plurality of pathogens or infectious agents wherein the nucleic acid analysis comprises contacting the sample with a plurality of microparticles and translation of the microparticles on a surface via magnetic force. In certain embodiments, the translation of the microparticles on a surface via magnetic force is provided by a moving permanent magnet and/or an electromagnet, e.g., a stationary electromagnet.

In certain embodiments, the methods of the present disclosure for donor blood release comprise performance of a nucleic acid analysis on the sample of donor blood to detect a plurality of pathogens or infectious agents wherein the nucleic acid analysis comprises: purification of nucleic acid from the sample of donor blood; division of the purified nucleic acid into a plurality of fractions; and at least one fraction is reserved for further screening

In certain embodiments, the methods of the present disclosure for donor blood release allow for “Stat” sample processing. “Stat” sample processing, as used herein, refers to the ability to prioritize a sample either already being processed in connection with a method of the present disclosure or to introduce a new sample that is prioritized over samples currently being processed in connection with a method of the present disclosure. For example, but not by way of limitation, the methods of the present disclosure for donor blood release comprise: performing a nucleic acid analysis on a plurality of samples of donor blood to detect a plurality of pathogens or infectious agents; wherein the order of the nucleic acid analysis of individual samples of the plurality of donor samples can be modified and wherein upon a determination of the absence of a predetermined level of nucleic acids derived from each of the plurality of pathogens or infectious agents based on the nucleic acid analysis, indicates release of the donor blood associated with the analyzed donor sample for clinical use. In certain embodiments, the methods of the present disclosure for donor blood release occur in the absence of immunoassay analysis of the donor blood.

### 5.4 Release Methods Comprising Screening for Specific Target Nucleic Acids

In accordance with another aspect of the disclosed subject matter, the methods and systems of the present disclosure for donor blood release or donor material release comprise performance of a nucleic acid analysis on the sample of donor blood to detect a plurality of pathogens or infectious agents. For example but not by way of limitation, the plurality of pathogens or infectious agents can be selected from the group consisting of SARS-CoV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, and HEV. In certain embodiments, the plurality of pathogens or infectious agents can be selected from the group consisting of: SARS-CoV-2 (COVID-19), coronaviruses, HIV-1, HIV-2, HBV, HCV, CMV, Epstein-Barr virus (EBV), human T-lymphotropic virus (HTLV) Parvo B19 Virus, HAV, syphilis, Chlamydia, Gonorrhea, Dengue, Chikungunya, WNV, HEV, Usutu Virus and Creutzfeldt-Jakob disease (vCJD).

In certain embodiments, the nucleic acid analysis is used to determine the presence or absence of one or more pathogens or infectious agents, e.g., in a sample, e.g., a sample of donor blood. In certain embodiments, the nucleic acid analysis is used to determine the level of one or more pathogens or infectious agents, e.g., in a sample, e.g., a sample of donor blood.

In certain embodiments, the methods of the present disclosure for donor blood release or donor material release comprise performance of a nucleic acid analysis on a sample of donor blood to detect a plurality of pathogens or infectious agents wherein the plurality of pathogens or infectious agents are HIV-1, HIV-2, HCV, and HBV. In certain embodiments, the plurality of pathogens or infectious agents are HIV-1, HIV-2, HCV, HBV, and WNV. In certain embodiments, the plurality of pathogens or infectious agents are HIV-1, HIV-2, HCV, HBV, WNV, and Zika Virus. In certain embodiments, the plurality of pathogens or infectious agents are HIV-1, HIV-2, HCV, HBV, WNV, Zika Virus, and Chikungunya Virus. In certain embodiments, the plurality of pathogens or infectious agents are HIV-1, HIV-2, HCV, HBV, WNV, Zika Virus, Chikungunya Virus, and Dengue Virus. In certain embodiments, the plurality of pathogens or infectious agents are HIV-1, HIV-2, HCV, HBV, WNV, Zika Virus, Chikungunya Virus, Dengue Virus, and Babesia. In certain embodiments, the plurality of pathogens or infectious agents are HIV-1, HIV-2, HCV, HBV, WNV, Zika Virus, Chikungunya Virus, Dengue Virus, Babesia, and Malaria. In certain embodiments, the plurality of pathogens or infectious agents are HIV-1, HIV-2, HCV, HBV, WNV, Zika Virus, Chikungunya Virus, Dengue Virus, Babesia, Malaria, and Parvovirus B19. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, and HAV. In certain embodiments, the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

In certain embodiments, the methods and systems of the present disclosure for donor blood release or donor material release comprise performance of a nucleic acid analysis on a sample of donor blood to detect a plurality of pathogens or infectious agents wherein the plurality of pathogens or infectious agents are Zika Virus and WNV. In certain embodiments, the methods of the present disclosure for donor blood release or donor material release comprise performance of a nucleic acid analysis on a sample of donor blood to detect a plurality of pathogens or infectious agents wherein the plurality of pathogens or infectious agents are Chikungunya Virus and Dengue Virus. In certain embodiments, the plurality of pathogens or infectious agents are Zika Virus, WNV, Chikungunya Virus and Dengue Virus. In certain embodiments, the plurality of pathogens or infectious agents are HIV-1, HIV-2, HCV, HBV, and Zika Virus. In certain embodiments, the plurality of pathogens or infectious agents are HIV-1, HIV-2, HCV, HBV, Zika Virus, and Dengue Virus. In certain embodiments, the plurality of pathogens or infectious agents are HIV-1, HIV-2, HCV, HBV, Zika Virus, Dengue Virus, and Chikungunya Virus. In certain embodiments, the plurality of pathogens or infectious agents are HIV-1, HIV-2, HCV, HBV, Zika Virus, Dengue Virus, and WNV. In certain embodiments, the plurality of pathogens or infectious agents are HIV-1, HIV-2, HCV, HBV, Zika Virus, WNV, and Chikungunya Virus. In certain embodiments, the plurality of pathogens or infectious agents are HIV-1, HIV-2, HCV, HBV, Zika Virus, WNV, Dengue Virus, and Chikungunya Virus. In certain embodiments, the plurality of pathogens or infectious agents are HIV-1, HIV-2, HCV, HBV, and Dengue Virus. In certain embodiments, the plurality of pathogens or infectious agents are HIV-1, HIV-2, HCV, HBV, Dengue Virus, and Chikungunya Virus. In certain embodiments, the plurality of pathogens or infectious agents are HIV-1, HIV-2, HCV, HBV, Dengue Virus, WNV, and Chikungunya Virus. In certain embodiments, the plurality of pathogens or infectious agents are HIV-1, HIV-2, HCV, HBV, and Chikungunya Virus. In certain embodiments, the plurality of pathogens or infectious agents are HIV-1, HIV-2, HCV, HBV, Chikungunya Virus, and Zika Virus. In certain embodiments, the plurality of pathogens or infectious agents are HIV-1, HIV-2, HCV, HBV, Chikungunya Virus, Zika Virus, and WNV. In certain embodiments, the plurality of pathogens or infectious agents are HIV-1, HIV-2, HCV, HBV, WNV, and Dengue Virus. In certain embodiments, the plurality of pathogens or infectious agents are HIV-1, HIV-2, HCV, HBV, Chikungunya Virus, Zika Virus, and WNV.

In certain embodiments, the methods of the present disclosure for donor blood release or donor material release comprise performance of a nucleic acid analysis on a sample of donor blood to detect a plurality of pathogens or infectious agents wherein the plurality of pathogens or infectious agents are Babesia and Malaria. In certain embodiments, the plurality of pathogens or infectious agents are HIV-1, HIV-2, HCV, HBV, and Babesia. In certain embodiments, the plurality of pathogens or infectious agents are HIV-1, HIV-2, HCV, HBV, and Malaria. In certain embodiments, the plurality of pathogens or infectious agents are HIV-1, HIV-2, HCV, HBV, Malaria, and Babesia.

In certain embodiments, the methods of the present disclosure for donor blood release or donor material release comprise performance of a nucleic acid analysis on a sample of donor blood to detect a plurality of pathogens or infectious agents wherein the plurality of pathogens or infectious agents are Parvovirus B19 and HAV. In certain embodiments, the nucleic acid analysis to detect Parvovirus B19 is a quantitative nucleic acid analysis. In certain embodiments, the plurality of pathogens or infectious agents are HIV-1, HIV-2, HCV, HBV, and Parvovirus B19. In certain embodiments, the plurality of pathogens or infectious agents are HIV-1, HIV-2, HCV, HBV, Parvovirus B19, and HAV.

In certain embodiments, the methods of the present disclosure for donor blood release or donor material release comprise performance of a nucleic acid analysis on a sample of donor blood to detect a plurality of pathogens or infectious agents wherein the plurality of pathogens or infectious agents are HIV-1, HIV-2, HCV, HBV, and HAV.

In certain embodiments, the methods of the present disclosure for donor blood release or donor material release comprise performance of a nucleic acid analysis on a sample of donor blood to detect a plurality of pathogens or infectious agents wherein the plurality of pathogens or infectious agents are HIV-1, HIV-2, HCV, HBV, and HEV.

### 5.5 Release Methods Incorporating Multiplex Strategies

In accordance with another aspect of the disclosed subject matter, the methods and systems of the present disclosure for donor blood release or donor material release comprise detecting one or a plurality of pathogens or infectious agents at predetermined levels, wherein the nucleic acid analysis comprises multiplex analysis. As used herein, “multiplex analysis” refers to concurrent screening for two or more target nucleic acids, e.g., where each target nucleic acid is derived from a pathogen or infectious agent. As used herein, “multiplex analysis” encompasses concurrent screening of two or more target nucleic acids in a single reaction vessel, e.g., an amplification vessel, as well as screening in separate reaction vessels of two or more target nucleic acids, e.g., where a sample eluate has been split into two more separate reaction vessels, e.g., amplification vessels, as described herein. In certain embodiments, the multiplex strategies described herein allow for, at least in part, the substantial improvements to time to result and efficiency e.g., number of results per hour per unit size, of the systems implementing the methods described herein.

In certain embodiments, the methods and systems of the present disclosure for donor blood release or donor material release comprise detecting plurality of pathogens or infectious agents at predetermined levels, wherein the plurality of pathogens or infectious agents are HIV-1, HIV-2, HCV, and HBV; and the nucleic acid analysis comprises multiplex analysis of HIV-1 and HIV-2. In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; and the nucleic acid analysis comprises multiplex analysis of HIV-1, HIV-2, and HCV. In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; and the nucleic acid analysis comprises multiplex analysis of HIV-1, HIV-2, and HBV. In certain embodiments, the methods of the present disclosure for donor blood release comprise detecting plurality of pathogens or infectious agents at predetermined levels, wherein the plurality of pathogens or infectious agents are HIV-1, HIV-2, HCV, and HBV; and the nucleic acid analysis comprises multiplex analysis of HCV, and HBV. In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV. In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; and the nucleic acid analysis comprises multiplex analysis of HIV-1, HIV-2, HCV, and HBV.

In certain embodiments, the methods and systems of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; and the nucleic acid analysis comprises multiplex analysis of Zika Virus and WNV. In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; and the nucleic acid analysis comprises multiplex analysis of Chikungunya Virus and Dengue Virus. In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus. In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus. In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

In certain embodiments, the methods of the present disclosure for donor blood release or donor material release comprise detecting plurality of pathogens or infectious agents at predetermined levels, wherein the plurality of pathogens or infectious agents comprise Babesia and Malaria; and the nucleic acid analysis comprises multiplex analysis of Babesia and Malaria.

In certain embodiments, the methods of the present disclosure for donor blood release or donor material release comprise detecting plurality of pathogens or infectious agents at predetermined levels, wherein the plurality of pathogens or infectious agents comprise Parvovirus B19 and HAV; and the nucleic acid analysis comprises multiplex analysis of Parvovirus B19 and HAV. In certain embodiments, the nucleic acid analysis to detect Parvovirus B19 is a quantitative nucleic acid analysis.

### 5.6 Release Methods Based on Predetermined Levels of Target Nucleic Acid

In accordance with another aspect of the disclosed subject matter, the sample preparation, target amplification, and detection strategies described herein allow for, at least in part, the substantial improvements to time to result and throughput per unit size of the systems implementing the methods described herein, to allow for donor blood screening at predetermined levels of target nucleic acid relevant to effectively surveil such donor blood. For example, but not by way of limitation, such predetermined levels can be those identified by guidance provided by governmental agencies or non-governmental organizations.

In certain embodiments, the methods and systems of the present disclosure for donor blood release or donor material release comprise detecting plurality of pathogens or infectious agents at predetermined levels, wherein the plurality of pathogens or infectious agents and predetermined levels are selected from the following: SARS-CoV-2 (COVID-19) at a predetermined level of at least 1-50 copies/mL; HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; CMV at a predetermined level of at least 10-50 IU/mL; Parvovirus B19 at a predetermined level of at least 1-40 IU/mL; HAV at a predetermined level of at least 1-10 IU/mL; Chlamydia at a predetermined level of at least 100-500 copies/mL; Gonorrhea at a predetermined level of at least 100-500 copies/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Influenza at a predetermined level of at least 10-500 copies/mL; Babesia at a predetermined level of at least 1-20 copies/mL; Malaria at a predetermined level of at least 1-50 copies/mL; and HEV at a predetermined level of at least 1-20 IU/mL.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

In certain embodiments, the methods of the present disclosure for donor blood release or donor material release comprise detecting plurality of pathogens or infectious agents at predetermined levels, wherein the plurality of pathogens or infectious agents are Zika Virus and WNV; and wherein the predetermined levels are: at least 1-50 copies/mL of Zika Virus; and at least 1-50 copies/mL of WNV.

In certain embodiments, the methods of the present disclosure for donor blood release or donor material release comprise detecting plurality of pathogens or infectious agents at predetermined levels, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents are Babesia and Malaria; and wherein the predetermined levels are: at least 1-20 copies/mL of Babesia; and at least 1-50 copies/mL of Malaria.

In certain embodiments, the methods of the present disclosure for donor blood release or donor material release comprise detecting plurality of pathogens or infectious agents at predetermined levels, wherein the plurality of pathogens or infectious agents are Parvovirus B19 and HAV; and wherein the predetermined levels are: at least 1-40 IU/mL of Parvovirus B19; and at least 1-10 IU/mL of HAV. In certain embodiments, the nucleic acid analysis to detect Parvovirus B19 is a quantitative nucleic acid analysis

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

In certain embodiments, the methods of the present disclosure for donor blood release or donor material release comprise detecting plurality of pathogens or infectious agents at predetermined levels, wherein the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

In certain embodiments, the methods of the present disclosure for donor blood release or donor material release comprise detecting plurality of pathogens or infectious agents at predetermined levels, wherein the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

In certain embodiments, the methods of the present disclosure for donor blood release comprise detecting plurality of pathogens or infectious agents at predetermined levels, wherein the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

In certain embodiments, determining a level of the pathogen or infectious agent at or above a predetermined level is equivalent to detecting the presence of the pathogen or infectious agent, e.g., in the sample, e.g., blood sample. In certain embodiments, determining a level of the pathogen or infectious agent below a predetermined level is equivalent to determining the absence of the pathogen or infectious agent, e.g., in the sample, e.g., blood sample.

### 6. Analytical Systems

Reference will now be made in detail to various exemplary systems and components thereof of the disclosed subject matter, which are illustrated in the accompanying drawings and described in greater detail below. Each of aspects as described above, e.g., sample preparation aspects, amplification aspects, and detection aspects, of the disclosed subject matter have various benefits and it is recognized the various aspects and corresponding methods and performance parameters as previously described in detail can be selectively combined to achieve the desired benefits of the methods and systems disclosed herein. As such, the versatility and flexibility of the different aspects of the methods and resulting benefits contemplated by the disclosed subject matter will be further understood in conjunction with the various systems and components that can be uniquely combined as contemplated by the disclosure herein.

For example, and in accordance with another aspect of the disclosed subject matter, the methods and systems of screening donor blood samples can include a sample analysis station and a processor and memory with instructions to be executed. The sample analysis station can include a sample loading area, a sample preparation area, a nucleic acid amplification area, and a nucleic acid detection area. When the instructions are executed by the processor, the system can perform a nucleic acid analysis on a sample of donor blood to detect one or a plurality of pathogens or infectious agents. Upon a determination of a predetermined level of nucleic acids derived from each of the plurality of pathogens or infectious agents based on the nucleic acid analysis, the methods and systems are indicative of release of the donor blood or a donor material for clinical use. Additionally, the methods and systems can determine whether the donor blood is acceptable for transfusion based in part on the nucleic acid analysis result. As embodied herein, the screening of a plurality of donor blood samples for release of the donor blood or a donor material for clinical use and the nucleic acid analysis are performed independently, i.e., without a requirement that the plurality of samples be screened in a predetermined order prior to contact with the sample lysis buffer or that the specific nucleic acid analysis be predetermined prior to contact with the sample lysis buffer.

Additionally, or alternatively, and as embodied herein, the systems described herein can either comprise or interface with additional systems to communicate the results necessary to affect such release. For example, but not limitation, the systems described herein can comprise or interface with an informatics system, such as a Blood Establishment Computer System (BECS). Such BECS systems comprises software designed track donor information, donor blood screening results, and reduce the inadvertent transmission of disease in humans by limiting the release of blood and blood components to only those that have been shown to meet specific release criteria, e.g., negative results for one or more NAT-based assays.

Additionally, or alternatively, and as embodied herein, while certain aspects of the systems (e.g., the use of sample tube, reaction vessels, robotic pipettors, and various types of magnets) are described as acting in concert with each other, one of skill would recognize that various suitable options exist for the various aspects of the systems and the present disclosure contemplates systems that incorporate the substitution of such aspects. For example, but not by way of limitation, in certain embodiments, one or more sample preparation, amplification, and/or detection steps can be accomplished using robotic pipettors for fluid transfer and the introduction of reagents, while in certain embodiments, the use of magnets can substitute for one or more of the robotic pipettor-mediated steps. Similarly, in certain embodiments, sample preparation, amplification, and/or detection can occur in a stationary tube, well, or other vessel, while in other embodiments, one or more of sample preparation, amplification, and/or detection can occur in tube, well, or other vessel that moves, e.g., on tracks, from a first to a second (third, fourth, fifth, or more) position within a system.

Systems in accordance with the disclosed subject matter can include an analysis station, and the analysis station can include a sample loading area, a sample preparation area, a nucleic acid amplification area, and a nucleic acid detection area. For example and not limitation, a sample loading area can be configured to receive and store one or more sample tubes. For example and not limitation, sample tubes can be included in racks, and the sample loading area can receive one or more racks of sample tubes. As described further herein, a portion of a sample contained in a sample tube can be aspirated from the sample tube, such as for example, by a sample pipettor, and transferred within the system for nucleic acid analysis. For example and not limitation, a portion of a sample contained in a sample tube can be aspirated from the sample tube and transferred to a sample preparation area. Aspiration of a sample or portion thereof from a sample tube for nucleic acid analysis can occur at any suitable position within the system For purpose of example aspiration can occur at a location within the sample loading area. Additionally or alternatively, aspiration can occur at a sample aspiration position. For example and not limitation, a rack of sample tubes can be transferred from the loading area to an aspiration position within the system, e.g., with a robot, such as for example a robotic sample handler.

As embodied herein, sample tubes can remain on the system, e.g., in the sample loading area, while nucleic acid analysis is performed on the portion of the sample aspirated for nucleic acid analysis. As described further herein, the time to result performance of systems and methods in accordance with the disclosed subject matter can facilitate storing samples on the system, e.g., in the loading area, while nucleic acid analysis is performed. Storing sample tubes on the system while nucleic acid is performed on a portion of the sample can have advantages, such as for example and not limitation for onboard pool deconstruction, as described further herein.

Systems in accordance with the disclosed subject matter can further include a sample preparation area. In certain embodiments, samples can be transferred to the sample preparation area for a sample preparation process following initial aspiration of the sample for nucleic acid analysis. For example and not limitation, the sample preparation area can include a sample transport and a wash and elution system. For example and as embodied herein, the sample preparation area can include a sample a sample transport configured to transport one or more samples in a vessel along a transport path from a sample dispense position to a sample capture and transfer position. For example and as embodied herein, the sample transport can include a sample preparation carousel, e.g., a lysis carousel. For example and as embodied herein, exemplary sample preparation processes can include dispensing a sample into a vessel at the sample dispense position of the sample transport. For example and not limitation, a sample can be dispensed using a pipettor. Exemplary sample preparation processes can further include transporting the sample in a vessel along the transport path of the sample transport to the sample capture and transfer position. One or more reagents, such as for example lysis buffer and/or microparticles such as CuTi-coated microparticles, can be included in the vessel with the sample, depending on the desired sample preparation process. For example and not limitation, and as described further herein, exemplary sample preparation processes can include performing a lysis process, pre-treatment process, and/or onboard pooling process on the sample transport of the sample preparation area. As described further herein, exemplary pre-treatment and onboard pooling processes can include transferring samples between vessels on the sample transport as the vessels are transported along the transport path. For example, and as embodied herein, the sample preparation area can include a pipettor and the pipettor can transfer, e.g., aspirate and dispense, samples from and into vessels on the sample transport.

Exemplary systems can also include one or more particle transfer mechanisms. For example, systems can include a particle transfer mechanism to transfer microparticles, such as CuTi-coated microparticles bound with nucleic acids from the sample transport to the wash and elution system. In certain embodiments, the wash and elution system can include one or more wash vessels. For example, and as embodied herein, the wash and elution system can include a wash track having a plurality of wash vessels. The wash and elution system can perform a wash process, as described further herein. For example, and not limitation, the wash and elution system can include magnets to move microparticles within a wash vessel.

Systems in accordance with the disclosed subject matter can also include an amplification area and a detection area. For purpose of example and as embodied herein the amplification area and detection area can comprise an amplification and detection system. The amplification and detection system can include, for example, a carousel having one or more amplification vessels, and one or more detectors. As embodied herein, a sample can be transferred from the sample preparation area to the amplification and detection area for an amplification and detection process. For example, and not limitation, eluate from a wash process can be transferred, e.g., with a pipettor, to the amplification and detection system.

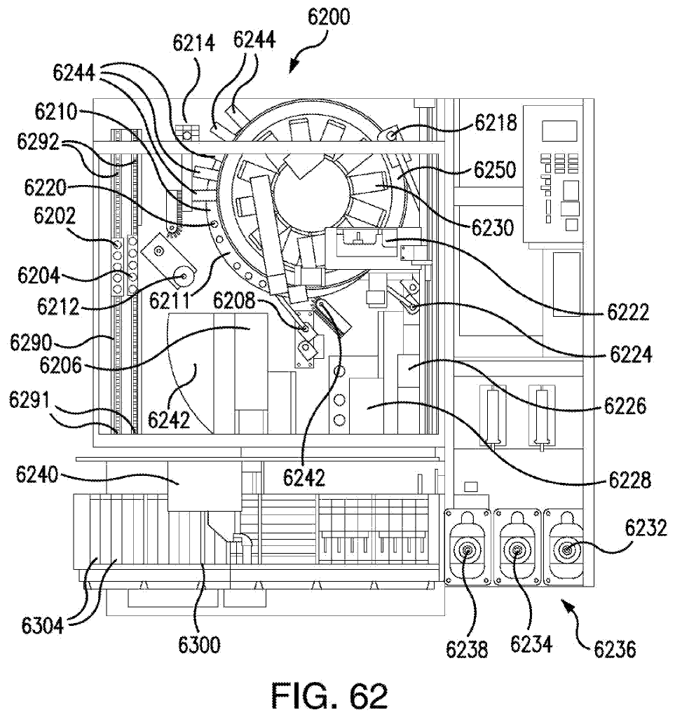

FIG. 62 depicts an exemplary system for practicing the inventions disclosed herein wherein the system of FIG. 62 can provide a NAT result in less than about 35 minutes. FIG. 62 is a plan view of an HTNAT sample analysis system 6200 for performing a sample preparation process, amplification process, and detection process, as well as additional components related to obtaining a result from a HTNAT sample analysis system, such as sample handling, consumable loading and waste disposal. FIG. 62 depicts a generally carousel-based system that comprises a sample preparation area having a sample transport 6210, and a wash and elution system 6230 and an amplification and detection system 6250. As embodied herein the sample transport can include a sample preparation carousel, e.g., rotating carousel 6211.

As exemplified in FIG. 62, a non-limiting embodiment of an HTNAT sample analysis system 6200 includes a sample tube 6204 within a rack of sample tubes 6202. A portion of the sample contained within the sample tube 6204 can be aspirated by the sample pipettor 6214 and transferred to the sample preparation area. The sample preparation area can include a sample transport, and as embodied herein, the sample transport can include a sample preparation carousel, e.g., rotating carousel 6211. As embodied herein, a portion of the sample contained within the sample tube 6204 can be aspirated by the sample pipettor 6214 and transferred to the sample transport 6210 in the sample preparation area, e.g., into a lysis tube 6220 positioned in a rotating carousel 6211. Consumables, such as lysis tube and lysis transfer tips can be stored within the system for use in the sample preparation area, e.g., at position 6242, for use in connection with the rotating carousel 6211. One or more loaders, such as for example, a lysis tube & lysis transfer tip loader 6208 can be employed to pick-and-place lysis tube and lysis transfer tips into the sample transport, e.g., rotating carousel 6211, In certain embodiments, the lysis tube can be filled with sample lysis buffer at the lysis reagent addition position 6212. Sample lysis buffer can be stored in one or more shelves or drawers, e.g., position 6238. Additionally, or alternatively after the sample lysis is complete, nucleic acids, e.g., nucleic acids bound to microparticles, can be transferred to a wash and elution system 6230, while the liquid remaining in the lysis tube is aspirated and disposed of as liquid waste and the lysis tube is disposed of as solid waste. The nucleic acids, e.g., nucleic acids bound to magnetic microparticles, can be transferred from a vessel on the sample transport, e.g., from a lysis tube on rotating carousel 6211 to the wash and elution system 6230 via a sample transfer mechanism, such as for example via collection in a lysis transfer tip, e.g., via positioning a magnet within the lysis transfer tip to facilitate the pick-and-place of the nucleic acids bound to magnetic microparticles into a wash vessel present on the wash and elution system 6230. Wash vessels can be stored on the system for use in the wash and elution system and can be transferred to the wash and elution system using one or more loaders. For example, wash vessels can be stored at position 6228 can be picked-and-placed via the wash/amp vessel loader 6224. As embodied herein the wash and elution system can include a track and the track can move wash vessels through the system. For example, and as embodied herein, the wash and elution system can include a track and wash vessels can be continuously transported along the track. For example, and as embodied herein, the wash and elution system can include a round track. As the wash vessels rotate around the wash and elution system 6230, the nucleic acids contained within the wash vessels can be mixed, e.g., using magnets as described in detail herein, and transferred from one wash vessel to another, e.g., using magnets as described herein. For example, a wash process can be performed on the wash and elution system 6230 as described herein, and the wash process can include one or more wash steps and an elution step. For example, upon transfer to an elution vessel, e.g., for an elution step, the nucleic acids can be contacted with an elution buffer. The elution vessel can be included in a wash vessel or can be separate vessel. As embodied herein, elution buffer can be stored on the system and can be transferred to a wash vessel or elution vessel, e.g., for an elution step. For example, and not limitation the elution buffer can be transferred using a pipettor. As embodied herein, elution buffer can be stored in a shelf or drawer 6234 and dispensed via a reagent pipettor 6222, and can be heated, e.g., via a resistive convention heater. In certain embodiments, after the nucleic acids are eluted from magnetic microparticles, e.g., in an elution step of a wash process, the magnetic microparticles can be removed from the elution vessel, e.g., by transferring the magnetic microparticles to a wash vessel, using magnets as described in detail herein. The eluate can then be transferred to an amplification and detection system, such as for example, with a pipettor. For example, and as embodied herein, eluate can be transferred from the elution vessel via an eluate pipettor 6218 to the amplification and detection system 6250. The 6250. As embodied herein, the amplification and detection system 6250 can include a rotatable carousel into which amplification vessels can be placed, e.g., amplification vessels stored at 6226 and transferred to the amplification and detection system 6230 by the wash/amp vessel loader 6224. As described in detail below, the addition of eluate, amplification reagents and activator can occur in a predetermined order, e.g., amplification reagents (“MasterMix”), followed by activator, followed by eluate, but any suitable order of addition is contemplated within the methods and systems described herein. The amplification reagents and activator can be dispensed into the amplification vessel via a pipettor, e.g., via the reagent pipettor 6222. Activator can be stored on a shelf or drawer, e.g., at position 6232. Rotation of the amplification vessel around the amplification and detection system 6230 not only allows suitable time for amplification but also positions the amplification vessels in communication with one or more detectors 6244 to facilitate detection.

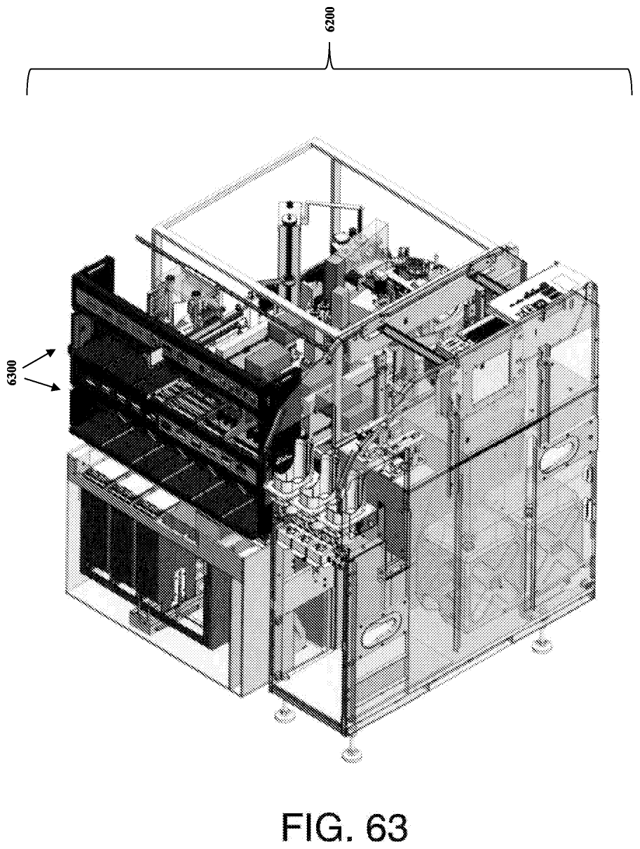

FIG. 63 is a perspective view of instrument 6200 of FIG. 62. In the exemplary embodiment of FIGS. 62-63 the sample analysis system 6200 can also contain additional subsystems that support testing of samples. Samples tubes 6204 in a sample tube rack 6202, may be loaded onto the system at a loading bay 6300 of a sample loading area. In one embodiment, the sample tube rack 6102 holds five sample tubes 6104. The loading bay 6300 comprises a plurality of locations 6304 for receiving sample tube racks 6102. Operators can load multiple sample tube racks 6104 onto the system for further processing. In the embodiment of FIGS. 62-63, the loading bay 6300 comprises two levels 6301 for receiving sample racks 6102 with each having 30 positions for receiving sample tube racks 6102. Where there are five sample tubes 6104 per sample tube rack 6102, the loading bay can receive up to 60 sample tube racks 6102 and up to 300 sample tubes 6104. The loading bay 6300 can have different configurations including having one or three or more levels to receive additional sample tube racks 6102.

The sample analysis system of FIGS. 62-63 may also have a robotic sample handler 6240 that transports the sample rack 6102 from the loading bay 6300 to another position within the sample loading area for further processing, such as aspiration a portion of the sample in the sample tube 6104 with a pipette. For example, as shown in FIG. 62, the robotic sample handler 6240 will pick up a sample tube rack 6102 from a position on the loading bay 6304 and place it onto a shuttle 6290 where a sample tube rack 6202 can be moved between the handoff position 6291 from the robotic sample handler 6240 and an aspiration position 6292 by the sample pipettor 6214. At that point, the sample undergoes the nucleic acid analysis process as described herein.

FIGS. 68A-68D depicts another exemplary system for practicing the disclosed subject matter described herein. FIG. 68A is a top view of an exemplary HTNAT sample analysis system 6800. FIG. 68B is a front view of the exemplary HTNAT sample analysis system. As embodied herein, the exemplary system 6800 has a footprint of about 1.47 m×about 1.27 m. The system 6800 includes an analysis station, and the analysis station includes a sample loading area, a sample preparation area, a nucleic acid amplification area, and a nucleic acid detection area. As described further herein, the configuration of the system (e.g., the configuration of the sample preparation area, nucleic acid amplification area, and a nucleic acid detection area) and the arrangements of the systems can contribute to the high levels of efficiency of the system. For example and as embodied herein, exemplary systems in accordance with the disclosed subject matter can provide a higher number of results per unit area of the system footprint as compared to traditional systems.

As illustrated in FIG. 68A, system 6800 includes, among other features disclosed below, consumable load bay. For example and not limitation, the system can use consumables for nucleic acid analysis, such as for example pipette tips, lysis tubes, wash vessels, and amplification vessels. As embodied herein consumables can be loaded into the system (e.g., in racks) for use in the system as described herein. For example and as depicted in FIG. 68B, the system can include a consumable load bay 6841 for eluate tips 6831, sample tips 6833, lysis vessels 6835, wash vessels 6837, and amplification vessels 6839. The consumables, e.g., the eluate tips 6831 sample tips 6833, lysis vessels 6835 wash vessels 6837, and amplification vessels 6839 can used in conjunction with the systems (e.g., sample preparation system and amplification and detection system) as described herein.

FIG. 68C is a plan view illustrating an exemplary analysis station including a sample preparation area, a nucleic acid amplification area, and a nucleic acid detection area for the system 6800 for performing sample preparation, amplification and detection, as well as additional components related to obtaining a result from a HTNAT sample analysis system, such as sample handling, consumable loading and waste disposal.

As embodied herein, the sample preparation area includes a sample transport 6805, a wash and elution system 6852, and a particle transfer mechanism 6803. As embodied herein the sample transport 6805 can include a lysis carousel. As described herein, the sample transport can transport vessels along a transport path from a sample dispense position to a sample capture and transfer position. As described herein, the sample transport, e.g., lysis carousel can be used to perform one or more sample preparation processes, such as for example a lysis process, pre-treatment process, and/or onboard pooling process. As embodied herein, the sample preparation area can also include at least one pipettor. For example and as embodied herein, the sample preparation area can include sample/reagent pipettor 6811. As described herein, the sample/reagent pipettor can, for example, perform an initial aspiration of a portion of a sample from a sample tube for nucleic acid analysis. For example and as embodied herein, sample/reagent pipettor 6811 can aspirate a portion of a sample from a sample tube at aspiration position 6892. As embodied herein, sample tubes can be transferred from the sample loading area 6842 and positioned at the sample aspiration position using one or more shuttles and/or robotic handlers as described further herein. As embodied herein, sample/reagent pipettor 6811 can also be used to pool samples on the sample transport 6805.

As described herein, sample preparation processes can include a lysis process on the sample transport, which can include contacting the sample with CuTi-coated microparticles. For purpose of illustration not limitation, a lysis process can include dispensing one or more samples into a vessel, e.g., lysis tube, on the sample transport (e.g., sample preparation carousel) 6805. As embodied herein, the sample can include serum or plasma samples and/or whole blood samples. On the lysis carousel 6805, the samples can be combined with lysis buffers, CuTi microparticles, internal control and optionally proteinase K proteins. As described further herein, the lysis process can disrupt the membranes or walls of pathogens, infectious agents, and/or cells present within a sample to release the nucleic acids present within the pathogens, infectious agents, and/or cells present in the sample. For purpose of illustration not limitation, the nucleic acid can be captured on the surface of the microparticles. As embodied herein, multiple aspects of the sample preparation process can be performed on the sample transport, e.g., sample preparation carousel 6805. For example, a lysis process can be performed. In certain embodiments, an onboard pooling process and a lysis process can be performed. In certain embodiments, a pre-treatment process, onboard pooling process and a lysis process can be performed.

After the desired sample preparation aspects are performed on the sample transport 6805, the microparticles can be transferred to the wash and elution system 6852. As embodied herein, the particle transfer mechanism 6803 connects the sample transport 6805 (e.g., lysis carousel or sample preparation carousel) and the wash and elution system 6852, which can reduce contamination in sample processing. The sample transfer mechanism can transfer microparticles using magnets, such as for example a magnetic tip, as described further herein. The wash and elution system can be used to perform additional sample preparation process aspects as described further herein. As embodied herein, the wash and elution system can include a wash track and can transport was vessels along the wash track. As embodied herein, the wash track of the wash and elution system 6852 can move continuously in a lock step fashion. For example, the wash and elution system 6852 can be used to perform a wash process as described further herein. As embodied herein, the wash process can include more than one wash steps, e.g., three washes steps, and can be performed on the wash track 6801 using wash vessels. For example and not limitation, wash processes can include moving microparticles within or between wells using magnets, e.g., external magnets. A first wash can be performed with lysis buffers to maintain the denaturing conditions for the nucleic acids. A second and third washes can be performed with water to remove the chemicals used during lysing. For example and as embodied herein, microparticles can be moved between wells for each wash step in each lock step, e.g., in about every 24 seconds. After the washes, an elution step can also be performed as part of the wash process. The elution step can include, for example, removing nucleic acid from the CuTi microparticles using heat to provide final eluate. For example, the elution step can also be performed on the wash and elution system 6852. For example and as embodied herein, the elution step can be performed in a wash vessel on the wash track.

For purpose of illustration not limitation, amplification and detection steps (e.g., an amplification and detection process) can be performed on the amplification and detection system, e.g., the amplification and detection subsystem 6807. As embodied herein, the amplification and detection subsystem 6807 can include one or more readers and an amplification and detection carousel having positions for amplification vessels. For example and not limitation and as embodied herein, the amplification and detection subsystem 6807 can include five independent fluorescent detectors. For purpose of example and as embodied herein, the amplification and detection carousel can rotate each amplification vessel past each of the five detectors with each lockstep, or about every 24 seconds, to produce detection signals on five independent fluorescent detectors on all samples in about every 24 seconds. For example and not limitation, amplification and detection can be performed in amplification vessels using magnesium as activator to mix with the purified nucleic acid from the samples. Additionally, or alternatively, isothermal incubation can be applied with the purified nucleic acid with simultaneous fluorescence detection, as described further herein.

As described above, nucleic acid analysis on automated systems such as exemplary system 6800 can consume consumables. For example and not limitation, consumables can include pipette tips, lysis tubes, wash vessels, and amplification vessels. Consumables can be packaged, such as in racks, and loaded onto the system. For example and as embodied herein, the system can include a loading area, such as for example loading area 6841. Consumables can be introduced into the system from the load bay for use in the nucleic acid analysis, e.g., by one or more robotic loaders. For example, amplification vessels 6839 can be transferred into the system from the loading area 6841, e.g., using a shuttle, and a amplification vessel loader 6824 can transfer the amplification vessels to the amplification and detection system, e.g., using pick and place techniques. Additionally or alternatively, lysis tips and/or lysis vessels 6835 can be transferred into the system from the loading area 6841, e.g., using a shuttle, and a lysis vessel and lysis tip loader 6808 can transfer lysis vessels, e.g., lysis tubes, to the sample transport 6805.

FIG. 68D is a plan view of pipettors above the processing deck illustrated in FIG. 68C. As shown, the system can incorporate one or more pipettor to move samples and/or reagents throughout the system. For example and as embodied herein, the system can include a sample/reagent pipettor 6811, an eluate transfer pipettor 6813, an amplification reagent pipettor 6815, and a sample preparation reagent pipettor 6817.

### 6.1 Sample Preparation Systems

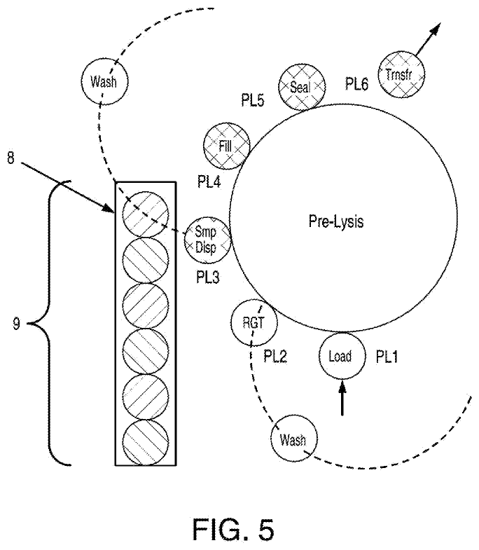

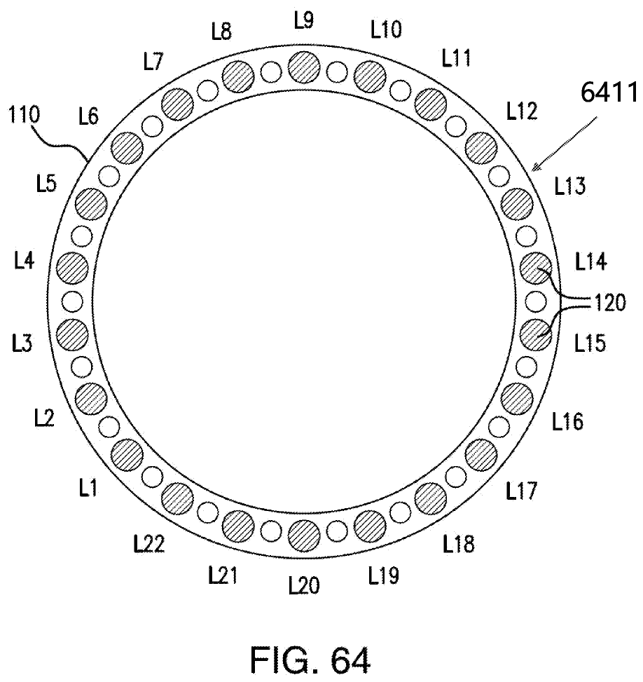

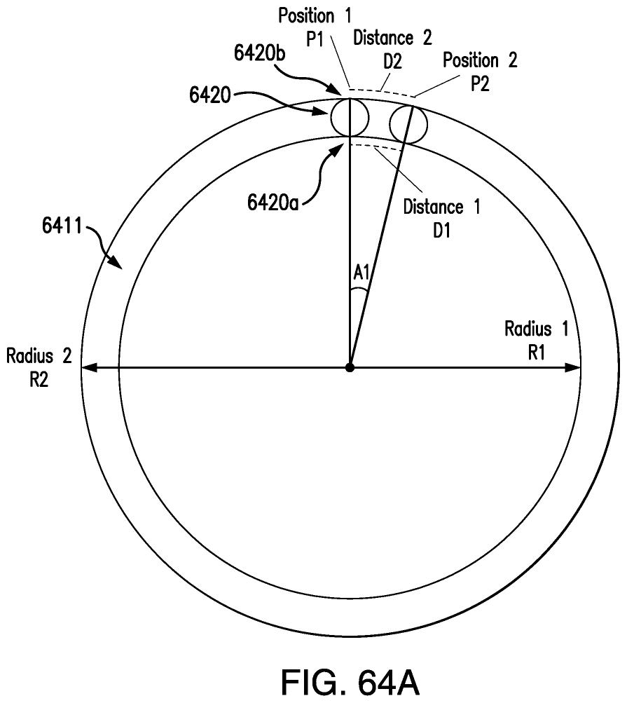

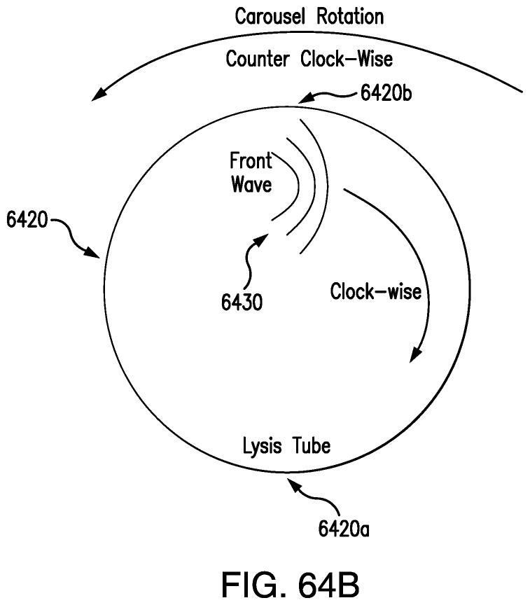

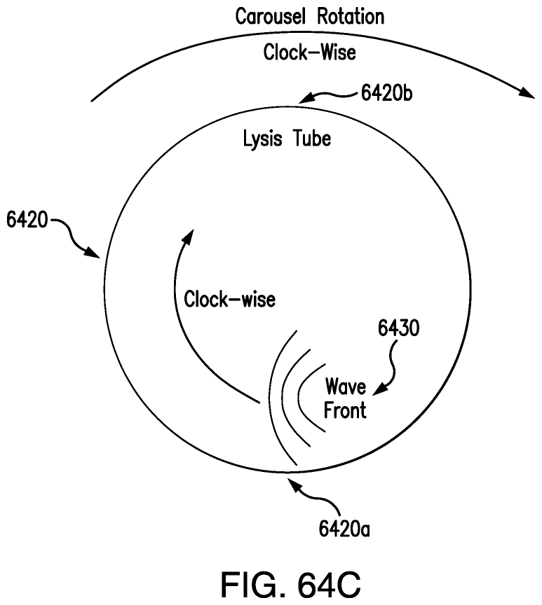

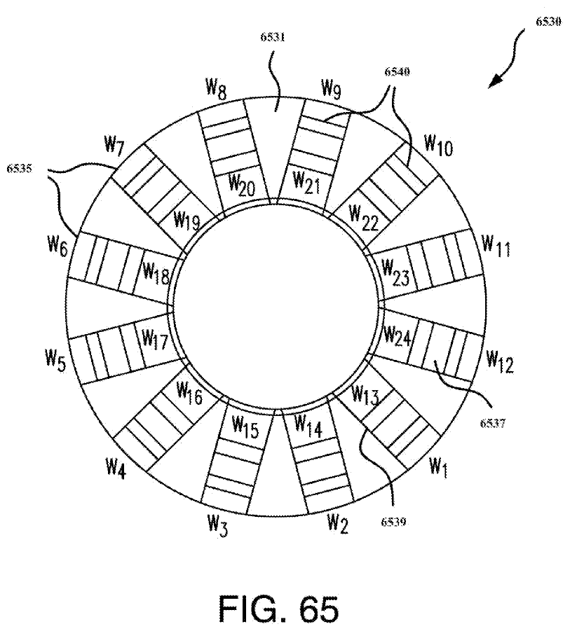

FIGS. 64-65 depict an example sample preparation system according to one aspect of the present disclosure incorporating a sample transport and a wash and elution system. In addition, for purpose of illustration and not limitation, FIGS. 5-7 depict an exemplary sample preparation system with an alternative sample preparation process as contemplated by the present disclosure allowing for distinct sample transfer and rotation timing. Sample preparation is generally employed to isolate and/or purify nucleic acid present in the sample and can include a lysis process and a wash process. In certain embodiments, the lysis process includes the steps of sample lysis and nucleic acid capture. In certain embodiments, the wash process includes nucleic acid wash (e.g., one or more wash steps) and nucleic acid elution (e.g., one or more elution steps).

### 6.1.1 Sample Lysis and Nucleic Acid Capture Systems

As described above, a sample preparation process for use herein can include a lysis process. As described above, sample lysis disrupts the membranes or walls of pathogens, infectious agents, and/or cells present within a sample to release the nucleic acids present within the pathogens, infectious agents, and/or cells present in the sample. Generally, systems are employed to automatically contact a lysis solution with a donor sample to be analyzed. In certain embodiments, the present disclosure is directed to systems configured to contact a donor sample with a sample lysis buffer.

In certain embodiments, as shown in FIG. 64, the sample transport 6410 is a sample preparation carouse, e.g., rotatable carousel 6411 having positions to hold lysis tubes 6420. The lysis tube 6420 holds the sample and the lysis buffer during the lysis process. Although FIG. 64 shows the sample transport 6410 as a single carousel, the lysis process can be separated into two or more process paths. For example, in FIGS. 5-7, the particular systems configured to initiate contact of a sample with a sample lysis buffer are referred herein as “Pre-Lysis systems,” while the systems configured to incubate the sample in the presence of a sample lysis buffer are referred herein as “Lysis systems.” Thus, Pre-Lysis systems generally combine the sample from an individual donor with sample lysis buffer and microparticle-containing reagents, which then undergo lysis and nucleic acid capture in the Sample Lysis system.

FIG. 64 comprises 22 positions (clockwise around the central carousel) capable of hold lysis tubes 6420. In the example system of FIG. 64, the carousel operates on a lock step principle where the carousel moves one position in a clockwise direction after each lockstep. The lockstep, or the time between movements of the carousel, is 24 seconds in this embodiment. Not all positions on the carousel 6411 are used in the sample lysing process in that some positions are used to load the lysis tube onto the carousel, dispense lysis buffer or remove the lysis tube. The below table, Table 3, depicts exemplary times and operations regarding the lysis process. As shown in this exemplary embodiment using a lockstep of 24 seconds, the sample processing time for the lysis process is 384 seconds, or 6.4 minutes.

TABLE 3

| | | Sample Process |
|---|---|---|
| Pos. | Function | Time (Seconds) |
| L1 | Load Lysis Tube in Carousel | |
| | Load Transfer Tip in Carousel | |
| L2 | Dispense 60° C. Lysis Buffer | |
| | (100-1500 μl +/− 5% ) | |
| L4 | Aspirate uParticlesSip | |
| | (30 μl +/− 5%) | |
| | Aspirate Internal ControlSip | |
| | (50 μl +/− 5%) | |
| | Aspirate Proteinase K (only HxV) | |
| | Dispense into LysisTube | |
| | Wash Probe | |
| L5 | Aspirate & Dispense Sample | 24 |
| | Wash Probe | |
| | (100-1000 μl +/− 5%) | |
| L6-18 | Incubation (312 sec) (60° C.) | 312 |
| | Mixing | |
| | Indexing | |
| L19 | Pre-collect μParticles | 24 |
| L20 | Pickup Transfer Tip | 24 |
| | Capture μParticles | |
| | Transfer μParticles | |
| L21 | Aspirate Lysis contents | |
| L22 | Transfer Lysis Tube to Waste | |

In the exemplary embodiment illustrated in FIG. 64 and disclosed in Table 3, position L1 corresponds to the loading of a lysis tube 120 into the process queue. In certain embodiments, loading is accomplished by known “Pick & Place” strategies from a loadable stack. L2 corresponds to a lysis buffer dispensing position. L4 corresponds to dispensing microparticles in the lysis tube. Reagents dispensed at this position can be dispensed via known “Sip & Spit” strategies from reagent containers. Reagents dispensed at this position include, but are not limited to: internal controls, e.g., dispensed at a volume of 50p (+/−5%) although other volumes are contemplated within the scope of the instant disclosure, microparticles, e.g., dispensed at a volume of 30p (+/−5%) or 60 μl (+/−5%) although other volumes are contemplated within the scope of the instant disclosure, Proteinase K, e.g., dispensed at a volume of about 20 μl to about 100 μl, at a volume of about 40 μl (+/−5%) or at a volume of about 20 μl (+/−5%) although other volumes are contemplated within the scope of the instant disclosure. In certain aspects, the time to perform the procedures corresponding to exemplary positions L1, L2, and L4 is not considered in the calculation of TTR. For example, but not by way of limitation, the time to perform the procedures corresponding to exemplary positions L1, L2, and L4 are not considered in the calculation of the duration of the lysis process.

Exemplary position L5 disclosed in Table 3 corresponds to a sample dispensing position. Sample dispensed at this position can be dispensed via known “Sip & Spit” strategies from sample containers. As shown in FIG. 62, sample from an individual donor or patient is aspirated from a sample tube 6204 within a rack of sample tubes 6202. In certain embodiments, the sample is aspirated from an open sample tube. In certain embodiments, the sample is aspirated from a closed sample tube, e.g., via a system capable of piercing the closed sample tube to facilitate aspiration. Samples dispensed at this position can be dispensed at a volume of 100-1000 μl (+/−5%) although other volumes are contemplated within the scope of the instant disclosure.

Exemplary positions L6-L18 disclosed in Table 3 correspond to incubation, mixing, and indexing positions. For example, incubation, mixing, and indexing at positions L6-L18 can incorporate the use of resistive heaters, carousel movement, pop-up mixers, lock step transfers, and/or time priority scheduling. In certain embodiments positions L6-L18 can incorporate incubation in one or more sample lysis buffer. In certain embodiments, the sample can be incubated, mixed, and indexed for about 3 minutes to about 6 minutes, about 4 minutes to about 6 minutes, or about 5 minutes to about 6 minutes. In certain embodiments, the sample can be incubated, mixed, and indexed for about 3 minutes, about 4 minutes, about 5 minutes, or about 6 minutes. In certain embodiments, the sample can be incubated, mixed, and indexed for about 312 seconds, or about 5.2 minutes.

In certain embodiments, the sample lysis buffers used in the methods and systems described herein comprise about 2.5 to about 4.7M GITC, about 2% to about 10% Tween-20, and a pH of about 5.5 to about 8.0. In certain embodiments, e.g., embodiments relating to plasma or serum samples, the sample lysis buffer comprises about 4.7M GITC, about 10% Tween-20, and a pH of about 7.8. In certain embodiments, e.g., embodiments relating to whole blood samples, the sample lysis buffer comprises about 3.5M GITC, about 2.5% Tween-20, and a pH of about 6.0. In certain embodiments, L2-L16 will employ a heater, e.g., a resistive heater, to heat the lysis sample to about 50° C. to about 60° C. In certain embodiments, mixing of the samples is achieved via offline orbital mixing at about 1500 rpm.

Exemplary position L19 disclosed in Table 3 corresponds to a microparticle capture wherein a magnet captures the microparticles at the bottom of a lysis tube 120 to collect the microparticles. This serves to collect the microparticles together before transferring to the wash and elute system 6530. L20 corresponds to a microparticle capture and a transfer position. In this position, the microparticles are released from the bottom of the lysis tube and are then recaptured using other methods. In certain embodiments, microparticles are captured within the transfer tip via magnetic capture against structural elements of the transfer tip. In certain embodiments, such structural elements can include but are not limited to fins, e.g., for magnetic capture of microparticles between such fins. In certain embodiments, microparticles are transferred from the transfer tip to a subsequent process container (e.g., a well within the wash and elute subsystem) by positioning the transfer tip containing with the trapped microparticles into or over the container, retracting the capture magnet and shaking or vibrating the tip.

In certain embodiments, exemplary position L21 (e.g., disclosed in Table 3) corresponds to an aspiration of the lysis contents. In certain embodiments, exemplary position L22 corresponds to transfer of the lysis tube to waste. In certain aspects, the time to perform the procedures corresponding to exemplary positions L21 and L22 is not considered in the calculation of TTR. In certain embodiments, the time to perform the procedures corresponding to exemplary positions L21 and L22 is not considered in the calculation of the duration of the lysis process.

In certain embodiments, the positions of the carousel can be shifted as shown in Table 3.1. For example, and not by way of limitation, in certain embodiments exemplary position L3 corresponds to dispensing microparticles in the lysis tube; exemplary position L4 corresponds to a sample dispensing position; exemplary positions L5-L17 correspond to incubation, mixing, and indexing positions; exemplary position L18 corresponds to a microparticle capture position; exemplary position L19 corresponds to a microparticle capture and a transfer position; exemplary position L20 corresponds to an aspiration of the lysis contents; and exemplary position L21 corresponds to transfer of the lysis tube to waste. In certain aspects, the time to perform the procedures corresponding to exemplary positions L1, L2, L3, L20 and L21 is not considered in the calculation of TTR. In certain embodiments, the time to perform the procedures corresponding to exemplary positions L1, L2, L3, L20 and L21 of a carousel is not considered in the calculation of the duration of the lysis process.

In the above exemplary system, where a lockstep of 24 seconds is used, the sample is processed on the sample transport for 16 locksteps (L5-L20 as shown in Table 3 or L4-L19 as shown in Table 3.1), which results in a total processing time on the sample transport of 384 seconds, or 6.4 minutes.

In certain embodiments, the processing time of the sample on the sample transport starts with aspiration of the sample from the sample vessel (e.g., exemplary positions L4 or L5) and ends with the transfer of the microparticles to the wash vessel (e.g., exemplary positions L19 or L20). In certain embodiments, the total sample processing time on the sample transport can be from about 4 minutes to about 8 minutes, from about 5 minutes to about 7 minutes, or from about 6 minutes to about 7 minutes. In certain embodiments, the total sample processing time on the sample transport can be about 4 minutes, about 5 minutes, about 6 minutes, about 7 minutes, or about 8 minutes. In certain embodiments, the total sample processing time on the sample transport can be about 384 seconds, or about 6.4 minutes.

In certain embodiments of the TTR calculation, the processing time of the sample on the sample transport does not include loading of the lysis tube into the process queue (e.g., exemplary position L1), dispensing a lysis buffer (e.g., exemplary position L2), dispensing microparticles into the lysis tube (e.g., exemplary positions L3 or L4 of Table 3.1 and 3, respectively), aspiration of the lysis contents (e.g., exemplary positions L20 or L21 of Table 3.1 and 3, respectively), and transferring the lysis tube to waste (e.g., exemplary positions L21 or L22 of Table 3.1 and 3, respectively).

For purpose of example and not limitation, the below table, Table 3.1, depicts another exemplary lysis process, including exemplary times and operations with regarding the lysis process. As shown in this exemplary embodiment using a lockstep of 24 seconds, the sample processing time for the lysis process is 384 seconds, or 6.4 minutes (e.g., the total time spent at positions L4-L19).

TABLE 3.1

| | | Sample Process |
|---|---|---|
| Pos. | Function | Time (Seconds) |
| L1 | Load Lysis Tube in Carousel | |
| | Load Transfer Tip in Carousel | |
| L2 | Dispense 60° C. Lysis Buffer | |
| | (100-1500 μl +/− 5%) | |
| L3 | Aspirate uParticlesSip | |
| | (30 μl +/− 5%) | |
| | Aspirate Internal ControlSip | |
| | (50 μl +/− 5%) | |
| | Aspirate Proteinase K (only HxV) | |
| | Dispense into LysisTube | |
| | Wash Probe | |
| L4 | Aspirate & Dispense Sample | 24 |
| | Wash Probe | |
| | (100-1000 μl +/− 5%) | |
| L5-17 | Incubation (312 sec) (60° C.) | 312 |
| | Mixing | |
| | Indexing | |
| L18 | Pre-collect μParticles | 24 |
| L19 | Pickup Transfer Tip | 24 |
| | Capture μParticles | |
| | Transfer μParticles | |
| L20 | Aspirate Lysis contents | |
| L21 | Transfer Lysis Tube to Waste | |

In an alternative embodiment, the sample transport can be performed in separate carousels. For example, but not limitation, and as embodied in FIG. 5, a first carousel can include six positions (clockwise around the central carousel), which can be identified as Pre-Lysis 1 (“PL1”) through Pre-Lysis 6 (“PL6”). Embodiments of the present disclosure can comprise greater or fewer than six of such Pre-Lysis positions, depending on alternative system configurations.

In the exemplary embodiment illustrated in FIG. 5, position PL1 corresponds to the loading of a lysis tube into the process queue. In certain embodiments, loading is accomplished by known “Pick & Place” strategies from a loadable stack. PL2 corresponds to a reagent dispensing position. Reagents dispensed at this position can be dispensed via known “Sip & Spit” strategies from reagent containers. Reagents dispensed at this position include, but are not limited to: internal controls, e.g., dispensed at a volume of 50l (+/−5%) although other volumes are contemplated within the scope of the instant disclosure, microparticles, e.g., dispensed at a volume of 20-40 μl (+/−5%) although other volumes are contemplated within the scope of the instant disclosure, Proteinase K, e.g., dispensed at a volume of 50 μl (+/−5%) although other volumes are contemplated within the scope of the instant disclosure.

Exemplary position PL3 corresponds to a sample dispensing position. Sample dispensed at this position can be dispensed via known “Sip & Spit” strategies from sample containers. As shown in FIG. 5, sample from an individual donor or patient is aspirated from a sample tube 8 within a rack of sample tubes 9. In certain embodiments, the sample is aspirated from an open sample tube. In certain embodiments, the sample is aspirated from a closed sample tube, e.g., via a system capable of piercing the closed sample tube to facilitate aspiration. Samples dispensed at this position can be dispensed at a volume of 100-1000 μl (+/−5%) although other volumes are contemplated within the scope of the instant disclosure.

Exemplary position PL4 corresponds to a sample lysis buffer dispensing/mixing position. Reagents dispensed at this position and be dispensed via direct plumb from a bulk reservoir or via known “Sip & Spit” strategies from reagent containers. Sample lysis buffer dispensed at this position can be dispensed at a volume of 100-1000 μl (+/−5%) although other volumes are contemplated within the scope of the instant disclosure.

Exemplary position PL5 corresponds to a lysis tube sealing position. Sealing of lysis tubes at this position can be accomplished via known methods in the art, e.g., heat stake tape, press on caps, and PSA tape.

Exemplary position PL6 corresponds to a lysis tube transfer position. Transfer of lysis tubes at this position can be accomplished via known methods in the art, e.g., sideways shuffle strategies and/or “Pick & Place” strategies. In certain embodiments, tubes transferred from the Pre-Lysis to Lysis carousels are sealed to minimize carryover and/or splashing.

The below table, Table 4, depicts exemplary operations regarding the Pre-Lysis process.

TABLE 4

| Pos. | Function |
|---|---|
| PL1 | Load Lysis Tube in Carousel |
| PL2 | Dispense Internal Control |
| | (50 μl +/− 5%) |
| | Wash Probe |
| | Dispense μParticles |
| | (20 μl-40 μl +/− 5%) |
| | Dispense Proteinase K (only HxV) |
| | (50 μl +/− 5%) |
| | Wash Probe |
| PL3 | Dispense Sample |
| | (100-1500 μl +/− 5%) |
| | Wash Probe |
| PL4 | Dispense 60° C. Lysis Buffer |
| | (100-1500 μl +/− 5%) |
| PL5 | Seal Lysis tube |
| PL6 | Transfer Lysis tube to Sample Lysis Carousel |

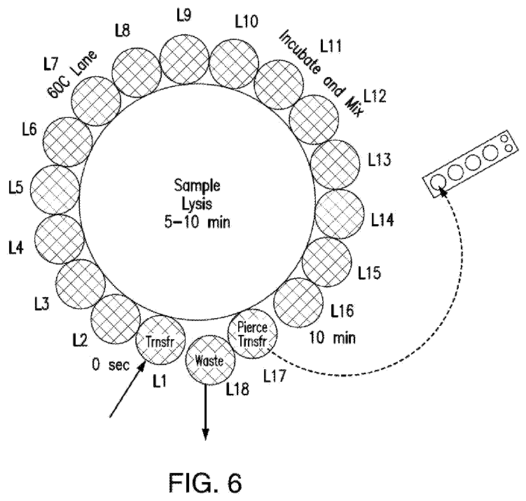

FIG. 6 is a diagram illustrating an exemplary Sample Lysis system according to the disclosed subject matter. Generally, the Sample Lysis system provides the sample the proper time, temperature and mixing for lysis and capture.

FIG. 6 comprises 18 positions (clockwise around the central carousel), which can be identified as Lysis 1 (“L1”) through Lysis 18 (“L18”). Embodiments of the present disclosure can comprise greater or fewer than 18 positions, depending on alternative system configurations and desired capacity and throughput.

In the exemplary embodiment illustrated in FIG. 6, position L1 corresponds to a transfer position where the lysis tube is transferred to the carousel from, e.g., a Pre-Lysis transfer position. Transfer of lysis tubes to this position can be accomplished via known methods in the art, e.g., sideways shuffle strategies and/or “Pick & Place” strategies. Exemplary positions L2-L16 correspond to incubation, mixing, and indexing positions. For example, incubation, mixing, and indexing at positions L2-L16 can incorporate the use of resistive heaters, carousel movement, pop-up mixers, lock step transfers, and/or time priority scheduling. In certain embodiments, L2-L16 will employ lock step transfers, e.g., about every 24 seconds (or longer in some instances), to achieve a total incubation duration of about 300 to about 600 seconds although other durations are contemplated within the scope of the instant disclosure. In certain embodiments positions L2-L16 can incorporate incubation in one or more sample lysis buffer.

In certain embodiments, the sample lysis buffers used in the methods and systems described herein comprise about 2.5 to about 4.7M GITC, about 2% to about 10% Tween-20, and a pH of about 5.5 to about 8.0. In certain embodiments, e.g., embodiments relating to plasma or serum samples, the sample lysis buffer comprises about 4.7M GITC, about 10% Tween-20, and a pH of about 7.8. In certain embodiments, e.g., embodiments relating to whole blood samples, the sample lysis buffer comprises about 3.5M GITC, about 2.5% Tween-20, and a pH of about 6.0. In certain embodiments, L2-L16 will employ a heater, e.g., a resistive convection heater, to heat the lysis sample to about 50° C. to about 60° C. In certain embodiments, mixing of the samples is achieved via offline orbital mixing at about 1500 rpm.

Exemplary position L17 corresponds to a microparticle capture and transfer position. Capture and transfer of microparticles at position L17 can involve the use of a pickup transfer tip and piercing of the lysis tube seal. In certain embodiments, the transfer tip pickup is accomplished by known “Pick & Place” strategies from, e.g., loadable racks. In certain embodiments, the lysis tube is pierced by pushing the transfer tip through the lysis tube seal. In certain embodiments, hole clearance can be increased by twisting the transfer tip during or after the lysis tube seal is pierced. In certain embodiments, microparticles are captured within the transfer tip via magnetic capture against structural elements of the transfer tip. In certain embodiments, such structural elements can include but are not limited to fins, e.g., for magnetic capture of microparticles between such fins. In certain embodiments, microparticles are transferred from the transfer tip to a subsequent process container (e.g., a well within the Wash and Elute system) by positioning the transfer tip containing with the trapped microparticles into or over the container, retracting the capture magnet and shaking or vibrating the tip.

FIG. 6 comprises 18 positions (clockwise around the carousel) and the carousel operates on a lock step principle where the carousel moves one position in a clockwise direction after each lockstep. The lockstep, or the time between movements of the carousel, is 24 seconds in this embodiment. The below table, Table 5, depicts exemplary times and operations regarding the lysis process. As shown in this exemplary embodiment using a lockstep of 24 seconds, the sample processing time for the lysis process is 300-600 seconds, or 5-10 minutes.

TABLE 5

| | | Sample Process |
|---|---|---|
| Pos. | Function | Time (Seconds) |
| L1 | Transfer Lysis Tube to Lysis Carousel | |
| L2-L16 | Incubation (312 sec) (60° C.) | 300-600 |
| | Mixing | |
| | Indexing | |
| L17 | Pickup Transfer Tip | |
| | Pierce Lysis Tube | |
| | Capture μParticles | |
| | Transfer μParticles | |
| L18 | Transfer Lysis Tube to Waste | |

For purpose of example and not limitation, the below table, Table 5.1, depicts another exemplary lysis process embodiment using a lockstep of 24 seconds, the sample processing time for the lysis process is 300-600 seconds, or 5-10 minutes.

TABLE 5.1

| | | Sample Process |
|---|---|---|
| Pos. | Function | Time (Seconds) |
| L1 | Transfer Lysis Tube to Lysis Carousel | |
| L2-L16 | Incubation (312 sec) (60° C.) | 300-600 |
| | Mixing | |
| | Indexing | |
| L17 | Pickup Transfer Tip | |
| | Pierce Lysis Tube | |
| | Capture μParticles | |
| | Transfer μParticles | |
| L20 | Aspirate Lysis contents to waste | |
| L21 | Transfer Lysis Tube to Waste | |

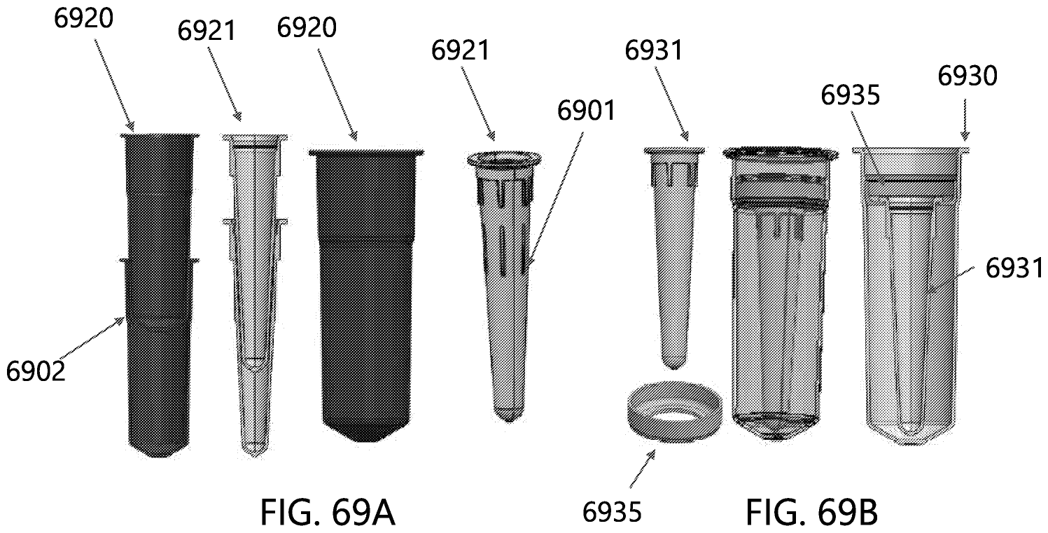

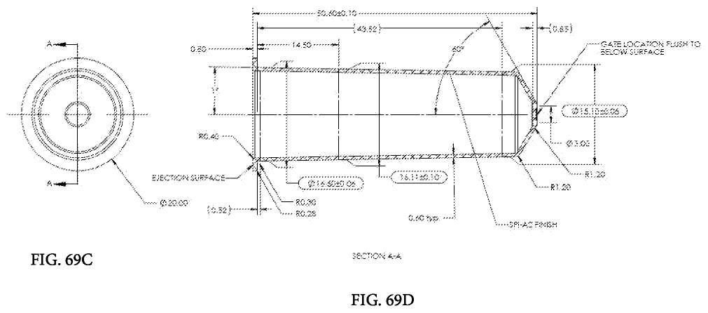

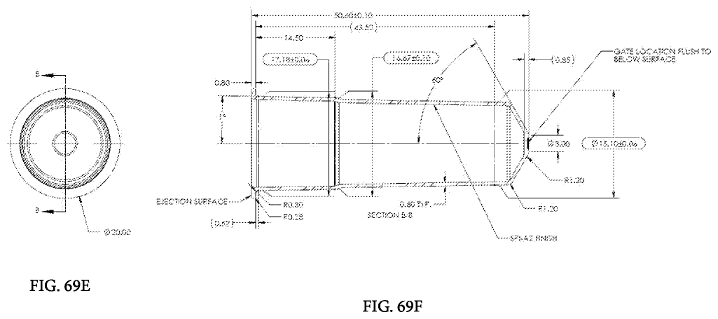

For purpose of example and not limitation, FIGS. 69A and 69B depict various embodiments of lysis tubes and transfer tips for use in the systems and methods described herein. For purpose of example and as embodied herein, lysis tube 6920 can include a conical bottom. The conical bottom can include any suitable draft angle. For purpose of example and as embodied herein, lysis tube 6920 can have a conical bottom with a draft angle of approximately 1 degree. For purpose of example and as described further herein, the configuration of the lysis tube bottom can be selected to achieve desired mixing performance, such as for example, when lysis mixing is performed by successive oscillation of the sample transport, e.g., lysis carousel. As further embodied herein, lysis tube 6920 can include an anti-nesting feature 6902. For purpose of example and as embodied herein, the anti-nesting feature 6902 can include an area of reduced tube diameter, and when multiple lysis tubes are stacked together, the bottom of a first lysis tube can engage with the anti-nesting feature 6902 of a second lysis tube to prevent the first lysis tube from nesting completely within the second lysis tube. Having a first lysis tube completely nested within a second lysis tube can be undesirable, as the first lysis tube can become wedged or stuck within the second lysis tube, making it more challenging to retrieve a single lysis tube from a stack of lysis tubes. For purpose of example, lysis tubes 6920 can have anti-nest features illustrated as 6902 and be packed into individual wells. For purpose of illustration not limitation, the tubes can be stacked together without touching the bottom portion of each tube to avoid contamination and for easier transportation. Additionally, or alternatively, the sidewalls of tubes can include matching grooves to avoid movements among stacking tubes. As further embodied herein, transfer tip 6920 can include an anti-nesting feature 6901. For purpose of example and as embodied herein, anti-nesting features 6901 can include one or more protrusions arranged around an outer sidewall of the transfer tip 6920. As embodied herein, when multiple transfer tips are stacked together, the one or more protrusions of a first transfer tip 6920 can engage with the inner sidewall of a second transfer tip to prevent the first transfer tip 6920 from nesting completely within the second transfer tip. For purpose of example, and as embodied herein, the materials for the lysis tubes and transfer tips can be medical-grade copolymer PP.

Additionally or alternatively, and as further embodied herein, lysis tubes and transfer tips can be configured to be stacked together in a single stack. For example, and as depicted in FIG. 69B, transfer tip 6931 can be concentrically arranged within lysis tube 6930 for stacking. As embodied herein, a centering ring 6935 can be used to center transfer tip 6931 within lysis tube 6930 for stacking. For purpose of example and as embodied herein, the centering ring 6935 can be pressed into the lysis tube 6930, and transfer tip 6931 can be held in position within the lysis tube 6930 by the centering ring 6935. For example and as embodied herein, centering ring 6935 can engage with a flange on the transfer tip 6935. As embodied herein, concentrically stacking lysis tubes and transfer tips together can improve packaging density. For example and not limitation, a single stack of lysis tubes and transfer tips can be used rather than separate stacks of lysis tubes and transfer tips, respectively. Additionally or alternatively, centering ring 6935 can reduce splashing of liquid contents out of the lysis tube 6930, for example during sample preparation.

For purpose of example and illustration and not limitation, additional lysis tube embodiments are depicted in FIGS. 69C-69F, along with exemplary dimensions for the lysis tubes shown. As described further herein, lysis tube dimensions can be selected according to the desired performance of the system. For example, lysis tube dimensions can be selected to facilitate mixing of the contents of the lysis tube and to reduce the risk that the contents of the lysis tube will splash out of the tube during mixing operations. Additionally or alternatively, and as embodied herein, lysis tube dimensions can be selected to achieve desired stacking efficiency.

### 6.1.2 Mixing Systems

As described above, sample preparation processes for use herein can include, for example, a pre-treatment process (e.g., a pre-treatment lysis process), lysis process, an onboard pooling process, and combinations thereof. For example and as embodied herein, sample preparation processes can be carried out in a sample preparation area, and the sample preparation area can include a sample transport. Sample processes for use herein, can include mixing. For example and not limitation, a lysis process can include mixing to mix a sample and reagents in a lysis tube. Additionally or alternatively, an onboard pooling process can include mixing two or more samples in a vessel, such as for example to distribute the two or more samples more evenly within the pooled sample.

As described above, mixing of the contents of vessels or lysis tubes 6420 in a sample preparation process can occur on the sample transport. For example and not limitation, mixing can occur at one or more positions on the lysis carousel 6411. Mixing of the contents of lysis tubes 6420 can be desired, for example, to mix a sample and lysis buffer within the lysis tube. Mixing can be performed, for example, using mechanical agitation, such as with a mechanical tip, which can be inserted within the lysis tube 6420. Additionally or alternatively, mixing can be performed using a mechanism, such as a vortexer, which can interface with the lysis tube 6420 to, for example, vibrate and/or rotate the lysis tube 6420 to mix its contents. Additionally or alternatively, mixing can be performed using magnets, which can interact with, for example, magnetic particles within the lysis tube 6420 to agitate the contents of the lysis tube 6420. For example, permanent magnets can be moved relative to the lysis tube and can agitate magnetic particles within the lysis tube 6420. Additionally or alternatively, electro-magnets can be positioned adjacent to the lysis tube 6420 and used to agitate magnetic particles within the lysis tube 6420.

Additionally or alternatively, and as embodied herein, mixing can be performed using successive oscillation of the sample transport. For example and as embodied herein, the sample transport can be configured to transport one or more samples in a vessel along a transport path from a sample dispense position to a sample capture and transfer position, as described further herein. For example and not limitation, the sample transport can include a serpentine path, conveyor, such as a chain conveyor, extrusion, robotic handler, belt, or vehicle system. As embodied herein, the sample transport includes a sample preparation carousel, such as a lysis carousel. Successive oscillation of the sample transport can be performed with the vessel to be mixed moving in an arc with each oscillation of the sample transport. Movement in an arc can include at least two axis of movement. For example and as embodied herein, movement in an arc can include successive clockwise and counterclockwise rotation of a carousel. For example and not limitation, mixing of the contents of the lysis tubes 6420 can be performed using rotational movement of rotatable carousel 6411. For example, carousel 6411 can be rotated clockwise and counterclockwise in quick succession and the rotation of the carousel 6411 can cause the contents of lysis tubes 6420 to mix. Additionally or alternatively, movement in an arc can include successive movement in one direction followed by movement in another direction, such as for example, successive movement of a belt in a first direction followed by movement in a second direction along a portion of the belt defining an arc, e.g., along a portion comprising movement in at least two axis.

Without wishing to be bound by theory, exemplary physics principles of mixing which can be performed using successive oscillations, e.g., successive rotational movement of rotatable carousel 6411, are explained with reference to FIG. 64A. For purpose of illustration and explanation and not limitation, rotatable carousel 6411 is depicted with a single lysis tube 6420, which can be moved from position P1 to position P2 by rotation of rotatable carousel 6411. As shown, a first radius, R1, can be measured between a center of the rotatable carousel 6411 and a portion of the wall 6420a of lysis tube 6420 closest to the center of the rotatable carousel 6411, and a second radius, R2, can be measured between a center of the rotatable carousel 6411 and a portion of the wall 6420b of lysis tube 6420 farthest from the center of the rotatable carousel 6411. As embodied herein R2 can be greater than R2. The rotatable carousel 6411 can rotate lysis tube 6420 from position P1 to position P2. As shown, inner wall portion 6420a travels a distance D1 during rotation of the lysis tube 6420 from position P1 to position P2 and outer wall portion 6420b travels a distance D2 during rotation of the lysis tube 6420 from position P1 to position P2. As embodied herein, during rotation of carousel 6411 and movement of lysis tube 6420 from position P1 to position P2, the inner wall portion 6420a can travel distance D1 in the same amount of time it takes outer wall portion 6420b to travel D2. Accordingly, and as embodied herein, acceleration of outer wall portion 6420b during rotation of the carousel 6411 and movement of lysis tube 6420 from position P1 to position P2 is greater than the acceleration of the inner wall portion 6420a during rotation of the carousel 6411 and movement of lysis tube 6420 from position P1 to position P2. In other words, as embodied herein, during rotation of carousel 6411, the distance traveled on the outside of the carousel is greater than the distance traveled on the inside of the carousel, and since both distances are traveled simultaneously, the acceleration is greater on the outside of the carousel than on the inside of the carousel.

For purpose of example and explanation and not limitation, exemplary calculations are included herein to further illustrate and explain the operation of the exemplary embodiment. For example, the distance traveled on the inside of the carousel during rotation of the carousel from position P1 to position P2 can be represented as an arc having an arc length D1 and the distance traveled on the outside of the carousel can be represented as an arc having an arc length D2. For purpose of explanation and illustration, and as embodied herein, carousel 6411 can have an inner radius R1 of approximately 117 mm and an outer radius R2 of approximately 133 mm, and position P1 and position P2 can be approximately 1.33 degrees of rotation apart. For purpose of explanation and illustration the arc length D1 can be calculated as 2*R1*π*1.33/360. D2 can be calculated as 2*R2*π*1.33/360. For purpose of explanation and illustration, and as embodied herein, D1 can be approximately 2.72 mm and D2 can be approximately 3.08 mm.

As described above, during rotational movement of the carousel 6411 from position P1 to position P2, inner wall portion 6420a travels distance D1 in the same amount of time outer wall portion 6420b travels distance D2. For purpose of explanation and illustration and not limitation, the acceleration of the inner wall portion 6420a during rotation from position P1 to position P2 over time “t” can be calculated as acceleration 1=2*D1/t² and the acceleration of outer wall portion 6420b can be calculated as acceleration 2=2*D2/t². For purpose of example, and as embodied herein, for a D1 of 2.72 mm and a D2 of 3.08 mm, acceleration 1 of the inner wall portion 6420a can be approximately 88% of acceleration 2 of the outer wall portion 6420b.

As described above and as embodied herein, acceleration of outer wall portion 6420b during rotation of the carousel 6411 and movement of lysis tube 6420 from position P1 to position P2 can be greater than the acceleration of the inner wall portion 6420a during rotation of the carousel 6411 and movement of lysis tube 6420 from position P1 to position P2. As embodied herein, greater acceleration of the outer wall portion 6420b can be related to the diameter of the lysis tube 6420. For example, and with reference to FIG. 64A, the difference between R2 and R1 can be related to lysis tube diameter, with lysis tubes having larger diameters corresponding to a larger difference between R2 and R1 and a larger difference in acceleration between the outer wall portion 6420b and the inner wall portion 6420a. For example and not limitation, lysis tubes having a lower draft angle at the bottom of the tube can have a larger diameter at the bottom of the lysis tube, which can provide a corresponding larger difference in acceleration between the outer wall portion 6420b and the inner wall portion 6420a. For purpose of example and not limitation, a lysis tube with a 1° draft angle will have a larger tube diameter towards the bottom of the tube than a lysis tube with a 2.5° draft angle. Further, and as described further herein, a larger lysis tube diameter can result in a lower fluid height within the lysis tube, which can, for example, reduce the risk of liquid splashing out of the lysis tube during mixing.

FIG. 64B depicts a schematic view of a lysis tube 6420 during counter-clockwise rotation of carousel 6411 from a first position to a second position. As described above, during rotation of carousel 6411 from a first position to a second position, outer wall portion 6420b can accelerate faster than inner wall portion 6420a. This difference of accelerations between inner wall portion 6420a and outer wall portion 6420b can cause liquid contained in lysis tube 6420, such as, for example, a sample and lysis buffer, to rotate and create a wave in the tube during movement of the carousel 6411 from a first position to a second position. For purpose of example and not limitation, FIG. 64B depicts a liquid wave front 6430 forming in lysis tube 6420 along outer wall portion 6420b during counter-clockwise rotation of carousel 6411. As shown, the wave front 6430 can travel along the outer wall of the lysis tube 6420 in a direction opposite to the rotational movement of the carousel 6411. For example, as depicted in FIG. 64B, a counter-clockwise rotation of the carousel 6411 can cause wave front 6430 to form and travel in a clockwise direction along the outer wall portion 6420b of the lysis tube 6420.

As further embodied herein, for purpose of example and not limitation, reversing the direction of rotation of carousel 6411 can further contribute to mixing of the contents of the lysis tubes 6420. For example, and with reference to FIG. 64C, lysis tube 6420 is shown during clockwise rotation of carousel 6411 from position P2 to position P3. As shown for purpose of illustration and discussion, after counter-clockwise rotation of carousel 6411 and lysis tube 6420 from position P1 to position P2, wave front 6430 can travel within the lysis tube 6420 along the outer wall from the outer wall portion 6420b to inner wall portion 6420a. As embodied herein, reversing the rotational direction of the carousel 6411 (e.g., from counter-clockwise rotation to clockwise rotation) when the wave front has traveled from the outer wall portion 6420b to inner wall portion 6420a can impart further rotational forces on the contents of the lysis tube. For example, and as embodied herein, reversing the direction of the carousel with the wave front 6430 along the inner wall portion 6420a can cause the wave front to continue its clock-wise rotation. For purpose of example and as embodied herein, lysis tube 6420 can include liquid, such as for example a whole blood sample and lysis fluid, and magnetic particles as described herein. As embodied herein, rotating the carousel successively in one direction and then the opposite direction (e.g., counter-clockwise followed by clockwise rotation, or vice versa) can form a vortex of magnetic particles within the liquid of the lysis tube 6420. The rotation speed, rotation distance, reversal, and delay timing can be selected based on the desired performance of the system and dimensions of the system, such as, for example, the dimensions of the lysis tube 6420 and dimensions of the carousel 6411. For purpose of example, and as embodied herein, the rotation speed, rotation distance, reversal, and delay timing can be selected to form a vortex within liquid contents of the lysis tube within approximately 1.3 seconds. As embodied herein, the formation of a vortex within liquid contents of the lysis tube can provide an indication of mixing.

Mixing the contents of vessels, e.g., lysis tubes 6420 in a sample preparation process using successive oscillation of the sample transport, such as for example, rotational movement of rotatable carousel 6411, can provide advantages. For example and not limitation, mixing using successive oscillation of the sample transport, such as for example, rotational movement of rotatable carousel 6411 can reduce or eliminate the hardware required to mix the contents of lysis tubes 6420. For example, and as embodied herein, when rotational movement of the carousel is used for mixing, separate mixing hardware, such as magnets and/or a mechanical agitator or vortexer can be omitted. Additionally or alternatively, mixing using rotational movement of rotatable carousel 6411 can reduce the number of consumables used. For example, mixing using rotational movement of rotatable carousel 6411 can reduce or eliminate the need for a disposable mechanical agitator tip. Additionally or alternatively, mixing using rotational movement of rotatable carousel 6411 can reduce or eliminate the need for a splash or aerosol containment covering for the lysis tube, which can further reduce consumable use. Additionally or alternatively, and as further embodied herein, mixing using rotational movement of rotatable carousel 6411 can reduce the time required for mixing and can increase system throughput. For example and not limitation, mixing using rotational movement of rotatable carousel 6411 can reduce or eliminate other mixing steps, such as mechanical agitation, which can require additional processing time.

Additionally or alternatively, mixing using successive oscillation of the sample transport, such as for example, rotational movement of rotatable carousel 6411 can be performed using small successive oscillations, such as for example clockwise and counterclockwise rotation of the rotatable carousel for short distances, such as for example, rotation corresponding to approximately +/−10% of lysis tube diameter. As embodied herein, a robotic pipettor can access vessels on the sample transport during oscillation of the sample transport and vessels on the sample transport. For example and as embodied herein, a robotic pipettor can access lysis tubes on the rotatable carousel during the rotatable mixing operation, which can be advantageous for reducing sample processing times. Additionally or alternatively, and as embodied herein, mixing the contents of the lysis tube using rotational movement of rotatable carousel 6411 can be performed at any of the positions on the carousel.

### 6.1.3 Pooling Systems

In accordance with another aspect of the disclosed subject matter, the disclosed methods and systems can be used with pooled samples to perform a nucleic acid analysis on a pooled sample. For purpose of example and as embodied herein, samples can be pooled using onboard hardware. The term “onboard” as used herein is a broad term, and is to be given its ordinary and customary meaning to a person of skill in the art (and it is not to be limited to a special or customized meaning), and refers without limitation to the inclusion of components and/or the performance of method steps on a single system (e.g., a single piece of laboratory equipment). For example and not limitation, onboard pooling can include pooling samples and performing analysis of the pooled sample on the same piece of laboratory equipment without manual intervention between pooling and analysis.

As embodied herein systems and methods can include onboard pooling of a subset of a plurality of samples being tested or screened to form a pooled sample, and nucleic acid analysis can be performed on the pooled sample, such as to detect the presence of one or more pathogens or infectious agents.

For purpose of example and not limitation, and as embodied herein, the same hardware that manages individual sample preparation can be used for pooling to form pooled samples. For example and as embodied herein, nucleic acid analysis can include a sample preparation process and the sample preparation process can be performed at a sample preparation area. For purpose of example and as embodied herein, onboard pooling can be performed in a sample preparation area of the exemplary HTNAT sample analysis system 6800 as depicted in FIGS. 68A-68D. The sample preparation area can include a sample transport and the sample transport can be configured to continually transport individual vessels along a transport path from a sample dispense position to a sample capture and transfer position, with intermediate positions therebetween. The term “continually transport” as used herein is a broad term, and is to be given its ordinary and customary meaning to a person of skill in the art (and it is not to be limited to a special or customized meaning), and refers without limitation to transporting, transferring, and/or carrying samples from one position to another automatically, and one after the other in a stream-like fashion, wherein at least during steady-state operation, addition of a first vessel to the sample transport at a first position can coincide with removal of another vessel from the sample transport at a second downstream position. The sample transport can include any suitable configuration. For example and not limitation, the sample transport can include a serpentine path, conveyor, such as a chain conveyor, extrusion, robotic handler, belt, or vehicle system. As embodied herein, the sample transport for the exemplary HTNAT sample analysis system 6800 can include a sample preparation carousel 6805, and onboard pooling can be performed on the sample preparation carousel 6805. As embodied herein, the sample preparation carousel can be a lysis carousel. For purpose of example and as embodied herein, the sample preparation carousel 6805 can be configured to continually transport individual vessels along a transport path from a sample dispense position to a sample capture and transfer position, with intermediate positions therebetween. For purpose of example and as embodied herein, the transport path can extend about a perimeter of the sample preparation carousel 6805 between sample dispense position L4 and a sample capture and transfer position L19 with intermediate positions L5-L18 therebetween. As embodied herein, continually transporting individual vessels along the transport path can include rotating the sample preparation carousel 6805. As embodied herein, the sample preparation carousel 6805 can rotate in a lockstep fashion, as described further herein. For purpose of example and not limitation, the duration of each lockstep can be between about 15 and about 30 seconds.

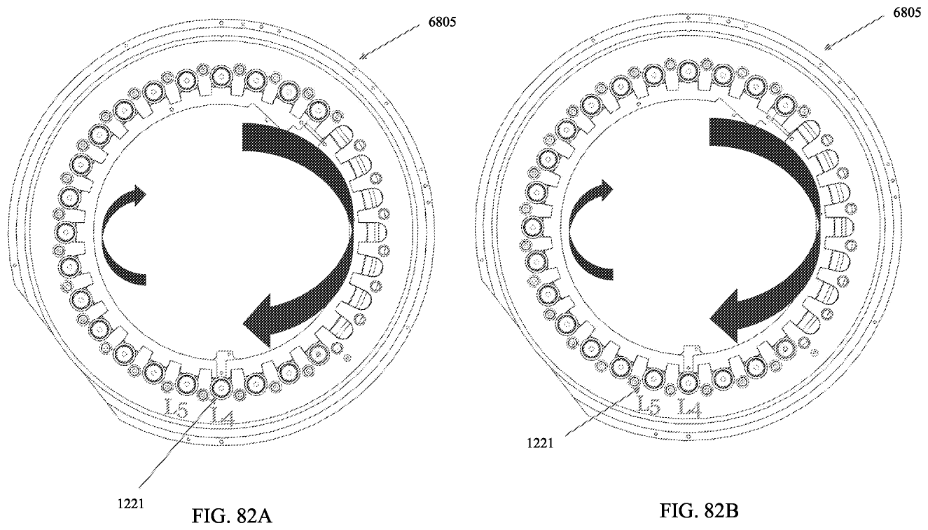

For example and as embodied herein, onboard pooling can include continually transporting individual vessels, such as lysis tubes, on the sample preparation carousel 6805 along a transport path between the sample dispense position L4 to the sample capture and transfer position L18 and pooling a first sample and a second sample in a vessel on the sample preparation carousel 6805 to form a pooled sample. For purpose of example and as embodied herein, pooling a first sample and a second sample can include dispensing the first sample and the second sample into a first vessel 1221 at the sample dispense position L4 prior to the sample preparation carousel 6805 transporting the first vessel 1221 to a first intermediate position L5. As embodied herein the first vessel 1221 can include one or more reagents, such as a lysis buffer, as described herein. For purpose of example and as embodied herein, the sample transport 6805 can move in a lockstep fashion as described above, and with reference to FIGS. 82A and 82B, a sample/reagent pipettor 6811 can transfer a first sample from a first sample tube at a sample loading area to the first vessel 1221 at sample dispense position L4. As further embodied herein, the sample/reagent pipettor 6811 can transfer the second sample from a second sample tube at the sample aspiration position 6892 to the first vessel 1221 at sample dispense position L4 to combine the first and second samples prior to the sample preparation carousel 6805 transporting the first vessel 1221 to a first intermediate position L5. For purpose of example and not limitation, once pooled, the pooled first and second samples in the first vessel 1221 can be transported along the transport path to the sample capture and transfer position L18. For example and as embodied herein, a lysis process can be performed on the pooled first and second samples as described herein.

For purpose of example and not limitation, the first and second samples transferred to the first vessel 1221 at sample dispense position L4 can be pooled samples. For example and not limitation, the first and second samples can each include a pool of 48 samples. As described further herein, the first and second samples can each be a pooled sample, such as a pooled sample that was prepared on a liquid handler or pooler prior to introduction to the sample aspiration position 6892. Additionally or alternatively, the first and second samples can each be an individual donor sample.

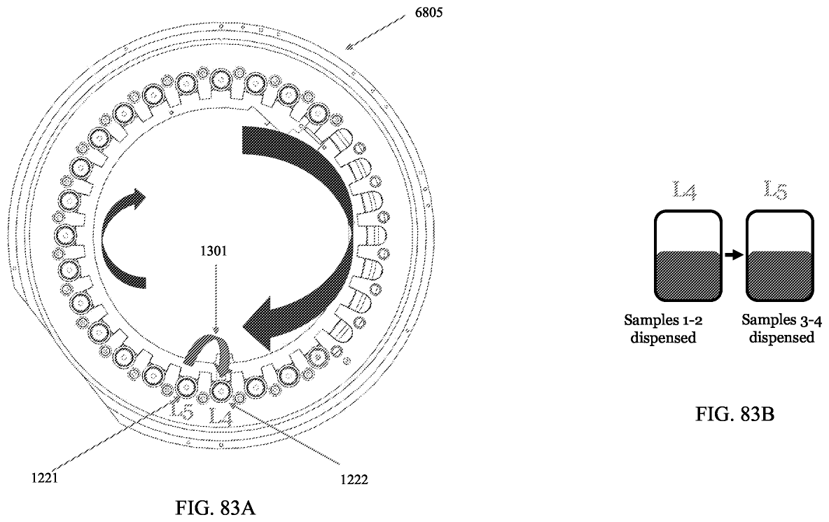

Additionally or alternatively, and as embodied herein, onboard pooling can further include combining and pooling additional samples. For example, onboard pooling can include combining and pooling additional samples in the first vessel 1221. For purpose of example and illustration, reference is made to FIG. 83A, which depicts the sample preparation carousel 6805 with a first vessel 1221 containing a first sample and a second sample therein at an intermediate position L5. For purpose of example and as embodied herein, a third sample and a fourth sample can be dispensed into the first vessel 1221 at intermediate position L5 to add the third and fourth samples to the pooled sample. As embodied herein, pooling the samples can include transferring the third sample from a third sample tube at the sample aspiration position 6892 and a fourth sample from a fourth sample tube at the sample aspiration position 6892 to the first vessel 1221 at the first intermediate position L5.

Additionally or alternatively, and as embodied herein, sample preparation processes in accordance with an aspect of the disclosed subject matter can include a lysis process as described herein, and in certain embodiments, onboard pooling can further include transferring the pooled sample for further sample preparation, such as a lysis process, on the sample preparation carousel 6805. For example and with continued reference to FIG. 83A, the pooled sample can be transferred from an intermediate position on the sample preparation carousel 6805 to a second vessel 1222 at the sample dispense position L4. For purpose of example and as embodied herein, pooled first, second, third, and fourth samples in the first vessel 1221 can be transferred from the first vessel 1221 to a second vessel 1222 at the sample dispense position L4 as represented in FIG. 83A by arrow 1301. For purpose of example, and as described further herein, the first, second, third, and fourth samples can be combined on the sample preparation carousel 6805 in the first vessel 1221, and the pooled sample can then be transferred to a second vessel 1222 having one or more reagents therein at the sample dispense position L4 to initiate the lysis process for the pooled sample.

As embodied herein, individual samples and/or pooled samples can be transferred between vessels on the sample preparation carousel 6805 using the sample/reagent pipettor 6811. For example and as embodied herein, the sample/reagent pipettor 6811 can use disposable tips to aspirate and dispense samples. For purpose of example and illustration, sample/reagent pipettor 6811 can aspirate the pooled first, second, third, and fourth samples from the first vessel 1221 at the first intermediate position L5 and dispense the pooled sample into the second vessel 1222 at the sample dispense position L4 using one disposable pipette tip. Additionally or alternatively, the first, second, third, and fourth samples can be pooled in the second vessel 1222 at the sample dispense position L4. For example, with the first vessel 1221 having the pooled first and second samples at the first intermediate position L5, sample/reagent pipettor 6811 can aspirate the third sample from its sample container at the sample aspiration position 6892 and transfer the third sample to the second vessel 1222 at the sample dispense position L4 using a first disposable pipette tip. The sample/reagent pipettor 6811 can then dispose of the first tip, pick up a second disposable tip and aspirate the fourth sample from its sample container at the sample aspiration position 6892. The sample/reagent pipettor 6811 can then use the same disposable tip to further aspirate the first and second samples from the first vessel 1221 at the first intermediate position L5. The sample/reagent pipettor 6811 can then dispense the first, second, and fourth samples into the second vessel 1222 at the sample dispense position L4 to pool the first, second, third, and fourth samples in the second vessel 1222. As embodied herein, an additional sample preparation processes can then be initiated for the pooled sample.

Additionally or alternatively, and as further embodied herein, additional samples can be pooled. For example and not limitation, additional samples can be transferred from respective sample containers at the sample aspiration position 6892 to the sample preparation carousel 6805 and pooled in the first vessel 1221. For example, additional samples can be transferred from respective sample tubes at the sample aspiration position 6892 to the first vessel 1221 at the sample dispense position L4 prior to the sample transport transporting the first vessel 1221 to the first intermediate position L5. Additionally or alternatively, additional samples can be transferred from respective sample tubes at the sample aspiration position 6892 to the first vessel 1221 at the first intermediate position L5 prior to the sample transport transporting the first vessel 1221 from the first intermediate position L5 to a second intermediate position L6. Additionally or alternatively, additional samples can be transferred from respective sample tubes at the sample aspiration position 6892 to the first vessel 1221 at other intermediate positions along the transport path of the sample preparation carousel 6805. For example and not limitation, one sample can be transferred from a sample tube at the sample aspiration position 6892 to the first vessel 1221 at each intermediate position. Additionally or alternatively, two samples can be transferred from respective sample tubes at the sample aspiration position 6892 to the first vessel 1221 at each intermediate position. Additionally or alternatively, three samples can be transferred from respective sample tubes at the sample aspiration position 6892 to the first vessel 1221 at each intermediate position. Additionally or alternatively, four samples can be transferred from respective sample tubes at the sample aspiration position 6892 to the first vessel 1221 at each intermediate position.

For purpose of example and not limitation, between 2 and 50 samples can be pooled in the first vessel 1221 on the sample preparation carousel 6805 as the first vessel is transported along the transport path. Additionally or alternatively, between 2 and 30 samples can be pooled in the first vessel 1221 on the sample preparation carousel 6805 as the first vessel 1221 is transported along the transport path. Additionally or alternatively, and as embodied herein, between 2 and 24 samples can be pooled in the first vessel 1221 on the sample preparation carousel 6805 as the first vessel 1221 is transported along the transport path.

Pool size can be selected as desired. For purpose of example and not limitation, a desired pool size can be supported by, for example, adjusting the pooled sample volume. For example, as the number of samples within a pool increases for a given pooled sample volume, the volume of liquid from each constituent sample of the pool is reduced. Accordingly, with larger pool sizes, the volume of the pooled sample can be increased so that a sufficient volume of each constituent sample can be included in the pooled sample. For purpose of example and as embodied herein, the size of pipette tips, lysis tubes, and/or amplification and detection vessels can be selected according to the desired pooled sample volume. Additionally or alternatively, additional system parameters can be adjusted. For example, the duration of a lockstep can be increased to allow additional time for movement of the at least one pipettor between movements of the sample transport, which can provide additional time for the pipettor to pool additional samples. Additionally or alternatively, the speed of the at least one pipettor can be increased. Additionally or alternatively, the at least one pipettor can include two or more pipette channels.

For purpose of example and illustration, an exemplary pooling process for pooling 24 samples on the sample preparation carousel 6805 is provided in Table 5.2 below.

TABLE 5.2

| Position(s) | Description | Running Time |
|---|---|---|
| L4 (Start) | Aspirate Sample 1 | 24 sec |
| | Dispense Sample 1 to L4 | |
| | Dispose of Tip | |
| | Aspirate Sample 2 | |
| | Dispense Sample 2 to L4 | |
| | Dispose of Tip | |
| L5 | Aspirate Sample 3 | 48 sec |
| | Dispense Sample 3 to L5 | |
| | Dispose of Tip | |
| | Aspirate Sample 2 | |
| | Dispense Sample 2 to L5 | |
| | Dispose of Tip | |
| L6 | Aspirate Sample 5 | 72 sec |
| | Dispense Sample 5 to L6 | |
| | Dispose of Tip | |
| | Aspirate Sample 6 | |
| | Dispense Sample 6 to L6 | |
| | Dispose of Tip | |
| L7 | Aspirate Sample 7 | 96 sec |
| | Dispense Sample 7 to L7 | |
| | Dispose of Tip | |
| | Aspirate Sample 8 | |
| | Dispense Sample 8 to L7 | |
| | Dispose of Tip | |
| L8 | Aspirate Sample 9 | 120 sec |
| | Dispense Sample 9 to L8 | |
| | Dispose of Tip | |
| | Aspirate Sample 10 | |
| | Dispense Sample 10 to L8 | |
| | Dispose of Tip | |
| L9 | Aspirate Sample 11 | 144 sec |
| | Dispense Sample 11 to L9 | |
| | Dispose of Tip | |
| | Aspirate Sample 12 | |
| | Dispense Sample 12 to L9 | |
| | Dispose of Tip | |
| L10 | Aspirate Sample 13 | 168 sec |
| | Dispense Sample 13 to L10 | |
| | Dispose of Tip | |
| | Aspirate Sample 14 | |
| | Dispense Sample 14 to L10 | |
| | Dispose of Tip | |
| L11 | Aspirate Sample 15 | 192 sec |
| | Dispense Sample 15 to L11 | |
| | Dispose of Tip | |
| | Aspirate Sample 16 | |
| | Dispense Sample 16 to L11 | |
| | Dispose of Tip | |
| L12 | Aspirate Sample 17 | 216 sec |
| | Dispense Sample 17 to L12 | |
| | Dispose of Tip | |
| | Aspirate Sample 18 | |
| | Dispense Sample 18 to L12 | |
| | Dispose of Tip | |
| L13 | Aspirate Sample 19 | 240 sec |
| | Dispense Sample 19 to L13 | |
| | Dispose of Tip | |
| | Aspirate Sample 20 | |
| | Dispense Sample 20 to L13 | |
| | Dispose of Tip | |
| L14 | Aspirate Sample 21 | 264 sec |
| | Dispense Sample 21 to L14 | |
| | Dispose of Tip | |
| | Aspirate Sample 22 | |
| | Dispense Sample 22 to L14 | |
| | Dispose of Tip | |
| L15 | Aspirate Sample 23 | 288 sec |
| | Dispense Sample 23 to L4 | |
| | Dispose of Tip | |
| | Aspirate Sample 24 | |
| | Aspirate Samples 1-22 from L15 | |
| | Dispense Samples 1-24 to L4 | |
| | Dispose of Tip | |

As embodied herein, sample preparation carousel 6805 can use a lockstep of 24 seconds. As shown in Table 5.2, the onboard pooling process can begin with the aspiration of a first sample for pooling. For purpose of example and as embodied herein, two samples can be added to the sample pool with every 24 second lockstep and duration of the pooling process for pooling 24 samples can be about 288 seconds. As embodied herein, the pooled sample can be dispensed into a new lysis tube at the sample dispense position L4 for further sample preparation, which can denote the end of the onboard pooling process. For example and not limitation and as embodied herein, 22 samples can be pooled in about 264 seconds. For example and not limitation and as embodied herein, 20 samples can be pooled in about 240 seconds. For example and not limitation and as embodied herein, 18 samples can be pooled in about 216 seconds. For example and not limitation and as embodied herein, 16 samples can be pooled in about 192 seconds. For example and not limitation and as embodied herein, 14 samples can be pooled in about 168 seconds. For example and not limitation and as embodied herein, 12 samples can be pooled in about 144 seconds. For example and not limitation and as embodied herein, 10 samples can be pooled in about 120 seconds. For example and not limitation and as embodied herein, 8 samples can be pooled in about 96 seconds. For example and not limitation and as embodied herein, 6 samples can be pooled in about 72 seconds. For example and not limitation and as embodied herein, 4 samples can be pooled in about 48 seconds. For example and as embodied herein, 24 samples, such as plasma or serum samples, can be pooled in about 5 minutes or less.

In accordance with another aspect of the disclosed subject matter, the disclosed methods and systems can include a pre-treatment process and onboard pooling can include onboard pooling of pre-treated samples. For purpose of example and as embodied herein, the pre-treatment process can include a whole blood lysis pre-treatment process, and onboard pooling can include pooling of whole-blood lysates.

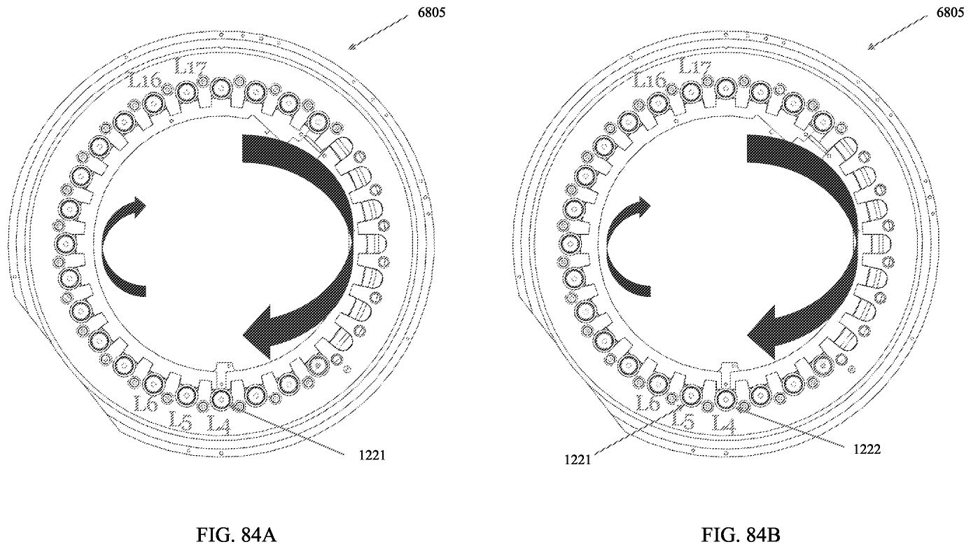

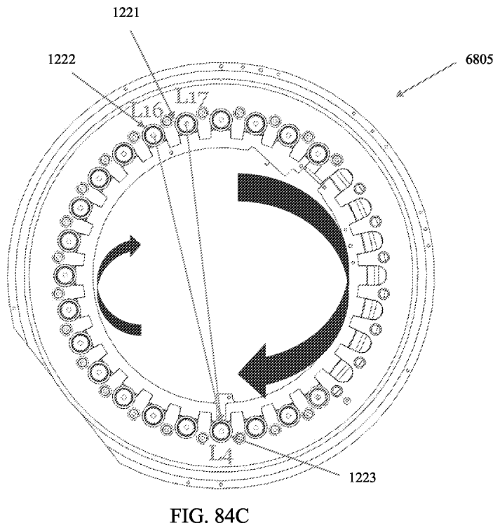

An exemplary pre-treatment process is described in more detail above. As embodied herein, the pre-treatment process can be performed on the sample preparation carousel 6805, and can include dispensing a sample into vessel on the sample preparation carousel 6805 and continually transporting the vessel with sample and reagents therein along a transport path defined between the sample dispense position and a capture and transfer position, with intermediate positions therebetween. For purpose of example and with reference to FIGS. 84A-84C, the pre-treatment process can include dispensing a first sample into a first vessel 1221 at the sample dispense position L4 prior to the sample preparation carousel transporting the first vessel 1221 to a first intermediate position L5. Pre-treatment can then be initiated for a second sample in a second vessel 1222 by dispensing a second sample into a second vessel 1222 at the sample dispense position L4 prior to the sample preparation carousel transporting the second vessel 1221 to the first intermediate position L5 and the first vessel 1221 to a second intermediate position L6. As embodied herein, vessels 1221 and 1222 can include reagents therein, such as for example a lysis buffer, and the first and second samples can be lysed as they are transported along the transport path towards the sample capture and transfer position L18. As embodied herein, the pre-treatment process can include mixing the sample and reagent at each intermediate position. Exemplary mixing systems are described further herein. As further embodied herein, the pre-treatment process includes lysing a sample for between about 3 and about 6 minutes.

In accordance with an aspect of the disclosed subject matter and as embodied herein, after a pre-treatment process, such as a whole-blood lysis pre-treatment process, onboard pooling can include pooling of whole blood lysates. For purpose of example and as embodied herein, a subset of pre-treated samples can be pooled on the sample preparation carousel 6805 and nucleic acid analysis can be being performed on the pooled sample. As embodied herein, pooling the pre-treated samples can include aspirating a first pre-treated sample from the first vessel 1221 at a first intermediate position and aspirating a second pre-treated sample from the second vessel 1222 at a second intermediate position. For purpose of example and with reference to FIG. 84C, the first sample can be aspirated from position L17 and the second sample can be aspirated from position L16. As embodied herein, the intermediate position from which the pre-treated samples are aspirated from can be selected based on aspects of the pre-treatment process. For example and as embodied herein, with a 24 second lockstep, the first and second samples can be transported to the L16 and L17 positions during the pre-treatment process to provide sufficient time to lyse the first and second samples.

Pooling the pre-treated samples can further include dispensing the first pre-treated sample and second pre-treated sample in a third vessel 1223 to combine and pool the first and second pre-treated samples. For example and as embodied herein, the first and second pre-treated samples can be dispensed into a third vessel 1223 at the sample dispense position L4 to combine and pool the pre-treated samples. As further embodied herein, dispensing and pooling the pre-treated samples at the sample dispense position L4 can initiate further sample preparation, such as an additional lysis process.

For purpose of example and illustration, an exemplary sample preparation process including a pre-treatment and pooling process for pre-treating and pooling 6 samples on the sample preparation carousel 6805 is provided in Table. 5.3 below.

TABLE 5.3

| Position(s) | Description | Running Time |
|---|---|---|
| L4 (Start) | Dispense Sample | 24 sec |
| | Dispose of Tip | |
| L5-L16 | Incubation and Mixing | 312 sec |
| L17 | Aspirate Sample 1 from L17 | 336 sec |
| | Aspirate Sample 2 from L16 | |
| | Aspirate Sample 3 from L15 | |
| | Aspirate Sample 4 from L14 | |
| | Aspirate Sample 5 from L13 | |
| | Aspirate Sample 6 from L12 | |
| | Dispense Samples 1-6 to L4 | |
| | Dispose of Tip | |

As embodied herein, sample preparation carousel 6805 can use a lockstep of 24 seconds. As shown in Table 5.3, the pre-treatment and pooling process can begin with the aspiration of a first sample for pre-treatment. For purpose of example and as embodied herein, Sample 1 can be incubated and mixed until Sample 1 reaches intermediate position L17, which corresponds to a pre-treatment time of about 336 seconds. As embodied herein, Samples 2-5 can be pre-treated for less time than Sample 1. For example, Sample 6 can be incubated and mixed until Sample 6 reaches intermediate position L14, which can correspond to a pre-treatment time of about 264 seconds. As embodied herein, the number of samples pre-treated and pooled can be selected based on the parameters of the pre-treatment process. For purpose of example and as embodied herein, pre-treatment times can range between about 3 and about 7 minutes for a lysis pre-treatment process for whole blood samples prior to pooling of the pre-treated samples. For example and as embodied herein, pre-treated samples 1-6 can be aspirated and dispensed into a new lysis tube at the sample dispense position L4 in a single 24 second lockstep, which can end the pre-treatment and pooling process. As embodied herein, the pre-treatment and pooling process for 6 samples of whole blood can be performed in about 6 minutes or less.

In accordance with another aspect of the disclosed subject matter, onboard pooling can be used in support of onboard deconstruction of a pooled sample. For purpose of example and as embodied herein, when nucleic acid(s) derived from one of a plurality of pathogens or infectious agents are determined to be present in a pooled sample, onboard deconstruction can be performed to identify which of the constituent samples includes the nucleic acid from the pathogen or infectious agent.

As embodied herein, onboard deconstruction of a pooled sample can include forming sub-pools, with each sub-pool being formed of a subset of the samples used to form the pooled sample. For purpose of example and as embodied herein, onboard as described herein can be used to prepare sub-pools according to the desired deconstruction strategy. As described above and with reference to the exemplary deconstruction strategies depicted in FIGS. 85A-85F, onboard deconstruction as embodied herein can include multiple rounds of sub-pool testing.

Additionally or alternatively, and as further embodied herein, onboard deconstruction can improve deconstruction efficiency and can, for example, reduce the number of tests needed to deconstruct a pooled sample and the time needed to deconstruct a pooled sample. For purpose of example and as embodied herein, the samples used to form a pooled sample can be stored onboard during nucleic acid analysis of the pooled sample. For example and as embodied herein, the samples used to form a pooled sample can be stored in loading area 102 during analysis of the pooled sample. Additionally or alternatively and as further embodied herein, when nucleic acid(s) derived from one of a plurality of pathogens or infectious agents are determined to be present in a pooled sample, the constituent samples used to form the pooled sample can be automatically transferred from the loading area 102 for further nucleic acid analysis.

For purpose of example and not limitation, the systems and methods for onboard pooling described herein can be used to form and test a pool of 96 samples. Additionally or alternatively, upon detection of the presence of a nucleic acid derived from one of a plurality of pathogens or infectious agents in the pooled sample of 96, the systems and methods for onboard deconstruction described herein can be used to deconstruct the pool of 96 samples. As embodied herein, a pool of 96 samples can be formed and deconstructed and a determination can be made as to whether donor material associated with each of the 96 constituent samples can be released for clinical use in less than about 4 hours from initial aspiration of the first constituent sample for nucleic acid analysis.

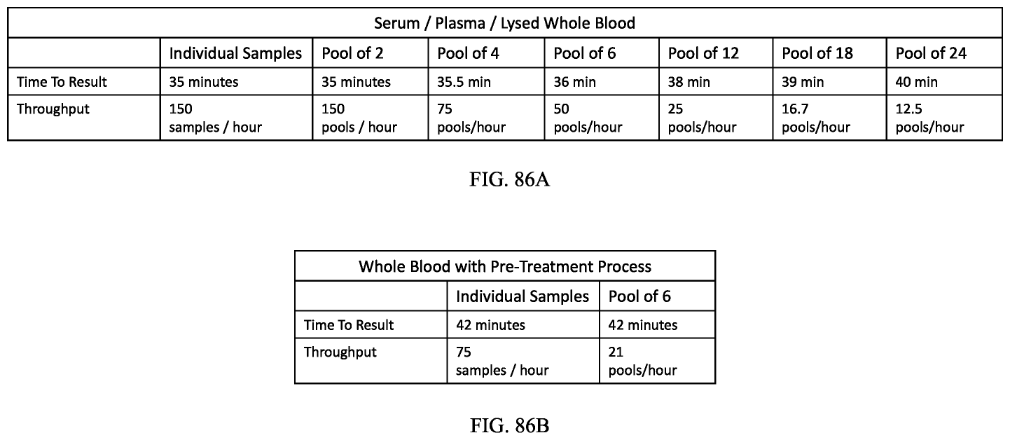

Additionally or alternatively, and as further embodied herein, onboard pooling can increase system throughput. For purpose of example and not limitation and as embodied herein, reference is made to FIGS. 86A and 86B, which depict exemplary throughput for exemplary sample types and processes for an exemplary HTNAT system such as the exemplary systems depicted in FIGS. 3, 62, 63, and 68A-D. FIG. 86A depicts exemplary throughput for serum, plasma, and/or lysed whole blood samples. For purpose of example and as embodied herein, HTNAT of serum, plasma, and/or lysed whole blood samples can include a lysis process, a wash and elution process, and an amplification and detection process as described herein. For purpose of example and not limitation and as embodied herein, a throughput of about 150 samples per hour can be achieved when processing individual samples. As embodied herein, a throughput of about 150 samples per hour can correlate to transferring one sample from the sample aspiration position 6892 to the sample preparation carousel for sample preparation every 24 second lockstep, leading to 150 sample aspirations per hour. As embodied herein, with a throughput of about 150 samples per hour, HTNAT systems as embodied herein can achieve 150 individual results per hour and up to 600 or more individual results per hour depending on, for example, whether a split eluate configuration and/or multiplex amplification and detection strategies are used.

With reference to FIG. 86A, onboard pooling in accordance with the disclosed subject matter can increase throughput. For purpose of example and as embodied herein, onboard pooling of two samples can include transferring two samples from the sample aspiration position 6892 to the sample preparation carousel for sample preparation every 24 second lockstep, leading to 300 sample aspirations per hour. For purpose of example and as embodied herein, onboard pooling in accordance with the disclosed subject matter can double the number of sample aspirations per hour. As embodied herein, the samples can be serum, plasma, and/or lysed whole blood samples, and HTNAT can include an onboard pooling process, a lysis process, a wash and elution process, and an amplification and detection process as described herein. With reference to FIG. 86A, when processing pools of two serum, plasma, and/or lysed whole blood samples, throughput can be about 150 pools/hr. As embodied herein, with a throughput of about 150 pools per hour, HTNAT systems as embodied herein can achieve 150 pool results per hour and up to 600 or more pool results per hour depending on, for example, whether a split eluate configuration and/or multiplex amplification and detection strategies are used. As embodied herein, each pool result, i.e., each result for a pooled sample, can facilitate a determination of the absence of one or more target nucleic acids in two or more individual samples. For example, if a pool result includes a determination of the absence of a target nucleic acid in the pool, the pool result can include a determination of the absence of target nucleic acid in two individual samples, i.e., using a pool size of 2. For example, using a pool size of 12 each pool result can include a determination of the absence of target nucleic acid in 12 individual samples. As embodied herein, onboard pooling in accordance with the disclosed subject matter can facilitate determinations of the absence of target nucleic acids for more individual samples per hour as compared to high-throughput analysis of individual samples without pooling.

For purpose of example and without limitation, in accordance with an aspect of the disclosed subject matter between about 240 and about 340 samples can be transferred from the sample aspiration position 6892 to the sample preparation carousel per hour.

FIG. 86B depicts exemplary throughput for whole blood samples including a pre-treatment process as described herein. For purpose of example and as embodied herein, HTNAT of whole blood samples can include a pre-treatment process, a lysis process, a wash and elution process, and an amplification and detection process as described herein. For purpose of example and as embodied herein, a throughput of about 75 samples per hour can be achieved when processing individual samples. As embodied herein, processing individual whole blood samples can include transferring one sample from the sample aspiration position 6892 to the sample preparation carousel for sample preparation every other 24 second lockstep, leading to about 75 sample aspirations per hour. As embodied herein, with a throughput of about 75 samples per hour, HTNAT systems as embodied herein can achieve 75 individual results per hour and up to 300 or more individual results per hour depending on, for example, whether a split eluate configuration and/or multiplex amplification and detection strategies are used.

With reference to FIG. 86B, onboard pooling in accordance with the disclosed subject matter can increase throughput when processing whole blood samples. For purpose of example and as embodied herein, onboard pooling of six whole blood samples can include transferring one sample from the sample aspiration position 6892 to the sample preparation carousel for five out of every six 24 second locksteps, leading to about 125 sample aspirations per hour. As embodied herein, high-throughput analysis of the whole blood samples can include a pre-treatment process, an onboard pooling process, a lysis process, a wash and elution process, and an amplification and detection process as described herein. With reference to FIG. 86B, when processing pools of six whole blood samples, throughput can be about 21 pools/hr. As embodied herein, with a throughput of about 21 pools per hour, HTNAT systems as embodied herein can achieve 21 pool results per hour and up to about 84 or more pool results per hour depending on, for example, whether a split eluate configuration and/or multiplex amplification and detection strategies are used. As embodied herein, each pool result can facilitate a determination of the absence of one or more target nucleic acids in two or more individual samples. For example, if a pool result includes a determination of the absence of a target nucleic acid in the pool, the pool result can include a determination of the absence of target nucleic acid in 6 individual samples, i.e., using a pool size of 6.

Systems and methods for onboard pooling as embodied herein can provide numerous advantages. For purpose of example and not limitations, the disclosed systems and methods for onboard pooling matter can increase testing throughput and can reduce the number of tests required. For example, as compared with HTNAT of individual samples, onboard pooling as described herein can increase the number of individual samples tested per hour. Additionally or alternatively, using onboard pooling and HTNAT to screen for pathogens or infectious agents can provide increased efficiency for the release of donor material associated with the pooled sample for clinical use and can reduce the total number of tests required to assess a given number samples, particularly when screening for pathogens or infectious agents with low prevalence in the sample population. Additionally or alternatively, the disclosed systems and methods for onboard pooling can increase laboratory throughput by reducing laboratory bottlenecks associated with conventional pooling on separate liquid handlers or poolers.

Additional benefits of the disclosed systems and methods for onboard pooling can include, for example and not limitation, reducing the number of liquid handlers or poolers needed in a laboratory to support high-throughput testing. For purpose of example and not limitation, by pooling samples onboard, the number of liquid handlers or poolers in a laboratory can be reduced, which can reduce costs (e.g., fewer pieces of equipment needed) and can free up laboratory floor space for alternative uses.

Additional benefits of the disclosed systems and methods for onboard pooling can include, for example and not limitation, pool size flexibility. For example, when pooling on conventional liquid handlers or poolers pool sizes are generally selected according to the number of pipette channels on the liquid handler or pooler. In accordance with an aspect of the disclosed subject matter, pool size can be selected as desired. For purpose of example and not limitation, pool size can be selected based on the prevalence of pathogens or infectious agents in the sample population.

Additional benefits of the disclosed systems and methods for onboard pooling can include, for example and not limitation, efficient use of laboratory floor space. For example, and as embodied herein, systems and methods for onboard pooling and HTNAT can use system resources for multiple processes. For example and as embodied herein, the sample preparation carousel can be used for pooling, pre-treatment, and lysis processes. By using system components for multiple processes, the floor space required for high-throughput analysis can be reduced as compared to systems and methods which may require separate systems and floor space for each process. Additionally or alternatively, and as described above, additional equipment, such as poolers, can be reduced.

Additional benefits of the disclosed systems and methods for onboard, for example and not limitation, reduced testing error. For purpose of example and not limitation, automated onboard deconstruction as described herein can reduce the manual steps and opportunities for error which can be associated with conventional deconstruction methods.

### 6.1.4 Nucleic Acid Wash & Elute Systems

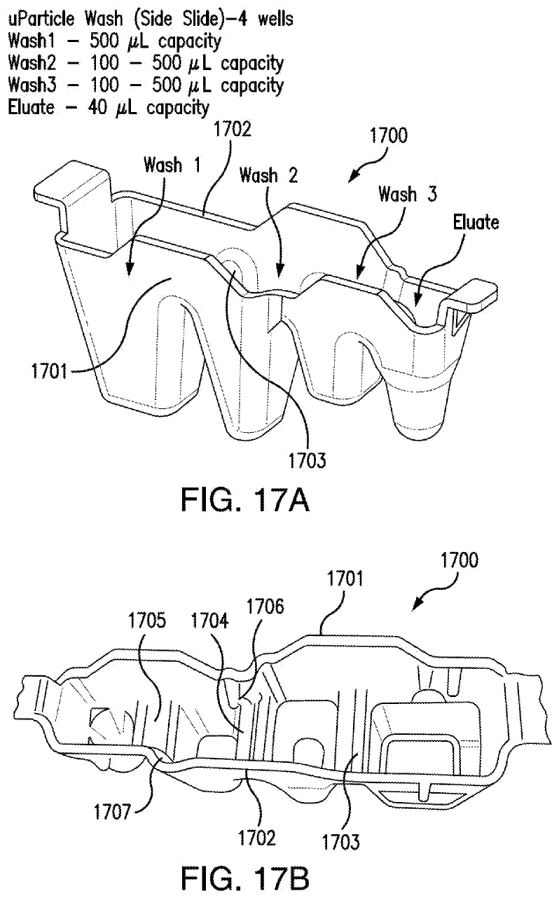

FIG. 65 is a diagram illustrating an exemplary wash and elute subsystem 6530 according to the disclosed subject matter. Generally, the Wash and Elute system washes the sample to eliminate undesired material from the microparticles and then elutes the captured material, e.g., nucleic acids, from the microparticles. In certain embodiments, as shown in FIG. 65, the sample transport 6530 is a rotatable carousel 6531 having positions to hold wash vessels 6540. The wash vessels 6540 hold wash buffer and elution buffer during the wash and elution process. FIG. 65 comprises 24 positions (clockwise around the central carousel), which are identified as wash 1 (“W1”) through wash 24 (“W24”). In the embodiment of FIG. 65, there are generally 12 radially oriented slots 6535, with each slot having two positions for holding a wash vessel 6540. For example, position W1 and W13 are generally located on the same slot 6535. In the embodiment of FIG. 65, a plurality of wash vessels 6540 are shown at each of the 24 positions. Not all positions on the carousel 6531 are used in the sample washing and elution process in that some position are used to load the wash vessels onto the carousel, dispense wash buffer or remove the wash vessel. The wash vessels each define a plurality of wells (P1-P4), as shown in one embodiment in FIG. 17, for the wash and elute process. Embodiments of the present disclosure can comprise greater or fewer than 24 positions, depending on alternative system configurations or the desired capacity or throughput of the system, and the individual positions need not be arranged as illustrated, but rather can be arranged in carousel, linear, or essentially any other orientation compatible with the throughput requirements of the instant disclosure

The below table, Table 6, depicts exemplary times and operations regarding the wash and elution process (also referred to herein as the wash process). As shown in this exemplary embodiment using a lockstep of 24 seconds, the sample processing time for the wash and elute process is 480 seconds, or 8 minutes. When not taking into consideration the transfer of the microparticles from the lysis process (e.g., position W3 in Table 6), the sample processing time for the wash and eluate process (also referred to herein as the washing process) is 456 seconds, or 7.6 minutes.

TABLE 6

| | | Sample Process |
|---|---|---|
| Pos. | Function | Time (Seconds) |
| W1 | Load Wash Vessel in Carousel | |
| W2 | Dispense Wash 1 | |
| | (500 μl +/− 5%) | |
| | Dispense Wash2 | |
| | (100-500 μl +/− 5%) | |
| | Dispense Wash3 | |
| | (100-500 μl +/− 5%) | |
| W3 | Pickup Transfer Tip | 24 |
| | Capture μParticles | |
| | Transfer μParticles | |
| W4-7 | Incubation (40° C.) | 96 |
| | Mixing Wash 1 (96 seconds) | |
| | 88 Mixes (wide field) | |
| W8 | Transfer μParticles to Wash 2 | 24 |
| W9 | Mixing Wash 2 (24 seconds) | 24 |
| | 12 Mixes (wide field) | |
| W10 | Transfer μParticles to Wash 3 | 24 |
| | Pre-heat Elution well to 70° C. | |
| W11 | Mixing Wash 3 (24 seconds) | 24 |
| | 12 Mixes (wide field) Dispense 70° C. | |
| | Elution Buffer (60 μl +/− 5%) | |
| | Pre-heat Elution well to 70° C. | |
| W12 | Transfer μParticles to Elution | 24 |
| | Pre-heat Elution well to 70° C. | |
| W13-20 | Incubation (80° C.) | 192 |
| | Mixing Elution (192 seconds) | |
| | 64 Mixes | |
| W21 | Transfer μParticles to Wash 3 | 24 |
| | Cool Elution well to 40° C. | |
| W22 | Aspirate Eluent | 24 |
| | (42 μl +/− 5%) | |
| | Dispense Eluent to Amp Vessel 1 | |
| | (20 μl +/− 5%) | |
| | Dispense Eluent to Amp Vessel 2 | |
| | (20 μl +/− 5%) | |
| | Wash Probe | |
| W23 | Aspirate Wash contents to Waste | |
| W24 | Transfer Wash Vessel to Waste | |
| | Indexing | |

In the exemplary embodiment illustrated in FIG. 65, W1 corresponds to a transfer position where the wash vessel is transferred to the carousel from, e.g., a commodity loading area. Transfer of wash vessels to this position can be accomplished via known methods in the art, e.g., sideways shuffle strategies and/or “Pick & Place” strategies. In certain embodiments, the plurality of wash vessels 6440 identified in FIG. 65 can be arranged as illustrated in FIG. 17. For example, but not by way of limitation, positions P1, P2, P3, and P4 described below can correspond to Wash 1, Wash 2, Wash 3, and Eluate of FIG. 17. In certain aspects, the time to perform the procedure corresponding to exemplary position W1 is not considered in the calculation of TTR. In certain embodiments, the time to perform the procedure corresponding to exemplary position W1 is not considered in the calculation of the duration of the washing process.

Exemplary position W2 corresponds to a dispensing position to dispense wash solution into wells P1-P3. Dispensing of wash solution into well P1 can be accomplished, e.g., via direct plumb from a bulk reservoir. In certain embodiments, the wash solution of well P1 is dispensed at a volume of 500 μl (+/−5%) although other volumes are contemplated within the instant disclosure. In certain embodiments, the wash solution of well P1 comprises about 2.5M to about 4.7M GITC, about 2% to about 10% Tween-20, and a pH of about 5.5 to about 8.0. In certain embodiments, e.g., with respect to plasma and serum samples, the wash solution of wells of P1 comprises about 3.13 M GITC, 6.7% Tween-20, 100 mM Tris, and a pH of about 7.8. In certain embodiments, the wash solution of wells of P1 comprises about 4.7M GITC, about 10% Tween-20, and a pH of about 7.8. In certain embodiments, e.g., with respect to whole blood samples, the wash solution of wells P1 comprises about 3.5M GITC, about 2.5% Tween-20, and a pH of about 6.0. Position W2 also corresponds to a remainder fluid dispensing position into the well array and the dispensing of elution buffer. At W2, additional wash solution is dispensed into well P2, wash solution is dispensed into well P3 and elution buffer is dispensed into well P4. In certain embodiments, the Elution Buffer comprises about 5 mM to about 10 mM Phosphate and a pH of about 7.5 to about 9.0. In certain aspects, the time to perform the procedures corresponding to exemplary position W2 is not considered in the calculation of TTR. In certain embodiments, the time to perform the procedure corresponding to exemplary position W2 is not considered in the calculation of the duration of the washing process.

Exemplary position W3 corresponds to a microparticle transfer position. In certain embodiments, transfer is accomplished via retraction of the capture magnet and shaking of the tip to deposit the microparticles into well P1 in W3. In certain aspects, the time to perform the procedures corresponding to exemplary position W3 is not considered in the calculation of TTR. In certain embodiments, the time to perform the procedure corresponding to exemplary position W3 is not considered in the calculation of the duration of the washing process.

In certain embodiments, the time to perform the procedures corresponding to exemplary positions W1, W2 and W3 is not considered in the calculation of TTR. In certain embodiments, the time to perform the procedure corresponding to exemplary positions W1, W2 and W3 is not considered in the calculation of the duration of the washing process.

Exemplary positions W4-W7 correspond to incubation and mixing positions. Incubation and mixing at positions W4-W7 can incorporate the use of resistive heaters, mixing via top and/or bottom magnets, e.g., moving permanent magnets, as well as carousel movement, pop-up mixers, and/or electro-magnets (the use of stationary electromagnet-based mixing of magnetic microparticles is described in detail in Example 2, below). In certain embodiments, positions W4-W7 will employ a heater, e.g., a resistive heater, to heat the lysis sample to about 37° C. In certain embodiments, W4-W7 employ incubation in Wash 1 for about 96 seconds, or about 1.6 minutes (4 locksteps at 24 seconds each). In certain embodiments, position W8 involves transfer of the microparticles into the wash solution at well P2 for Wash 2 and mixing. In certain embodiments, the first wash step (i.e., Wash 1) can include the incubation and mixing positions (e.g., exemplary positions W4-W7) and the transfer of the microparticles into well P2 (e.g., exemplary position W8) such that the first wash step has a duration of about 120 seconds, or about 2 minutes (5 locksteps at 24 seconds each). In certain embodiments, the first wash step (i.e., Wash 1) does not include the transfer of the microparticles into well P2 (e.g., exemplary position W8) such that the first wash step has a duration of about 96 seconds, or about 1.6 minutes (4 locksteps at 24 seconds each). In certain embodiments, the transfer can be accomplished using moveable magnets above the wells to slide the microparticles across the internal surface of the seal to collect, transfer and release the microparticles. In some embodiments, transfer can be accomplished using moveable magnets below the wells to slide the microparticles within internal channels at the bottom of wells to collect, transfer and release the microparticles. In certain embodiments stationary electro-magnets, e.g., selectively turning on/off adjacent magnets to achieve magnetic particle movement can be employed. Other methods of transferring microparticles such as in inverse particle processing can also be used.

In certain embodiments, position W9 comprises incubation and mixing in the wash solution at well P2 for about 24 seconds (1 lockstep at 24 seconds). In certain embodiments, Wash 2 is water. In certain embodiments, mixing is accomplished via carousel movement, a pop-up mixer, and/or electro-magnets. In certain embodiments, exemplary position W10 involves transfer of the microparticles into the wash solution at well P3 for Wash 3 and mixing. In certain embodiments, the incubation in Wash 2, i.e., the second wash step, can include the incubation and mixing positions (e.g., exemplary position W9) and the transfer of the microparticles into the wash solution at well P3 for Wash 3 and mixing (e.g., exemplary position W10) such that Wash 2 is for about 48 seconds (2 locksteps at 24 seconds each). In certain embodiments, the second wash step does not include the transfer of the microparticles into well P3 (e.g., exemplary position W10) such that the second wash step is for about 24 seconds (1 lockstep at 24 seconds each). In certain embodiments, the transfer can be accomplished using moveable magnets above the wells to slide the microparticles across the internal surface of the seal to collect, transfer and release the microparticles.

In certain embodiments, position W11 comprises incubation and mixing in the wash solution in well P3 for about 24 seconds (1 lockstep at 24 seconds). In certain embodiments, the wash solution at well P3 is water. In certain embodiments, mixing is accomplished via carousel movement, a pop-up mixer, and/or electro-magnets. In certain embodiments, mixing is accomplished using an offline orbital mixer at 1500 rpm.

In certain embodiments, positions W12-W20 correspond to transfer and incubation positions where the sample is incubated within the elution buffer to remove the target from the microparticles. For example, but not limitation, W12 is a transfer position where the microparticles are transferred, e.g., via movable permanent magnets or stationary electro-magnets, into the elution buffer at well P4. In certain embodiments, the duration of Wash 3 (i.e., the third wash step) can include the time at the incubation and mixing positions (e.g., exemplary position W11) and the transfer of the microparticles into well P4 (e.g., exemplary position W12) such that Wash 3 is for about 48 seconds (2 locksteps at 24 seconds each). In certain embodiments, the duration of Wash 3 (i.e., the third wash step) does not include the transfer of the microparticles into well P4 (e.g., exemplary position W12) such that Wash 3 (i.e., the third wash step) is for about 28 seconds (1 lockstep at 24 seconds each).

Strategies for elution of nucleic acids from solid supports, e.g., microparticles, are known in the art. For example, but not by way of limitation, nucleic acids, e.g., RNA and/or DNA, can be eluted by contacting the solid supports to which the nucleic acids are bound, e.g., microparticles, with an elution buffer with or without concurrent heating. In certain embodiments, the elution buffer comprises phosphate (e.g., inorganic phosphate or organophosphate) at a concentration of 1 to 10 mM (e.g., 1, 2, 3, 4, 5, 6, 7, 8, 9, or 10 mM). In some embodiments, the phosphate concentration is chosen to preferentially bind and/or elute the nucleic acid, e.g., DNA or RNA. In one embodiment, the wash vessel on the outer portion 137 of the wash and eluate carousel 131 at position W12 is moved radially inward to an inner portion 139 within the slot 135 as it is transferred to W13.

Heating during positions W13-W20 incubation can be accomplished using, e.g., resistive convection heaters. For example and as embodied herein, the resistive convection heaters can use forced air to transfer heat from one or more resistive heaters to the elution well. In certain embodiments, the incubation in the elution buffer at P4 (i.e., the elution step) extends for about 192 seconds, or about 3.2 minutes (e.g., 8 locksteps at 24 seconds each). At the completion of the incubation, the microparticles are transferred back into the wash solution of well P3, leaving substantially microparticle-free eluate in well P4. For example, such a transfer can occur at exemplary position W21 (1 lockstep at 24 seconds). At W22, approximately 42 μl (+/−5%) of eluent is aspirated (e.g., after an about 12 second delay to cool eluent to 40° C.), and half of the eluent is dispensed into one or more amplification vessels. In certain embodiments, the duration of the elution step can include the time at the incubation positions (e.g., exemplary positions W13-W20), the transfer of the microparticles into well P3 (e.g., exemplary position W21) and the aspiration and transfer of the eluate into an amplification vessel in the amplification and detection system (e.g., exemplary position W22) such that the elution step is for about 240 seconds (10 locksteps at 24 seconds each). In certain embodiments, the duration of elution step does not include the transfer of the microparticles into well P3 (e.g., exemplary position W21) and does not include the aspiration and transfer of the eluate into an amplification vessel in the amplification and detection system (e.g., exemplary position W22) such that the elution step is for about 192 seconds (8 locksteps at 24 seconds each). In certain embodiments, the duration of elution step does not include the aspiration of the eluate into an amplification vessel in the amplification and detection system (e.g., exemplary position W22) such that the elution step is for about 216 seconds (9 locksteps at 24 seconds each).

Exemplary position W23 corresponds to an aspiration of the wash contents to waste. Exemplary position W24 corresponds to the transfer of the wash vessel to waste. In certain aspects, the time to perform the procedures corresponding to exemplary positions W23 and W24 is not considered in the calculation of TTR. In certain aspects, the time to perform the procedures corresponding to exemplary positions W23 and W24 is not considered in the calculation of TTR. For example, but not by way of limitation, the time to perform the procedures corresponding to exemplary positions W23 and W24 is not considered in the calculation of the duration of the wash process.

In the above exemplary system, where a lockstep of 24 seconds is used, the sample is in the wash process for 20 locksteps (e.g., exemplary positions W3-W22), which results in a total processing for the wash process of about 480 seconds, or about 8 minutes. When not taking into consideration the transfer of the microparticles from the lysis process, the sample is in the wash process for 19 locksteps (e.g., exemplary positions W4-W22), which results in a total processing for the wash and eluate process of about 456 seconds, or about 7.6 minutes.

In certain embodiments, the processing time of the sample for the washing process starts with incubation in the first wash well (e.g., exemplary position W4) and ends with dispensing the eluate into a vessel in the amplification and detection system (e.g., exemplary position W22). In certain embodiments, the total sample processing time in the wash process can be from about 5 minutes to about 9 minutes, from about 5 minutes to about 8 minutes, or from about 7 minutes to 8 minutes. In certain embodiments, the total sample processing time in the wash process can be about 5 minutes, about 6 minutes, about 7 minutes, about 8 minutes, or about 9 minutes. In particular embodiments, the total sample processing time in the wash process can be about 456 seconds, or about 7.6 minutes or about 336 seconds, or 5.6 minutes.

The wash process can include a first wash step (e.g., Wash 1), a second wash step (e.g., Wash 2), a third wash step (e.g., Wash 3), and an elution step. In certain aspects, the first wash step starts at the start of incubation in the first wash well (e.g., exemplary positions W4-W7) and ends with the transfer of the microparticles to the second wash well (e.g., exemplary position W8). In certain embodiments, the first wash step starts with the start of incubation in the first wash well and ends with the end of incubation in the first wash well (e.g., exemplary positions W4-W7). In certain embodiments, the first wash step can be completed in about 0.5 minutes to about 3 minutes, about 1 minute to about 3 minutes, or about 1.5 minutes to about 3 minutes. In certain embodiments, the first wash step is about 0.5 minutes, about 1 minute, about 2 minutes, or about 3 minutes. In certain embodiments, the first wash step is about 96 seconds, or about 1.6 minutes or about 120 seconds, or about 2 minutes. In certain aspects, the second wash step starts at the start of incubation in the second wash well (e.g., exemplary position W9) and ends with the transfer of the microparticles to the third wash well (e.g., exemplary position W10). In certain embodiments, the second wash step starts with the start of incubation in the second wash well and ends with the end of incubation in the second wash well (e.g., exemplary position W9). In certain embodiments, the second wash step can be completed in about 0.1 minutes to about 2 minutes, about 0.4 minutes to about 1 minute, or about 0.4 minutes to about 0.8 minutes. In certain embodiments, the second wash step is about 0.1 minutes, about 0.4 minutes, about 0.8 minutes, about 1 minute, about 1.5 minutes, or about 2 minutes. In particular embodiments, the second wash step is about 48 seconds, or about 0.8 minutes or about 24 seconds, or about 0.4 minutes. In certain aspects, the third wash step starts at the start of incubation in the third wash well (e.g., exemplary position W11) and ends with the transfer of the microparticles to the elution well (e.g., exemplary position W12). In certain embodiments, the third wash step starts with the start of incubation in the third wash well and ends with the end of incubation in the first wash well (e.g., exemplary position W11). In certain embodiments, the third wash step can be completed in about 0.1 minutes to about 2 minutes, about 0.4 minutes to about 1 minute, or about 0.4 minutes to about 0.8 minutes. In certain embodiments, the third wash step is about 0.1 minutes, about 0.4 minutes, about 0.8 minutes, about 1 minute, about 1.2 minutes, or about 2 minutes. In particular embodiments, the second wash step is about 48 seconds, or about 0.8 minutes or about 24 seconds, or about 0.4 minutes. In certain aspects, the elution step starts with the start of incubation and mixing of the elution (e.g., exemplary positions W13-W20) and ends with aspirating the eluate and transferring the eluate to a vessel in the amplification and detection system (e.g., exemplary position W22). In certain embodiments, the elution step starts with the start of incubation and mixing of the elution and ends with the end of incubation and mixing of the elution (e.g., exemplary positions W13-W20). In certain embodiments, the elution step can be completed in about 2 minutes to about 6 minutes, about 3 minutes to about 5 minutes, or about 3 minutes to about 4 minutes. In certain embodiments, the elution step is about 2 minutes, about 2.4 minutes, about 3 minutes, or about 4 minutes. In particular embodiments, the elution step is about 192 seconds, or about 3.2 minutes or about 240 seconds, or about 4 minutes.

For TTR calculations, the processing time of the sample in the wash process does not include transferring the wash vessel to the carousel (e.g., exemplary position W1), dispensing the wash solution into wells P1-P3 (e.g., exemplary position W2), transferring the microparticles into well P1 (e.g., exemplary position W3), aspirating the wash contents to waste (e.g., exemplary position W23), and/or transferring the wash vessel to waste (e.g., exemplary position W24).

In certain embodiments more than three washes can be employed in the context of the instant disclosure. Moreover, the systems of the present disclosure a capable of processing distinct samples, e.g., serum/plasma samples and whole blood samples, including the with respect to the above-described steps or any step described below, such that individual samples can be handled in distinct manners in a single batch. For example, but not by way of limitation, individual samples in a batch can be incubated for longer or shorter periods and/or can be heated or cooled at distinct temperatures.

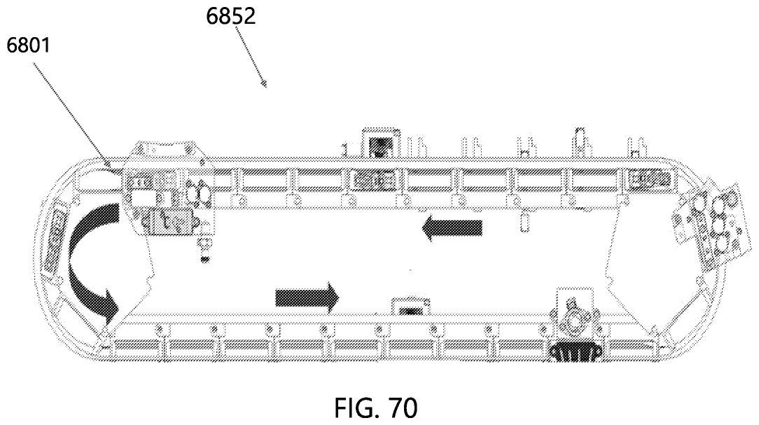

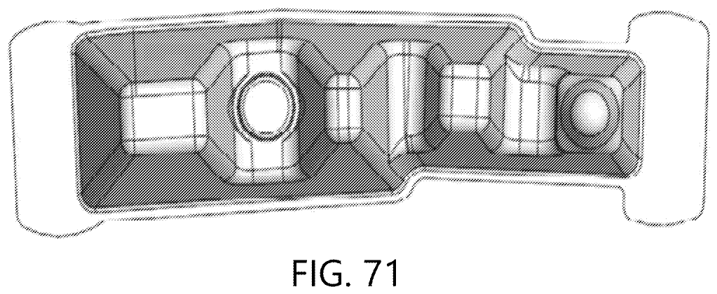

FIG. 70 depicts an exemplary wash track 6801 for practicing the inventions disclosed in accordance with the exemplary system depicted in FIGS. 68A-68D. As embodied herein, the wash track 6801 can be in a racetrack shape, rotating in a counterclockwise direction. For purpose of illustration not limitation, the racetrack shape of the wash track 6801 can save the footprint of the overall system. For purpose of illustration not limitation, the wash track 6801 includes 24 track positions. For each track position, the wash can be conducted in a wash vessel for up to 3 times. The wash vessel can be exemplary embodiments according to the subject matter disclosed in FIGS. 17A-17B. Alternatively, or additionally, the wash vessel can be an exemplary embodiment as illustrated in FIGS. 42A-42D. In certain embodiments, the wash vessel can be an exemplary embodiment as illustrated in FIG. 71, wherein the wash vessel can have a curved design along the horizontal direction of the vessel, and can have a curvature similar to a curvature of the wash track 6801.

Operational steps involving a stationary electromagnet-based capture of magnetic particles include but are not limited to: mixing; washing; and transfer of the magnetic particles. Existing methods of mixing and washing magnetic particles generally rely on mechanical agitation and magnetic mixing using moving permanent magnets. In certain embodiments, however, such mixing, washing, and/or transfer can be performed with a system incorporating at least one stationary electromagnet-based capture of magnetic particles, e.g., as exemplified in FIGS. 42A-42D. For example, but not limitation, and as embodied in FIG. 42A, magnetic microparticles 4202 can be present in a first vessel of a plurality of vessels 4206. Alternatively, or additionally, an electromagnet 4204 can be used to capture the magnetic microparticles to the wall of the vessel 4206, as illustrated in FIG. 42B. Alternatively, or additionally, movement of the electromagnet 4204 to a second vessel induces the transfer of the magnetic microparticles 4202 from the first vessel to the second vessel, as illustrated in FIG. 42C. Alternatively, or additionally, elimination of the current to the electromagnet 4204 once the magnetic microparticles 4202 have transferred to the second vessel allows for release of the magnetic microparticles 4202 as illustrated in FIG. 42D. Alternatively, or additionally, one or more of the distinct mixing or washing positions identified herein can be accomplished using a stationary electromagnet-based capture of magnetic particles. In addition, certain embodiments described herein can be performed with a system incorporating at least one stationary electromagnet-based capture of magnetic particles in connection with one or more transfer operations at one or more of the transfer positions within the systems described herein. As will be appreciated by those of skill in the art, the specific number, orientation, and operational assignment (e.g., mixing, washing, transfer, or elution) of the individual positions can be modified as desired and yet remain within the scope of the present disclosure.

The present disclosure contemplates the use, in certain embodiments, of stationary electromagnets in a variety of positions to accomplish the appropriate mixing, washing, and transfer operations. For example, but not by way of limitation, electromagnets can be positioned at right side, left side, and/or bottom side of a well. In addition, electromagnets can be positioned with one side higher than the other. The stationary electromagnets of the present disclosure can also be used in conjunction with a variety of well formats known in the art.

In certain embodiments, opposing electromagnets can be arranged to alternately attract magnetic particles to the opposite sides of a wash well. However, the position, timing, power, and sequence of the electromagnet activations is entirely flexible. For example, but not by way of limitation, stationary electromagnets and be used to partially collect magnetic particles and then allow them to drop to the bottom of the well. Alternatively, or additionally, magnetic particles can be completely collected alternately on the sides of a well to facilitate mixing, washing, and/or transfer. In addition, magnetic particles can be slowly collected with lower power or magnetic particles can be more rapidly collected with higher power.

Distinct types of electromagnets are also contemplated for use in connection with the embodiments disclosed herein. For example, while DC electromagnets can be used, because DC electromagnets can magnetize magnetic particles, AC electromagnets can also be used in conjunction with or in place of DC electromagnets to avoid creating residual magnetism of the magnetic particles by varying the switching frequency.

The use of electromagnets in addition to or in lieu of permanent magnets (or other mixing, washing, or transfer strategies) provides several advantages. Mixing or transfer operations during sample preparation typically requires permanent magnets to be moved in and out of range of the magnetic particles. The use of electromagnets can eliminate motion mechanisms by simply turning on and off the electromagnets. In addition, magnetic particles can be moved from one well to another by successively turning on and off adjacent magnets within an array. Incorporating a stationary electromagnet-based particle capture approach can also eliminate one or more disposable per test, thus reducing the amount of solid waste being generated. The use of stationary electromagnets also eliminates the need for specific volume requirements and disposable coverings for the wells or moving permanent magnet when transferring magnetic particles from one well to another. The instant strategy can also transfer magnetic particles between wells using moving electromagnets adjacent to the side of the wash vessel, but not touching the magnetic particles or liquid within wells, minimizing magnetic particle loss and liquid carryover from source to destination wells, thus maximizing assay performance.

In some embodiments, transfer can be accomplished using moveable permanent magnets below the wells to slide the microparticles internal channels at the bottom of wells to collect, transfer and release the microparticles. In certain embodiments stationary electro-magnets, e.g., selectively turning on/off adjacent magnets to achieve magnetic particle movement can be employed. Other methods of transferring microparticles such as in inverse particle processing can also be used.

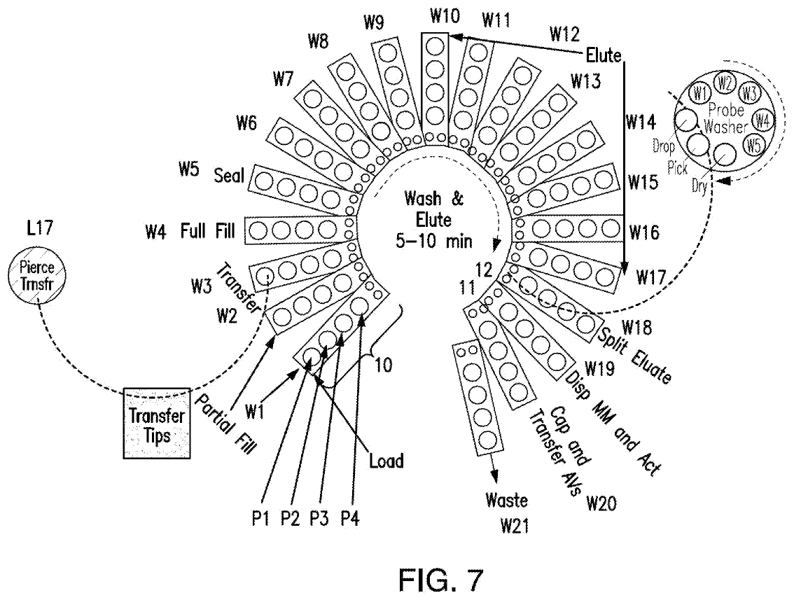

An alternative wash and elute system is exemplified in FIG. 7. For example, but not by way of limitation, FIG. 7 depicts a system that comprises 21 positions (clockwise around the central carousel), which are identified as Wash 1 (“W1”) through Wash 21 (“W21”). In the exemplary embodiment of FIG. 7, a plurality of well plates (10) are shown at each of the 21 positions. The well plates each define a plurality of wells (#P1-P4) for the wash and elute process. Embodiments of the present disclosure can comprise greater or fewer than 21 positions, depending on alternative system configurations or the desired capacity or throughput of the system, and the individual positions need not be arranged as illustrated, but rather can be arranged in carousel, linear, or essentially any other orientation compatible with the throughput requirements of the instant disclosure.

In the exemplary embodiment illustrated in FIG. 7, W1 corresponds to a transfer position where the wash vessel is transferred to the carousel from, e.g., a commodity loading area. Transfer of wash vessels to this position can be accomplished via known methods in the art, e.g., sideways shuffle strategies and/or “Pick & Place” strategies.

Exemplary position W2 corresponds to a partial dispensing position to dispense wash solution into well P1. Dispensing of wash solution into well P1 can be accomplished, e.g., via direct plumb from a bulk reservoir. In certain embodiments, the wash solution of well P1 is dispensed at a volume of 250p (+/−5%) although other volumes are contemplated within the instant disclosure, e.g., 500 μl (+/−5%). In certain embodiments, the wash solution of well P1 comprises about 2.5M to about 4.7M GITC, about 2% to about 10% Tween-20, and a pH of about 5.5 to about 8.0. In certain embodiments, e.g., with respect to plasma and serum samples, the wash solution of well P1 comprises about 4.7M GITC, about 10% Tween-20, and a pH of about 7.8. In certain embodiments, e.g., with respect to whole blood samples, the wash solution of well P1 comprises about 3.5M GITC, about 2.5% Tween-20, and a pH of about 6.0. In certain embodiments, e.g., where the lysis tube has not yet been pierced, the system can also perform the following: pickup of a transfer tip, e.g., via “Pick & Place” from loadable rack; piercing of the Lysis Tube, e.g., via pushing of the transfer tip through the Lysis Tube seal (and twisting, if desired, to increase hole clearance); capture of microparticles, e.g., via magnetic capture against structural elements of the transfer tip. In certain embodiments, such structural elements can include but are not limited to fins, e.g., for magnetic capture of microparticles between the fins.

Exemplary position W3 corresponds to a microparticle transfer position. In certain embodiments, transfer is accomplished via retraction of the capture magnet and shaking of the tip to deposit the microparticles into well P1 in W3.

Exemplary position W4 corresponds to a remainder fluid dispensing position into the well array and the dispensing of elution buffer. At W4, additional wash solution is dispensed into well P1, wash solution is dispensed into well P2, wash solution is dispensed into well P3 and elution buffer is dispensed into well P4. Dispensing of the wash solution can be accomplished, e.g., via direct plumb from a bulk reservoir or “Sip & Spit” from a reagent container. In certain embodiments, the remainder the wash solution of well P1 is dispensed at a volume of 250p (+/−5%), 100-500 μl (+/−5%), and/or 60 μl (+/−5%) although other volumes are contemplated within the instant disclosure. In certain embodiments, the Elution Buffer comprises about 5 mM to about 10 mM Phosphate and a pH of about 7.5 to about 9.0.

Exemplary position W5 corresponds to a wash vessel sealing position. Sealing of a wash vessel at this position can be accomplished via known methods in the art, e.g., heat stake tape, press on caps, and PSA tape.

Exemplary positions W6-W8 correspond to incubation and mixing positions. Incubation and mixing at positions W6-W8 can incorporate the use of resistive heaters, mixing via top and/or bottom magnets, e.g., permanent magnets, as well as carousel movement, pop-up mixers, and/or electro-magnets (the use of stationary electromagnet-based mixing of magnetic microparticles is described in detail in Example 2, below). In certain embodiments, positions W6-W8 will employ a heater, e.g., a resistive heater, to heat the lysis sample to about 40° C. In certain embodiments, W6-W7 employ incubation in Wash 1 for about 72 seconds. In certain embodiments, position W8 involves transfer of the microparticles into well P2 and mixing. In certain embodiments, Wash 2 is water. In certain embodiments, the transfer can be accomplished using moveable magnets above the wells to slide the microparticles across the internal surface of the seal to collect, transfer and release the microparticles. In some embodiments, transfer can be accomplished using moveable permanent magnets below the wells to slide the microparticles within internal channels at the bottom of wells to collect, transfer and release the microparticles. In certain embodiments stationary electro-magnets, e.g., selectively turning on/off adjacent magnets to achieve magnetic particle movement can be employed. Other methods of transferring microparticles such as in inverse particle processing can also be used. In certain embodiments, position W8 comprises mixing in the wash solution at well P2 for about 24 seconds. In certain embodiments, mixing is accomplished via carousel movement, a pop-up mixer, and/or electro-magnets.

Exemplary position W9 involves transfer of the microparticles into the wash solution at well P3 and mixing. In certain embodiments, the wash solution at well P3 is water. In certain embodiments, the transfer can be accomplished using moveable magnets above the wells to slide the microparticles across the internal surface of the seal to collect, transfer and release the microparticles. In certain embodiments, position W9 comprises mixing in the wash solution in Well P3 for about 24 seconds. In certain embodiments, mixing is accomplished via carousel movement, a pop-up mixer, and/or electro-magnets. In certain embodiments, mixing is accomplished using an offline orbital mixer at 1500 rpm. In certain embodiments stationary electro-magnets, e.g., selectively turning on/off adjacent magnets to achieve magnetic particle mixing can be employed.

In certain embodiments, positions W10-W17 correspond to an incubation position where the sample is incubated within the elution buffer to remove the target from the microparticles. Strategies for elution of nucleic acids from solid supports, e.g., microparticles are known in the art. For example, but not by way of limitation, nucleic acids, e.g., RNA and/or DNA, can be eluted by contacting the solid supports to which the nucleic acids are bound, e.g., microparticles, with an elution buffer with or without concurrent heating. In certain embodiments, the elution buffer comprises phosphate (e.g., inorganic phosphate or organophosphate) at a concentration of 1 to 10 mM (e.g., 1, 2, 3, 4, 5, 6, 7, 8, 9, or 10 mM). In some embodiments, the phosphate concentration is chosen to preferentially bind and/or elute the nucleic acid, e.g., DNA or RNA. In certain embodiments, the elution buffer comprises 5 mM PO₄.

Heating during positions W10-W17 incubation can be accomplished using, e.g., resistive convection heaters. For example, but not limitation, the resistive convection heater can are capable of heating the sample at these positions to about 80° C. In certain embodiments, the incubation extends for about 192 seconds. At the completion of the incubation, at position W18, the microparticles are transferred back into the wash solution of well P3, e.g., via moveable permanent magnets or the use of electro-magnets, leaving substantially microparticle-free eluate in well P4. For example, such transfer can occur at exemplary position W18, approximately 40 μl (+1-5%) of eluent is aspirated (e.g., after an about 12 second delay to cool eluent to 40° C.-60° C.), and half of the eluent is dispensed into one or more amplification vessel.

The below table, Table 7, depicts exemplary times and operations regarding the wash and elution process. As shown in this exemplary embodiment using a lockstep of 24 seconds, the sample processing time for the wash and elute process is 324 seconds, or 5.4 minutes.

TABLE 7

| | | Sample Process |
|---|---|---|
| Pos. | Function | Time (Seconds) |
| W1 | Load Wash Vessel in Carousel | |
| W2 | Dispense Partial Wash 1 | |
| | (250 μl +/− 5%) | |
| | Pickup Transfer Tip | |
| | Pierce Lysis Tube | |
| | Capture μParticles | |
| W3 | Transfer μParticles | |
| W4 | Dispense Remainder Wash 1 | |
| | (250 μl +/− 5%) | |
| | Dispense Wash 2 | |
| | (100-500 μl +/− 5%) | |
| | Dispense Wash 3 | |
| | (100-500 μl +/− 5%) | |
| | Dispense Elution Buffer) | |
| | (40 μl +/− 5%) | |
| W5 | Seal Wash Vessel | |
| W6-7 | Incubation (37° C.) | 72 |
| | Mixing Wash 1 (72 seconds) | |
| | 21 Mixes (wide field) | |
| W8 | Transfer μParticles to Wash 2 | 24 |
| | Mixing Wash 2 (24 seconds) | |
| | 12 Mixes (wide field) | |
| W9 | Transfer μParticles to Wash 3 | 24 |
| | Mixing Wash 2 (24 seconds) | |
| | 12 Mixes (wide field) | |
| W11 | Transfer μParticles to Elution | |
| W10- | Incubation (80° C.) | 192 |
| 17 | Mixing Elution (192 seconds) | |
| | 64 Mixes | |
| W18 | Pierce Elution Seal | 12 |
| | Aspirate Eluent (wait 12 seconds) | |
| | (42 μl +/− 5%) | |
| | Dispense Eluent to Amp Vessel 1 | |
| | (20 μl +/− 5%) | |
| | Dispense Eluent to Amp Vessel 2 | |
| | (20 μl +/− 5%) | |
| | Wash Probe | |
| | Indexing | |
| W19 | Dispense Master Mix and Activator | |
| W20 | Cap and Transfer Amplification Vessels | |
| W21 | Aspirate Wash Contents to Waste | |
| | Transfer Wash Vessel to Waste | |
| | Indexing | |

For purpose of example and not limitation, an additional exemplary wash and elution process is shown below in Table 7.1, including exemplary times and operations regarding the wash and elution process.

TABLE 7.1

| | | Sample Process |
|---|---|---|
| Pos. | Function | Time (Seconds) |
| W1 | Load Wash Vessel in Carousel | |
| W2 | Dispense Wash 1 (Lysis) (500 μl +/− 5%) | |
| | Dispense Wash 2 (Water) (250 μl +/− 5%) | |
| | Dispense Wash 3 (Water) (125 μl +/− 5%) | |
| W3 | Pickup Transfer Tip | |
| | Capture uParticles | |
| | Transfer uParticles | |
| W4-W7 | Incubation and Mixing (Wash 1) | |
| W8 | Transfer uParticles to Wash 2 | |
| W9 | Mixing (Wash 2) | |
| | Preheat Elution Well | |
| W10 | Transfer uParticles to Wash 3 | |
| | Preheat Elution Well | |
| W11 | Mixing (Wash 3) | |
| | Preheat Elution Well | |
| | Dispense Elution Buffer | |
| W12 | Transfer uParticles to Elution Well | |
| | Incubate Elution Well | |
| W13-W20 | Incubation and Mix Elution | |
| W21 | Transfer uParticles to Wash 3 | |
| | Cool Elution Well | |
| W22 | Aspirate Eluent | |
| | Dispense Eluent to Amp Vessel 1 | |
| | Dispense Eluent to Amp Vessel 2 | |
| W23 | Aspirate wash contents to waste | |
| W24 | Transfer Wash Vessel to waste | |

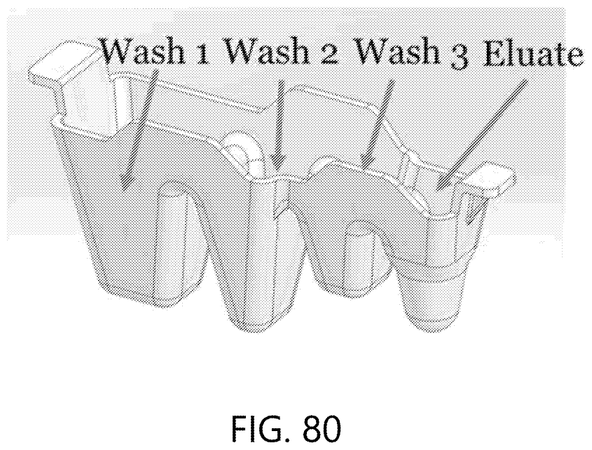

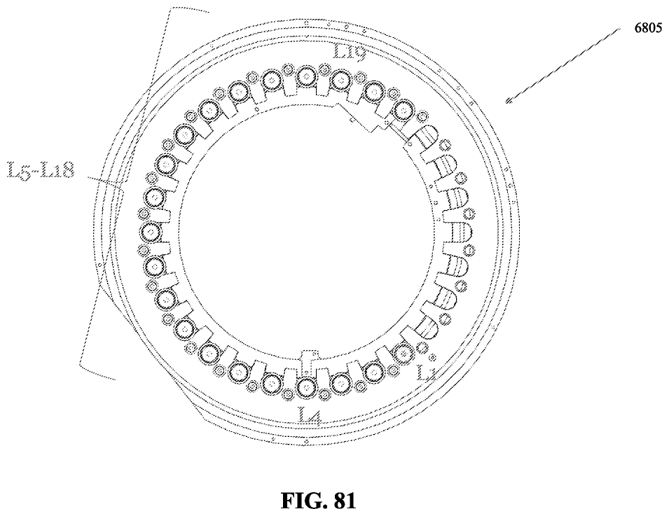

For example and as embodied herein, the wash and elution process can be performed in a wash vessel such as that depicted in FIG. 80, which can be moved around a wash track, such as that depicted in FIG. 70. Additionally or alternatively, uParticles can be transferred between wells of the wash vessel using, for example, permanent magnets, as described further herein.

For purpose of example and not limitation, the temperature of the wash vessel can be controlled. Additionally or alternatively, different areas of the was vessel can be controlled at different temperatures. For purpose of example and as embodied herein, Wash 1 and Wash 2 can have a temperature of approximately 40 degrees Celsius. Additionally or alternatively, and as further embodied herein, the Elution Well can be gradually heated to a desired temperature. For example, at wash track position W9 the contents of the Elution Well can be preheated to a temperature of between approximately 40° C. and approximately 70° C., at position W10 the contents of the Elution Well can be preheated to a temperature of between approximately 60° C. and approximately 70° C., at position W 11 the contents of the Elution Well can be preheated to a temperature of approximately 70° C., and at positions W12-W20 the contents of the Elution Well can be incubated at a temperature of approximately 80° C. As further embodied herein, the contents of the Elution Well can be cooled at position W21.

In certain embodiments more than three washes can be employed in the context of the instant disclosure. Moreover, the systems of the present disclosure a capable of processing distinct samples, e.g., serum/plasma samples and whole blood samples, including the with respect to the above-described steps or any step described below, such that individual samples can be handled in distinct manners in a single batch. For example, but not by way of limitation, individual samples in a batch can be incubated for longer or shorter periods and/or can be heated or cooled at distinct temperatures.

In certain embodiments, the plurality of well plates 10 identified in FIG. 7 can be arranged as illustrated in FIG. 17. For example, but not by way of limitation, positions P1, P2, P3, and P4 of FIG. 7 can correspond to Wash 1, Wash 2, Wash 3, and Eluate of FIG. 17. In certain embodiments where the plurality of well plates is arranged as illustrated in FIG. 17, one or more steps of the sample preparation process can be performed by a stationary electromagnet-based capture and movement of magnetic particles.

FIG. 17 shows a schematic of a wash vessel according to one embodiment of the present disclosure. A side view and a top view are shown. The wash vessel 1700 can include four wells, labeled as Wash 1-3 and Eluate, where the first well is adjacent a second well and a third well adjacent the second well, and a fourth well is adjacent the third well. A first side wall 1701 defines a first side of the first, second, third, and fourth wells. A second side wall 1702 opposite the first side wall defines a second side of the first, second, third, and fourth wells. The first and second side walls include an inner surface facing the first, second, third, and fourth wells and an outer surface opposite the inner surface. A first divider 1703 separates the first and second well and a second divider 1704 separates the second and third wells. The first side wall 1701 is higher than the first divider 1703 and the inner surface of the first side wall is substantially planar at least in a region extending between the first and second wells for translation of magnetic microparticles captured in the first well over the first divider into the second well. The inner surface of the first side wall 1701 is non-planar in a region extending between the second and third wells such that the non-planar inner surface prevents translation of magnetic particles captured at a region of the inner surface in the second well to a region of the inner surface in the third well. The second side wall 1702 is higher than the second divider 1704. The inner surface of the second side wall 1702 is substantially planar at least in a region extending between the second and third wells and provides a substantially planar surface for capture of magnetic microparticles in the second well and translation of the captured magnetic particles over the second divider 1704 to the third well. The first and second wells can include a wash solution and the third well can include a wash solution or an elution solution. A third divider 1705 separates the third well from the fourth well. The first side wall is higher than the third divider 1705 and the inner surface of the first side wall is substantially planar at least in a region extending between the third and fourth wells for translation of magnetic microparticles captured in the third well over the third divider 1705 into the fourth well, and the inner surface of the second side wall 1702 is non-planar in a region extending between the third and fourth wells such that the non-planar inner surface prevents translation of magnetic particles captured at a region of the inner surface of the second side wall in the third well to a region of the inner surface of the second side wall in the fourth well. The first, second, and third wells may include a wash solution and the fourth well may include an elution solution. The non-planar inner surface may be rendered non-planar by presence of a notch 1706, 1707 at the inner surface, where the notch extends out of the inner surface or extends into the inner surface.

Operational steps involving a stationary electromagnet-based capture of magnetic particles include but are not limited to: mixing; washing; and transfer of the magnetic particles. Existing methods of mixing and washing magnetic particles generally rely on mechanical agitation and magnetic mixing using moving permanent magnets. In certain embodiments, however, such mixing, washing, and/or transfer can be performed with a system incorporating at least one stationary electromagnet-based capture of magnetic particles, e.g., as exemplified in FIGS. 42A-42D. For example, but not limitation, and as embodied in FIG. 42A, magnetic microparticles 4202 can be present in a first vessel of a plurality of vessels 4206. Alternatively, or additionally, an electromagnet 4204 can be used to capture the magnetic microparticles to the wall of the vessel 4206, as illustrated in FIG. 42B. Alternatively, or additionally, movement of the electromagnet 4204 to a second vessel induces the transfer of the magnetic microparticles 4202 from the first vessel to the second vessel, as illustrated in FIG. 42C. Alternatively, or additionally, elimination of the current to the electromagnet 4204 once the magnetic microparticles 4202 have transferred to the second vessel allows for release of the magnetic microparticles 4202 as illustrated in FIG. 42D. Alternatively, or additionally, one or more of the distinct mixing or washing positions identified herein can be accomplished using a stationary electromagnet-based capture of magnetic particles. In addition, certain embodiments described herein can be performed with a system incorporating at least one stationary electromagnet-based capture of magnetic particles in connection with one or more transfer operations at one or more of the transfer positions within the systems described herein. As will be appreciated by those of skill in the art, the specific number, orientation, and operational assignment (e.g., mixing, washing, transfer, or elution) of the individual positions can be modified as desired and yet remain within the scope of the present disclosure.

In certain embodiments, a method of washing microparticles for nucleic acid analysis may involve providing a wash vessel comprising a first well, a second well adjacent the first well, a third well adjacent the second well, and a fourth well adjacent the third well as depicted in FIGS. 42A-42D. A first side wall 4201 defines a first side of the first, second, third wells, and fourth wells. A second side wall 4203 opposite the first side wall 4201, defines a second side of the first, second, third and fourth wells. The first and second side walls include an inner surface facing the first, second, third and fourth wells and an outer surface opposite the inner surface. A first divider 4206 separates the first and second well, the first side wall is higher than the first divider and the inner surface of the first side wall is substantially planar at least in a region extending between the first and second wells. A second divider 4207 separates the second and third wells and the inner surface of the second side wall is substantially planar at least in a region extending between the second and third wells. The method may include introducing the magnetic microparticles into the first well, where the first well includes a wash solution, applying a magnetic force to capture the magnetic microparticles 4202 on an inner surface of the first side wall in the first well; translating the captured magnetic microparticles from the first well over the first divider to the second well along the inner planar surface of the first side wall, where the second well contains a wash solution; removing the magnetic force and allowing the magnetic microparticles to be released into the second well from the inner surface; applying a magnetic force to capture the magnetic microparticles on the inner surface of the second side wall opposite the first side wall, where the second side wall is higher than the second divider, translating the captured magnetic microparticles from the second well over the second divider to the third well along the inner planar surface of the second side wall, where the third well contains a wash solution or an elution solution; and removing the magnetic force and allowing the magnetic microparticles to be released into the third well from the inner surface of the second side wall. In certain embodiments, the third well may contain a wash solution and the method may further include, applying a magnetic force to the magnetic microparticles in the third well to capture the magnetic microparticles on an inner surface of the first side wall and removing the elution solution. In another embodiments, the third well contains a wash solution and the fourth well contains an elution solution. The inner surface of the first side wall may be non-planar in a region extending between the second and third wells, and planar in a region extending between the third and the fourth wells. The inner surface of the second side wall may be non-planar in a region extending between the third and fourth wells. The planar regions are conducive to translation of captured magnetic microparticles across the inner surface and the non-planar regions do not allow translation of captured magnetic microparticles across the inner surface. The method may further include applying a magnetic force to capture the magnetic particles in the third well on an inner surface of the first side wall, where the first side wall is higher than a third divider 4208 separating the third well from the fourth well; translating the captured magnetic microparticles from the third well over the third divider to the fourth well along the inner surface of the first side wall; and removing the magnetic force and allowing the magnetic microparticles to be released into the fourth well from the inner surface of the first side wall. The method may further include applying a magnetic force to the magnetic microparticles in the fourth well to capture the magnetic microparticles on an inner surface of the second side wall and removing the elution solution. The non-planar inner surface may include a notch 4209, 4210 at the inner surface. The notch may extend out of the inner surface or extends into the inner surface

The present disclosure contemplates the use, in certain embodiments, of stationary electromagnets in a variety of positions to accomplish the appropriate mixing, washing, and transfer operations. For example, but not by way of limitation, electromagnets can be positioned at right side, left side, and/or bottom side of a well. In addition, electromagnets can be positioned with one side higher than the other. The stationary electromagnets of the present disclosure can also be used in conjunction with a variety of well formats known in the art.

In certain embodiments, opposing electromagnets can be arranged to alternately attract magnetic particles to the opposite sides of a wash well. However, the position, timing, power, and sequence of the electromagnet activations is entirely flexible. For example, but not by way of limitation, stationary electromagnets and be used to partially collect magnetic particles and then allow them to drop to the bottom of the well. Alternatively, or additionally, magnetic particles can be completely collected alternately on the sides of a well to facilitate mixing, washing, and/or transfer. In addition, magnetic particles can be slowly collected with lower power or magnetic particles can be more rapidly collected with higher power.

Distinct types of electromagnets are also contemplated for use in connection with the embodiments disclosed herein. For example, while DC electromagnets can be used, because DC electromagnets can magnetize magnetic particles, AC electromagnets can also be used in conjunction with or in place of DC electromagnets to avoid creating residual magnetism of the magnetic particles by varying the switching frequency.

The use of electromagnets in addition to or in lieu of permanent magnets (or other mixing, washing, or transfer strategies) provides several advantages. Mixing or transfer operations during sample preparation typically requires permanent magnets to be moved in and out of range of the magnetic particles. The use of electromagnets can eliminate motion mechanisms by simply turning on and off the electromagnets. In addition, magnetic particles can be moved from one well to another by successively turning on and off adjacent magnets within an array. Incorporating a stationary electromagnet-based particle capture approach can also eliminate one or more disposable per test, thus reducing the amount of solid waste being generated. The use of stationary electromagnets also eliminates the need for specific volume requirements and disposable coverings for the wells or moving permanent magnet when transferring magnetic particles from one well to another. The instant strategy can also transfer magnetic particles between wells using moving electromagnets adjacent to the side of the wash vessel, but not touching the magnetic particles or liquid within wells, minimizing magnetic particle loss and liquid carryover from source to destination wells, thus maximizing assay performance.

In certain embodiments, transfer can be accomplished using moveable permanent magnets below the wells to slide the microparticles internal channels at the bottom of wells to collect, transfer and release the microparticles. In certain embodiments stationary electro-magnets, e.g., selectively turning on/off adjacent magnets to achieve magnetic particle movement can be employed. Other methods of transferring microparticles such as in inverse particle processing can also be used.

### 6.1.3 Eluate Transfer Systems

After the target nucleic acid has been eluted from the microparticles, the resulting eluate can be separated from the microparticles by, for example, pipetting the eluate from the elution well and aspirating it to another vessel for amplification or removing the microparticles from the elution well. In certain embodiments, some or all of the eluate is transferred to a single reaction vessel which then undergoes an amplification process.

In certain embodiments, the eluate is split into a plurality of amplification vessels. In certain embodiments, a first portion of the eluate can be transferred to a first amplification vessel and a second portion of the eluent can be dispensed into a second amplification vessel. An exemplary embodiment of transferring and splitting eluate into an amplification process according to the exemplary embodiment shown in FIG. 64 and FIG. 65 and is described below with respect to FIG. 67A and FIG. 67B.

In certain embodiments, a “split eluate” approach is applicable to any elution strategy described herein. For example, and as illustrated in FIG. 67A and FIG. 67B, in addition to reducing the number sample preparations performed (and the associated consumables), splitting of the eluate can be used to increase the number of nucleic acid analyses performed, e.g., by creating additional opportunities for multiplexed amplification reactions from a single sample aspiration.

For example, in certain embodiments, the systems described herein are capable of aspirating a single sample for sample preparation every 24 seconds, which results in 150 sample aspirations per hour. In certain embodiments, and as illustrated in the top process of FIG. 67A, each sample aspirated is prepared and a single target nucleic acid is amplified and detected, leading to 150 results. In certain embodiments, for example as embodied in the bottom process of FIG. 67A, each aspirated sample is prepared and the eluate containing nucleic acids for amplification is split into two amplification vessels, where each amplification reaction amplifies a single target nucleic acid for detection, leading to 300 results. In certain embodiments, e.g., Scenarios 2 and 4 of FIG. 67B, each aspirated sample is prepared, and the eluate split into two amplifications, where each amplification reaction amplifies two target nucleic acids for detection, leading to 600 results. In certain embodiments, each aspirated sample is prepared and the eluate split into two amplifications, where each amplification reaction amplifies four or more target nucleic acids for detection, leading to 1,200 or more results.

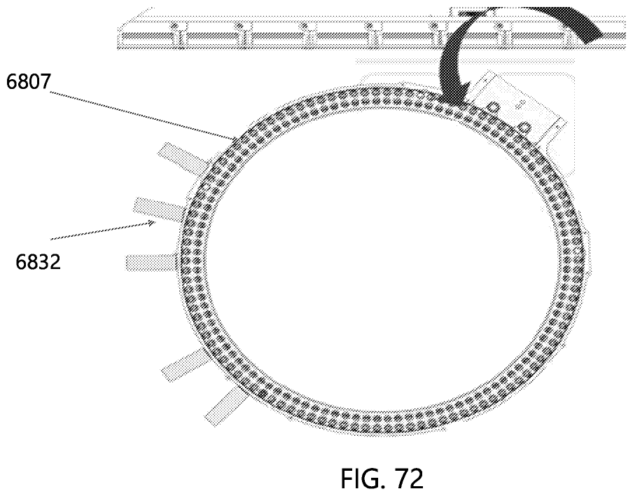

FIG. 72 depicts an exemplary amplification and detection system 6807 for use with the disclosed subject matter described herein. As embodied herein, the amplification and detection system 6807 can be in a round shape, rotating in a counterclockwise or clockwise direction. As embodied herein, the amplification and detection system 6807 can include 107 positions about the circumference of the amplification and detection system 6807. The shape, size and configuration of the amplification and detection system can be selected based on the desired performance of the system. As further embodied herein, each position on the amplification and detection system 6807 can include two openings for receiving two amplification vessels. For purpose of illustration not limitation, the amplification and detection system 6807 includes two separate reagents in two amplification vessels. As embodied herein, each reagent can be mixed in the amplification vessels in about 24 seconds. For purpose of illustration not limitation, an exemplary pipettor can aspirate and dispense samples into the pre-loaded amplification vessels. As embodied herein, the amplification vessels located on the amplification and detection system 6807 can assay specific RPA reaction components, reagents, and/or enzymes. As embodied herein, the amplification and detection system 6807 rotates and advances one position in about every 12 seconds. For purpose of illustration not limitation, the amplification and detection system 6807 can include five fluorescent readers 6832 at different wavelengths to conduct optical detection of the amplification vessels. For illustration and not limitation, and as embodied herein, amplification and detection system 6807 can be integrated with other components of sample analysis systems as described further herein. For example, as embodied herein and with reference to FIGS. 68A-68D, eluate can be introduced to amplification and detection system 6807 using eluate pipettor 6813, which can be a robotic pipettor as described further herein. As embodied herein, eluate pipettor 6813 can transfer eluate from a wash vessel, such as for example wash vessel 4200, which can be carried by a wash track, such as for example wash track 8002.

In certain embodiments, splitting an eluate as described herein can reduce assay development complexity and schedule. In addition, splitting an eluate as described herein can double the number of nucleic acid analyses performed for one sample preparation. For example, but not by way of limitation, assays requested contemporaneously can be grouped together to take advantage of eluate splitting; such as a “Mosquito Panel” of Chikungunya and Dengue or West Nile and Zika. Alternatively, or additionally, a single sample aspiration can be prepared by the methods and systems described herein and the nucleic acid eluate so prepared can be split into two amplifications reactions where one amplification reaction includes reagents suitable to amplify HIV-1, HIV-2, HCV, and HBV (e.g., an HxV multiplex amplification) and the second amplification reaction includes reagents suitable to amplify Parvovirus and HAV. In certain embodiments, the ability to detect HIV-1, HIV-2, HCV, HBV, Parvovirus and HAV from a single sample aspiration find particular use in connection with screening of plasma samples. As a result of performing less sample preparations per assay, splitting an eluate as described herein can reduce the solid waste associated with the eliminated sample preparations. Furthermore, as a result of performing less sample preparations per assay, splitting an eluate as described herein reduces the liquid and solid waste associated with the eliminated sample preparations. For example, but not by way of limitation, splitting an eluate can reduce reagents, buffers and/or microparticles used to produce a given number of results. Additionally or alternatively, splitting eluate can reduce the number of lysis tubes and or lysis tips used to produce a given number of results. Additionally or alternatively, splitting eluate can reduce the number of wash vessels used to produce a given number of results.

### 6.1.5 Additional Sample Preparation System Embodiments

Alternatively, or in addition to the systems described above, the present disclosure encompasses sample preparation systems comprising one or more of the following processes and/or system components.

### 6.1.5(a) Stationary Sample Preparation

As embodied herein, for illustration and not limitation, and in accordance with another aspect of the disclosed subject matter, the automated analysis systems of the present disclosure, e.g., as illustrated in FIG. 54B, include a sample preparation station 3110. The sample preparation station can include one or more (e.g., 2, 3, 4, or more) sample preparation positions. In certain embodiments, sample preparation vessels can be transported to the one or more sample preparation positions by a robotic handler. In certain embodiments, the vessels transported to the sample preparation positions include samples, but also can include some or all of the reagents necessary for the sample preparation process.

Additionally, or alternatively, the sample lysis, wash, and elution can be performed in a single, stationary sample preparation vessel. For example, but not limitation, a sample can be aspirated from a sample tube 3102 and dispensed into a sample preparation vessel where it is contacted by a sample lysis buffer and magnetic microparticles, dispensed by, e.g., a moveable robotic pipettor 3114. Upon binding of nucleic acids to the magnetic microparticles, a movable permanent magnet or a stationary electro-magnet can be used to retain the magnetic microparticles within the sample preparation vessel while the sample lysis buffer is aspirated and a wash buffer is dispensed into the sample preparation vessel. The aspiration of the sample lysis buffer and dispensing of wash buffer can be performed by a moveable robotic pipettor 3114 that can traverse a plurality of positions, e.g., to aspiration reagents from reagent supply container, dispense reagents into the sample preparation vessel, and dispense waste into a liquid waste container. The nucleic acid bound magnetic microparticles can then be washed by mechanical or magnet driven movement of the microparticles. At the completion the first wash, the nucleic acid bound magnetic microparticles can again be retained within the sample preparation vessel by contacting the vessel with a moveable permanent magnet or a stationary electro-magnet while the wash fluid is aspirated and a second wash fluid is dispensed into the sample preparation vessel. Multiple washes can be performed in this fashion. After the final wash, the magnetic microparticles can be retained in the sample preparation vessel using a moveable permanent magnet or a stationary electro-magnet while the wash fluid is aspirated, and an elution buffer is dispensed into the sample preparation vessel. At the completion of the elution, the magnetic microparticles can again be retained within the sample preparation vessel with a moveable permanent magnet or a stationary electro-magnet while the eluate is aspirated and dispensed, e.g., in one or more amplification vessels for amplification of the eluted nucleic acid. Non-limiting examples of such amplification systems are described in detail in EP1623764 and US2019/0284612, the entire contents of which are incorporated by reference herein.

Additionally, or alternatively, such sample preparation systems comprise a processing deck upon which sample preparation vessels can be located, e.g., a sample preparation station 3110. In certain embodiments, the processing deck will be surrounded by a housing that allows a plurality of assembly rings, a support structure cantilevered over, or a support structure fixed over the processing deck, e.g., as illustrated in FIG. 54B. In certain embodiments, the housing or support structure can be configured to position one or more robotic pipettors capable 3114 of introducing or aspirating fluid into the sample preparation vessels located on the processing deck. In certain embodiments, addition assembly rings or support structures can be configured to hold sample preparation reagents and/or disposal receptacles. In certain embodiments, the processing deck will include a plurality of heating and/or cooling units configured to facilitate the heating and cooling of the sample preparation vessels. Additionally, or alternatively, such heating units can be an oven in which sample preparation reactions occur. In certain embodiments, the processing deck can comprise a mixing unit configured to facilitate mixing of the sample preparation vessels. Additionally, or alternatively, the sample preparation system can include incubators, e.g., rotary incubators, configured to maintain the sample preparation vessels at a predetermined temperature for a predetermined length of time.

### 6.1.5(b) Sample Preparation in Purpose-Shaped Tip

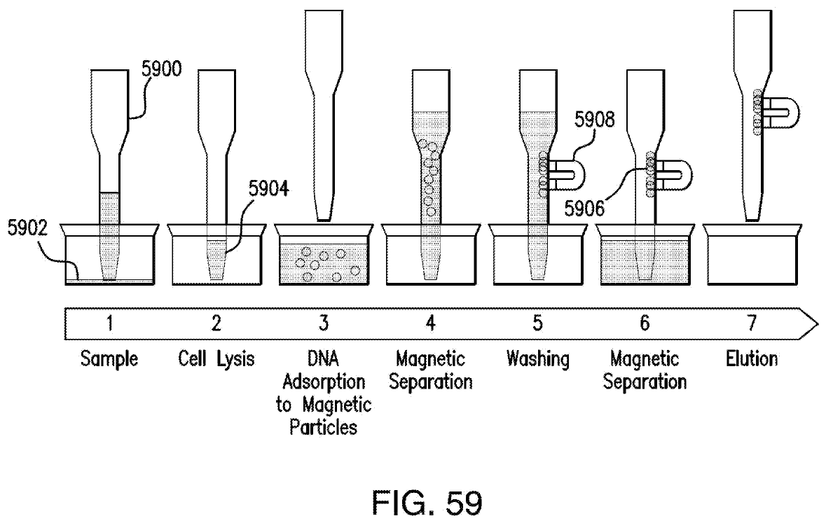

In certain embodiments, e.g., as exemplified in FIG. 59, sample preparation is performed using purpose-shaped plastic disposable tips 5900 configured to interact with a magnet 5908 external to the tip. For example, but not limitation, such purpose-shaped tips 5900 can be used to aspirate a sample 5902. The sample can then be expelled from the tip into a sample lysis buffer, e.g., any suitable sample lysis buffer as embodied herein. In certain embodiments, the sample lysis buffer will comprise magnetic microparticles 5906, while in certain embodiments, such magnetic microparticles 5906 can be introduced after the sample is contacted with the sample lysis buffer, e.g., as illustrated in FIG. 59. Introduction of the magnetic microparticles, e.g., CuTi microparticles for total nucleic acid capture or microparticles comprising immobilized oligonucleotides to facilitate target nucleic acid capture, allows for capture of sample nucleic acid for further processing, e.g., as illustrated in FIG. 59. For example, but not limitation, the purpose-shaped tips 5900 allow for the aspiration of magnetic microparticles 5906 suspended in fluid, e.g., cell lysate, wash buffer, or elution buffer, and retention of the particles via the application of magnetic force by the external magnet 5908 and dispensing of the fluid from the tip and aspiration of anew (identical or different) fluid. Upon successive washes mediated by repeated aspiration and dispensing of wash fluid while the magnetic microparticles 5906 are retained within the purpose-shaped tip 5900, e.g., as illustrated in FIG. 59, the nucleic acids bound to the magnetic microparticles 5906 can be eluted by aspiration of an elution fluid to contact the magnetic microparticles 5906 retained within the purpose-shaped tip 5900 as illustrated in FIG. 59. Dispensing the elution fluid after contact with the magnetic microparticles 5906 into a suitable vessel, e.g., a well or tube, can be accomplished by dispensing the elution fluid from the purpose-shaped tip 5900 while retaining the magnetic microparticles 5906 within the purpose-shaped tip 5900, as illustrated in FIG. 59.

In certain embodiments, release of the magnetic particles from the interior of the purpose-shaped tip 5900 can be achieved by the timed removal of the magnetic force. In certain embodiments, the technology is referred to as Magtration® Technology (Precision System Science Co.) (FIG. 59). Exemplary embodiments of such sample preparation strategies can be found in, e.g., U.S. Pat. Nos. 7,157,047 and 8,142,737, each of which is incorporated herein by reference in its entirety.

### 6.1.5(c) Inverse Magnetic Particle Processing

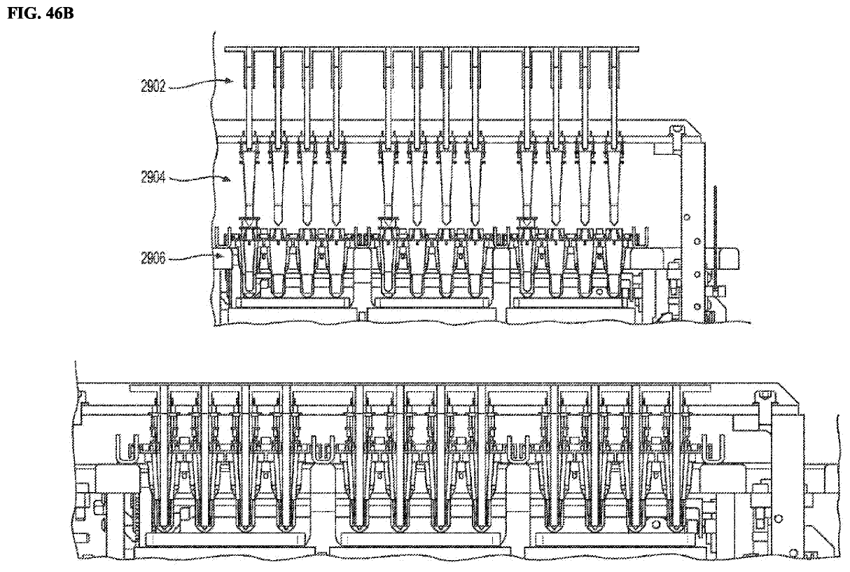

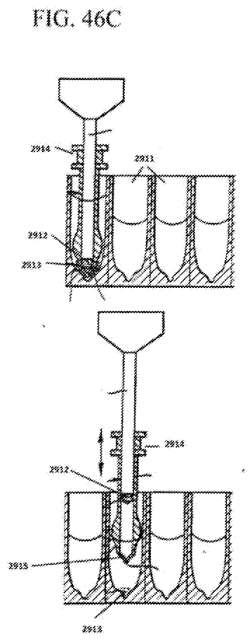

In certain embodiments, the operating principle employed by the sample preparation systems of the present disclosure is inverse magnetic particle processing technology. Rather than moving liquids while retaining magnetic particles in a sample preparation vessel, e.g., via magnets as described in Section 6.1.5(a) above, the magnetic particles, themselves, are moved in the inverse magnetic particle processing approach. In certain embodiments, e.g., as illustrated in FIG. 46C, the inverse particle processing involves moving magnetic microparticles from one well in a container holding wash related solutions to another well 2911 by inserting a magnet 2912 into the plunger 2914 which attracts magnetic particles 2913 to the external wall 2915 of the plunger 2914. Once the particles 2913 are magnetically attached to the plunger 2914, it is moved to the next well 2911. The particles 2913 are released by removing the magnet 2912 from inside the plunger 2914.

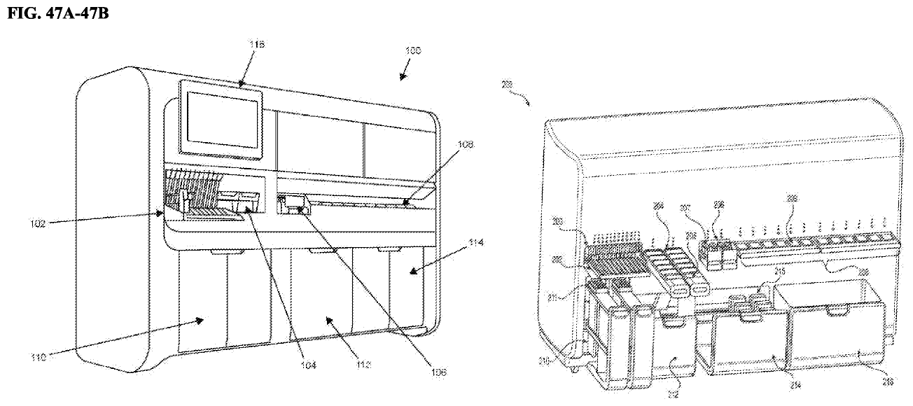

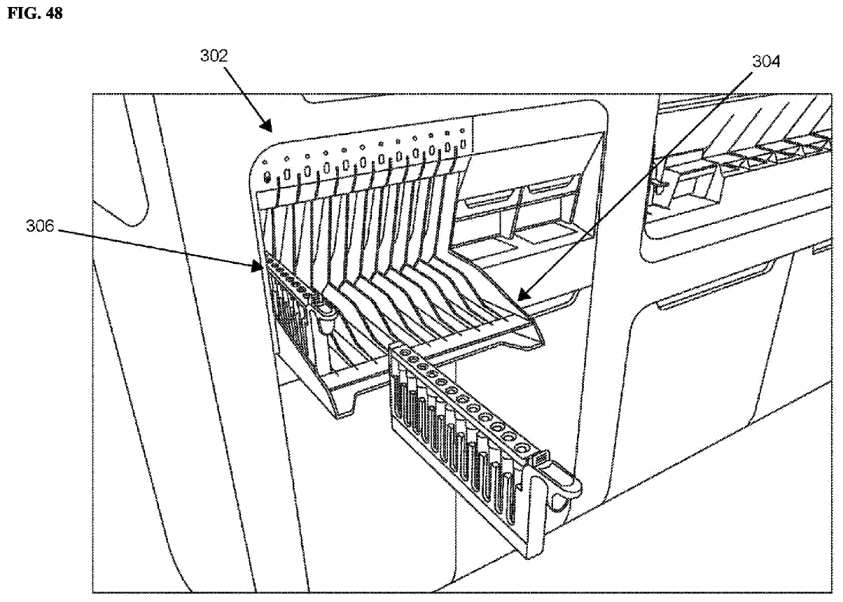

For purpose of illustration and not limitation, an external view of an automated analysis system according to one embodiment of the present disclosure is shown in FIG. 47A. In this example, system 100 is an automated nucleic acid preparation and analysis system that includes sample loading area 102, pipette tip loading area 104, ancillary reagent loading area 106, assay reagent plate loading area 108, sample processing (SP) cartridge loading area 110, bulk fluid storage area 112 and solid waste storage area 114. Ancillary reagents can include magnetic particles, elution buffer, and/or the like that find use, e.g., in isolating, purifying and eluting nucleic acids at a sample preparation station of the system. The system includes local user interface (LUI) 116. LUI 116 includes a touchscreen display for displaying a graphical user interface to the operator. The display can enable the operator to locally view patient results, assess the status of the instrument, etc. Not shown in FIG. 47A are internal components of the system including a sample filling station, a sample preparation station, a sample analysis station, and other internal system components.

Further for illustration and not limitation is an external view of another automated analysis system according to an embodiment of the present disclosure is shown in FIG. 47B. In this example, system 200 is an automated nucleic acid preparation and analysis system that includes sample loading area 202, pipette tip loading area 204, ancillary reagent loading area 206, assay reagent plate loading area 208, sample processing (SP) cartridge loading area 210, reaction vessel waste storage area 212, bulk fluid storage area 214 and solid waste storage area 216. As shown in FIG. 47B, each loading or storage area includes a shelf or drawer to facilitate loading and/or removal of the relevant item(s) by an operator of the system, where the shelves or drawers of system 200 are shown in the open position in FIG. 47B. In this example, samples are present in tubes and loaded into the system in sample tube racks, including sample tube rack 203. Pipette tips 205 for use by a robotic pipettor of the system are shown in pipette tip loading area 204. Ancillary reagent packs, including ancillary reagent pack 207, are shown in ancillary reagent loading area 206. Assay reagent plates, including assay reagent plate 209, are shown in assay reagent plate loading area 208. SP cartridges, including SP cartridge 211, are shown in SP cartridge loading area 210. Bulk fluid (bulk reagent) containers, including bulk fluid container 215, are shown in bulk fluid storage area 214. Not shown in FIG. 47B are internal components of the system including a sample filling station, a sample preparation station, a sample analysis station, and other internal system components.

According to certain embodiments, samples loaded into the system are present in sample tubes. Sample tubes can be loaded into the system individually, or can be loaded together with other sample tubes within a sample tube rack 306, e.g., as illustrated in FIG. 48. When sample tube racks 306 are employed, the two or more sample loading positions can be two or more lanes into which the racks are loaded. For example, the sample tube loading area 302 can include a platform 304 having two or more lanes into which a user places sample tube racks 306. The lanes can be separated by lane dividers. The number of lanes can vary. In certain aspects, the loading area 302 includes from 2 to 20 lanes, such as from 5 to 15 lanes (e.g., 12 lanes).

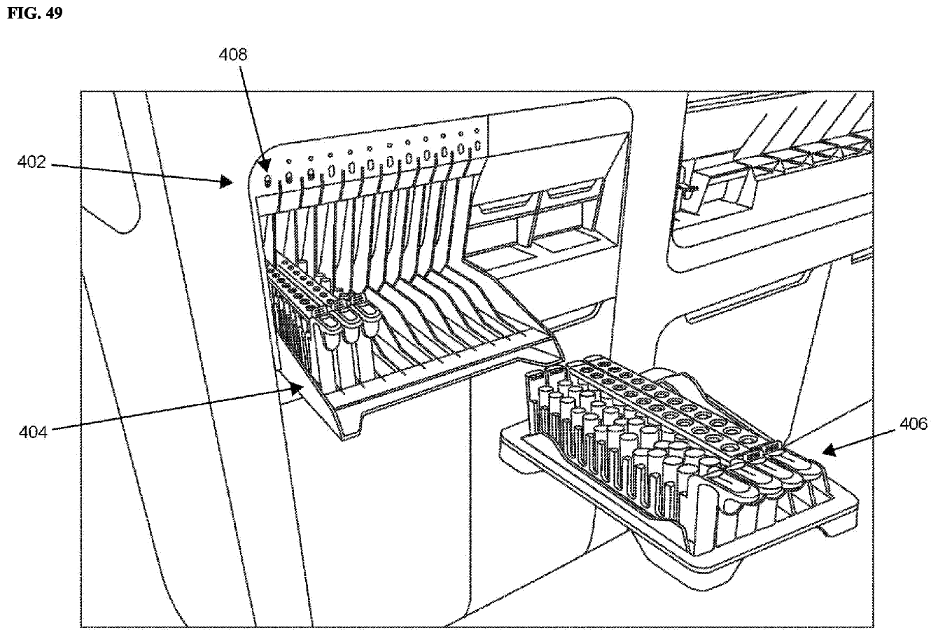

According to certain embodiments, e.g., as illustrated in FIG. 49, the sample loading area 402 includes a loading shelf 404 that pivots between a closed/up (unavailable) and open/down (available) loading position. In certain embodiments, the system can include a detached sample rack shelf 406 which can be loaded by an operator away from the system as the samples are available for preparation. Once the desired number of racks are loaded on shelf 404, the racks can be loaded from the shelf to the sample loading area 402 of the system, e.g., in a single motion. This functionality finds use, e.g., for consolidating high volume and low volume samples on one platform.

According to certain embodiments, e.g., as illustrated in FIG. 49, the sample loading area 402 includes lane indicator lights 408 aligned with each lane to indicate to the operator information including lane/processing status, lane availability, and/or the like.

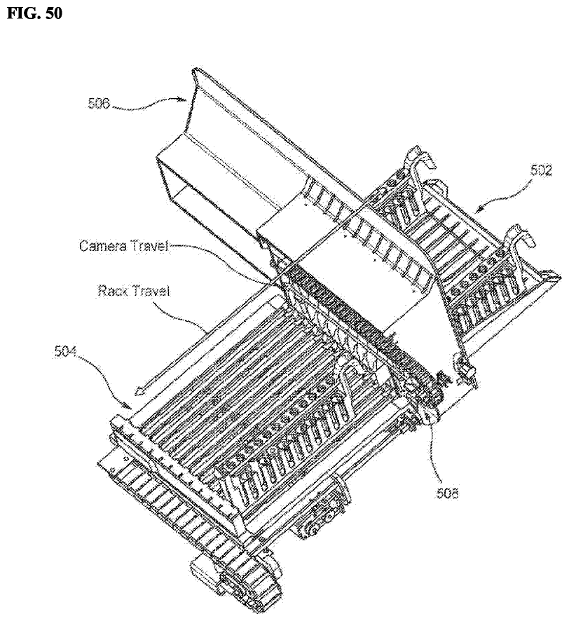

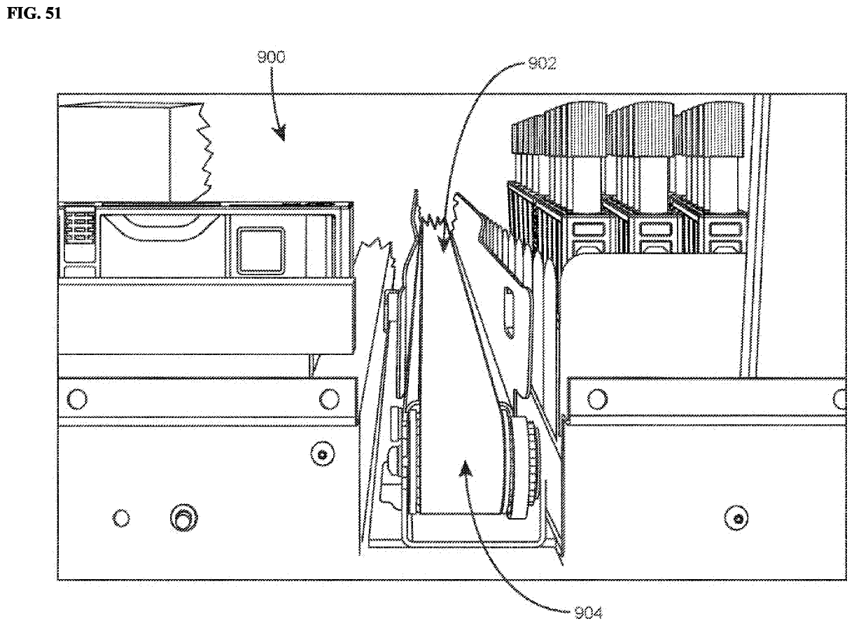

In certain aspects, e.g., as illustrated in FIG. 50, the sample loading area includes a sample tube identification code reader 508. The sample tube identification code reader 508 can be a sample tube barcode reader. The identification code can vary and in certain aspects is either a one-dimensional code, a two-dimensional code (e.g., a QR code), or the like. Upon determining that a particular sample tube rack will be transported from the sample rack shelf 502 to the internal sample rack area 504, the system moves the sample tube identification code reader 508 to a position adjacent to that of sample tube rack travel, and as the sample tube rack travels from the sample rack shelf 502 to the internal sample rack area 504, the barcode reader successively reads barcodes present on sample tubes present in the sample tube rack. The reader can also identify unique rack and tube characteristics that can be used by the system to prevent potential user errors.

As such, according to certain embodiments, the methods of the present disclosure can include reading sample identification information (e.g., a sample barcode) present on sample racks and sample tubes as sample tube racks are loaded into the system.

In certain aspects, the sample loading area includes a sample tube rack identification code reader. For example, e.g., as illustrated in FIG. 50, the sample loading area can include one or more cameras 508 disposed proximal to the sample tube rack area of the loading area for reading a barcode present on the racks.

According to certain embodiments, e.g., as illustrated in FIG. 50, the sample loading area includes a detection system, e.g., a camera, for detecting the presence and/or position of a rack within lanes (e.g., in the sample rack shelf 502 and/or in the internal sample rack area 504) of the sample loading area. The detection system can include position sensors provided in the front and/or rear of the sample loading area for detecting whether a rack is partially inserted, fully inserted, or has been ejected by the system upon completion of sample analysis. According to certain embodiments, when a rack is fully inserted into a lane, the rack is fixed into place by a fixing mechanism. For example, the rack can be fixed into place by a locking rib present on the surface of a lane that mates with a notch present on the underside of the rack. Reversible fixing of the rack upon full insertion deters the user from removing an in-process rack, while still allowing removal in case of, e.g., loss of power to the system.

In certain aspects, the sample loading area of the system is adapted to receive samples automatically from a different automated system, e.g., using the conveyor system 902 illustrated in FIG. 51. For example, the system 900 can function as a separate automated sample preparation and analysis system, or be integrated (e.g., configured in a workcell) with one or more other systems, e.g., one or more other automated sample preparation and analysis systems. A workcell can be adapted for automated transport of sample tubes (e.g., present in sample tube racks) between individual systems of the workcell. For example, a rack that includes one or more sample tubes present in a first system can be transported to a different system of the workcell, e.g., to balance the workload including the selection of specific nucleic acid analyses between the systems. For sample tube transport between systems of the workcell, e.g., as illustrated in FIG. 51, each system 900 can include an internal sample tube (or sample tube rack) conveyor system 902 that includes a conveyor belt 904. The systems can be positioned such that the conveyor belts 904 of the internal conveyor systems 902 of adjacent systems are aligned, permitting the transport of tube racks between systems. Details regarding internal sample tube (or sample tube rack) conveyor systems 902 that find use in transporting containers between systems/modules are described in U.S. Patent Application Ser. No. 62/269,535, the disclosure of which is incorporated herein by reference in its entirety.

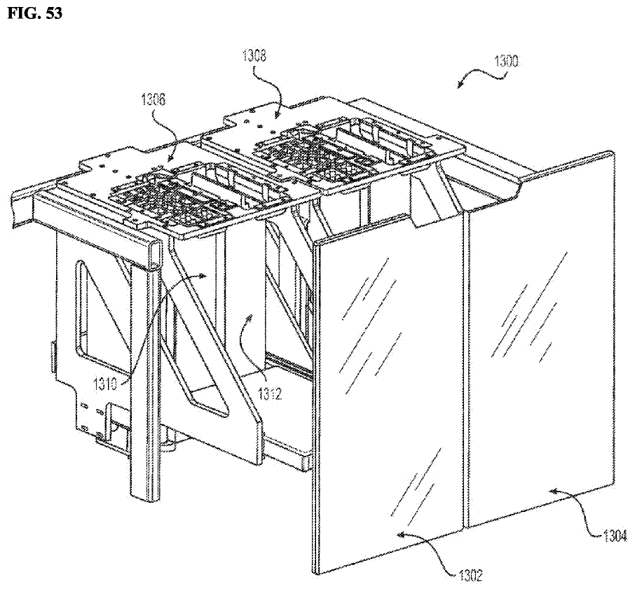

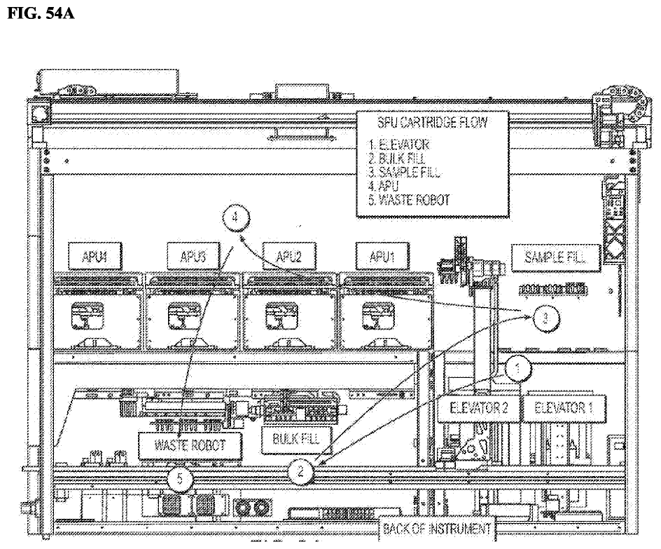

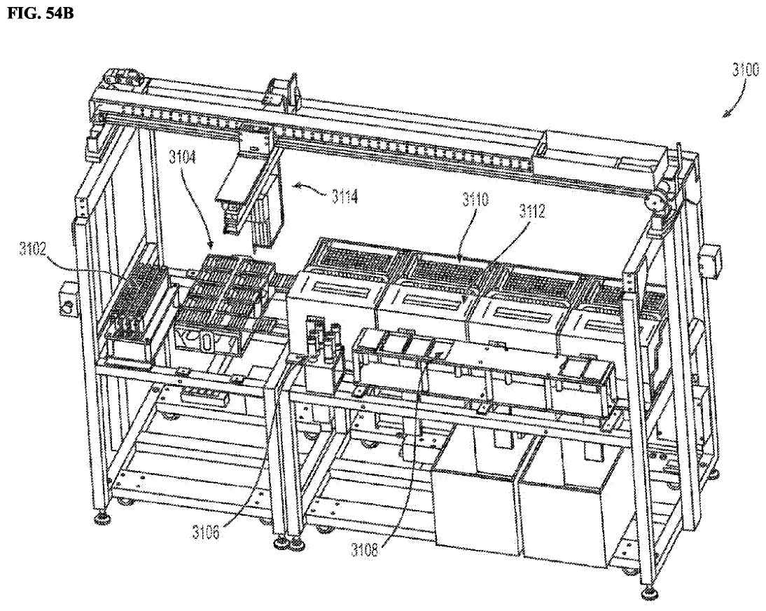

An automated sample analysis system of the present disclosure can employ sample preparation (SP) cartridges to facilitate sample preparation and, accordingly, can include an SP cartridge loading area 1300, e.g., as illustrated in FIG. 53. An exemplary pierceable septum for use with sample preparation cartridges is disclosed in U.S. Pat. No. 10,456,786 and is herein incorporated by reference in its entirety. An exemplary SP cartridge carrier design is disclosed in U.S. Design No. 937,435 and is herein incorporated by reference in its entirety. An exemplary sample deck design is disclosed in U.S. Design No. 799,999 and is herein incorporated by reference in its entirety. An exemplary sample tray design is disclosed in U.S. Design No. 791,965 and is herein incorporated by reference in its entirety.

An SP cartridge loading area 1300 can include doors 1302 and 1304 that provide the operator access to the internal portion of the loading area. In certain examples, the internal portion includes SP cartridge storage units 1306 and 1308 behind the doors. In certain aspects, each storage unit includes one or more SP cartridge elevators (see elevators 1310 and 1312 of storage unit 1306). The elevators are adapted to release SP cartridges at the top of the stack, one SP cartridge at a time, utilizing an upward force on the bottom of the stack. In certain aspects, the operator removes a sleeve pull tab to expose the SP cartridge stack to the elevator. The number of SP cartridges in a stack/sleeve can vary. In certain aspects, each stack/sleeve has from 10 to 30 SP cartridges, e.g., 20 SP cartridges.

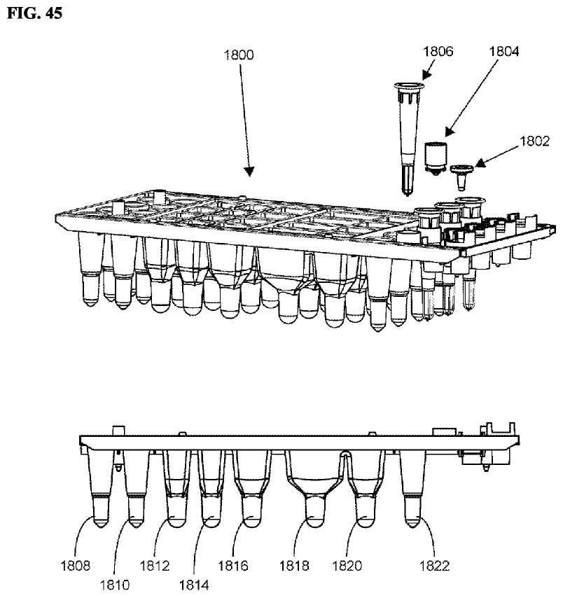

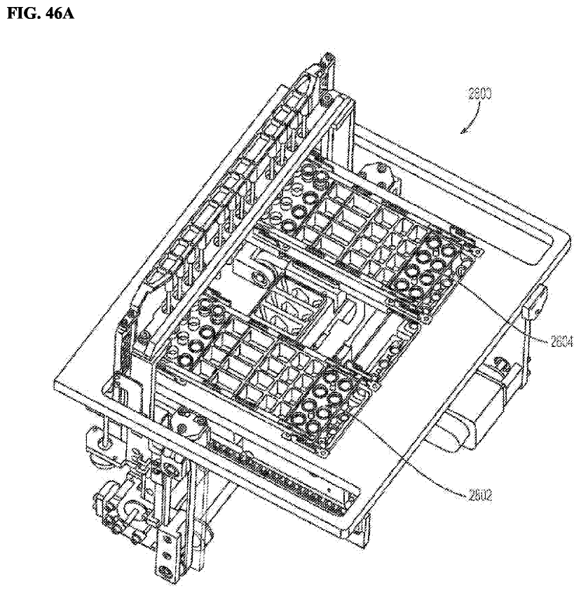

In certain examples, e.g., a sample preparation cartridge as illustrated in FIG. 45, SP cartridges 1800 can include removable reaction vessels (RVs) 1802, removable RV caps 1804, and removable plungers 1806. In certain aspects, the RV 1802 and RV cap 1804 are as described in U.S. Patent Application Publication No. 2017/0268039, the disclosure of which is incorporated herein by reference in its entirety. As is described in greater detail herein, the plunger 1806 finds use, e.g., in embodiments in which sample preparation involves magnetic particle-based capture of nucleic acids. In certain embodiment, the sample preparation systems described herein will comprise a plurality of wells 1808-1822. For example the well can be an elution well 1808, an auxiliary well 1810 (e.g., which can be used to hold water for rehydration of lyophilized assay reagents), a wash well 1812-1816 (e.g., having a washing solution in which nucleic acids present on magnetic particles are washed); a lysis well 1818 (e.g., having a lysis solution for lysing membranes or walls of the pathogens, infectious agents, and/or cells within the sample to release nucleic acids therein), a pretreatment well 1820 (e.g., which can include a solution including a protease for pretreating a sample prior to lysis); or a well sized to hold a plunger 1806 (e.g., a plunger 1806 that has already been used during the sample preparation process).

In certain embodiments, the plunger 1806 can have a shape that almost entirely fills the volume of the bottom portion of all the wells 1808-1822. This forces the fluid in the wells to be driven up and down with a range large enough to mix the reagents adequately. In certain embodiments, the tip of the plunger 1806 can be fluted in order to provide enough space to ensure the fluid can easily flow up when the plunger is submerged in the liquid during the mixing. According to certain embodiments, the plunger 1806 is a plunger as described in U.S. Patent Application Publication No. 2017/0267996, the disclosure of which is incorporated herein by reference in its entirety.

According to certain embodiments, the systems and methods of the present disclosure include and employ any of the sample preparation (SP) cartridges described in U.S. Patent Application Publication No. 2017/0268038, the disclosure of which is incorporated herein by reference in its entirety. For example, but not by way of limitation, the SP cartridges of the present disclosure can comprise: a frame, comprising: a plurality of wells integrated therewith, wherein the plurality of wells have a closed bottom and an open top; and an opening within the frame having a reaction vessel (RV) or RV cap removably disposed therein, wherein the plurality of wells and the opening are linearly arranged relative to each other.

An SP cartridge elevator of the present disclosure can include a sensor for sensing the presence of an SP cartridge at the top of the elevator. For example, the sensor can be an optical sensor capable of determining the presence or absence of an SP cartridge at the top position of the elevator. When an SP cartridge is not detected at the top position, the system is capable instructing the robotic SP handler to begin retrieving SP cartridges from a different SP cartridge elevator. In certain embodiments, the system is also capable of notifying the operator that the SP elevator should be reloaded.

In certain aspects, the systems of the present disclosure carry out sample preparation in SP cartridges present at one or more positions of a sample preparation station. The SP cartridges can be transported and placed at the positions of the sample preparation station having already been filled with the reagents and samples necessary for the sample preparation process.

In certain embodiments, e.g., as illustrated in FIGS. 46A-46B, the sample preparation station can include one or more sample preparation (SP) modules 2800 In certain embodiments, an SP module 2800 performs the mechanical motions needed to extract nucleic acid. For example, but not by way of limitation, the SP module 2800 can have features that pull the mixing plunger from the cartridge 2802 and 2804 and move it to different wells along the SP cartridge 2802 and 2804 to mix reagents. In certain embodiments, mixing takes place using the plunger 2904 attached to a vertically translating plunger bar. In certain embodiments, the SP module can also have two independently controllable and movable heaters. In certain embodiments, the first heater can be used to heat reagents in the pretreatment and lysis wells. In certain embodiments, the second heater can be used to heat fluid in the eluate well. Both heaters can be engaged during mixing in their respective wells.

In certain embodiments, e.g., as illustrated in FIG. 46B, the SP module can move magnetic microparticles from one well to another by inserting a magnet 2902 into the plunger 2904 which attracts particles to the external wall of the plunger 2904. Once the particles are magnetically attached to the plunger 2904, it is moved to the next well. The particles are released by removing the magnet 2902 from inside the plunger 2904. In certain embodiments, vertical motion of the plunger 2904 by SP module can also cause mixing in the wells.

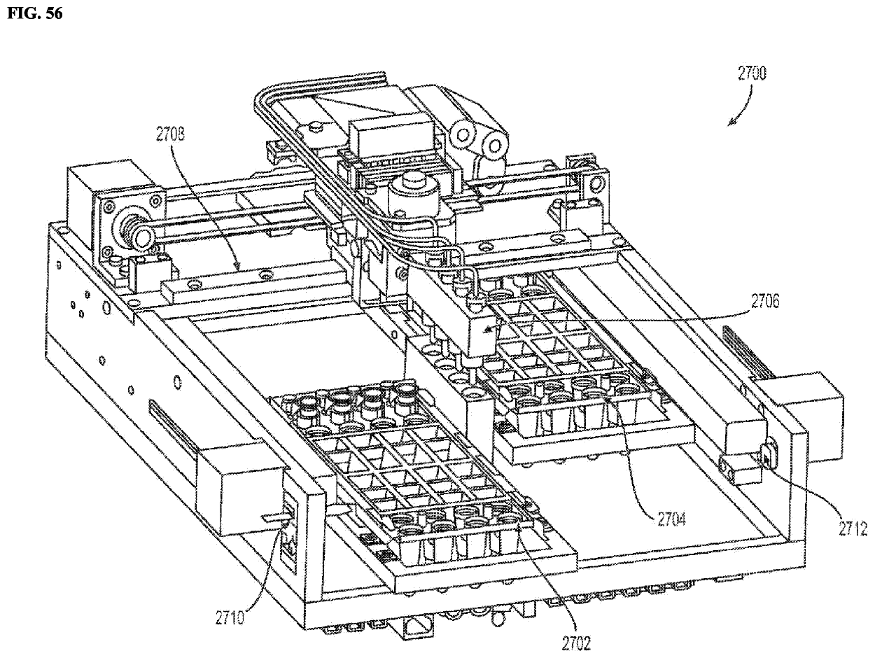

Any suitable approach for filling the SP cartridges prior to sample preparation can be employed. In certain embodiments, e.g., as illustrated in FIG. 54A, an SP cartridge is picked up from the top of an SP cartridge elevator at the SP cartridge loading area (labeled “1” in FIG. 54A) by a robotic SP cartridge handler and transported by the handler to one of two or more positions of an SP cartridge bulk reagent filling station (labeled “2” in FIG. 54A). In certain embodiments, e.g., as illustrated in FIG. 56, the bulk reagents are dispensed into the appropriate wells of SP cartridges 2702 and 2704 at the SP cartridge bulk reagent filling station 2700. In certain embodiments, e.g., as illustrated in FIG. 54A, the robotic SP cartridge handler transports SP cartridges 2702 and 2704 to one of two or more positions of an SP cartridge sample filling station (labeled “3” in FIG. 54A). In certain embodiments, after samples (and optionally, additional reagents) are dispensed into the appropriate wells of SP cartridges 2702 and 2704 at the SP cartridge sample filling station, the robotic SP cartridge handler transports SP cartridges 2702 and 2704 to one of two or more positions of a sample preparation station where sample preparation (e.g., nucleic acid isolation and purification) occurs, e.g., labeled “4” in FIG. 54A. In certain embodiments, the system can include an SP cartridge waste robot that picks up SP cartridges upon completion of sample processing and discards the used SP cartridges in an SP cartridge waste container, e.g., as illustrated in FIG. 54A. Alternatively, or additionally, the waste robot can move the tray in a horizontal or vertical direction to a trash bin area after the samples in the selected tray have been depleted. Additionally, or alternatively, the user can remove the used trays from the trash bin area.

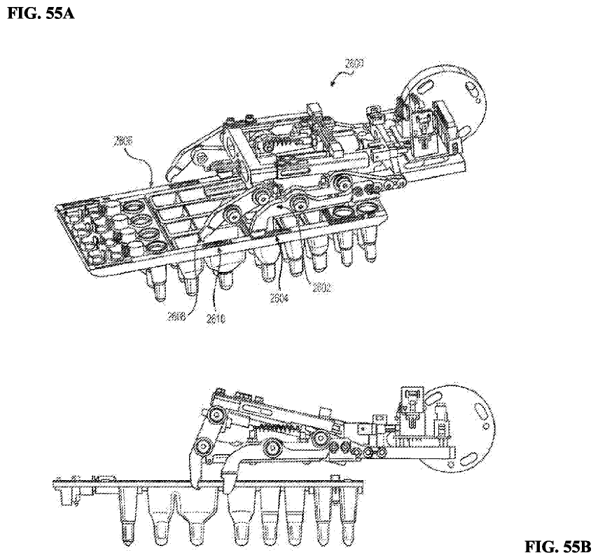

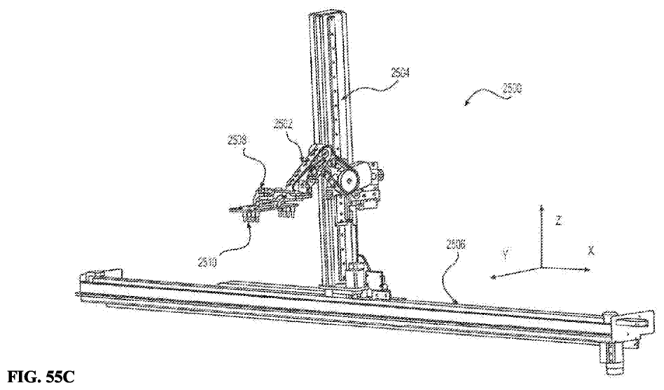

A robotic SP cartridge handler of the present disclosure, e.g., the robotic SP cartridge handler 2500 of FIG. 55A-55C, can have a variety of functions including retrieving SP cartridges 2606 from: an elevator at the SP cartridge loading area, a bulk filling station, a sample filling station and a sample processing station (e.g., positions labeled “1”-“4” of FIG. 54A, respectively). The robotic SP cartridge handler 2500 can also function to distribute SP cartridges 2606 to a bulk filling station, a sample filling station, a sample processing station, and/or a waste robot. The robotic SP cartridge handler 2500 can be designed to permit the robotic pipetting device to work in an adjacent sample preparation unit without interference, provide information to the system indicating whether it has an SP cartridge 2606, determine the presence or absence of an SP cartridge 2606 at any location, and/or the like.

Robotic SP cartridge handlers 2500 of the present disclosure are, in certain embodiments, capable of moving in the X, Y and Z directions. For example, dual link arms can permit movement in the Y axis, e.g., as illustrated in FIG. 55C. In certain embodiments, movement along rails enables movement in the Z and X axes, respectively, e.g., as illustrated in FIG. 55C. SP cartridge handlers 2500 of the present disclosure can also include one or more SP cartridge gripper 2600 for gripping SP cartridges 2606.

In certain embodiments, the SP cartridge grippers 2600 of the present disclosure can include two passive fingers 2602 which are inserted into corresponding slots 2604 of an SP cartridge 2606 by moving down and then forward to engage the passive fingers 2602 with the cartridge 2606. Then, in certain embodiments, two active fingers 2608 are flexed/gripped for insertion of the active fingers 2608 into corresponding slots 2610 of an SP cartridge 2606. In certain embodiments, to complete the grip, the active fingers 2608 are driven towards the passive fingers 2602. Releasing the SP cartridge 2606 at a desired position within the system can be carried out by, in certain embodiments, ungripping the active finger 2608 and removing the active 2608 and passive fingers 2602 from the corresponding slots of the SP cartridge 2606. The robotic SP cartridge handler 2500 can include one or more optical sensors for, e.g., home position, cartridge presence, and/or the like.

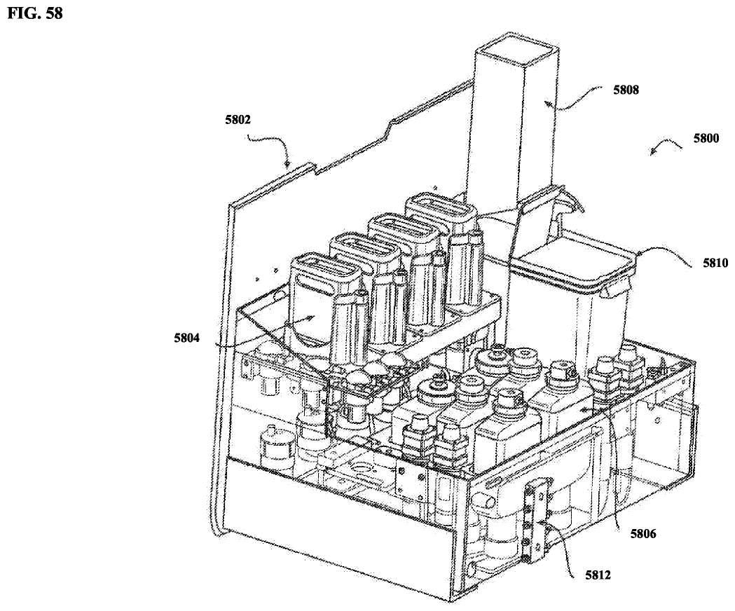

In certain embodiments where the system includes a bulk reagent filling station, e.g., the position labeled “2” of FIG. 54A, such a filling station can include one or more (e.g., 2, 3, 4, or more) SP cartridge bulk reagent filling positions. The bulk reagent filling station can be in fluid communication with the bulk reagent storage area, e.g., the bulk reagent storage area 5800 illustrated in FIG. 58, and dispenses into the appropriate wells of SP cartridges bulk reagents that can be utilized during the sample preparation process, e.g. one or more of molecular grade water, ethanol, lysis reagent, wash reagent, vapor barrier liquid (e.g., an oil), and/or the like.

In certain embodiments, the sample preparation systems of the present disclosure can include an SP cartridge sample filling station, e.g., the position labeled “3” of FIG. 54A. Such a station can include one or more (e.g., 2, 3, 4 or more) SP cartridge sample filling positions. In certain embodiments, for sample filling, the robotic pipettor, e.g., the robotic pipettor 3114 of FIG. 54A, aspirates samples from sample tubes present at the internal portion of sample loading area, e.g., the sample loading area 3102 of FIG. 54B, is transported to the sample filling station, and dispenses samples into the appropriate wells of SP cartridges present at the one or more SP cartridge sample filling positions of the sample filling station.

According to certain aspects, the methods of the present disclosure include transporting an SP cartridge from the SP cartridge loading area to one of two or more positions of the SP cartridge bulk reagent filling station. In certain aspects, the methods include transporting an SP cartridge from the SP cartridge bulk reagent filling station to one of two or more positions of the SP cartridge sample filling station. The methods can include transporting an SP cartridge from the SP cartridge sample filling station to one of two or more sample preparation positions of the sample preparation station, e.g., the sample preparation station labeled “4” of FIG. 54A. Any (e.g., each) of the above transporting steps can be performed by the robotic SP cartridge handler, e.g., the robotic SP cartridge handler 2500 of FIG. 55.

In addition, or as further alternatives, and as embodied herein, sample preparation processes as described herein can include operations where particles or beads are passed through the surface of a liquid and/or through an air-aqueous or oil-aqueous boundary. For example, only and not limitation sample preparation processes for use with the disclosed subject matter can include or incorporate EXTRACTMAN™, SLIDE™ or AirJump™ sample preparation-based aspects and/or systems (each by Salus Discovery, LLC), including aspects disclosed in U.S. Pat. Nos. 8,603,416, 10,040,062, and 10,441,950, each of which is incorporated herein by reference in its entirety.

### 6.2 Target Nucleic Amplification Systems

In accordance with another aspect of the disclosed subject matter, the HTNAT systems of the present disclosure includes an amplification system configured to facilitate the amplification of the target nucleic acid. For example, but not by way of limitation, the amplification system will comprise reaction vessels into which nucleic acid isolated via the sample preparation methods and systems described herein have been transferred. In certain embodiments, the reaction vessels will traverse a plurality of positions within the amplification system, e.g., reagent addition positions, heating positions, and/or cooling positions. In other embodiments, the reaction vessels are essentially stationary, and the reagent addition is accomplished via, e.g., robotic pipettors, and the heating and cooling can be accomplished via heating and/or cooling localized to the stationary position of the reaction vessel.

### 6.2.1 Carousel-Based Nucleic Acid Amplification Systems

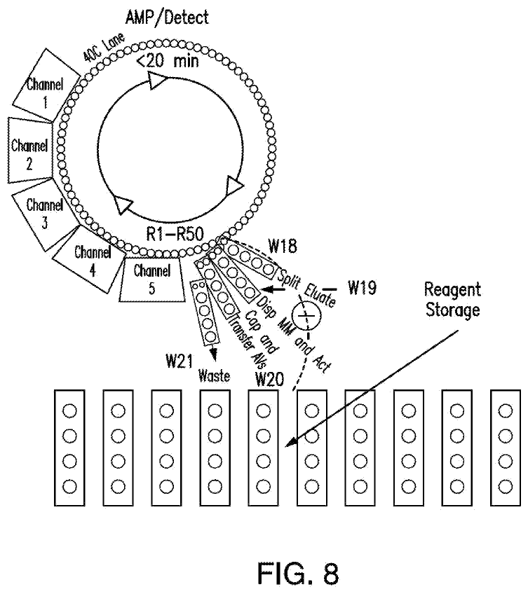

As embodied herein, and in accordance with another aspect of the disclosed subject matter, an exemplary Amplification system according to the disclosed subject matter is the combined Amplification/Detection system (also referred to herein as an amplification and detection system). Exemplary Amplification/Detection systems are depicted in FIG. 8 and as 6650 in FIG. 66. Generally, the combined Amplification/Detection system amplifies the target to obtain a detectable signal and detects the signal. As embodied herein, the amplification process can be any of a variety of strategies to amplify the target nucleic acid. For example, but not by way of limitation, the amplification process can employ isothermal amplification. As discussed in detail, above, non-limiting examples of isothermal amplification operations useful in connection with the disclosed subject matter include RPA, NEAR, and Transcription Mediated Amplification (“TMA”), as well as any other suitable isothermal amplification technique.

The below table, Table 8, depicts exemplary times and operations regarding the amplification and detection process of FIG. 66. In certain embodiments, the amplification and detection system is a rotatable carousel having positions to hold amplification vessel. For example, as shown in FIG. 66 for illustration and not limitation, the amplification and detection system 6650 is a rotatable carousel 6651 having positions to hold amplification vessel 6660. The amplification vessel 6660 holds the amplification reagents and eluate during the amplification and detection process. In the embodiment of FIG. 66, the carousel has 107 positions (R1-R106). Not all positions on the carousel 6651 are used in the sample amplification process in that some position are used to load the amplification vessel 6660 onto the carousel 6651, dispense amplification reagents or remove the amplification vessel. As shown in this exemplary embodiment using a lockstep of 12 seconds, the sample processing time for the amplification and detection process is 1224 seconds, or 20.4 minutes.

TABLE 8

| | | Sample Process |
|---|---|---|
| Pos. | Function | Time (Seconds) |
| R1 | Load Amp Vessel Cap | |
| R2 | Load Amp Vessel | |
| R3 | No operation | |
| R4 | Dispense Activator to Amp Vessel | |
| | (3-8 μl +/− 5%; assay specific volume) | |
| R5 | Dispense Eluent to Amp Vessel | 12 |
| | (3-8 μl +/− 5%; assay specific volume) | |
| | 20 μl +/− 5%) | |
| | Wash Probe | |
| | Seal Amp Vessel | |
| R6 | Dispense MasterMix to Amp Vessel | 12 |
| | (10 μl +/− 5%; 15 μl +/− 5%, | |
| | 30 μl +/− 5%) | |
| | Wash probe | |
| | Seal Amp Vessel | |
| R7 | Vigorous Mixing (@12 seconds) | 12 |
| R8- | Incubation (40° C.) | 1188 |
| 106 | Continuous Reading of 5 channels | |
| | every second lockstep | |
| | Indexing | |
| [R31] | [Additional Vigorous Mixing Step] | |
| R107 | Transfer Amp Vessel to Waste | |

FIG. 66 includes positions R1-R107, a subset of which are positioned relative to one or more fluorescent readers 6663, but which also function as amplification positions. Exemplary position R3 of FIG. 66 corresponds to the position where an activator is dispensed into, e.g., an amplification vessel, e.g., via “Sip & Spit” strategy from a reagent container. In certain embodiments, the activator is a material, e.g., an enzyme or cofactor, that initiates the amplification reaction.

Elute is then transferred to the amplification and detection system of FIG. 66. In one embodiment, about 40 μl of eluate is aspirated from P4 of a wash vessel 6540 the wash and elution system 6530. As mentioned above, in the embodiment of FIG. 65, the lockstep is 12 seconds, wherein the carousel indexes one position forward at each lockstep. In this embodiment, at position R5, about 20 μL of the eluate is the dispensed into an amplification vessel 6660 located on the amplification and detection carousel 6651. That amplification vessel is indexed to position R6 wherein at R6, the amplification reagents, sometimes referred to as a “MasterMix” are dispensed into the amplification vessel and the amplification vessel is capped. At the same time, the other about 20 μl of eluate is dispensed into another amplification vessel at R5. After the lockstep, the amplification vessel is indexed to R6 wherein amplification reagents are added and the vessel is capped. As will be described in more detail below, after the second portion of the eluate is capped, the carousel rapidly rotates 360 degrees within the same lockstep so that each amplification vessel on the carousel can be read by one or more detectors every 24 seconds. By rotating the carousel 360 degrees every other 12 second lockstep (i.e., every 24 seconds), only capped amplification vessels are rotating thus reducing the chance of amplicon from contaminating the reaction carousel. Capping of the amplification vessels can be done, e.g., via use of a press on cap, heat stake tape, and/or PSA tape.

Positions R8-R106 are employed, in certain embodiments, to achieve an amplification incubation with, in certain embodiments, continuous mixing. In certain embodiments, the duration of the amplification incubation is about 1188 seconds. In certain embodiments, the R8-R106 incubation occurs at 40° C., where heating is performed via, in certain embodiments, resistive heaters. Continuous mixing at positions R8-R106 can be performed, in certain embodiments, via carousel movement and/or pop-up mixers. In certain embodiments, one or more positions, e.g., positions, including but not limited to, positions R7 and R31, are sites of vigorous mixing, e.g., mixing via pop-up mixer.

The below table, Table 8.1, depicts exemplary times and operations regarding the amplification and detection process. As shown in this exemplary embodiment using a lockstep of 12 seconds, the sample processing time for the amplification and detection process is 1200 seconds, or 20 minutes.

TABLE 8.1

| | | Sample Process |
|---|---|---|
| Pos. | Function | Time (Seconds) |
| R1 | Load Amp Vessel Cap | |
| R2 | Load Amp Vessel | |
| R3-R12 | | |
| R13 | Dispense Activator | |
| R14 | | |
| R15 | Dispense Master Mix | |
| | Wash Probe | |
| R16 | Dispense Eluent to Amp Vessel | 12 |
| | Seal Amp Vessel | |
| R17 | Mixing and Incubation | 12 |
| | Continuous Reading of 5 Channels | |
| R18-R40 | Incubation | 276 |
| R41 | Incubation | 12 |
| | Vigorous Mixing (@ 300 seconds) | |
| R42-R116 | Incubation | 900 |
| R117 | Transfer Amp Vessel to Waste | 12 |

In the above exemplary system, where a lockstep of 12 seconds is used, the sample is in the amplification and detection process for 100 locksteps (R17-R116), which results in a total incubation time for the amplification and detection process of about 1200 seconds, or about 20 minutes.

In certain embodiments, the processing time of the sample for the amplification and detection process starts with the start of incubation in a vessel of the amplification and detection system (e.g., exemplary position R17) and stops with the end of incubation in the amplification and detection system (e.g., exemplary position R116). In certain embodiments, the total sample processing time in the amplification and detection process can be from about 1 minute to about 22 minutes, about 5 minutes to about 22 minutes, 18 minutes to about 22 minutes, about 19 minutes to about 21 minutes, or about 20 minutes to about 21 minutes. In certain embodiments, the total sample processing time in the amplification and detection process can be about 1 minute, about 5 minutes, about 10 minutes, about 11 minutes, about 12 minutes, about 13 minutes, about 14 minutes, about 15 minutes, about 16 minutes, about 17 minutes, 18 minutes, about 19 minutes, about 20 minutes, about 21 minutes, or about 22 minutes. In particular embodiments, the total sample processing time in the amplification and detection process can be about 1200 seconds, or about 20 minutes, or about 1224 seconds, or about 20.4 minutes.

Alternatively or additionally, the processing time of the sample for the amplification and detection process starts with the start of incubation of the sample in a vessel of the amplification and detection system (e.g., exemplary position R17) and ends with the determination of a result. In certain embodiments, the result is the detection of one or more target nucleic acids derived from at least one pathogen or infectious agent in excess of a predetermined level.

In certain embodiments, the amplification and detection process can have a duration of about 1 minute or less. For example, but not by way of limitation, the amplification and detection of a nucleic acid of a pathogen or infectious agent present in the sample at high titers can occur at about 1 minute after the start of incubation in a vessel of the amplification and detection system. In certain embodiments, high titers of a pathogen or infection agent can include titers of at least 1×10¹² IU/ml. For example, but not by way of limitation, a sample that has a pathogen or infectious agent titer of at least 1×10¹² IU/ml or more can be detected in about 1 minute or less after the start of incubation in a vessel of the amplification and detection system. In certain embodiments, the detection of a level that is in excess of a predetermined level can be obtained after about 1 minute or less into the amplification and detection process for samples with high titers.

In certain embodiments, the amplification and detection process can have a duration of about 5 minutes or less. For example, but not by way of limitation, the amplification and detection of a nucleic acid of a pathogen or infectious agent present in the sample at medium titers can occur at about 5 minutes or less after the start of incubation in a vessel of the amplification and detection system. In certain embodiments, medium titers of a pathogen or infection agent can include titers of at least 1×10⁶ IU/ml. For example, but not by way of limitation, a sample that has a pathogen or infectious agent titer of at least 1×10⁶ IU/ml or more can be detected in about 5 minutes or less after the start of incubation in a vessel of the amplification and detection system. In certain embodiments, the detection of a level that is in excess of a predetermined level can be obtained after about 5 minutes or less into the amplification and detection process for samples with medium titers.

In certain embodiments, the amplification and detection process can have a duration of about 20 minutes. For example, but not by way of limitation, the amplification and detection of a nucleic acid of a pathogen or infectious agent present in the sample can occur at about 20 minutes after the start of incubation in a vessel of the amplification and detection system. In certain embodiments, to confirm that a nucleic acid of a pathogen or infectious agent is not present in the sample or presence at levels lower than a predetermined amount, the amplification and detection process can have a duration of about 20 minutes.

In certain embodiments, the amplification and detection process can have a duration of about 20 minutes. For example, but not by way of limitation, determining that a nucleic acid of a pathogen or infectious agent is not present in the sample can occur at about 20 minutes after the start of incubation in a vessel of the amplification and detection system. Alternatively, the determination that a nucleic acid of a pathogen or infectious agent is present in the sample in excess of a predetermined level can be obtained within 1 minute from the start of incubation in a vessel of the amplification and detection system. In certain embodiments, the determination that a nucleic acid of a pathogen or infectious agent is present in the sample in excess of a predetermined level can be obtained within 5 minutes from the start of incubation in a vessel of the amplification and detection system.

For TTR calculations, the processing time of the sample in the amplification and detection process does not include loading the amplification/detection vessel cap (e.g., exemplary position R1), loading the amplification/detection vessel (e.g., exemplary position R2), dispensing the activator (e.g., exemplary position R13), dispensing the eluent to the amplification/detection vessel (e.g., exemplary position R16), transferring the amplification/detection vessel to waste (e.g., exemplary position R117), and any positions with no operation (e.g., exemplary positions R3-R12 and R14).

For purpose of illustration and not limitation, an exemplary Amplification system according to the disclosed subject matter is the combined Amplification/Detection system (e.g., amplification and detection system) depicted in FIG. 7 and FIG. 8. In exemplary FIGS. 7-8, position W19 corresponds to the position where the MasterMix, described above, is dispensed into first and second amplification vessels, e.g., via “Sip & Spit” strategy from a reagent container.

Exemplary position W19 of FIG. 8 corresponds to the position where an Activator is dispensed into, e.g., first and second amplification vessels, e.g., via “Sip & Spit” strategy from a reagent container. In certain embodiments, the Activator is a material, e.g. an enzyme or cofactor, that initiates the amplification reaction. Exemplary position W20 also functions to seal the exemplary first and second amplification vessels, e.g., via use of a press on cap, heat stake tape, and/or PSA tape. Position W20 can also allow for transfer of the first and second amplification vessels into the detection/read carousel, e.g., by use of a “press down/break frangible tab” and/or a “Pick & Place” strategy. Alternative strategies for transfer of the sample from position W20 to the detection/read carousel include using a separate vessel and cap resting within locations of the wash vessel, or separate vessels and caps loaded in various racks, which can be Picked & Placed into the proper location as required

FIG. 8 includes positions W18-W20 (see description of FIG. 7, above) as well as detection (“reading”) positions R1-R50, a subset of which are positioned relative to one or more readers, but which also function as amplification positions, and waste position R51. For example, positions RT-R50 are employed, in certain embodiments, to achieve an amplification incubation with, in certain embodiments, continuous mixing. In certain embodiments, the duration of the amplification incubation is about 1200 seconds. In certain embodiments, the RT-R50 incubation occurs at 40° C., where heating is performed via, in certain embodiments, resistive heaters. Continuous mixing at positions RT-R50 can be performed, in certain embodiments, via carousel movement and/or pop-up mixers. In certain embodiments, one or more positions, e.g., positions from RT-R25, including but not limited to, position R12, is a site of vigorous mixing, e.g., mixing for 288 seconds via pop-up mixer.

In certain embodiments, the combined Amplification/Detection system will rotate every amplification vessel past the readers each index period within the lockstep, e.g. achieving a 360 degree+one position movement in each index period. In certain embodiments, the lockstep is every 24 seconds, although different locksteps can be used. In certain embodiments, the index period to complete the movement of the carousel is about 0.5 to about 2 seconds. In alternative embodiments the lockstep and/or index period can be reduced or increased, depending on throughput and TTR requirements.

In certain embodiments, the centrifugal force created during rotation of the combined Amplification/Detection system is taken advantage of to enhance sample processing. Depending on the final carousel diameter and gear ratios between the carousel and motor, one can rotate this carousel at about 600 RPM and produce approximately about 50 RCF (G forces) at each amplification vessel. This centrifugation force would, in certain embodiments, cause droplets on the side or top cover of the amplification vessel to return to the bottom (e.g., if the amplification vessels are allowed tilt during rotation). Droplets can be generated by vigorous mixing and/or combined Amplification/Detection system rotation. By returning any droplets to the bottom (read area) of the amplification vessel, the signal from the volume read and its integrity can be maximized.

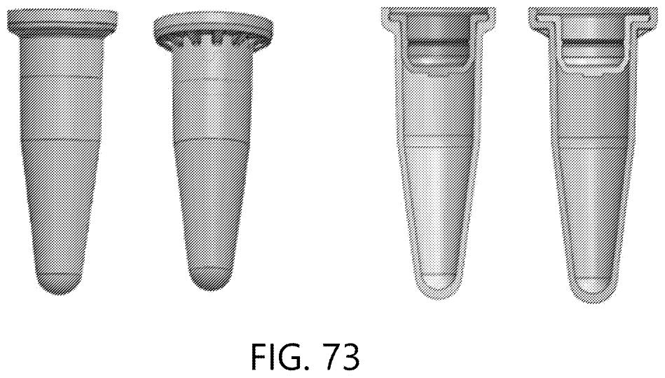

FIG. 73 depicts exemplary amplification vessels for use in the systems as disclosed herein. As embodied herein, the amplification vessels can be pre-loaded with reagents and/or enzymes. The amplification vessels are disposed into the amplification and detection system 6807. As embodied herein, the amplification vessels can have atop flange with a narrower bottom and can have caps for sealing. The amplification vessels can be sealed with the caps in less than 2 seconds after the eluate addition. For purpose of illustration not limitation, the caps for the amplification vessels can be press fit. As embodied herein, the caps can be made of medical-grade copolymer PP.

The below table, Table 9, depicts exemplary times and operations regarding the amplification and detection process of FIG. 8. As shown in this exemplary embodiment using a lockstep of 24 seconds, the sample processing time for the amplification and detection process is 1200 seconds, or 20 minutes.

TABLE 9

| | | Sample Process Time |
|---|---|---|
| Pos. | Function | (Seconds) |
| W18 | Dispense MasterMix to Amp Vessel | |
| W19 | Dispense Activator to Amp Vessel | |
| W20 | Seal Amp Vessel | |
| | Transfer Amp Vessels to Reading | |
| | Carousel | |
| R1- | Incubation (40° C.) | 1200 |
| R50 | Continuous Mixing | |
| R12 | Vigorous Mixing (@288 seconds) | |
| | Indexing | |
| | Reading | |
| R51 | Transfer Amp Vessel to Waste | |

### 6.2.2 Additional Target Nucleic Acid Amplification System Embodiments

Although some of the embodiments of the amplification vessels described herein are individual and are processed individually, the present invention also contemplates amplification (or lysis, or wash, etc.) vessels that are formed in a plurality. For example, FIG. 54D is an example of amplification (or lysis, or wash etc.) that are processed as a plurality. In the example in FIG. 54D, the five vessels are connected with each other and can be processed as a plurality.

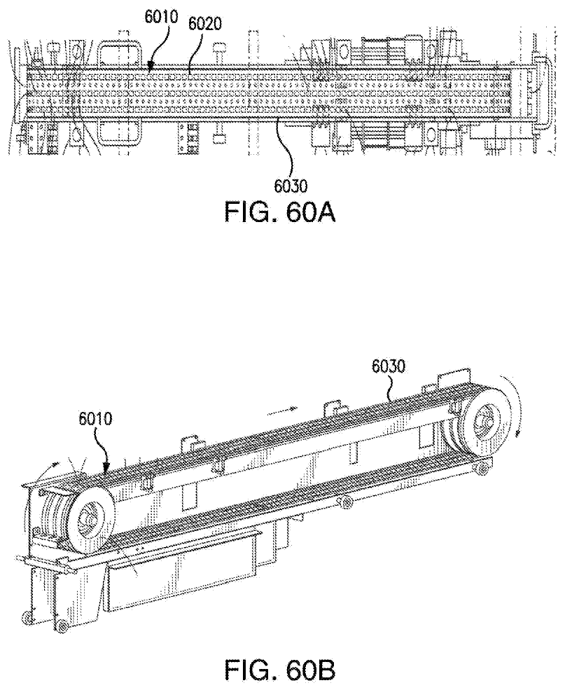

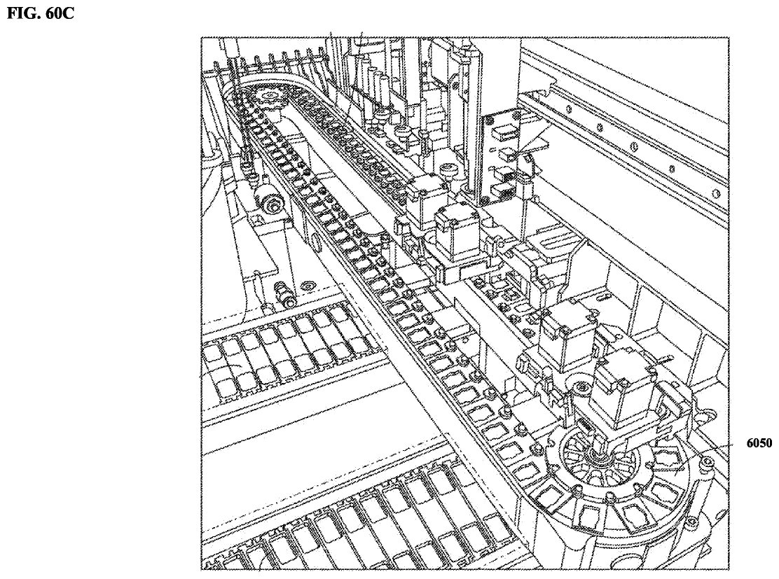

Additionally, or alternatively, and in accordance with another aspect of the disclosed subject matter, the amplification systems of the present disclosure can comprise reactions 6010 that traverse positions, e.g., sample processing paths, on tracks (FIGS. 60A-60C).

Additionally, or alternatively, amplification vessels 6010 are placed into spaced-apart incubation track slots 6020 of the track 6030 (FIGS. 60A-60B). The amplification vessels are operable to contain a sample and one or more reagents for carrying out one or more amplification reaction. In certain embodiments, a processor can control the incubation track to move, causing the reaction vessels residing in the incubation track to advance the reaction vessels to their next location within the amplification system.

In certain embodiments, the at least one processor can control the incubation track to cause the amplification vessels to move through a plurality of locations to allow the sample and reagent in the amplification vessels to amplify and complete a desired reaction. The track will move at a speed such that the amplification vessel will reside in the amplification track of the amplification process path so as to allow the desired amplification time.

With regard to FIG. 60C specifically, additionally, or alternatively, additional processing steps, including amplification, can be performed on an auxiliary track 6050 wherein vessels related to nucleic amplification reactions, or the contents of those vessels, can be transferred from the process path of auxiliary track 6050 to the process path of track 6030. As will be appreciated by those in the art, the configuration of the auxiliary track(s) and the amplification track(s) can vary.

In certain embodiments, the processing track(s) are formed as a continuous linear belt-like track that is disposed around pulleys (FIG. 60). In certain embodiments, the pulleys can engage the processing track(s) in a sprocket-wheel engagement, in a friction engagement, or other forms of engagement to cause translation or movement of the processing track(s) (FIG. 60). In certain embodiments, a motor supplies power to one or more of the pulleys in order to rotate the pulleys. In certain embodiments, the rotation of the pulleys causes the interfaced processing track(s) to rotate with and around the pulleys, thereby moving the processing track(s) simultaneously.

Although the above description provides an example of the traversal of reaction vessels through various locations, it is noted that, in certain embodiments, the components of the amplification system can vary, the components of the amplification system can be oriented or configured in different locations, or one or more additional components can be added to the amplification system, and one of skill in the art would understand that such modifications would involve reciprocal modifications of the methods and systems involved in traversal of the reaction vessels through various locations.

As was described in detail with respect to the sample preparation systems of the present disclosure, the amplification systems of the present disclosure can, in certain embodiments, make use of, e.g., pipette tip loading areas, bulk fluid storage areas, and liquid and solid waste storage areas configured as described herein.

Additionally, or alternatively, and in accordance with another aspect of the disclosed subject matter, the amplification systems of the present disclosure can comprise a processing deck upon which amplification vessels can be located, e.g., a sample analysis station 3112 as illustrated in FIG. 54B. In certain embodiments, the processing deck will be surrounded by a housing that allows a plurality of assembly rings, a support structure cantilevered over, or a support structure fixed over the processing deck, e.g., as illustrated in FIG. 54B. In certain embodiments, the housing or support structure can be configured to position one or more robotic pipettors capable 3114 of introducing or aspirating fluid into the amplification vessels located on the processing deck. In certain embodiments, addition assembly rings or support structures can be configured to hold amplification reagents and/or disposal receptacles. In certain embodiments, the processing deck will include a plurality of heating and/or cooling units configured to facilitate the heating and cooling of the amplification vessels. Additionally, or alternatively, such heating and/or cooling units can be incorporated into a partial or fully enclosed subsystem in which amplification reactions occur. For example, but not limitation, the partially or fully enclosed subsystem is a thermocycler as described in U.S. Pat. No. 7,148,043, the contents of which are incorporated by reference in their entirety. In certain embodiments, partially or fully enclosed subsystem can comprise a support structure comprising one or a plurality of wells. The support structure can be positioned such that the heating and/or cooling units are able to heat and/or cool amplification vessels when such vessels are supported by the support structure. For example, but not limitation, the support structure can be designed to hold one or a plurality of amplification vessels that can be transferred individual or collectively, e.g., in a 96 well plate that aligns with the wells of the wells of the support structure, to facilitate amplification. In certain embodiments, the heating and/or cooling subsystem can incorporate fixed or movable detection systems, as described in detail herein. In certain embodiments, the processing deck can comprise a mixing unit configured to facilitate mixing of the amplification vessels. Additionally, or alternatively, the amplification system can include incubators, e.g., rotary incubators, configured to maintain the amplification vessels at a predetermined temperature for a predetermined length of time.

### 6.3 Target Nucleic Acid Detection Systems

In accordance with another aspect of the disclosed subject matter, the HTNAT systems of the present disclosure include a detection system configured to facilitate the detection of the target nucleic acid. For example, but not by way of limitation, the detection system can comprise reaction vessels where washed and eluted nucleic acid have been contacted with the materials necessary to amplify a target nucleic acid. In certain embodiments, the reaction vessels will traverse a plurality of positions within the detection system, e.g., one or more readers. In other embodiments, the reaction vessels are essentially stationary, and the detection is accomplished via detectors that can be movably, e.g., robotically, localized to the stationary position of the reaction vessel. Additionally, or alternatively, in systems employing stationary amplification vessels, including but not limited amplification systems comprising partial or fully enclosed heating and/or cooling subsystems, the detection systems of the present disclosure can incorporate fixed readers, e.g., incorporated into the housing of a partially or fully enclosed heating and/or cooling subsystem.

### 6.3.1 Carousel-Based Detection Systems

For purpose of illustration and not limitation, an exemplary amplification system according to the disclosed subject matter is the combined Amplification/Detection system. For example, but not by way of limitation, an exemplary amplification system is the combined Amplification/Detection system, i.e., the amplification and detection system, depicted in FIG. 66. Generally, the combined amplification/detection system amplifies the target to obtain a detectable signal and detects the signal. As embodied herein, the amplification operation can be any of a variety of strategies to amplify the target nucleic acid. For example, but not by way of limitation, the amplification operation can employ isothermal amplification. As discussed in detail, above, non-limiting examples of isothermal amplification operations useful in connection with the disclosed subject matter include RPA, NEAR, and Transcription Mediated Amplification (“TMA”), as well as any other suitable isothermal amplification technique. In certain embodiments, the isothermal amplification reaction is RPA. In certain embodiments, the isothermal amplification reaction is NEAR.

An exemplary Detection system according to the disclosed subject matter is the combined Amplification/Detection system depicted in FIG. 66. As described above, in certain embodiments, the combined amplification/detection system can rotate every amplification vessel past the readers every other lockstep (i.e., every 24 seconds) e.g., achieving a 360 degree in every second lockstep period. In certain embodiments, the lockstep is every 12 seconds, although different locksteps can be used. In alternative embodiments the lockstep and/or index period can be reduced or increased, depending on throughput and TTR requirements.

In certain embodiments, the centrifugal force created during rotation of the combined Amplification/Detection system is taken advantage of to enhance sample processing. Depending on the final carousel diameter and gear ratios between the carousel and motor, one can rotate this carousel at about 600 RPM and produce approximately about 50 RCF (G forces) at each amplification vessel. This centrifugation force would, in certain embodiments, cause droplets on the side or top cover of the amplification vessel to return to the bottom (e.g., if the amplification vessels are allowed tilt during rotation). Droplets can be generated by vigorous mixing and/or combined Amplification/Detection system rotation. By returning any droplets to the bottom (read area) of the amplification vessel, the signal from the volume read and its integrity can be maximized.

An alternative exemplary Detection system according to the disclosed subject matter is the combined Amplification/Detection system depicted in FIG. 8. As noted above, FIG. 8 includes positions W18-W21 (see description of FIG. 7, above) as well as detection (“reading”) positions R1-R50, a subset of which are positioned relative to one or more readers, e.g., fluorometers. In certain embodiments, the system and methods can employ, one, two, three, four, five, six, seven, eight, nine, ten, or more readers. In certain embodiments, the system employs five readers. In certain embodiments, the system employs six readers. Each of said readers can be calibrated to detect a specific signal, e.g., a specific fluorescence signal. In certain embodiments each reader is calibrated to detect a different signal or signal range. In certain embodiments, each signal can be associated with a particular analyte of interest, e.g., an internal control or a target sequence. Embodiments of the present disclosure can comprise greater or fewer than the positions identified in FIG. 8, depending on alternative system configurations. For purpose of illustration not limitation, with reference to FIG. 72, the amplification and detection system 6807 can include five fluorescent readers 6832 at different wavelengths to conduct optical detection of the amplification vessels.

### 6.3.2 Additional Target Nucleic Acid Detection System Embodiments

6.3.2(a) Detection Systems with Stationary Sample/Moving Reader

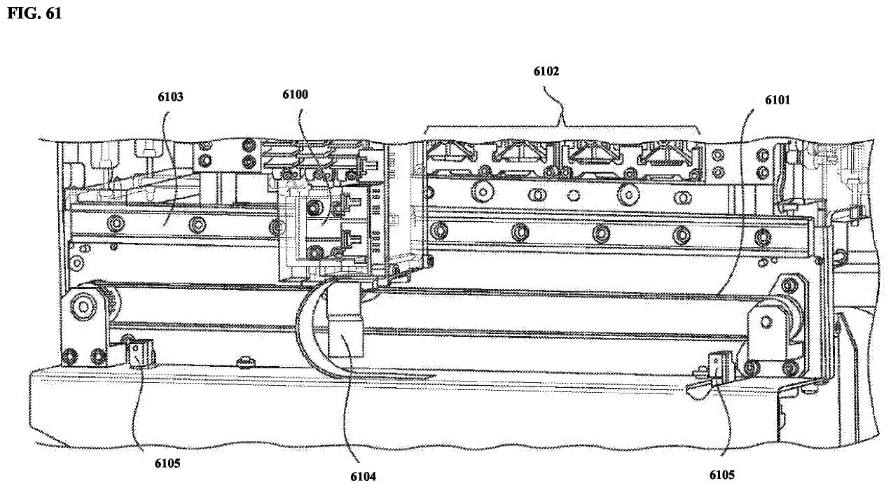

Additionally, or alternatively, and in accordance with another aspect of the disclosed subject matter, the Detection systems of the present disclosure can incorporate readers capable of shuttling across a plurality of positions, e.g., gantry readers or slidable optics, to be in optical communication with stationary samples (e.g., amplification reaction vessels), e.g., as embodied by the exemplary traveling reader 6100 illustrated in FIG. 61. For example, but not by way of limitation, a fluorescence detection apparatus for analyzing samples located in a plurality of reaction vessels 6102, e.g., tubes, wells, etc., can comprise: a support structure 6103 attachable to amplification system comprising stationary reaction vessels; a shuttle movably mounted on the support structure 6103; and one or more reader 6100. Additionally, or alternatively, the system can include one or more control and/or position monitoring elements including a flag 6104 and flag sensors 6105. Such flag and flag monitoring components provide a means of, e.g., during automation, providing a processor with the location of the reader along the conveyor or generating other useful data including, e.g., the speed of travel of the optical analysis device, etc. Additionally, or alternatively, the location, speed of travel, and other information relating to the movement of the optical analytical device can be monitored using a suitable absolute encoder. Additionally, or alternatively, the reader 6100 will include: a housing having an opening oriented toward the plurality of reaction vessels 6102; an excitation light generator disposed within the housing; and an emission light detector disposed within the housing.

Additionally, or alternatively, shuttling of the readers 6100, e.g., gantry readers, can be driven by stepper motors. Additionally, or alternatively, the Detection systems of the present disclosure can comprise an x-axis stepper motor and/or a y-axis stepper motor. In certain embodiments, the stepper motors and readers can be designed as known in the art. Additionally, chain drives, belt drives, or other drive mechanisms can be used to position the readers, as illustrated in FIG. 61. Additionally, or alternatively, shuttling of the readers 6100, e.g., gantry readers, can be driven by servo motors.

Additionally, or alternatively, the reader 6100 will be in electronic communication with the other components of the Detection system. In certain embodiments, such communication allows for information relay between the reader and the Detection system as a whole. For example, but not by way of limitation, the information relayed from the Detection system is relayed to a BECS system to facilitate processing, e.g., release, of a donor sample. In certain embodiments, such detection information is combined with additional donor sample information, e.g., blood type, HLA tissue type, and/or data obtained via a donor questionnaire, and relayed to a BECS systems to facilitate processing, e.g., release, of a donor sample.

6.3.2(b) Detection Systems with Moving Sample/Stationary Reader

Additionally, or alternatively, and in accordance with another aspect of the disclosed subject matter, the detection systems of the present disclosure can incorporate amplification reaction vessels that traverse a plurality of positions to thereby enter into optical communication with one or more stationary readers, e.g., the readers identified as Channels 1-5 in FIG. 8 or the readers 6663 of FIG. 66. For example, but not by way of limitation, the detection systems can comprise at least one detection track, e.g., the carousel 6651 of FIG. 66, that is moved to move the amplification reaction vessels held therein to at least one reader 6663. Upon movement of the amplification reaction vessel to the reader 6663, readings are taken of the nucleic acids contained in the reaction vessels.

Additionally, or alternatively, the reader will be in electronic communication with the other components of the Detection system to allow for information relay between the reader and the Detection system as a whole. In certain embodiments, the information relayed from the Detection system is relayed to a BECS system to facilitate processing, e.g., release, of a donor sample.

### 6.3.2(c) Detection Systems for Quantitative Analyses

Additionally, or alternatively, and in accordance with another aspect of the disclosed subject matter, the Detection systems of the present disclosure are capable of performing a plurality of reads during the course of one amplification reaction. For example, but not limitation, the Detection system will rotate every amplification vessel past the readers each index period within the lockstep, e.g., achieving a 360 degree movement in each index period in certain embodiments or, in alternative embodiments, a 360 degree+one position movement in each index period. In certain embodiments, the lockstep is every 12 seconds, although different locksteps can be used, e.g., every 24 seconds. In certain embodiments, the index period to complete the movement of the carousel is about 0.5 to about 2 seconds. In alternative embodiments the lockstep and/or index period can be reduced or increased, depending on throughput and TTR requirements. An index period can generate, in the above embodiment, about 50 individual reads from each detector corresponding to each amplification vessel during an exemplary RPA amplification process. While this particular embodiment describes the use of a carousel-based Detection systems, alternative systems employing movable readers and stationary amplification vessels, as well as alternative systems employing stationary readers and moveable amplification vessels, can also be used to obtain a plurality of reads over the course of a single amplification reaction. As outlined below, the ability to obtain a plurality of reads over the course of an amplification reaction provides the necessary data to obtain a quantitative result.

In certain embodiments, the Detection systems of the present disclosure can perform a plurality of reads during the course of one amplification reaction (also referred to herein as an amplification and detection process). During the amplification reaction, in certain aspects, the time to the first determination of a quantitative result from the initiation of the amplification reaction can be about 1 minute or less, about 5 minutes or less, about 10 minutes or less, about 15 minutes or less, about 20 minutes or less, or about 25 minutes or less. In certain embodiments, the time to the determination of the first quantitative result from the initiation of the amplification reaction can be from about 1 minute to about 25 minutes, from about 1 minute to about 15 minutes, or from about 10 minutes to about 25 minutes. In certain embodiments, the time to the determination of the first quantitative result from the initiation of the amplification reaction can be about 1 minute, about 5 minutes, about 10 minutes, about 15 minutes, about 20 minutes, or about 25 minutes. In certain embodiments, a plurality of reads can be performed by the Detection systems of the present disclosure to provide the first determination of the quantitative result. In certain embodiments, at least one, at least two, at least three, at least four, at least five, or at least ten reads can be used to detect amplification of a sample. For example, but not limitation, the time to the determination of the first quantitative result can include at least two reads, e.g., one for a baseline and one to detect amplification of a target nucleic acid, and provide the determination of the first quantitative result in about 1 minute or less. In certain aspects, a sample with a relatively high titer content can be detected in less time than a sample with a relatively low titer content.

For example, but not limitation, in order to perform quantitative amplification of Parvovirus B19, serial dilutions of positive plasma samples were subject to sample preparation to isolate the nucleic acid. Following sample preparation, RPA reactions were set up as described herein, see e.g., Example 15. This testing was required to determine if the RPA reaction demonstrated linearity and formulation changes were made (e.g., enzyme concentrations) to allow for greater separation between target levels and improved linearity as outlined in Example 15. For quantitation, the time at which the amplification signal crossed a designated threshold (Cycle Threshold, Ct) is measured during the reaction. This is then compared to the signal of samples at known concentrations (Calibrators). The concentration of the unknown sample can then be determined when compared to the Ct of the calibrators.

While described herein specifically with respect to the detection of Parvovirus B19, by using the Detection systems discussed herein, particularly those that allow for a plurality of readings of the course of a single amplification reaction, in combination with a comparison to signals associated with known standards, it is possible to extend this quantitative strategy to the analysis of other pathogens and infectious agents

### 6.4 Auxiliary Systems

### 6.4.1 Random Access Systems

In accordance with another aspect of the disclosed subject matter, the systems of the present disclosure can provide random access to all samples and nucleic acid analyses, meaning that the system permits the ordering and processing of any sample and/or any nucleic acid analysis in any order provided that the system has the necessary reagents/consumables for the requested nucleic acid analysis. For example, but not by way of limitation, because the systems of the present disclosure do not require batch processing, the systems allow for the prioritization of samples, e.g., allowing for “stat” samples to be prioritized over samples already in a queue. This is a significant improvement over current donor blood screening system, where batch processing renders the prioritization of samples already in a queue or the introduction a sample to be prioritized over those already in the queue impossible. In certain embodiments, the random-access approach of the systems of the present disclosure provides for the rapid deconstruction of pooled samples should a pathogen or infectious agent be detected. For example, rather than waiting for the entire batch that included the positive pooled sample to be processed, the systems of the present disclosure allow for the prioritization of rescreening of sub-pools or individual donor samples to deconstruct the positive pooled sample, thereby substantially increasing overall efficiency of the donor blood screening process.

Additionally, or alternatively, and in accordance with another aspect of the disclosed subject matter, allowing for changes to the prioritization of samples after the samples have been loaded into the system, but before sample preparation has begun, or after sample preparation has completed, but before target amplification has begun, the random-access approach of the systems of the present disclosure can also allow for changes to the nucleic acid analysis applied to a sample or plurality of samples after preparation of the samples. For example, but not by way of limitation, because the systems of the present disclosure do not require batched processing, changes to the specific nucleic acid analysis implemented with respect to any particular sample can be modified prior the initiation of a nucleic acid analysis of the sample. In addition, because the methods and systems of the present disclosure can use a unified sample preparation process path, e.g., as illustrated in FIG. 17, changes to the nucleic acid analysis can, in certain embodiments, be made even after sample preparation is complete.

Additionally, or alternatively, the total number of nucleic acid analyses the systems of the present disclosure can perform can vary based on the system's ability to hold and access the nucleic acid analysis reagents necessary for carrying out sample preparation, amplification, and detection to determine the presence or absence in the sample of a particular pathogen or infectious agent. For example, in certain embodiments, the number of nucleic acid analyses available on a system of the present disclosure is 2 or more, 3 or more, 4 or more, 5 or more, 6 or more, 7 or more, 8 or more, 9 or more, 10 or more, 15 or more, 20 or more, 30 or more, 40 or more (e.g., 48 or more), or 50 or more nucleic acid analyses. In certain embodiments, the nucleic acid analyses can be performed in parallel.

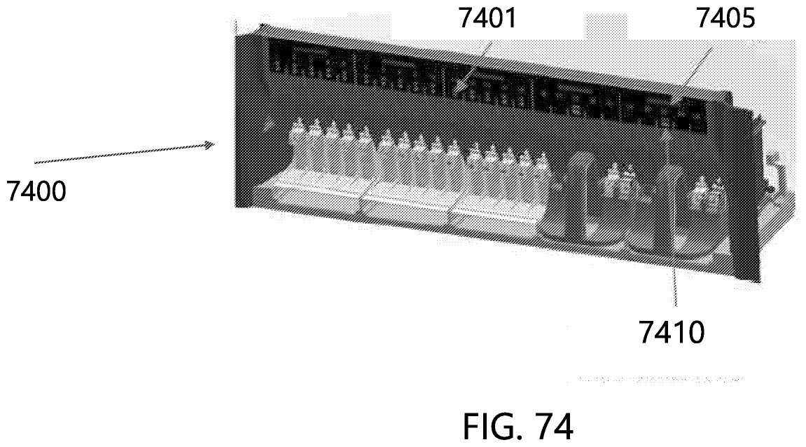

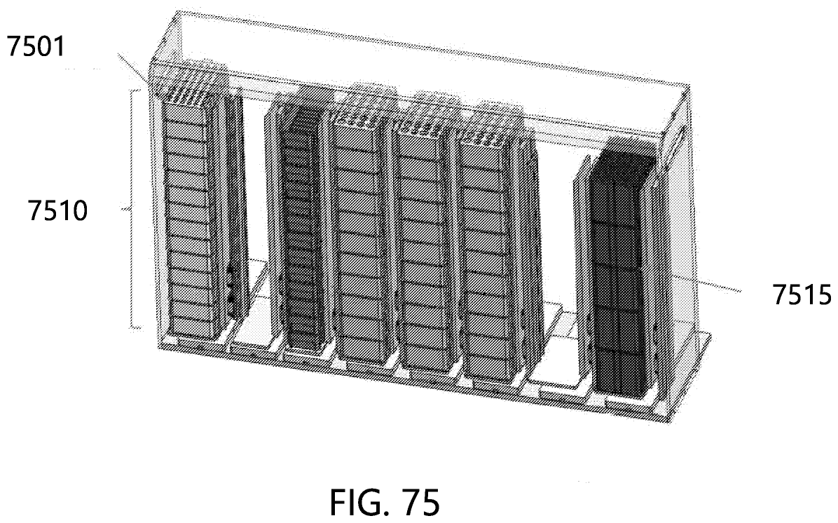

Additionally, or alternatively, the total number of nucleic acid analyses the systems of the present disclosure can perform can vary based on the system's ability to hold and access the nucleic acid analysis reagents necessary for carrying out sample preparation, amplification, and detection to determine the presence or absence in the sample of a particular pathogen or infectious agent. For purpose of illustration not limitation, FIG. 74 depicts an exemplary sample loading bay 7400 configured to allow batch and individual rack sample loading. The sample loading bay 7400 includes rack status LED 7401, priority light 7405, and tray status LED 7410. As embodied herein, the sample loading bay can load up to 360 samples and allow loading of different trays of samples for batch or individual loading. For purpose of illustration not limitation, FIG. 75 depicts an exemplary tray stack 7510 for use in the loading bay 7400. As embodied herein, the tray stack 7510 can include multiple trays 7501 with samples. Illustrated as 7515, the front of the trays can be accessible by the user directly.

### 6.4.2 Batched Sample Analysis Systems

Additionally, or alternatively, and in accordance with another aspect of the disclosed subject matter, the systems of the present disclosure can process samples and/or nucleic acid analyses in a “batch” format. As used herein, a batch format involves processing two or more samples and/or nucleic acid analyses in concert. The total number of batched samples and/or nucleic acid analyses can vary, and in certain aspects the number of batched samples and/or nucleic acid analyses is 2 or more, 5 or more, 10 or more, 15 or more, 20 or more, 30 or more, 40 or more (e.g., 48 or more), or 96 or more samples and/or nucleic acid analyses processed in concert.

### 6.4.3 Continuous Access Systems

Additionally, or alternatively, and in accordance with another aspect of the disclosed subject matter, the systems of the present disclosure, including systems capable of random access processing or batched processing, can include redundant components for sample processing and nucleic acid analysis, redundant loading/storage areas for, e.g., samples, reagents, sample processing cartridges, pipette tips, and/or the like. The redundant components enable the system to run (including presenting sample results/data) continuously and provide continuous operator access during the replenishment or removal of samples, bulk fluids, reagents, commodities (e.g., reaction vessels and reaction vessel caps, sample preparation (SP) cartridges, pipette tips and trays, assay plates, ancillary reagent packs, and/or the like), and waste, without ceasing operation of the system. By “continuous operator access” is meant an operator of the system can replenish and/or remove samples, bulk fluids, reagents, commodities, and waste without ceasing operation of the system, e.g., without interrupting any aspect of the sample preparation and analysis functions of the system. For purpose of illustration not limitation, the systems of the present disclosure can include a biohazard waste drawer, a reagent storage, a bulk solution drawer, a solid waste drawer, a consumable loader bay, a bulk solution reservoir and pump bay, a vacuum pump bay, a pipettor pump bay, and/or a liquid waste reservoir. An exemplary continuous operator access system to allow for continuous access and not requiring stopping the system to add samples and reagents to the system is disclosed in U.S. Pat. No. 9,335,338 and is herein incorporated by reference in its entirety.

### 6.4.4 Robotic Pipettors

Additionally, or alternatively, and in accordance with another aspect of the disclosed subject matter, e.g., as illustrated in FIG. 54B, the automated analysis systems 3100 of the present disclosure can include a robotic pipettor 3114. In certain embodiments, the robotic pipettor can access the system positions required for pipetting to accomplish sample preparation and processing, and RV capping and transport.

Additionally, or alternatively, the robotic pipettor 3114 can interact with, e.g., pipette tips at an internal location of the pipette tip loading area 3104; sample tubes at an internal location of the sample loading area 3102, ancillary reagents present at an internal location of the ancillary reagent loading area 3106, the sample preparation station 3110, assay reagents (e.g., lyophilized assay reagents present at an internal location of the assay reagent loading area 3108), a pipette tip and/or RV waste location, and the sample analysis station 3112. In certain embodiments, the pipettor 3114 can perform, e.g., transfer of samples and reagents to pretreatment or lysis wells; transfer pretreated samples from pretreatment wells to lysis wells; access eluate wells, auxiliary wells, and plunger disposal locations; access RV caps; fill RVs with eluate and reagents; access filled RVs; and access RV wells of the analysis station.

Additionally, or alternatively, e.g., as illustrated in FIG. 54C, a robotic pipettor of the present disclosure is movable in the X, Y and Z axes (e.g., via drive/servo motor assemblies) to interact with one or more of the system areas/stations described herein.

Additionally, or alternatively, e.g., as illustrated in FIG. 54D, the robotic pipettor 3300 has the same number of pipetting barrels 3302 as lanes of used in connection with the sample preparation strategy. For example, when a sample preparation system has 4 lanes (e.g., uses a 4-channel SP cartridge), the robotic pipettor 3300 can have 4 pipetting barrels 3302, such that the robotic pipettor 3300 can aspirate and/or dispense regents, samples, purified nucleic acids, and/or the like from corresponding wells of each lane/channel of an SP cartridge simultaneously.

Additionally, or alternatively, the robotic pipettor 3300 can include a camera for capturing images of the of the system for local analysis as well as remote analysis for purpose of maintenance, performance, automated correction, and the like. In some embodiments, the camera can also be used in reading barcodes present on assay plates and ancillary bottles. It can also be used to identify consumable characteristics, such as tip type.

Additionally, or alternatively, e.g., as illustrated in FIG. 54B, the robotic pipettor 3114 can pick up a disposable pipette tip from the pipette tip storage area 3104, aspirates a sample from a sample tube present at the sample introduction area 3102, and dispenses the sample into a first well. In certain aspects, the robotic pipettor 3114 can aspirate a purified sample from a second well and dispenses the purified sample into an assay reagent well (e.g., a well of an assay reagent plate). In certain embodiments, the assay reagent well can include lyophilized assay reagents. According to certain embodiments, the robotic pipettor 3114 can transfer a mixture that includes the purified sample and assay reagents from the assay reagent plate to a reaction vessel. According to certain embodiments, the robotic pipettor 3114 can pick up a reaction vessel cap and caps the reaction vessel. In certain aspects, the robotic pipettor 3114 can pick up the capped reaction vessel and transport the capped reaction vessel to a well of one of the sample analysis units of the sample analysis station 3112 described herein.

For purpose of example and explanation and not limitation, the use of disposable pipette tips with the robotic pipettor 3114 can provide advantages, such as for system throughput. For example and not limitation and as embodied herein, the use of disposable pipette tips can allow additional time for the moveable robotic pipettor to aspirate and dispense samples during the 24 second period between rotations of the sample preparation carousel. As embodied herein, the ability to perform additional aspiration and/or dispense operations between movement of the carousel can allow for additional sample preparation steps, such as sample pooling, while minimizing the impacts on overall system throughput. For purpose of example and illustration and not limitation, washing reusable pipette tips can take longer than changing disposable pipette tips on the moveable robotic pipettor. Additionally or alternatively, additional hardware, such as pipette tip washing hardware, can be omitted when disposable pipette tips are used.

Additionally, or alternatively, e.g., as illustrated in FIG. 54B, the robotic pipettor 3112 includes features that find use, e.g., in reducing or eliminating cross-contamination. For example, in certain aspects, the robotic pipettor has one or more (e.g., any combination) of the following features: an air-based pipetting mechanism; the ability to detect the level of a liquid in a container (e.g., the liquid level in a sample tube, reagent tube, or well, etc.); the ability to aspirate from an upper level (e.g., the top) of a liquid to prevent liquid drop contamination on the outside of the pipette tips; pipette tip material that discourages or prevents liquid from clinging to the outside of pipette tips; the formation (or “aspiration”) of an air gap to move aspirated liquid further up the pipette tip prior to movement, e.g., to prevent drips during movement (e.g., from a sample tube to a container into which the aspirated sample will be dispensed); one or more pressure sensors within the pipettor (e.g., one or more barrels of the pipettor) for sensing, e.g., fluid movement in the pipette tip (e.g., unanticipated fluid movement in the tip); a movement path such that the pipettor (e.g., with sample) never travels above an SP cartridge.

### 6.4.5 Pipette Tip Loading Areas

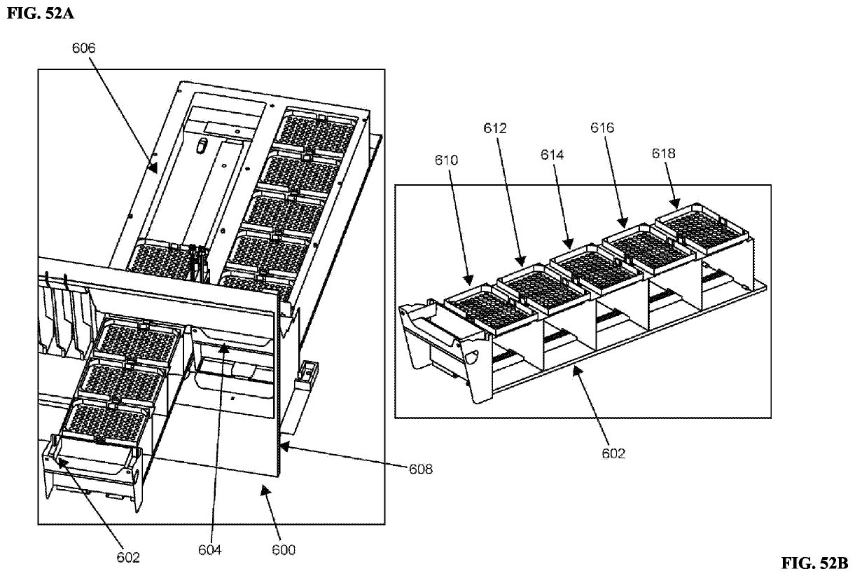

Additionally, or alternatively, and in accordance with another aspect of the disclosed subject matter, the automated sample preparation systems of the present disclosure can include a pipette tip loading area 600, e.g., as illustrated in FIG. 52A and FIG. 52B. In certain aspects, the pipette tip loading area 600 includes one or more (e.g., 2, 3, or 4) pipette tip drawers 602 and 604 each having positions for two or more pipette tip racks 610-618, to facilitate the loading of tip racks into the system and can comprise a front panel 608. According to certain embodiments, the one or more drawers can be completely removed from the system, e.g., as illustrated in FIG. 52B, to enable loading of tip racks 610-618 at a location remote from the system, e.g., at a different area within a facility.

Additionally, or alternatively, side-loading of tip racks can reduce the occurrence of tips being displaced from the tip racks upon loading of the tip racks into the drawers, which displacement occurs at an appreciable level when loading the tip racks from the top, e.g., due to inaccurate placement of tip racks into open positions in the drawer when loading from above.

### 6.4.6 Ancillary Reagent Loading Areas

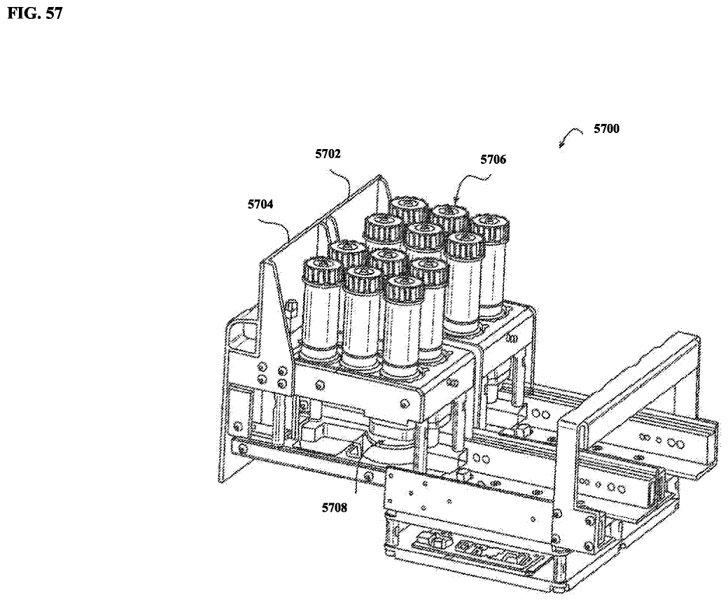

Additionally, or alternatively, and in accordance with another aspect of the disclosed subject matter, the automated sample preparation systems of the present disclosure can include one or more ancillary (or auxiliary) reagent loading area 5700, e.g., as illustrated in FIG. 57. Ancillary reagents can include, e.g., magnetic particles for capturing nucleic acids, elution buffer for eluting purified nucleic acids, and/or the like.

According to certain embodiments, the ancillary reagent loading area 5700 includes one or more drawers 5702 and 5704 into which individual ancillary reagent tubes 5706, or a pack of ganged ancillary reagent tubes, are loaded. Upon loading and closing of the one or more ancillary reagent loading drawers 5702 and 5704, the system can detect that it has been loaded with ancillary reagents.

In certain aspects, when one or more ancillary reagent tubes 5706 include magnetic particles, the ancillary reagent loading area 5700 includes a mixing motor 5708 to mix the magnetic particles, thereby maintaining the particles in suspension for consistent aspiration by a pipettor robot of the system. The mixing motor 5708 can work by alternatingly rotating the ancillary reagent tube 5706 in counterclockwise and clockwise directions.

According to certain embodiments, the operator loads ancillary reagent tubes 806 into the system in the form of ancillary reagent packs. The types of reagents within the tubes of the ancillary reagent packs can vary and can be selected by an operator of the system.

### 6.4.7 Liquid and Solid Waste Storage Areas

Additionally, or alternatively, and in accordance with another aspect of the disclosed subject matter, the automated analysis systems of the present disclosure include one or more liquid waste storage areas and/or one or more solid waste storage areas, including, but not limited to, where such storage areas are located in a drawer that also includes a bulk reagent storage area, e.g., drawer 5800 of FIG. 58. In certain aspects, a system of the present disclosure is adapted to dispose of liquid waste into one or more liquid waste containers at one or more liquid waste storage areas, where the liquid waste can be from, e.g., used reaction vessels, fill stations (e.g., overflow, wash, prime, and purge liquids), bulk reagent cradle overflow, and/or the like. According to certain embodiments, a system includes two liquid waste containers, e.g., two 2-liter liquid waste containers. That is, a liquid waste storage area can store the liquid waste more efficiently. Alternatively, or additionally, a solid waste robot can move a used tray in a horizontal or vertical direction to a trash bin area after the samples in the selected tray have been depleted. Additionally, or alternatively, the user can remove the used trays from the trash bin area. An exemplary bulk reagent system is disclosed in U.S. Pat. No. 9,823,263 and herein incorporated by reference in its entirety.

The system can include one or more solid waste storage areas. According to one embodiment, an analysis system of the present disclosure includes a solid waste storage area present in a drawer of the system, e.g., drawer 5800 of FIG. 58. In certain embodiments, one or more container, e.g., 5810 of FIG. 58, for disposing of capped reaction vessels (e.g., which have already undergone sample analysis), pipette tips, and/or the like, is present in a drawer, e.g., 5800 of FIG. 58. For example, it can be a drawer 5800 that also includes a bulk reagent storage area and a liquid waste storage area. The solid waste items can be released by the system (e.g., by the robotic pipettor of the system) above the upper opening of a chute (e.g., 5808 of FIG. 58) which is disposed above the container 5810. The drawer 5800 and chute 5808 can be configured such that the chute 5808 is only in communication with the container 5810 when the drawer 5800 is closed.

Specific mechanisms/configurations can be implemented for ensuring that the chute 5808 is only in communication with the container 5810 when the drawer 5800 is closed. For example, in certain aspects, a motor is operably connected to a chute flap. The motor is controlled by system electronics to position the chute flap such that the chute is in communication with the container 5810 when the drawer is closed and not in communication with the container when the drawer is open. According to some embodiments, when it is desirable to access the contents of the drawer 5800, a user of the system can request such access, e.g., via a user interface (e.g., button, touchscreen, and/or the like). Upon such a request, system electronics instruct the motor to position the chute flap such that the chute 5808 is no longer in communication with the container 5810. Optionally, when such a request is made, system electronics instruct a locking mechanism on the drawer 5800 to switch the drawer 5800 from a locked to unlocked configuration, e.g., subsequent to the chute flap handle being positioned such that the chute is no longer in communication with the container. An indicator (e.g., a light or particular color thereof) can be provided to the user of the system to indicate that the drawer can be safely opened.

In certain aspects, an analysis system of the present disclosure includes a solid waste storage area that includes one or more solid waste containers 5810 into which used reaction vessels are disposed. The reaction vessels can be disposed of by removal of the vessels from sample processing units of the sample processing station by a solid (or liquid/solid) waste handling module (e.g., liquid/solid waste handling module) which transports the reaction vessels to a position above one of the two containers, and drops the reaction vessels into the container 5810.

### 6.4.8 Bulk Fluid Storage Areas

Additionally, or alternatively, and in accordance with another aspect of the disclosed subject matter, the automated sample preparation systems of the present disclosure can include, e.g., as illustrated in FIG. 58, a bulk fluid (or “bulk reagent”) storage area (or “drawer”) 5800 including, in certain embodiments, a front cover 5802. Bulk reagents and are commonly used reagents that can be dispensed into a vessel, e.g., a wash vessel or lysis tube, with a pump and nozzle 5812 and do not require special manipulation, e.g., resuspension of the container's contents. In certain aspects, bulk fluids/reagents include sample lysis buffer, alcohol (e.g., ethanol), nucleic acid wash solutions, molecular grade water, vapor barrier reagent(s), and/or the like.

In certain aspects, bulk reagents are stored in bottles 5804 and 5806 in the bulk fluid storage area 5800. Bulk reagent bottles 5804 and 5806 can be sized to contain a desired volume of bulk reagents. For example, the bottles 5804 and 5806 can be sized to contain from 500 ml to 1.5 L (e.g., 1 L) of bulk fluid. For purpose of illustration not limitation, the container volume for lysis buffer can be around 2 L. The container volume for elution buffer can be around 650 mL. The container volume for Mg activator can be around 650 mL.

According to certain embodiments, one or more bulk reagents are provided in bottles that include a keyed cap. The keyed cap can include a keying element with at least one annular ring protrusion. The one or more annular rings provide a specific configuration of rings and spaces defined by the rings. The specific configuration created by the one or more annular rings functions as a “key” that requires a corresponding configuration on a receiving device in the bulk reagent storage area, to enable the keying element on the cap to be received. For example, the volume for Cu—Ti particles cartridge can be around 31.5 mL. The volume for PK cartridge can be around 11 mL. The volume for IC cartridge can be around 26.2 mL. For example, the corresponding keying element on the receiving device will be shaped and sized to properly align and receive the one or more annular rings of the keyed cap. For instance, the keying element on the receiving device can include one or more annular grooves or wells that are positioned appropriately to align with the one or more annular rings on the keyed cap. Furthermore, the keyed element on the receiving device can include one or more annular rings that are positioned appropriately to align with one more spaces on the keying element on the keyed cap that are defined by the one or more annular rings on the keyed cap. Further details regarding keyed caps useful for bulk reagent bottles and bulk reagent storage areas of the systems of the present disclosure can be found in U.S. Patent Application Publication No. 2014/0263316.

In certain embodiments, the bulk reagent storage area 5800 includes a fluidic interface 5812 that interfaces (e.g., via hoses) with a bulk fluid dispense station that dispenses bulk fluids into the appropriate wells of SP cartridges.

The bulk reagent storage area can include one or more reservoirs in fluid communication with one or more bulk reagent bottles 5804. For example, when a bulk reagent bottle 5804 is loaded into the bulk reagent storage area, the liquid contents of the bottle can empty into a reservoir, enabling an operator of the system to remove the bottle 5804 (and replace the bottle with a new (filled) reagent bottle 5806, if desired) at any convenient time during continuous operation of the system.

For purpose of example and not limitation, FIG. 68A and FIG. 68B illustrate an exemplary system including a bulk reagent storage area 6836. For purpose of example and as embodied herein, reagents, including reagents for sample preparation and nucleic acid amplification and detection can be stored and managed at the bulk reagent storage area 6836. For purpose of example and as embodied herein, reagents can be stored in containers at the bulk reagent storage area 6836. The type, size, and position of the container used for a respective reagent can be selected according to the desired performance of the system. Additionally or alternatively, and as embodied herein, some or all reagent containers can be configured for direct pluming and/or continuous filing. For purpose of example and as embodied herein, the bulk reagent storage area 6836 can include containers for lysis buffer, elution buffer, and Mg activator. As embodied herein, the lysis buffer, elution buffer, and Mg activator containers can be direct plumbed for continuous filing. Additionally or alternatively, one or more full reagent containers can be loaded into the bulk reagent storage area and replaced when the reagent in the container is depleted. The volume and configuration of each reagent container can be selected based on the desired performance of the system, including taking into account the volume of reagent used (e.g., for sample preparation). For purpose of example and as embodied herein, reagent storage area 6836 can include reagent containers for storing lysis buffer, elution buffer, Mg activator, CuTi particles, proteinase K (PK), internal control (IC), Oligos, and RPA master mix reagents as described above.

For purpose of example and as embodied herein, one or more robotic pipettors can be used to transfer reagents from the bulk reagent storage area 6836 to a desired location with the system 6800. For example and not limitation, and as embodied herein, system 6800 can include a sample preparation reagent pipettor 6817, which can be used to transfer reagents from the bulk reagent storage area 6836 to, for example, lysis carousel 6805. Additionally or alternatively, and as further embodied herein, system 6800 can include an amplification reagent pipettor 6815, which can, for example, be used to transfer reagents from the bulk reagent storage area 6836 to, for example, amplification and detection system 6807. Additionally or alternatively, and as further embodied herein, system 6800 can include a sample/reagent pipettor 6811, which can, for example, be used to transfer reagents from the bulk reagent storage area 6836 to, for example, wash track 6801. The position and configuration of the bulk reagent storage area 6836 and reagent containers within the bulk reagent storage area 6836 can be selected based on desired performance of the system. For purpose of example and as embodied herein, the bulk reagent storage area can be configured to facilitate reagent access by multiple robotic pipettors, such as for example sample preparation reagent pipettor 6817, amplification reagent pipettor 6815, and sample/reagent pipettor 6811. Additionally or alternatively, and as further embodied herein, the bulk reagent storage area 6836 can include one or more robots which can, for example, position reagents as desired such as for example to facilitate transfer of the reagents from the bulk reagent storage area by a robotic pipettor.

### 7. Additional Screening, Preparation and Processing Aspects

As described above, the high-throughput NAT (HTNAT) systems and methods described herein can be used in conjunction with additional screening and/or processing technologies. Non-limiting examples of additional screening and/or processing technologies include serological testing, alternative nucleic acid analyses and pathogen reduction technologies.

### 7.1 Serological Testing

In certain embodiments, the methods of screening a sample, e.g., screening a sample of blood for release of a donor material for clinical use, described herein can include HTNAT screening and serological testing to identify pathogens or infectious agents within the sample, e.g., sample of donor blood. For example, and but limitation, HTNAT and serological testing can be performed on individual or pooled samples, as described herein. For example, but not limitation, HTNAT can be performed on pooled samples and serological testing can be performed on individual samples. Additionally or alternatively, HTNAT can be performed on individual samples and serological testing can be performed on pooled samples. Additionally or alternatively, HTNAT and serological testing can both be performed on individual samples or pooled samples, respectively.

In certain embodiments, HTNAT can be performed to identify one or more pathogens or infectious agents in a sample, e.g., sample of donor blood, and serological testing can be performed to identify one or more different pathogens or infectious agents in the same sample, e.g., same sample of donor blood, or a second sample, e.g., a second sample of blood, from the same donor. For example, but not by way of limitation, HTNAT can be performed to identify one or more viruses in a sample, e.g., sample of donor blood, and serological testing can be performed to identify one or more bacteria in the same sample, e.g., same sample of donor blood, or a second sample, e.g., a second sample of blood, from the same donor.

### 7.2 Pathogen Reduction Technologies (PRT)

In certain embodiments, the methods of screening a sample, e.g., a blood sample, disclosed herein can be performed in combination with pathogen reduction technologies. For purpose of example and not limitation, after a nucleic acid analysis has been performed on a donor sample as described herein and a determination of the absence of a predetermined level of nucleic acids derived from pathogens or infectious agents has been made, a material from the donor or blood from the donor can be treated using pathogen reduction technologies.

Any pathogen reduction technology known in the art can be used with the disclosed subject matter. In certain embodiments, the pathogen reduction technology can include at least one photosensitizer. In certain embodiments, the pathogen reduction technology can be an agent that intercalates into nucleic acids present within the donor material. In certain embodiments, the pathogen reduction technology can be an agent that causes dimers in nucleic acids present within the donor material. For example, and not limitation, compounds such as amotosalen and/or riboflavin can be added to a donor material and the donor material can then be exposed to UV light, e.g., UVA or UVA/UVB, which can cause the amotosalen or riboflavin to bind to nucleic acids and prevent replication of pathogens. In certain embodiments, but not by way of limitation, the pathogen reduction technology can use amotosalen and UVA. In certain embodiments, the pathogen reduction technology can use riboflavin and UVA/UVB. In certain embodiments, the pathogen reduction technology can use amustaline and glutathione. In certain embodiments, the pathogen reduction technology can use methylene blue and visible light. Additionally or alternatively, pathogen reduction can include exposing donor material to UV light, e.g., UVC, without adding a photoactive compound to the donor material. In certain embodiments, pathogen reduction can include treating donor material with solvents and/or detergents, e.g., ethers, alcohols, volatile chlorinated hydrocarbons, acetone and/or chloroform.

Additional detail and explanation of pathogen reduction technologies is provided in Lu and Fung, Platelets treated with pathogen reduction technology: current status and future direction, F1000 Faculty Rev-40 (2020), the content of which is hereby incorporated in by reference in its entirety. Additional non-limiting examples of pathogen reduction technologies are disclosed in U.S. Pat. No. 6,969,367, the contents of which are hereby incorporated by reference herein in their entirety

Pathogen reduction can be performed on a broad range of donor materials. For example, but not by way of limitation, pathogen reduction can be used on whole blood, e.g., pooled whole donor blood or individual whole donor blood. In certain embodiments, the donor material can be plasma, red blood cells, platelets and/or a plasma-derived product. In certain embodiments, pathogen reduction can be used on plasma, e.g., pooled donor plasma or individual donor plasma. In certain embodiments, pathogen reduction can be used on a plasma-derived product including, but not limited to, coagulation factors, e.g., factor VIII, von Willebrand factor, and fibrinogen; protease inhibitors, e.g., alpha1-antitrypsin and C1-esterase inhibitor; albumin; and immunoglobulin G (IgG).

In certain embodiments, the method for screening a sample of donor blood for release of a donor material for clinical use can include performing a nucleic acid analysis on a first portion of the sample of donor blood to detect a pathogen or infectious agent in the sample of donor blood. In certain embodiments, if the results of the nucleic acid analysis indicate that a donor material can be released for clinical use, the donor material can be treated with a pathogen reduction technology. In certain embodiments, the determination that the donor material can be released for clinical use occurs in about 15 to about 45 minutes, e.g., about 20 to about 45 minutes, from aspiration of the sample of donor blood for performing the nucleic acid analysis. In certain embodiments, the donor material released for clinical use is treated with a pathogen reduction technology within about 1 minute to about 5 days of receiving a result from the nucleic acid analysis. In certain embodiments, how quickly the donor material can be treated with a pathogen reduction technology depends on the type of donor material being treated. For example, but not by way of limitation, the pathogen reduction technology can be performed immediately after a plasma-derived product is manufactured.

In certain embodiments, the nucleic acid analysis of donor blood samples can be performed in a decentralized location where the pathogen reduction technology can also be performed, e.g., performed at the same location, e.g., physical location, to allow immediate treatment of the released donor material with a pathogen reduction technology. Non-limiting examples of decentralized locations include doctor offices, Urgent Care facilities, Emergency Departments and sample collection sites.

In certain embodiments, the method can further include performing an immunoassay on a second portion of the sample of donor blood for detecting one or more pathogens or infectious agents in the sample of donor blood. For example, but not by way of limitation, an immunoassay is performed on a portion of the sample of donor blood prior to the nucleic acid analysis. Alternatively, the nucleic acid analysis is performed on a portion of the sample of donor blood prior to the immunoassay. In certain embodiments, the nucleic acid analysis is performed simultaneously with the immunoassay. In certain embodiments, if a method of the present disclosure includes treating with a pathogen reduction technology, the method does not include performing an immunoassay.

In certain embodiments, treating a donor material with a pathogen reduction technology can eliminate the need to screen for certain pathogens. For example, but not by way of limitation, a method of the present disclosure that includes the treatment with a pathogen reduction technology does not include the screening of Zika Virus, Dengue Virus, Chikungunya Virus, Babesia and/or Plasmodium falciparum (Malaria) in the sample being tested. In certain embodiments, a method of the present disclosure that includes the treatment with a pathogen reduction technology can be used to screen a sample, e.g., a blood sample, for the presence of one or more pathogens such as SARS-CoV-2 (COVID-19), coronaviruses, HIV-1, HIV-2, HBV, HCV, CMV, Epstein-Barr virus (EBV), human T-lymphotropic virus (HTLV) Parvo B19 Virus, HAV, syphilis, Chlamydia, Gonorrhea, Dengue, Chikungunya, WNV, HEV, Creutzfeldt-Jakob disease (vCJD) and a combination thereof. In certain embodiments, a method of the present disclosure that includes the treatment with a pathogen reduction technology can be used to screen a sample, e.g., a blood sample, for the presence of one or more pathogens such as HIV-1, HIV-2, HBV, HCV, CMV, Parvovirus B19, HAV, Chlamydia, Gonorrhea, WNV, HEV and a combination thereof.

In certain embodiments, treating a donor material with a pathogen reduction technology can reduce the risk that the donor material may include one or more pathogens or infectious agents, e.g., new or emerging pathogens or infectious diseases. For example, but not by way of limitation, the pathogen or infectious agent that can be inactivated or reduced by the pathogen reduction technology may be present in the sample at levels that are not detectable using a method disclosed herein. In certain embodiments, a pathogen reduction technology can be used in combination with a nucleic acid analysis that includes the pooling of two or more blood donor samples.

### 8. EXEMPLARY EMBODIMENTS

A. The present disclosure provides a method of screening a sample of donor blood for release of a donor material for clinical use, comprising: performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis is indicative of release of the donor material for clinical use, and wherein release of the donor material for clinical use occurs in about 15 to about 60 minutes from initial aspiration of the sample of donor blood for performance of the nucleic acid analysis.

A1. The method of A, wherein release of the donor material for clinical use occurs in about 20 to about 60 minutes from initial aspiration of the sample of donor blood for performance of the nucleic acid analysis.

A2. The method of A-A1, wherein release of the donor material for clinical use occurs in about 20 minutes to about 45 minutes from initial aspiration of the sample of donor blood for performance of the nucleic acid analysis.

A3. The method of A-A1, wherein the nucleic acid analysis comprises a nucleic acid amplification and detection process of about 1 minute to about 20 minutes in duration.

A4. The method of A-A3, wherein each determination of a predetermined level of nucleic acid from at least one of the plurality of pathogens or infectious agents has a time to result of about 20 to about 45 minutes.

A5. The method of A-A4, wherein the nucleic acid analysis is performed on an automated system, and wherein at least about 70 results are obtained per hour per m³ of a volume occupied by the automated system.

A6. The method of A-A5, wherein the nucleic acid analysis is performed on an automated system, and wherein at least about 140 results are obtained per hour per m² of a footprint of the automated system.

A7. The method of A-A6, wherein the determination of a level equal to or greater than the predetermined level indicates that the donor material, donor blood, biological sample, or plasma is not released for clinical use.

A8. The method of A-A6, wherein the determination of a level less than the predetermined level indicates that the donor material, donor blood, biological sample, or plasma is released for clinical use.

A9. The method of A-A6, wherein the plurality of pathogens or infectious agents are selected from the group consisting of HIV-1, HIV-2, HBV, HCV, Parvovirus B19, HAV, WNV, Zika Virus, Dengue Virus, Chikungunya Virus, Usutu Virus, Babesia, Malaria, and HEV.

A10. The method of A-A6, wherein the plurality of pathogens or infectious agents are HIV-1, HIV-2, HCV, and HBV.

A11. The method of A-A6, wherein the plurality of pathogens or infectious agents are HIV-1, HIV-2, HCV, HBV, and WNV.

A12. The method of A-A6, wherein the plurality of pathogens or infectious agents are Zika Virus and WNV.

A13. The method of A-A6, wherein the plurality of pathogens or infectious agents are Chikungunya Virus and Dengue Virus.

A14. The method of A-A6, wherein the plurality of pathogens or infectious agents are Zika Virus, WNV, Chikungunya Virus and Dengue Virus.

A15. The method of A-A6, wherein the plurality of pathogens or infectious agents are Babesia and Malaria.

A16. The method of A-A6, wherein the plurality of pathogens or infectious agents are Parvovirus B19 and HAV.

A17. The method of A-A6, wherein the plurality of pathogens or infectious agents are HIV-1, HIV-2, HCV, HBV, WNV, and Zika Virus.

A18. The method of A-A6, wherein the plurality of pathogens or infectious agents are HIV-1, HIV-2, HCV, HBV, WNV, Zika Virus, and Chikungunya Virus.

A19. The method of A-A6, wherein the plurality of pathogens or infectious agents are HIV-1, HIV-2, HCV, HBV, WNV, Zika Virus, Chikungunya Virus, and Dengue Virus.

A20. The method of A-A6, wherein the plurality of pathogens or infectious agents are HIV-1, HIV-2, HCV, HBV, WNV, Zika Virus, Chikungunya Virus, Dengue Virus, and Babesia.

A21. The method of A-A6, wherein the plurality of pathogens or infectious agents are HIV-1, HIV-2, HCV, HBV, WNV, Zika Virus, Chikungunya Virus, Dengue Virus, Babesia, and Malaria.

A22. The method of A-A6, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

A23. The method of A-A6, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

A24. The method of A-A6, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

A25. The method of A-A6, wherein the plurality of pathogens or infectious agents are HIV-1, HIV-2, HCV, HBV, and Parvovirus B19.

A26. The method of A-A6, wherein the plurality of pathogens or infectious agents are HIV-1, HIV-2, HCV, HBV, Parvovirus B19, and HAV.

A27. The method of A-A6, wherein the plurality of pathogens or infectious agents are HIV-1, HIV-2, HCV, HBV, and Babesia.

A28. The method of A-A6, wherein the plurality of pathogens or infectious agents are HIV-1, HIV-2, HCV, HBV, and HAV.

A29. The method of A-A6, wherein the plurality of pathogens or infectious agents are HIV-1, HIV-2, HCV, HBV, and HEV.

A30. The method of A-A6, wherein the plurality of pathogens or infectious agents are HIV-1, HIV-2, HCV, HBV, and Zika Virus.

A31. The method of A-A6, wherein the plurality of pathogens or infectious agents are HIV-1, HIV-2, HCV, HBV, Zika Virus, and Dengue Virus.

A32. The method of A-A6, wherein the plurality of pathogens or infectious agents are HIV-1, HIV-2, HCV, HBV, Zika Virus, Dengue Virus, and Chikungunya Virus.

A33. The method of A-A6, wherein the plurality of pathogens or infectious agents are HIV-1, HIV-2, HCV, HBV, Zika Virus, Dengue Virus, and WNV.

A34. The method of A-A6, wherein the plurality of pathogens or infectious agents are HIV-1, HIV-2, HCV, HBV, Zika Virus, WNV, and Chikungunya Virus.

A35. The method of A-A6, wherein the plurality of pathogens or infectious agents are HIV-1, HIV-2, HCV, HBV, Zika Virus, WNV, Dengue Virus, and Chikungunya Virus.

A36. The method of A-A6, wherein the plurality of pathogens or infectious agents are HIV-1, HIV-2, HCV, HBV, and Malaria.

A37. The method of A-A6, wherein the plurality of pathogens or infectious agents are HIV-1, HIV-2, HCV, HBV, Malaria, and Babesia.

A38. The method of A-A6, wherein the plurality of pathogens or infectious agents are HIV-1, HIV-2, HCV, HBV, and Dengue Virus.

A39. The method of A-A6, wherein the plurality of pathogens or infectious agents are HIV-1, HIV-2, HCV, HBV, Dengue Virus, and Chikungunya Virus.

A40. The method of A-A6, wherein the plurality of pathogens or infectious agents are HIV-1, HIV-2, HCV, HBV, Dengue Virus, WNV, and Chikungunya Virus.

A41. The method of A-A6, wherein the plurality of pathogens or infectious agents are HIV-1, HIV-2, HCV, HBV, and Chikungunya Virus.

A42. The method of A-A6, wherein the plurality of pathogens or infectious agents are HIV-1, HIV-2, HCV, HBV, Chikungunya Virus, and Zika Virus.

A43. The method of A-A6, wherein the plurality of pathogens or infectious agents are HIV-1, HIV-2, HCV, HBV, Chikungunya Virus, Zika Virus, and WNV.

A44. The method of A-A6, wherein the plurality of pathogens or infectious agents are HIV-1, HIV-2, HCV, HBV, WNV, and Dengue Virus.

A45. The method of A-A6, wherein the plurality of pathogens or infectious agents are HIV-1, HIV-2, HCV, HBV, Chikungunya Virus, Zika Virus, and WNV.

A46. The method of A-A6, wherein: the plurality of pathogens or infectious agents are HIV-1, HIV-2, HCV, and HBV; and the nucleic acid analysis comprises multiplex analysis of HIV-1 and HIV-2.

A47. The method of A-A6, wherein: the plurality of pathogens or infectious agents are HIV-1, HIV-2, HCV, and HBV; and the nucleic acid analysis comprises multiplex analysis of HIV-1, HIV-2, and HCV.

A48. The method of A-A6, wherein: the plurality of pathogens or infectious agents are HIV-1, HIV-2, HCV, and HBV; and the nucleic acid analysis comprises multiplex analysis of HIV-1, HIV-2, and HBV.

A49. The method of A-A6, wherein: the plurality of pathogens or infectious agents are HIV-1, HIV-2, HCV, and HBV; and the nucleic acid analysis comprises multiplex analysis of HCV, and HBV.

A50. The method of A-A6, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

A51. The method of A-A6, wherein: the plurality of pathogens or infectious agents are HIV-1, HIV-2, HCV, and HBV; and the nucleic acid analysis comprises multiplex analysis of HIV-1, HIV-2, HCV, and HBV.

A52. The method of A-A6, wherein: the plurality of pathogens or infectious agents comprise Zika Virus, WNV, Chikungunya Virus and Dengue Virus; the nucleic acid analysis comprises multiplex analysis of Zika Virus and WNV.

A53. The method of A-A6, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

A54. The method of A-A6, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

A55. The method of A-A6, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

A56. The method of A-A6, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

A57. The method of A-A6, wherein: the plurality of pathogens or infectious agents comprise Babesia and Malaria; and the nucleic acid analysis comprises multiplex analysis of Babesia and Malaria.

A58. The method of A-A6, wherein: the plurality of pathogens or infectious agents comprise Parvovirus B19 and HAV; and the nucleic acid analysis comprises multiplex analysis of Parvovirus B19 and HAV.

A59. The method of A-A6, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

A60. The method of A-A6, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

A61. The method of A-A6, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

A62. The method of A-A6, wherein the plurality of pathogens or infectious agents are Zika Virus and WNV; and wherein the predetermined levels are: at least 1-50 copies/mL of Zika Virus; and at least 1-50 copies/mL of WNV.

A63. The method of A-A6, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

A64. The method of A-A6, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

A65. The method of A-A6, wherein the plurality of pathogens or infectious agents are Babesia and Malaria; and wherein the predetermined levels are: at least 1-20 copies/mL of Babesia; and at least 1-50 copies/mL of Malaria.

A66. The method of A-A6, wherein the plurality of pathogens or infectious agents are Parvovirus B19 and HAV; and wherein the predetermined levels are: at least 1-40 IU/mL of Parvovirus B19; and at least 1-10 IU/mL of HAV.

A67. The method of A-A6, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

A68. The method of A-A6, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

A69. The method of A-A6, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

A70. The method of A-A6, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

A71. The method of A-A6, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

A72. The method of A-A6, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

A73. The method of A-A6, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

A74. The method of A-A6, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

A75. The method of A-A6, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

A76. The method of A-A6, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL ofParvovirus B19; and at least 1-10 IU/mL of HAV.

A77. The method of A-A6, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

A78. The method of A-A6, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

A79. The method of A-A6, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

A80. The method of A-A6, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

A81. The method of A-A6, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

A82. The method of A-A6, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

A83. The method of A-A6, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

A84. The method of A-A6, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

A85. The method of A-A6, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

A86. The method of A-A6, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

A87. The method of A-A6, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

A88. The method of A-A6, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

A89. The method of A-A6, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

A90. The method of A-A6, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

A91. The method of A-A6, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

A92. The method of A-A6, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

A93. The method of A-A6, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

A94. The method of A-A6, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

A95. The method of A-A6, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

A96. The method of A-A6, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

A97. The method of A-A6, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

A98. The method of A-A6, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

A99. The method of A-A6, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

A100. The method of A-A6, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

A101. The method of A-A6, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

A102. The method of A-A6, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

A103. The method of A-A6, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

A104. The method of A-A6, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

A105. The method of A-A6, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

A106. The method of A-A6, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

A107. The method of A-A6, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

A108. The method of A-A6, wherein the sample of donor blood is human donor blood.

A109. The method of A-A6, wherein the sample of donor blood is whole blood.

A110. The method of A-A6, wherein the sample of donor blood is lysed whole blood.

A111. The method of A-A6, wherein the sample of donor blood is serum.

A112. The method of A-A6, wherein the sample of donor blood is plasma.

A113. The method of A-A6 and A9-A112, wherein the release of the donor material is for transfusion.

A114. The method of A-A6 and A9-A112, wherein the release of the donor material is for use in a pharmaceutical.

A115. The method of A-A6 and A8-A112, wherein the release of the donor material is for use in a therapeutic treatment.

A116. The method of A-A6 and A9-A112, wherein the sample of donor blood is for use as a blood donation.

A117. The method of A-A6 and A9-A112, wherein the clinical use is an in vivo clinical use.

A118. The method of A-A6 and A9-A112, wherein the clinical use is an in vitro clinical use.

A118. The method of A-A6, A9-A112 and A117, wherein the clinical use is transfusion.

A119. The method of A-A6, A9-A112 and A117, wherein the clinical use is use in a pharmaceutical.

A120. The method of A-A6, A9-A112 and A117, wherein the clinical use is use in a therapeutic treatment.

A121. The method of A-A6, A9-A112 and A117, wherein the clinical use is organ or tissue donation.

A122. The method of A-A6, A9-A112 and A117, wherein the clinical use is use in a plasma-derived product.

A123. The method of A-A6, A9-A112 and A118, wherein the clinical use is use in disease diagnostics or in quality assurance/laboratory diagnostics.

A124. The method of A-A123, wherein the nucleic acid analysis comprises a nucleic acid amplification reaction.

A125. The method of A124, wherein the nucleic acid amplification reaction is an isothermal reaction.

A126. The method of A125, wherein the isothermal reaction is recombinase polymerase amplification.

A127. The method of A125, wherein the isothermal reaction is a nicking enzyme amplification reaction.

A128. The method of A-A127, wherein the nucleic acid analysis comprises optical detection of the presence of a nucleic acid derived from at least one of the plurality of pathogens or infectious agents.

A129. The method of A-A128, wherein the nucleic acid analysis comprises digital detection of the presence of a nucleic acid derived from at least one of the plurality of pathogens or infectious agents.

A130. The method of A-A129, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

A131. The method of A130, wherein the plurality of times comprises optically detecting the signal at a predetermined interval of about every 20 seconds or about every 30 seconds.

A132. The method of A130 and A131, wherein the optically detecting a fluorescent signal for a plurality of times occurs over a period of about 12 minutes.

B. The present disclosure provides a method of screening a sample of donor blood for release of a donor material for clinical use, comprising: performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis, is indicative of release of the donor material for clinical use, and wherein the method comprises screening a plurality of samples of donor blood for release of the donor material for clinical use and the determinations based on the nucleic acid analyses are performed within about 20 minutes to about 3.5 hours from initial aspiration of the first sample of donor blood for performance of the nucleic acid analysis.

B1. The method of B, wherein the determination of a level equal to or greater than the predetermined level indicates that the donor material is not released for clinical use.

B2. The method of B, wherein the determination of a level less than the predetermined level indicates that the donor material is released for clinical use.

B3. The method of B-B2, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

B4. The method of B, wherein the plurality of pathogens or infectious agents are HIV-1, HIV-2, HCV, and HBV.

B5. The method of B, wherein the plurality of pathogens or infectious agents are HIV-1, HIV-2, HCV, HBV, and WNV.

B6. The method of B, wherein the plurality of pathogens or infectious agents are Zika Virus and WNV.

B7. The method of B, wherein the plurality of pathogens or infectious agents are Chikungunya Virus and Dengue Virus.

B8. The method of B, wherein the plurality of pathogens or infectious agents are Zika Virus, WNV, Chikungunya Virus and Dengue Virus.

B9. The method of B, wherein the plurality of pathogens or infectious agents are Babesia and Malaria.

B10. The method of B, wherein the plurality of pathogens or infectious agents are Parvovirus B19 and HAV.

B11. The method of B, wherein the plurality of pathogens or infectious agents are HIV-1, HIV-2, HCV, HBV, WNV, and Zika Virus.

B12. The method of B, wherein the plurality of pathogens or infectious agents are HIV-1, HIV-2, HCV, HBV, WNV, Zika Virus, and Chikungunya Virus.

B13. The method of B, wherein the plurality of pathogens or infectious agents are HIV-1, HIV-2, HCV, HBV, WNV, Zika Virus, Chikungunya Virus, and Dengue Virus.

B14. The method of B, wherein the plurality of pathogens or infectious agents are HIV-1, HIV-2, HCV, HBV, WNV, Zika Virus, Chikungunya Virus, Dengue Virus, and Babesia.

B15. The method of B, wherein the plurality of pathogens or infectious agents are HIV-1, HIV-2, HCV, HBV, WNV, Zika Virus, Chikungunya Virus, Dengue Virus, Babesia, and Malaria.

B16. The method of B, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

B17. The method of B, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

B18. The method of B, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

B19. The method of B, wherein the plurality of pathogens or infectious agents are HIV-1, HIV-2, HCV, HBV, and Parvovirus B19.

B20. The method of B, wherein the plurality of pathogens or infectious agents are HIV-1, HIV-2, HCV, HBV, Parvovirus B19, and HAV.

B21. The method of B, wherein the plurality of pathogens or infectious agents are HIV-1, HIV-2, HCV, HBV, and Babesia.

B22. The method of B, wherein the plurality of pathogens or infectious agents are HIV-1, HIV-2, HCV, HBV, and HAV.

B23. The method of B, wherein the plurality of pathogens or infectious agents are HIV-1, HIV-2, HCV, HBV, and HEV.

B24. The method of B, wherein the plurality of pathogens or infectious agents are HIV-1, HIV-2, HCV, HBV, and Zika Virus.

B25. The method of B, wherein the plurality of pathogens or infectious agents are HIV-1, HIV-2, HCV, HBV, Zika Virus, and Dengue Virus.

B26. The method of B, wherein the plurality of pathogens or infectious agents are HIV-1, HIV-2, HCV, HBV, Zika Virus, Dengue Virus, and Chikungunya Virus.

B27. The method of B, wherein the plurality of pathogens or infectious agents are HIV-1, HIV-2, HCV, HBV, Zika Virus, Dengue Virus, and WNV.

B28. The method of B, wherein the plurality of pathogens or infectious agents are HIV-1, HIV-2, HCV, HBV, Zika Virus, WNV, and Chikungunya Virus.

B29. The method of B, wherein the plurality of pathogens or infectious agents are HIV-1, HIV-2, HCV, HBV, Zika Virus, WNV, Dengue Virus, and Chikungunya Virus.

B30. The method of B, wherein the plurality of pathogens or infectious agents are HIV-1, HIV-2, HCV, HBV, and Malaria.

B31. The method of B, wherein the plurality of pathogens or infectious agents are HIV-1, HIV-2, HCV, HBV, Malaria, and Babesia.

B32. The method of B, wherein the plurality of pathogens or infectious agents are HIV-1, HIV-2, HCV, HBV, and Dengue Virus.

B33. The method of B, wherein the plurality of pathogens or infectious agents are HIV-1, HIV-2, HCV, HBV, Dengue Virus, and Chikungunya Virus.

B34. The method of B, wherein the plurality of pathogens or infectious agents are HIV-1, HIV-2, HCV, HBV, Dengue Virus, WNV, and Chikungunya Virus.

B35. The method of B, wherein the plurality of pathogens or infectious agents are HIV-1, HIV-2, HCV, HBV, and Chikungunya Virus.

B36. The method of B, wherein the plurality of pathogens or infectious agents are HIV-1, HIV-2, HCV, HBV, Chikungunya Virus, and Zika Virus.

B37. The method of B, wherein the plurality of pathogens or infectious agents are HIV-1, HIV-2, HCV, HBV, Chikungunya Virus, Zika Virus, and WNV.

B38. The method of B, wherein the plurality of pathogens or infectious agents are HIV-1, HIV-2, HCV, HBV, WNV, and Dengue Virus.

B39. The method of B, wherein the plurality of pathogens or infectious agents are HIV-1, HIV-2, HCV, HBV, Chikungunya Virus, Zika Virus, and WNV.

B40. The method of B, wherein: the plurality of pathogens or infectious agents are HIV-1, HIV-2, HCV, and HBV; and the nucleic acid analysis comprises multiplex analysis of HIV-1 and HIV-2.

B41. The method of B, wherein: the plurality of pathogens or infectious agents are HIV-1, HIV-2, HCV, and HBV; and the nucleic acid analysis comprises multiplex analysis of HIV-1, HIV-2, and HCV.

B42. The method of B, wherein: the plurality of pathogens or infectious agents are HIV-1, HIV-2, HCV, and HBV; and the nucleic acid analysis comprises multiplex analysis of HIV-1, HIV-2, and HBV.

B43. The method of B, wherein: the plurality of pathogens or infectious agents are HIV-1, HIV-2, HCV, and HBV; and the nucleic acid analysis comprises multiplex analysis of HCV, and HBV.

B44. The method of B, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

B45. The method of B, wherein: the plurality of pathogens or infectious agents are HIV-1, HIV-2, HCV, and HBV; and the nucleic acid analysis comprises multiplex analysis of HIV-1, HIV-2, HCV, and HBV.

B46. The method of B, wherein: the plurality of pathogens or infectious agents comprise Zika Virus, WNV, Chikungunya Virus and Dengue Virus; the nucleic acid analysis comprises multiplex analysis of Zika Virus and WNV.

B47. The method of B, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

B48. The method of B, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

B49. The method of B, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

B50. The method of B, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

B51. The method of B, wherein: the plurality of pathogens or infectious agents comprise Babesia and Malaria; and the nucleic acid analysis comprises multiplex analysis of Babesia and Malaria.

B52. The method of B, wherein: the plurality of pathogens or infectious agents comprise Parvovirus B19 and HAV; and the nucleic acid analysis comprises multiplex analysis of Parvovirus B19 and HAV.

B53. The method of B, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

B54. The method of B, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

B55. The method of B, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

B56. The method of B, wherein the plurality of pathogens or infectious agents are Zika Virus and WNV; and wherein the predetermined levels are: at least 1-50 copies/mL of Zika Virus; and at least 1-50 copies/mL of WNV.

B57. The method of B, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

B58. The method of B, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

B59. The method of B, wherein the plurality of pathogens or infectious agents are Babesia and Malaria; and wherein the predetermined levels are: at least 1-20 copies/mL of Babesia; and at least 1-50 copies/mL of Malaria.

B60. The method of B, wherein the plurality of pathogens or infectious agents are Parvovirus B19 and HAV; and wherein the predetermined levels are: at least 1-40 IU/mL of Parvovirus B19; and at least 1-10 IU/mL of HAV.

B61. The method of B, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

B62. The method of B, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

B63. The method of B, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

B64. The method of B, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

B65. The method of B, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

B66. The method of B, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

B67. The method of B, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

B68. The method of B, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

B69. The method of B, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

B70. The method of B, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

B71. The method of B, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

B72. The method of B, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

B73. The method of B, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

B74. The method of B, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

B75. The method of B, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

B76. The method of B, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

B77. The method of B, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

B78. The method of B, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

B79. The method of B, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

B80. The method of B, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

B81. The method of B, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

B82. The method of B, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

B83. The method of B, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

B84. The method of B, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

B85. The method of B, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

B86. The method of B, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

B87. The method of B, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

B88. The method of B, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

B89. The method of B, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

B90. The method of B, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

B91. The method of B, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

B92. The method of B, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

B93. The method of B, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

B94. The method of B, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

B95. The method of B, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

B96. The method of B, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

B97. The method of B, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

B98. The method of B, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

B99. The method of B, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

B100. The method of B, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

B101. The method of B, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

B102. The method of any one of B-B101, wherein the sample of donor blood is human donor blood.

B103. The method of any one of B-B101, wherein the sample of donor blood is whole blood.

B104. The method of any one of B-B101, wherein the sample of donor blood is lysed whole blood.

B105. The method of any one of B-B101, wherein the sample of donor blood is serum.

B106. The method of any one of B-B101, wherein the sample of donor blood is plasma.

B107. The method of B and B3-B106, wherein the release of the donor material is for transfusion.

B108. The method of B and B3-B106, wherein the release of the donor material is for use in a pharmaceutical.

B109. The method of B and B3-B106, wherein the release of the donor material is for use in a therapeutic treatment.

B110. The method of B and B3-B106, wherein the sample of donor blood is for use as a blood donation.

B111. The method of B and B3-B106, wherein the clinical use is an in vivo clinical use.

B112. The method of B and B3-B106, wherein the clinical use is an in vitro clinical use.

B113. The method of B-B112, wherein the nucleic acid analysis comprises a nucleic acid amplification reaction.

B114. The method of B113, wherein the nucleic acid amplification reaction is an isothermal reaction.

B115. The method of B114, wherein the isothermal reaction is recombinase polymerase amplification.

B 116. The method of B114, wherein the isothermal reaction is a nicking enzyme amplification reaction.

B117. The method of B-B116, wherein the nucleic acid analysis comprises optical detection of the presence of a nucleic acid derived from at least one of the plurality of pathogens or infectious agents, e.g., at a plurality of times during the amplification reaction.

B118. The method of B-B116, wherein the nucleic acid analysis comprises digital detection of the presence of a nucleic acid derived from at least one of the plurality of pathogens or infectious agents.

B119. The method of B-B118, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

B120. The method of B117 or B119, wherein the plurality of times comprises optically detecting the signal at a predetermined interval of about every 20 seconds or about every 30 seconds.

B121. The method of B117, B119 and B120, wherein the optically detecting a fluorescent signal for a plurality of times occurs over a period of about 12 minutes.

C. The present disclosure provides a method of screening a sample of donor blood for release of a donor material for clinical use, comprising: performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis, is indicative of release of the donor material for clinical use, and wherein the nucleic acid analysis comprises a nucleic acid amplification and detection process of about 8 minutes to about 20 minutes in duration.

C1. The method of C, wherein the determination of a level equal to or greater than the predetermined level indicates that the donor material is not released for clinical use.

C2. The method of C, wherein the determination of a level less than the predetermined level indicates that the donor material is released for clinical use.

C3. The method of any one of C-C2, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

C4. The method of C, wherein the plurality of pathogens or infectious agents are HIV-1, HIV-2, HCV, and HBV.

C5. The method of C, wherein the plurality of pathogens or infectious agents are HIV-1, HIV-2, HCV, HBV, and WNV.

C6. The method of C, wherein the plurality of pathogens or infectious agents are Zika Virus and WNV.

C7. The method of C, wherein the plurality of pathogens or infectious agents are Chikungunya Virus and Dengue Virus.

C8. The method of C, wherein the plurality of pathogens or infectious agents are Zika Virus, WNV, Chikungunya Virus and Dengue Virus.

C9. The method of C, wherein the plurality of pathogens or infectious agents are Babesia and Malaria.

C10. The method of C, wherein the plurality of pathogens or infectious agents are Parvovirus B19 and HAV.

C11. The method of C, wherein the plurality of pathogens or infectious agents are HIV-1, HIV-2, HCV, HBV, WNV, and Zika Virus.

C12. The method of C, wherein the plurality of pathogens or infectious agents are HIV-1, HIV-2, HCV, HBV, WNV, Zika Virus, and Chikungunya Virus.

C13. The method of C, wherein the plurality of pathogens or infectious agents are HIV-1, HIV-2, HCV, HBV, WNV, Zika Virus, Chikungunya Virus, and Dengue Virus.

C14. The method of C, wherein the plurality of pathogens or infectious agents are HIV-1, HIV-2, HCV, HBV, WNV, Zika Virus, Chikungunya Virus, Dengue Virus, and Babesia.

C15. The method of C, wherein the plurality of pathogens or infectious agents are HIV-1, HIV-2, HCV, HBV, WNV, Zika Virus, Chikungunya Virus, Dengue Virus, Babesia, and Malaria.

C16. The method of C, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

C17. The method of C, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

C18. The method of C, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

C19. The method of C, wherein the plurality of pathogens or infectious agents are HIV-1, HIV-2, HCV, HBV, and Parvovirus B19.

C20. The method of C, wherein the plurality of pathogens or infectious agents are HIV-1, HIV-2, HCV, HBV, Parvovirus B19, and HAV.

C21. The method of C, wherein the plurality of pathogens or infectious agents are HIV-1, HIV-2, HCV, HBV, and Babesia.

C22. The method of C, wherein the plurality of pathogens or infectious agents are HIV-1, HIV-2, HCV, HBV, and HAV.

C23. The method of C, wherein the plurality of pathogens or infectious agents are HIV-1, HIV-2, HCV, HBV, and HEV.

C24. The method of C, wherein the plurality of pathogens or infectious agents are HIV-1, HIV-2, HCV, HBV, and Zika Virus.

C25. The method of C, wherein the plurality of pathogens or infectious agents are HIV-1, HIV-2, HCV, HBV, Zika Virus, and Dengue Virus.

C26. The method of C, wherein the plurality of pathogens or infectious agents are HIV-1, HIV-2, HCV, HBV, Zika Virus, Dengue Virus, and Chikungunya Virus.

C27. The method of C, wherein the plurality of pathogens or infectious agents are HIV-1, HIV-2, HCV, HBV, Zika Virus, Dengue Virus, and WNV.

C28. The method of C, wherein the plurality of pathogens or infectious agents are HIV-1, HIV-2, HCV, HBV, Zika Virus, WNV, and Chikungunya Virus.

C29. The method of C, wherein the plurality of pathogens or infectious agents are HIV-1, HIV-2, HCV, HBV, Zika Virus, WNV, Dengue Virus, and Chikungunya Virus.

C30. The method of C, wherein the plurality of pathogens or infectious agents are HIV-1, HIV-2, HCV, HBV, and Malaria.

C31. The method of C, wherein the plurality of pathogens or infectious agents are HIV-1, HIV-2, HCV, HBV, Malaria, and Babesia.

C32. The method of C, wherein the plurality of pathogens or infectious agents are HIV-1, HIV-2, HCV, HBV, and Dengue Virus.

C33. The method of C, wherein the plurality of pathogens or infectious agents are HIV-1, HIV-2, HCV, HBV, Dengue Virus, and Chikungunya Virus.

C34. The method of C, wherein the plurality of pathogens or infectious agents are HIV-1, HIV-2, HCV, HBV, Dengue Virus, WNV, and Chikungunya Virus.

C35. The method of C, wherein the plurality of pathogens or infectious agents are HIV-1, HIV-2, HCV, HBV, and Chikungunya Virus.

C36. The method of C, wherein the plurality of pathogens or infectious agents are HIV-1, HIV-2, HCV, HBV, Chikungunya Virus, and Zika Virus.

C37. The method of C, wherein the plurality of pathogens or infectious agents are HIV-1, HIV-2, HCV, HBV, Chikungunya Virus, Zika Virus, and WNV.

C38. The method of C, wherein the plurality of pathogens or infectious agents are HIV-1, HIV-2, HCV, HBV, WNV, and Dengue Virus.

C39. The method of C, wherein the plurality of pathogens or infectious agents are HIV-1, HIV-2, HCV, HBV, Chikungunya Virus, Zika Virus, and WNV.

C40. The method of C, wherein: the plurality of pathogens or infectious agents are HIV-1, HIV-2, HCV, and HBV; and the nucleic acid analysis comprises multiplex analysis of HIV-1 and HIV-2.

C41. The method of C, wherein: the plurality of pathogens or infectious agents are HIV-1, HIV-2, HCV, and HBV; and the nucleic acid analysis comprises multiplex analysis of HIV-1, HIV-2, and HCV.

C42. The method of C, wherein: the plurality of pathogens or infectious agents are HIV-1, HIV-2, HCV, and HBV; and the nucleic acid analysis comprises multiplex analysis of HIV-1, HIV-2, and HBV.

C43. The method of C, wherein: the plurality of pathogens or infectious agents are HIV-1, HIV-2, HCV, and HBV; and the nucleic acid analysis comprises multiplex analysis of HCV, and HBV.

C44. The method of C, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

C45. The method of C, wherein: the plurality of pathogens or infectious agents are HIV-1, HIV-2, HCV, and HBV; and the nucleic acid analysis comprises multiplex analysis of HIV-1, HIV-2, HCV, and HBV.

C46. The method of C, wherein: the plurality of pathogens or infectious agents comprise Zika Virus, WNV, Chikungunya Virus and Dengue Virus; the nucleic acid analysis comprises multiplex analysis of Zika Virus and WNV.

C47. The method of C, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

C48. The method of C, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

C49. The method of C, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

C50. The method of C, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

C51. The method of C, wherein: the plurality of pathogens or infectious agents comprise Babesia and Malaria; and the nucleic acid analysis comprises multiplex analysis of Babesia and Malaria.

C52. The method of C, wherein: the plurality of pathogens or infectious agents comprise Parvovirus B19 and HAV; and the nucleic acid analysis comprises multiplex analysis of Parvovirus B19 and HAV.

C53. The method of C, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

C54. The method of C, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

C55. The method of C, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

C56. The method of C, wherein the plurality of pathogens or infectious agents are Zika Virus and WNV; and wherein the predetermined levels are: at least 1-50 copies/mL of Zika Virus; and at least 1-50 copies/mL of WNV.

C57. The method of C, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

C58. The method of C, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

C59. The method of C, wherein the plurality of pathogens or infectious agents are Babesia and Malaria; and wherein the predetermined levels are: at least 1-20 copies/mL of Babesia; and at least 1-50 copies/mL of Malaria.

C60. The method of C, wherein the plurality of pathogens or infectious agents are Parvovirus B19 and HAV; and wherein the predetermined levels are: at least 1-40 IU/mL of Parvovirus B19; and at least 1-10 IU/mL of HAV.

C61. The method of C, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

C62. The method of C, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

C63. The method of C, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

C64. The method of C, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

C65. The method of C, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

C66. The method of C, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

C67. The method of C, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

C68. The method of C, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

C69. The method of C, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

C70. The method of C, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

C71. The method of C, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

C72. The method of C, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

C73. The method of C, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

C74. The method of C, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

C75. The method of C, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

C76. The method of C, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

C77. The method of C, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

C78. The method of C, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

C79. The method of C, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

C80. The method of C, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

C81. The method of C, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

C82. The method of C, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

C83. The method of C, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

C84. The method of C, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

C85. The method of C, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

C86. The method of C, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

C87. The method of C, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

C88. The method of C, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

C89. The method of C, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

C90. The method of C, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

C91. The method of C, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

C92. The method of C, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

C93. The method of C, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

C94. The method of C, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

C95. The method of C, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

C96. The method of C, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

C97. The method of C, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

C98. The method of C, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

C99. The method of C, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

C100. The method of C, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

C101. The method of C, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

C102. The method of C, wherein the sample of donor blood is human donor blood.

C103. The method of C, wherein the sample of donor blood is whole blood.

C104. The method of C, wherein the sample of donor blood is lysed whole blood.

C105. The method of C, wherein the sample of donor blood is serum.

C106. The method of C, wherein the sample of donor blood is plasma.

C107. The method of C and C2-C106, wherein the release of the donor material is for transfusion.

C108. The method of C and C3-C106, wherein the release of the donor material is for use in a pharmaceutical.

C109. The method of C and C2-C106, wherein the release of the donor material is for use in a therapeutic treatment.

C110. The method of C and C2-C106, wherein the sample of donor blood is for use as a blood donation.

C111. The method of C and C2-C106, wherein the clinical use is an in vivo clinical use.

C112. The method of C and C2-C106, wherein the clinical use is an in vitro clinical use.

C113. The method of C-C112, wherein the nucleic acid analysis comprises a nucleic acid amplification reaction.

C114. The method of C114, wherein the nucleic acid amplification reaction is an isothermal reaction.

C115. The method of C114, wherein the isothermal reaction is recombinase polymerase amplification.

C116. The method of C114, wherein the isothermal reaction is a nicking enzyme amplification reaction.

C117. The method of C-C116, wherein the nucleic acid analysis comprises optical detection of the presence of a nucleic acid derived from at least one of the plurality of pathogens or infectious agents.

C118. The method of C-C116, wherein the nucleic acid analysis comprises digital detection of the presence of a nucleic acid derived from at least one of the plurality of pathogens or infectious agents, e.g., at a plurality of times during the amplification reaction.

C119. The method of C-C118, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

C120. The method of C118 or C119, wherein the plurality of times comprises optically detecting the signal at a predetermined interval of about every 20 seconds or about every 30 seconds.

C121. The method of C118, C119 and C120, wherein the optically detecting a fluorescent signal for a plurality of times occurs over a period of about 12 minutes.

D. The present disclosure provides a method of screening a sample of donor blood for release of a donor material for clinical use, comprising: performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis, is indicative of release of the donor material for clinical use, and wherein each determination of a predetermined level of nucleic acid from at least one of the plurality of pathogens or infectious agents is completed in about 20 to about 45 minutes from initial aspiration of the sample of donor blood for performance of the nucleic acid analysis.

D1. The method of D, wherein the determination of a level equal to or greater than the predetermined level indicates that the donor material is not released for clinical use.

D2. The method of D, wherein the determination of a level less than the predetermined level indicates that the donor material is released for clinical use.

D3. The method of D, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

D4. The method of D, wherein the plurality of pathogens or infectious agents are HIV-1, HIV-2, HCV, and HBV.

D5. The method of D, wherein the plurality of pathogens or infectious agents are HIV-1, HIV-2, HCV, HBV, and WNV.

D6. The method of D, wherein the plurality of pathogens or infectious agents are Zika Virus and WNV.

D7. The method of D, wherein the plurality of pathogens or infectious agents are Chikungunya Virus and Dengue Virus.

D8. The method of D, wherein the plurality of pathogens or infectious agents are Zika Virus, WNV, Chikungunya Virus and Dengue Virus.

D9. The method of D, wherein the plurality of pathogens or infectious agents are Babesia and Malaria.

D10. The method of D, wherein the plurality of pathogens or infectious agents are Parvovirus B19 and HAV.

D11. The method of D, wherein the plurality of pathogens or infectious agents are HIV-1, HIV-2, HCV, HBV, WNV, and Zika Virus.

D12. The method of D, wherein the plurality of pathogens or infectious agents are HIV-1, HIV-2, HCV, HBV, WNV, Zika Virus, and Chikungunya Virus.

D13. The method of D, wherein the plurality of pathogens or infectious agents are HIV-1, HIV-2, HCV, HBV, WNV, Zika Virus, Chikungunya Virus, and Dengue Virus.

D14. The method of D, wherein the plurality of pathogens or infectious agents are HIV-1, HIV-2, HCV, HBV, WNV, Zika Virus, Chikungunya Virus, Dengue Virus, and Babesia.

D15. The method of D, wherein the plurality of pathogens or infectious agents are HIV-1, HIV-2, HCV, HBV, WNV, Zika Virus, Chikungunya Virus, Dengue Virus, Babesia, and Malaria.

D16. The method of D, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

D17. The method of D, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

D18. The method of D, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

D19. The method of D, wherein the plurality of pathogens or infectious agents are HIV-1, HIV-2, HCV, HBV, and Parvovirus B19.

D20. The method of D, wherein the plurality of pathogens or infectious agents are HIV-1, HIV-2, HCV, HBV, Parvovirus B19, and HAV.

D21. The method of D, wherein the plurality of pathogens or infectious agents are HIV-1, HIV-2, HCV, HBV, and Babesia.

D22. The method of D, wherein the plurality of pathogens or infectious agents are HIV-1, HIV-2, HCV, HBV, and HAV.

D23. The method of D, wherein the plurality of pathogens or infectious agents are HIV-1, HIV-2, HCV, HBV, and HEV.

D24. The method of D, wherein the plurality of pathogens or infectious agents are HIV-1, HIV-2, HCV, HBV, and Zika Virus.

D25. The method of D, wherein the plurality of pathogens or infectious agents are HIV-1, HIV-2, HCV, HBV, Zika Virus, and Dengue Virus.

D26. The method of D, wherein the plurality of pathogens or infectious agents are HIV-1, HIV-2, HCV, HBV, Zika Virus, Dengue Virus, and Chikungunya Virus.

D27. The method of D, wherein the plurality of pathogens or infectious agents are HIV-1, HIV-2, HCV, HBV, Zika Virus, Dengue Virus, and WNV.

D28. The method of D, wherein the plurality of pathogens or infectious agents are HIV-1, HIV-2, HCV, HBV, Zika Virus, WNV, and Chikungunya Virus.

D29. The method of D, wherein the plurality of pathogens or infectious agents are HIV-1, HIV-2, HCV, HBV, Zika Virus, WNV, Dengue Virus, and Chikungunya Virus.

D30. The method of D, wherein the plurality of pathogens or infectious agents are HIV-1, HIV-2, HCV, HBV, and Malaria.

D31. The method of D, wherein the plurality of pathogens or infectious agents are HIV-1, HIV-2, HCV, HBV, Malaria, and Babesia.

D32. The method of D, wherein the plurality of pathogens or infectious agents are HIV-1, HIV-2, HCV, HBV, and Dengue Virus.

D33. The method of D, wherein the plurality of pathogens or infectious agents are HIV-1, HIV-2, HCV, HBV, Dengue Virus, and Chikungunya Virus.

D34. The method of D, wherein the plurality of pathogens or infectious agents are HIV-1, HIV-2, HCV, HBV, Dengue Virus, WNV, and Chikungunya Virus.

D35. The method of D, wherein the plurality of pathogens or infectious agents are HIV-1, HIV-2, HCV, HBV, and Chikungunya Virus.

D36. The method of D, wherein the plurality of pathogens or infectious agents are HIV-1, HIV-2, HCV, HBV, Chikungunya Virus, and Zika Virus.

D37. The method of D, wherein the plurality of pathogens or infectious agents are HIV-1, HIV-2, HCV, HBV, Chikungunya Virus, Zika Virus, and WNV.

D38. The method of D, wherein the plurality of pathogens or infectious agents are HIV-1, HIV-2, HCV, HBV, WNV, and Dengue Virus.

D39. The method of D, wherein the plurality of pathogens or infectious agents are HIV-1, HIV-2, HCV, HBV, Chikungunya Virus, Zika Virus, and WNV.

D40. The method of D, wherein: the plurality of pathogens or infectious agents are HIV-1, HIV-2, HCV, and HBV; and the nucleic acid analysis comprises multiplex analysis of HIV-1 and HIV-2.

D41. The method of D, wherein: the plurality of pathogens or infectious agents are HIV-1, HIV-2, HCV, and HBV; and the nucleic acid analysis comprises multiplex analysis of HIV-1, HIV-2, and HCV.

D42. The method of D, wherein: the plurality of pathogens or infectious agents are HIV-1, HIV-2, HCV, and HBV; and the nucleic acid analysis comprises multiplex analysis of HIV-1, HIV-2, and HBV.

D43. The method of D, wherein: the plurality of pathogens or infectious agents are HIV-1, HIV-2, HCV, and HBV; and the nucleic acid analysis comprises multiplex analysis of HCV, and HBV.

D44. The method of D, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

D45. The method of D, wherein: the plurality of pathogens or infectious agents are HIV-1, HIV-2, HCV, and HBV; and the nucleic acid analysis comprises multiplex analysis of HIV-1, HIV-2, HCV, and HBV.

D46. The method of D, wherein: the plurality of pathogens or infectious agents comprise Zika Virus, WNV, Chikungunya Virus and Dengue Virus; the nucleic acid analysis comprises multiplex analysis of Zika Virus and WNV.

D47. The method of D, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

D48. The method of D, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

D49. The method of D, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

D50. The method of D, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

D51. The method of D, wherein: the plurality of pathogens or infectious agents comprise Babesia and Malaria; and the nucleic acid analysis comprises multiplex analysis of Babesia and Malaria.

D52. The method of D, wherein: the plurality of pathogens or infectious agents comprise Parvovirus B19 and HAV; and the nucleic acid analysis comprises multiplex analysis of Parvovirus B19 and HAV.

D53. The method of D, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

D54. The method of D, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

D55. The method of D, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

D56. The method of D, wherein the plurality of pathogens or infectious agents are Zika Virus and WNV; and wherein the predetermined levels are: at least 1-50 copies/mL of Zika Virus; and at least 1-50 copies/mL of WNV.

D57. The method of D, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

D58. The method of D, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

D59. The method of D, wherein the plurality of pathogens or infectious agents are Babesia and Malaria; and wherein the predetermined levels are: at least 1-20 copies/mL of Babesia; and at least 1-50 copies/mL of Malaria.

D60. The method of D, wherein the plurality of pathogens or infectious agents are Parvovirus B19 and HAV; and wherein the predetermined levels are: at least 1-40 IU/mL of Parvovirus B19; and at least 1-10 IU/mL of HAV.

D61. The method of D, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

D62. The method of D, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

D63. The method of D, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

D64. The method of D, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

D65. The method of D, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

D66. The method of D, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

D67. The method of D, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

D68. The method of D, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

D69. The method of D, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

D70. The method of D, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

D71. The method of D, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

D72. The method of D, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

D73. The method of D, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

D74. The method of D, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

D75. The method of D, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

D76. The method of D, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

D77. The method of D, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

D78. The method of D, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

D79. The method of D, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

D80. The method of D, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

D81. The method of D, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

D82. The method of D, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

D83. The method of D, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

D84. The method of D, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

D85. The method of D, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

D86. The method of D, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

D87. The method of D, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

D88. The method of D, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

D89. The method of D, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

D90. The method of D, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

D91. The method of D, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

D92. The method of D, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

D93. The method of D, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

D94. The method of D, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

D95. The method of D, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

D96. The method of D, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

D97. The method of D, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

D98. The method of D, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

D99. The method of D, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

D100. The method of D, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

D101. The method of D, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

D102. The method of any one of D-D101, wherein the sample of donor blood is human donor blood.

D103. The method of any one of D-D101, wherein the sample of donor blood is whole blood.

D104. The method of any one of D-D101, wherein the sample of donor blood is lysed whole blood.

D105. The method of any one of D-D101, wherein the sample of donor blood is serum.

D106. The method of any one of D-D101, wherein the sample of donor blood is plasma.

D107. The method of D and D2-D106, wherein the release of the donor material is for transfusion.

D108. The method of D and D2-D106, wherein the release of the donor material is for use in a pharmaceutical.

D109. The method of D and D2-D106, wherein the release of the donor material is for use in a therapeutic treatment.

D110. The method of D and D2-D106, wherein the sample of donor blood is for use as a blood donation.

D111. The method of D and D2-D106, wherein the clinical use is an in vivo clinical use.

D112. The method of D and D2-D106, wherein the clinical use is an in vitro clinical use.

D113. The method of D-D118, wherein the nucleic acid analysis comprises a nucleic acid amplification reaction.

D114. The method of D119, wherein the nucleic acid amplification reaction is an isothermal reaction.

D115. The method of D120, wherein the isothermal reaction is recombinase polymerase amplification.

D116. The method of D120, wherein the isothermal reaction is anicking enzyme amplification reaction.

D117. The method of D-D116, wherein the nucleic acid analysis comprises optical detection of the presence of a nucleic acid derived from at least one of the plurality of pathogens or infectious agents, e.g., at a plurality of times during the amplification reaction.

D118. The method of D-D116, wherein the nucleic acid analysis comprises digital detection of the presence of a nucleic acid derived from at least one of the plurality of pathogens or infectious agents.

D119. The method of D-D118, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

D120. The method of D117 or D119, wherein the plurality of times comprises optically detecting the signal at a predetermined interval of about every 20 seconds or about every 30 seconds.

D127. The method of D117, D119 or D119, wherein the optically detecting a fluorescent signal for a plurality of times occurs over a period of about 12 minutes.

E. The present disclosure provides a method of screening a sample of donor blood for release of a donor material for clinical use, comprising: performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis, is indicative of release of the donor material for clinical use, and wherein each determination of a predetermined level of nucleic acid from at least one of the plurality of pathogens or infectious agents has a time to result of about 20 to about 45 minutes.

E1. The method of E, wherein the determination of a level equal to or greater than the predetermined level indicates that the donor material is not released for clinical use.

E2. The method of E, wherein the determination of a level less than the predetermined level indicates that the donor material is released for clinical use.

E3. The method of E, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

E4. The method of E, wherein the plurality of pathogens or infectious agents are HIV-1, HIV-2, HCV, and HBV.

E5. The method of E, wherein the plurality of pathogens or infectious agents are HIV-1, HIV-2, HCV, HBV, and WNV.

E6. The method of E, wherein the plurality of pathogens or infectious agents are Zika Virus and WNV.

E7. The method of E, wherein the plurality of pathogens or infectious agents are Chikungunya Virus and Dengue Virus.

E8. The method of E, wherein the plurality of pathogens or infectious agents are Zika Virus, WNV, Chikungunya Virus and Dengue Virus.

E9. The method of E, wherein the plurality of pathogens or infectious agents are Babesia and Malaria.

E10. The method of E, wherein the plurality of pathogens or infectious agents are Parvovirus B19 and HAV.

E11. The method of E, wherein the plurality of pathogens or infectious agents are HIV-1, HIV-2, HCV, HBV, WNV, and Zika Virus.

E12. The method of E, wherein the plurality of pathogens or infectious agents are HIV-1, HIV-2, HCV, HBV, WNV, Zika Virus, and Chikungunya Virus.

E13. The method of E, wherein the plurality of pathogens or infectious agents are HIV-1, HIV-2, HCV, HBV, WNV, Zika Virus, Chikungunya Virus, and Dengue Virus.

E14. The method of E, wherein the plurality of pathogens or infectious agents are HIV-1, HIV-2, HCV, HBV, WNV, Zika Virus, Chikungunya Virus, Dengue Virus, and Babesia.

E15. The method of E, wherein the plurality of pathogens or infectious agents are HIV-1, HIV-2, HCV, HBV, WNV, Zika Virus, Chikungunya Virus, Dengue Virus, Babesia, and Malaria.

E16. The method of E, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

E17. The method of E, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

E18. The method of E, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

E19. The method of E, wherein the plurality of pathogens or infectious agents are HIV-1, HIV-2, HCV, HBV, and Parvovirus B19.

E20. The method of E, wherein the plurality of pathogens or infectious agents are HIV-1, HIV-2, HCV, HBV, Parvovirus B19, and HAV.

E21. The method of E, wherein the plurality of pathogens or infectious agents are HIV-1, HIV-2, HCV, HBV, and Babesia.

E22. The method of E, wherein the plurality of pathogens or infectious agents are HIV-1, HIV-2, HCV, HBV, and HAV.

E23. The method of E, wherein the plurality of pathogens or infectious agents are HIV-1, HIV-2, HCV, HBV, and HEV.

E24. The method of E, wherein the plurality of pathogens or infectious agents are HIV-1, HIV-2, HCV, HBV, and Zika Virus.

E25. The method of E, wherein the plurality of pathogens or infectious agents are HIV-1, HIV-2, HCV, HBV, Zika Virus, and Dengue Virus.

E26. The method of E, wherein the plurality of pathogens or infectious agents are HIV-1, HIV-2, HCV, HBV, Zika Virus, Dengue Virus, and Chikungunya Virus.

E27. The method of E, wherein the plurality of pathogens or infectious agents are HIV-1, HIV-2, HCV, HBV, Zika Virus, Dengue Virus, and WNV.

E28. The method of E, wherein the plurality of pathogens or infectious agents are HIV-1, HIV-2, HCV, HBV, Zika Virus, WNV, and Chikungunya Virus.

E29. The method of E, wherein the plurality of pathogens or infectious agents are HIV-1, HIV-2, HCV, HBV, Zika Virus, WNV, Dengue Virus, and Chikungunya Virus.

E30. The method of E, wherein the plurality of pathogens or infectious agents are HIV-1, HIV-2, HCV, HBV, and Malaria.

E31. The method of E, wherein the plurality of pathogens or infectious agents are HIV-1, HIV-2, HCV, HBV, Malaria, and Babesia.

E32. The method of E, wherein the plurality of pathogens or infectious agents are HIV-1, HIV-2, HCV, HBV, and Dengue Virus.

E33. The method of E, wherein the plurality of pathogens or infectious agents are HIV-1, HIV-2, HCV, HBV, Dengue Virus, and Chikungunya Virus.

E34. The method of E, wherein the plurality of pathogens or infectious agents are HIV-1, HIV-2, HCV, HBV, Dengue Virus, WNV, and Chikungunya Virus.

E35. The method of E, wherein the plurality of pathogens or infectious agents are HIV-1, HIV-2, HCV, HBV, and Chikungunya Virus.

E36. The method of E, wherein the plurality of pathogens or infectious agents are HIV-1, HIV-2, HCV, HBV, Chikungunya Virus, and Zika Virus.

E37. The method of E, wherein the plurality of pathogens or infectious agents are HIV-1, HIV-2, HCV, HBV, Chikungunya Virus, Zika Virus, and WNV.

E38. The method of E, wherein the plurality of pathogens or infectious agents are HIV-1, HIV-2, HCV, HBV, WNV, and Dengue Virus.

E39. The method of E, wherein the plurality of pathogens or infectious agents are HIV-1, HIV-2, HCV, HBV, Chikungunya Virus, Zika Virus, and WNV.

E40. The method of E, wherein: the plurality of pathogens or infectious agents are HIV-1, HIV-2, HCV, and HBV; and the nucleic acid analysis comprises multiplex analysis of HIV-1 and HIV-2.

E41. The method of E, wherein: the plurality of pathogens or infectious agents are HIV-1, HIV-2, HCV, and HBV; and the nucleic acid analysis comprises multiplex analysis of HIV-1, HIV-2, and HCV.

E42. The method of E, wherein: the plurality of pathogens or infectious agents are HIV-1, HIV-2, HCV, and HBV; and the nucleic acid analysis comprises multiplex analysis of HIV-1, HIV-2, and HBV.

E43. The method of E, wherein: the plurality of pathogens or infectious agents are HIV-1, HIV-2, HCV, and HBV; and the nucleic acid analysis comprises multiplex analysis of HCV, and HBV.

E44. The method of E, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

E45. The method of E, wherein: the plurality of pathogens or infectious agents are HIV-1, HIV-2, HCV, and HBV; and the nucleic acid analysis comprises multiplex analysis of HIV-1, HIV-2, HCV, and HBV.

E46. The method of E, wherein: the plurality of pathogens or infectious agents comprise Zika Virus, WNV, Chikungunya Virus and Dengue Virus; the nucleic acid analysis comprises multiplex analysis of Zika Virus and WNV.

E47. The method of E, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

E48. The method of E, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

E49. The method of E, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

E50. The method of E, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

E51. The method of E, wherein: the plurality of pathogens or infectious agents comprise Babesia and Malaria; and the nucleic acid analysis comprises multiplex analysis of Babesia and Malaria.

E52. The method of E, wherein: the plurality of pathogens or infectious agents comprise Parvovirus B19 and HAV; and the nucleic acid analysis comprises multiplex analysis of Parvovirus B19 and HAV.

E53. The method of E, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

E54. The method of E, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

E55. The method of E, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

E56. The method of E, wherein the plurality of pathogens or infectious agents are Zika Virus and WNV; and wherein the predetermined levels are: at least 1-50 copies/mL of Zika Virus; and at least 1-50 copies/mL of WNV.

E57. The method of E, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

E58. The method of E, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

E59. The method of E, wherein the plurality of pathogens or infectious agents are Babesia and Malaria; and wherein the predetermined levels are: at least 1-20 copies/mL of Babesia; and at least 1-50 copies/mL of Malaria.

E60. The method of E, wherein the plurality of pathogens or infectious agents are Parvovirus B19 and HAV; and wherein the predetermined levels are: at least 1-40 IU/mL of Parvovirus B19; and at least 1-10 IU/mL of HAV.

E61. The method of E, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

E62. The method of E, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

E63. The method of E, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

E64. The method of E, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

E65. The method of E, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

E66. The method of E, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

E67. The method of E, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

E68. The method of E, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

E69. The method of E, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

E70. The method of E, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

E71. The method of E, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

E72. The method of E, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

E73. The method of E, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

E74. The method of E, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

E75. The method of E, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

E76. The method of E, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

E77. The method of E, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

E78. The method of E, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

E79. The method of E, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

E80. The method of E, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

E81. The method of E, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

E82. The method of E, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

E83. The method of E, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

E84. The method of E, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

E85. The method of E, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

E86. The method of E, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

E87. The method of E, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

E88. The method of E, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

E89. The method of E, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

E90. The method of E, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

E91. The method of E, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

E92. The method of E, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

E93. The method of E, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

E94. The method of E, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

E95. The method of E, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

E96. The method of E, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

E97. The method of E, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

E98. The method of E, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

E99. The method of E, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

E100. The method of E, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

E101. The method of E, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

E102. The method of any one of E-E101, wherein the sample of donor blood is human donor blood.

E103. The method of any one of E-E101, wherein the sample of donor blood is whole blood.

E104. The method of any one of E-E101, wherein the sample of donor blood is lysed whole blood.

E105. The method of any one of E-E101, wherein the sample of donor blood is serum.

E106. The method of any one of E-E101, wherein the sample of donor blood is plasma.

E107. The method of E and E2-E106, wherein the release of the donor material is for transfusion.

E108. The method of E and E2-E106, wherein the release of the donor material is for use in a pharmaceutical.

E109. The method of E and E2-E106, wherein the release of the donor material is for use in a therapeutic treatment.

E110. The method of E and E2-E106, wherein the sample of donor blood is for use as a blood donation.

E111. The method of E and E2-E106, wherein the clinical use is an in vivo clinical use.

E112. The method of E and E2-E106, wherein the clinical use is an in vitro clinical use.

E113. The method of E, E3-E106 and E112, wherein the clinical use is use in disease diagnostics or in quality assurance/laboratory diagnostics.

E114. The method of E-E113, wherein the nucleic acid analysis comprises a nucleic acid amplification reaction.

E115. The method of E114, wherein the nucleic acid amplification reaction is an isothermal reaction.

E116. The method of E115, wherein the isothermal reaction is recombinase polymerase amplification.

E117. The method of E115, wherein the isothermal reaction is a nicking enzyme amplification reaction.

E118. The method of E-E117, wherein the nucleic acid analysis comprises optical detection of the presence of a nucleic acid derived from at least one of the plurality of pathogens or infectious agents.

E119. The method of E-E117, wherein the nucleic acid analysis comprises digital detection of the presence of a nucleic acid derived from at least one of the plurality of pathogens or infectious agents.

E120. The method of E-E119, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

E121. The method of E120, wherein the plurality of times comprises optically detecting the signal at a predetermined interval of about every 20 seconds or about every 30 seconds.

E122. The method of E120 and E121, wherein the optically detecting a fluorescent signal for a plurality of times occurs over a period of about 12 minutes.

F. The present disclosure provides a method of screening samples of donor blood for release of a donor material for clinical use, comprising: performing nucleic acid analysis on the samples of donor blood on an automated system to detect a plurality of pathogens or infectious agents; wherein a determination of a predetermined level of nucleic acids derived from each of the plurality of pathogens or infectious agents based on the nucleic acid analyses, is indicative of release of the donor material for clinical use, and wherein at least about 70 results are obtained per hour per m³ of a volume occupied by the automated system.

F1. The method of F, wherein the determination of a level equal to or greater than the predetermined level indicates that the donor material is not released for clinical use.

F2. The method of F, wherein the determination of a level less than the predetermined level indicates that the donor material is released for clinical use.

F3. The method of any one of F-F3, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

F4. The method of F, wherein the plurality of pathogens or infectious agents are HIV-1, HIV-2, HCV, and HBV.

F5. The method of F, wherein the plurality of pathogens or infectious agents are HIV-1, HIV-2, HCV, HBV, and WNV.

F6. The method of F, wherein the plurality of pathogens or infectious agents are Zika Virus and WNV.

F7. The method of F, wherein the plurality of pathogens or infectious agents are Chikungunya Virus and Dengue Virus.

F8. The method of F, wherein the plurality of pathogens or infectious agents are Zika Virus, WNV, Chikungunya Virus and Dengue Virus.

F9. The method of F, wherein the plurality of pathogens or infectious agents are Babesia and Malaria.

F10. The method of F, wherein the plurality of pathogens or infectious agents are Parvovirus B19 and HAV.

F11. The method of F, wherein the plurality of pathogens or infectious agents are HIV-1, HIV-2, HCV, HBV, WNV, and Zika Virus.

F12. The method of F, wherein the plurality of pathogens or infectious agents are HIV-1, HIV-2, HCV, HBV, WNV, Zika Virus, and Chikungunya Virus.

F13. The method of F, wherein the plurality of pathogens or infectious agents are HIV-1, HIV-2, HCV, HBV, WNV, Zika Virus, Chikungunya Virus, and Dengue Virus.

F14. The method of F, wherein the plurality of pathogens or infectious agents are HIV-1, HIV-2, HCV, HBV, WNV, Zika Virus, Chikungunya Virus, Dengue Virus, and Babesia.

F15. The method of F, wherein the plurality of pathogens or infectious agents are HIV-1, HIV-2, HCV, HBV, WNV, Zika Virus, Chikungunya Virus, Dengue Virus, Babesia, and Malaria.

F16. The method of F, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

F17. The method of F, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

F18. The method of F, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

F19. The method of F, wherein the plurality of pathogens or infectious agents are HIV-1, HIV-2, HCV, HBV, and Parvovirus B19.

F20. The method of F, wherein the plurality of pathogens or infectious agents are HIV-1, HIV-2, HCV, HBV, Parvovirus B19, and HAV.

F21. The method of F, wherein the plurality of pathogens or infectious agents are HIV-1, HIV-2, HCV, HBV, and Babesia.

F22. The method of F, wherein the plurality of pathogens or infectious agents are HIV-1, HIV-2, HCV, HBV, and HAV.

F23. The method of F, wherein the plurality of pathogens or infectious agents are HIV-1, HIV-2, HCV, HBV, and HEV.

F24. The method of F, wherein the plurality of pathogens or infectious agents are HIV-1, HIV-2, HCV, HBV, and Zika Virus.

F25. The method of F, wherein the plurality of pathogens or infectious agents are HIV-1, HIV-2, HCV, HBV, Zika Virus, and Dengue Virus.

F26. The method of F, wherein the plurality of pathogens or infectious agents are HIV-1, HIV-2, HCV, HBV, Zika Virus, Dengue Virus, and Chikungunya Virus.

F27. The method of F, wherein the plurality of pathogens or infectious agents are HIV-1, HIV-2, HCV, HBV, Zika Virus, Dengue Virus, and WNV.

F28. The method of F, wherein the plurality of pathogens or infectious agents are HIV-1, HIV-2, HCV, HBV, Zika Virus, WNV, and Chikungunya Virus.

F29. The method of F, wherein the plurality of pathogens or infectious agents are HIV-1, HIV-2, HCV, HBV, Zika Virus, WNV, Dengue Virus, and Chikungunya Virus.

F30. The method of F, wherein the plurality of pathogens or infectious agents are HIV-1, HIV-2, HCV, HBV, and Malaria.

F31. The method of F, wherein the plurality of pathogens or infectious agents are HIV-1, HIV-2, HCV, HBV, Malaria, and Babesia.

F32. The method of F, wherein the plurality of pathogens or infectious agents are HIV-1, HIV-2, HCV, HBV, and Dengue Virus.

F33. The method of F, wherein the plurality of pathogens or infectious agents are HIV-1, HIV-2, HCV, HBV, Dengue Virus, and Chikungunya Virus.

F34. The method of F, wherein the plurality of pathogens or infectious agents are HIV-1, HIV-2, HCV, HBV, Dengue Virus, WNV, and Chikungunya Virus.

F35. The method of F, wherein the plurality of pathogens or infectious agents are HIV-1, HIV-2, HCV, HBV, and Chikungunya Virus.

F36. The method of F, wherein the plurality of pathogens or infectious agents are HIV-1, HIV-2, HCV, HBV, Chikungunya Virus, and Zika Virus.

F37. The method of F, wherein the plurality of pathogens or infectious agents are HIV-1, HIV-2, HCV, HBV, Chikungunya Virus, Zika Virus, and WNV.

F38. The method of F, wherein the plurality of pathogens or infectious agents are HIV-1, HIV-2, HCV, HBV, WNV, and Dengue Virus.

F39. The method of F, wherein the plurality of pathogens or infectious agents are HIV-1, HIV-2, HCV, HBV, Chikungunya Virus, Zika Virus, and WNV.

F40. The method of F, wherein: the plurality of pathogens or infectious agents are HIV-1, HIV-2, HCV, and HBV; and the nucleic acid analysis comprises multiplex analysis of HIV-1 and HIV-2.

F41. The method of F, wherein: the plurality of pathogens or infectious agents are HIV-1, HIV-2, HCV, and HBV; and the nucleic acid analysis comprises multiplex analysis of HIV-1, HIV-2, and HCV.

F42. The method of F, wherein: the plurality of pathogens or infectious agents are HIV-1, HIV-2, HCV, and HBV; and the nucleic acid analysis comprises multiplex analysis of HIV-1, HIV-2, and HBV.

F43. The method of F, wherein: the plurality of pathogens or infectious agents are HIV-1, HIV-2, HCV, and HBV; and the nucleic acid analysis comprises multiplex analysis of HCV, and HBV.

F44. The method of F, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

F45. The method of F, wherein: the plurality of pathogens or infectious agents are HIV-1, HIV-2, HCV, and HBV; and the nucleic acid analysis comprises multiplex analysis of HIV-1, HIV-2, HCV, and HBV.

F46. The method of F, wherein: the plurality of pathogens or infectious agents comprise Zika Virus, WNV, Chikungunya Virus and Dengue Virus; the nucleic acid analysis comprises multiplex analysis of Zika Virus and WNV.

F47. The method of F, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

F48. The method of F, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

F49. The method of F, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

F50. The method of F, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

F51. The method of F, wherein: the plurality of pathogens or infectious agents comprise Babesia and Malaria; and the nucleic acid analysis comprises multiplex analysis of Babesia and Malaria.

F52. The method of F, wherein: the plurality of pathogens or infectious agents comprise Parvovirus B19 and HAV; and the nucleic acid analysis comprises multiplex analysis of Parvovirus B19 and HAV.

F53. The method of F, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

F54. The method of F, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

F55. The method of F, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

F56. The method of F, wherein the plurality of pathogens or infectious agents are Zika Virus and WNV; and wherein the predetermined levels are: at least 1-50 copies/mL of Zika Virus; and at least 1-50 copies/mL of WNV.

F57. The method of F, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

F58. The method of F, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

F59. The method of F, wherein the plurality of pathogens or infectious agents are Babesia and Malaria; and wherein the predetermined levels are: at least 1-20 copies/mL of Babesia; and at least 1-50 copies/mL of Malaria.

F60. The method of F, wherein the plurality of pathogens or infectious agents are Parvovirus B19 and HAV; and wherein the predetermined levels are: at least 1-40 IU/mL of Parvovirus B19; and at least 1-10 IU/mL of HAV.

F61. The method of F, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

F62. The method of F, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

F63. The method of F, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

F64. The method of F, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

F65. The method of F, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

F66. The method of F, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

F67. The method of F, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

F68. The method of F, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

F69. The method of F, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

F70. The method of F, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

F71. The method of F, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

F72. The method of F, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

F73. The method of F, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

F74. The method of F, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

F75. The method of F, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

F76. The method of F, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

F77. The method of F, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

F78. The method of F, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

F79. The method of F, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

F80. The method of F, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

F81. The method of F, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

F82. The method of F, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

F83. The method of F, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

F84. The method of F, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

F85. The method of F, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

F86. The method of F, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

F87. The method of F, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

F88. The method of F, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

F89. The method of F, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

F90. The method of F, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

F91. The method of F, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

F92. The method of F, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

F93. The method of F, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

F94. The method of F, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

F95. The method of F, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

F96. The method of F, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

F97. The method of F, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

F98. The method of F, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

F99. The method of F, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

F100. The method of F, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

F101. The method of F, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

F102. The method of F, wherein the sample of donor blood is human donor blood.

F103. The method of F, wherein the sample of donor blood is whole blood.

F104. The method of F, wherein the sample of donor blood is lysed whole blood.

F105. The method of F, wherein the sample of donor blood is serum.

F106. The method of F, wherein the sample of donor blood is plasma.

F107. The method of F and F2-F106, wherein the release of the donor material is for transfusion.

F108. The method of F and F2-F106, wherein the release of the donor material is for use in a pharmaceutical.

F109. The method of F and F2-F106, wherein the release of the donor material is for use in a therapeutic treatment.

F110. The method of F and F2-F106, wherein the sample of donor blood is for use as a blood donation.

F111. The method of F and F2-F106, wherein the clinical use is an in vivo clinical use.

F112. The method of F and F2-F106, wherein the clinical use is an in vitro clinical use.

F113. The method of F, F2-F106, wherein the clinical use is use in disease diagnostics or in quality assurance/laboratory diagnostics.

F114. The method of F-F113, wherein the nucleic acid analysis comprises a nucleic acid amplification reaction.

F115. The method of F114, wherein the nucleic acid amplification reaction is an isothermal reaction.

F116. The method of F115, wherein the isothermal reaction is recombinase polymerase amplification.

F117. The method of F115, wherein the isothermal reaction is a nicking enzyme amplification reaction.

F118. The method of F-F117, wherein the nucleic acid analysis comprises optical detection of the presence of a nucleic acid derived from at least one of the plurality of pathogens or infectious agents.

F119. The method of F-F117, wherein the nucleic acid analysis comprises digital detection of the presence of a nucleic acid derived from at least one of the plurality of pathogens or infectious agents.

F120. The method of F-F119, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

F121. The method of F120, wherein the plurality of times comprises optically detecting the signal at a predetermined interval of about every 20 seconds or about every 30 seconds.

F127. The method of F119 and F120, wherein the optically detecting a fluorescent signal for a plurality of times occurs over a period of about 12 minutes.

G. The present disclosure provides a method of screening samples of donor blood for release of a donor material for clinical use, comprising: performing nucleic acid analysis on the samples of donor blood on an automated system to detect a plurality of pathogens or infectious agents; wherein a determination of a predetermined level of nucleic acids derived from each of the plurality of pathogens or infectious agents based on the nucleic acid analysis, is indicative of release of the donor material for clinical use, and wherein at least about 140 results are obtained per hour per m² of a footprint of the automated system.

G1. The method of G, wherein the determination of a level equal to or greater than the predetermined level indicates that the donor material is not released for clinical use.

G2. The method of G, wherein the determination of a level less than the predetermined level indicates that the donor material is released for clinical use.

G3. The method of any one of G-G2, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

G4. The method of G, wherein the plurality of pathogens or infectious agents are HIV-1, HIV-2, HCV, and HBV.

G5. The method of G, wherein the plurality of pathogens or infectious agents are HIV-1, HIV-2, HCV, HBV, and WNV.

G6. The method of G, wherein the plurality of pathogens or infectious agents are Zika Virus and WNV.

G7. The method of G, wherein the plurality of pathogens or infectious agents are Chikungunya Virus and Dengue Virus.

G8. The method of G, wherein the plurality of pathogens or infectious agents are Zika Virus, WNV, Chikungunya Virus and Dengue Virus.

G9. The method of G, wherein the plurality of pathogens or infectious agents are Babesia and Malaria.

G10. The method of G, wherein the plurality of pathogens or infectious agents are Parvovirus B19 and HAV.

G11. The method of G, wherein the plurality of pathogens or infectious agents are HIV-1, HIV-2, HCV, HBV, WNV, and Zika Virus.

G12. The method of G, wherein the plurality of pathogens or infectious agents are HIV-1, HIV-2, HCV, HBV, WNV, Zika Virus, and Chikungunya Virus.

G13. The method of G, wherein the plurality of pathogens or infectious agents are HIV-1, HIV-2, HCV, HBV, WNV, Zika Virus, Chikungunya Virus, and Dengue Virus.

G14. The method of G, wherein the plurality of pathogens or infectious agents are HIV-1, HIV-2, HCV, HBV, WNV, Zika Virus, Chikungunya Virus, Dengue Virus, and Babesia.

G15. The method of G, wherein the plurality of pathogens or infectious agents are HIV-1, HIV-2, HCV, HBV, WNV, Zika Virus, Chikungunya Virus, Dengue Virus, Babesia, and Malaria.

G16. The method of G, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

G17. The method of G, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

G18. The method of G, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

G19. The method of G, wherein the plurality of pathogens or infectious agents are HIV-1, HIV-2, HCV, HBV, and Parvovirus B19.

G20. The method of G, wherein the plurality of pathogens or infectious agents are HIV-1, HIV-2, HCV, HBV, Parvovirus B19, and HAV.

G21. The method of G, wherein the plurality of pathogens or infectious agents are HIV-1, HIV-2, HCV, HBV, and Babesia.

G22. The method of G, wherein the plurality of pathogens or infectious agents are HIV-1, HIV-2, HCV, HBV, and HAV.

G23. The method of G, wherein the plurality of pathogens or infectious agents are HIV-1, HIV-2, HCV, HBV, and HEV.

G24. The method of G, wherein the plurality of pathogens or infectious agents are HIV-1, HIV-2, HCV, HBV, and Zika Virus.

G25. The method of G, wherein the plurality of pathogens or infectious agents are HIV-1, HIV-2, HCV, HBV, Zika Virus, and Dengue Virus.

G26. The method of G, wherein the plurality of pathogens or infectious agents are HIV-1, HIV-2, HCV, HBV, Zika Virus, Dengue Virus, and Chikungunya Virus.

G27. The method of G, wherein the plurality of pathogens or infectious agents are HIV-1, HIV-2, HCV, HBV, Zika Virus, Dengue Virus, and WNV.

G28. The method of G, wherein the plurality of pathogens or infectious agents are HIV-1, HIV-2, HCV, HBV, Zika Virus, WNV, and Chikungunya Virus.

G29. The method of G, wherein the plurality of pathogens or infectious agents are HIV-1, HIV-2, HCV, HBV, Zika Virus, WNV, Dengue Virus, and Chikungunya Virus.

G30. The method of G, wherein the plurality of pathogens or infectious agents are HIV-1, HIV-2, HCV, HBV, and Malaria.

G31. The method of G, wherein the plurality of pathogens or infectious agents are HIV-1, HIV-2, HCV, HBV, Malaria, and Babesia.

G32. The method of G, wherein the plurality of pathogens or infectious agents are HIV-1, HIV-2, HCV, HBV, and Dengue Virus.

G33. The method of G, wherein the plurality of pathogens or infectious agents are HIV-1, HIV-2, HCV, HBV, Dengue Virus, and Chikungunya Virus.

G34. The method of G, wherein the plurality of pathogens or infectious agents are HIV-1, HIV-2, HCV, HBV, Dengue Virus, WNV, and Chikungunya Virus.

G35. The method of G, wherein the plurality of pathogens or infectious agents are HIV-1, HIV-2, HCV, HBV, and Chikungunya Virus.

G36. The method of G, wherein the plurality of pathogens or infectious agents are HIV-1, HIV-2, HCV, HBV, Chikungunya Virus, and Zika Virus.

G37. The method of G, wherein the plurality of pathogens or infectious agents are HIV-1, HIV-2, HCV, HBV, Chikungunya Virus, Zika Virus, and WNV.

G38. The method of G, wherein the plurality of pathogens or infectious agents are HIV-1, HIV-2, HCV, HBV, WNV, and Dengue Virus.

G39. The method of G, wherein the plurality of pathogens or infectious agents are HIV-1, HIV-2, HCV, HBV, Chikungunya Virus, Zika Virus, and WNV.

G40. The method of G, wherein: the plurality of pathogens or infectious agents are HIV-1, HIV-2, HCV, and HBV; and the nucleic acid analysis comprises multiplex analysis of HIV-1 and HIV-2.

G41. The method of G, wherein: the plurality of pathogens or infectious agents are HIV-1, HIV-2, HCV, and HBV; and the nucleic acid analysis comprises multiplex analysis of HIV-1, HIV-2, and HCV.

G42. The method of G, wherein: the plurality of pathogens or infectious agents are HIV-1, HIV-2, HCV, and HBV; and the nucleic acid analysis comprises multiplex analysis of HIV-1, HIV-2, and HBV.

G43. The method of G, wherein: the plurality of pathogens or infectious agents are HIV-1, HIV-2, HCV, and HBV; and the nucleic acid analysis comprises multiplex analysis of HCV, and HBV.

G44. The method of G, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

G45. The method of G, wherein: the plurality of pathogens or infectious agents are HIV-1, HIV-2, HCV, and HBV; and the nucleic acid analysis comprises multiplex analysis of HIV-1, HIV-2, HCV, and HBV.

G46. The method of G, wherein: the plurality of pathogens or infectious agents comprise Zika Virus, WNV, Chikungunya Virus and Dengue Virus; the nucleic acid analysis comprises multiplex analysis of Zika Virus and WNV.

G47. The method of G, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

G48. The method of G, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

G49. The method of G, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

G50. The method of G, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

G51. The method of G, wherein: the plurality of pathogens or infectious agents comprise Babesia and Malaria; and the nucleic acid analysis comprises multiplex analysis of Babesia and Malaria.

G52. The method of G, wherein: the plurality of pathogens or infectious agents comprise Parvovirus B19 and HAV; and the nucleic acid analysis comprises multiplex analysis of Parvovirus B19 and HAV.

G53. The method of G, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

G54. The method of G, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

G55. The method of G, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

G56. The method of G, wherein the plurality of pathogens or infectious agents are Zika Virus and WNV; and wherein the predetermined levels are: at least 1-50 copies/mL of Zika Virus; and at least 1-50 copies/mL of WNV.

G57. The method of G, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

G58. The method of G, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

G59. The method of G, wherein the plurality of pathogens or infectious agents are Babesia and Malaria; and wherein the predetermined levels are: at least 1-20 copies/mL of Babesia; and at least 1-50 copies/mL of Malaria.

G60. The method of G, wherein the plurality of pathogens or infectious agents are Parvovirus B19 and HAV; and wherein the predetermined levels are: at least 1-40 IU/mL of Parvovirus B19; and at least 1-10 IU/mL of HAV.

G61. The method of G, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

G62. The method of G, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

G63. The method of G, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

G64. The method of G, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

G65. The method of G, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

G66. The method of G, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

G67. The method of G, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

G68. The method of G, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

G69. The method of G, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

G70. The method of G, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

G71. The method of G, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

G72. The method of G, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

G73. The method of G, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

G74. The method of G, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

G75. The method of G, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

G76. The method of G, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

G77. The method of G, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

G78. The method of G, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

G79. The method of G, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

G80. The method of G, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

G81. The method of G, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

G82. The method of G, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

G83. The method of G, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

G84. The method of G, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

G85. The method of G, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

G86. The method of G, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

G87. The method of G, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

G88. The method of G, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

G89. The method of G, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

G90. The method of G, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

G91. The method of G, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

G92. The method of G, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

G93. The method of G, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

G94. The method of G, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

G95. The method of G, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

G96. The method of G, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

G97. The method of G, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

G98. The method of G, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

G99. The method of G, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

G100. The method of G, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

G101. The method of G, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

G102. The method of any one of G-G101, wherein the sample of donor blood is human donor blood.

G103. The method of any one of G-G101, wherein the sample of donor blood is whole blood.

G104. The method of any one of G-G101, wherein the sample of donor blood is lysed whole blood.

G105. The method of any one of G-G101, wherein the sample of donor blood is serum.

G106. The method of any one of G-G101, wherein the sample of donor blood is plasma.

G107. The method of G and G2-G106, wherein the release of the donor material is for transfusion.

G108. The method of G and G2-G106, wherein the release of the donor material is for use in a pharmaceutical.

G109. The method of G and G2-G106, wherein the release of the donor material is for use in a therapeutic treatment.

G110. The method of G and G2-G106, wherein the sample of donor blood is for use as a blood donation.

G111. The method of G and G3-G106, wherein the clinical use is an in vivo clinical use.

G112. The method of G and G3-G106, wherein the clinical use is an in vitro clinical use.

G113. The method of G and G3-G106, wherein the clinical use is use in disease diagnostics or in quality assurance/laboratory diagnostics.

G114. The method of G-G114, wherein the nucleic acid analysis comprises a nucleic acid amplification reaction.

G115. The method of G115, wherein the nucleic acid amplification reaction is an isothermal reaction.

G116. The method of G116, wherein the isothermal reaction is recombinase polymerase amplification.

G117. The method of G116, wherein the isothermal reaction is a nicking enzyme amplification reaction.

G118. The method of G-G117, wherein the nucleic acid analysis comprises optical detection of the presence of a nucleic acid derived from at least one of the plurality of pathogens or infectious agents.

G119. The method of G-G117, wherein the nucleic acid analysis comprises digital detection of the presence of a nucleic acid derived from at least one of the plurality of pathogens or infectious agents.

G120. The method of G-G119, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

G121. The method of G120, wherein the plurality of times comprises optically detecting the signal at a predetermined interval of about every 20 seconds or about every 30 seconds.

G122. The method of G119 and G120, wherein the optically detecting a fluorescent signal for a plurality of times occurs over a period of about 12 minutes.

H. The present disclosure provides a method of screening a sample of donor blood for release of a donor material for clinical use, comprising: performing a nucleic acid analysis on the sample of donor blood to detect a plurality of pathogens or infectious agents; wherein a determination of a predetermined level of nucleic acids derived from each of the plurality of pathogens or infectious agents based on the nucleic acid analysis, is indicative of release of the donor material for clinical use, and wherein, upon a determination of the presence of a nucleic acid derived from at least one of the plurality of pathogens or infectious agents at or in excess of the predetermined level in a pooled sample, the methods further comprising screening samples of individual donor blood or sub-pools thereof included in the pooled sample, comprising: performing a nucleic acid analysis on the samples of donor blood to detect a plurality of pathogens or infectious agents; wherein a determination of a predetermined level of nucleic acids derived from each of the plurality of pathogens or infectious agents based on the nucleic acid analysis, is indicative of release of the donor material for clinical use.

H1. The method of H, wherein the determination of a level equal to or greater than the predetermined level indicates that the donor material is not released for clinical use.

H2. The method of H, wherein the determination of a level less than the predetermined level indicates that the donor material is released for clinical use.

H3. The method of any one of H-H2, wherein the sample of donor blood is human donor blood.

H4. The method of any one of H-H2, wherein the sample of donor blood is whole blood.

H5. The method of any one of H-H2, wherein the sample of donor blood is lysed whole blood.

H6. The method of any one of H-H2, wherein the sample of donor blood is serum.

H7. The method of any one of H-H2, wherein the sample of donor blood is plasma.

H8. The method of H and H2-H7, wherein the release of the donor material is for transfusion.

H9. The method of H and H2-H7, wherein the release of the donor material is for use in a pharmaceutical.

H10. The method of H and H2-H7, wherein the release of the donor material is for use in a therapeutic treatment.

H11. The method of H and H2-H7, wherein the sample of donor blood is for use as a blood donation.

H12. The method of H and H2-H7, wherein the clinical use is an in vivo clinical use.

H13. The method of H and H2-H7, wherein the clinical use is an in vitro clinical use.

H19. The method of any one of H and H2-H7, wherein the clinical use is use in disease diagnostics or in quality assurance/laboratory diagnostics.

H20. The method of H-H19, wherein the nucleic acid analysis comprises a nucleic acid amplification reaction.

H21. The method of H20, wherein the nucleic acid amplification reaction is an isothermal reaction.

H22. The method of H21, wherein the isothermal reaction is recombinase polymerase amplification.

H23. The method of H21, wherein the isothermal reaction is a nicking enzyme amplification reaction.

H24. The method of H-H23, wherein the nucleic acid analysis comprises optical detection of the presence of a nucleic acid derived from at least one of the plurality of pathogens or infectious agents.

H25. The method of H-H24, wherein the nucleic acid analysis comprises digital detection of the presence of a nucleic acid derived from at least one of the plurality of pathogens or infectious agents.

H26. The method of H-H25, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

H27. The method of H26, wherein the plurality of times comprises optically detecting the signal at a predetermined interval of about every 20 seconds or about every 30 seconds.

H28. The method of H26 and H27, wherein the optically detecting a fluorescent signal for a plurality of times occurs over a period of about 12 minutes.

I. The present disclosure provides a method of screening a sample associated with donor blood for release of a donor material for clinical use, comprising: performing a nucleic acid analysis on the sample, comprising: a sample preparation process comprising providing nucleic acid isolated from the sample in a volume of eluate; dispensing the volume of eluate into at least two amplification vessels; performing an amplification process on the eluate in the at least two amplification vessels; and performing a detection process to detect whether nucleic acid derived from one or more pathogens or infectious agents is present in the eluate.

I1. The method of I, wherein the detection process includes detecting whether nucleic acid derived from a plurality of pathogens or infections agents is present or absent in the eluate.

I2. The method of I-I1, wherein the dispensing comprises dispensing the volume of eluate into at least three, four, or more amplification vessels; subjecting the eluate in the amplification vessels to a nucleic acid amplification reaction; and detecting whether nucleic acid present in a pathogen or an infectious agent is present in the eluate.

I3. The method of I-I2, wherein an equal volume of eluate is dispensed into the amplification vessels.

I14. The method of I-I2, wherein different volumes of the eluate are dispensed into the amplification vessels.

I15. The method of I-I4, wherein the amplification reaction in each amplification vessels is for amplification of nucleic acid present in a different pathogen or infectious agent.

I16. The method of I-I5, wherein the amplification reaction in each amplification vessel is for amplification of nucleic acid present in a plurality of different pathogens or infectious agents.

I17. The method of I-I6, wherein the amplification reaction comprises an isothermal reaction.

I18. The method of I7, wherein the isothermal reaction is recombinase polymerase amplification.

I19. The method of I7, wherein the isothermal reaction is a nicking enzyme amplification reaction.

I110. The method of any one of I and I1-9, wherein the determination of a level equal to or greater than the predetermined level indicates that the donor material is not released for clinical use.

I11. The method of any one of I and I1-I9, wherein the determination of a level less than the predetermined level indicates that the donor material, donor blood, biological sample, or plasma is released for clinical use.

I12. The method of any one of I-I11, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

I13. The method of any one of I-I11, wherein the plurality of pathogens or infectious agents are HIV-1, HIV-2, HCV, and HBV.

I14. The method of any one of I-I11, wherein the plurality of pathogens or infectious agents are HIV-1, HIV-2, HCV, HBV, and WNV.

I15. The method of any one of I-I11, wherein the plurality of pathogens or infectious agents are Zika Virus and WNV.

I16. The method of any one of I-I11, wherein the plurality of pathogens or infectious agents are Chikungunya Virus and Dengue Virus.

I17. The method of any one of I-I11, wherein the plurality of pathogens or infectious agents are Zika Virus, WNV, Chikungunya Virus and Dengue Virus.

I18. The method of any one of I-I11, wherein the plurality of pathogens or infectious agents are Babesia and Malaria.

I19. The method of any one of I-I11, wherein the plurality of pathogens or infectious agents are Parvovirus B19 and HAV.

I120. The method of any one of I-I11, wherein the plurality of pathogens or infectious agents are HIV-1, HIV-2, HCV, HBV, WNV, and Zika Virus.

I121. The method of any one of I-I11, wherein the plurality of pathogens or infectious agents are HIV-1, HIV-2, HCV, HBV, WNV, Zika Virus, and Chikungunya Virus.

I122. The method of any one of I-I11, wherein the plurality of pathogens or infectious agents are HIV-1, HIV-2, HCV, HBV, WNV, Zika Virus, Chikungunya Virus, and Dengue Virus.

I123. The method of any one of I-I11, wherein the plurality of pathogens or infectious agents are HIV-1, HIV-2, HCV, HBV, WNV, Zika Virus, Chikungunya Virus, Dengue Virus, and Babesia.

I124. The method of any one of I-I11, wherein the plurality of pathogens or infectious agents are HIV-1, HIV-2, HCV, HBV, WNV, Zika Virus, Chikungunya Virus, Dengue Virus, Babesia, and Malaria.

I125. The method of any one of I-I11, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

I26. The method of any one of I-I11, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

I127. The method of any one of I-I11, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

I128. The method of any one of I-I11, wherein the plurality of pathogens or infectious agents are HIV-1, HIV-2, HCV, HBV, and Parvovirus B19.

I29. The method of any one of I-I11, wherein the plurality of pathogens or infectious agents are HIV-1, HIV-2, HCV, HBV, Parvovirus B19, and HAV.

I130. The method of any one of I-I11, wherein the plurality of pathogens or infectious agents are HIV-1, HIV-2, HCV, HBV, and Babesia.

I131. The method of any one of I-I11, wherein the plurality of pathogens or infectious agents are HIV-1, HIV-2, HCV, HBV, and HAV.

I132. The method of any one of I-I11, wherein the plurality of pathogens or infectious agents are HIV-1, HIV-2, HCV, HBV, and HEV.

I133. The method of any one of I-I11, wherein the plurality of pathogens or infectious agents are HIV-1, HIV-2, HCV, HBV, and Zika Virus.

I134. The method of any one of I-I11, wherein the plurality of pathogens or infectious agents are HIV-1, HIV-2, HCV, HBV, Zika Virus, and Dengue Virus.

I135. The method of any one of I-I11, wherein the plurality of pathogens or infectious agents are HIV-1, HIV-2, HCV, HBV, Zika Virus, Dengue Virus, and Chikungunya Virus.

I136. The method of any one of I-I11, wherein the plurality of pathogens or infectious agents are HIV-1, HIV-2, HCV, HBV, Zika Virus, Dengue Virus, and WNV.

I137. The method of any one of I-I11, wherein the plurality of pathogens or infectious agents are HIV-1, HIV-2, HCV, HBV, Zika Virus, WNV, and Chikungunya Virus.

I138. The method of any one of I-I11, wherein the plurality of pathogens or infectious agents are HIV-1, HIV-2, HCV, HBV, Zika Virus, WNV, Dengue Virus, and Chikungunya Virus.

I139. The method of any one of I-I11, wherein the plurality of pathogens or infectious agents are HIV-1, HIV-2, HCV, HBV, and Malaria.

I140. The method of any one of I-I11, wherein the plurality of pathogens or infectious agents are HIV-1, HIV-2, HCV, HBV, Malaria, and Babesia.

I141. The method of any one of I-I11, wherein the plurality of pathogens or infectious agents are HIV-1, HIV-2, HCV, HBV, and Dengue Virus.

I142. The method of any one of I-I11, wherein the plurality of pathogens or infectious agents are HIV-1, HIV-2, HCV, HBV, Dengue Virus, and Chikungunya Virus.

I143. The method of any one of I-I11, wherein the plurality of pathogens or infectious agents are HIV-1, HIV-2, HCV, HBV, Dengue Virus, WNV, and Chikungunya Virus.

I144. The method of any one of I-I11, wherein the plurality of pathogens or infectious agents are HIV-1, HIV-2, HCV, HBV, and Chikungunya Virus.

I145. The method of any one of I-I11, wherein the plurality of pathogens or infectious agents are HIV-1, HIV-2, HCV, HBV, Chikungunya Virus, and Zika Virus.

I146. The method of any one of I-I11, wherein the plurality of pathogens or infectious agents are HIV-1, HIV-2, HCV, HBV, Chikungunya Virus, Zika Virus, and WNV.

I147. The method of any one of I-I11, wherein the plurality of pathogens or infectious agents are HIV-1, HIV-2, HCV, HBV, WNV, and Dengue Virus.

I148. The method of any one of I-I11, wherein the plurality of pathogens or infectious agents are HIV-1, HIV-2, HCV, HBV, Chikungunya Virus, Zika Virus, and WNV.

I149. The method of any one of I-I11, wherein: the plurality of pathogens or infectious agents are HIV-1, HIV-2, HCV, and HBV; and the nucleic acid analysis comprises multiplex analysis of HIV-1 and HIV-2.

I50. The method of any one of I-I11, wherein: the plurality of pathogens or infectious agents are HIV-1, HIV-2, HCV, and HBV; and the nucleic acid analysis comprises multiplex analysis of HIV-1, HIV-2, and HCV.

I151. The method of any one of I-I11, wherein: the plurality of pathogens or infectious agents are HIV-1, HIV-2, HCV, and HBV; and the nucleic acid analysis comprises multiplex analysis of HIV-1, HIV-2, and HBV.

I152. The method of any one of I-I11, wherein: the plurality of pathogens or infectious agents are HIV-1, HIV-2, HCV, and HBV; and the nucleic acid analysis comprises multiplex analysis of HCV, and HBV.

I153. The method of any one of I-I11, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

I154. The method of any one of I-I11, wherein: the plurality of pathogens or infectious agents are HIV-1, HIV-2, HCV, and HBV; and the nucleic acid analysis comprises multiplex analysis of HIV-1, HIV-2, HCV, and HBV.

I155. The method of any one of I-I11, wherein: the plurality of pathogens or infectious agents comprise Zika Virus, WNV, Chikungunya Virus and Dengue Virus; the nucleic acid analysis comprises multiplex analysis of Zika Virus and WNV.

I156. The method of any one of I-I11, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

I157. The method of any one of I-I11, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

I158. The method of any one of I-I11, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

I59. The method of any one of I-I11, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

I160. The method of any one of I-I11, wherein: the plurality of pathogens or infectious agents comprise Babesia and Malaria; and the nucleic acid analysis comprises multiplex analysis of Babesia and Malaria.

I161. The method of any one of I-I11, wherein: the plurality of pathogens or infectious agents comprise Parvovirus B19 and HAV; and the nucleic acid analysis comprises multiplex analysis of Parvovirus B19 and HAV.

I162. The method of any one of I-I11, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

I163. The method of any one of I-I11, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

I164. The method of any one of I-I11, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

I165. The method of any one of I-I11, wherein the plurality of pathogens or infectious agents are Zika Virus and WNV; and wherein the predetermined levels are: at least 1-50 copies/mL of Zika Virus; and at least 1-50 copies/mL of WNV.

I166. The method of any one of I-I11, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

I167. The method of any one of I-I11, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

I168. The method of any one of I-I11, wherein the plurality of pathogens or infectious agents are Babesia and Malaria; and wherein the predetermined levels are: at least 1-20 copies/mL of Babesia; and at least 1-50 copies/mL of Malaria.

I169. The method of any one of I-I11, wherein the plurality of pathogens or infectious agents are Parvovirus B19 and HAV; and wherein the predetermined levels are: at least 1-40 IU/mL of Parvovirus B19; and at least 1-10 IU/mL of HAV.

I170. The method of any one of I-I11, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

I171. The method of any one of I-I11, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

I172. The method of any one of I-I11, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

I173. The method of any one of I-I11, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

I174. The method of any one of I-I11, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

I175. The method of any one of I-I11, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

I76. The method of any one of I-I11, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

I77. The method of any one of I-I11, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

I178. The method of any one of I-I11, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

I79. The method of any one of I-I11 wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

I180. The method of any one of I-I11, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

I181. The method of any one of I-I11, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

I182. The method of any one of I-I11, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

I183. The method of any one of I-I11, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

I184. The method of any one of I-I11, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

I185. The method of any one of I-I11, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

I186. The method of any one of I-I11, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

I187. The method of any one of I-I11, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

I188. The method of any one of I-I11, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

I189. The method of any one of I-I11, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

I190. The method of any one of I-I11, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

I191. The method of any one of I-I11, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

I192. The method of any one of I-I11, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

I193. The method of any one of I-I11, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

I194. The method of any one of I-I11, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

I195. The method of any one of I-I11, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

I196. The method of any one of I-I11, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

I197. The method of any one of I-I11, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

I198. The method of any one of I-I11, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

I199. The method of any one of I-I11, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

I100. The method of any one of I-I11, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

I101. The method of any one of I-I11, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

I102. The method of any one of I-I11, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

I103. The method of any one of I-I11, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

I104. The method of any one of I-I11, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

I105. The method of any one of I-I11, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

I106. The method of any one of I-I11, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

I107. The method of any one of I-I11, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

I108. The method of any one of I-I11, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

I109. The method of any one of I-I11, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

I110. The method of any one of I-I11, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

I111. The method of any one of I-I110, wherein the sample or biological sample is a sample of donor blood.

I112. The method of any one of I-I110, wherein the sample of donor blood is human donor blood.

I113. The method of any one of I-I110, wherein the sample of donor blood is whole blood.

I114. The method of any one of I-I110, wherein the sample of donor blood is lysed whole blood.

I115. The method of any one of I-I110, wherein the sample of donor blood is serum.

I116. The method of any one of I-I110, wherein the sample of donor blood is plasma.

I117. The method of any one of I-I117, wherein the release of the donor material is for transfusion.

I118. The method of any one of I-I117, wherein the release of the donor material is for use in a pharmaceutical.

I119. The method of any one of I-I117, wherein the release of the donor material is for use in a therapeutic treatment.

I120. The method of any one of I-I17, wherein the clinical use is an in vivo clinical use.

I121. The method of any one of I-I17, wherein the clinical use is an in vitro clinical use.

I127. The method of any one of I-I117, wherein the clinical use is use in disease diagnostics or in quality assurance/laboratory diagnostics.

I128. The method of any one of I-I127, wherein the nucleic acid analysis comprises a nucleic acid amplification reaction.

I129. The method of I128, wherein the nucleic acid amplification reaction is an isothermal reaction.

I130. The method of I129, wherein the isothermal reaction is recombinase polymerase amplification.

I131. The method of I129, wherein the isothermal reaction is a nicking enzyme amplification reaction.

I132. The method of any one of I-I31, wherein the nucleic acid analysis comprises optical detection of the presence of a nucleic acid derived from at least one of the plurality of pathogens or infectious agents.

I133. The method of any one of I-I32, wherein the nucleic acid analysis comprises digital detection of the presence of a nucleic acid derived from at least one of the plurality of pathogens or infectious agents.

I134. The method of any one of I-I33, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

I135. The method of I134, wherein the plurality of times comprises optically detecting the signal at a predetermined interval of about every 20 seconds or about every 30 seconds.

I136. The method of I134 and I135, wherein the optically detecting a fluorescent signal for a plurality of times occurs over a period of about 12 minutes.

J. The present disclosure provides a method for screening a plurality of samples to determine whether donor blood associated with the samples is acceptable for transfusion comprising: performing a nucleic acid analysis on the samples including a sample preparation process and an amplification process; determining a result from the nucleic acid analysis; and making a determination whether donor blood is acceptable for transfusion based at least in part on the nucleic acid analysis result wherein release of the donor blood for transfusion occurs in about 15 minutes to about 60 minutes from initial aspiration of the sample for performance of the nucleic acid analysis.

J1. The method of J, wherein the nucleic acid analysis comprises a nucleic acid amplification and detection process of about 1 minute to about 20 minutes in duration.

J1-1. The method of J, wherein release of the donor blood for transfusion occurs in about 20 minutes to about 60 minutes from initial aspiration of the sample for performance of the nucleic acid analysis.

J2. The method of J, J1-1 or J1, wherein the result is completed in about 15 to about 45 minutes from initial aspiration of a sample for performance of the nucleic acid analysis.

J3. The method of any one of J-J2, wherein each result has a time to result of about 20 to about 45 minutes.

J4. The method of any one of J-J3, wherein the nucleic acid analysis is performed on an automated system and wherein at least about 70 results are obtained per hour per m³ of a volume occupied by the automated system.

J5. The method of any one of J-J4, wherein the nucleic acid analysis is performed on an automated system and wherein at least about 140 results are obtained per hour per m² of a footprint of the automated system.

J6. The method of any one of J-J5, wherein each result includes a determination of a predetermined level of nucleic acid from at least one of a plurality of pathogens or infectious agents.

J7. The method of J6, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

J8. The method of J6, wherein the plurality of pathogens or infectious agents are HIV-1, HIV-2, HCV, and HBV.

J9. The method of J6, wherein the plurality of pathogens or infectious agents are HIV-1, HIV-2, HCV, HBV, and WNV.

J10. The method of J6, wherein the plurality of pathogens or infectious agents are Zika Virus and WNV.

J11. The method of J6, wherein the plurality of pathogens or infectious agents are Chikungunya Virus and Dengue Virus.

J12. The method of J6, wherein the plurality of pathogens or infectious agents are Zika Virus, WNV, Chikungunya Virus and Dengue Virus.

J13. The method of J6, wherein the plurality of pathogens or infectious agents are Babesia and Malaria.

J14. The method of J6, wherein the plurality of pathogens or infectious agents are Parvovirus B19 and HAV.

J15. The method of J6, wherein the plurality of pathogens or infectious agents are HIV-1, HIV-2, HCV, HBV, WNV, and Zika Virus.

J16. The method of J6, wherein the plurality of pathogens or infectious agents are HIV-1, HIV-2, HCV, HBV, WNV, Zika Virus, and Chikungunya Virus.

J17. The method of J6, wherein the plurality of pathogens or infectious agents are HIV-1, HIV-2, HCV, HBV, WNV, Zika Virus, Chikungunya Virus, and Dengue Virus.

J18. The method of J6, wherein the plurality of pathogens or infectious agents are HIV-1, HIV-2, HCV, HBV, WNV, Zika Virus, Chikungunya Virus, Dengue Virus, and Babesia.

J19. The method of J6, wherein the plurality of pathogens or infectious agents are HIV-1, HIV-2, HCV, HBV, WNV, Zika Virus, Chikungunya Virus, Dengue Virus, Babesia, and Malaria.

J20. The method of J6, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

J21. The method of J6, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

J22. The method of J6, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

J23. The method of J6, wherein the plurality of pathogens or infectious agents are HIV-1, HIV-2, HCV, HBV, and Parvovirus B19.

J24. The method of J6, wherein the plurality of pathogens or infectious agents are HIV-1, HIV-2, HCV, HBV, Parvovirus B19, and HAV.

J25. The method of J6, wherein the plurality of pathogens or infectious agents are HIV-1, HIV-2, HCV, HBV, and Babesia.

J26. The method of J6, wherein the plurality of pathogens or infectious agents are HIV-1, HIV-2, HCV, HBV, and HAV.

J27. The method of J6, wherein the plurality of pathogens or infectious agents are HIV-1, HIV-2, HCV, HBV, and HEV.

J28. The method of J6, wherein the plurality of pathogens or infectious agents are HIV-1, HIV-2, HCV, HBV, and Zika Virus.

J29. The method of J6, wherein the plurality of pathogens or infectious agents are HIV-1, HIV-2, HCV, HBV, Zika Virus, and Dengue Virus.

J30. The method of J6, wherein the plurality of pathogens or infectious agents are HIV-1, HIV-2, HCV, HBV, Zika Virus, Dengue Virus, and Chikungunya Virus.

J31. The method of J6, wherein the plurality of pathogens or infectious agents are HIV-1, HIV-2, HCV, HBV, Zika Virus, Dengue Virus, and WNV.

J32. The method of J6, wherein the plurality of pathogens or infectious agents are HIV-1, HIV-2, HCV, HBV, Zika Virus, WNV, and Chikungunya Virus.

J33. The method of J6, wherein the plurality of pathogens or infectious agents are HIV-1, HIV-2, HCV, HBV, Zika Virus, WNV, Dengue Virus, and Chikungunya Virus.

J34. The method of J6, wherein the plurality of pathogens or infectious agents are HIV-1, HIV-2, HCV, HBV, and Malaria.

J35. The method of J6, wherein the plurality of pathogens or infectious agents are HIV-1, HIV-2, HCV, HBV, Malaria, and Babesia.

J36. The method of J6, wherein the plurality of pathogens or infectious agents are HIV-1, HIV-2, HCV, HBV, and Dengue Virus.

J37. The method of J6, wherein the plurality of pathogens or infectious agents are HIV-1, HIV-2, HCV, HBV, Dengue Virus, and Chikungunya Virus.

J38. The method of J6, wherein the plurality of pathogens or infectious agents are HIV-1, HIV-2, HCV, HBV, Dengue Virus, WNV, and Chikungunya Virus.

J39. The method of J6, wherein the plurality of pathogens or infectious agents are HIV-1, HIV-2, HCV, HBV, and Chikungunya Virus.

J40. The method of J6, wherein the plurality of pathogens or infectious agents are HIV-1, HIV-2, HCV, HBV, Chikungunya Virus, and Zika Virus.

J41. The method of J6, wherein the plurality of pathogens or infectious agents are HIV-1, HIV-2, HCV, HBV, Chikungunya Virus, Zika Virus, and WNV.

J42. The method of J6, wherein the plurality of pathogens or infectious agents are HIV-1, HIV-2, HCV, HBV, WNV, and Dengue Virus.

J43. The method of J6, wherein the plurality of pathogens or infectious agents are HIV-1, HIV-2, HCV, HBV, Chikungunya Virus, Zika Virus, and WNV.

J44. The method of J6, wherein: the plurality of pathogens or infectious agents are HIV-1, HIV-2, HCV, and HBV; and the nucleic acid analysis comprises multiplex analysis of HIV-1 and HIV-2.

J45. The method of J6, wherein: the plurality of pathogens or infectious agents are HIV-1, HIV-2, HCV, and HBV; and the nucleic acid analysis comprises multiplex analysis of HIV-1, HIV-2, and HCV.

J46. The method of J6, wherein: the plurality of pathogens or infectious agents are HIV-1, HIV-2, HCV, and HBV; and the nucleic acid analysis comprises multiplex analysis of HIV-1, HIV-2, and HBV.

J47. The method of J6, wherein: the plurality of pathogens or infectious agents are HIV-1, HIV-2, HCV, and HBV; and the nucleic acid analysis comprises multiplex analysis of HCV, and HBV.

J48. The method of J6, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

J49. The method of J6, wherein: the plurality of pathogens or infectious agents are HIV-1, HIV-2, HCV, and HBV; and the nucleic acid analysis comprises multiplex analysis of HIV-1, HIV-2, HCV, and HBV.

J50. The method of J6, wherein: the plurality of pathogens or infectious agents comprise Zika Virus, WNV, Chikungunya Virus and Dengue Virus; the nucleic acid analysis comprises multiplex analysis of Zika Virus and WNV.

J51. The method of J6, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

J52. The method of J6, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

J53. The method of J6, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

J54. The method of J6, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

J55. The method of J6, wherein: the plurality of pathogens or infectious agents comprise Babesia and Malaria; and the nucleic acid analysis comprises multiplex analysis of Babesia and Malaria.

J56. The method of J6, wherein: the plurality of pathogens or infectious agents comprise Parvovirus B19 and HAV; and the nucleic acid analysis comprises multiplex analysis of Parvovirus B19 and HAV.

J57. The method of J6, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

J58. The method of J6, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

J59. The method of J6, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

J60. The method of J6, wherein the plurality of pathogens or infectious agents are Zika Virus and WNV; and wherein the predetermined levels are: at least 1-50 copies/mL of Zika Virus; and at least 1-50 copies/mL of WNV.

J61. The method of J6, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

J62. The method of J6, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

J63. The method of J6, wherein the plurality of pathogens or infectious agents are Babesia and Malaria; and wherein the predetermined levels are: at least 1-20 copies/mL of Babesia; and at least 1-50 copies/mL of Malaria.

J64. The method of J6, wherein the plurality of pathogens or infectious agents are Parvovirus B19 and HAV; and wherein the predetermined levels are: at least 1-40 IU/mL of Parvovirus B19; and at least 1-10 IU/mL of HAV.

J65. The method of J6, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

J66. The method of J6, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

J67. The method of J6, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

J68. The method of J6, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

J69. The method of J6, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

J70. The method of J6, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

J71. The method of J6, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

J72. The method of J6, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

J73. The method of J6, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

J74. The method of J6, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

J75. The method of J6, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

J76. The method of J6, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

J77. The method of J6, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

J78. The method of J6, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

J79. The method of J6, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

J80. The method of J6, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

J81. The method of J6, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

J82. The method of J6, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

J83. The method of J6, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

J84. The method of J6, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

J85. The method of J6, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

J86. The method of J6, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

J87. The method of J6, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

J88. The method of J6, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

J89. The method of J6, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

J90. The method of J6, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

J91. The method of J6, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

J92. The method of J6, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

J93. The method of J6, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

J94. The method of J6, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

J95. The method of J6, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

J96. The method of J6, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

J97. The method of J6, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

J98. The method of J6, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

J99. The method of J6, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

J100. The method of J6, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

J101. The method of J6, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

J102. The method of J6, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

J103. The method of J6, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

J104. The method of J6, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

J105. The method of J6, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

J106. The method of J-J5, wherein the sample or biological sample is a sample of donor blood.

J107. The method of any one of J-J106, wherein the sample of donor blood is human donor blood.

J108. The method of any one of J-J106, wherein the sample of donor blood is whole blood.

J109. The method of any one of J-J106, wherein the sample of donor blood is lysed whole blood.

J110. The method of any one of J-J106, wherein the sample of donor blood is serum.

J111. The method of any one of J-J106, wherein the sample of donor blood is plasma.

J112. The method of J-J111, wherein the nucleic acid analysis comprises a nucleic acid amplification reaction.

J113. The method of J112, wherein the nucleic acid amplification reaction is an isothermal reaction.

J114. The method of J113, wherein the isothermal reaction is recombinase polymerase amplification.

J115. The method of J113, wherein the isothermal reaction is a nicking enzyme amplification reaction.

J116. The method of J-J115, wherein the nucleic acid analysis comprises optical detection of the presence of a nucleic acid derived from at least one of the plurality of pathogens or infectious agents.

J117. The method of J-J116, wherein the nucleic acid analysis comprises digital detection of the presence of a nucleic acid derived from at least one of the plurality of pathogens or infectious agents.

J118. The method of J-J117, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

J119. The method of J118, wherein the plurality of times comprises optically detecting the signal at a predetermined interval of about every 20 seconds or about every 30 seconds.

J120. The method of J118 and J119, wherein the optically detecting a fluorescent signal for a plurality of times occurs over a period of about 12 minutes.

K. The present disclosure provides a method for screening a plurality of samples to determine whether donor blood associated with the samples is acceptable for transfusion comprising: performing a nucleic acid analysis on the samples including a sample preparation process and an amplification process; determining a result from the nucleic acid analysis; and making a determination whether donor blood is acceptable for transfusion based at least in part on the nucleic acid analysis result wherein the determinations based on the nucleic acid analyses are performed within about 20 minutes to about 3.5 hours from initial aspiration of the first sample for performance of the nucleic acid analysis.

K1. The method of K, wherein each result includes a determination of a predetermined level of nucleic acid from at least one of a plurality of pathogens or infectious agents.

K2. The method of K or K1, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

K3. The method of K1, wherein the plurality of pathogens or infectious agents are HIV-1, HIV-2, HCV, and HBV.

K4. The method of K1, wherein the plurality of pathogens or infectious agents are HIV-1, HIV-2, HCV, HBV, and WNV.

K5. The method of K1, wherein the plurality of pathogens or infectious agents are Zika Virus and WNV.

K6. The method of K1, wherein the plurality of pathogens or infectious agents are Chikungunya Virus and Dengue Virus.

K7. The method of K1, wherein the plurality of pathogens or infectious agents are Zika Virus, WNV, Chikungunya Virus and Dengue Virus.

K8. The method of K1, wherein the plurality of pathogens or infectious agents are Babesia and Malaria.

K9. The method of K1, wherein the plurality of pathogens or infectious agents are Parvovirus B19 and HAV.

K10. The method of K1, wherein the plurality of pathogens or infectious agents are HIV-1, HIV-2, HCV, HBV, WNV, and Zika Virus.

K11. The method of K1, wherein the plurality of pathogens or infectious agents are HIV-1, HIV-2, HCV, HBV, WNV, Zika Virus, and Chikungunya Virus.

K12. The method of K1, wherein the plurality of pathogens or infectious agents are HIV-1, HIV-2, HCV, HBV, WNV, Zika Virus, Chikungunya Virus, and Dengue Virus.

K13. The method of K1, wherein the plurality of pathogens or infectious agents are HIV-1, HIV-2, HCV, HBV, WNV, Zika Virus, Chikungunya Virus, Dengue Virus, and Babesia.

K14. The method of K1, wherein the plurality of pathogens or infectious agents are HIV-1, HIV-2, HCV, HBV, WNV, Zika Virus, Chikungunya Virus, Dengue Virus, Babesia, and Malaria.

K15. The method of K1, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

K16. The method of K1, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

K17. The method of K1, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

K18. The method of K1, wherein the plurality of pathogens or infectious agents are HIV-1, HIV-2, HCV, HBV, and Parvovirus B19.

K19. The method of K1, wherein the plurality of pathogens or infectious agents are HIV-1, HIV-2, HCV, HBV, Parvovirus B19, and HAV.

K20. The method of K1, wherein the plurality of pathogens or infectious agents are HIV-1, HIV-2, HCV, HBV, and Babesia.

K21. The method of K1, wherein the plurality of pathogens or infectious agents are HIV-1, HIV-2, HCV, HBV, and HAV.

K22. The method of K1, wherein the plurality of pathogens or infectious agents are HIV-1, HIV-2, HCV, HBV, and HEV.

K23. The method of K1, wherein the plurality of pathogens or infectious agents are HIV-1, HIV-2, HCV, HBV, and Zika Virus.

K24. The method of K1, wherein the plurality of pathogens or infectious agents are HIV-1, HIV-2, HCV, HBV, Zika Virus, and Dengue Virus.

K25. The method of K1, wherein the plurality of pathogens or infectious agents are HIV-1, HIV-2, HCV, HBV, Zika Virus, Dengue Virus, and Chikungunya Virus.

K26. The method of K1, wherein the plurality of pathogens or infectious agents are HIV-1, HIV-2, HCV, HBV, Zika Virus, Dengue Virus, and WNV.

K27. The method of K1, wherein the plurality of pathogens or infectious agents are HIV-1, HIV-2, HCV, HBV, Zika Virus, WNV, and Chikungunya Virus.

K28. The method of K1, wherein the plurality of pathogens or infectious agents are HIV-1, HIV-2, HCV, HBV, Zika Virus, WNV, Dengue Virus, and Chikungunya Virus.

K29. The method of K1, wherein the plurality of pathogens or infectious agents are HIV-1, HIV-2, HCV, HBV, and Malaria.

K30. The method of K1, wherein the plurality of pathogens or infectious agents are HIV-1, HIV-2, HCV, HBV, Malaria, and Babesia.

K31. The method of K1, wherein the plurality of pathogens or infectious agents are HIV-1, HIV-2, HCV, HBV, and Dengue Virus.

K32. The method of K1, wherein the plurality of pathogens or infectious agents are HIV-1, HIV-2, HCV, HBV, Dengue Virus, and Chikungunya Virus.

K33. The method of K1, wherein the plurality of pathogens or infectious agents are HIV-1, HIV-2, HCV, HBV, Dengue Virus, WNV, and Chikungunya Virus.

K34. The method of K1, wherein the plurality of pathogens or infectious agents are HIV-1, HIV-2, HCV, HBV, and Chikungunya Virus.

K35. The method of K1, wherein the plurality of pathogens or infectious agents are HIV-1, HIV-2, HCV, HBV, Chikungunya Virus, and Zika Virus.

K36. The method of K1, wherein the plurality of pathogens or infectious agents are HIV-1, HIV-2, HCV, HBV, Chikungunya Virus, Zika Virus, and WNV.

K37. The method of K1, wherein the plurality of pathogens or infectious agents are HIV-1, HIV-2, HCV, HBV, WNV, and Dengue Virus.

K38. The method of K1, wherein the plurality of pathogens or infectious agents are HIV-1, HIV-2, HCV, HBV, Chikungunya Virus, Zika Virus, and WNV.

K39. The method of K1, wherein: the plurality of pathogens or infectious agents are HIV-1, HIV-2, HCV, and HBV; and the nucleic acid analysis comprises multiplex analysis of HIV-1 and HIV-2.

K40. The method of K1, wherein: the plurality of pathogens or infectious agents are HIV-1, HIV-2, HCV, and HBV; and the nucleic acid analysis comprises multiplex analysis of HIV-1, HIV-2, and HCV.

K41. The method of K1, wherein: the plurality of pathogens or infectious agents are HIV-1, HIV-2, HCV, and HBV; and the nucleic acid analysis comprises multiplex analysis of HIV-1, HIV-2, and HBV.

K42. The method of K1, wherein: the plurality of pathogens or infectious agents are HIV-1, HIV-2, HCV, and HBV; and the nucleic acid analysis comprises multiplex analysis of HCV, and HBV.

K43. The method of K1, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

K44. The method of K1, wherein: the plurality of pathogens or infectious agents are HIV-1, HIV-2, HCV, and HBV; and the nucleic acid analysis comprises multiplex analysis of HIV-1, HIV-2, HCV, and HBV.

K45. The method of K1, wherein: the plurality of pathogens or infectious agents comprise Zika Virus, WNV, Chikungunya Virus and Dengue Virus; the nucleic acid analysis comprises multiplex analysis of Zika Virus and WNV.

K46. The method of K1, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

K47. The method of K1, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

K48. The method of K1, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

K49. The method of K1, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

K50. The method of K1, wherein: the plurality of pathogens or infectious agents comprise Babesia and Malaria; and the nucleic acid analysis comprises multiplex analysis of Babesia and Malaria.

K51. The method of K1, wherein: the plurality of pathogens or infectious agents comprise Parvovirus B19 and HAV; and the nucleic acid analysis comprises multiplex analysis of Parvovirus B19 and HAV.

K52. The method of K1, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

K53. The method of K1, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

K54. The method of K1, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

K55. The method of K1, wherein the plurality of pathogens or infectious agents are Zika Virus and WNV; and wherein the predetermined levels are: at least 1-50 copies/mL of Zika Virus; and at least 1-50 copies/mL of WNV.

K56. The method of K1, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

K57. The method of K1, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

K58. The method of K1, wherein the plurality of pathogens or infectious agents are Babesia and Malaria; and wherein the predetermined levels are: at least 1-20 copies/mL of Babesia; and at least 1-50 copies/mL of Malaria.

K59. The method of K1, wherein the plurality of pathogens or infectious agents are Parvovirus B19 and HAV; and wherein the predetermined levels are: at least 1-40 IU/mL of Parvovirus B19; and at least 1-10 IU/mL of HAV.

K60. The method of K1, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

K61. The method of K1, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

K62. The method of K1, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

K63. The method of K1, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

K64. The method of K1, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

K65. The method of K1, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

K66. The method of K1, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

K67. The method of K1, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

K68. The method of K1, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

K69. The method of K1, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

K70. The method of K1, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

K71. The method of K1, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

K72. The method of K1, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

K73. The method of K1, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

K74. The method of K1, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

K75. The method of K1, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

K76. The method of K1, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

K77. The method of K1, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

K78. The method of K1, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

K79. The method of K1, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

K80. The method of K1, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

K81. The method of K1, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

K82. The method of K1, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

K83. The method of K1, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

K84. The method of K1, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

K85. The method of K1, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

K86. The method of K1, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

K87. The method of K1, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

K88. The method of K1, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

K89. The method of K1, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

K90. The method of K1, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

K91. The method of K1, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

K92. The method of K1, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

K93. The method of K1, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

K94. The method of K1, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

K95. The method of K1, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

K96. The method of K1, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

K97. The method of K1, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

K98. The method of K1, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

K99. The method of K1, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

K100. The method of K1, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

K101. The method of any one of K-K100, wherein the sample or biological sample is a sample of donor blood.

K102. The method of any one of K-K100, wherein the sample of donor blood is human donor blood.

K103. The method of any one of K-K100, wherein the sample of donor blood is whole blood.

K104. The method of any one of K-K100, wherein the sample of donor blood is lysed whole blood.

K105. The method of any one of K-K100, wherein the sample of donor blood is serum.

K106. The method of any one of K-K100, wherein the sample of donor blood is plasma.

K107. The method of K-K106, wherein the nucleic acid analysis comprises a nucleic acid amplification reaction.

K108. The method of K107, wherein the nucleic acid amplification reaction is an isothermal reaction.

K109. The method of K108, wherein the isothermal reaction is recombinase polymerase amplification.

K110. The method of K108, wherein the isothermal reaction is a nicking enzyme amplification reaction.

K111. The method of K-K110, wherein the nucleic acid analysis comprises optical detection of the presence of a nucleic acid derived from at least one of the plurality of pathogens or infectious agents.

K112. The method of K-K111, wherein the nucleic acid analysis comprises digital detection of the presence of a nucleic acid derived from at least one of the plurality of pathogens or infectious agents.

K113. The method of K-K112, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

K114. The method of K113, wherein the plurality of times comprises optically detecting the signal at a predetermined interval of about every 20 seconds or about every 30 seconds.

K115. The method of K113 and K114, wherein the optically detecting a fluorescent signal for a plurality of times occurs over a period of about 12 minutes.

L. The present disclosure provides a method for screening a plurality of samples to determine whether donor blood associated with the samples is acceptable for transfusion comprising: performing a nucleic acid analysis on the samples including a sample preparation process and an amplification process; determining a result from the nucleic acid analysis; and making a determination whether donor blood is acceptable for transfusion based at least in part on the nucleic acid analysis result wherein the nucleic acid analysis comprises a nucleic acid amplification reaction of about 8 minutes to about 20 minutes in duration.

L1. The method of L, wherein each result includes a determination of a predetermined level of nucleic acid from at least one of a plurality of pathogens or infectious agents.

L2. The method of L1, wherein the determination of a level equal to or greater than the predetermined level indicates that the donor material is not released for clinical use.

L3. The method of L1, wherein the determination of a level less than the predetermined level indicates that the donor material is released for clinical use.

L4. The method of any one of L1-L3, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, and HEV.

L5. The method of any one of L1-L3, wherein the plurality of pathogens or infectious agents are HIV-1, HIV-2, HCV, and HBV.

L6. The method of any one of L1-L3, wherein the plurality of pathogens or infectious agents are HIV-1, HIV-2, HCV, HBV, and WNV.

L7. The method of any one of L1-L3, wherein the plurality of pathogens or infectious agents are Zika Virus and WNV.

L8. The method of any one of L1-L3, wherein the plurality of pathogens or infectious agents are Chikungunya Virus and Dengue Virus.

L9. The method of any one of L1-L3, wherein the plurality of pathogens or infectious agents are Zika Virus, WNV, Chikungunya Virus and Dengue Virus.

L10. The method of any one of L1-L3, wherein the plurality of pathogens or infectious agents are Babesia and Malaria.

L11. The method of any one of L1-L3, wherein the plurality of pathogens or infectious agents are Parvovirus B19 and HAV.

L12. The method of any one of L1-L3, wherein the plurality of pathogens or infectious agents are HIV-1, HIV-2, HCV, HBV, WNV, and Zika Virus.

L13. The method of any one of L1-L3, wherein the plurality of pathogens or infectious agents are HIV-1, HIV-2, HCV, HBV, WNV, Zika Virus, and Chikungunya Virus.

L14. The method of any one of L1-L3, wherein the plurality of pathogens or infectious agents are HIV-1, HIV-2, HCV, HBV, WNV, Zika Virus, Chikungunya Virus, and Dengue Virus.

L15. The method of any one of L1-L3, wherein the plurality of pathogens or infectious agents are HIV-1, HIV-2, HCV, HBV, WNV, Zika Virus, Chikungunya Virus, Dengue Virus, and Babesia.

L16. The method of any one of L1-L3, wherein the plurality of pathogens or infectious agents are HIV-1, HIV-2, HCV, HBV, WNV, Zika Virus, Chikungunya Virus, Dengue Virus, Babesia, and Malaria.

L17. The method of any one of L1-L3, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

L18. The method of any one of L1-L3, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

L19. The method of any one of L1-L3, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

L20. The method of any one of L1-L3, wherein the plurality of pathogens or infectious agents are HIV-1, HIV-2, HCV, HBV, and Parvovirus B19.

L21. The method of any one of L1-L3, wherein the plurality of pathogens or infectious agents are HIV-1, HIV-2, HCV, HBV, Parvovirus B19, and HAV.

L22. The method of any one of L1-L3, wherein the plurality of pathogens or infectious agents are HIV-1, HIV-2, HCV, HBV, and Babesia.

L23. The method of any one of L1-L3, wherein the plurality of pathogens or infectious agents are HIV-1, HIV-2, HCV, HBV, and HAV.

L24. The method of any one of L1-L3, wherein the plurality of pathogens or infectious agents are HIV-1, HIV-2, HCV, HBV, and HEV.

L25. The method of any one of L1-L3, wherein the plurality of pathogens or infectious agents are HIV-1, HIV-2, HCV, HBV, and Zika Virus.

L26. The method of any one of L1-L3, wherein the plurality of pathogens or infectious agents are HIV-1, HIV-2, HCV, HBV, Zika Virus, and Dengue Virus.

L27. The method of any one of L1-L3, wherein the plurality of pathogens or infectious agents are HIV-1, HIV-2, HCV, HBV, Zika Virus, Dengue Virus, and Chikungunya Virus.

L28. The method of any one of L1-L3, wherein the plurality of pathogens or infectious agents are HIV-1, HIV-2, HCV, HBV, Zika Virus, Dengue Virus, and WNV.

L29. The method of any one of L1-L3, wherein the plurality of pathogens or infectious agents are HIV-1, HIV-2, HCV, HBV, Zika Virus, WNV, and Chikungunya Virus.

L30. The method of any one of L1-L3, wherein the plurality of pathogens or infectious agents are HIV-1, HIV-2, HCV, HBV, Zika Virus, WNV, Dengue Virus, and Chikungunya Virus.

L31. The method of any one of L1-L3, wherein the plurality of pathogens or infectious agents are HIV-1, HIV-2, HCV, HBV, and Malaria.

L32. The method of any one of L1-L3, wherein the plurality of pathogens or infectious agents are HIV-1, HIV-2, HCV, HBV, Malaria, and Babesia.

L33. The method of any one of L1-L3, wherein the plurality of pathogens or infectious agents are HIV-1, HIV-2, HCV, HBV, and Dengue Virus.

L34. The method of any one of L1-L3, wherein the plurality of pathogens or infectious agents are HIV-1, HIV-2, HCV, HBV, Dengue Virus, and Chikungunya Virus.

L35. The method of any one of L1-L3, wherein the plurality of pathogens or infectious agents are HIV-1, HIV-2, HCV, HBV, Dengue Virus, WNV, and Chikungunya Virus.

L36. The method of any one of L1-L3, wherein the plurality of pathogens or infectious agents are HIV-1, HIV-2, HCV, HBV, and Chikungunya Virus.

L37. The method of any one of L1-L3, wherein the plurality of pathogens or infectious agents are HIV-1, HIV-2, HCV, HBV, Chikungunya Virus, and Zika Virus.

L38. The method of any one of L1-L3, wherein the plurality of pathogens or infectious agents are HIV-1, HIV-2, HCV, HBV, Chikungunya Virus, Zika Virus, and WNV.

L39. The method of any one of L1-L3, wherein the plurality of pathogens or infectious agents are HIV-1, HIV-2, HCV, HBV, WNV, and Dengue Virus.

L40. The method of any one of L1-L3, wherein the plurality of pathogens or infectious agents are HIV-1, HIV-2, HCV, HBV, Chikungunya Virus, Zika Virus, and WNV.

L41. The method of any one of L1-L3, wherein: the plurality of pathogens or infectious agents are HIV-1, HIV-2, HCV, and HBV; and the nucleic acid analysis comprises multiplex analysis of HIV-1 and HIV-2.

L42. The method of any one of L1-L3, wherein: the plurality of pathogens or infectious agents are HIV-1, HIV-2, HCV, and HBV; and the nucleic acid analysis comprises multiplex analysis of HIV-1, HIV-2, and HCV.

L43. The method of any one of L1-L3, wherein: the plurality of pathogens or infectious agents are HIV-1, HIV-2, HCV, and HBV; and the nucleic acid analysis comprises multiplex analysis of HIV-1, HIV-2, and HBV.

L44. The method of any one of L1-L3, wherein: the plurality of pathogens or infectious agents are HIV-1, HIV-2, HCV, and HBV; and the nucleic acid analysis comprises multiplex analysis of HCV, and HBV.

L45. The method of any one of L1-L3, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

L46. The method of any one of L1-L3, wherein: the plurality of pathogens or infectious agents are HIV-1, HIV-2, HCV, and HBV; and the nucleic acid analysis comprises multiplex analysis of HIV-1, HIV-2, HCV, and HBV.

L47. The method of any one of L1-L3, wherein: the plurality of pathogens or infectious agents comprise Zika Virus, WNV, Chikungunya Virus and Dengue Virus; the nucleic acid analysis comprises multiplex analysis of Zika Virus and WNV.

L48. The method of any one of L1-L3, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

L49. The method of any one of L1-L3, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

L50. The method of any one of L1-L3, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

L51. The method of any one of L1-L3, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

L52. The method of any one of L1-L3, wherein: the plurality of pathogens or infectious agents comprise Babesia and Malaria; and the nucleic acid analysis comprises multiplex analysis of Babesia and Malaria.

L53. The method of any one of L1-L3, wherein: the plurality of pathogens or infectious agents comprise Parvovirus B19 and HAV; and the nucleic acid analysis comprises multiplex analysis of Parvovirus B19 and HAV.

L54. The method of any one of L1-L3, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

L55. The method of any one of L1-L3, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

L56. The method of any one of L1-L3, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

L57. The method of any one of L1-L3, wherein the plurality of pathogens or infectious agents are Zika Virus and WNV; and wherein the predetermined levels are: at least 1-50 copies/mL of Zika Virus; and at least 1-50 copies/mL of WNV.

L58. The method of any one of L1-L3, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

L59. The method of any one of L1-L3, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

L60. The method of any one of L1-L3, wherein the plurality of pathogens or infectious agents are Babesia and Malaria; and wherein the predetermined levels are: at least 1-20 copies/mL of Babesia; and at least 1-50 copies/mL of Malaria.

L61. The method of any one of L1-L3, wherein the plurality of pathogens or infectious agents are Parvovirus B19 and HAV; and wherein the predetermined levels are: at least 1-40 IU/mL of Parvovirus B19; and at least 1-10 IU/mL of HAV.

L62. The method of any one of L1-L3, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

L63. The method of any one of L1-L3, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

L64. The method of any one of L1-L3, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

L65. The method of any one of L1-L3, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

L66. The method of any one of L1-L3, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

L67. The method of any one of L1-L3, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

L68. The method of any one of L1-L3, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

L69. The method of any one of L1-L3, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

L70. The method of any one of L1-L3, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

L71. The method of any one of L1-L3, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

L72. The method of any one of L1-L3, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

L73. The method of any one of L1-L3, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

L74. The method of any one of L1-L3, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

L75. The method of any one of L1-L3, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

L76. The method of any one of L1-L3, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

L77. The method of any one of L1-L3, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

L78. The method of any one of L1-L3, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

L79. The method of any one of L1-L3, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

L80. The method of any one of L1-L3, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

L81. The method of any one of L1-L3, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

L82. The method of any one of L1-L3, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

L83. The method of any one of L1-L3, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

L84. The method of any one of L1-L3, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

L85. The method of any one of L1-L3, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

L86. The method of any one of L1-L3, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

L87. The method of any one of L1-L3, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

L88. The method of any one of L1-L3, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

L89. The method of any one of L1-L3, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

L90. The method of any one of L1-L3, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

L91. The method of any one of L1-L3, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

L92. The method of any one of L1-L3, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

L93. The method of any one of L1-L3, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

L94. The method of any one of L1-L3, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

L95. The method of any one of L1-L3, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

L96. The method of any one of L1-L3, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

L97. The method of any one of L1-L3, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

L98. The method of any one of L1-L3, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

L99. The method of any one of L1-L3, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

L100. The method of any one of L1-L3, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

L101. The method of any one of L1-L3, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

L102. The method of any one of L1-L3, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

L103. The method of any one of L-L102, wherein the sample or biological sample is a sample of donor blood.

L104. The method of any one of L-L102, wherein the sample of donor blood is human donor blood.

L105. The method of any one of L-L102, wherein the sample of donor blood is whole blood.

L106. The method of any one of L-L102, wherein the sample of donor blood is lysed whole blood.

L107. The method of any one of L-L102, wherein the sample of donor blood is serum.

L108. The method of any one of L-L102, wherein the sample of donor blood is plasma.

L109. The method of L-L108, wherein the nucleic acid analysis comprises a nucleic acid amplification reaction.

L110. The method of L109, wherein the nucleic acid amplification reaction is an isothermal reaction.

L111. The method of L110, wherein the isothermal reaction is recombinase polymerase amplification.

L112. The method of L110, wherein the isothermal reaction is a nicking enzyme amplification reaction.

L113. The method of L-L112, wherein the nucleic acid analysis comprises optical detection of the presence of a nucleic acid derived from at least one of the plurality of pathogens or infectious agents.

L114. The method of L-L113, wherein the nucleic acid analysis comprises digital detection of the presence of a nucleic acid derived from at least one of the plurality of pathogens or infectious agents.

L115. The method of L-L114, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

L116. The method of L115, wherein the plurality of times comprises optically detecting the signal at a predetermined interval of about every 20 seconds or about every 30 seconds.

L117. The method of L115 and L116, wherein the optically detecting a fluorescent signal for a plurality of times occurs over a period of about 12 minutes.

M. The present disclosure provides a method for screening a plurality of samples to determine whether donor blood associated with the samples is acceptable for transfusion comprising: performing a nucleic acid analysis on the samples including a sample preparation process and an amplification process; determining a result from the nucleic acid analysis; and making a determination whether donor blood is acceptable for transfusion based at least in part on the nucleic acid analysis result, wherein each result includes a determination of a predetermined level of nucleic acid from at least one of a plurality of pathogens or infectious agents, and wherein each determination is completed in about 20 to about 45 minutes from initial aspiration of the sample for performance of the nucleic acid analysis.

M1. The method of M, wherein the determination of a level equal to or greater than the predetermined level indicates that the donor material is not released for clinical use.

M2. The method of M, wherein the determination of a level less than the predetermined level indicates that the donor material is released for clinical use.

M3. The method of any one of M-M2, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu Virus and HEV.

M4. The method of any one of M-M2, wherein the plurality of pathogens or infectious agents are HIV-1, HIV-2, HCV, and HBV.

M5. The method of M, wherein the plurality of pathogens or infectious agents are HIV-1, HIV-2, HCV, HBV, and WNV.

M6. The method of M, wherein the plurality of pathogens or infectious agents are Zika Virus and WNV.

M7. The method of M, wherein the plurality of pathogens or infectious agents are Chikungunya Virus and Dengue Virus.

M8. The method of M, wherein the plurality of pathogens or infectious agents are Zika Virus, WNV, Chikungunya Virus and Dengue Virus.

M9. The method of M, wherein the plurality of pathogens or infectious agents are Babesia and Malaria.

M10. The method of M, wherein the plurality of pathogens or infectious agents are Parvovirus B19 and HAV.

M11. The method of M, wherein the plurality of pathogens or infectious agents are HIV-1, HIV-2, HCV, HBV, WNV, and Zika Virus.

M12. The method of M, wherein the plurality of pathogens or infectious agents are HIV-1, HIV-2, HCV, HBV, WNV, Zika Virus, and Chikungunya Virus.

M13. The method of M, wherein the plurality of pathogens or infectious agents are HIV-1, HIV-2, HCV, HBV, WNV, Zika Virus, Chikungunya Virus, and Dengue Virus.

M14. The method of M, wherein the plurality of pathogens or infectious agents are HIV-1, HIV-2, HCV, HBV, WNV, Zika Virus, Chikungunya Virus, Dengue Virus, and Babesia.

M15. The method of M, wherein the plurality of pathogens or infectious agents are HIV-1, HIV-2, HCV, HBV, WNV, Zika Virus, Chikungunya Virus, Dengue Virus, Babesia, and Malaria.

M16. The method of M, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

M17. The method of M, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

M18. The method of M, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

M19. The method of M, wherein the plurality of pathogens or infectious agents are HIV-1, HIV-2, HCV, HBV, and Parvovirus B19.

M20. The method of M, wherein the plurality of pathogens or infectious agents are HIV-1, HIV-2, HCV, HBV, Parvovirus B19, and HAV.

M21. The method of M, wherein the plurality of pathogens or infectious agents are HIV-1, HIV-2, HCV, HBV, and Babesia.

M22. The method of M, wherein the plurality of pathogens or infectious agents are HIV-1, HIV-2, HCV, HBV, and HAV.

M23. The method of M, wherein the plurality of pathogens or infectious agents are HIV-1, HIV-2, HCV, HBV, and HEV.

M24. The method of M, wherein the plurality of pathogens or infectious agents are HIV-1, HIV-2, HCV, HBV, and Zika Virus.

M25. The method of M, wherein the plurality of pathogens or infectious agents are HIV-1, HIV-2, HCV, HBV, Zika Virus, and Dengue Virus.

M26. The method of M, wherein the plurality of pathogens or infectious agents are HIV-1, HIV-2, HCV, HBV, Zika Virus, Dengue Virus, and Chikungunya Virus.

M27. The method of M, wherein the plurality of pathogens or infectious agents are HIV-1, HIV-2, HCV, HBV, Zika Virus, Dengue Virus, and WNV.

M28. The method of M, wherein the plurality of pathogens or infectious agents are HIV-1, HIV-2, HCV, HBV, Zika Virus, WNV, and Chikungunya Virus.

M29. The method of M, wherein the plurality of pathogens or infectious agents are HIV-1, HIV-2, HCV, HBV, Zika Virus, WNV, Dengue Virus, and Chikungunya Virus.

M30. The method of M, wherein the plurality of pathogens or infectious agents are HIV-1, HIV-2, HCV, HBV, and Malaria.

M31. The method of M, wherein the plurality of pathogens or infectious agents are HIV-1, HIV-2, HCV, HBV, Malaria, and Babesia.

M32. The method of M, wherein the plurality of pathogens or infectious agents are HIV-1, HIV-2, HCV, HBV, and Dengue Virus.

M33. The method of M, wherein the plurality of pathogens or infectious agents are HIV-1, HIV-2, HCV, HBV, Dengue Virus, and Chikungunya Virus.

M34. The method of M, wherein the plurality of pathogens or infectious agents are HIV-1, HIV-2, HCV, HBV, Dengue Virus, WNV, and Chikungunya Virus.

M35. The method of M, wherein the plurality of pathogens or infectious agents are HIV-1, HIV-2, HCV, HBV, and Chikungunya Virus.

M36. The method of M, wherein the plurality of pathogens or infectious agents are HIV-1, HIV-2, HCV, HBV, Chikungunya Virus, and Zika Virus.

M37. The method of M, wherein the plurality of pathogens or infectious agents are HIV-1, HIV-2, HCV, HBV, Chikungunya Virus, Zika Virus, and WNV.

M38. The method of M, wherein the plurality of pathogens or infectious agents are HIV-1, HIV-2, HCV, HBV, WNV, and Dengue Virus.

M39. The method of M, wherein the plurality of pathogens or infectious agents are HIV-1, HIV-2, HCV, HBV, Chikungunya Virus, Zika Virus, and WNV.

M40. The method of M, wherein: the plurality of pathogens or infectious agents are HIV-1, HIV-2, HCV, and HBV; and the nucleic acid analysis comprises multiplex analysis of HIV-1 and HIV-2.

M41. The method of M, wherein: the plurality of pathogens or infectious agents are HIV-1, HIV-2, HCV, and HBV; and the nucleic acid analysis comprises multiplex analysis of HIV-1, HIV-2, and HCV.

M42. The method of M, wherein: the plurality of pathogens or infectious agents are HIV-1, HIV-2, HCV, and HBV; and the nucleic acid analysis comprises multiplex analysis of HIV-1, HIV-2, and HBV.

M43. The method of M, wherein: the plurality of pathogens or infectious agents are HIV-1, HIV-2, HCV, and HBV; and the nucleic acid analysis comprises multiplex analysis of HCV, and HBV.

M44. The method of M, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

M45. The method of M, wherein: the plurality of pathogens or infectious agents are HIV-1, HIV-2, HCV, and HBV; and the nucleic acid analysis comprises multiplex analysis of HIV-1, HIV-2, HCV, and HBV.

M46. The method of M, wherein: the plurality of pathogens or infectious agents comprise Zika Virus, WNV, Chikungunya Virus and Dengue Virus; the nucleic acid analysis comprises multiplex analysis of Zika Virus and WNV.

M47. The method of M, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

M48. The method of M, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

M49. The method of M, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

M50. The method of M, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

M51. The method of M, wherein: the plurality of pathogens or infectious agents comprise Babesia and Malaria; and the nucleic acid analysis comprises multiplex analysis of Babesia and Malaria.

M52. The method of M, wherein: the plurality of pathogens or infectious agents comprise Parvovirus B19 and HAV; and the nucleic acid analysis comprises multiplex analysis of Parvovirus B19 and HAV.

M53. The method of M, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

M54. The method of M, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

M55. The method of M, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

M56. The method of M, wherein the plurality of pathogens or infectious agents are Zika Virus and WNV; and wherein the predetermined levels are: at least 1-50 copies/mL of Zika Virus; and at least 1-50 copies/mL of WNV.

M57. The method of M, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

M58. The method of M, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

M59. The method of M, wherein the plurality of pathogens or infectious agents are Babesia and Malaria; and wherein the predetermined levels are: at least 1-20 copies/mL of Babesia; and at least 1-50 copies/mL of Malaria.

M60. The method of M, wherein the plurality of pathogens or infectious agents are Parvovirus B19 and HAV; and wherein the predetermined levels are: at least 1-40 IU/mL of Parvovirus B19; and at least 1-10 IU/mL of HAV.

M61. The method of M, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

M62. The method of M, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

M63. The method of M, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

M64. The method of M, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

M65. The method of M, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

M66. The method of M, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

M67. The method of M, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

M68. The method of M, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

M69. The method of M, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

M70. The method of M, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

M71. The method of M, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

M72. The method of M, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

M73. The method of M, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

M74. The method of M, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

M75. The method of M, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

M76. The method of M, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

M77. The method of M, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

M78. The method of M, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

M79. The method of M, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

M80. The method of M, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

M81. The method of M, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

M82. The method of M, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

M83. The method of M, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

M84. The method of M, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

M85. The method of M, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

M86. The method of M, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

M87. The method of M, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

M88. The method of M, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

M89. The method of M, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

M90. The method of M, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

M91. The method of M, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

M92. The method of M, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

M93. The method of M, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

M94. The method of M, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

M95. The method of M, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

M96. The method of M, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

M97. The method of M, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

M98. The method of M, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

M99. The method of M, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

M100. The method of M, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

M101. The method of M, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

M102. The method of M, wherein the sample or biological sample is a sample of donor blood.

M103. The method of M102, wherein the sample of donor blood is human donor blood.

M104. The method of M102, wherein the sample of donor blood is whole blood.

M105. The method of M102, wherein the sample of donor blood is lysed whole blood.

M106. The method of M102, wherein the sample of donor blood is serum.

M107. The method of M102, wherein the sample of donor blood is plasma.

M108. The method of any one of M-M107, wherein the nucleic acid analysis comprises a nucleic acid amplification reaction.

M109. The method of M108, wherein the nucleic acid amplification reaction is an isothermal reaction.

M110. The method of M109, wherein the isothermal reaction is recombinase polymerase amplification.

MIll. The method of M109, wherein the isothermal reaction is a nicking enzyme amplification reaction.

M112. The method of any one of M-M111, wherein the nucleic acid analysis comprises optical detection of the presence of a nucleic acid derived from at least one of the plurality of pathogens or infectious agents.

M113. The method of any one of M-M112, wherein the nucleic acid analysis comprises digital detection of the presence of a nucleic acid derived from at least one of the plurality of pathogens or infectious agents.

M114. The method of any one of M-M113, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

M115. The method of M114, wherein the plurality of times comprises optically detecting the signal at a predetermined interval of about every 20 seconds or about every 30 seconds.

M116. The method of M114 and M115, wherein the optically detecting a fluorescent signal for a plurality of times occurs over a period of about 12 minutes.

N. The present disclosure provides a method for screening a plurality of samples to determine whether donor blood associated with the samples is acceptable for transfusion comprising: performing a nucleic acid analysis on the samples including a sample preparation process and an amplification process; determining a result from the nucleic acid analysis; and making a determination whether donor blood is acceptable for transfusion based at least in part on the nucleic acid analysis result, wherein each result includes a determination of a predetermined level of nucleic acid from at least one of a plurality of pathogens or infectious agents, and wherein each determination has a time to result of about 20 to about 45 minutes.

N1. The method of N, wherein the sample or biological sample is a sample of donor blood.

N2. The method of N1, wherein the sample of donor blood is human donor blood.

N3. The method of N1, wherein the sample of donor blood is whole blood.

N4. The method of N1, wherein the sample of donor blood is lysed whole blood.

N5. The method of N1, wherein the sample of donor blood is serum.

N6. The method of N1, wherein the sample of donor blood is plasma.

N7. The method of N-N6, wherein the nucleic acid analysis comprises a nucleic acid amplification reaction.

N8. The method of N7, wherein the nucleic acid amplification reaction is an isothermal reaction.

N9. The method of N8, wherein the isothermal reaction is recombinase polymerase amplification.

N10. The method of N8, wherein the isothermal reaction is a nicking enzyme amplification reaction.

N11. The method of any one of N-N10, wherein the nucleic acid analysis comprises optical detection of the presence of a nucleic acid derived from at least one of the plurality of pathogens or infectious agents.

N12. The method of any one of N-N11, wherein the nucleic acid analysis comprises digital detection of the presence of a nucleic acid derived from at least one of the plurality of pathogens or infectious agents.

N13. The method of any one of N-N12, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

N14. The method of N13, wherein the plurality of times comprises optically detecting the signal at a predetermined interval of about every 20 seconds or about every 30 seconds.

N15. The method of N13 and N14, wherein the optically detecting a fluorescent signal for a plurality of times occurs over a period of about 12 minutes.

O. The present disclosure provides a method for screening a plurality of samples to determine whether donor blood associated with the samples is acceptable for transfusion comprising: performing a nucleic acid analysis on the samples on an automated system, including a sample preparation process and an amplification process; determining a result from each of the nucleic acid analyses of the samples; and making a determination whether donor blood is acceptable for transfusion based at least in part on the nucleic acid analysis results wherein at least about 70 results are obtained per hour per m³ of a volume occupied by the automated system.

O1. The method of O, wherein each result includes a determination of the presence of absence of nucleic acid from at least one of a plurality of pathogens or infectious agents.

O1-1. The method of O, wherein each result includes a determination of a predetermined level of nucleic acid from at least one of a plurality of pathogens or infectious agents.

O2. The method of O1, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, Usutu and HEV.

O3. The method of O1, wherein the plurality of pathogens or infectious agents are HIV-1, HIV-2, HCV, and HBV.

O4. The method of O1, wherein the plurality of pathogens or infectious agents are HIV-1, HIV-2, HCV, HBV, and WNV.

O5. The method of O1, wherein the plurality of pathogens or infectious agents are Zika Virus and WNV.

O6. The method of O1, wherein the plurality of pathogens or infectious agents are Chikungunya Virus and Dengue Virus.

O7. The method of O1, wherein the plurality of pathogens or infectious agents are Zika Virus, WNV, Chikungunya Virus and Dengue Virus.

O8. The method of O1, wherein the plurality of pathogens or infectious agents are Babesia and Malaria.

O9. The method of O1, wherein the plurality of pathogens or infectious agents are Parvovirus B19 and HAV.

O10. The method of O1, wherein the plurality of pathogens or infectious agents are HIV-1, HIV-2, HCV, HBV, WNV, and Zika Virus.

O11. The method of O1, wherein the plurality of pathogens or infectious agents are HIV-1, HIV-2, HCV, HBV, WNV, Zika Virus, and Chikungunya Virus.

O12. The method of O1, wherein the plurality of pathogens or infectious agents are HIV-1, HIV-2, HCV, HBV, WNV, Zika Virus, Chikungunya Virus, and Dengue Virus.

O13. The method of O1, wherein the plurality of pathogens or infectious agents are HIV-1, HIV-2, HCV, HBV, WNV, Zika Virus, Chikungunya Virus, Dengue Virus, and Babesia.

O14. The method of O1, wherein the plurality of pathogens or infectious agents are HIV-1, HIV-2, HCV, HBV, WNV, Zika Virus, Chikungunya Virus, Dengue Virus, Babesia, and Malaria.

O15. The method of O1, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

O16. The method of O1, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

O17. The method of O1, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

O18. The method of O1, wherein the plurality of pathogens or infectious agents are HIV-1, HIV-2, HCV, HBV, and Parvovirus B19.

O19. The method of O1, wherein the plurality of pathogens or infectious agents are HIV-1, HIV-2, HCV, HBV, Parvovirus B19, and HAV.

O20. The method of O1, wherein the plurality of pathogens or infectious agents are HIV-1, HIV-2, HCV, HBV, and Babesia.

O21. The method of O1, wherein the plurality of pathogens or infectious agents are HIV-1, HIV-2, HCV, HBV, and HAV.

O22. The method of O1, wherein the plurality of pathogens or infectious agents are HIV-1, HIV-2, HCV, HBV, and HEV.

O23. The method of O1, wherein the plurality of pathogens or infectious agents are HIV-1, HIV-2, HCV, HBV, and Zika Virus.

O24. The method of O1, wherein the plurality of pathogens or infectious agents are HIV-1, HIV-2, HCV, HBV, Zika Virus, and Dengue Virus.

O25. The method of O1, wherein the plurality of pathogens or infectious agents are HIV-1, HIV-2, HCV, HBV, Zika Virus, Dengue Virus, and Chikungunya Virus.

O26. The method of O1, wherein the plurality of pathogens or infectious agents are HIV-1, HIV-2, HCV, HBV, Zika Virus, Dengue Virus, and WNV.

O27. The method of O1, wherein the plurality of pathogens or infectious agents are HIV-1, HIV-2, HCV, HBV, Zika Virus, WNV, and Chikungunya Virus.

O28. The method of O1, wherein the plurality of pathogens or infectious agents are HIV-1, HIV-2, HCV, HBV, Zika Virus, WNV, Dengue Virus, and Chikungunya Virus.

O29. The method of O1, wherein the plurality of pathogens or infectious agents are HIV-1, HIV-2, HCV, HBV, and Malaria.

O30. The method of O1, wherein the plurality of pathogens or infectious agents are HIV-1, HIV-2, HCV, HBV, Malaria, and Babesia.

O31. The method of O1, wherein the plurality of pathogens or infectious agents are HIV-1, HIV-2, HCV, HBV, and Dengue Virus.

O32. The method of O1, wherein the plurality of pathogens or infectious agents are HIV-1, HIV-2, HCV, HBV, Dengue Virus, and Chikungunya Virus.

O33. The method of O1, wherein the plurality of pathogens or infectious agents are HIV-1, HIV-2, HCV, HBV, Dengue Virus, WNV, and Chikungunya Virus.

O34. The method of O1, wherein the plurality of pathogens or infectious agents are HIV-1, HIV-2, HCV, HBV, and Chikungunya Virus.

O35. The method of O1, wherein the plurality of pathogens or infectious agents are HIV-1, HIV-2, HCV, HBV, Chikungunya Virus, and Zika Virus.

O36. The method of O1, wherein the plurality of pathogens or infectious agents are HIV-1, HIV-2, HCV, HBV, Chikungunya Virus, Zika Virus, and WNV.

O37. The method of O1, wherein the plurality of pathogens or infectious agents are HIV-1, HIV-2, HCV, HBV, WNV, and Dengue Virus.

O38. The method of O1, wherein the plurality of pathogens or infectious agents are HIV-1, HIV-2, HCV, HBV, Chikungunya Virus, Zika Virus, and WNV.

O39. The method of O1, wherein: the plurality of pathogens or infectious agents are HIV-1, HIV-2, HCV, and HBV; and the nucleic acid analysis comprises multiplex analysis of HIV-1 and HIV-2.

O40. The method of O1, wherein: the plurality of pathogens or infectious agents are HIV-1, HIV-2, HCV, and HBV; and the nucleic acid analysis comprises multiplex analysis of HIV-1, HIV-2, and HCV.

O41. The method of O1, wherein: the plurality of pathogens or infectious agents are HIV-1, HIV-2, HCV, and HBV; and the nucleic acid analysis comprises multiplex analysis of HIV-1, HIV-2, and HBV.

O42. The method of O1, wherein: the plurality of pathogens or infectious agents are HIV-1, HIV-2, HCV, and HBV; and the nucleic acid analysis comprises multiplex analysis of HCV, and HBV.

O43. The method of O1, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

O44. The method of O1, wherein: the plurality of pathogens or infectious agents are HIV-1, HIV-2, HCV, and HBV; and the nucleic acid analysis comprises multiplex analysis of HIV-1, HIV-2, HCV, and HBV.

O45. The method of O1, wherein: the plurality of pathogens or infectious agents comprise Zika Virus, WNV, Chikungunya Virus and Dengue Virus; the nucleic acid analysis comprises multiplex analysis of Zika Virus and WNV.

O46. The method of O1, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

O47. The method of O1, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

O48. The method of O1, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

O49. The method of O1, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

O50. The method of O1, wherein: the plurality of pathogens or infectious agents comprise Babesia and Malaria; and the nucleic acid analysis comprises multiplex analysis of Babesia and Malaria.

O51. The method of O1, wherein: the plurality of pathogens or infectious agents comprise Parvovirus B19 and HAV; and the nucleic acid analysis comprises multiplex analysis of Parvovirus B19 and HAV.

O52. The method of O1, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

O53. The method of O1, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

O54. The method of O1, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

O55. The method of O1, wherein the plurality of pathogens or infectious agents are Zika Virus and WNV; and wherein the predetermined levels are: at least 1-50 copies/mL of Zika Virus; and at least 1-50 copies/mL of WNV.

O56. The method of O1, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

O57. The method of O1, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

O58. The method of O1, wherein the plurality of pathogens or infectious agents are Babesia and Malaria; and wherein the predetermined levels are: at least 1-20 copies/mL of Babesia; and at least 1-50 copies/mL of Malaria.

O59. The method of O1, wherein the plurality of pathogens or infectious agents are Parvovirus B19 and HAV; and wherein the predetermined levels are: at least 1-40 IU/mL of Parvovirus B19; and at least 1-10 IU/mL of HAV.

O60. The method of O1, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

O61. The method of O1, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

O62. The method of O1, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

O63. The method of O1, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

O64. The method of O1, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

O65. The method of O1, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

O66. The method of O1, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

O67. The method of O1, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

O68. The method of O1, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

O69. The method of O1, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

O70. The method of O1, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

O71. The method of O1, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

O72. The method of O1, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

O73. The method of O1, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

O74. The method of O1, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

O75. The method of O1, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

O76. The method of O1, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

O77. The method of O1, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

O78. The method of O1, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

O79. The method of O1, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

O80. The method of O1, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

O81. The method of O1, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

O82. The method of O1, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

O83. The method of O1, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

O84. The method of O1, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

O85. The method of O1, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

O86. The method of O1, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

O87. The method of O1, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

O88. The method of O1, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

O89. The method of O1, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

O90. The method of O1, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

O91. The method of O1, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

O92. The method of O1, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

O93. The method of O1, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

O94. The method of O1, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

O95. The method of O1, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

O96. The method of O1, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

O97. The method of O1, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

O98. The method of O1, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

O99. The method of O1, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

O100. The method of O1, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

O101. The method of any one of claims O-O100, wherein the sample or biological sample is a sample of donor blood.

O102. The method of O101, wherein the sample of donor blood is human donor blood.

O103. The method of O101, wherein the sample of donor blood is whole blood.

O104. The method of O101, wherein the sample of donor blood is lysed whole blood.

O105. The method of O101, wherein the sample of donor blood is serum.

O106. The method of O101, wherein the sample of donor blood is plasma.

O107. The method of O-O106, wherein the nucleic acid analysis comprises a nucleic acid amplification reaction.

O108. The method of O107, wherein the nucleic acid amplification reaction is an isothermal reaction.

O109. The method of O108, wherein the isothermal reaction is recombinase polymerase amplification.

O110. The method of O108, wherein the isothermal reaction is a nicking enzyme amplification reaction.

O111. The method of O-O110, wherein the nucleic acid analysis comprises optical detection of the presence of a nucleic acid derived from at least one of the plurality of pathogens or infectious agents.

O112. The method of O-O111, wherein the nucleic acid analysis comprises digital detection of the presence of a nucleic acid derived from at least one of the plurality of pathogens or infectious agents.

O113. The method of O-O112, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

O113-1. The method of O111, wherein the optical detection comprises optically detecting the signal at a predetermined interval a plurality of times during the amplification reaction.

O114. The method of O113 or 0113-1, wherein the plurality of times comprises optically detecting the signal at a predetermined interval of about every 20 seconds or about every 30 seconds.

O115. The method of O113, 0113-1 and 0114, wherein the optically detecting a fluorescent signal for a plurality of times occurs over a period of about 12 minutes.

O116. The method of O113, 0113-1 and 0114, wherein the optically detecting a fluorescent signal for a plurality of times occurs over a period of about 20 minutes.

O117. The method of O113, 0113-1 and 0114, wherein the optically detecting a fluorescent signal for a plurality of times occurs over a period of about 1 minute.

O118. The method of O113, 0113-1 and 0114, wherein the optically detecting a fluorescent signal for a plurality of times occurs over a period of about 5 minutes.

P. The present disclosure provides a method for screening a plurality of samples to determine whether donor blood associated with the samples is acceptable for transfusion comprising: performing a nucleic acid analysis on the samples on an automated system, including a sample preparation process and an amplification process; determining a result from each of the nucleic acid analyses of the samples; and making a determination whether donor blood is acceptable for transfusion based at least in part on the nucleic acid analysis result wherein at least about 140 results are obtained per hour per m² of a footprint of the automated system.

P1. The method of P, wherein each result comprises the presence or absence of a nucleic acid derived from at least one of the plurality of pathogens or infectious agents.

P1-1. The method of P, wherein each result includes a determination of a predetermined level of nucleic acid from at least one of a plurality of pathogens or infectious agents.

P2. The method of any one of P-P1-1, wherein the sample or biological sample is a sample of donor blood.

P3. The method of P2, wherein the sample of donor blood is human donor blood.

P4. The method of P2, wherein the sample of donor blood is whole blood.

P5. The method of P2, wherein the sample of donor blood is lysed whole blood.

P6. The method of P2, wherein the sample of donor blood is serum.

P7. The method of P2, wherein the sample of donor blood is plasma.

P8. The method of P-P7, wherein the nucleic acid analysis comprises a nucleic acid amplification reaction.

P9. The method of P8, wherein the nucleic acid amplification reaction is an isothermal reaction.

P10. The method of P9, wherein the isothermal reaction is recombinase polymerase amplification.

P11. The method of P9, wherein the isothermal reaction is a nicking enzyme amplification reaction.

P12. The method of P-P11, wherein the nucleic acid analysis comprises optical detection of the presence of a nucleic acid derived from at least one of the plurality of pathogens or infectious agents.

P13. The method of P-P12, wherein the nucleic acid analysis comprises digital detection of the presence of a nucleic acid derived from at least one of the plurality of pathogens or infectious agents.

P14. The method of P-P13, wherein the optical detection comprises optically detecting the signal at a predetermined interval a plurality of times during the amplification reaction.

P14-1. The method of P-P13, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

P15. The method of P14 or P14-1, wherein the plurality of times comprises optically detecting the signal at a predetermined interval of about every 20 seconds or about every 30 seconds.

P16. The method of P14, P14-1 and P15, wherein the optically detecting a fluorescent signal for a plurality of times occurs over a period of about 12 minutes.

P17. The method of P14, P14-1 and P15, wherein the optically detecting a fluorescent signal for a plurality of times occurs over a period of about 20 minutes.

P18. The method of P14, P14-1 and P15, wherein the optically detecting a fluorescent signal for a plurality of times occurs over a period of about 1 minute.

P19. The method of P14, P14-1 and P15, wherein the optically detecting a fluorescent signal for a plurality of times occurs over a period of about 5 minutes.

Q. The present disclosure provides for a method for the detection of a target nucleic acid derived from a pathogen or infectious agent in a sample, comprising: performing a nucleic acid analysis on an automated system, comprising: a) a sample preparation process including preparing nucleic acid from the sample for an amplification reaction; b) an amplification process including amplifying the target nucleic acid with the amplification reaction; and c) a detection process including detecting the presence or absence of the target nucleic acid amplified in the reaction, wherein the sample preparation process, amplification process and detection process are completed in less than about 60 minutes from initial aspiration of the sample for the nucleic acid analysis.

Q1. The method of Q, wherein the method includes detection of a target nucleic acid derived from a pathogen or infectious agent in a plurality of samples and wherein at least about 140 results are obtained per hour per m² of a footprint of the automated system.

Q2. The method of any one of Q-Q1, wherein between about 140 results and 300 results are obtained per hour per m² of a footprint of the automated system.

Q3. The method of any one of Q-Q2, wherein at least about 70 results are obtained per hour per m³ of a volume occupied by the automated system.

Q4. The method of any one of Q-Q3, wherein detecting the presence or absence of the target nucleic acid comprises optically detecting a fluorescent signal corresponding to the target nucleic acid at a plurality of times during the amplification reaction.

Q5. The method of any one of Q-Q3, wherein detecting the presence or absence of the target nucleic acid comprises determining an amount of the target nucleic acid.

Q6. The method of Q5, wherein determining the amount comprises optically detecting a fluorescent signal corresponding to the amount of the target nucleic acid at a plurality of times during the amplification reaction.

Q7. The method of any one of Q4 and Q6, wherein the plurality of times comprises optically detecting the signal at a predetermined interval of about every 12 second, about every 20 seconds, about every 30 seconds or about every 48 seconds.

Q8. The method of any one of Q4, Q6, and Q7, wherein the optically detecting a fluorescent signal for a plurality of times occurs over a period of about 12 minutes.

Q9. The method of any one of Q-Q8, wherein the method is for screening a sample for release of a donor material for clinical use.

Q10. The method of any one of Q-Q9, wherein the sample is a sample associated with donor blood.

Q11. The method of any one of Q-Q10, wherein the sample preparation process, amplification process and detection process are completed in about 15 minutes to about 60 minutes.

Q12. The method of any one of Q-Q11, wherein the sample preparation process, amplification process and detection process are completed in about 20 minutes to about 60 minutes.

Q13. The method of any one of Q-Q12, wherein the target nucleic acid comprises nucleic acid derived from at least one of SARS-CoV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, Usutu Virus and HEV.

Q14. The method of any one of Q-Q13, wherein the target nucleic acid comprises nucleic acid from a plurality of pathogens or infectious agents.

Q15. The method of Q14, wherein the plurality of pathogens or infectious agents are selected from the group consisting of: SARS-CoV-2 (COVID-19), HIV-1, HIV-2, HBV, HCV, CMV, Parvovirus B19, HAV, Chlamydia, Gonorrhea, WNV, Zika Virus, Dengue Virus, Chikungunya Virus, Influenza, Babesia, Malaria, Usutu Virus and HEV.

Q16. The method of Q9, wherein release of the donor material for clinical use occurs in about 15 to about 60 minutes from initial aspiration of the sample for performance of the nucleic acid analysis.

Q17. The method of Q9, wherein release of the donor material for clinical use occurs in about 20 to about 45 minutes from initial aspiration of the sample for performance of the nucleic acid analysis.

Q18. The method of Q-Q17, wherein the amplification process and the detection process is a combined nucleic acid amplification and detection process, wherein the nucleic acid amplification and detection process has a duration of about 1 minute to about 20 minutes.

Q19. The method of Q-Q18, wherein detecting the presence or absence of the target nucleic acid comprises a determination of a predetermined level of nucleic acid derived from one or more of a plurality of pathogens or infectious agents.

Q20. The method of any one of Q14-Q21, wherein the determination of a level equal to or greater than the predetermined level indicates that a donor material, donor blood, a biological sample, or donor plasma is not released for clinical use.

Q21. The method of any one of Q14-Q21, wherein the determination of a level less than the predetermined level indicates that ta donor material, a donor blood, a biological sample, or a plasma is released for clinical use.

Q22. The method of any one of Q14-Q21, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, and HEV.

Q23. The method of any one of Q14-Q21, wherein the plurality of pathogens or infectious agents are HIV-1, HIV-2, HCV, and HBV.

Q24. The method of any one of Q14-Q21, wherein the plurality of pathogens or infectious agents are HIV-1, HIV-2, HCV, HBV, and WNV.

Q25. The method of any one of Q14-Q21, wherein the plurality of pathogens or infectious agents are Zika Virus and WNV.

Q26. The method of any one of Q14-Q21, wherein the plurality of pathogens or infectious agents are Chikungunya Virus and Dengue Virus.

Q27. The method of any one of Q14-Q21, wherein the plurality of pathogens or infectious agents are Zika Virus, WNV, Chikungunya Virus and Dengue Virus.

Q28. The method of any one of Q14-Q21, wherein the plurality of pathogens or infectious agents are Babesia and Malaria.

Q29. The method of any one of Q14-Q21, wherein the plurality of pathogens or infectious agents are Parvovirus B19 and HAV.

Q30. The method of any one of Q14-Q21, wherein the plurality of pathogens or infectious agents are HIV-1, HIV-2, HCV, HBV, WNV, and Zika Virus.

Q31. The method of any one of Q14-Q21, wherein the plurality of pathogens or infectious agents are HIV-1, HIV-2, HCV, HBV, WNV, Zika Virus, and Chikungunya Virus.

Q32. The method of any one of Q14-Q21, wherein the plurality of pathogens or infectious agents are HIV-1, HIV-2, HCV, HBV, WNV, Zika Virus, Chikungunya Virus, and Dengue Virus.

Q33. The method of any one of Q14-Q21, wherein the plurality of pathogens or infectious agents are HIV-1, HIV-2, HCV, HBV, WNV, Zika Virus, Chikungunya Virus, Dengue Virus, and Babesia.

Q34. The method of any one of Q14-Q21, wherein the plurality of pathogens or infectious agents are HIV-1, HIV-2, HCV, HBV, WNV, Zika Virus, Chikungunya Virus, Dengue Virus, Babesia, and Malaria.

Q35. The method of any one of Q14-Q21, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

Q36. The method of any one of Q14-Q21, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

Q37. The method of any one of Q14-Q21, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

Q38. The method of any one of Q14-Q21, wherein the plurality of pathogens or infectious agents are HIV-1, HIV-2, HCV, HBV, and Parvovirus B19.

Q39. The method of any one of Q14-Q21, wherein the plurality of pathogens or infectious agents are HIV-1, HIV-2, HCV, HBV, Parvovirus B19, and HAV.

Q40. The method of any one of Q14-Q21, wherein the plurality of pathogens or infectious agents are HIV-1, HIV-2, HCV, HBV, and Babesia.

Q41. The method of any one of Q14-Q21, wherein the plurality of pathogens or infectious agents are HIV-1, HIV-2, HCV, HBV, and HAV.

Q42. The method of any one of Q14-Q21, wherein the plurality of pathogens or infectious agents are HIV-1, HIV-2, HCV, HBV, and HEV.

Q43. The method of any one of Q14-Q21, wherein the plurality of pathogens or infectious agents are HIV-1, HIV-2, HCV, HBV, and Zika Virus.

Q44. The method of any one of Q14-Q21, wherein the plurality of pathogens or infectious agents are HIV-1, HIV-2, HCV, HBV, Zika Virus, and Dengue Virus.

Q45. The method of any one of Q14-Q21, wherein the plurality of pathogens or infectious agents are HIV-1, HIV-2, HCV, HBV, Zika Virus, Dengue Virus, and Chikungunya Virus.

Q46. The method of any one of Q14-Q21, wherein the plurality of pathogens or infectious agents are HIV-1, HIV-2, HCV, HBV, Zika Virus, Dengue Virus, and WNV.

Q47. The method of any one of Q14-Q21, wherein the plurality of pathogens or infectious agents are HIV-1, HIV-2, HCV, HBV, Zika Virus, WNV, and Chikungunya Virus.

Q48. The method of any one of Q14-Q21, wherein the plurality of pathogens or infectious agents are HIV-1, HIV-2, HCV, HBV, Zika Virus, WNV, Dengue Virus, and Chikungunya Virus.

Q49. The method of any one of Q14-Q21, wherein the plurality of pathogens or infectious agents are HIV-1, HIV-2, HCV, HBV, and Malaria.

Q50. The method of any one of Q14-Q21, wherein the plurality of pathogens or infectious agents are HIV-1, HIV-2, HCV, HBV, Malaria, and Babesia.

Q51. The method of any one of Q14-Q21, wherein the plurality of pathogens or infectious agents are HIV-1, HIV-2, HCV, HBV, and Dengue Virus.

Q52. The method of any one of Q14-Q21, wherein the plurality of pathogens or infectious agents are HIV-1, HIV-2, HCV, HBV, Dengue Virus, and Chikungunya Virus.

Q53. The method of any one of Q14-Q21, wherein the plurality of pathogens or infectious agents are HIV-1, HIV-2, HCV, HBV, Dengue Virus, WNV, and Chikungunya Virus.

Q54. The method of any one of Q14-Q21, wherein the plurality of pathogens or infectious agents are HIV-1, HIV-2, HCV, HBV, and Chikungunya Virus.

Q55. The method of any one of Q14-Q21, wherein the plurality of pathogens or infectious agents are HIV-1, HIV-2, HCV, HBV, Chikungunya Virus, and Zika Virus.

Q56. The method of any one of Q14-Q21, wherein the plurality of pathogens or infectious agents are HIV-1, HIV-2, HCV, HBV, Chikungunya Virus, Zika Virus, and WNV.

Q57. The method of any one of Q14-Q21, wherein the plurality of pathogens or infectious agents are HIV-1, HIV-2, HCV, HBV, WNV, and Dengue Virus.

Q58. The method of any one of Q14-Q21, wherein the plurality of pathogens or infectious agents are HIV-1, HIV-2, HCV, HBV, Chikungunya Virus, Zika Virus, and WNV.

Q59. The method of any one of Q14-Q21, wherein: the plurality of pathogens or infectious agents are HIV-1, HIV-2, HCV, and HBV; and the nucleic acid analysis comprises multiplex analysis of HIV-1 and HIV-2.

Q60. The method of any one of Q14-Q21, wherein: the plurality of pathogens or infectious agents are HIV-1, HIV-2, HCV, and HBV; and the nucleic acid analysis comprises multiplex analysis of HIV-1, HIV-2, and HCV.

Q61. The method of any one of Q14-Q21, wherein: the plurality of pathogens or infectious agents are HIV-1, HIV-2, HCV, and HBV; and the nucleic acid analysis comprises multiplex analysis of HIV-1, HIV-2, and HBV.

Q62. The method of any one of Q14-Q21, wherein: the plurality of pathogens or infectious agents are HIV-1, HIV-2, HCV, and HBV; and the nucleic acid analysis comprises multiplex analysis of HCV, and HBV.

Q63. The method of any one of Q14-Q21, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

Q64. The method of any one of Q14-Q21, wherein: the plurality of pathogens or infectious agents are HIV-1, HIV-2, HCV, and HBV; and the nucleic acid analysis comprises multiplex analysis of HIV-1, HIV-2, HCV, and HBV.

Q65. The method of any one of Q14-Q21, wherein: the plurality of pathogens or infectious agents comprise Zika Virus, WNV, Chikungunya Virus and Dengue Virus; the nucleic acid analysis comprises multiplex analysis of Zika Virus and WNV.

Q66. The method of any one of Q14-Q21, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

Q67. The method of any one of Q14-Q21, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

Q68. The method of any one of Q14-Q21, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

Q69. The method of any one of Q14-Q21, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

Q70. The method of any one of Q14-Q21, wherein: the plurality of pathogens or infectious agents comprise Babesia and Malaria; and the nucleic acid analysis comprises multiplex analysis of Babesia and Malaria.

Q71. The method of any one of Q14-Q21, wherein: the plurality of pathogens or infectious agents comprise Parvovirus B19 and HAV; and the nucleic acid analysis comprises multiplex analysis of Parvovirus B19 and HAV.

Q72. The method of any one of Q14-Q21, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

Q73. The method of any one of Q14-Q21, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

Q74. The method of any one of Q14-Q21, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

Q75. The method of any one of Q14-Q21, wherein the plurality of pathogens or infectious agents are Zika Virus and WNV; and wherein the predetermined levels are: at least 1-50 copies/mL of Zika Virus; and at least 1-50 copies/mL of WNV.

Q76. The method of any one of Q14-Q21, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

Q77. The method of any one of Q14-Q21, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

Q78. The method of any one of Q14-Q21, wherein the plurality of pathogens or infectious agents are Babesia and Malaria; and wherein the predetermined levels are: at least 1-20 copies/mL of Babesia; and at least 1-50 copies/mL of Malaria.

Q79. The method of any one of Q14-Q21, wherein the plurality of pathogens or infectious agents are Parvovirus B19 and HAV; and wherein the predetermined levels are: at least 1-40 IU/mL of Parvovirus B19; and at least 1-10 IU/mL of HAV.

Q80. The method of any one of Q14-Q21, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

Q81. The method of any one of Q14-Q21, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

Q82. The method of any one of Q14-Q21, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

Q83. The method of any one of Q14-Q21, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

Q84. The method of any one of Q14-Q21, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

Q85. The method of any one of Q14-Q21, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

Q86. The method of any one of Q14-Q21, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

Q87. The method of any one of Q14-Q21, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

Q88. The method of any one of Q14-Q21, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

Q89. The method of any one of Q14-Q21, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

Q90. The method of any one of Q14-Q21, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

Q91. The method of any one of Q14-Q21, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

Q92. The method of any one of Q14-Q21, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

Q93. The method of any one of Q14-Q21, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

Q94. The method of any one of Q14-Q21, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

Q95. The method of any one of Q14-Q21, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

Q96. The method of any one of Q14-Q21, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

Q97. The method of any one of Q14-Q21, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

Q98. The method of any one of Q14-Q21, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

Q99. The method of any one of Q14-Q21, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

Q100. The method of any one of Q14-Q21, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

Q101. The method of any one of Q14-Q21, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

Q102. The method of any one of Q14-Q21, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

Q103. The method of any one of Q14-Q21, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

Q104. The method of any one of Q14-Q21, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

Q105. The method of any one of Q14-Q21, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

Q106. The method of any one of Q14-Q21, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

Q107. The method of any one of Q14-Q21, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

Q108. The method of any one of Q14-Q21, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

Q109. The method of any one of Q14-Q21, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

Q110. The method of any one of Q14-Q21, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

Q111. The method of any one of Q14-Q21, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

Q112. The method of any one of Q14-Q21, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

Q113. The method of any one of Q14-Q21, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

Q114. The method of any one of Q14-Q21, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

Q115. The method of any one of Q14-Q21, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

Q116. The method of any one of Q14-Q21, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

Q117. The method of any one of Q14-Q21, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

Q118. The method of any one of Q14-Q21, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

Q119. The method of any one of Q14-Q21, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

Q120. The method of any one of Q14-Q21, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

Q121. The method of Q-Q120, wherein the sample or biological sample is a sample of donor blood.

Q122. The method of Q121, wherein the sample of donor blood is human donor blood.

Q123. The method of Q121, wherein the sample of donor blood is whole blood.

Q124. The method of Q121, wherein the sample of donor blood is lysed whole blood.

Q125. The method of Q121, wherein the sample of donor blood is serum.

Q126. The method of Q121, wherein the sample of donor blood is plasma.

Q127. The method of any one of Q19 and Q22-Q126, wherein the release of the donor material is for transfusion.

Q128. The method of any one of Q19 and Q22-Q126, wherein the release of the donor material is for use in a pharmaceutical.

Q129. The method of any one of Q19 and Q22-Q126, wherein the release of the donor material is for use in a therapeutic treatment.

Q130. The method of any one of Q6, Q11-Q19 and Q22-Q126, wherein the clinical use is an in vivo clinical use.

Q131. The method of Q6, Q11-Q19 and Q22-Q126, wherein the clinical use is an in vitro clinical use.

Q132. The method of Q6, Q11-Q19, Q22-Q126 and Q130, wherein the clinical use is transfusion.

Q133. The method of Q6, Q11-Q19, Q22-Q126 and Q130, wherein the clinical use is use in a pharmaceutical.

Q134. The method of any one of Q6, Q11-Q19, Q22-Q126 and Q130, wherein the clinical use is use in a therapeutic treatment.

Q135. The method of any one of Q6, Q11-Q19, Q22-Q126 and Q130, wherein the clinical use is organ or tissue donation.

Q136. The method of any one of Q6, Q11-Q19, Q22-Q126 and Q130, wherein the clinical use is use in a plasma-derived product.

Q137. The method of any one of Q6, Q11-Q19, Q22-Q126 and Q131, wherein the clinical use is use in disease diagnostics or in quality assurance/laboratory diagnostics.

Q138. The method of any one of Q-Q137, wherein the nucleic acid analysis comprises a nucleic acid amplification reaction.

Q139. The method of Q138, wherein the nucleic acid amplification reaction is an isothermal reaction.

Q140. The method of Q139, wherein the isothermal reaction is recombinase polymerase amplification.

Q141. The method of Q139, wherein the isothermal reaction is anicking enzyme amplification reaction.

Q142. The method of any one of Q-Q141, wherein the nucleic acid analysis comprises optical detection of the presence of a nucleic acid derived from at least one of the plurality of pathogens or infectious agents.

Q143. The method of any one of Q-Q142, wherein the nucleic acid analysis comprises digital detection of the presence of a nucleic acid derived from at least one of the plurality of pathogens or infectious agents.

Q144. The method of Q143, wherein the optical detection comprises detecting a fluorescent signal corresponding to a target nucleic acid at a plurality of times during the amplification reaction.

Q144. The method of any one of Q-Q143, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

Q145. The method of any one of Q144 or Q144-1, wherein the plurality of times comprises optically detecting the signal at a predetermined interval of about every 12 seconds, about every 20 seconds, about every 30 seconds or about every 48 seconds.

Q146. The method of any one of Q144, Q144-1 and Q145, wherein the optically detecting a fluorescent signal for a plurality of times occurs over a period of about 12 minutes.

Q147. The method of any one of Q144, Q144-1 and Q145, wherein the optically detecting a fluorescent signal for a plurality of times occurs over a period of about 1 minute.

Q148. The method of any one of Q144, Q144-1 and Q145, wherein the optically detecting a fluorescent signal for a plurality of times occurs over a period of about 5 minutes.

Q146. The method of any one of Q144, Q144-1 and Q145, wherein the optically detecting a fluorescent signal for a plurality of times occurs over a period of about 20 minutes.

Q147. The method of any one of Q-Q146, wherein the amplification process and the detection process is a combined amplification and detection process.

R. The present disclosure provides a method for generating a plasma-derived product, comprising: a) performing a nucleic acid analysis on a donor plasma sample including a sample preparation process and an amplification process; b) determining a result from the nucleic acid analysis; and c) making a determination whether plasma associated with the donor plasma sample is acceptable for generating a plasma-derived product based at least in part on the nucleic acid analysis result, wherein release of the plasma for generating a plasma-derived product occurs in about 15 to about 60 minutes from initial aspiration of the donor plasma sample for performance of the nucleic acid analysis.

R1. The method of R, wherein release of the plasma for clinical use occurs in about 20 to about 60 minutes from initial aspiration of the donor plasma sample for performance of the nucleic acid analysis.

R2. The method of R, wherein release of the plasma for clinical use occurs in about 20 to about 45 minutes from initial aspiration of the donor plasma sample for performance of the nucleic acid analysis.

R3. The method of any one of R-R2, wherein the nucleic acid analysis comprises a nucleic acid amplification and detection process of about 1 minutes to about 20 minutes in duration.

R4. The method of any one of R-R3, wherein each result from the nucleic acid analysis has a time to result of about 20 to about 45 minutes.

R5. The method of any one of R-R4, wherein the nucleic acid analysis is performed on an automated system, and wherein at least about 70 results are obtained per hour per m³ of a volume occupied by the automated system.

R6. The method of any one of R-R5, wherein the nucleic acid analysis is performed on an automated system, and wherein at least about 140 results are obtained per hour per m² of a footprint of the automated system.

R7. The method of any one of R-R6, wherein each result includes a determination of the presence or absence of a nucleic acid from at least one of a plurality of pathogens or infectious agents.

R7-1. The method of any one of R-R6, wherein each result includes a determination of a predetermined level of nucleic acid from at least one of a plurality of pathogens or infectious agents.

R8. The method of R7 or R7-1, wherein the plurality of pathogens or infectious agents are selected from the group consisting of: HIV-1, HIV-2, HBV, HCV, CMV, Parvovirus B19, HAV, Gonorrhea, WNV, Zika Virus, Dengue Virus, Chikungunya Virus, Babesia, Malaria, HEV, Usutu Virus and a combination thereof.

R9. The method of any one of R-R8, wherein the plasma-derived product is selected from the group consisting of a coagulation factor, a protease inhibitor, albumin, immunoglobulin G (IgG) and a combination thereof.

R10. The method of R7-1, wherein the determination of a level equal to or greater than the predetermined level indicates that the plasma is not released for clinical use.

R11. The method of R7-1, wherein the determination of a level less than the predetermined level indicates that the plasma is released for clinical use.

R12. The method of any one of R8-R12, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, and HEV.

R13. The method of R8, wherein the plurality of pathogens or infectious agents are HIV-1, HIV-2, HCV, and HBV.

R14. The method of R8, wherein the plurality of pathogens or infectious agents are HIV-1, HIV-2, HCV, HBV, and WNV.

R15. The method of R8, wherein the plurality of pathogens or infectious agents are Zika Virus and WNV.

R16. The method of R8, wherein the plurality of pathogens or infectious agents are Chikungunya Virus and Dengue Virus.

R17. The method of R8, wherein the plurality of pathogens or infectious agents are Zika Virus, WNV, Chikungunya Virus and Dengue Virus.

R18. The method of R8, wherein the plurality of pathogens or infectious agents are Babesia and Malaria.

R19. The method of R8, wherein the plurality of pathogens or infectious agents are Parvovirus B19 and HAV.

R20. The method of R8, wherein the plurality of pathogens or infectious agents are HIV-1, HIV-2, HCV, HBV, WNV, and Zika Virus.

R21. The method of R8, wherein the plurality of pathogens or infectious agents are HIV-1, HIV-2, HCV, HBV, WNV, Zika Virus, and Chikungunya Virus.

R22. The method of R8, wherein the plurality of pathogens or infectious agents are HIV-1, HIV-2, HCV, HBV, WNV, Zika Virus, Chikungunya Virus, and Dengue Virus.

R23. The method of R8, wherein the plurality of pathogens or infectious agents are HIV-1, HIV-2, HCV, HBV, WNV, Zika Virus, Chikungunya Virus, Dengue Virus, and Babesia.

R24. The method of R8, wherein the plurality of pathogens or infectious agents are HIV-1, HIV-2, HCV, HBV, WNV, Zika Virus, Chikungunya Virus, Dengue Virus, Babesia, and Malaria.

R25. The method of R8, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

R26. The method of R8, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

R27. The method of R8, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

R28. The method of R8, wherein the plurality of pathogens or infectious agents are HIV-1, HIV-2, HCV, HBV, and Parvovirus B19.

R29. The method of R8, wherein the plurality of pathogens or infectious agents are HIV-1, HIV-2, HCV, HBV, Parvovirus B19, and HAV.

R30. The method of R8, wherein the plurality of pathogens or infectious agents are HIV-1, HIV-2, HCV, HBV, and Babesia.

R31. The method of R8, wherein the plurality of pathogens or infectious agents are HIV-1, HIV-2, HCV, HBV, and HAV.

R32. The method of R8, wherein the plurality of pathogens or infectious agents are HIV-1, HIV-2, HCV, HBV, and HEV.

R33. The method of R8, wherein the plurality of pathogens or infectious agents are HIV-1, HIV-2, HCV, HBV, and Zika Virus.

R34. The method of R8, wherein the plurality of pathogens or infectious agents are HIV-1, HIV-2, HCV, HBV, Zika Virus, and Dengue Virus.

R35. The method of R8, wherein the plurality of pathogens or infectious agents are HIV-1, HIV-2, HCV, HBV, Zika Virus, Dengue Virus, and Chikungunya Virus.

R36. The method of R8, wherein the plurality of pathogens or infectious agents are HIV-1, HIV-2, HCV, HBV, Zika Virus, Dengue Virus, and WNV.

R37. The method of R8, wherein the plurality of pathogens or infectious agents are HIV-1, HIV-2, HCV, HBV, Zika Virus, WNV, and Chikungunya Virus.

R38. The method of R8, wherein the plurality of pathogens or infectious agents are HIV-1, HIV-2, HCV, HBV, Zika Virus, WNV, Dengue Virus, and Chikungunya Virus.

R39. The method of R8, wherein the plurality of pathogens or infectious agents are HIV-1, HIV-2, HCV, HBV, and Malaria.

R40. The method of R8, wherein the plurality of pathogens or infectious agents are HIV-1, HIV-2, HCV, HBV, Malaria, and Babesia.

R41. The method of R8, wherein the plurality of pathogens or infectious agents are HIV-1, HIV-2, HCV, HBV, and Dengue Virus.

R42. The method of R8, wherein the plurality of pathogens or infectious agents are HIV-1, HIV-2, HCV, HBV, Dengue Virus, and Chikungunya Virus.

R43. The method of R8, wherein the plurality of pathogens or infectious agents are HIV-1, HIV-2, HCV, HBV, Dengue Virus, WNV, and Chikungunya Virus.

R44. The method of R8, wherein the plurality of pathogens or infectious agents are HIV-1, HIV-2, HCV, HBV, and Chikungunya Virus.

R45. The method of R8, wherein the plurality of pathogens or infectious agents are HIV-1, HIV-2, HCV, HBV, Chikungunya Virus, and Zika Virus.

R46. The method of R8, wherein the plurality of pathogens or infectious agents are HIV-1, HIV-2, HCV, HBV, Chikungunya Virus, Zika Virus, and WNV.

R47. The method of R8, wherein the plurality of pathogens or infectious agents are HIV-1, HIV-2, HCV, HBV, WNV, and Dengue Virus.

R48. The method of R8, wherein the plurality of pathogens or infectious agents are HIV-1, HIV-2, HCV, HBV, Chikungunya Virus, Zika Virus, and WNV.

R49. The method of R8, wherein: the plurality of pathogens or infectious agents are HIV-1, HIV-2, HCV, and HBV; and the nucleic acid analysis comprises multiplex analysis of HIV-1 and HIV-2.

R50. The method of R8, wherein: the plurality of pathogens or infectious agents are HIV-1, HIV-2, HCV, and HBV; and the nucleic acid analysis comprises multiplex analysis of HIV-1, HIV-2, and HCV.

R51. The method of R8, wherein: the plurality of pathogens or infectious agents are HIV-1, HIV-2, HCV, and HBV; and the nucleic acid analysis comprises multiplex analysis of HIV-1, HIV-2, and HBV.

R52. The method of R8, wherein: the plurality of pathogens or infectious agents are HIV-1, HIV-2, HCV, and HBV; and the nucleic acid analysis comprises multiplex analysis of HCV, and HBV.

R53. The method of R8, wherein: the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

R54. The method of R8, wherein: the plurality of pathogens or infectious agents are HIV-1, HIV-2, HCV, and HBV; and the nucleic acid analysis comprises multiplex analysis of HIV-1, HIV-2, HCV, and HBV.

R55. The method of R8, wherein: the plurality of pathogens or infectious agents comprise Zika Virus, WNV, Chikungunya Virus and Dengue Virus; the nucleic acid analysis comprises multiplex analysis of Zika Virus and WNV.

R56. The method of R8, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus.

R57. The method of R8, wherein: the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

R58. The method of R8, wherein: the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

R59. The method of R8, wherein: the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

R60. The method of R8, wherein: the plurality of pathogens or infectious agents comprise Babesia and Malaria; and the nucleic acid analysis comprises multiplex analysis of Babesia and Malaria.

R61. The method of R8, wherein: the plurality of pathogens or infectious agents comprise Parvovirus B19 and HAV; and the nucleic acid analysis comprises multiplex analysis of Parvovirus B19 and HAV.

R62. The method of R8, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; Parvovirus B19 at a predetermined level of at least 1-40 IU/mL; HAV at a predetermined level of at least 1-10 IU/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

R63. The method of R8, wherein the plurality of pathogens or infectious agents are HIV-1, HIV-2, HCV, and HBV; and wherein the predetermined levels are: at least 1-50 copies/mL of HIV-1; at least 1-20 IU/mL of HIV-2; at least 1-50 IU/mL of HCV; and at least 1-10 IU/mL of HBV.

R64. The method of R8, wherein the plurality of pathogens or infectious agents are HIV-1, HIV-2, HCV, HBV, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

R65. The method of R8, wherein the plurality of pathogens or infectious agents are Zika Virus and WNV; and wherein the predetermined levels are: at least 1-50 copies/mL of Zika Virus; and at least 1-50 copies/mL of WNV.

R66. The method of R8, wherein the plurality of pathogens or infectious agents are Chikungunya Virus and Dengue Virus; and wherein the predetermined levels are: at least 1-50 copies/mL of Chikungunya Virus; and at least 1-50 copies/mL of Dengue Virus.

R67. The method of R8, wherein the plurality of pathogens or infectious agents are Zika Virus, WNV, Chikungunya Virus and Dengue Virus; and wherein the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

R68. The method of R8, wherein the plurality of pathogens or infectious agents are Babesia and Malaria; and wherein the predetermined levels are: at least 1-20 copies/mL of Babesia; and at least 1-50 copies/mL of Malaria.

R69. The method of R8, wherein the plurality of pathogens or infectious agents are Parvovirus B19 and HAV; and wherein the predetermined levels are: at least 1-40 IU/mL of Parvovirus B19; and at least 1-10 IU/mL of HAV.

R70. The method of R8, wherein the plurality of pathogens or infectious agents are HIV-1, HIV-2, HCV, HBV, WNV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

R71. The method of R8, wherein the plurality of pathogens or infectious agents are HIV-1, HIV-2, HCV, HBV, WNV, Zika Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

R72. The method of R8, wherein the plurality of pathogens or infectious agents are HIV-1, HIV-2, HCV, HBV, WNV, Zika Virus, Chikungunya Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

R73. The method of R8, wherein the plurality of pathogens or infectious agents are HIV-1, HIV-2, HCV, HBV, WNV, Zika Virus, Chikungunya Virus, Dengue Virus, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

R74. The method of R8, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

R75. The method of R8, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

R76. The method of R8, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

R77. The method of R8, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

R78. The method of R8, wherein the plurality of pathogens or infectious agents are HIV-1, HIV-2, HCV, HBV, and Parvovirus B19; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

R79. The method of R8, wherein the plurality of pathogens or infectious agents are HIV-1, HIV-2, HCV, HBV, Parvovirus B19, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

R80. The method of R8, wherein the plurality of pathogens or infectious agents are HIV-1, HIV-2, HCV, HBV, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

R81. The method of R8, wherein the plurality of pathogens or infectious agents are HIV-1, HIV-2, HCV, HBV, and HAV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

R82. The method of R8, wherein the plurality of pathogens or infectious agents are HIV-1, HIV-2, HCV, HBV, and HEV; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

R83. The method of R8, wherein the plurality of pathogens or infectious agents are HIV-1, HIV-2, HCV, HBV, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

R84. The method of R8, wherein the plurality of pathogens or infectious agents are HIV-1, HIV-2, HCV, HBV, Zika Virus, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

R85. The method of R8, wherein the plurality of pathogens or infectious agents are HIV-1, HIV-2, HCV, HBV, Zika Virus, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

R86. The method of R8, wherein the plurality of pathogens or infectious agents are HIV-1, HIV-2, HCV, HBV, Zika Virus, Dengue Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

R87. The method of R8, wherein the plurality of pathogens or infectious agents are HIV-1, HIV-2, HCV, HBV, Zika Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

R88. The method of R8, wherein the plurality of pathogens or infectious agents are HIV-1, HIV-2, HCV, HBV, Zika Virus, WNV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

R89. The method of R8, wherein the plurality of pathogens or infectious agents are HIV-1, HIV-2, HCV, HBV, and Malaria; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

R90. The method of R8, wherein the plurality of pathogens or infectious agents are HIV-1, HIV-2, HCV, HBV, Malaria, and Babesia; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

R91. The method of R8, wherein the plurality of pathogens or infectious agents are HIV-1, HIV-2, HCV, HBV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

R92. The method of R8, wherein the plurality of pathogens or infectious agents are HIV-1, HIV-2, HCV, HBV, Dengue Virus, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

R93. The method of R8, wherein the plurality of pathogens or infectious agents are HIV-1, HIV-2, HCV, HBV, Dengue Virus, WNV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

R94. The method of R8, wherein the plurality of pathogens or infectious agents are HIV-1, HIV-2, HCV, HBV, and Chikungunya Virus; and wherein the predetermined levels are: at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

R95. The method of R8, wherein the plurality of pathogens or infectious agents are HIV-1, HIV-2, HCV, HBV, Chikungunya Virus, and Zika Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

R96. The method of R8, wherein the plurality of pathogens or infectious agents are HIV-1, HIV-2, HCV, HBV, Chikungunya Virus, Zika Virus, and WNV; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; at least 1-50 copies/mL of Zika Virus; and at least 1-50 copies/mL of WNV.

R97. The method of R8, wherein the plurality of pathogens or infectious agents are HIV-1, HIV-2, HCV, HBV, WNV, and Dengue Virus; and wherein the predetermined levels are: at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

R98. The method of R8, wherein: the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

R99. The method of R8, wherein: the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

R100. The method of R8, wherein: the plurality of pathogens or infectious agents are HIV-1, HIV-2, HCV, and HBV; the nucleic acid analysis comprises multiplex analysis of HIV-1, HIV-2, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

R101. The method of R8, wherein: the plurality of pathogens or infectious agents are HIV-1, HIV-2, HCV, and HBV; the nucleic acid analysis comprises multiplex analysis of HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

R102. The method of R8, wherein: the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are: at least 1-50 copies/mL of HIV-1; at least 1-20 IU/mL of HIV-2; at least 1-50 IU/mL of HCV; and at least 1-10 IU/mL of HBV.

R103. The method of R8, wherein: the plurality of pathogens or infectious agents are HIV-1, HIV-2, HCV, and HBV; the nucleic acid analysis comprises multiplex analysis of HIV-1, HIV-2, HCV, and HBV; and the predetermined levels are: at least 1-50 copies/mL of HIV-1; at least 1-20 IU/mL of HIV-2; at least 1-50 IU/mL of HCV; and at least 1-10 IU/mL of HBV.

R104. The method of R8, wherein: the plurality of pathogens or infectious agents comprise Zika Virus, WNV, Chikungunya Virus and Dengue Virus; the nucleic acid analysis comprises multiplex analysis of Zika Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

R105. The method of R8, wherein: the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

R106. The method of R8, wherein: the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

R107. The method of R8, wherein: the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

R108. The method of R8, wherein: the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

R109. The method of R8, wherein: the plurality of pathogens or infectious agents comprise Babesia and Malaria; the nucleic acid analysis comprises multiplex analysis of Babesia and Malaria; and the predetermined levels are: at least 1-50 copies/mL of Malaria; and at least 1-20 copies/mL of Babesia.

R110. The method of R8, wherein: the plurality of pathogens or infectious agents comprise Parvovirus B19 and HAV; the nucleic acid analysis comprises multiplex analysis of Parvovirus B19 and HAV; and the predetermined levels are: at least 1-40 IU/mL of Parvovirus B19; and at least 1-10 IU/mL of HAV.

R111. The method of any one of R-R110, wherein the nucleic acid analysis comprises a nucleic acid amplification reaction.

R112. The method of R111, wherein the nucleic acid amplification reaction is an isothermal reaction.

R113. The method of R112, wherein the isothermal reaction is recombinase polymerase amplification.

R113. The method of R112, wherein the isothermal reaction is a nicking enzyme amplification reaction.

R114. The method of any one of R-R113, wherein the nucleic acid analysis comprises optical detection of the presence of a nucleic acid derived from at least one of the plurality of pathogens or infectious agents.

R115. The method of any one of R-R114, wherein the nucleic acid analysis comprises digital detection of the presence of a nucleic acid derived from at least one of the plurality of pathogens or infectious agents.

R116. The method of any one of R-R115, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

R117. The method of R114, wherein the optical detection of the presence of a nucleic acid derived from at least one of the plurality of pathogens or infectious agents comprises optically detecting a fluorescent signal corresponding to the target nucleic acid at a plurality of times during the amplification reaction.

R118. The method of R116 or R117, wherein the plurality of times comprises optically detecting the signal at a predetermined interval of about every 20 seconds or about every 30 seconds.

R119. The method of R116, R117 or R118, wherein the optically detecting a fluorescent signal for a plurality of times occurs over a period of about 12 minutes.

R120. The method of R116, R117 or R118, wherein the optically detecting a fluorescent signal for a plurality of times occurs over a period of about 1 minute.

R121. The method of R116, R117 or R118, wherein the optically detecting a fluorescent signal for a plurality of times occurs over a period of about 5 minutes.

R122. The method of R116, R117 or R118, wherein the optically detecting a fluorescent signal for a plurality of times occurs over a period of about 20 minutes.

R124. The method of R116 or R117, wherein the plurality of times comprises optically detecting the signal at a predetermined interval of about every 48 seconds.

S. The present disclosure provides a method of performing a wash process for nucleic acid analysis, comprising: providing a wash vessel comprising a first well, a second well adjacent the first well, and a third well adjacent the second well, wherein a first side wall defines a first side of the first, second, and third wells, a second side wall opposite the first side wall, defines a second side of the first, second, and third wells, the first and second side walls comprise an inner surface facing the first, second, and third wells and an outer surface opposite the inner surface, a first divider separates the first and second well, the first side wall is higher than the first divider, the inner surface of the first side wall is substantially planar at least in a region extending between the first and second wells, a second divider separates the second and third wells, the inner surface of the second side wall is substantially planar at least in a region extending between the second and third wells, introducing magnetic microparticles having nucleic acid from a sample bound thereto into the first well, wherein the first well comprises a wash solution, applying a magnetic force to capture the magnetic microparticles on an inner surface of the first side wall in the first well; translating the captured magnetic microparticles from the first well over the first divider to the second well along the inner planar surface of the first side wall, wherein the second well comprises a wash solution; removing the magnetic force and allowing the magnetic microparticles to be released into the second well from the inner surface; applying a magnetic force to capture the magnetic microparticles on the inner surface of the second side wall opposite the first side wall, wherein the second side wall is higher than the second divider, translating the captured magnetic microparticles from the second well over the second divider to the third well along the inner planar surface of the second side wall, wherein the third well comprises a wash solution or an elution solution; and removing the magnetic force and allowing the magnetic microparticles to be released into the third well from the inner surface of the second side wall.

S1. The method of S, wherein the third well comprises an elution solution and the method further comprises, applying a magnetic force to the magnetic microparticles in the third well to capture the magnetic microparticles on an inner surface of the first side wall and removing the elution solution.

S2. The method of S, wherein the wash vessel comprises a fourth well and wherein the third well comprises a wash solution and the fourth well comprises an elution solution, and the third and fourth wells are separated by a third divider.

S3. The method of S2, wherein the first and second side walls extend along the first, second, third and fourth wells and define opposite sides of the wells, the inner surface of the first and second side wall faces the wells, the inner surface of the first side wall is non-planar in a region extending between the second and third wells, and planar in a region extending between the third and the fourth wells, the inner surface of the second side wall is non-planar in a region extending between the third and fourth wells, and the planar regions are conducive to translation of captured magnetic microparticles across the inner surface and the non-planar regions do not allow translation of captured magnetic microparticles across the inner surface.

S4. The method of S3, further comprising: applying a magnetic force to capture the magnetic particles in the third well on an inner surface of the first side wall, wherein the first side wall is higher than the third divider; translating the captured magnetic microparticles from the third well over the third divider to the fourth well along the inner surface of the first side wall; and removing the magnetic force and allowing the magnetic microparticles to be released into the fourth well from the inner surface of the first side wall.

S5. The method of S4, wherein the third well comprises a wash solution and the fourth well comprises an elution solution and the method further comprises applying a magnetic force to the magnetic microparticles in the fourth well to capture the magnetic microparticles on an inner surface of the second side wall and removing the elution solution.

S6. The method of S-S5, wherein the magnetic force is produced using an electromagnet.

S7. The method of S6, wherein the electromagnet is located along the outer surface of the first and second side walls and the translating the captured magnetic microparticles comprises sequentially activating and deactivating different regions of the electromagnet.

S8. The method of S-S5, wherein the magnetic force is produced using a permanent magnet.

S9. The method of S8, wherein the translating the captured magnetic microparticles comprises moving the permanent magnet along the outer surface of the first or the second side wall.

S10. The method of S-S8, wherein the non-planar inner surface comprises a notch at the inner surface, wherein the notch extends out of the inner surface or extends into the inner surface.

S11. The method of any one of S-S10, wherein the nucleic acid analysis comprises a nucleic acid amplification reaction.

S12. The method of S11, wherein the nucleic acid amplification reaction is an isothermal reaction.

S13. The method of S12, wherein the isothermal reaction is recombinase polymerase amplification.

S14. The method of S12, wherein the isothermal reaction is a nicking enzyme amplification reaction.

S15. The method of any one of S-S14, wherein the nucleic acid analysis comprises optical detection of the presence of a nucleic acid derived from at least one of the plurality of pathogens or infectious agents.

S16. The method of any one of S-S15, wherein the nucleic acid analysis comprises digital detection of the presence of a nucleic acid derived from at least one of the plurality of pathogens or infectious agents.

S17. The method of S15, wherein the optical detection of the presence of a nucleic acid derived from at least one of the plurality of pathogens or infectious agents comprises optically detecting a fluorescent signal corresponding to the target nucleic acid at a plurality of times during the amplification reaction.

S18. The method of any one of S-S16, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

S19. The method of S17 or S18, wherein the plurality of times comprises optically detecting the signal at a predetermined interval of about every 20 seconds or about every 30 seconds.

S19. The method of any one of S17-S19, wherein the optically detecting a fluorescent signal for a plurality of times occurs over a period of about 12 minutes.

S20. The method of any one of S17-S19, wherein the optically detecting a fluorescent signal for a plurality of times occurs over a period of about 1 minute.

S21. The method of any one of S17-S19, wherein the optically detecting a fluorescent signal for a plurality of times occurs over a period of about 5 minutes.

S22. The method of any one of S17-S19, wherein the optically detecting a fluorescent signal for a plurality of times occurs over a period of about 20 minutes.

S24. The method of any one of S17-S19, wherein the plurality of times comprises optically detecting the signal at a predetermined interval of about every 48 seconds.

T. The present disclosure provides a method for performing a nucleic acid analysis on a sample to detect one or more pathogens or infectious agents, comprising: performing a sample preparation process, an amplification process, and a detection process to detect one or more pathogens or infectious agents in the sample, wherein the sample preparation process includes: transporting the sample and one or more reagents in a vessel on a sample transport along a transport path from a sample dispense position to a sample capture and transfer position, and successively oscillating the sample transport to mix the sample and one or more reagents in the vessel, the vessel moving in an arc with each oscillation.

T1. The method of T, wherein the sample transport includes a serpentine path, carousel, conveyor, extrusion, robotic handler, or belt.

T2. The method of any one of T-T1, wherein successively oscillating the sample transport includes successively moving the sample transport between about 1 and about 5 degrees clockwise followed by moving the sample transport between about 1 and about 5 degrees counterclockwise.

T3. The method of any one of T-T2, wherein mixing the sample and one or more reagents in the vessel includes forming a vortex within the contents of the vessel.

T4. The method of T3, wherein the vortex is formed in about 1 second to about 2 seconds.

T5. The method of any one of T-T4, wherein the vessel has a draft angle of between about 0.5 degrees and about 1.5 degrees.

T6. The method of any one of T-T5, wherein the vessel has a conical bottom.

T7. The method of any one of T-T6, wherein the method further comprises aspirating the sample from the vessel while the sample transport is successively oscillating.

T8. The method of any one of T-T7, wherein the nucleic acid analysis comprises a nucleic acid amplification reaction.

T9. The method of T8, wherein the nucleic acid amplification reaction is an isothermal reaction.

T10. The method of T9, wherein the isothermal reaction is recombinase polymerase amplification.

T11. The method of T9, wherein the isothermal reaction is a nicking enzyme amplification reaction.

T12. The method of any one of T-T11, wherein the nucleic acid analysis comprises optical detection of the presence of a nucleic acid derived from at least one of the plurality of pathogens or infectious agents.

T13. The method of any one of T-T12, wherein the nucleic acid analysis comprises digital detection of the presence of a nucleic acid derived from at least one of the plurality of pathogens or infectious agents.

T14. The method of any one of T-T13, wherein the nucleic acid analysis includes a determination of a predetermined level of nucleic acids, the determination comprising optically detecting a fluorescent signal corresponding to the predetermined level of a target nucleic acid at a plurality of times during the amplification reaction.

T14-1. The method of any one of T-T13, wherein the optical detection of the presence of a nucleic acid derived from at least one of the plurality of pathogens or infectious agents comprises optically detecting a fluorescent signal corresponding to the target nucleic acid at a plurality of times during the amplification reaction.

T15. The method of T14 or T14-1, wherein the plurality of times comprises optically detecting the signal at a predetermined interval of about every 20 seconds or about every 30 seconds.

T16. The method of T14, T14-1 or T15, wherein the optically detecting a fluorescent signal for a plurality of times occurs over a period of about 12 minutes.

T17. The method of any one of T14, T14-1 or T15, wherein the optically detecting a fluorescent signal for a plurality of times occurs over a period of about 1 minute.

T18. The method of any one of T14, T14-1 or T15, wherein the optically detecting a fluorescent signal for a plurality of times occurs over a period of about 5 minutes.

T19. The method of any one of T14, T14-1 or T15, wherein the optically detecting a fluorescent signal for a plurality of times occurs over a period of about 20 minutes.

T20. The method of any one of T14, T14-1 or T15, wherein the plurality of times comprises optically detecting the signal at a predetermined interval of about every 48 seconds.

T21. The method of any one of A-T20, wherein the nucleic acid analysis is completed in about 15 to about 36 minutes.

T22. The method of any one of A-T21, wherein the nucleic acid analysis is completed in about 32 to about 36 minutes.

T23. The method of A-T22, wherein the nucleic acid analysis comprises a sample preparation process and an amplification and detection process.

T24. The method of any one of A-T23, wherein the sample preparation process comprises a lysis process and a wash process.

T25. The method of any one of A-T24, wherein the sample preparation process is completed in about 12 to about 16 minutes.

T26. The method of any one of A-T25, wherein the amplification and detection process is completed in about 1 to about 22 minutes.

T27. The method of any one of A-T26, wherein the amplification and detection process is completed in about 5 to about 22 minutes.

T28. The method of any one of A-T27, wherein the amplification and detection process is completed in about 18 to about 22 minutes.

T29. The method of any one of A-T28, wherein the lysis process is completed in about 4 to about 8 minutes.

T30. The method of any one of A-T29, wherein the wash process is completed in about 5 to about 9 minutes.

T31. The method of any one of A-T30, wherein the wash process comprises a first wash step, a second wash step, a third wash step and an elution step.

T32. The method of any one of A-T31, wherein the first wash step is completed in about 1 to about 3 minutes.

T33. The method of any one of A-T32, wherein the second wash step is completed in about 0.1 to about 1 minute.

T34. The method of any one of A-T33, wherein the third wash step is completed in about 0.1 to about 1 minute.

T35. The method of any one of A-T34, wherein the elution step is completed in about 2 to about 5 minutes.

T36. The method of any one of A-T35, wherein the sample preparation process further comprises a pre-treatment process.

T37. The method of any one of A-T36, wherein the pre-treatment process is completed in about 3 to about 7 minutes.

T38. The method of any one of A-T37, wherein the sample preparation process further comprises an onboard pooling process, and wherein up to 6 samples of donor blood are pre-treated and pooled in about 6 minutes or less.

T39. The method of any one of A-T38, wherein the sample preparation process further comprises an onboard pooling process, and wherein up to 24 samples are pooled in about 5 minutes or less.

T40. The method of any one of T31-T39, wherein the first wash step comprises incubation in a lysis buffer.

T41. The method of any one of T31-T40, wherein the second wash step comprises incubation in water.

T42. The method of any one of T31-T41, wherein the third wash step comprises incubation in water.

T43. The method of any one of T31-T42, wherein the elution step comprises incubation in an elution buffer.

T44. The method of any one of T31-T40, wherein the first wash step comprises incubation in a lysis buffer, the second wash step comprises incubation in water, the third wash step comprises incubation in water and the elution step comprises incubation in an elution buffer.

T45. The method of any one of T24-T44, wherein the lysis step comprises contacting the sample with a composition comprising lysis buffer and microparticles.

T46. The method of any one of T24-T44, wherein the composition further comprises internal control nucleic acids.

T47. The method of any one of T23-T46, wherein the amplification and detection process comprises an amplification reaction.

T48. The method of T47, wherein the amplification reaction is an isothermal reaction.

T49. The method of T47, wherein the isothermal reaction is RPA.

T50. The method of any one of T47-T49, wherein the amplification reaction comprises contacting the eluate generated by the wash process with a composition comprising a recombinase (e.g., UvsX), a recombinase loading agent (e.g., UvsY) and a single strand DNA binding protein (e.g., Gp32).

T51. The method of any one of T47-T50, wherein the amplification reaction comprises contacting the eluate generated by the wash process with a composition comprising about 5 mM to about 100 mM Tris-HCl at pH of about 6.5-9.0, e.g., 8.3; about 5 mM to about 10 mM DTT; about 50 mM to about 100 mM Potassium Acetate; about 2% to about 10% of a crowding agent, e.g., PEG; about 1 mM to about 5 mM dNTPs; about 1 mM to about 10 mM of ATP, e.g., about 2 mM to about 5 mM ATP; about 20 mM to about 100 mM Phosphocreatine, e.g., about 40 mM to about 100 mM Phosphocreatine; about 5 mM to about 40 mM Mg Acetate, e.g., about 10 mM to about 40 mM Mg Acetate; about 0.01 mg/mL to about 10 mg/mL BSA; about 5% to about 10% Glycerol; about 0.01 mg/mL to about 0.5 mg/mL Creatine Kinase, e.g., about 0.1 mg/mL to about 0.5 mg/mL Creatine Kinase; about 0.1 mg/mL to about 1.0 mg/mL UvsX, e.g., about 0.3 mg/mL to about 1.0 mg/mL UvsX; about 0.01 mg/mL to about 0.25 mg/mL UvsY, e.g., about 0.09 mg/mL to about 0.25 mg/mL UvsY; about 0.01 mg/mL to about 0.5 mg/mL DNA Polymerase, e.g., about 0.08 mg/mL to about 0.2 mg/mL DNA Polymerase; about 0.1 mg/mL to about 2.0 mg/mL GP32, e.g., about 0.4 mg/mL to about 0.8 mg/mL GP32; about 0.01 mg/mL to about 0.5 mg/mL Exonuclease III, e.g., about 0.1 mg/mL to about 0.5 mg/mL Exonuclease III; and about 0.5 μg/mL to about 20.0 μg/mL equine infectious anemia virus reverse transcriptase (EIAV-RT), e.g., about 0.5 μg/mL to about 1.5 μg/mL equine infectious anemia virus reverse transcriptase EIAV-RT.

T51-1. The method of any one of T24-T51, wherein the lysis process comprises contacting the sample with a lysis buffer.

T51-2. The method of T51-1, wherein the lysis buffer comprises a protease, a detergent, a protein denaturant and a buffer.

T51-3. The method of T51-1, wherein the lysis buffer comprises a protease, a detergent, a chaotropic agent and a buffer.

T51-4. The method of T51-3, wherein the chaotropic agent is guanidinium thiocyanate (GITC).

T51-5. The method of any one of T51-1-T51-4, wherein the lysis buffer has pH from about 6.0 to about 8.0.

T51-6. The method of any one of T51-1-T51-5, wherein the lysis buffer has pH from about 7.0 to about 8.0.

T52. The method of any one of A-T51-5, further comprising treating donor material indicated for release for clinical use or donor blood determined to be acceptable for transfusion with a pathogen reduction technology.

T53. The method of T52, wherein the pathogen reduction technology comprises an agent that intercalates into nucleic acids present within the donor material or donor blood or causes dimers in nucleic acids present within the donor material or donor blood.

T54. The method of T52, wherein the pathogen reduction technology comprises at least one of the following: (i) amotosalen and Ultraviolet A (UVA) light, (ii) riboflavin and UVA/Ultraviolet B (UVB) light, (iii) amustaline and glutathione, (iv) Ultraviolet C (UVC) light; and (v) methylene blue and visible light T55. The method of any one of T52-T54, wherein the donor material comprises at least one of the following: (i) whole blood, (ii) plasma, (iii) platelets, (iv) red blood cells; and (v) plasma-derived products.

T56. The method of any one of T52-T55, wherein the method further comprises: performing an immunoassay on a second portion of the sample or sample of donor blood for detecting one or more pathogens or infectious agents in the sample or sample of donor blood.

T57. The method of T56, wherein the immunoassay is performed simultaneously with the nucleic acid analysis.

T58. The method of T56, wherein the immunoassay is performed before the nucleic acid analysis.

T59. The method of T56, wherein the nucleic acid analysis is performed before the immunoassay is performed.

T60. The method of T56, wherein the immunoassay is performed before the donor material indicated for release for clinical use or donor blood determined to be acceptable for transfusion is treated with the pathogen reduction technology.

T61. The method of any one of T-T61, wherein the pathogen or infectious agent is selected from the group consisting of: SARS-CoV-2 (COVID-19), coronaviruses, HIV-1, HIV-2, HBV, HCV, CMV, Epstein-Barr virus (EBV), human T-lymphotropic virus (HTLV), Parvovirus B19, HAV, syphilis, Chlamydia, Gonorrhea, Dengue, Chikungunya, WNV, HEV, Creutzfeldt-Jakob disease (vCJD) and a combination thereof.

T62. The method of any one of T52-T61, wherein the nucleic acid analysis and the pathogen reduction technology are performed in the same physical location.

T63. The method of T55, wherein the donor material comprises platelets.

T64. The method of T62, wherein the sample physical location is a decentralized location.

T65. The method of T62, wherein the sample physical location is a centralized location.

T66. The method of any of A-T65, wherein the nucleic acid analysis includes at least about 140 sample aspirations per hour.

T67. The method of any of A-T66, wherein the nucleic acid analysis includes an amplification process including at least about 140 amplifications per hour.

T68. The method of any of A-T67, wherein the nucleic acid analysis includes an amplification process including multiplex amplification.

T69. The method of any of A-T68, wherein the nucleic acid analysis includes a sample preparation process including preparing eluate and wherein the eluate is split into two or more amplification vessels.

T70. The method of any of A-T69, wherein the nucleic acid analysis is performed on an automated system and wherein between about 140 results and about 300 results are obtained per hour per m² of a footprint of the automated system.

T71. The method of any one of A-T70, further comprising performing a serological test on the sample of donor blood, the donor material, the plasma or the biological sample.

T72. The method of any one of A-T71, wherein the sample of donor blood, sample, or biological sample is a pooled sample.

T73. The method of T72, wherein the pooled sample comprises blood or plasma or serum or biological samples from 2 donors.

T74. The method of T72, wherein the pooled sample comprises blood or plasma or serum or biological samples from 3 donors.

T75. The method of T72, wherein the pooled sample comprises blood or plasma or serum or biological samples from 4 donors.

T76. The method of T72, wherein the pooled sample comprises blood or plasma or serum or biological samples from 5 donors.

T77. The method of T72, wherein the pooled sample comprises blood or plasma or serum or biological samples from 10 donors.

T78. The method of T72, wherein the pooled sample comprises blood or plasma or serum or biological samples from 12 donors.

T79. The method of T72, wherein the pooled sample comprises blood or plasma or serum or biological samples from 96 donors.

T80. The method of any of A-T79, wherein the nucleic acid analysis includes a sample preparation process comprising onboard pooling of a subset of a plurality of samples to form a pooled sample, the nucleic acid analysis being performed on the pooled sample.

T82. The method of claim T80, wherein onboard pooling includes continually transporting individual vessels on a sample transport along a transport path from a sample dispense position to a sample capture and transfer position, with intermediate positions therebetween; and pooling a first sample and a second sample in a vessel on the sample transport to form a pooled sample.

T83. The method of T82, wherein the sample transport includes a serpentine path, carousel, conveyor, extrusion, robotic handler, or belt.

T84. The method of T82, wherein the first sample and the second sample are individual donor samples.

T85. The method of T82, wherein the first sample and the second sample are pooled samples.

T86. The method of T82, wherein the first sample and the second sample each include 48 pooled samples.

T87. The method of T82, wherein the sample transport includes a sample preparation carousel and wherein continually transporting individual vessels along the transport path includes rotating the sample preparation carousel.

T88. The method of T87, wherein the sample preparation carousel rotates in a lockstep fashion, moving one position with each lockstep.

T89. The method of T88, wherein the duration of each lockstep is between about 15 and about 30 seconds.

T90. The method of any of T82-T89, wherein pooling the first sample and the second sample includes dispensing the first sample and the second sample into a first vessel at the sample dispense position prior to the sample preparation carousel transporting the first vessel to a first intermediate position.

T91. The method of T90, further comprising dispensing third and fourth samples into the first vessel at the first intermediate position to add the third and fourth samples to the pooled sample.

T92. The method of any of T90-T91, wherein the sample preparation process comprises a lysis process, and wherein onboard pooling includes aspirating the pooled sample from the first vessel and dispensing the pooled sample into a second vessel at the sample dispense position to initiate the lysis process for the pooled sample.

T93. The methods of any of T80-T92, wherein the sample preparation process includes a pre-treatment process.

T94. The method of T93, wherein the pre-treatment process includes dispensing a sample and reagent into a vessel on the sample transport at the sample dispense position, continually transporting the vessel along the transport path defined between the sample dispense position and the capture and transfer position, with intermediate positions therebetween; and mixing the sample and reagent at each intermediate position.

T95. The method of T94, wherein mixing the sample and reagent includes successively oscillating the sample transport to mix the sample and reagent in the vessel, the vessel moving in an arc with each oscillation.

T96. The method of any of T93-T95, wherein the sample includes a whole blood sample and wherein the reagent includes a lysis buffer.

T97. The method of T96, wherein the pre-treatment process includes lysing the sample for between about 3 and about 6 minutes.

T98. The method of any of T93-T97, further comprising onboard pooling of a subset of pre-treated samples to form a pooled sample, the nucleic acid analysis being performed on the pooled sample, and wherein onboard pooling includes aspirating a first pre-treated sample from a first vessel at a first intermediate position, aspirating a second pre-treated sample from a second vessel at a second intermediate position, and dispensing the first pre-treated sample and second pre-treated sample in a third vessel at the sample dispense position.

T99. The method of any of T80-T99, wherein up to about 300 samples are pooled for nucleic acid analysis per hour.

T100. The method of T98, wherein up to about 75 pre-treated samples are pooled for nucleic acid analysis per hour.

T101. The method of T100, wherein the nucleic acid analysis includes detection of one or more of a plurality of pathogens or infectious agents.

T102. The method of T101, wherein a determination of a presence or absence of nucleic acids derived from each of the plurality of pathogens or infectious agents based on the nucleic acid analysis of the pooled sample is indicative of whether (a) donor blood associated with the pooled sample is acceptable for transfusion or (b) a donor material associated with the pooled sample is acceptable for clinical use.

T103. The method of T102, wherein upon a determination of the presence of a nucleic acid derived from at least one of the plurality of pathogens or infectious agents in a pooled sample, the method further comprises nucleic acid analysis of individual samples or sub-pools thereof included in the pooled sample and making a determination whether (a) donor blood associated with individual samples or sub-pools thereof is acceptable for transfusion based at least in part on the nucleic acid analysis of the individual samples or sub-pools thereof or (b) a donor material associated with individual samples or sub-pools thereof is acceptable for clinical use based at least in part on the nucleic acid analysis of the individual samples or sub-pools thereof.

T104. The method of T103, wherein individual samples included in the pooled sample are stored in a loading area during nucleic acid analysis of the pooled sample.

T105. The method of any of T103-T104, wherein nucleic acid analysis of sub-pools includes onboard pooling to create the sub-pools.

T106. The method of any of T103-T105, wherein the pooled sample includes 96 individual donor samples, and wherein upon a determination of the presence of a nucleic acid derived from at least one of the plurality of pathogens or infectious agents in the pooled sample, a determination whether donor blood associated with each of the 96 individual samples is acceptable for transfusion is made in less than about 4 hours from initial aspiration of a first sample included in the pooled sample for performance of the nucleic acid analysis.

T107. The method of any one of A-T106, wherein the nucleic acid analysis comprises a sample preparation process.

T108. The method of T107, wherein the sample preparation process comprises a lysis process.

T109. The method of T107, wherein the sample preparation process comprises a pre-treatment process and a lysis process.

T110. The method of T107, wherein the sample preparation process comprises a pre-treatment process and an onboard pooling process.

T111. The method of T107, wherein the sample preparation process comprises a pre-treatment process, a lysis process and an onboard pooling process.

T112. The method of T107, wherein the sample preparation process comprises an onboard pooling process and a lysis process.

T113. The method of T108, wherein the sample preparation process comprises an onboard pooling process.

T113. The method of T109, T110 or T111, wherein the pre-treatment process comprises pre-treatment lysis process.

T114. The method of any one of T23, wherein the sample preparation process of the present disclosure can use microparticles.

T115. The method of T114, wherein the microparticles comprises CuTi-coated microparticles, e.g., to capture total sample nucleic acid.

T116. The method of T114 and T115, wherein the microparticles are present in a lysis buffer.

T117, The method of T115 or T116, wherein the microparticles are present in a lysis buffer comprising internal control nucleic acids and, optionally, a proteinase.

T118. The method of T116, wherein the lysis buffer comprises a detergent, a protein denaturant and a buffer and, optionally, a protease.

T119. The method of T116, wherein the lysis buffer comprises a detergent, a chaotropic agent and a buffer and, optionally, a protease.

T120. The method of T116, wherein the lysis buffer comprises 2.5 M to about 4.7 M GITC and about 2% to about 10% Tween-20, and a pH of about 5.5 to about 8.0.

T121. The method of any one of T31-T120, wherein the first wash step comprises an incubation in a first wash solution, e.g., comprising a lysis buffer.

T122. The method of any one of T31-T121, wherein the second wash step comprises an incubation in water.

T123. The method of any one of T31-T122, wherein the third wash step comprises an incubation in water.

T124. The method of any one of T32-T123, wherein the elution step comprises an incubation in an elution buffer comprising phosphate, e.g., at a concentration of about 5 mM to about 10 mM.

T125. The method of T124, wherein the elution buffer comprises PO₄.

T126. The method of T124 or T125, wherein the elution buffer has a pH from about 7.5 to about 9.0.

T127. The method of any one of T47-T126, wherein the amplification reaction comprises contacting the isolated nucleic acids with a solution comprising gp32, UvsX, UvsY, and a Polymerase.

T128. The method of T127, wherein the solution further comprises ATP, Phosphocreatine and a buffer.

T129. The method of T127 or T128, wherein the solution further comprises further comprises a reverse transcriptase.

T130. The method of any one of T47-T126, wherein the amplification reaction comprises contacting the isolated nucleic acids with a solution comprising (i) a recombinase (e.g., UvsX), (ii) a single strand binding protein (e.g., GP32), (iii) a recombinase loading agent (e.g., UvsY), (iv) a DNA polymerase, (v) an exonuclease (e.g., Exonuclease III), and (vi) Creatine Kinase.

T131. The method of T130, wherein the solution further comprises a reverse transcriptase (e.g., EIAV-RT).

T132. The method of any one of T127-T131, wherein the solution further comprises a forward primer, a reverse primer and a probe.

T133. The method of any one of T23-T132, wherein the amplification and detection process comprises optically detecting a fluorescent signal corresponding to the target nucleic acid at a plurality of times during the amplification reaction.

T134. The method of T133, wherein the plurality of times during the amplification process comprises the detecting a fluorescent signal every about 20 to about 30 seconds, e.g., about 24 seconds.

U. The present disclosure provides an automated system for screening a sample of donor blood for release of a donor material for clinical use, comprising: a sample analysis station comprising, a sample loading area, a sample preparation area, a nucleic acid amplification area and a nucleic acid detection area; a processor and memory comprising instructions, when executed by the processor cause the system to: perform a nucleic acid analysis on the sample of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor material for clinical use; and wherein the release of the donor material for clinical use occurs in about 15 to about 60 minutes from initial aspiration of the sample of donor blood for performance of the nucleic acid analysis.

U1. The system of U, wherein release of the donor material for clinical use occurs in about 20 to about 60 minutes from initial aspiration of the sample of donor blood for performance of the nucleic acid analysis.

U2. The system of any one of U-U1, wherein the nucleic acid analysis comprises a nucleic acid amplification reaction of about 8 minutes to about 20 minutes in duration.

U3. The system of any one of U-U2, wherein each determination of a predetermined level of nucleic acid from at least one of the plurality of pathogens or infectious agent is completed in about 30 to about 45 minutes from initial aspiration of the sample of donor blood for performance of the nucleic acid analysis.

U4. The system of any one of U-U3, wherein each determination of a predetermined level of nucleic acid from at least one of the plurality of pathogens or infectious agent has a time to result of about 30 to about 45 minutes.

U5. The system of any one of U-U4, wherein at least about 70 results are obtained per hour per m³ of a volume occupied by the automated system.

U6. The system of any one of U-U5, wherein at least about 140 results are obtained per hour per m² of a footprint of the automated system.

V. The present disclosure provides an automated system for screening a sample of donor blood for release of a donor material for clinical use, comprising: a sample analysis station comprising, a sample loading area, a sample preparation area, a nucleic acid amplification area and a nucleic acid detection area; a processor and memory comprising instructions, when executed by the processor cause the system to: perform a nucleic acid analysis on the sample of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor material for clinical use; and wherein the system is configured to screen a plurality of samples of donor blood for release of the donor material for clinical use and the determinations based on the nucleic acid analyses are performed within about 20 minutes to about 3.5 hours from initial aspiration of the first sample of donor blood for performance of the nucleic acid analysis.

W. The present disclosure provides an automated system for screening a sample of donor blood for release of a donor material for clinical use, comprising a sample analysis station comprising, a sample loading area, a sample preparation area, a nucleic acid amplification area and a nucleic acid detection area; a processor and memory comprising instructions, when executed by the processor cause the system to: perform a nucleic acid analysis on the sample of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor material for clinical use; and wherein the nucleic acid analysis comprises a nucleic acid amplification reaction of about 8 minutes to about 20 minutes in duration.

X. The present disclosure provides an automated system for screening a sample of donor blood for release of a donor material for clinical use, comprising: a sample analysis station comprising, a sample loading area, a sample preparation area, a nucleic acid amplification area and a nucleic acid detection area; a processor and memory comprising instructions, when executed by the processor cause the system to: perform a nucleic acid analysis on the sample of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor material for clinical use; and wherein each determination of a predetermined level of nucleic acid from at least one of the plurality of pathogens or infectious agent is completed in about 30 to about 45 minutes from initial aspiration of the sample of donor blood for performance of the nucleic acid analysis.

Y. The present disclosure provides an automated system for screening a sample of donor blood for release of a donor material for clinical use, comprising: a sample analysis station comprising, a sample loading area, a sample preparation area, a nucleic acid amplification area and a nucleic acid detection area; a processor and memory comprising instructions, when executed by the processor cause the system to: perform a nucleic acid analysis on the sample of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor material for clinical use; and wherein each determination of a predetermined level of nucleic acid from at least one of the plurality of pathogens or infectious agent has a time to result of about 30 to about 45 minutes.

Z. The present disclosure provides an automated system for screening samples of donor blood for release of a donor material for clinical use, comprising: a sample analysis station comprising, a sample loading area, a sample preparation area, a nucleic acid amplification area and a nucleic acid detection area; a processor and memory comprising instructions, when executed by the processor cause the system to: perform a nucleic acid analysis on each of the samples of donor blood to detect one or more of a plurality of pathogens or infectious agents; wherein upon a determination of a predetermined level of nucleic acids derived from each of the plurality of pathogens or infectious agents as a result of the nucleic acid analysis, the system is indicative of release of the donor material for clinical use; and wherein at least about 70 results are obtained per hour per m³ of a volume occupied by the automated system.

AA. The present disclosure provides an automated system for screening samples of donor blood for release of a donor material for clinical use, comprising: a sample analysis station comprising, a sample loading area, a sample preparation area, a nucleic acid amplification area and a nucleic acid detection area; a processor and memory comprising instructions, when executed by the processor cause the system to: perform a nucleic acid analysis on each of the sample of donor blood to detect one or more of a plurality of pathogens or infectious agents; wherein upon a determination of a predetermined level of nucleic acids derived from each of the plurality of pathogens or infectious agents as a result of the nucleic acid analysis, the system is indicative of release of the donor material for clinical use; and wherein at least about 140 results are obtained per hour per m² of a footprint of the automated system.

BB. The present disclosure provides an automated system for screening a sample of donor blood for release of a donor material for clinical use, comprising: a sample analysis station comprising, a sample loading area, a sample preparation area, a nucleic acid amplification area and a nucleic acid detection area; a processor and memory comprising instructions, when executed by the processor cause the system to: perform a nucleic acid analysis on the sample of donor blood to detect one or more of a plurality of pathogens or infectious agents; wherein upon a determination of a predetermined level of nucleic acids derived from each of the plurality of pathogens or infectious agents based on the nucleic acid analysis, the system is indicative of release of the donor material for clinical use; and wherein the system is configured to screen a plurality of samples of donor blood for release of the donor material for clinical use and the nucleic acid analysis is performed in the absence of batching of the plurality of the samples.

CC. The present disclosure provides an automated system for the detection of target nucleic acid derived from one or more pathogens or infectious agents in a sample, comprising: a sample analysis station comprising, a sample loading area, a sample preparation area, a nucleic acid amplification area and a nucleic acid detection area; a processor and memory comprising instructions, when executed by the processor cause the system to perform a nucleic acid analysis on the sample to detect target nucleic acid from one or more pathogens or infectious agents, the nucleic acid analysis comprising: a sample preparation process at the sample preparation area including preparing nucleic acid from the sample for an amplification reaction; an amplification process at the nucleic acid amplification area including amplifying the target nucleic acid with the amplification reaction; and a detection process at the nucleic acid detection area including determining an amount of target nucleic acid amplified in the reaction; wherein the sample preparation process, amplification process and detection process are completed in less than about 60 minutes from initial aspiration of the sample for the nucleic acid analysis and wherein at least about 140 results are obtained per hour per m² of a footprint of the automated system.

CC1. The system of CC, wherein the nucleic acid amplification reaction has a duration of about 8 minutes to about 20 minutes.

CC2. The system of any one of CC-CC1, wherein the sample preparation process, amplification process and detection process are completed in about 20 minutes to about 60 minutes.

CC3. The system of any one of CC-CC2, wherein the system is configured to detect target nucleic acid in a plurality of samples and wherein at least about 70 results are obtained per hour per m³ of a volume of the automated system.

CC4. The system of any one of CC-CC3, wherein the system is configured to detect target nucleic acid in a plurality of samples and wherein between about 140 results and about 300 results are obtained per hour per m² of the footprint of the system.

CC5. The system of any one of CC-CC4, wherein the target nucleic acid comprises nucleic acid from a plurality of pathogens or infectious agents.

CC6. The system of CC5, wherein upon a determination of a predetermined level of nucleic acids derived from each of the plurality of pathogens or infectious agents based on the nucleic acid analysis, the system is indicative of release of a donor material for clinical use.

CC7. The system of CC5, wherein upon a determination of the detection or absence of nucleic acids derived from each of the plurality of pathogens or infectious agents based on the nucleic acid analysis, the system is indicative of release of a donor material for clinical use.

CC8. The system of CC7, wherein upon the detection of nucleic acids derived from each of the plurality of pathogens or infectious agents based on the nucleic acid analysis the donor material is not released for clinical use.

CC9. The system of CC7, wherein upon the absence of the detection of nucleic acids derived from each of the plurality of pathogens or infectious agents based on the nucleic acid analysis the donor material is released for clinical use.

CC10. The system of any one of CC-CC9, wherein the sample transport includes a serpentine path, carousel, conveyor, extrusion, robotic handler, or belt.

CC11. The system of any one of CC-CC9, wherein the sample transport is configured to successively move between about 1 and about 5 degrees clockwise followed by between about 1 and about 5 degrees counterclockwise.

CC12. The system of any one of CC-CC9, wherein the sample transport is configured to form a vortex within the contents of the vessel.

CC13. The system of any one of CC-CC9, wherein the vessel has a draft angle of between about 0.5 degrees and about 1.5 degrees.

CC14. The system of any one of CC-CC9, wherein the vessel has a conical bottom.

CC15. The method of system of any one of CC-CC14, wherein the system further includes at least one pipettor, and wherein the at least one pipettor is configured to aspirate the one or more samples from the vessel while the sample transport is successively oscillating.

DD. The present disclosure provides a wash vessel for nucleic acid analysis comprising: a first well adjacent a second well and a third well adjacent the second well, a first side wall defining a first side of the first, second, and third wells, a second side wall opposite the first side wall and defining a second side of the first, second, and third wells, the first and second side walls comprising an inner surface facing the first, second, and third wells and an outer surface opposite the inner surface, a first divider separating the first and second well, a second divider separating the second and third wells, wherein: the first side wall is higher than the first divider, the inner surface of the first side wall is substantially planar at least in a region extending between the first and second wells for translation of magnetic microparticles captured in the first well over the first divider into the second well, the inner surface of the first side wall is non-planar in a region extending between the second and third wells such that the non-planar inner surface prevents translation of magnetic particles captured at a region of the inner surface in the second well to a region of the inner surface in the third well, the second side wall is higher than the second divider, the inner surface of the second side wall is substantially planar at least in a region extending between the second and third wells and provides a substantially planar surface for capture of magnetic microparticles in the second well and translation of the captured magnetic particles over the second divider to the third well.

DD1. The vessel of DD, wherein the first and second wells comprise a wash solution and the third well comprises a wash solution or an elution solution.

DD2. The vessel of DD, comprising: a fourth well adjacent the third well, a third divider separating the third and fourth wells, wherein: the first side wall defines a first side of the fourth well and the second side wall defines a second side the fourth well, the first side wall is higher than the third divider, the inner surface of the first side wall is substantially planar at least in a region extending between the third and fourth wells for translation of magnetic microparticles captured in the third well over the third divider into the fourth well, and the inner surface of the second side wall is non-planar in a region extending between the third and fourth wells such that the non-planar inner surface prevents translation of magnetic particles captured at a region of the inner surface of the second side wall in the third well to a region of the inner surface of the second side wall in the fourth well.

DD3. The vessel of DD2, wherein the first, second, and third wells comprise wash solution.

DD4. The vessel of any one of DD1-DD2, wherein the first, second, and third wells comprise wash solution and the fourth well comprises an elution solution.

DD5. The vessel of any one of DD-DD4, wherein the non-planar inner surface comprises a notch at the inner surface, wherein the notch extends out of the inner surface or extends into the inner surface.

DD6. The vessel of any one of DD, wherein the sample transport is configured to successively move between about 1 and about 5 degrees clockwise followed by between about 1 and about 5 degrees counterclockwise.

DD7. The vessel of any one of DD-DD1, wherein the sample transport is configured to form a vortex within the contents of the vessel.

DD8. The vessel of any one of DD7, where the sample transport is configured to form the vortex in about 1 second to about 2 seconds.

DD9. The vessel of any one of DD-DD3, wherein the vessel has a draft angle of between about 0.5 degrees and about 1.5 degrees.

DD10. The vessel of any one of DD-DD4, wherein the vessel has a conical bottom.

DD11. The vessel of any one of DD-DD5, wherein the system further includes at least one pipettor, and wherein the at least one pipettor is configured to aspirate the one or more samples from the vessel while the sample transport is successively oscillating.

EE. The present disclosure provides a system for performing nucleic acid analysis on one or more samples to detect one or more pathogens or infectious agents, comprising a sample analysis station comprising, a sample loading area, a sample preparation area, a nucleic acid amplification area and a nucleic acid detection area, wherein the sample preparation area includes: a sample transport configured to transport one or more samples in a vessel along a transport path from a sample dispense position to a sample capture and transfer position, the sample transport configured to successively oscillate to mix the one or more samples and one or more reagents in the vessel, the vessel moving in an arc with each oscillation.

EE1. The system of any one of U-U6, V, W, X, Y, Z, AA, BB, CC5 and CC6, wherein the plurality of pathogens or infectious agents are selected from the group consisting of: HIV-1, HIV-2, HBV, HCV, CMV, Parvovirus B19, HAV, Gonorrhea, WNV, Zika Virus, Dengue Virus, Chikungunya Virus, Babesia, Malaria, Usutu Virus and HEV.

EE2. The system of any one of U-U6, V, W, X, Y, Z, AA, BB, CC5 and CC6, wherein the plurality of pathogens or infectious agents are HIV-1, HIV-2, HCV, and HBV.

EE3. The system of any one of U-U6, V, W, X, Y, Z, AA, BB, CC5 and CC6, wherein the plurality of pathogens or infectious agents are HIV-1, HIV-2, HCV, HBV, and WNV.

EE4. The system of any one of U-U6, V, W, X, Y, Z, AA, BB, CC5 and CC6, wherein the plurality of pathogens or infectious agents are Zika Virus and WNV.

EE5. The system of any one of U-U6, V, W, X, Y, Z, AA, BB, CC5 and CC6, wherein the plurality of pathogens or infectious agents are Chikungunya Virus and Dengue Virus.

EE6. The system of any one of U-U6, V, W, X, Y, Z, AA, BB, CC5 and CC6, wherein the plurality of pathogens or infectious agents are Zika Virus, WNV, Chikungunya Virus and Dengue Virus.

EE7. The system of any one of U-U6, V, W, X, Y, Z, AA, BB, CC5 and CC6, wherein the plurality of pathogens or infectious agents are Babesia and Malaria.

EE8. The system of any one of U-U6, V, W, X, Y, Z, AA, BB, CC5 and CC6, wherein the plurality of pathogens or infectious agents are Parvovirus B19 and HAV.

EE9. The system of any one of U-U6, V, W, X, Y, Z, AA, BB, CC5 and CC6, wherein the plurality of pathogens or infectious agents are HIV-1, HIV-2, HCV, HBV, WNV, and Zika Virus.

EE10. The system of any one of U-U6, V, W, X, Y, Z, AA, BB, CC5 and CC6, wherein the plurality of pathogens or infectious agents are HIV-1, HIV-2, HCV, HBV, WNV, Zika Virus, and Chikungunya Virus.

EE11. The system of any one of U-U6, V, W, X, Y, Z, AA, BB, CC5 and CC6, wherein the plurality of pathogens or infectious agents are HIV-1, HIV-2, HCV, HBV, WNV, Zika Virus, Chikungunya Virus, and Dengue Virus.

EE12. The system of any one of U-U6, V, W, X, Y, Z, AA, BB, CC5 and CC6, wherein the plurality of pathogens or infectious agents are HIV-1, HIV-2, HCV, HBV, WNV, Zika Virus, Chikungunya Virus, Dengue Virus, and Babesia.

EE13. The system of any one of U-U6, V, W, X, Y, Z, AA, BB, CC5 and CC6, wherein the plurality of pathogens or infectious agents are HIV-1, HIV-2, HCV, HBV, WNV, Zika Virus, Chikungunya Virus, Dengue Virus, Babesia, and Malaria.

EE14. The system of any one of U-U6, V, W, X, Y, Z, AA, BB, CC5 and CC6, wherein the plurality of pathogens or infectious agents are HIV-1, HIV-2, HCV, HBV, WNV, Zika Virus, Chikungunya Virus, Dengue Virus, Babesia, Malaria, and Parvovirus B19.

EE 15. The system of any one of U-U6, V, W, X, Y, Z, AA, BB, CC5 and CC6, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV.

EE16. The system of any one of U-U6, V, W, X, Y, Z, AA, BB, CC5 and CC6, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV.

EE17. The system of any one of U-U6, V, W, X, Y, Z, AA, BB, CC5 and CC6, wherein the plurality of pathogens or infectious agents are HIV-1, HIV-2, HCV, HBV, and Parvovirus B19.

EE18. The system of any one of U-U6, V, W, X, Y, Z, AA, BB, CC5 and CC6, wherein the plurality of pathogens or infectious agents are HIV-1, HIV-2, HCV, HBV, Parvovirus B19, and HAV.

EE19. The system of any one of U-U6, V, W, X, Y, Z, AA, BB, CC5 and CC6, wherein the plurality of pathogens or infectious agents are HIV-1, HIV-2, HCV, HBV, and Babesia.

EE20. The system of any one of U-U6, V, W, X, Y, Z, AA, BB, CC5 and CC6, wherein the plurality of pathogens or infectious agents are HIV-1, HIV-2, HCV, HBV, and HAV.

EE21. The system of any one of U-U6, V, W, X, Y, Z, AA, BB, CC5 and CC6, wherein the plurality of pathogens or infectious agents are HIV-1, HIV-2, HCV, HBV, and HEV.

EE22. The system of any one of U-U6, V, W, X, Y, Z, AA, BB, CC5 and CC6, wherein the plurality of pathogens or infectious agents are HIV-1, HIV-2, HCV, HBV, and Zika Virus.

EE23. The system of any one of U-U6, V, W, X, Y, Z, AA, BB, CC5 and CC6, wherein the plurality of pathogens or infectious agents are HIV-1, HIV-2, HCV, HBV, Zika Virus, and Dengue Virus.

EE24. The system of any one of U-U6, V, W, X, Y, Z, AA, BB, CC5 and CC6, wherein the plurality of pathogens or infectious agents are HIV-1, HIV-2, HCV, HBV, Zika Virus, Dengue Virus, and Chikungunya Virus.

EE25. The system of any one of U-U6, V, W, X, Y, Z, AA, BB, CC5 and CC6, wherein the plurality of pathogens or infectious agents are HIV-1, HIV-2, HCV, HBV, Zika Virus, Dengue Virus, and WNV.

EE26. The system of any one of U-U6, V, W, X, Y, Z, AA, BB, CC5 and CC6, wherein the plurality of pathogens or infectious agents are HIV-1, HIV-2, HCV, HBV, Zika Virus, WNV, and Chikungunya Virus.

EE27. The system of any one of U-U6, V, W, X, Y, Z, AA, BB, CC5 and CC6, wherein the plurality of pathogens or infectious agents are HIV-1, HIV-2, HCV, HBV, Zika Virus, WNV, Dengue Virus, and Chikungunya Virus.

EE28. The system of any one of U-U6, V, W, X, Y, Z, AA, BB, CC5 and CC6, wherein the plurality of pathogens or infectious agents are HIV-1, HIV-2, HCV, HBV, and Malaria.

EE29. The system of any one of U-U6, V, W, X, Y, Z, AA, BB, CC5 and CC6, wherein the plurality of pathogens or infectious agents are HIV-1, HIV-2, HCV, HBV, Malaria, and Babesia.

EE30. The system of any one of U-U6, V, W, X, Y, Z, AA, BB, CC5 and CC6, wherein the plurality of pathogens or infectious agents are HIV-1, HIV-2, HCV, HBV, and Dengue Virus.

EE31. The system of any one of U-U6, V, W, X, Y, Z, AA, BB, CC5 and CC6, wherein the plurality of pathogens or infectious agents are HIV-1, HIV-2, HCV, HBV, Dengue Virus, and Chikungunya Virus.

EE32. The system of any one of U-U6, V, W, X, Y, Z, AA, BB, CC5 and CC6, wherein the plurality of pathogens or infectious agents are HIV-1, HIV-2, HCV, HBV, Dengue Virus, WNV, and Chikungunya Virus.

EE33. The system of any one of U-U6, V, W, X, Y, Z, AA, BB, CC5 and CC6, wherein the plurality of pathogens or infectious agents are HIV-1, HIV-2, HCV, HBV, and Chikungunya Virus.

EE34. The system of any one of U-U6, V, W, X, Y, Z, AA, BB, CC5 and CC6, wherein the plurality of pathogens or infectious agents are HIV-1, HIV-2, HCV, HBV, Chikungunya Virus, and Zika Virus.

EE35. The system of any one of U-U6, V, W, X, Y, Z, AA, BB, CC5 and CC6, wherein the plurality of pathogens or infectious agents are HIV-1, HIV-2, HCV, HBV, Chikungunya Virus, Zika Virus, and WNV.

EE36. The system of any one of U-U6, V, W, X, Y, Z, AA, BB, CC5 and CC6, wherein the plurality of pathogens or infectious agents are HIV-1, HIV-2, HCV, HBV, WNV, and Dengue Virus.

EE37. The system of any one of U-U6, V, W, X, Y, Z, AA, BB, CC5 and CC6, wherein the plurality of pathogens or infectious agents are HIV-1, HIV-2, HCV, HBV, Chikungunya Virus, Zika Virus, and WNV.

EE38. The system of any one of U-U6, V, W, X, Y, Z, AA, BB, CC5 and CC6, wherein the plurality of pathogens or infectious agents are HIV-1, HIV-2, HCV, and HBV; and the nucleic acid analysis comprises multiplex analysis of HIV-1 and HIV-2.

EE39. The system of any one of U-U6, V, W, X, Y, Z, AA, BB, CC5 and CC6, wherein the plurality of pathogens or infectious agents are HIV-1, HIV-2, HCV, and HBV; and the nucleic acid analysis comprises multiplex analysis of HIV-1, HIV-2, and HCV.

EE40. The system of any one of U-U6, V, W, X, Y, Z, AA, BB, CC5 and CC6, wherein the plurality of pathogens or infectious agents are HIV-1, HIV-2, HCV, and HBV; and the nucleic acid analysis comprises multiplex analysis of HIV-1, HIV-2, and HBV.

EE41. The system of any one of U-U6, V, W, X, Y, Z, AA, BB, CC5 and CC6, wherein the plurality of pathogens or infectious agents are HIV-1, HIV-2, HCV, and HBV; and the nucleic acid analysis comprises multiplex analysis of HCV, and HBV.

EE42. The system of any one of U-U6, V, W, X, Y, Z, AA, BB, CC5 and CC6, wherein the plurality of pathogens or infectious agents are HIV-1, HIV-2, HCV, and HBV; the nucleic acid analysis comprises multiplex analysis of HIV-1 and HIV-2; and the nucleic acid analysis comprises multiplex analysis of HCV and HBV.

EE43. The system of any one of U-U6, V, W, X, Y, Z, AA, BB, CC5 and CC6, wherein the plurality of pathogens or infectious agents are HIV-1, HIV-2, HCV, and HBV; and the nucleic acid analysis comprises multiplex analysis of HIV-1, HIV-2, HCV, and HBV.

EE44. The system of any one of U-U6, V, W, X, Y, Z, AA, BB, CC5 and CC6, wherein the plurality of pathogens or infectious agents comprise Zika Virus, WNV, Chikungunya Virus and Dengue Virus; the nucleic acid analysis comprises multiplex analysis of Zika Virus and WNV.

EE45. The system of any one of U-U6, V, W, X, Y, Z, AA, BB, CC5 and CC6, wherein the plurality of pathogens or infectious agents comprise Zika Virus, WNV, Chikungunya Virus and Dengue Virus; the nucleic acid analysis comprises multiplex analysis of Chikungunya Virus and Dengue Virus.

EE46. The system of any one of U-U6, V, W, X, Y, Z, AA, BB, CC5 and CC6, wherein the plurality of pathogens or infectious agents comprise Zika Virus, WNV, Chikungunya Virus and Dengue Virus; the nucleic acid analysis comprises multiplex analysis of Zika Virus and WNV; and the nucleic acid analysis comprises multiplex analysis of Chikungunya Virus and Dengue Virus.

EE47. The system of any one of U-U6, V, W, X, Y, Z, AA, BB, CC5 and CC6, wherein the plurality of pathogens or infectious agents comprise Zika Virus, WNV, Chikungunya Virus and Dengue Virus; the nucleic acid analysis comprises multiplex analysis of Chikungunya Virus and WNV; and the nucleic acid analysis comprises multiplex analysis of Zika Virus and Dengue Virus.

EE48. The system of any one of U-U6, V, W, X, Y, Z, AA, BB, CC5 and CC6, wherein the plurality of pathogens or infectious agents comprise Zika Virus, WNV, Chikungunya Virus and Dengue Virus; the nucleic acid analysis comprises multiplex analysis of Zika Virus and Dengue; and the nucleic acid analysis comprises multiplex analysis of Chikungunya Virus and WNV.

EE49. The system of any one of U-U6, V, W, X, Y, Z, AA, BB, CC5 and CC6, wherein the plurality of pathogens or infectious agents comprise Babesia and Malaria; and the nucleic acid analysis comprises multiplex analysis of Babesia and Malaria.

EE50. The system of any one of U-U6, V, W, X, Y, Z, AA, BB, CC5 and CC6, wherein the plurality of pathogens or infectious agents comprise Parvovirus B19 and HAV; and the nucleic acid analysis comprises multiplex analysis of Parvovirus B19 and HAV.

EE51. The system of any one of U-U6, V, W, X, Y, Z, AA, BB, and CC6, wherein the plurality of pathogens or infectious agents and predetermined levels are selected from the following: HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; CMV at a predetermined level of at least 10-50 IU/mL; Parvovirus B19 at a predetermined level of at least 1-40 IU/mL; HAV at a predetermined level of at least 1-10 IU/mL; Gonorrhea at a predetermined level of at least 100-500 copies/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Babesia at a predetermined level of at least 1-20 copies/mL; Malaria at a predetermined level of at least 1-50 copies/mL; and HEV at a predetermined level of at least 1-20 IU/mL.

EE52. The system of any one of U-U6, V, W, X, Y, Z, AA, BB, and CC6, wherein the plurality of pathogens or infectious agents are HIV-1, HIV-2, HCV, and HBV; and wherein the predetermined levels are at least 1-50 copies/mL of HIV-1; at least 1-20 IU/mL of HIV-2; at least 1-50 IU/mL of HCV; and at least 1-10 IU/mL of HBV.

EE53. The system of any one of U-U6, V, W, X, Y, Z, AA, BB, and CC6, wherein the plurality of pathogens or infectious agents are HIV-1, HIV-2, HCV, HBV, and WNV; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of WNV.

EE54. The system of any one of U-U6, V, W, X, Y, Z, AA, BB, and CC6, wherein the plurality of pathogens or infectious agents are Zika Virus and WNV; and wherein the predetermined levels are at least 1-50 copies/mL of Zika Virus; and at least 1-50 copies/mL of WNV.

EE55. The system of any one of U-U6, V, W, X, Y, Z, AA, BB, and CC6, wherein the plurality of pathogens or infectious agents are Chikungunya Virus and Dengue Virus; and wherein the predetermined levels are at least 1-50 copies/mL of Chikungunya Virus; and at least 1-50 copies/mL of Dengue Virus.

EE56. The system of any one of U-U6, V, W, X, Y, Z, AA, BB, and CC6, wherein the plurality of pathogens or infectious agents are Zika Virus, WNV, Chikungunya Virus and Dengue Virus; and wherein the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

EE57. The system of any one of U-U6, V, W, X, Y, Z, AA, BB, and CC6, wherein the plurality of pathogens or infectious agents are Babesia and Malaria; and wherein the predetermined levels are at least 1-20 copies/mL of Babesia; and at least 1-50 copies/mL of Malaria.

EE58. The system of any one of U-U6, V, W, X, Y, Z, AA, BB, and CC6, wherein the plurality of pathogens or infectious agents are Parvovirus B19 and HAV; and wherein the predetermined levels are at least 1-40 IU/mL of Parvovirus B19; and at least 1-10 IU/mL of HAV.

EE59. The system of any one of U-U6, V, W, X, Y, Z, AA, BB, and CC6, wherein the plurality of pathogens or infectious agents are HIV-1, HIV-2, HCV, HBV, WNV, and Zika Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Zika Virus.

EE60. The system of any one of U-U6, V, W, X, Y, Z, AA, BB, and CC6, wherein the plurality of pathogens or infectious agents are HIV-1, HIV-2, HCV, HBV, WNV, Zika Virus, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; and at least 1-50 copies/mL of Chikungunya Virus.

EE61. The system of any one of U-U6, V, W, X, Y, Z, AA, BB, and CC6, wherein the plurality of pathogens or infectious agents are HIV-1, HIV-2, HCV, HBV, WNV, Zika Virus, Chikungunya Virus, and Dengue Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; and at least 1-50 copies/mL of Dengue Virus.

EE62. The system of any one of U-U6, V, W, X, Y, Z, AA, BB, and CC6, wherein the plurality of pathogens or infectious agents are HIV-1, HIV-2, HCV, HBV, WNV, Zika Virus, Chikungunya Virus, Dengue Virus, and Babesia; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; and at least 1-20 copies/mL of Babesia.

EE62. The system of any one of U-U6, V, W, X, Y, Z, AA, BB, and CC6, wherein the plurality of pathogens or infectious agents are HIV-1, HIV-2, HCV, HBV, WNV, Zika Virus, Chikungunya Virus, Dengue Virus, Babesia, and Malaria; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria.

EE63. The system of any one of U-U6, V, W, X, Y, Z, AA, BB, and CC6, wherein the plurality of pathogens or infectious agents are HIV-1, HIV-2, HCV, HBV, WNV, Zika Virus, Chikungunya Virus, Dengue Virus, Babesia, Malaria, and Parvovirus B19; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; and at least 1-40 IU/mL of Parvovirus B19.

EE64. The system of any one of U-U6, V, W, X, Y, Z, AA, BB, and CC6, wherein the plurality of pathogens or infectious agents are HIV-1, HIV-2, HCV, HBV, WNV, Zika Virus, Chikungunya Virus, Dengue Virus, Babesia, Malaria, Parvovirus B19, and HAV; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; and at least 1-10 IU/mL of HAV.

EE65. The system of any one of U-U6, V, W, X, Y, Z, AA, BB, and CC6, wherein the plurality of pathogens or infectious agents are HIV-1, HIV-2, HCV, HBV, WNV, Zika Virus, Chikungunya Virus, Dengue Virus, Babesia, Malaria, Parvovirus B19, HAV, and HEV; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; at least 1-50 copies/mL of Zika Virus; at least 1-50 copies/mL of Chikungunya Virus; at least 1-50 copies/mL of Dengue Virus; at least 1-20 copies/mL of Babesia; and at least 1-50 copies/mL of Malaria; at least 1-40 IU/mL of Parvovirus B19; at least 1-10 IU/mL of HAV; and at least 1-20 IU/mL of HEV.

EE66. The system of any one of U-U6, V, W, X, Y, Z, AA, BB, and CC6, wherein the plurality of pathogens or infectious agents are HIV-1, HIV-2, HCV, HBV, and Parvovirus B19; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-40 IU/mL of Parvovirus B19.

EE67. The system of any one of U-U6, V, W, X, Y, Z, AA, BB, and CC6, wherein the plurality of pathogens or infectious agents are HIV-1, HIV-2, HCV, HBV, Parvovirus B19, and HAV; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-40 IU/mL of Parvovirus B19; and at least 1-10 IU/mL of HAV.

EE68. The system of any one of U-U6, V, W, X, Y, Z, AA, BB, and CC6, wherein the plurality of pathogens or infectious agents are HIV-1, HIV-2, HCV, HBV, and Babesia; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-20 copies/mL of Babesia.

EE69. The system of any one of U-U6, V, W, X, Y, Z, AA, BB, and CC6, wherein the plurality of pathogens or infectious agents are HIV-1, HIV-2, HCV, HBV, and HAV; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-10 IU/mL of HAV.

EE70. The system of any one of U-U6, V, W, X, Y, Z, AA, BB, and CC6, wherein the plurality of pathogens or infectious agents are HIV-1, HIV-2, HCV, HBV, and HEV; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-20 IU/mL HEV.

EE71. The system of any one of U-U6, V, W, X, Y, Z, AA, BB, and CC6, wherein the plurality of pathogens or infectious agents are HIV-1, HIV-2, HCV, HBV, and Zika Virus; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Zika Virus.

EE72. The system of any one of U-U6, V, W, X, Y, Z, AA, BB, and CC6, wherein the plurality of pathogens or infectious agents are HIV-1, HIV-2, HCV, HBV, Zika Virus, and Dengue Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; and at least 1-50 copies/mL of Dengue Virus.

EE73. The system of any one of U-U6, V, W, X, Y, Z, AA, BB, and CC6, wherein the plurality of pathogens or infectious agents are HIV-1, HIV-2, HCV, HBV, Zika Virus, Dengue Virus, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of Chikungunya Virus.

EE74. The system of any one of U-U6, V, W, X, Y, Z, AA, BB, and CC6, wherein the plurality of pathogens or infectious agents are HIV-1, HIV-2, HCV, HBV, Zika Virus, Dengue Virus, and WNV; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of Dengue Virus; and at least 1-50 copies/mL of WNV.

EE75. The system of any one of U-U6, V, W, X, Y, Z, AA, BB, and CC6, wherein the plurality of pathogens or infectious agents are HIV-1, HIV-2, HCV, HBV, Zika Virus, WNV, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; and at least 1-50 copies/mL of Chikungunya Virus.

EE76. The system of any one of U-U6, V, W, X, Y, Z, AA, BB, and CC6, wherein the plurality of pathogens or infectious agents are HIV-1, HIV-2, HCV, HBV, Zika Virus, WNV, Dengue Virus, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Zika Virus; at least 1-50 copies/mL of WNV; at least 1-50 copies/mL of Dengue Virus; and at least 1-50 copies/mL of Chikungunya Virus.

EE77. The system of any one of U-U6, V, W, X, Y, Z, AA, BB, and CC6, wherein the plurality of pathogens or infectious agents are HIV-1, HIV-2, HCV, HBV, and Malaria; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Malaria.

EE78. The system of any one of U-U6, V, W, X, Y, Z, AA, BB, and CC6, wherein the plurality of pathogens or infectious agents are HIV-1, HIV-2, HCV, HBV, Malaria, and Babesia; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Malaria; and at least 1-20 copies/mL of Babesia.

EE79. The system of any one of U-U6, V, W, X, Y, Z, AA, BB, and CC6, wherein the plurality of pathogens or infectious agents are HIV-1, HIV-2, HCV, HBV, and Dengue Virus; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Dengue Virus.

EE80. The system of any one of U-U6, V, W, X, Y, Z, AA, BB, and CC6, wherein the plurality of pathogens or infectious agents are HIV-1, HIV-2, HCV, HBV, Dengue Virus, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Dengue Virus; and at least 1-50 copies/mL of Chikungunya Virus.

EE81. The system of any one of U-U6, V, W, X, Y, Z, AA, BB, and CC6, wherein the plurality of pathogens or infectious agents are HIV-1, HIV-2, HCV, HBV, Dengue Virus, WNV, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL Dengue Virus; at least 1-50 copies/mL of WNV; and at least 1-50 copies/mL of Chikungunya Virus.

EE82. The system of any one of U-U6, V, W, X, Y, Z, AA, BB, and CC6, wherein the plurality of pathogens or infectious agents are HIV-1, HIV-2, HCV, HBV, and Chikungunya Virus; and wherein the predetermined levels are at least 1-50 copies/mL of HIV-1; at least 1-20 IU/mL of HIV-2; at least 1-50 IU/mL of HCV; at least 1-10 IU/mL of HBV; and at least 1-50 copies/mL of Chikungunya Virus.

EE83. The system of any one of U-U6, V, W, X, Y, Z, AA, BB, and CC6, wherein the plurality of pathogens or infectious agents are HIV-1, HIV-2, HCV, HBV, Chikungunya Virus, and Zika Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus; and at least 1-50 copies/mL of Zika Virus.

EE84. The system of any one of U-U6, V, W, X, Y, Z, AA, BB, and CC6, wherein the plurality of pathogens or infectious agents are HIV-1, HIV-2, HCV, HBV, Chikungunya Virus, Zika Virus, and WNV; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of Chikungunya Virus at least 1-50 copies/mL of Zika Virus; and at least 1-50 copies/mL of WNV.

EE85. The system of any one of U-U6, V, W, X, Y, Z, AA, BB, and CC6, wherein the plurality of pathogens or infectious agents are HIV-1, HIV-2, HCV, HBV, WNV, and Dengue Virus; and wherein the predetermined levels are at least 1-50 copies/mL of HIV-1; at least 1-20 IU/mL of HIV-2; at least 1-50 IU/mL of HCV; at least 1-10 IU/mL of HBV; at least 1-50 copies/mL of WNV; and at least 1-50 copies/mL of Dengue Virus.

EE86. The system of any one of U-U6, V, W, X, Y, Z, AA, BB, and CC6, wherein the plurality of pathogens or infectious agents are HIV-1, HIV-2, HCV, and HBV; the nucleic acid analysis comprises multiplex analysis of HIV-1 and HIV-2; and the predetermined levels are: at least 1-50 copies/mL of HIV-1; at least 1-20 IU/mL of HIV-2; at least 1-50 IU/mL of HCV; and at least 1-10 IU/mL of HBV.

EE87. The system of any one of U-U6, V, W, X, Y, Z, AA, BB, and CC6, wherein the plurality of pathogens or infectious agents are HIV-1, HIV-2, HCV, and HBV; the nucleic acid analysis comprises multiplex analysis of HIV-1, HIV-2, and HCV; and the predetermined levels are: at least 1-50 copies/mL of HIV-1; at least 1-20 IU/mL of HIV-2; at least 1-50 IU/mL of HCV; and at least 1-10 IU/mL of HBV.

EE88. The system of any one of U-U6, V, W, X, Y, Z, AA, BB, and CC6, wherein the plurality of pathogens or infectious agents are HIV-1, HIV-2, HCV, and HBV; the nucleic acid analysis comprises multiplex analysis of HIV-1, HIV-2, and HBV; and the predetermined levels are at least 1-50 copies/mL of HIV-1; at least 1-20 IU/mL of HIV-2; at least 1-50 IU/mL of HCV; and at least 1-10 IU/mL of HBV.

EE89. The system of any one of U-U6, V, W, X, Y, Z, AA, BB, and CC6, wherein the plurality of pathogens or infectious agents are HIV-1, HIV-2, HCV, and HBV; the nucleic acid analysis comprises multiplex analysis of HCV, and HBV; and the predetermined levels are at least 1-50 copies/mL of HIV-1; at least 1-20 IU/mL of HIV-2; at least 1-50 IU/mL of HCV; and at least 1-10 IU/mL of HBV.

EE90. The system of any one of U-U6, V, W, X, Y, Z, AA, BB, and CC6, wherein the plurality of pathogens or infectious agents are HIV-1, HIV-2, HCV, and HBV; the nucleic acid analysis comprises multiplex analysis of HIV-1 and HIV-2; the nucleic acid analysis comprises multiplex analysis of HCV and HBV; and the predetermined levels are at least 1-50 copies/mL of HIV-1; at least 1-20 IU/mL of HIV-2; at least 1-50 IU/mL of HCV; and at least 1-10 IU/mL of HBV.

EE91. The system of any one of U-U6, V, W, X, Y, Z, AA, BB, and CC6, wherein the plurality of pathogens or infectious agents are HIV-1, HIV-2, HCV, and HBV; the nucleic acid analysis comprises multiplex analysis of HIV-1, HIV-2, HCV, and HBV; and the predetermined levels are at least 1-50 copies/mL of HIV-1; at least 1-20 IU/mL of HIV-2; at least 1-50 IU/mL of HCV; and at least 1-10 IU/mL of HBV.

EE92. The system of any one of U-U6, V, W, X, Y, Z, AA, BB, and CC6, wherein the plurality of pathogens or infectious agents comprise Zika Virus, WNV, Chikungunya Virus and Dengue Virus; the nucleic acid analysis comprises multiplex analysis of Zika Virus and WNV; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

EE93. The system of any one of U-U6, V, W, X, Y, Z, AA, BB, and CC6, wherein the plurality of pathogens or infectious agents comprise Zika Virus, WNV, Chikungunya Virus and Dengue Virus; the nucleic acid analysis comprises multiplex analysis of Chikungunya Virus and Dengue Virus; and the predetermined levels are: at least 1-50 copies/mL of Zika Virus; at least 1-50 copies/mL of WNV; at least 1-50 copies/mL of Chikungunya Virus; and at least 1-50 copies/mL of Dengue Virus.

EE94. The system of any one of U-U6, V, W, X, Y, Z, AA, BB, and CC6, wherein the plurality of pathogens or infectious agents comprise Zika Virus, WNV, Chikungunya Virus and Dengue Virus; the nucleic acid analysis comprises multiplex analysis of Zika Virus and WNV; the nucleic acid analysis comprises multiplex analysis of Chikungunya Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

EE95. The system of any one of U-U6, V, W, X, Y, Z, AA, BB, and CC6, wherein the plurality of pathogens or infectious agents comprise Zika Virus, WNV, Chikungunya Virus and Dengue Virus; the nucleic acid analysis comprises multiplex analysis of Chikungunya Virus and WNV; the nucleic acid analysis comprises multiplex analysis of Zika Virus and Dengue Virus; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

EE96. The system of any one of U-U6, V, W, X, Y, Z, AA, BB, and CC6, wherein the plurality of pathogens or infectious agents comprise Zika Virus, WNV, Chikungunya Virus and Dengue Virus; the nucleic acid analysis comprises multiplex analysis of Zika Virus and Dengue; the nucleic acid analysis comprises multiplex analysis of Chikungunya Virus and WNV; and the predetermined levels are at least 1-50 copies/mL of Zika Virus; at least 1-50 copies/mL of WNV; at least 1-50 copies/mL of Chikungunya Virus; and at least 1-50 copies/mL of Dengue Virus.

EE97. The system of any one of U-U6, V, W, X, Y, Z, AA, BB, and CC6, wherein the plurality of pathogens or infectious agents comprise Babesia and Malaria; the nucleic acid analysis comprises multiplex analysis of Babesia and Malaria; and the predetermined levels are at least 1-50 copies/mL of Malaria; and at least 1-20 copies/mL of Babesia.

EE98. The system of any one of U-U6, V, W, X, Y, Z, AA, BB, and CC6, wherein the plurality of pathogens or infectious agents comprise Parvovirus B19 and HAV; the nucleic acid analysis comprises multiplex analysis of Parvovirus B19 and HAV; and the predetermined levels are at least 1-40 IU/mL of Parvovirus B19; and at least 1-10 IU/mL of HAV.

EE99. The system of any one of CC-CC6 and EE-EE6, wherein the sample or one or more samples is a sample of donor blood.

EE100. The system of any one of U-U6, V, W, X, Y, Z, AA, BB, and EE99, wherein the sample of donor blood is human donor blood.

EE101. The system of any one of U-U6, V, W, X, Y, Z, AA, BB, and EE99, wherein the sample of donor blood is whole blood.

EE102. The system of any one of U-U6, V, W, X, Y, Z, AA, BB, and EE99, wherein the sample of donor blood is lysed whole blood.

EE103. The system of any one of U-U6, V, W, X, Y, Z, AA, BB, and EE99, wherein the sample of donor blood is serum.

EE104. The system of any one of U-U6, V, W, X, Y, Z, AA, BB, and EE99, wherein the sample of donor blood is plasma.

EE105. The system of any one of U-U6, V, W, X, Y, Z, AA, BB, and EE7-EE104, wherein the release of the donor material is for transfusion.

EE106. The system of any one of U-U6, V, W, X, Y, Z, AA, BB, and EE7-EE104, wherein the release of the donor material is for use in a pharmaceutical.

EE107. The system of any one of U-U6, V, W, X, Y, Z, AA, BB, and EE7-EE104, wherein the release of the donor material is for use in a therapeutic treatment.

EE108. The system of any one of U-U6, V, W, X, Y, Z, AA, BB, and EE7-EE104, wherein the sample of donor blood is for use as a blood donation.

EE109. The system of any one of U-U6, V, W, X, Y, Z, AA, BB, and EE7-EE104, wherein the clinical use is an in vivo clinical use.

EE110. The system of any one of U-U6, V, W, X, Y, Z, AA, BB, and EE7-EE104, wherein the clinical use is an in vitro clinical use.

EE111. The system of any one of U-U6, V, W, X, Y, Z, AA, BB, EE7-EE104, and EE109, wherein the clinical use is transfusion.

EE112. The system of any one of U-U6, V, W, X, Y, Z, AA, BB, EE7-EE104, and EE109, wherein the clinical use is use in a pharmaceutical.

EE113. The system of any one of U-U6, V, W, X, Y, Z, AA, BB, EE7-EE104, and EE109, wherein the clinical use is use in a therapeutic treatment.

EE114. The system of any one of U-U6, V, W, X, Y, Z, AA, BB, EE7-EE104, and EE109, wherein the clinical use is organ or tissue donation.

EE115. The system of any one of U-U6, V, W, X, Y, Z, AA, BB, EE7-EE104, and EE109, wherein the clinical use is use in a plasma-derived product.

EE116. The system of any one of U-U6, V, W, X, Y, Z, AA, BB, EE7-EE104, and EE110, wherein the clinical use is a diagnostic use.

EE117. The system of any one of U-EE116, wherein the nucleic acid analysis comprises a nucleic acid amplification reaction.

EE118. The system of EE117, wherein the nucleic acid amplification reaction is an isothermal reaction.

EE119. The system of E118, wherein the isothermal reaction is recombinase polymerase amplification.

EE120. The system of E118, wherein the isothermal reaction is a nicking enzyme amplification reaction.

EE121. The system of any one of U-EE120, wherein the nucleic acid analysis comprises digital detection of the presence of a nucleic acid derived from at least one of a plurality of pathogens or infectious agents.

EE122. The system of EE121, wherein the system is configured to digitally detect the presence of a nucleic acid derived from at least one of the plurality of pathogens or infectious agents a plurality of times to quantitatively detect the presence of a nucleic acid derived from at least one of the plurality of pathogens or infectious agents.

EE123. The system of any one of U-EE120, wherein the nucleic acid analysis comprises optical detection of the presence of a nucleic acid derived from at least one of a plurality of pathogens or infectious agents.

EE124. The system of EE123, wherein the system is configured to optically detect the presence of a nucleic acid derived from at least one of the plurality of pathogens or infectious agents a plurality of times to quantitatively detect the presence of a nucleic acid derived from at least one of the plurality of pathogens or infectious agents.

EE125. The system of any one of U-EE124, wherein the nucleic acid analysis comprises contemporaneous contact of the sample with sample lysis buffer and a protease.

EE126. The system of any one of U-EE125, wherein the nucleic acid analysis comprises contacting the sample with CuTi-coated microparticles.

EE127. The system of any one of U-EE126, wherein the nucleic acid analysis comprises contacting the sample with a plurality of microparticles and translation of the microparticles on a surface via magnetic force.

EE128. The system of any one of U-EE127, wherein the nucleic acid analysis comprises purification of nucleic acid from the sample of donor blood; division of the purified nucleic acid into a plurality of fractions; and at least one fraction is reserved for further screening.

EE129. The system of any one of U-EE128, wherein the nucleic acid analysis is completed in about 15 to about 36 minutes.

EE130. The system of any one of U-EE128, wherein the nucleic acid analysis is completed in about 32 to about 36 minutes.

EE131. The system of any one of U-EE130, wherein the nucleic acid analysis comprises a sample preparation process at the sample preparation area and an amplification and detection process at an amplification and detection system.

EE132. The system of EE131, wherein the sample preparation process comprises a lysis process and a wash process.

EE133. The system of any one of EE131-EE132, wherein the sample preparation process is completed in about 12 to about 16 minutes.

EE134. The system of any one of EE131-EE133, wherein the amplification and detection process is completed in about 1 to about 22 minutes.

EE135. The system of any one of EE131-EE134, wherein the amplification and detection process is completed in about 5 to about 22 minutes.

EE136. The system of any one of EE131-EE135, wherein the amplification and detection process is completed in about 18 to about 22 minutes.

EE137. The system of any one of EE131-EE136, wherein the lysis process is completed in about 4 to about 8 minutes.

EE138. The system of any one of EE131-EE137, wherein the wash process is completed in about 5 to about 9 minutes.

EE139. The system of any one of EE131-EE138, wherein the wash process comprises a first wash step, a second wash step, a third wash step and an elution step.

EE140. The system of EE139, wherein the first wash step is completed in about 1 to about 3 minutes.

EE141. The system of EE139 or EE140, wherein the second wash step is completed in about 0.1 to about 1 minute.

EE142. The system of any one of EE139-EE141, wherein the third wash step is completed in about 0.1 to about 1 minute.

EE143. The system of any one of EE139-EE142, wherein the elution step is completed in about 2 to about 5 minutes.

EE144. The system of any one of EE139-EE143, wherein the sample preparation further comprises a pre-treatment process.

EE145. The system of EE144, wherein the pre-treatment process is completed in about 3 to about 7 minutes.

EE146. The system of EE144 or EE145, wherein the sample preparation process further comprises an onboard pooling process, and wherein up to 6 samples of donor blood are pre-treated and pooled in about 6 minutes or less.

EE147. The system of any one of EE131-EE143, wherein the sample preparation process further comprises an onboard pooling process.

EE148. The system of EE147, wherein up to 24 samples are pooled in about 5 minutes or less.

EE149. The system of any of U-EE148, wherein the nucleic acid analysis includes at least about 140 sample aspirations per hour.

EE150. The system of any of U-EE149, wherein the nucleic acid analysis includes an amplification process including at least about 140 amplifications per hour.

EE151. The system of any of U-EE150, wherein the nucleic acid analysis includes an amplification process including multiplex amplification.

EE152. The system of any of U-EE151, wherein the nucleic acid analysis includes a sample preparation process including preparing eluate and wherein the eluate is split into two or more amplification vessels.

EE153. The system of any of U-EE152, wherein between about 140 results and about 300 results are obtained per hour per m² of a footprint of the automated system.

EE154. The system of U-CC6, and EE-EE153, wherein the sample of donor blood, sample, or one or more samples is a pooled sample.

EE155. The system of EE154, wherein the pooled sample comprises blood or plasma or serum or biological samples from 2 donors.

EE156. The system of EE154, wherein the pooled sample of donor blood comprises blood or plasma or serum or biological samples from 3 donors.

EE157. The system of EE154, wherein the pooled sample of donor blood comprises blood or plasma or serum or biological samples from 4 donors.

EE158. The system of EE154, wherein the pooled sample of donor blood comprises blood or plasma or serum or biological samples from 5 donors.

EE159. The system of EE154, wherein the pooled sample of donor blood comprises blood or plasma or serum or biological samples from 10 donors.

EE160. The system of EE154, wherein the pooled sample of donor blood comprises blood or plasma or serum or biological samples from 12 donors EE161. The system of EE154, wherein the pooled sample of donor blood comprises blood or plasma or serum or biological samples from 96 donors.

EE162. The system of any of BB53-BB60, wherein the nucleic acid analysis includes a sample preparation process comprising onboard pooling of a subset of a plurality of samples at the sample preparation area to form a pooled sample, the nucleic acid analysis being performed on the pooled sample EE163. The system of EE162, wherein the sample preparation area includes at least one pipettor and at least one sample transport, and wherein the sample transport is configured to continually transport individual vessels along a transport path from a sample dispense position to a sample capture and transfer position, with intermediate positions therebetween; and wherein the onboard pooling includes dispensing a first sample and a second sample in a vessel on the sample transport to form a pooled sample.

EE164. The system of EE163, wherein the sample transport includes a serpentine path, carousel, conveyor, extrusion, robotic handler, or belt.

EE165. The system of any of EE163 of EE164, wherein the sample transport includes a sample preparation carousel and wherein the sample preparation carousel is configured to rotate to continually transport individual vessels along the transport path.

EE166. The system of EE165, wherein the sample preparation carousel is configured to rotate in a lockstep fashion, moving one position with each lockstep.

EE167. The system of EE166, wherein the duration of each lockstep is between about 15 and about 30 seconds.

EE168. The system of any of EE162-EE167, wherein the system is configured to hold samples included in the pooled sample at the sample loading area during nucleic acid analysis of the pooled sample.

EE169. The system of any of EE162-EE168, wherein upon a determination of the presence of a nucleic acid derived from at least one of a plurality of pathogens or infectious agents in excess of a predetermined level in the pooled sample, the instructions, when executed by the processor, cause the system to perform nucleic acid analysis of individual samples or sub-pools thereof included in the pooled sample; and wherein upon a determination of a predetermined level of nucleic acids derived from each of the plurality of pathogens or infectious agents based on the nucleic acid analysis of the individual samples or sub-pools thereof, the system is indicative of release of a donor material associated with the individual samples or sub-pools thereof for clinical use.

EE170. The system of any one of CC-CC6, wherein the pathogen or infectious agent is selected from the group consisting of: SARS-CoV-2 (COVID-19), coronaviruses, HIV-1, HIV-2, HBV, HCV, CMV, Epstein-Barr virus (EBV), human T-lymphotropic virus (HTLV), Parvovirus B19, HAV, syphilis, Chlamydia, Gonorrhea, Dengue, Chikungunya, WNV, HEV, Creutzfeldt-Jakob disease (vCJD) and a combination thereof.

EE180. The method of EE170, wherein the plurality of pathogens or infectious agents and predetermined levels are selected from the following SARS-CoV-2 (COVID-19) at a predetermined level of at least 1-50 copies/mL; HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; CMV at a predetermined level of at least 10-50 IU/mL; Parvovirus B19 at a predetermined level of at least 1-40 IU/mL; HAV at a predetermined level of at least 1-10 IU/mL; Chlamydia at a predetermined level of at least 100-500 copies/mL; Gonorrhea at a predetermined level of at least 100-500 copies/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Influenza at a predetermined level of at least 10-500 copies/mL; Babesia at a predetermined level of at least 1-20 copies/mL; Malaria at a predetermined level of at least 1-50 copies/mL; and HEV at a predetermined level of at least 1-20 IU/mL.

EE171. The system of any one of CC-CC6, wherein the plurality of pathogens or infectious agents and predetermined levels are selected from the following: SARS-CoV-2 (COVID-19) at a predetermined level of at least 1-50 copies/mL; HIV-1 at a predetermined level of at least 1-50 copies/mL; HIV-2 at a predetermined level of at least 1-20 IU/mL; HBV at a predetermined level of at least 1-10 IU/mL; HCV at a predetermined level of at least 1-50 IU/mL; CMV at a predetermined level of at least 10-50 IU/mL; Parvovirus B19 at a predetermined level of at least 1-40 IU/mL; HAV at a predetermined level of at least 1-10 IU/mL; Chlamydia at a predetermined level of at least 100-500 copies/mL; Gonorrhea at a predetermined level of at least 100-500 copies/mL; WNV at a predetermined level of at least 1-50 copies/mL; Zika Virus at a predetermined level of at least 1-50 copies/mL; Dengue Virus at a predetermined level of at least 1-50 copies/mL; Chikungunya Virus at a predetermined level of at least 1-50 copies/mL; Influenza at a predetermined level of at least 10-500 copies/mL; Babesia at a predetermined level of at least 1-20 copies/mL; Malaria at a predetermined level of at least 1-50 copies/mL; and HEV at a predetermined level of at least 1-20 IU/mL.

While the disclosed subject matter is described herein in terms of certain embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the methods and systems of the disclosed subject matter without departing from the spirit or scope of the disclosed subject matter. Thus, it is intended that the disclosed subject matter include modifications and variations that are within the scope of the appended claims and their equivalents.

### EXAMPLES

### Example 1: Nucleic Acid Testing of Samples <60 Min Per Assay

FIG. 1 illustrates an exemplary HTNAT sample analysis process according to the disclosed subject matter. This exemplary HTNAT sample analysis process comprises three operations: sample preparation, amplification, and detection.

FIG. 2 is a diagram illustrating an exemplary HTNAT sample analysis process according to the disclosed subject matter comprising three operations: sample preparation process (including pre-treatment lysis process, sample lysis process, and wash & elute process), amplification process (including reagent addition operations), and detection/reading process. Exemplary strategies are further illustrated in FIG. 2B, where assay-specific steps (e.g., pre-treatment lysis process of whole blood with an RBC lysis buffer, to produce a lysed whole blood sample), lead to a unified sample preparation process path comprising exposure to a sample lysis buffer followed by multiple washes and elution at 80° C. in 5 mM PO₄.

FIG. 3 is a diagram illustrating an exemplary HTNAT automated sample analysis system (1) according to the disclosed subject matter comprising elements for accomplishing the three processes outlined in FIG. 2, specifically: sample preparation (2) (i.e., pre-lysis (4), sample lysis (5), and wash (6) & elute (7)), amplification and detection/reading (3).

FIG. 4 is a diagram illustrating exemplary embodiments of the sample preparation process (direct capture and CuTi target nucleic acid (“TNA”) capture) in accordance with the disclosed subject matter.

### Example 2. Sample Preparation Incorporating Stationary Electromagnet-Based

### Capture and Wash of Magnetic Particles

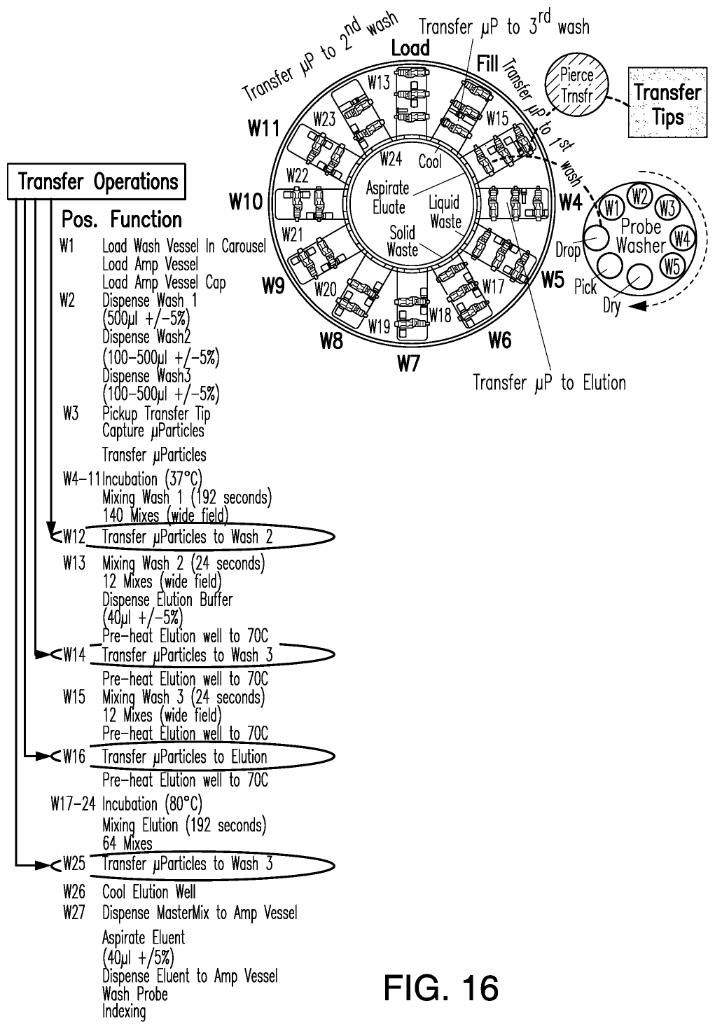

Each of the embodiments described in Example 1 can be performed by incorporating at least one step involving a stationary electromagnet-based capture of magnetic particles. Operational steps involving a stationary electromagnet-based capture of magnetic particles include but are not limited to: mixing; washing; and transfer of the magnetic particles. Existing methods of mixing and washing magnetic particles generally rely on mechanical agitation and magnetic mixing using moving permanent magnets. Similarly, existing methods of transferring magnetic particles between wells generally include: capturing the magnetic particles on a sleeve-covered magnet and moving to another well; capturing the magnetic particles at the top of the liquid in a well, on the underside of a moving cover and moving to another well; capturing the magnetic particles at the top of the liquid in a well, on the underside of a stationary cover and moving to another well; and moving magnetic particles between the bottoms of two adjacent wells using a hydraulic lock. As outlined in FIG. 15, however, certain embodiments described herein can be performed with a system incorporating at least one stationary electromagnet-based capture of magnetic particles. For example, but not by way of limitation, one or more of the eighteen (18) distinct mixing or washing positions identified in FIG. 15 can be accomplished using a stationary electromagnet-based capture of magnetic particles. In addition, as outlined in FIG. 16, the embodiments described herein can be performed with a system incorporating at least one stationary electromagnet-based capture of magnetic particles in connection with one or more transfer operations at one or more of the four (4) pairs of distinct pairs of transfer positions. As will be appreciated by those of skill in the art, the specific number, orientation, and operational assignment (e.g., mixing, washing, transfer, or elution) of the individual positions identified in FIG. 15 and FIG. 16 can be modified as desired and yet remain within the scope of the present disclosure.

The present disclosure contemplates the use of stationary electromagnets in a variety of positions to accomplish the appropriate mixing, washing, and transfer operations. For example, but not by way of limitation, electromagnets can be positioned at right side, left side, and/or bottom side of a well. In addition, electromagnets can be positioned with one side higher than the other. The stationary electromagnets of the present disclosure can also be used in conjunction with a variety of well formats. FIG. 17 depicts an exemplary well format for use in connection with the methods and systems of the instant disclosure. Additional wells can easily be added to the design depicted in FIG. 17 and volume changes can easily be accommodated.

In certain embodiments, opposing electromagnets can be arranged to alternately attract magnetic particles to the opposite sides of a wash well. However, the position, timing, power, and sequence of the electromagnet activations is entirely flexible. For example, but not by way of limitation, stationary electromagnets and be used to partially collect magnetic particles and then allow them to drop to the bottom of the well. In certain embodiments, e.g., as exemplified in FIGS. 42A-42D, magnetic microparticles 4202 can be present in a first vessel of a plurality of vessels 4206. Alternatively, or additionally, an electromagnet 4204 can be used to capture the magnetic microparticles to the wall of the vessel 4206, as illustrated in FIG. 42B. Alternatively, or additionally, movement of the electromagnet 4204 to a second vessel induces the transfer of the magnetic microparticles 4202 from the first vessel to the second vessel, as illustrated in FIG. 42C. Alternatively, or additionally, elimination of the current to the electromagnet 4204 once the magnetic microparticles 4202 have transferred to the second vessel allows for release of the magnetic microparticles 4202 as illustrated in FIG. 42D. Alternatively or additionally, magnetic particles can be completely collected alternately on the sides of a well to facilitate mixing, washing, and/or transfer. In addition, magnetic particles can be slowly collected with lower power or magnetic particles can be more rapidly collected with higher power.

Distinct types of electromagnets are also contemplated for use in connection with the embodiments disclosed herein. For example, while DC electromagnets can be used, because DC electromagnets can magnetize magnetic particles, AC electromagnets can also be used in conjunction with or in place of DC electromagnets to avoid creating residual magnetism of the magnetic particles by varying the switching frequency.

The use of electromagnets in lieu of permanent magnets (or other mixing, washing, or transfer strategies) provides several advantages. Mixing or transfer operations during sample preparation typically requires permanent magnets to be moved in and out of range of the magnetic particles. The use of electromagnets can eliminate motion mechanisms by simply turning on and off the electromagnets. In addition, magnetic particles can be moved from one well to another by successively turning on and off adjacent magnets within an array. Incorporating a stationary electromagnet-based particle capture approach can also eliminate one or more disposable per test, thus reducing the amount of solid waste being generated. The use of stationary electromagnets also eliminates the need for specific volume requirements and disposable coverings for the wells or moving permanent magnet when transferring magnetic particles from one well to another. The instant strategy can also transfer magnetic particles between wells using moving electromagnets adjacent to the side of the wash vessel, but not touching the magnetic particles or liquid within wells, minimizing magnetic particle loss and liquid carryover from source to destination wells, thus maximizing assay performance.

An assessment of particle loss associated with an exemplary stationary electromagnet-based particle wash and transfer mechanism and disposable “side slide” well (depicted in FIG. 17) illustrates the excellent performance capabilities of the methods and systems disclosed herein. Table 10, below, provides the percentage of particles lost over the course of two sample preparation runs. Less than 5% loss of the particles is preferred and minimizes impact to assay performance, while less than 2% loss is excellent and maximizes assay performance.

TABLE 10

| | % Lost | | | | | | | |
|---|---|---|---|---|---|---|---|---|
| | | | | | | | | Overall |
| | | | | | | | | % |
| | | Transfer | | | | | | Particle |
| | Lysis | Tip | Wash 1 | Wash 2 | Wash 3 | Walls | Elution | Lost |
| Run 1 | 0.33% | 0.14% | 0.56% | 0.00% | 0.00% | 0.26% | 98.71% | 1.29% |
| Run 2 | 0.36% | 0.24% | 0.42% | 0.00% | 0.00% | 0.29% | 98.68% | 1.32% |
| Average | 0.35% | 0.19% | 0.49% | 0.00% | 0.00% | 0.28% | 98.70% | 1.30% |

An assessment of liquid carryover associated with an exemplary stationary electromagnet-based particle wash and transfer mechanism and disposable “side slide” well (depicted in FIG. 17) illustrates the excellent performance capabilities of the methods and systems disclosed herein. Table 11, below, provides liquid carryover (in this case, Guanidine Isothiocyanate GITC) from the source well—Wash1 to the destination well—Wash2, and subsequent destination well—Wash3. Less than 15 mM is preferred and minimizes impact to test performance, while less than 0.5 mM which is well under what amplification reactions (e.g., PCR and RPA) can tolerate.

TABLE 11

| | | | | Eluate- |
|---|---|---|---|---|
| | Wash1- | Wash2- | Wash3- | GITC mM |
| | GITC mM | GITC mM | GITC mM | (mean) |
| Run 1 | 4029 | 18.01 | 0.15* | N/A* |
| Run 2 | 4252 | 19.39 | 0.17* | N/A* |
| Average | 4029 | 18.70 | 0.16 | |

*Outside of the linear range for absorbance. Accuracy may be affected at this low a reading.

### Example 3: CuTi-Based Sample Preparation Implementation

FIG. 9 illustrates the implementation of an exemplary CuTi-based sample preparation process (the “−10 min” operation) relative to a conventional operation (the “−50 min” operation). In the −10 min operation, a sample (Plasma/Serum) is added together with Proteinase K, sample lysis buffer, and microparticles and incubated at 50-60° C. with agitation for about 5 minutes. Microparticles are captured via a sleeve magnet, e.g., a plunger, and transferred to a well containing sample lysis buffer and mixed for about 1 to about 2 minutes. Particles are then captured and transferred to a well containing water and mixed for about 24 to about 40 seconds to wash the particles. Multiple, e.g., two to three, of such washes are performed, resulting in about 2 minutes of washing. Particles are then delivered to elution buffer and heated to 80° C. for about 3 minutes to remove the nucleic acid from the solid phase. This eluate containing liquid is then used for a subsequent amplification reaction.

### Example 4: Direct Capture-Based Sample Preparation Implementation

FIG. 10 illustrates the implementation of a direct capture-based sample preparation process (the “−12 min” operation) relative to a conventional operation (the “−30 min” operation). In the −12 min operation, a sample, e.g., Plasma or Serum, is added together with Proteinase K, Sample lysis buffer, and specific capture oligonucleotides. The solution is incubated at 58° C. for about 1 minute followed by 95° C. for about 3 minutes and then 60° C. for about 3 minutes. Microparticles are then added and incubated at room temperature for about 2 minutes. Microparticles are captured via a magnet and transferred to a well containing water and mixed for about 1 minute (×2) to wash the microparticles. Particles are then delivered to elution buffer and heated to 80° C. for about 2 minutes to remove the nucleic acid from the solid phase. This eluate containing liquid is then used for a subsequent amplification reaction.

### Example 5: RPA-Based Amplification Implementation with Digital Detection

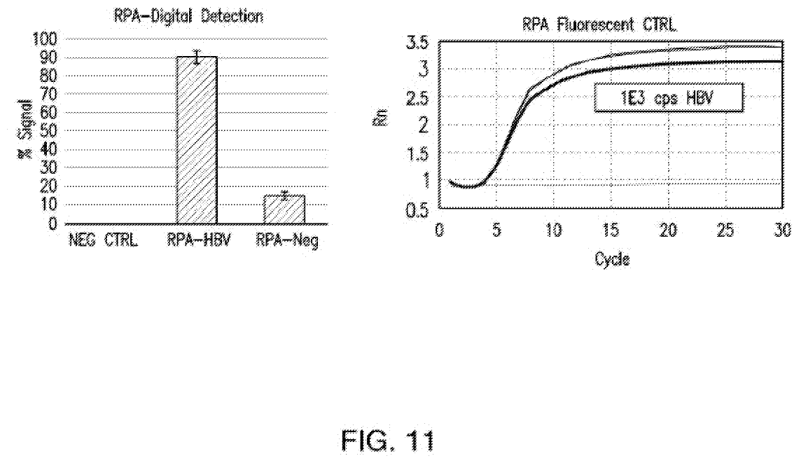

FIG. 11 illustrates exemplary results associated with RPA implementations of the disclosed subject matter in combination with a digital detection strategy. HBV DNA was added at 1×10³ IU/rxn to an RPA reaction using the following primers and probes. Rather than using the ExoIII enzyme for fluorescence detection, the Nfo exonuclease was added. The reverse primer contains a digoxigenin (DIG) label at the 5′ end and the probe contains a Biotin tag at the 5′ end. Upon amplification, the amplicon will contain a Biotin and DIG label that is then recognized by anti-DIG monoclonal antibody bound to microparticles. This complex is then combined with a conjugate (Streptavidin linked to alkaline phosphatase) and detection substrate, loaded into nanowells, and imaged. There graph at the top left demonstrates the percentage of positive wells (RPA-HBV) compared to the control (RPA-Neg) where no HBV DNA was added to the RPA reaction. A control reaction was generated using conventional fluorescent probe detection (top right). FIG. 13 compares an exemplary digital detection strategy as compared to an exemplary fluorescence detection strategy, e.g., a fluorescence detection strategy as described in Aspect 6, below.

### Example 6: NEAR-Based Amplification Implementation

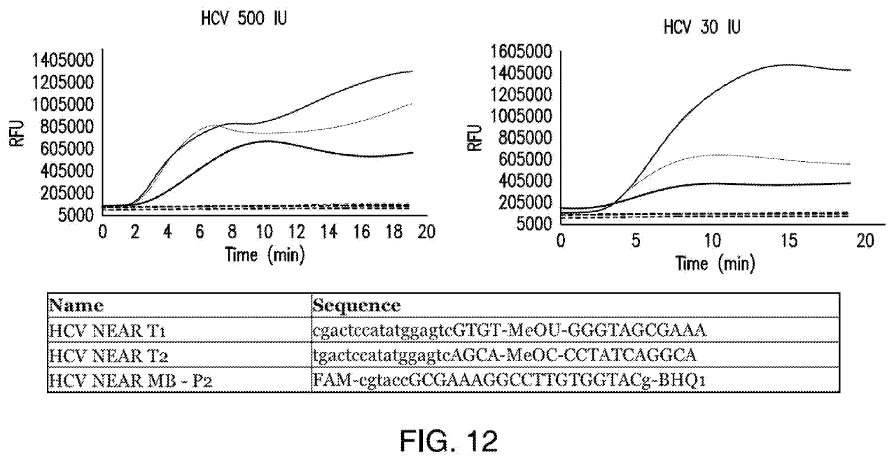

FIG. 12 illustrates exemplary results associated with NEAR implementations of the disclosed subject matter. HCV RNA was amplified by NEAR using the oligonucleotides listed. Fluorescence was measured over time (minutes) on a thermocycler incubated at 56° C. A positive signal is observed (solid lines) compared to water controls (dotted lines) at both 500 and 30 IU/reaction.

### Example 7: RPA-Based Amplification Implementation with Fluorescent Detection

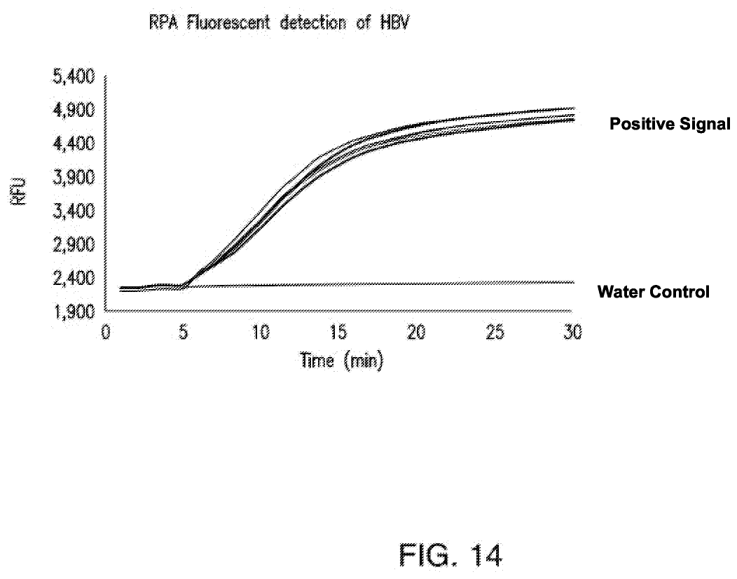

FIG. 14 illustrates exemplary results associated with RPA implementations of the disclosed subject matter in combination with a fluorescent detection strategy. HBV DNA was amplified by RPA using the oligonucleotides listed. Fluorescence was measured over time (minutes) on a thermocycler incubated at 40° C. A positive signal is observed (purple lines) compared to water controls (gray lines) at 5 IU/reaction.

### Example 8: Human Parvovirus B19 and Hepatitis A Virus

Human Parvovirus B19 (B19) and hepatitis A virus (HAV) are both non-enveloped, icosahedral viruses. The B19 genome is linear ssDNA (+/−), measures 5.6 kb in size, and encodes two open reading frames (ORFs): NS1 and VP1. Four B19 subtypes have been identified with GT1 comprising majority of clinical cases. The HAV genome consists of linear ssRNA (+) 7.5 kb in length. HAV encodes a single polypeptide preceded by a 5′ untranslated region (UTR). This 5′ UTR region is largely conserved amongst the six identified HAV strains.

Both B19 and HAV cause disease in humans, particularly in at risk populations such as pregnant women and immunocompromised individuals. Both viruses are resistant to microbial inactivation steps (e.g., SDS treatment, pasteurization) used in manufacturing of plasma-derived products. Thus, contamination of source plasma donations with either agent poses a transmission risk to recipients of products derived from human plasma.

For sample preparation, 1 mL of plasma or serum is added to 0.75 mL of Lysis buffer (3.13 M GITC, 6.7% Tween-20, 100 mM Tris, pH 7.8) together with Copper Titanium (CuTi) microparticles. In some instances, Proteinase K is also added. The solution is then incubated for 5 minutes with rotation to lyse the sample and bind the released nucleic acid to the solid phase. Microparticles are then transferred by a magnet to a well containing 500 ul of lysis buffer (Wash 1) and mixed for 1 minute. Particles are then captured and moved to another well containing 250 ul of water and then another well of 100 ul water (Wash 2 and 3). Next, bound nucleic acid is eluted off the microparticles by incubating in a 5 mM phosphate solution for 3 minutes at 80° C.

Amplification of human Parvovirus B19 and hepatitis A virus nucleic acid sequences was performed using Recombinase-Polymerase Amplification (RPA) with various combinations of amplification oligonucleotides and probe oligonucleotides. The combinations tested are shown Table 12.

TABLE 12
Combination of amplification and probe oligonucleotides for RPA

| | | Amplifi- | Amplifi- | |
|---|---|---|---|---|
| Combination | | cation | cation | |
| # | Target | Oligo #1 | Oligo #2 | Probe |
| 1 | Parvovirus B19 | VP1-FP5 | VP1-RP4 | VP1-P3 |
| 2 | Parvovirus B19 | VP1-FP5 | VP1-RP1 | VP1-P2 |
| 3 | Parvovirus B19 | VP1-FP5 | VP1-RP1 | VP1-P3 |
| 4 | Parvovirus B19 | VP1-FP5 | VP1-RP4 | VP1-P7 |
| 5 | Parvovirus B19 | NS1-FP6 | NS1-RP4 | NS1-P5 |
| 6 | Parvovirus B19 | NS1-FP6 | NS1-RP6 | NS1-P5 |
| 7 | Parvovirus B19 | NS1-FP3 | NS1-RP6 | NS1-P5 |
| 8 | Parvovirus B19 | NS1-FP3 | NS1-RP6 | NS1-P3 |
| 9 | Hepatitis A Virus | HAV-FP3 | HAV-RP2 | HAV-P1 |
| 10 | Hepatitis A Virus | HAV-FP6 | HAV-RP1 | HAV-P1 |
| 11 | Hepatitis A Virus | HAV-FP6 | HAV-RP4 | HAV-P1 |

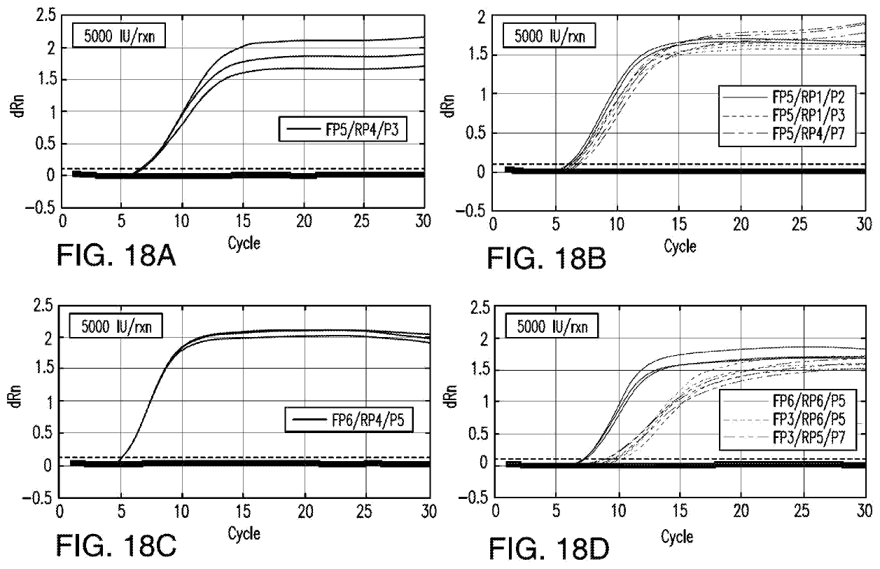

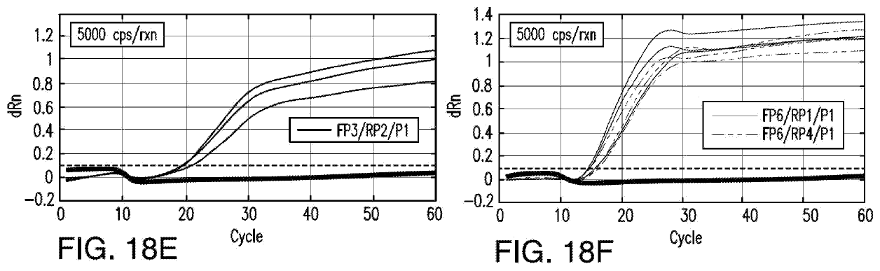

FIGS. 18A-18D show graphs of the dRn value over cycle number for two different combinations for each of two open reading frames (ORFs): NS1 and VP1 of parvovirus. FIG. 18A is combination 1, FIG. 18B shows combinations 2-4, FIG. 18C shows combination 5, and FIG. 18D shows combinations 6-8, as described in Table 5. The parvo target was amplified under the reaction conditions for all of the combinations, with the detectable level of amplification occurring within 10 cycles. The amplicon for the combinations 1 and 4 comprises a nucleic acid sequence at least 70% similar to that of TATCTGACCACCCCCATGCCTTATCATCCAGTAGC AGTCATGCAGAACCTAGAGGAGAAGATGCAGTATTATCTAGTGAAGACTTAC ACAAGCCTGGGCAAGTTAGCGTACAACTACCCGGTACTAACTATGTTGGGCCT GGCAATGAGCTACAAGCTGGGCCCCCGCAAAGTGCTGTTGACAGTGCTGCAA GGATTCATGACTTTAGGTATAGCCA. The amplicon for combination 5 comprises a nucleic acid sequence at least 70% similar to that of AGTGGTGGTGAAAGCTCTGAAGAACTCAGTGAAAGCAGCT TTTTTAACCTCATCACCCCAGGCGCCTGGAACACTGAAACCCCGCGCTCTAGT ACGCCCATCCCCGGGACCAGTTCAGGAGAATCATTTGTCGGAAGCCCAGTTTC CTC. Amplicons for alternative combinations of Parvovirus B19 targets comprise a nucleic acid sequence at least 70% similar to that of ATTTTCAAAGTCATGGACAGT TATCTGACCACCCCCATGCCTTATCATCCAGTAGCAGTCATGCAGAACCTAGA GGAGAAGATGCAGTATTATCTAGTGAAGACTTACACAAGCCTGGGCAAGTTA GCGTACAACTACCCGGTACTAACTATGTTGGGCCTGGCAATGAGCTACAAGCT GGGCCCCCGCAAAGTGCTGTTGACAGTGCTGCAAGGATTCATGACTTTAGGTA TAGC or AGTATCAGC AGCAGTGGTGGTGAAAGCTCTGAAGAACTCAGTGAAAGCAGCTTTTTTAACCT CATCACCCCAGGCGCCTGGAACACTGAAACCCCGCGCTCTAGTACGCCCATCC CCGGGACCAGTTCAGGAGAATCATTTGTCGGAAGCCCAGTTTCCTCCGAAGTT GTAGC.

FIGS. 18E and 18F show graphs of the dRn value over time for two different combinations targeting the 5′ untranslated region (UTR) of the single HAV polypeptide. FIG. 18E is combination 9 and FIG. 18F shows combinations 10-11, as described in Table 5. The HAV target was amplified under the reaction conditions for all of the combinations, most of which reached detectable levels within 20 cycles The amplicon for combination 9 comprises a nucleic acid sequence at least 70% similar to that of TCCATGATTAGCATGGAGCTGTAGGAGTCTAAATTGGGGACGCAGATGTTTGG GACGTCACCTTGCAGTGTTAACTTGGCTTTCATGAACCTCTTTGATCTTTCACA AGAGGTAGGCTACGGGTGAAACCTCTTAGGCTAATACTTCTATGAAGAGATGC CTTGGATAGGGTAACAGCGGCG. Amplicons for alternative combinations of Parvovirus B19 targets comprise a nucleic acid sequence at least 70% similar to that of TCCATGATTAGCATGGAGCTGTAGGAGTCTAAATTGGGGACGCAGATGTTTGG GACGTCACCTTGCAGTGTTAACTTGGCTTTCATGAACCTCTTTGATCTTTCACA AGAGGTAGGCTACGGGTGAAACCTCTTAGGCTAATACTTCTATGAAGAGATGC CTTGGATAGGGTAACAGCGGCGGATATTGGTGAGT.

### Example 9: Babesia Parasite

Babesia parasite is transmitted by ticks into human causing infection of red blood cells, known as babesiosis. Babesia is the most common transfusion-transmitted parasite in the United States, primarily caused by the four main Babesia species (B. microti, B. duncani, B. divergens, B. venatorum) in human whole blood.

Whole blood samples can be processed by adding 150 ul of sample to 850 ul of lysis buffer in a pre-treatment step of mixing sample to lysis buffer in a 1:1 ratio. Following pre-treatment, the lysate then follows the same process as in Example 8, above. Amplification of Babesia parasite nucleic acid sequences was performed using Recombinase-Polymerase Amplification (RPA) with various combinations of amplification oligonucleotides and probe oligonucleotides. The Babesia target nucleic acid is inside the 18S ribosomal RNA gene. The combinations tested are shown Table 13. Each combination resulted in a positive signal.

TABLE 13
Combination of amplification and probe oligonucleotides for RPA

| Combination | Amplification | Amplification | |
|---|---|---|---|
| No: | Oligo #1 | Oligo #2 | Probe |
| 1 | 18S FP4 | 18S RP13 | 18S P2 |
| 2 | 18S FP6 | 18S RP13 | 18S P2 |
| 3 | 18S FP6 | 18S RP8 | 18S P2 |
| 4 | 18S FP6 | 18S RP7 | 18S P2 |

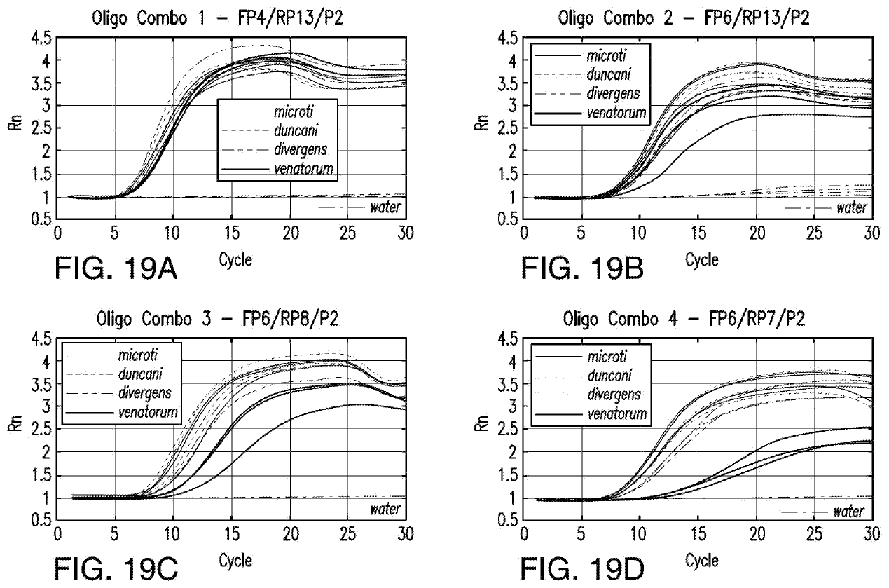

FIGS. 19A-19D show graphs of the dRn value over cycle number for each of the combinations. The Babesia parasite nucleic acid target was amplified for all four main Babesia species under the reaction conditions for all of the combinations.

### Example 10: HxV

Sample preparation was performed as outlined in Example 8, above. Amplification of multiple HxV viral target (human immunodeficiency virus 1 (HIV-1), human immunodeficiency virus 2 (HIV-2), hepatitis B virus (HBV), and hepatitis C virus (HCV)) nucleic acid sequences was performed using Recombinase-Polymerase Amplification (RPA) with various combinations of amplification oligonucleotides and probe oligonucleotides. The combinations tested are shown Table 14.

In addition, the assay used an internal control that amplifies a region within the hydroxypyruvate reductase gene (HPR) from the pumpkin plant that is unrelated to the HIV-1, HIV-2, HBV, and HCV sequences. The internal control RNA was encapsulated within a bacteriophage protein along with coliphage MS2 packaging sequences (armored RNA) and amplified oligonucleotides comprising TAATGAAGTAGGACTTTGGTTCTATTTTGTTGGT TATTGAGCCAGAGTAATGGTTAATAGGAGCAGTTGGGGGCATTCGTATTTAAC TGTCAGAGGTGAAATTCTTAGATTTGTTAAAGACGAACTACTGCGAAAGCATT TGCCAAGGA and TAATGAAGTAGGACTTTGGTTCTATTTTGTTGGTT ATTGAGCCAGAGTAAT GGTTAATAGGAGCAGTTGGGGGCATTCGTATTTAACTGTCAGAGGTGAAATTC TTAGATTTGTTAAAGACGAACTACTGCGAAAGCATTTGCCAAGGATGTTTTCA TTAATCAAGAACGAAAGT, with a probe oligonucleotide sequence comprising TAATGAAGTAG GACTTTGGTTCTATTTTGTTGGTTATTGAGCCAGAGTAATGGTTAATAGGAGCA GTTGGGGGCATTCGTATTTAACTGTCAGAGGTGAAATTCTTAGATTTGTTAAA GACGAACTACTGCGAAAGCATTTGCCAAGGATGTTTTCATTAATCAAGAACGA AAGTTAGGGG.

TABLE 14
Combination of amplification and probe oligonucleotides for RPA

| | | Amplification | Amplification | |
|---|---|---|---|---|
| Target | Region | Oligo #1 | Oligo #2 | Probe |
| HIV-1 | INT | HIV-1 INT FP1 | HIV-1 INT RP7 | HIV-1 INT P4 |
| HIV-1 | LTR | HIV-1 LTR FP1 | HIV-1 LTR RP3 | HIV-1 LTR P1 |
| HCV | 5′ UTR | HCV FP6a | HCV RP2a | HCV RC3 |
| HBV | HBs | HBV-FP1a-M5 | HBV-RP | HBV-P5 |
| HIV-2 | Pol | HIV-2 POL FP2 | HIV-2 POL RP8 | HIV-2 POL P2 |

Inclusivity was demonstrated by analyzing the sequence of each of the primers and probes for homology with various Genotypes or Groups of HxV viral sequences (Table 15).

TABLE 15
HxV RPA Inclusivity Screening:

| | | Genotypes or | # Samples | # Samples |
|---|---|---|---|---|
| Target | Region | Groups | Tested | Detected |
| HIV-1 | INT/LTR | M, O, N | 19 | 19 |
| HCV | 5′ UTR | 1, 2, 3, 4, 5, 6 | 11 | 11 |
| HBV | HBs | A, B, C, D, E, F, G, H | 22 | 22 |
| HIV-2 | Pol | A, B | 60 | 55 |

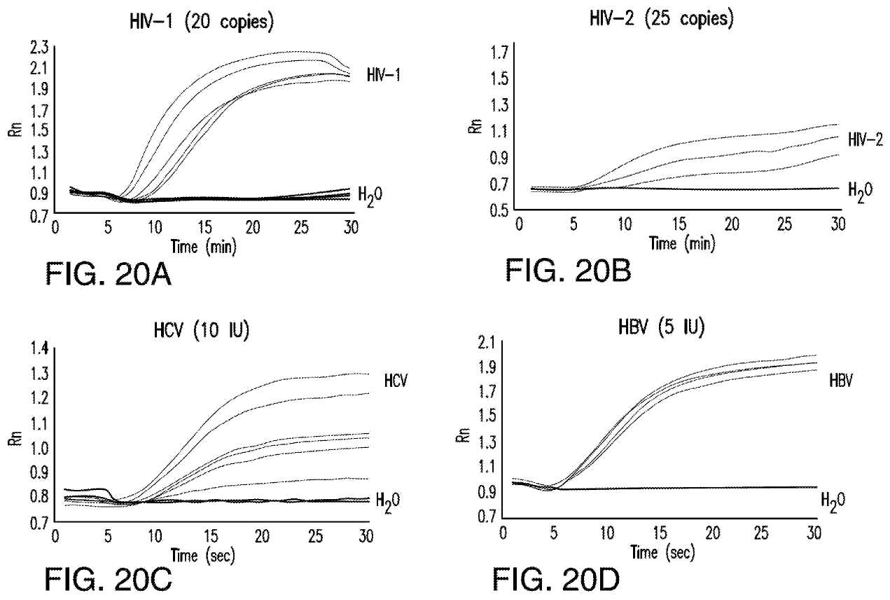

FIGS. 20A-20D show graphs of the dRn value over time for each of the combinations. Amplification was seen for each of HIV-1 (FIG. 20A), HIV-2 (FIG. 20B), HCV (FIG. 20C), and HBV (FIG. 20D) under the reaction conditions, with the detectable level of amplification occurring within approximately 15 minutes in most cases Example 11: Reverse Transcriptase Evaluation

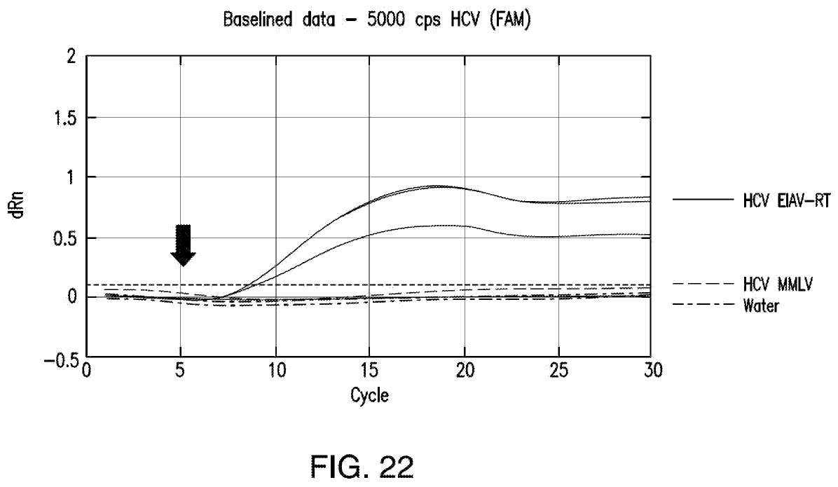

FIG. 22 depicts the results obtained in RPA-based analysis of HCV in the presence of EIAV-RT. Sample preparation was performed as outlined in Example 8, above. Amplification of nucleic acid sequences was performed using RPA with various enzymes in accordance with the protocol illustrated in FIG. 21. The following Reverse Transcriptases (RT) were compared in using the method of FIG. 21: OmniScript (Qiagen), SensiScript (Qiagen), MonsterScript (Epicentre), Transcriptor (Roche), HIV RT (Ambion), Superscript III (Invitrogen), ThermoScript (Invitrogen), Thermo-X (Invitrogen), ImProm II (Promega) (data not shown). As illustrated in FIG. 22, EIAV-RT provided optimum activity with HCV. EIAV-RT also provided improvement to HIV-1 detection with no impact to HBV detection (data not shown).

### Example 12: Improvement of HCV/HBV Multiplex Analysis

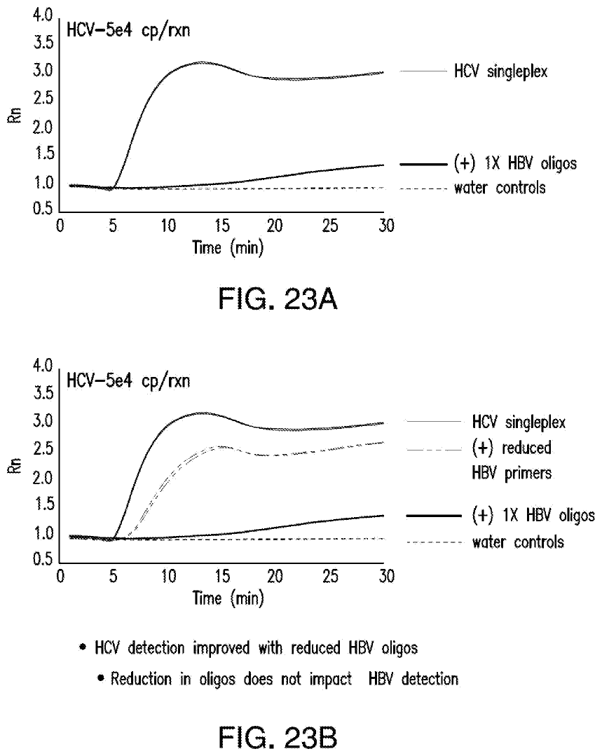

FIGS. 23A-23B depict the results obtained in HCV/HBV multiplex analysis in the presence of differing concentrations of HBV oligonucleotides (i.e., primers and probes). Sample preparation was performed as outlined in Example 8, above. Amplification of HCV and HBV nucleic acid sequences was performed using Recombinase-Polymerase Amplification (RPA) with various combinations of amplification oligonucleotides and probe oligonucleotides in accordance with the protocol illustrated in FIG. 21. As indicated in FIG. 23B, HCV detection was improved in the presence of a reduced concentration of HBV oligonucleotides. The reduction in HBV oligonucleotides did not impact HBV detection (data not shown).

### Example 13: HxV Singleplex and Multiplex Analysis

The reagent concentrations associated with both conventional singleplex RPA-based analysis (“Singleplex Concentrations”) and reagent concentrations associated with the HxV multiplex analyses of the instant example are identified in Table 16. Sample preparation prior to amplification was performed as outlined in Example 8, above.

TABLE 16

| | Singleplex | | HxV | |
|---|---|---|---|---|
| Reagent | Concentration | | Concentrations | |
| Nuclease-free water | N/A | | N/A | |
| Tris-HCl 1M, pH 8.3 | 50 | mM | 50 | mM |
| DTT | 5 | mM | 5 | mM |
| Potassium Acetate | 100 | mM | 100 | mM |
| 20% Polyethylene glycol | 5.5% | | 5.5% | |
| 100 mM dNTPs | 1.8 | mM | 2.7 | mM |
| ATP | 2.5 | mM | 3.5 | mM |
| Phosphocreatine | 50 | mM | 50 | mM |
| Forward Primer | 420 | nM | 157.50/39.37/86.13* | nM |
| Reverse Primer | 420 | nM | 236.37/39.37//86.13* | nM |
| Exo Probe | 120 | nM | 90/22.5/90* | nM |
| ROX reference dye | 15 | nM | 45 | nM |
| Glycerol | 6.5% | | 8.4% | |
| Creatine Kinase | 0.1 | mg/mL | 0.1 | mg/mL |
| UvsX | 0.3 | mg/mL | 0.375 | mg/mL |
| UvsY | 0.09 | mg/mL | 0.0675 | mg/mL |
| DNA Polymerase | 0.0798 | mg/mL | 0.1396 | mg/mL |
| Gp32 | 0.48 | mg/mL | 1.200 | mg/mL |
| Exonuclease III | 0.1 | mg/mL | 0.1 | mg/mL |
| Bovine Serum Albumin (BSA) | 0.02 | mg/mL | 0.02 | mg/mL |
| EIAV Reverse Transcriptase | 0.0008 | mg/mL | 0.0016 | mg/mL |
| Magnesium Acetate | 14 | mM | 14 | mM |
| Nuclease-free water | N/A | | N/A | |
| Tris-HCl 1M, pH 8.3 | 50 | mM | 50 | mM |
| DTT | 5 | mM | 5 | mM |
| Potassium Acetate | 100 | mM | 100 | mM |
| 20% Polyethylene glycol | 5.5% | | 5.5% | |
| 100 mM dNTPs | 1.8 | mM | 2.7 | mM |
| ATP | 2.5 | mM | 3.5 | mM |
| Phosphocreatine | 50 | mM | 50 | mM |
| Forward Primer | 420 | nM | 157.50/86.13* | nM |
| Reverse Primer | 420 | nM | 236.37/86.13* | nM |
| Exo Probe | 120 | nM | 90/90* | nM |
| ROX reference dye | 15 | nM | 45 | nM |
| Glycerol | 6.5% | | 8.4% | |
| Creatine Kinase | 0.1 | mg/mL | 0.1 | mg/mL |
| UvsX | 0.3 | mg/mL | 0.375 | mg/mL |
| UvsY | 0.09 | mg/mL | 0.0675 | mg/mL |
| DNA Polymerase | 0.0798 | mg/mL | 0.1396 | mg/mL |
| Gp32 | 0.48 | mg/mL | 1.200 | mg/mL |
| Exonuclease III | 0.1 | mg/mL | 0.1 | mg/mL |
| Bovine Serum Albumin (BSA) | 0.02 | mg/mL | 0.02 | mg/mL |
| EIAV Reverse Transcriptase | 0.0008 | mg/mL | 0.0016 | mg/mL |
| Magnesium Acetate | 14 | mM | 14 | mM |

*[HIV-1 INT Oligos/HIV-1 LTR Oligos/HBV Oligos]
*[HIV-1 Oligos/HBV Oligos]

FIG. 24 depicts the results obtained in HIV-1 singleplex analysis. Amplification of HIV-1 nucleic acid was performed using RPA with the identified primer oligonucleotides and probe oligonucleotides in accordance with the protocol illustrated in FIG. 21 and the singleplex reagent concentrations (“Singleplex Concentrations”) of Table 16.

FIG. 25 depicts the results obtained in HIV-2 singleplex analysis. Amplification of HIV-2 nucleic acid was performed using RPA with the identified primer oligonucleotides and probe oligonucleotides in accordance with the protocol illustrated in FIG. 21 and the singleplex reagent concentrations (“Singleplex Concentrations”) of Table 16.

FIG. 26 depicts the results obtained in HBV singleplex analysis. Amplification of HBV nucleic acid was performed using RPA with the identified primer oligonucleotides and probe oligonucleotides in accordance with the protocol illustrated in FIG. 21 and the singleplex reagent concentrations (“Singleplex Concentrations”) of Table 16.

FIG. 27 depicts the results obtained in HCV singleplex analysis. Amplification of HCV nucleic acid was performed using RPA with the identified primer oligonucleotides and probe oligonucleotides in accordance with the protocol illustrated in FIG. 21 and the singleplex reagent concentrations (“Singleplex Concentrations”) of Table 16.

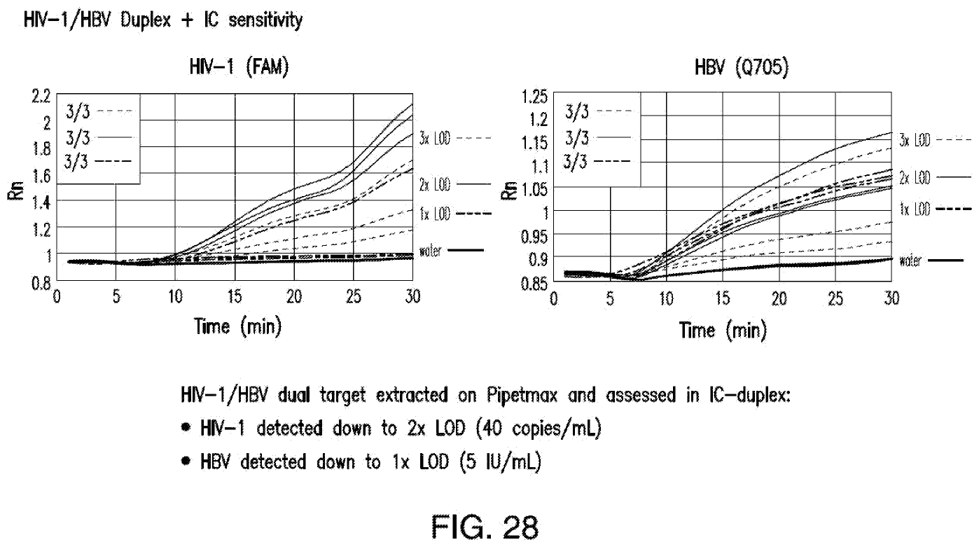

FIG. 28 depicts the results obtained in HIV-1/HBV+Internal Control multiplex analysis. Amplification of HIV-1/HBV+Internal Control nucleic acid was performed using RPA with the primer oligonucleotides and probe oligonucleotides identified herein (FIG. 26—HIV-1; FIG. 28—HBV; and Example 10—Internal Control) in accordance with the protocol illustrated in FIG. 21 and the multiplex reagent concentrations of Table 16.

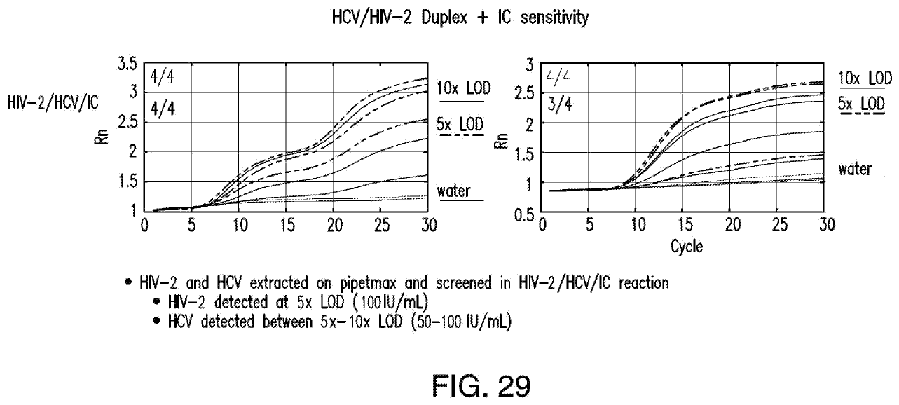

FIG. 29 is a graph that depicts the results obtained in HIV-2/HCV+Internal Control multiplex analysis. Amplification of HIV-2/HCV+Internal Control nucleic acid was performed using RPA with the primer oligonucleotides and probe oligonucleotides identified herein (FIG. 25—HIV-2; FIG. 27—HCV; and Example 10—Internal Control) in accordance with the protocol illustrated in FIG. 21 and the multiplex reagent concentrations of Table 16.

### Example 14: HAV & Parvovirus B19 Multiplex Analysis

FIG. 30A-30B depicts the results obtained in HAV/Parvovirus B19 multiplex analysis. Whole blood samples can be processed by adding 150 ul of sample to 850 ul of lysis buffer in a pre-treatment step of mixing sample to lysis buffer in a 1:1 ratio. Following pre-treatment, the lysate then follows the same process as in Example 8, above. Amplification of HAV/Parvovirus B19 nucleic acid was performed using RPA with the identified primer oligonucleotides and probe oligonucleotides in accordance with the protocol illustrated in FIG. 21 and the multiplex reagent concentrations of Table 17.

### Example 15: Quantitative Parvovirus B19 Analysis

In order to produce quantitative amplification of Parvovirus B19, serial dilutions of positive plasma samples were prepared and subject to sample extraction to isolate the nucleic acid. Following sample extraction, as outlined in Example 14, RPA reactions were set up as described herein, see e.g., FIG. 21, using the reagent concentrations identified in Table 17.

TABLE 17

| | Singleplex | | Parvo/HAV | |
|---|---|---|---|---|
| Reagent | Concentration | | Concentration | |
| Nuclease-free water | N/A | | N/A | |
| Tris-HCl 1M, pH 8.3 | 50 | mM | 50 | mM |
| DTT | 5 | mM | 5 | mM |
| Potassium Acetate | 100 | mM | 100 | mM |
| 20% Poly(ethylene glycol | 5.5% | | 5.5% | |
| 100 mM dNTPs | 1.8 | mM | 1.8 | mM |
| ATP | 2.5 | mM | 3.5 | mM |
| Phosphocreatine | 50 | mM | 50 | mM |
| Forward Primer | 420 | nM | 147.5/393.8* | nM |
| Reverse Primer | 420 | nM | 147.5/236.3* | nM |
| Exo Probe | 120 | nM | 85/120* | nM |
| ROX reference dye | 15 | nM | 15 | nM |
| Glycerol | 6.5% | | 8.4% | |
| Creatine Kinase | 0.1 | mg/mL | 0.035 | mg/mL |
| UvsX | 0.3 | mg/mL | 0.42 | mg/mL |
| UvsY | 0.09 | mg/mL | 0.126 | mg/mL |
| DNA Polymerase | 0.0798 | mg/mL | 0.168 | mg/mL |
| Gp32 | 0.48 | mg/mL | 0.672 | mg/mL |
| Exonuclease III | 0.1 | mg/mL | 0.1 | mg/mL |
| Bovine Serum Albumin | 0.02 | mg/mL | 0.02 | mg/mL |
| (BSA) | | | | |
| EIAV Reverse Transcriptase | 0.0008 | mg/mL | 0.0016 | mg/mL |
| Magnesium Acetate | 14 | mM | 14 | mM |

*[Parvo Oligos/HAV Oligos]

This testing determined if the RPA reaction demonstrated linearity and formulation changes were made (e.g. enzyme concentrations, see Table 17) to allow for greater separation between target levels and improved linearity.

For quantitation, the time at which the amplification signal crossed a designated threshold (Cycle Threshold, Ct) is measured during the reaction. This was then compared to the signal of samples at known concentrations (Calibrators). The concentration of the unknown sample can then be determined when compared to the Ct of the calibrators. FIG. 31A is a graph of the singleplex RPA of Parvovirus B19 at distinct target nucleic acid concentrations. FIG. 31B and FIG. 31C are graphs indicating the relationship of the results indicating linearity of assay as well as specific data underlying the graphs (FIG. 31D).

### Example 16: Chikungunya Virus RPA Analysis

The Chikungunya Virus genome aligned with the primers and probes described herein is depicted in FIG. 32A, with the sequences of the primer and probe oligonucleotides presented in FIG. 32D. Graphs of the singleplex RPA of Chikungunya Virus using the indicated amplification and probe oligonucleotides are provided in FIG. 32B and FIG. 32C. Sample preparation was performed as outlined in Example 8, above. Amplification of Chikungunya Virus nucleic acid was performed using RPA with the identified primer oligonucleotides and probe oligonucleotides in accordance with the protocol illustrated in FIG. 21 and the singleplex reagent concentrations (“Singleplex Concentrations”) of Table 16.

### Example 17: Dengue Virus RPA Analysis

FIGS. 33A-33C include a graph of the singleplex RPA of Dengue Virus using the indicated conditions (FIG. 33B); as well as the sequences of the indicated primers and probe (FIG. 33C). Sample preparation was performed as outlined in Example 8, above. Amplification of Dengue Virus nucleic acid was performed using RPA with the identified primer oligonucleotides and probe oligonucleotides in accordance with the protocol illustrated in FIG. 21 and the singleplex reagent concentrations (“Singleplex Concentrations”) of Table 16, except as noted in FIG. 33B.

### Example 18: West Nile Virus RPA Analysis

FIG. 34A depicts the location of the primers and probe relative the West Nile Virus (WNV) genome and the sequences of the indicated primer and probe oligonucleotides are presented in FIG. 34D. Graphs of the singleplex RPA of WNV using the indicated amplification and probe oligonucleotides are presented in FIG. 34B and FIG. 34C. A graph of the singleplex RPA of WNV using the indicated amplification and probe oligonucleotides at different concentrations of target nucleic acid is presented in FIG. 34E. Sample preparation was performed as outlined in Example 8, above. Amplification of West Nile Virus nucleic acid was performed using RPA with the identified primer oligonucleotides and probe oligonucleotides in accordance with the protocol illustrated in FIG. 21 and the singleplex reagent concentrations (“Singleplex Concentrations”) of Table 16.

### Example 19: Zika Virus RPA Analysis

FIG. 35A depicts the location of the primer and probe oligonucleotides relative the Zika Virus genome and the sequences of the indicated primers and probe are presented in FIG. 35C. A graph of the singleplex RPA of Zika Virus using the indicated amplification and probe oligonucleotides is presented in FIG. 35B. Sample preparation was performed as outlined in Example 8, above. Amplification of Zika Virus nucleic acid was performed using RPA with the identified primer oligonucleotides and probe oligonucleotides in accordance with the protocol illustrated in FIG. 21 and the singleplex reagent concentrations (“Singleplex Concentrations”) of Table 16.

### Example 20: Babesia RPA Analysis

FIGS. 36A-36B are graphs of the multiplex RPA of Babesia (FIG. 36A) and an internal control (FIG. 36B) using the indicated Babesia amplification and probe oligonucleotides (FIG. 36C) and the Internal Control nucleic acids describe in Example 10. Sample preparation was performed as outlined in Example 14, above. Amplification of Babesia and Internal Control nucleic acid was performed using RPA with the identified primer oligonucleotides and probe oligonucleotides in accordance with the protocol illustrated in FIG. 21 and the multiplex reagent concentrations of Table 16.

### Example 21: Malaria RPA Analysis

FIGS. 37A-37B include graphs of the multiplex RPA of Malaria (FIG. 37A) and an internal control (FIG. 37B) using the indicated Malaria amplification and probe oligonucleotides (FIG. 37C) and the Internal Control nucleic acids describe in Example 10. Sample preparation was performed as outlined in Example 14, above. Amplification of Malaria and Internal Control nucleic acid was performed using RPA with the identified primer oligonucleotides and probe oligonucleotides in accordance with the protocol illustrated in FIG. 21 and the multiplex reagent concentrations of Table 16.

### Example 22: SARS-CoV-2 (COVID-19) RPA Analysis

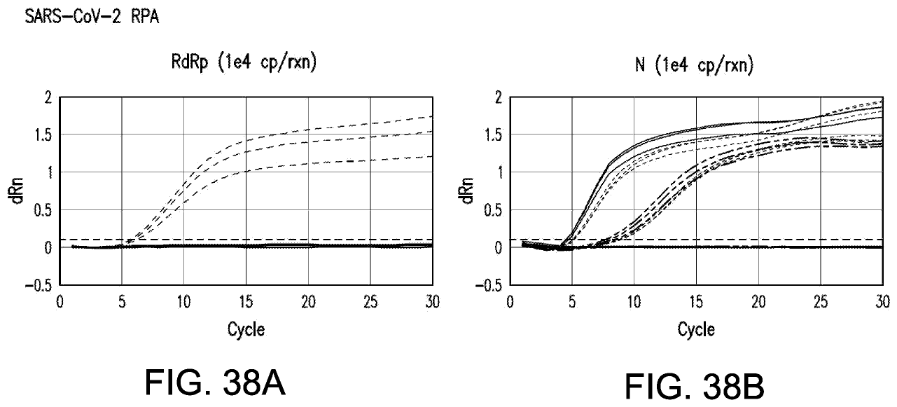

FIGS. 38A-38B include graphs of the RPA analysis of SARS-CoV-2 (COVID-19) directed to a first target sequence (Combination 1) FIG. 38A or a second target sequence (Combinations 2-5) FIG. 38B. Sample preparation was performed as outlined in Example 8, above. Amplification SARS-CoV-2 (COVID-19) nucleic acid was performed using RPA with the combinations of primer oligonucleotides and probe oligonucleotides as follows (sequences provided above) and in accordance with the protocol illustrated in FIG. 21 and the multiplex reagent concentrations of Table 16.

| Combina- | | | | Positive |
|---|---|---|---|---|
| tion No: | FP | RP | Probe | Signal |
| 1 | COVID-19 FP1 | COVID-19 RP1 | COVID-19 P1 | Y |
| 2 | COVID-19 FP2 | COVID-19 RP2 | COVID-19 P2 | Y |
| 3 | COVID-19 FP2 | COVID-19 RP2 | COVID-19 P3 | Y |
| 4 | COVID-19 FP3 | COVID-19 RP2 | COVID-19 P4 | Y |
| 5 | COVID-19 FP3 | COVID-19 RP3 | COVID-19 P4 | Y |

### Example 23: Time to Result (TTR) for Pathogen or Infectious Agent Detection

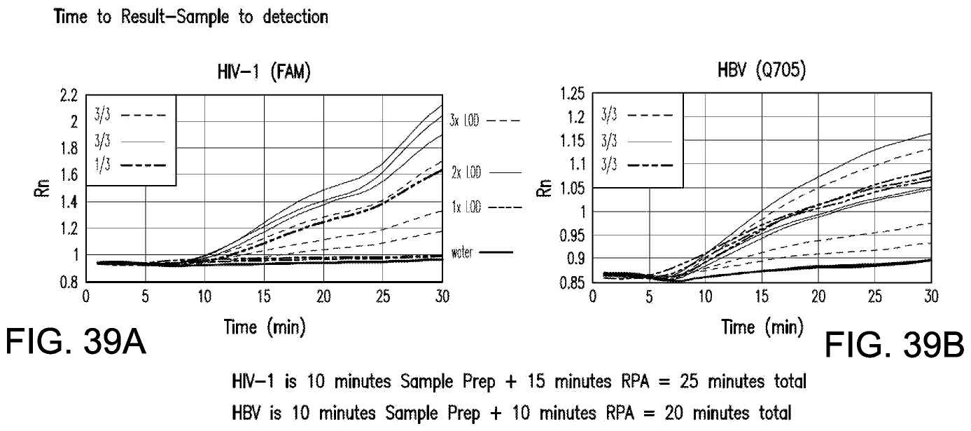

FIGS. 39A-39B include graphs of the multiplex RPA of HIV-1 (FIG. 39A) and HBV (FIG. 39B) indicating that the indicated limits of detection can be achieved using the methods of the present disclosure in the time frames indicated. Sample preparation was performed as outlined in Example 8, above. Amplification of HIV-1 and HBV nucleic acid was performed using RPA with the primer oligonucleotides and probe oligonucleotides identified herein (FIG. 24—HIV-1 and FIG. 26—HBV) in accordance with the protocol illustrated in FIG. 21 and the multiplex reagent concentrations of Table 16.

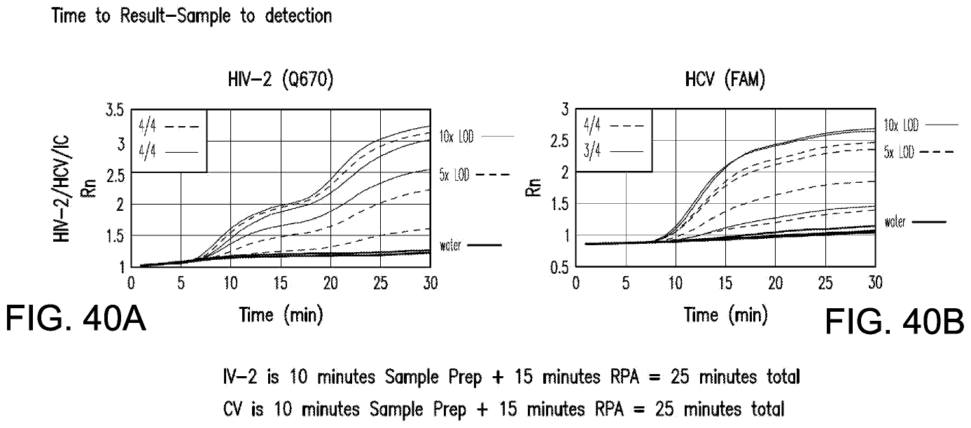

FIGS. 40A-40B include graphs of the multiplex RPA of HIV-2 (FIG. 40A) and HCV (FIG. 40B) indicating that the indicated limits of detection can be achieved using the methods of the present disclosure in the time frames indicated. Amplification of HIV-2 and HCV nucleic acid was performed using RPA with the primer oligonucleotides and probe oligonucleotides identified herein (FIG. 25—HIV-2 and FIG. 27—HCV) in accordance with the protocol illustrated in FIG. 21 and the multiplex reagent concentrations of Table 16.

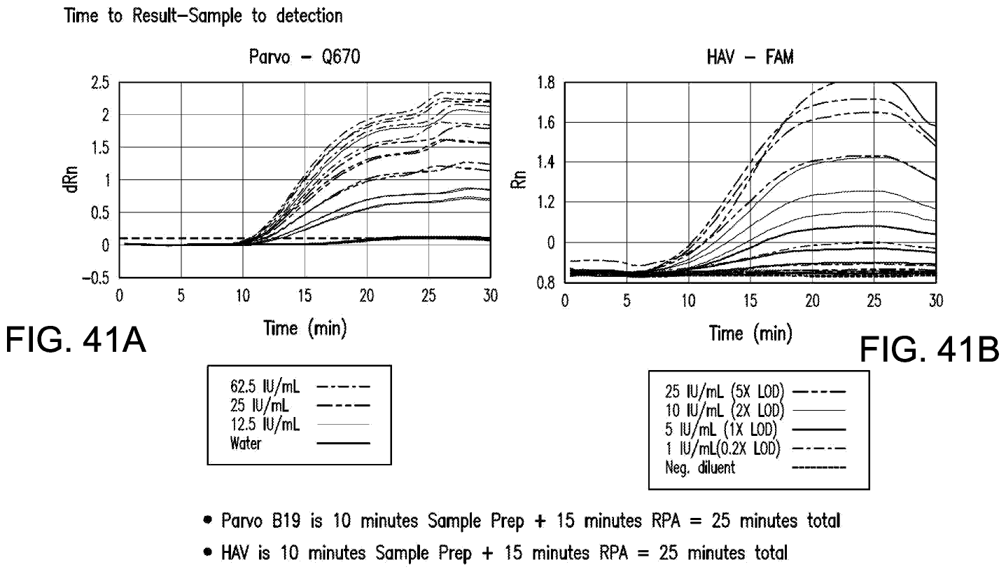

FIGS. 41A-41B include graphs of the multiplex RPA of Parvo (FIG. 41A) and HAV (FIG. 41B) indicating that the indicated limits of detection can be achieved using the methods of the present disclosure in the time frames indicated. Amplification of HAV and Parvovirus B19 nucleic acid was performed using RPA with the primer oligonucleotides and probe oligonucleotides identified herein (FIG. 30) in accordance with the protocol illustrated in FIG. 21 and the multiplex reagent concentrations of Table 16.

### Example 24: HxV Multiplex RPA

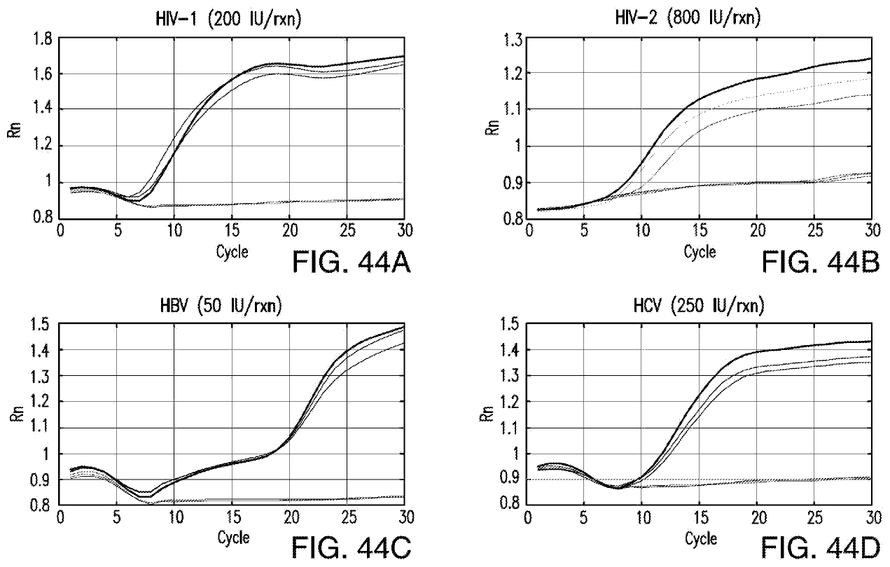

FIGS. 44A-44D include graphs of the multiplex RPA of HIV-1 (FIG. 44A), HIV-2 (FIG. 44B), HBV (FIG. 44C), and HCV (FIG. 44D) indicating that the indicated limits of detection can be achieved using the methods of the present disclosure. Sample preparation was performed as outlined in Example 8, above. Amplification of nucleic acid was performed using RPA with the primer oligonucleotides and probe oligonucleotides identified in Table 18.

TABLE 18

| Oligo Name | Concentration | |
|---|---|---|
| HIV-1 INT FP14 | 157.5 | nM |
| HIV-1 INT RP7 | 236.375 | nM |
| HIV-1 INT P4 | 45 | nM |
| HIV1 LTR FP1 | 39.375 | nM |
| HIV1 LTR RP3 | 39.375 | nM |
| HIV1 LTR Exo P1 | 22.5 | nM |
| HBV-FP1A-M5 | 86.135 | nM |
| HBV-RP | 86.135 | nM |
| HBV Probe P5 Q705 | 45 | nM |
| HCV FP6a | 420 | nM |
| HCV RP2a | 420 | nM |
| HCV probe RC3MOD-FAM | 90 | nM |
| HIV-2 POL FP3 | 78.75 | nM |
| HIV-2 POL RP6M | 90 | nM |
| IC FP6 | 142.5 | nM |
| IC RP2 | 142.5 | nM |
| IC P1 Cal560 | 67.5 | nM |

Amplification of nucleic acid was performed using RPA with the multiplex reagent concentrations of Table 19.

TABLE 19

| | Singleplex | | HxV | |
|---|---|---|---|---|
| Reagent | Concentration | | Concentrations | |
| Nuclease-free water | N/A | | N/A | |
| Tris-HCl 1M, pH 8.3 | 50 | mM | 50 | mM |
| DTT | 5 | mM | 5 | mM |
| Potassium Acetate | 100 | mM | 100 | mM |
| 20% Polyethylene glycol | 5.5% | | 5.5% | |
| 100 mM dNTPs | 1.8 | mM | 2.7 | mM |
| ATP | 2.5 | mM | 3.5 | mM |
| Phosphocreatine | 50 | mM | 50 | mM |
| ROX reference dye | 15 | nM | 45 | nM |
| Glycerol | 6.5% | | 8.4% | |
| Creatine Kinase | 0.1 | mg/mL | 0.1 | mg/mL |
| UvsX | 0.3 | mg/mL | 0.375 | mg/mL |
| UvsY | 0.09 | mg/mL | 0.0675 | mg/mL |
| DNA Polymerase | 0.0798 | mg/mL | 0.1396 | mg/mL |
| Gp32 | 0.48 | mg/mL | 1.200 | mg/mL |
| Exonuclease III | 0.1 | mg/mL | 0.1 | mg/mL |
| Bovine Serum Albumin (BSA) | 0.02 | mg/mL | 0.02 | mg/mL |
| EIAV Reverse Transcriptase | 0.0008 | mg/mL | 0.0016 | mg/mL |
| Magnesium Acetate | 14 | mM | 14 | mM |

### Example 25: Carousel Mixing

Mixing of the contents of a lysis tube was performed using successive oscillation of the sample transport, e.g., successive rotation of a rotatable sample preparation carousel. With reference to FIG. 64A, the sample transport can include a rotatable carousel. As embodied herein, the sample transport used had an inner radius R1 of 117 mm and an outer radius R2 of 133 mm. An electric motor was used to rotate the rotatable carousel. The electric motor used had a maximum motor velocity of 6,900 motor steps per second, a maximum motor acceleration of 34,500 motor steps per second², and 4,000 motor steps per revolution of the motor. The electric motor was coupled to the rotatable carousel using a gear ratio of 336:25, corresponding to 13.44 motor revolutions per carousel revolution.

Lysis tubes having a conical bottom and a 1 degree draft angle were loaded into the rotatable carousel. The configuration of the lysis tubes used in this example was similar to the configuration of exemplary lysis tube depicted in FIGS. 69C and 69D. The 1 degree draft angle of the lysis tubes used in this example was measured as the angle of the lysis tube side wall relative to a vertical axis of the lysis tube. The lysis tubes used in this example had a sidewall height of approximately 50 mm and a diameter of approximately 16 mm measured at the opening of the lysis tube. Additionally, the lysis tubes used in this example had a diameter of approximately 15 mm measured at the bottom of the lysis tube above the conical bottom. The lysis tubes used in this example had a conical bottom, with the conical bottom extending from the lysis tube sidewall at an angle of approximately 60 relative to a vertical axis of the lysis tube. 2 ml of liquid was placed in each lysis tube.

The carousel was oscillated successively by rotating the carousel successively clockwise and counterclockwise to mix the contents of the lysis tubes. For purpose of the example, rotation of the carousel was performed as follows: The carrousel was rotated 200 motor counts clockwise using a reciprocating period of 128 msec for the carousel to rotate the 200 motor counts. The carousel paused for a dwell time period of 5 msec, followed by a counterclockwise rotation of 200 motor counts using a reciprocating period of 128 msec. The pattern of 200 motor count clockwise rotation followed by 200 motor count counter-clockwise rotation was repeated for a period of approximately 5.2 seconds, after which time the contents of the lysis tube were found to be fully mixed.

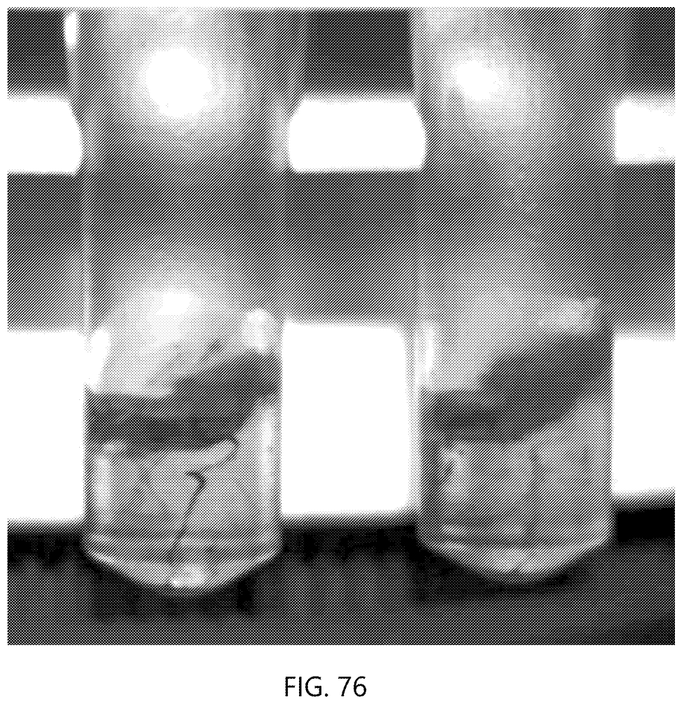

FIG. 76 depicts the contents of a lysis tube during the described successive clockwise and counterclockwise rotation of the carousel. As shown, rotation of the carousel caused the lysis tube to move in an arc, and caused a vortex to form in the liquid contents of the lysis tube. As described above, formation of the vortex within the lysis tube during successive clockwise and counter-clockwise rotation of the lysis tube can be a result of different acceleration forces at inner and outer wall portions of the lysis tube during movement of the lysis tube in an arc as a result of the rotation and acceleration of the carousel. In this example, a vortex was formed within the liquid contents of the lysis tube within approximately 1.3 seconds of successive clockwise and counterclockwise rotation as described above. In the example, during mixing of the contents of the lysis tube, the liquid contents of the lysis tube had a mixing height of approximately 24 mm above the bottom of the lysis tube. As described above, mixing the contents of the lysis tube using rotational movement of the carousel can be beneficial and can, among other things, reduce or eliminate the need for covering the lysis tube, such as with a disposable cover, during mixing. For example, and as shown in FIG. 76, by forming a vortex of liquid within the lysis tube having a mixing height of approximately 24 mm, the risk of spilling the liquid contents of the lysis tube during mixing can be reduced.

### Example 26: Onboard Pooling of 12 Samples for HTNAT

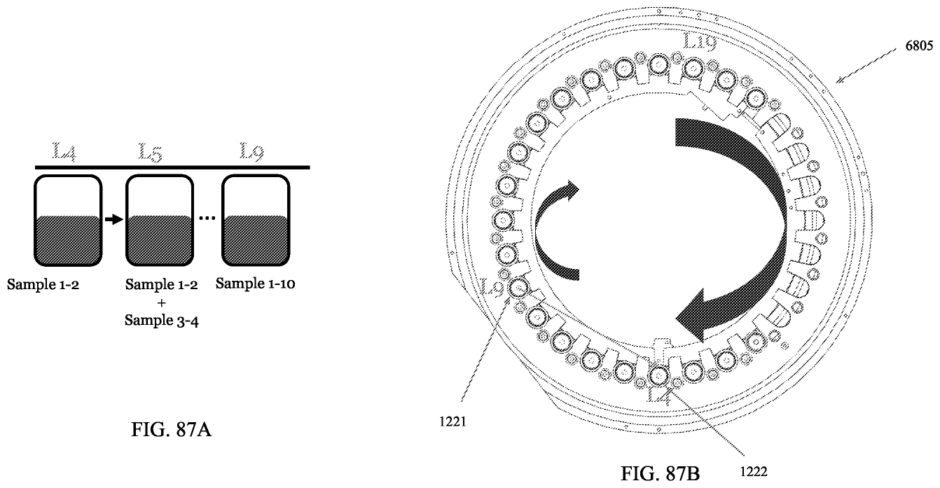

This example demonstrates an exemplary method for onboard pooling of 12 samples, such as serum or plasma samples, on a sample preparation carousel using an exemplary high-throughput nucleic acid testing (HTNAT) system such as that depicted in FIGS. 3, 62, 63, and 68A-D. Exemplary HTNAT systems such as depicted in FIGS. 3, 62, 63, and 68A-D can include a sample transport, e.g., a sample preparation carousel 6805, as described herein. With reference to the exemplary sample preparation carousel 6805 depicted in FIG. 87B, the sample preparation carousel 6805 can include 28 positions for holding vessels around an outer circumference of the sample preparation carousel. As embodied herein, the vessels can be lysis tubes. The notation L1-L21 is used herein with reference to positions on the sample preparation carousel. For purpose of this example, the carousel can be configured to move in a lockstep fashion, having about 24 seconds between movements of the carousel. As further embodied herein, a pipettor, e.g., sample/reagent pipettor 6811 can be a robotic gantry pipettor. As described above, a pipettor, e.g., sample/reagent pipettor 6811 can utilize disposable pipette tips to aspirate and dispense samples and/or pooled samples within the system.

For purpose of example, pooling of 12 samples on the sample preparation carousel 6805 can be performed as follows with reference to the exemplary HTNAT systems depicted in FIGS. 3, 62, 63, and 68A-D. A plurality of samples can be received at the sample loading area, e.g., sample loading area 3102. The plurality of samples can include at least 12 samples. The at least 12 samples can be transferred from the sample loading area to an aspiration position 6892. For example and as embodied herein, each sample can be in a tube in a sample tube rack 6802 and the sample tube rack 6802 and sample tubes therein can be moved to aspiration position 6892 using shuttles 6890, as described further herein. First and second samples can be transferred from respective sample tubes at the aspiration position 6892 to a first vessel 1221 at sample dispense position L4 on the sample preparation carousel 6805. The sample preparation carousel 6805 can then transport the first vessel 1221 to a first intermediate position L5, and third and fourth samples can be transferred from respective sample tubes at the aspiration position 6892 to the first vessel at the first intermediate position L5. The sample preparation carousel 6805 can then transport the first vessel 1221 to a second intermediate position L6 and additional samples can be transferred to the first vessel 1221 to add the additional samples to the pooled sample. This process can continue, and additional samples can be added to the pooled sample at successive intermediate positions.

For purpose of example and not limitation, the below table, Table 20, depicts an exemplary pooling process for pooling 12 samples on the sample preparation carousel 6805.

TABLE 20

| | | Running | |
|---|---|---|---|
| Position(s) | Description | Time | |
| L4 (Start) | Aspirate Sample 1 | 24 | sec |
| | Dispense Sample 1 to L4 | | |
| | Dispose of Tip | | |
| | Aspirate Sample 2 | | |
| | Dispense Sample 2 to L4 | | |
| | Dispose of Tip | | |
| L5 | Aspirate Sample 3 | 48 | sec |
| | Dispense Sample 3 to L5 | | |
| | Dispose of Tip | | |
| | Aspirate Sample 4 | | |
| | Dispense Sample 4 to L5 | | |
| | Dispose of Tip | | |
| L6 | Aspirate Sample 5 | 72 | sec |
| | Dispense Sample 5 to L6 | | |
| | Dispose of Tip | | |
| | Aspirate Sample 6 | | |
| | Dispense Sample 6 to L6 | | |
| | Dispose of Tip | | |
| L7 | Aspirate Sample 7 | 96 | sec |
| | Dispense Sample 7 to L7 | | |
| | Dispose of Tip | | |
| | Aspirate Sample 8 | | |
| | Dispense Sample 8 to L7 | | |
| | Dispose of Tip | | |
| L8 | Aspirate Sample 9 | 120 | sec |
| | Dispense Sample 9 to L8 | | |
| | Dispose of Tip | | |
| | Aspirate Sample 10 | | |
| | Dispense Sample 10 to L8 | | |
| | Dispose of Tip | | |
| L9 | Aspirate Sample 11 | 144 | sec |
| | Dispense Sample 11 to L4 | | |
| | Dispose of Tip | | |
| | Aspirate Sample 12 | | |
| | Aspirate Samples 1-10 from L9 | | |
| | Dispense Samples 1-10 and 12 to L4 | | |
| | Dispose of Tip | | |

As shown in the exemplary pooling process depicted in Table 20, two samples can be added to the pooled sample at the sample dispense position L4 and at successive intermediate positions (e.g., positions L5-L8). As further embodied herein, the pooled sample can be mixed at each intermediate position. Mixing the pooled sample can be used to achieve an even distribution of each of the individual donor samples within the pooled sample.

As embodied herein, at intermediate position L9, the pooled sample comprising samples 1-10 can be transferred from the first vessel 1221 at intermediate position L9 to a second vessel 1222 at the sample dispense position L4 of the sample preparation carousel 6805, and samples 11 and 12 can be transferred to the second vessel 1222 at the sample dispense position L4 to add samples 11 and 12 to the pooled sample. For example and as embodied herein, sample/reagent pipettor 6811 can aspirate sample 11 from the aspiration position 6892 and dispense sample 11 into the second vessel 1222 at the sample dispense position L4. Sample/reagent pipettor 6811 can further aspirate sample 12 from the aspiration position 6892, and with the same disposable pipette tip, sample/reagent pipettor 6811 can aspirate the pooled sample comprising samples 1-10 from intermediate position L9 and dispense samples 1-10 and 12 to the second vessel 1222 at the sample dispense position L4 to add samples 11 and 12 to the pooled sample.

Adding the last samples of the sample pool (e.g., samples 11 and 12 for a desired pool size of 12) directly to the second vessel 1222 can achieve a more even distribution of each of the individual donor samples within the pooled sample while minimizing the number of locksteps required to achieve the desired pooled size. For example, if samples 11 and 12 are transferred to the first vessel 1221 and the pooled sample is aspirated from the first vessel 1221 within the same lockstep, samples 11 and 12 could not have sufficient time to mix with the pooled sample prior to aspiration of the pooled sample, depending on the chosen lockstep time. For example, since pipettors sometimes do not aspirate the entire contents of a vessel, some of the pooled sample can remain in the first vessel 1221 after aspiration. Accordingly, it can be beneficial to ensure the pooled sample is adequately mixed prior to aspiration by the pipettor so that the volume of liquid aspirated can include an even distribution of the individual samples in the pooled sample.

For purpose of example and not limitation, the volume of each sample aspirated from the aspiration position 6892 and can be between about 80 and about 90 μL and the pooled sample can be between about 960 μL and about 1080 μL. As embodied herein, the volume of the pooled sample can be about 1000 μL. As embodied herein, with a desired pool sample volume of 1000 μL, the volume of each of the constituent samples used to form the pooled sample can be 1000 μL divided by the number of samples in the pooled sample (e.g., 1000 μL divided by 12 samples, about 83 μL).

As described herein, after pooling samples 1-12, additional sample preparation can be performed on the pooled sample to further prepare the pooled sample for HTNAT. For example and as embodied herein, the additional sample preparation can be a lysis process, and reagents, including lysis buffer, a protease, and CuTi-coated microparticles can be added to the second vessel 1222 and the pooled sample can be lysed in the second vessel 1222 on the sample preparation carousel as the pooled sample is continually transported along the transport path.

Nucleic acid from the pooled sample can be captured at the sample capture and transfer position L19 of the sample preparation carousel 6805 and transferred for nucleic acid analysis. For example and as embodied herein, a particle transfer mechanism 6803 including a magnetic tip can be used to capture CuTi-coated microparticles and nucleic acid bound thereto from the second vessel 1222 and transfer the CuTi-coated microparticles and nucleic acid bound thereto to for washing and elution of the nucleic acid from the pooled sample. As further embodied herein, the eluate can then be transferred to an amplification and detection system, e.g., amplification and detection system 6807, for amplification and detection of one or more of a plurality of pathogens or infectious agents in the pooled sample.

As embodied herein, 12 samples can be pooled on the sample preparation carousel in less than about 3 minutes. For purpose of example and as embodied herein, 12 samples, such as samples of plasma or serum, can be pooled on the sample preparation carousel in about 144 seconds from aspiration of the first sample from the aspiration position 6892 to dispensing of the pooled sample in the second vessel 1222 at the sample dispense position.

Exemplary HTNAT systems such as depicted in FIGS. 3, 62, 63, and 68A-D can have a throughput of approximately 150 individual donor samples of serum or plasma per hour. For purpose of example and as embodied herein, processing serum or plasm samples can include a lysis process and an amplification and detection process, as described herein. Additionally, or alternatively, exemplary HTNAT systems as depicted in FIGS. 3, 62, 63, and 68A-D can provide a time to result of about 20 to about 45 minutes for a determination of a predetermined level of nucleic acids derived from each of the plurality of pathogens or infectious agents based on the nucleic acid analysis. For purpose of example and as embodied herein, exemplary HTNAT systems as depicted in FIGS. 3, 62, 63, and 68A-D can provide a time to result of about 35 minutes when processing individual donor samples of serum or plasma.

As embodied herein, onboard sample pooling can improve sample throughput with minimal impact on time-to-result performance. For purpose of example and as embodied herein, using a pool size of 12 as described above, exemplary systems such as depicted in FIGS. 3, 62, 63, and 68A-D can have a throughput of approximately 25 pools of 12 serum or plasma sample per hour, which represents a throughput of approximately 300 individual donor samples of serum or plasma per hour and/or approximately 300 sample aspirations per hour. For purpose of example and as embodied herein, using a pool size of 12 as described above, exemplary systems such as depicted in FIGS. 3, 62, 63, and 68A-D can perform about 25 amplifications per hour. Additionally or alternatively, using a pool size of 12 as described above, exemplary systems such as depicted in FIGS. 3, 62, 63, and 68A-D can perform about 50 amplifications per hour such as by splitting the eluate containing nucleic acids from the pooled sample into two amplification vessels, as described further herein.

Additionally, or alternatively, exemplary HTNAT systems as depicted in FIGS. 3, 62, 63, and 68A-D can provide a time to result of about 20 to about 45 minutes for a determination the presence or absence of nucleic acids derived from each of the plurality of pathogens or infectious agents based on the nucleic acid analysis of the pooled sample. For purpose of example and as embodied herein, exemplary HTNAT systems as depicted in FIGS. 3, 62, 63, and 68A-D can provide a time to result of about 38 minutes when processing pools of 12 serum or plasma samples. For example and as embodied herein, onboard pooling as described herein can approximately double throughput with a minimal impact on time-to-result performance.

### Example 27: Onboard Pooling of 18 Samples for HTNAT

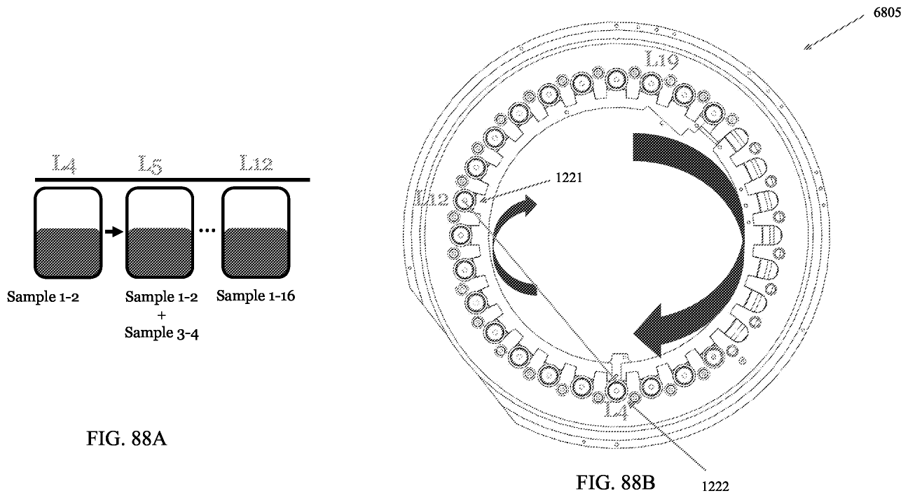

This example demonstrates an exemplary method for onboard pooling of 18 samples, such as serum or plasma samples, on a sample preparation carousel using an exemplary HTNAT system such as that depicted in FIGS. 3, 62, 63, and 68A-D. Exemplary HTNAT systems such as depicted in FIGS. 3, 62, 63, and 68A-D can include a sample preparation carousel, as described herein. With reference to the exemplary sample preparation carousel 6805 depicted in FIG. 88B, the sample preparation carousel 6805 can include 28 positions for holding vessels around an outer circumference of the sample transport. As embodied herein, the vessels can be lysis tubes. The notation L1-L21 is used herein with reference to positions on the sample preparation carousel. For purpose of this example, the carousel can be configured to move in a lockstep fashion, having about 24 seconds between movements of the carousel. As further embodied herein, a pipettor, e.g., sample/reagent pipettor 6811 can be a robotic gantry pipettor. As described above, a pipettor, e.g., sample/reagent pipettor 6811 can utilize disposable pipette tips to aspirate and transfer samples and/or pooled samples within the system.

For purpose of example, pooling of 18 samples on the sample preparation carousel 6805 can be performed as follows with reference to the exemplary HTNAT systems depicted in FIGS. 3, 62, 63, and 68A-D. A plurality of samples can be received, e.g., at aspiration position 6892 3102. The plurality of samples can include at least 18 samples. First and second samples can be transferred from respective sample tubes at the aspiration position 6892 to a first vessel 1221 at sample dispense position L4 on the sample preparation carousel 6805. The sample preparation carousel 6805 can then transport the first vessel 1221 to a first intermediate position L5, and third and fourth samples can be transferred from respective sample tubes at the aspiration position 6892 to the first vessel at the first intermediate position L5. The sample preparation carousel 6805 can then transport the first vessel 1221 to a second intermediate position L6 and additional samples can be transferred to the first vessel 1221 to add the additional samples to the pooled sample. This process can continue, and additional samples can be added to the pooled sample at successive intermediate positions.

For purpose of example and not limitation, the below table, Table 21, depicts an exemplary pooling process for pooling 18 samples on the sample preparation carousel 6805.

TABLE 21

| | | Running | |
|---|---|---|---|
| Position(s) | Description | Time | |
| L4 (Start) | Aspirate Sample 1 | 24 | sec |
| | Dispense Sample 1 to L4 | | |
| | Dispose of Tip | | |
| | Aspirate Sample 2 | | |
| | Dispense Sample 2 to L4 | | |
| | Dispose of Tip | | |
| L5 | Aspirate Sample 3 | 48 | sec |
| | Dispense Sample 3 to L5 | | |
| | Dispose of Tip | | |
| | Aspirate Sample 4 | | |
| | Dispense Sample 4 to L5 | | |
| | Dispose of Tip | | |
| L6 | Aspirate Sample 5 | 72 | sec |
| | Dispense Sample 5 to L6 | | |
| | Dispose of Tip | | |
| | Aspirate Sample 6 | | |
| | Dispense Sample 6 to L6 | | |
| | Dispose of Tip | | |
| L7 | Aspirate Sample 7 | 96 | sec |
| | Dispense Sample 7 to L7 | | |
| | Dispose of Tip | | |
| | Aspirate Sample 8 | | |
| | Dispense Sample 8 to L7 | | |
| | Dispose of Tip | | |
| L8 | Aspirate Sample 9 | 120 | sec |
| | Dispense Sample 9 to L8 | | |
| | Dispose of Tip | | |
| | Aspirate Sample 10 | | |
| | Dispense Sample 10 to L8 | | |
| | Dispose of Tip | | |
| L9 | Aspirate Sample 11 | 144 | sec |
| | Dispense Sample 11 to L9 | | |
| | Dispose of Tip | | |
| | Aspirate Sample 12 | | |
| | Dispense Sample 12 to L9 | | |
| | Dispose of Tip | | |
| L10 | Aspirate Sample 13 | 168 | sec |
| | Dispense Sample 13 to L10 | | |
| | Dispose of Tip | | |
| | Aspirate Sample 14 | | |
| | Dispense Sample 14 to L10 | | |
| | Dispose of Tip | | |
| L11 | Aspirate Sample 15 | 192 | sec |
| | Dispense Sample 15 to L11 | | |
| | Dispose of Tip | | |
| | Aspirate Sample 16 | | |
| | Dispense Sample 16 to L11 | | |
| | Dispose of Tip | | |
| L12 | Aspirate Sample 17 | 216 | sec |
| | Dispense Sample 17 to L4 | | |
| | Dispose of Tip | | |
| | Aspirate Sample 18 | | |
| | Aspirate Samples 1-16 from L12 | | |
| | Dispense Samples 1-16 and 18 to L4 | | |
| | Dispose of Tip | | |

As shown in the exemplary pooling process depicted in Table 21, two samples can be added to the pooled sample at the sample dispense position L4 and at successive intermediate positions (e.g., positions L5-L11). As further embodied herein, the pooled sample can be mixed at each intermediate position. Mixing the pooled sample can be used to achieve an even distribution of each of the individual donor samples within the pooled sample.

As embodied herein, at intermediate position L12, the pooled sample comprising samples 1-16 can be transferred from the first vessel 1221 at intermediate position L12 to a second vessel 1222 at the sample dispense position L4 of the sample preparation carousel 6805, and samples 17 and 18 can be transferred to the second vessel 1222 at the sample dispense position L4 to add samples 17 and 18 to the pooled sample. For example and as embodied herein, sample/reagent pipettor 6811 can aspirate sample 17 from the aspiration position 6892 and dispense sample 17 into the second vessel 1222 at the sample dispense position L4. Sample/reagent pipettor 6811 can further aspirate sample 18 from the aspiration position 6892, and with the same disposable pipette tip, sample/reagent pipettor 6811 can aspirate the pooled sample comprising samples 1-16 from intermediate position L12 and dispense samples 1-16 and 18 to the second vessel 1222 at the sample dispense position L4 to add samples 17 and 18 to the pooled sample. Adding the last samples of the sample pool (e.g., samples 17 and 18 for a desired pool size of 18) directly to the second vessel 1222 can achieve a more even distribution of each of the individual donor samples within the pooled sample while minimizing the number of locksteps required to achieve the desired pooled size, as described above.

For purpose of example and not limitation, the volume of each sample aspirated from the aspiration position 6892 and can be between about 50 and about 60 μL and the pooled sample can be between about 900 μL and about 1080 μL. As embodied herein, the volume of the pooled sample can be about 1000 μL. As embodied herein, with a desired pool sample volume of 1000 μL, the volume of each of the constituent samples used to form the pooled sample can be 1000 μL divided by the number of samples in the pooled sample (e.g., 1000 μL divided by 18 samples, about 56 μL).

As described herein, after pooling samples 1-18, additional sample preparation can be performed on the pooled sample to further prepare the pooled sample for HTNAT. For example and as embodied herein, the additional sample preparation can be a lysis process, and reagents, including lysis buffer, a protease, and CuTi-coated microparticles can be added to the second vessel 1222 and the pooled sample can be lysed in the second vessel 1222 on the sample preparation carousel as the pooled sample is continually transported along the transport path.

Nucleic acid from the pooled sample can be captured at the sample capture and transfer position L19 of the sample preparation carousel 6805 and transferred for nucleic acid analysis. For example and as embodied herein, a particle transfer mechanism 6803 including a magnetic tip can be used to capture CuTi-coated microparticles and nucleic acid bound thereto from the second vessel 1222 and transfer the CuTi-coated microparticles and nucleic acid bound thereto to a wash and elution system, e.g., wash and elution system 6801, for washing and elution of the nucleic acid from the pooled sample. As further embodied herein, the eluate can then be transferred to an amplification and detection system, e.g., amplification and detection system 6807, for detection of one or more of a plurality of pathogens or infectious agents in the pooled sample.

As embodied herein, 18 samples can be pooled on the sample transport in less than about 4 minutes. For purpose of example and as embodied herein, 18 samples, such as samples of plasma or serum, can be pooled on the sample transport in about 216 seconds from aspiration of the first sample from the aspiration position 6892 to dispensing of the pooled sample in the second vessel at the sample dispense position.

Exemplary HTNAT systems such as depicted in FIGS. 3, 62, 63, and 68A-D can have a throughput of approximately 150 individual donor samples of serum or plasma per hour. For purpose of example and as embodied herein, processing serum or plasm samples can include a lysis process and an amplification and detection process, as described herein. Additionally, or alternatively, exemplary HTNAT systems as depicted in FIGS. 3, 62, 63, and 68A-D can provide a time to result of about 20 to about 45 minutes for a determination of a predetermined level of nucleic acids derived from each of the plurality of pathogens or infectious agents based on the nucleic acid analysis. For purpose of example and as embodied herein, exemplary HTNAT systems as depicted in FIGS. 3, 62, 63, and 68A-D can provide a time to result of about 35 minutes when processing individual donor samples of serum or plasma.

As embodied herein, onboard sample pooling can improve sample throughput with minimal impact on time-to-result performance. For purpose of example and as embodied herein, using a pool size of 18 as described above, exemplary systems such as depicted in FIGS. 3, 62, 63, and 68A-D can have a throughput of approximately 16-17 pools of 18 serum or plasma samples per hour, which represents a throughput of approximately 288-306 individual donor samples of serum or plasma per hour. For purpose of example and as embodied herein, exemplary systems such as depicted in FIGS. 3, 62, 63, and 68A-D can have a throughput of approximately 16.7 pools of 18 serum or plasma samples per hour, which represents a throughput of approximately 300 individual donor samples of serum or plasma per hour and/or approximately 300 sample aspirations per hour. For purpose of example and as embodied herein, using a pool size of 18 as described above, exemplary systems such as depicted in FIGS. 3, 62, 63, and 68A-D can perform about 16.7 amplifications per hour. Additionally or alternatively, using a pool size of 18 as described above, exemplary systems such as depicted in FIGS. 3, 62, 63, and 68A-D can perform about 33.4 amplifications per hour such as by splitting the eluate containing nucleic acids from the pooled sample into two amplification vessels, as described further herein.

Additionally, or alternatively, exemplary HTNAT systems as depicted in FIGS. 3, 62, 63, and 68A-D can provide a time to result of about 20 to about 45 minutes for a determination of the presence or absence of nucleic acids derived from each of the plurality of pathogens or infectious agents based on the nucleic acid analysis of the pooled sample. For purpose of example and as embodied herein, exemplary HTNAT systems as depicted in FIGS. 3, 62, 63, and 68A-D can provide a time to result of about 39 minutes when processing pools of 18 serum or plasma samples. For example and as embodied herein, onboard pooling as described herein can approximately double throughput with a minimal impact on time-to-result performance.

### Example 28: Onboard Pooling of 24 Samples

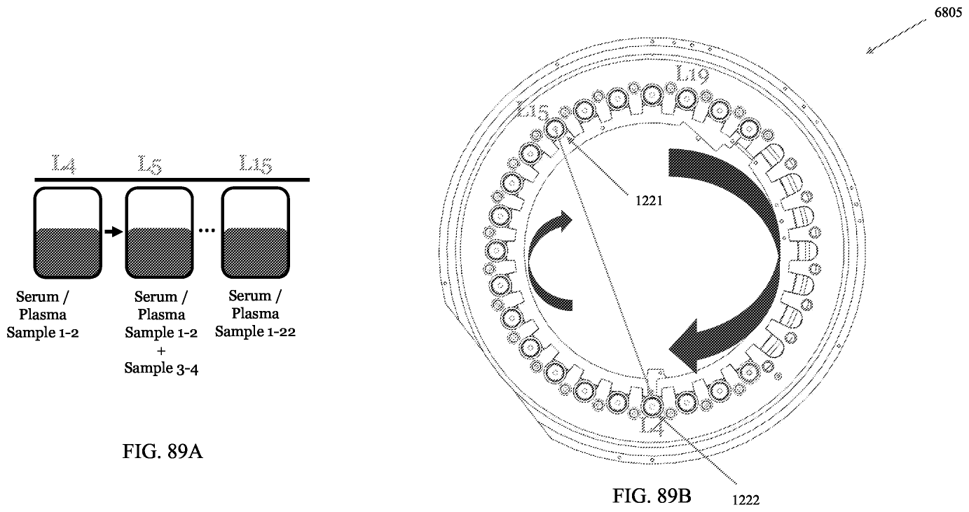

This example demonstrates an exemplary method for onboard pooling of 24 samples, such as serum or plasma samples, on a sample preparation carousel using an exemplary HTNAT system such as that depicted in FIGS. 3, 62, 63, and 68A-D. Exemplary HTNAT systems such as depicted in FIGS. 3, 62, 63, and 68A-D can include a rotatable sample preparation carousel, as described herein. With reference to the exemplary sample preparation carousel 6805 depicted in FIG. 89B, the sample preparation carousel 6805 can include 28 positions for holding vessels around an outer circumference of the sample transport. As embodied herein, the vessels can be lysis tubes. The notation L1-L21 is used herein with reference to positions on the sample preparation carousel. For purpose of this example, the carousel can be configured to move in a lockstep fashion, having about 24 seconds between movements of the carousel. As further embodied herein, a pipettor, e.g., sample/reagent pipettor 6811 can be a robotic gantry pipettor. As described above, a pipettor, e.g., sample/reagent pipettor 6811 can utilize disposable pipette tips to aspirate and transfer samples and/or pooled samples within the system.

For purpose of example, pooling of 24 samples on the sample preparation carousel 6805 can be performed as follows with reference to the exemplary HTNAT systems depicted in FIGS. 3, 62, 63, and 68A-D. A plurality of samples can be received at the sample loading area. The plurality of samples can include at least 24 samples. The at least 12 samples can be transferred from the sample loading area to an aspiration position 6892. For example and as embodied herein, each sample can be in a tube in one or more sample tube racks 6802 and the sample tube racks 6802 and sample tubes therein can be moved to aspiration position 6892 using shuttles 6890, as described further herein. First and second samples can be transferred from respective sample tubes at the aspiration position 6892 to a first vessel 1221 at sample dispense position L4 on the sample preparation carousel 6805. The sample preparation carousel 6805 can then transport the first vessel 1221 to a first intermediate position L5, and third and fourth samples can be transferred from respective sample tubes at the aspiration position 6892 to the first vessel at the first intermediate position L5. The sample preparation carousel 6805 can then transport the first vessel 1221 to a second intermediate position L6 and additional samples can be transferred to the first vessel 1221 to add the additional samples to the pooled sample. This process can continue, and additional samples can be added to the pooled sample at successive intermediate positions.

For purpose of example and not limitation, the below table, Table 22, depicts an exemplary pooling process for pooling 24 samples on the sample preparation carousel 6805.

TABLE 22

| | | Running | |
|---|---|---|---|
| Position(s) | Description | Time | |
| L4 (Start) | Aspirate Sample 1 | 24 | sec |
| | Dispense Sample 1 to L4 | | |
| | Dispose of Tip | | |
| | Aspirate Sample 2 | | |
| | Dispense Sample 2 to L4 | | |
| | Dispose of Tip | | |
| L5 | Aspirate Sample 3 | 48 | sec |
| | Dispense Sample 3 to L5 | | |
| | Dispose of Tip | | |
| | Aspirate Sample 4 | | |
| | Dispense Sample 4 to L5 | | |
| | Dispose of Tip | | |
| L6 | Aspirate Sample 5 | 72 | sec |
| | Dispense Sample 5 to L6 | | |
| | Dispose of Tip | | |
| | Aspirate Sample 6 | | |
| | Dispense Sample 6 to L6 | | |
| | Dispose of Tip | | |
| L7 | Aspirate Sample 7 | 96 | sec |
| | Dispense Sample 7 to L7 | | |
| | Dispose of Tip | | |
| | Aspirate Sample 8 | | |
| | Dispense Sample 8 to L7 | | |
| | Dispose of Tip | | |
| L8 | Aspirate Sample 9 | 120 | sec |
| | Dispense Sample 9 to L8 | | |
| | Dispose of Tip | | |
| | Aspirate Sample 10 | | |
| | Dispense Sample 10 to L8 | | |
| | Dispose of Tip | | |
| L9 | Aspirate Sample 11 | 144 | sec |
| | Dispense Sample 11 to L9 | | |
| | Dispose of Tip | | |
| | Aspirate Sample 12 | | |
| | Dispense Sample 12 to L9 | | |
| | Dispose of Tip | | |
| L10 | Aspirate Sample 13 | 168 | sec |
| | Dispense Sample 13 to L10 | | |
| | Dispose of Tip | | |
| | Aspirate Sample 14 | | |
| | Dispense Sample 14 to L10 | | |
| | Dispose of Tip | | |
| L11 | Aspirate Sample 15 | 192 | sec |
| | Dispense Sample 15 to L11 | | |
| | Dispose of Tip | | |
| | Aspirate Sample 16 | | |
| | Dispense Sample 16 to L11 | | |
| | Dispose of Tip | | |
| L12 | Aspirate Sample 17 | 216 | sec |
| | Dispense Sample 17 to L12 | | |
| | Dispose of Tip | | |
| | Aspirate Sample 18 | | |
| | Dispense Sample 18 to L12 | | |
| | Dispose of Tip | | |
| L13 | Aspirate Sample 19 | 240 | sec |
| | Dispense Sample 19 to L13 | | |
| | Dispose of Tip | | |
| | Aspirate Sample 20 | | |
| | Dispense Sample 20 to L13 | | |
| | Dispose of Tip | | |
| L14 | Aspirate Sample 21 | 264 | sec |
| | Dispense Sample 21 to L14 | | |
| | Dispose of Tip | | |
| | Aspirate Sample 22 | | |
| | Dispense Sample 22 to L14 | | |
| | Dispose of Tip | | |
| L15 | Aspirate Sample 23 | 288 | sec |
| | Dispense Sample 23 to L4 | | |
| | Dispose of Tip | | |
| | Aspirate Sample 24 | | |
| | Aspirate Samples 1-22 from L15 | | |
| | Dispense Samples 1-22 and 24 to L4 | | |
| | Dispose of Tip | | |

As shown in the exemplary pooling process depicted in Table 22, two samples can be added to the pooled sample at the sample dispense position L4 and at successive intermediate positions (e.g., positions L5-L14). As further embodied herein, the pooled sample can be mixed at each intermediate position. Mixing the pooled sample can be used to achieve an even distribution of each of the individual donor samples within the pooled sample.

As embodied herein, at intermediate position L15, the pooled sample comprising samples 1-22 can be transferred from the first vessel 1221 at intermediate position L12 to a second vessel 1222 at the sample dispense position L4 of the sample preparation carousel 6805, and samples 23 and 24 can be transferred to the second vessel 1222 at the sample dispense position L4 to add samples 23 and 24 to the pooled sample. For example and as embodied herein, sample/reagent pipettor 6811 can aspirate sample 23 from the aspiration position 6892 and dispense sample 23 into the second vessel 1222 at the sample dispense position L4. Sample/reagent pipettor 6811 can further aspirate sample 24 from the aspiration position 6892, and with the same disposable pipette tip, sample/reagent pipettor 6811 can aspirate the pooled sample comprising samples 1-22 from intermediate position L15 and dispense samples 1-22 and 24 to the second vessel 1222 at the sample dispense position L4 to add samples 23 and 24 to the pooled sample. Adding the last samples of the sample pool (e.g., samples 23 and 24 for a desired pool size of 24) directly to the second vessel 1222 can achieve a more even distribution of each of the individual donor samples within the pooled sample while minimizing the number of locksteps required to achieve the desired pooled size, as described above.

For purpose of example and not limitation, the volume of each sample aspirated from the aspiration position 6892 and can be between about 35 and about 45 μL and the pooled sample can be between about 840 μL and about 1080 μL. As embodied herein, the volume of the pooled sample can be about 1000 μL. As embodied herein, with a desired pool sample volume of 1000 μL, the volume of each of the constituent samples used to form the pooled sample can be 1000 μL divided by the number of samples in the pooled sample (e.g., 1000 μL divided by 24 samples, about 42 μL).

As described herein, after pooling samples 1-24, additional sample preparation can be performed on the pooled sample to further prepare the pooled sample for HTNAT. For example and as embodied herein, the additional sample preparation can be a lysis process, and reagents, including lysis buffer, a protease, and CuTi-coated microparticles can be added to the second vessel 1222 and the pooled sample can be lysed in the second vessel 1222 on the sample preparation carousel as the pooled sample is continually transported along the transport path.

Nucleic acid from the pooled sample can be captured at the sample capture and transfer position L19 of the sample preparation carousel 6805 and transferred for nucleic acid analysis. For example and as embodied herein, a particle transfer mechanism 6803 including a magnetic tip can be used to capture CuTi-coated microparticles and nucleic acid bound thereto from the second vessel 1222 and transfer the CuTi-coated microparticles and nucleic acid bound thereto to a wash and elution system, e.g., wash and elution system 6801, for washing and elution of the nucleic acid from the pooled sample. As further embodied herein, the eluate can then be transferred to an amplification and detection system, e.g., amplification and detection system 6807, for amplification and detection of one or more of a plurality of pathogens or infectious agents in the pooled sample.

As embodied herein, 24 samples can be pooled on the sample transport in less than about 5 minutes. For purpose of example and as embodied herein, 24 samples, such as samples of plasma or serum, can be pooled on the sample transport in about 288 seconds from aspiration of the first sample from the aspiration position 6892 to dispensing of the pooled sample in the second vessel at the sample dispense position.

Exemplary HTNAT systems such as depicted in FIGS. 3, 62, 63, and 68A-D can have a throughput of approximately 150 individual donor samples of serum or plasma per hour. For purpose of example and as embodied herein, processing serum or plasm samples can include a lysis process and an amplification and detection process, as described herein. Additionally, or alternatively, exemplary HTNAT systems as depicted in FIGS. 3, 62, 63, and 68A-D can provide a time to result of about 20 to about 45 minutes for a determination of a predetermined level of nucleic acids derived from each of the plurality of pathogens or infectious agents based on the nucleic acid analysis. For purpose of example and as embodied herein, exemplary HTNAT systems as depicted in FIGS. 3, 62, 63, and 68A-D can provide a time to result of about 35 minutes when processing individual donor samples of serum or plasma.

As embodied herein, onboard sample pooling can improve sample throughput with minimal impact on time-to-result performance. For purpose of example and as embodied herein, using a pool size of 24 as described above, exemplary systems such as depicted in FIGS. 3, 62, 63, and 68A-D can have a throughput of approximately 12-13 pools of 24 serum or plasma samples per hour, which represents a throughput of approximately 288-312 individual donor samples of serum or plasma per hour. For purpose of example and as embodied herein, exemplary systems such as depicted in FIGS. 3, 62, 63, and 68A-D can have a throughput of approximately 12.5 pools of 24 serum or plasma samples per hour, which represents a throughput of approximately 300 individual donor samples of serum or plasma per hour and/or approximately 300 sample aspirations per hour. For purpose of example and as embodied herein, using a pool size of 24 as described above, exemplary systems such as depicted in FIGS. 3, 62, 63, and 68A-D can perform about 12.5 amplifications per hour. Additionally or alternatively, using a pool size of 24 as described above, exemplary systems such as depicted in FIGS. 3, 62, 63, and 68A-D can perform between about 25 amplifications per hour such as by splitting the eluate containing nucleic acids from the pooled sample into two amplification vessels, as described further herein.

Additionally, or alternatively, exemplary HTNAT systems as depicted in FIGS. 3, 62, 63, and 68A-D can provide a time to result of about 20 to about 45 minutes for a determination of the presence or absence of nucleic acids derived from each of the plurality of pathogens or infectious agents based on the nucleic acid analysis of the pooled sample. For purpose of example and as embodied herein, exemplary HTNAT systems as depicted in FIGS. 3, 62, 63, and 68A-D can provide a time to result of about 39 minutes when processing pools of 24 serum or plasma samples. For example and as embodied herein, onboard pooling as described herein can approximately double throughput with a minimal impact on time-to-result performance.

### Example 29: Onboard Lysis Pre-Treatment and Pooling of 6 Sample Lysates

This example demonstrates an exemplary method for onboard lysis pre-treatment and pooling of 6 sample lysates on a sample transport using an exemplary HTNAT system such as that depicted in FIGS. 3, 62, 63, and 68A-D. For example and as embodied herein, whole blood samples can be pre-treated and the whole blood lysate can be pooled.

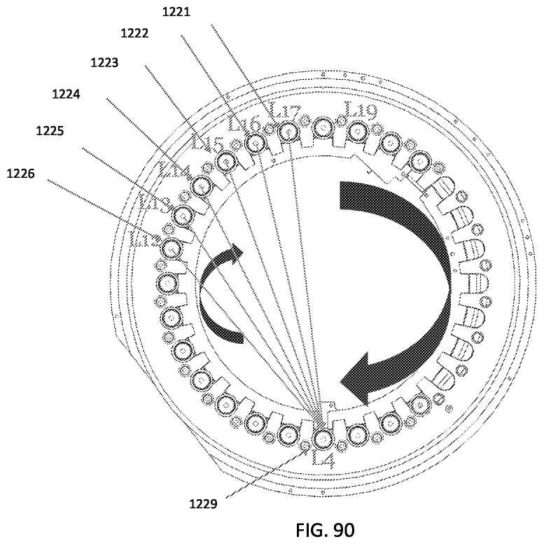

Exemplary HTNAT systems such as depicted in FIGS. 3, 62, 63, and 68A-D can include a rotatable sample preparation carousel, as described herein. With reference to the exemplary sample preparation carousel 6805 depicted in FIG. 90, the sample preparation carousel 6805 can include 28 positions for holding vessels around an outer circumference of the sample transport. As embodied herein, the vessels can be lysis tubes. The notation L1-L21 is used herein with reference to positions on the sample preparation carousel. For purpose of this example, the carousel can be configured to move in a lockstep fashion, having about 24 seconds between movements of the carousel. As further embodied herein, a pipettor, e.g., sample/reagent pipettor 6811 can be a robotic gantry pipettor. As described above, a pipettor, e.g., sample/reagent pipettor 6811 can utilize disposable pipette tips to aspirate and transfer samples and/or pooled samples within the system.

For purpose of example, lysis pre-treatment and pooling of 6 sample lysates on the sample preparation carousel 6805 can be performed as follows with reference to the exemplary HTNAT system 100 depicted in FIGS. 3, 62, 63, and 68A-D. A plurality of samples can be received at the sample loading area. The plurality of samples can include at least 6 samples. A first sample can be transferred from a first sample tube at the aspiration position 6892 to a first vessel 1221 at sample dispense position L4 on the sample preparation carousel 6805. The sample preparation carousel 6805 can then transport the first vessel 1221 to a first intermediate position L5. A second sample can be transferred from a second sample tube at the aspiration position 6892 to a second vessel 1222 at the sample dispense position L4 on the sample preparation carousel 6805. The sample preparation carousel 6805 can then transport the second vessel 1222 to the first intermediate position L5 and the first vessel 1221 to a second intermediate position L6. This process can continue, and additional samples can be added to additional vessels at the sample dispense position L4 as the sample preparation carousel 6805 continually transports individual vessels along the transport path.

For purpose of example and as embodied herein, lysis buffer can be added to the first vessel 1221, second vessel 1222 and additional vessels for samples 3-6, and samples 1-6 can lyse within their respective vessel as the sample preparation carousel 6805 continually transports the vessels along the transport path. As further embodied herein, samples 1-6 can be mixed at each intermediate position. Mixing the samples as they are transported along the transport path can be beneficial, for example, to mix the sample and lysis buffer, which can facilitate lysis of the samples.

For purpose of example and not limitation, the below table, Table 23, depicts an exemplary process for pre-treatment and pooling of sample lysates on the sample preparation carousel 6805.

TABLE 23

| Position(s) | Description | Time | |
|---|---|---|---|
| L4 (Start) | Dispense Sample | 24 | sec |
| | Dispose of Tip | | |
| L5-L16 | Incubation and Mixing | 312 | sec |

| | | |
|---|---|---|
| L17 | Aspirate Sample 1 from L17 | 336 |

Aspirate Sample 2 from L16
Aspirate Sample 3 from L15
Aspirate Sample 4 from L14
Aspirate Sample 5 from L13
Aspirate Sample 6 from L12
Dispense Samples 1-6 to L4
Dispose of Tip

As shown in the exemplary pre-treatment and pooling process depicted in Table 23 and FIG. 90, when the first sample in the first vessel 1221 reaches intermediate position L17, the first sample can be transferred from the first vessel 1221 at intermediate position L17 to a new vessel 1229 at the sample dispense position L4 and the second sample in the second vessel 1222 can be transferred from the second vessel 1222 at intermediate position L16 to the new vessel 1229 to pool the first and second sample lysates. Additionally, samples 3-6 can be transferred from their respective vessels and respective intermediate positions to the new vessel at the sample dispense position to add the sample 3-6 lysates to the pooled sample. For purpose of example and as embodied herein, samples 1-6 can be aspirated from their respective vessels and intermediate positions using one disposable tip and samples 1-6 can be dispensed into the new vessel at the sample dispense position in one dispense operation. Additionally or alternatively, samples 1-6 can be aspirated from their respective vessels and intermediate positions using separate tips and/or separate aspirations and dispenses.

For purpose of example and as embodied herein, during the pre-treatment, samples 1-6 can be allowed to lyse on the sample transport for between about 3 and about 6 minutes as the sample transport continually transports the vessels along the transport path. For purpose of example and as embodied herein, the first sample can be lysed for between about 5 and about 6 minutes, and the sixth sample can be lysed for between about 3 and about 4 minutes.

For purpose of example and not limitation, the volume of each of the pre-treated samples aspirated from the respective intermediate position and can be between about 45 and about 55 μL and the pooled sample can include between about 270 μL and about 330 μL of the pooled pre-treated samples. As embodied herein, the volume of the pooled pre-treated sample can be about 300 μL. As embodied herein, with a desired pooled pre-treated sample volume of 300 μL, the volume of each of the constituent pre-treated samples used to form the pooled sample can be 300 μL divided by the number of pre-treated samples in the pooled sample (e.g., 300 μL divided by 6 samples).

As described herein, after pooling samples 1-6, additional sample preparation can be performed on the pooled sample to further prepare the pooled sample for HTNAT. For example and as embodied herein, the additional sample preparation can be a lysis process, and reagents, including lysis buffer, a protease, and CuTi-coated microparticles can be added to the new vessel 1229 and the pooled sample can be lysed in the new vessel 1229 on the sample preparation carousel as the pooled sample is continually transported along the transport path.

Nucleic acid from the pooled sample can be captured at the sample capture and transfer position L19 of the sample preparation carousel 6805 and transferred for nucleic acid analysis. For example and as embodied herein, a particle transfer mechanism 6803 including a magnetic tip can be used to capture CuTi-coated microparticles and nucleic acid bound thereto from the new vessel 1229 and transfer the CuTi-coated microparticles and nucleic acid bound thereto to a wash and elution system, e.g., wash and elution system 6801, for washing and elution of the nucleic acid from the pooled sample. As further embodied herein, the eluate can then be transferred to an amplification and detection system, e.g., amplification and detection system 6807, for amplification and detection of one or more of a plurality of pathogens or infectious agents in the pooled sample.

As embodied herein, 6 samples can be pre-treated and pooled on the sample transport in less than about 6 minutes. For purpose of example and as embodied herein, 6 samples, such as whole blood samples, can be lysed on the sample transport and the lysates from the 6 samples can be pooled in about 336 seconds from aspiration of the first sample from the aspiration position 6892 to dispensing of the lysates in the new vessel 1229 at the sample dispense position.

Exemplary HTNAT systems such as depicted in FIGS. 3, 62, 63, and 68A-D can have a throughput of approximately 75 individual donor samples of whole blood per hour when the whole blood samples are pre-treated onboard but not pooled onboard. For purpose of example and as embodied herein, processing whole blood samples can include a pretreatment process, a lysis process, and an amplification and detection process, as described herein. Additionally, or alternatively, exemplary HTNAT systems as depicted in FIGS. 3, 62, 63, and 68A-D can provide a time to result of about 20 to about 45 minutes for a determination of a predetermined level of nucleic acids derived from each of the plurality of pathogens or infectious agents based on the nucleic acid analysis. For purpose of example and as embodied herein, exemplary HTNAT systems as depicted in FIGS. 3, 62, 63, and 68A-D can provide a time to result of about 42 minutes when processing individual donor samples of whole blood, including onboard pre-treatment.

As embodied herein, onboard pre-treatment and sample pooling can improve sample throughput with minimal impact on time-to-result performance. For purpose of example and as embodied herein, using a pool size of 6 as described above, exemplary systems such as depicted in FIGS. 3, 62, 63, and 68A-D can have a throughput of approximately 19-23 pools of 6 whole blood samples per hour, which represents a throughput of approximately 114-138 individual donor samples of whole blood per hour. For purpose of example and as embodied herein, exemplary systems such as depicted in FIGS. 3, 62, 63, and 68A-D can have a throughput of approximately 21 pools of 6 whole blood samples per hour, which represents a throughput of approximately 126 individual donor samples of serum or plasma per hour and/or approximately 126 sample aspirations per hour. For purpose of example and as embodied herein, using a pool size of 6 as described above, exemplary systems such as depicted in FIGS. 3, 62, 63, and 68A-D can perform about 21 amplifications per hour. Additionally or alternatively, using a pool size of 6 as described above, exemplary systems such as depicted in FIGS. 3, 62, 63, and 68A-D can perform between about 42 amplifications per hour such as by splitting the eluate containing nucleic acids from the pooled sample into two amplification vessels, as described further herein.

Additionally, or alternatively, exemplary HTNAT systems as depicted in FIGS. 3, 62, 63, and 68A-D can provide a time to result of about 20 to about 45 minutes for a determination of the presence or absence of nucleic acids derived from each of the plurality of pathogens or infectious agents based on the nucleic acid analysis of the pooled sample.

For purpose of example and as embodied herein, exemplary HTNAT systems as depicted in FIGS. 3, 62, 63, and 68A-D can provide a time to result of about 42 minutes when processing pools of 6 whole blood samples. For example and as embodied herein, onboard pooling as described herein can increase throughput with a minimal impact on time-to-result performance.

### Example 30: Pooling with Liquid Handler and Onboard Pooling

This example demonstrates an exemplary method for pooling samples using both conventional pooling, such as on a dedicated liquid handler or pooler, and onboard pooling. For purpose of example and as described herein, using conventional pooling with a separate liquid handler and onboard pooling in combination can improve laboratory efficiency and throughput.

As embodied herein, a pooled sample of 96 samples can be formed using onboard pooling of first and second samples on an exemplary HTNAT system such as that depicted in FIGS. 3, 62, 63, and 68A-D. For purpose of this example, the first and second samples can each be pools of 48 samples. As embodied herein, the first and second samples can be prepared on a dedicated liquid handler.

Onboard pooling of the first and second samples as embodied herein includes receiving the first and second samples at a aspiration position 6892 of the system and transferring the first and second samples from respective sample containers at the aspiration position 6892 to a vessel at the sample dispense position L4 on sample preparation carousel 6805. As embodied herein, the vessel can be a lysis tube. As embodied herein, pooling the first and second sample can include transferring the first sample and the second sample to a first vessel 1221 at the sample dispense position L4 prior to the sample transport transporting the first vessel 1221 to a first intermediate position L5. The pooled sample can then be transported along the transport path to the sample capture and transfer position, where a particle transfer mechanism, e.g., particle transfer mechanism 6803, can be used to capture CuTi-coated microparticles and nucleic acid bound thereto from the first vessel 1221 and transfer the CuTi-coated microparticles and nucleic acid bound thereto to a wash and elution system, e.g., wash and elution system 6801, for washing and elution of the nucleic acid from the pooled sample. As further embodied herein, the eluate can then be transferred to an amplification and detection system, e.g., amplification and detection system 6807, for amplification and detection of one or more of a plurality of pathogens or infectious agents in the pooled sample of 96.

For purpose of background and illustration, in a conventional plasma laboratory, liquid handler pooling throughput, not instrument throughput, can be the limiting factor. For example, plasma labs often use pool sizes of 96 samples, which can be prepared on 8-channel liquid handlers. The ratio of liquid handlers to testing instruments, such as NAT instruments, can be as high as 8 liquid handlers to 1 testing instrument. For purpose of example and illustration, conventional NAT platforms used in plasma labs can process up to 4,032 tests per day on pooled samples of 96 prepared on a traditional liquid handler, and plasma labs can run 2 tests per pooled sample, so a single conventional NAT instrument can process approximately 193,536 samples per day (e.g., [(4,032 tests)/(2 tests per pooled sample)]*96 samples per pool=193,536 samples per day).

For purpose of background and illustration, conventional liquid handlers often use an 8-channel pipettor, and during pooling, 768 samples are placed on the liquid handler and pipetted, 8 at a time, into 8 master pool tubes. After 96 cycles, 8 pools of 96 can be prepared and can be moved to an instrument for analysis. This can be the most efficient method to create pools of 96 on a conventional liquid handler. For example, when pooling on a liquid handler, the number of pools created can be a whole number multiple of the number of pipetting channels of the liquid handler to minimize the number of pipetting cycles need to form each pool and to maximize the use of the available pipette channels.

Although 8-channel pipettors are common on conventional liquid handlers, conventional liquid handlers are available in configurations up to 16-channels. However, 16-channel liquid handlers are often not used when forming pools of 96 due to the size of conventional liquid handlers. For example, many liquid handlers cannot hold the 1,536 samples needed to create 16 pools of 96 using a 16-channel pipettor. As a result, the 16-channel liquid handlers can be limited to creating 16 pools of 48 with 768 samples due to restrictions on the number of samples that the liquid handler can hold. The pooling throughput of 16-channel liquid handlers can be double the throughput of the conventional 8-channel configuration. For example, 768 samples can be transferred into 16 pools of 48 with 48 moves of a 16-channel pipettor, whereas 768 samples can be transferred into 8 pools of 96 with 96 moves of an 8-channel pipettor. However, plasma labs generally prefer to use larger pool sizes as the cost of testing can be reduced with larger pool sizes. pools of 96 can be preferred over pools of 48. For example, plasma laboratories often prefer pools of 96 as compared to pools of 48.

As embodied herein, using a combination of conventional pooling with a 16-channel liquid handler and onboard pooling, can increase overall laboratory pooling throughput and can reduce the number of liquid handlers required to support each testing instrument. For example, by using onboard pooling to form a pool of 96 by pooling first and second samples comprising pools of 48 prepared on a liquid handler, 16-channel liquid-handler configurations can be used, which can double laboratory pooling throughput and can half the ratio of liquid handlers to instruments to 4:1, while maintaining a pool size of 96.

Although this example has been described in the context of onboard pooling of 2 pools of 48 prepared on a liquid handler to form a pool of 96, it is to be understood that additional or alternative configurations can be used. For example and not limitation, two or more pools of 48 prepared on a liquid handler can be pooled onboard to create larger pools, such as pools of 144 or pools of 192 or pools of 240.

### Example 31: Pooling with Liquid Handler and Onboard Pooling and Onboard Deconstruction

This example demonstrates an exemplary method for onboard deconstruction of a pooled sample. For example, when a pooled sample is determined to be positive, or reactive, the pooled sample can be deconstructed to identify which sample or samples used to form the pooled sample are positive or reactive. For purpose of example and as embodied herein, HTNAT can be performed on the pooled sample, and a positive pooled sample can be identified based upon a determination of the presence of a nucleic acid derived from at least one of a plurality of pathogens or infectious agents in the pooled sample. As embodied herein, when it is determined that a pooled sample is positive, further nucleic acid analysis can be performed on the individual samples or sub-pools thereof that were used to form the positive pooled sample.

This example demonstrates an exemplary method for onboard deconstruction of pooled sample of 96 plasma samples using an exemplary HTNAT system such as that depicted in FIGS. 3, 62, 63, and 68A-D. As described above, a pool of 96 samples can be formed, for example, using both conventional pooling, such as on a dedicated liquid handler or pooler, and onboard pooling. For example, and as embodied herein, a first sample comprising a pool of 48 and a second sample comprising a pool of 48 can be prepared on a dedicated liquid handler and the first and second samples can then be transferred to the sample loading area of a system for onboard pooling and high-throughput analysis, such as the exemplary HTNAT system depicted in FIGS. 3, 62, 63, and 68A-D. As embodied herein, the first sample and the second sample used to form the pooled sample can be stored at the sample loading area while high-throughput analysis is performed on the pooled sample. Onboard pooling and HTNAT analysis of the pooled sample can be performed using the exemplary HTNAT system depicted in FIGS. 3, 62, 63, and 68A-D and methods as described herein.

As embodied herein, upon a determination of the presence of a nucleic acid derived from at least one of a plurality of pathogens or infectious agents in the pooled sample of 96, the first sample and the second sample, each comprising a pool of 48 samples, can automatically be transferred from the sample loading area to the sample preparation carousel 6805 for further nucleic acid analysis. For example and as embodied herein, the first and second samples can be transferred to the sample preparation carousel 6805 for sample preparation and amplification and detection to determine the presence of a nucleic acid derived from at least one of a plurality of pathogens or infectious agents in the first sample or the second sample. Based on the results of the nucleic acid analysis of the first sample and the second sample, it can be determined whether the first sample, the second sample, or both the first and second sample include nucleic acid derived from at least one of a plurality of pathogens or infectious agents.

The positive first and/or second sample can then be further deconstructed. For example and as embodied herein, the 48 individual samples used to form the positive first or second sample can then be transferred to the system for nucleic acid analysis. Transferring the 48 individual samples to the system can be performed by an operator, or can be performed automatically such as by a laboratory automation system. The 48 individual samples can then be stored at the sample loading area while further deconstruction of the positive first and/or second sample is performed. Onboard pooling and nucleic acid analysis of the 48 individual samples can then be performed as described herein. For example and as embodied herein, deconstructing the positive first or second sample can include onboard pooling to form three sub-pools of 16 individual samples and nucleic acid analysis of the three sub-pools to determine which sub-pool is positive. As embodied herein, each of the three sub-pools can contain 16 individual samples representing a subset of the individual samples that had been used to form the positive first or second sample. Onboard pooling and nucleic acid analysis of the sub-pools can be performed using the exemplary HTNAT system depicted in FIGS. 3, 62, 63, and 68A-D and methods as described herein. Based on the results of the nucleic acid analysis of the three sub-pools, it can be determined which sub-pool includes nucleic acid derived from at least one of a plurality of pathogens or infectious agents.

As embodied herein, the positive sub-pool(s) can then be further deconstructed. As embodied herein, deconstructing the positive sub-pool can include onboard pooling to form four sub-pools of 4 individual samples and performing nucleic acid analysis on each of the four sub-pools of 4 individual samples to determine which of the sub-pools is positive. As embodied herein, each of the four sub-pools can contain 4 individual samples, representing a subset of the individual samples that had been used to form the positive sub pool of 16 individual samples. Onboard pooling and nucleic acid analysis of the sub-pools can be performed using the exemplary HTNAT system depicted in FIGS. 3, 62, 63, and 68A-D and methods as described herein. Based on the results of the nucleic acid analysis of the four sub-pools, it can be determined which of the four sub-pools includes nucleic acid derived from at least one of a plurality of pathogens or infectious agents.

As embodied herein, the positive sub-pool of four individual samples can then be further deconstructed. As embodied herein, deconstructing the positive sub-pool of four individual samples can include performing nucleic acid analysis of the four individual samples that had been used to form the positive sub-pool. Nucleic acid analysis of the individual samples can be performed using the exemplary HTNAT system depicted in FIGS. 3, 62, 63, and 68A-D and methods as described herein. Based on the results of the nucleic acid analysis of the four individual samples, it can be determined which of the four individual samples includes nucleic acid derived from at least one of a plurality of pathogens or infectious agents.

The systems and methods described herein can reduce the number of manual steps and the amount of time needed to deconstruct a pooled sample. For example and illustration, deconstructing a positive pool of 96 samples using conventional methods can take 12 hours or longer and can require multiple manual steps. For example, in conventional methods, when a positive pool of 96 is identified, a user can manually retrieve the 96 constituent samples and place them back on a liquid handler to be pooled into 16 sub-pools of 6. These 16 sub-pools can then manually be transported from the liquid handler to an instrument for analysis. Analysis of the 16 sub-pools can take up to 3.5-4 hours, after which the reactive sub-pool(s) can then be deconstructed. In conventional methods, the 6 samples in each reactive sub-pool are not stored on the instrument during testing, and must be retrieved and placed back on the instrument for individual testing (IDT). IDT of the 6 samples in each reactive sub-pool can take an additional 3.5-4 hours to determine which of the individual sample(s) from the pooled sample are reactive. Using conventional methods, deconstructing a pool of 96 samples can take over 12 hours.

In certain embodiments, the systems and methods disclosed herein can reduce the amount of time and the number of manual steps for pool deconstruction. For example, and as embodied herein, based on the results of the nucleic acid analysis, a determination can be made in less than about 4 hours as to which of the 96 individual samples used to form the pooled sample includes nucleic acid derived from at least one of a plurality of pathogens or infectious agents. As further embodied herein, a determination as to whether donor material associated with each of the 96 individual donor samples used to form the pooled sample is acceptable for clinical use can be made in less than about 4 hours from initial aspiration of the first sample for nucleic acid analysis.

1-503. (canceled)

504. A method for screening a plurality of samples to determine whether donor blood associated with the samples is acceptable for transfusion comprising:
performing a nucleic acid analysis on each sample of the plurality of samples, wherein the nucleic acid analysis comprises a sample preparation process, an amplification process, and a detection process;
determining a result from the nucleic acid analysis, wherein the result includes a determination of a predetermined level of nucleic acid from at least one of a plurality of pathogens or infectious agents, and wherein each result has a time to result of about 20 to about 45 minutes; and
determining whether the donor blood is acceptable for transfusion based at least in part on the nucleic acid analysis result.

505. The method of claim 504, wherein the nucleic acid analysis and the determining a result from the nucleic acid analysis are performed on an automated system.

506. The method of claim 505, wherein at least about 140 results are obtained per hour per m2 of a footprint occupied by the automated system.

507. The method of claim 505, wherein at least about 70 results are obtained per hour per m3 of a volume occupied by the automated system.

508. The method of claim 504, wherein the plurality of pathogens or infectious agents are selected from the group consisting of: HIV-1, HIV-2, HBV, HCV, Parvovirus B19, HAV, WNV, Zika Virus, Dengue Virus, Chikungunya Virus, Babesia, Malaria, and HEV.

509. The method of claim 504, wherein the determination of a level less than the predetermined level indicates that the donor blood is acceptable for transfusion.

510. The method of claim 504 wherein the plurality of pathogens or infectious agents and predetermined levels are selected from the following:
HIV-1 at a predetermined level of at least 1-50 copies/mL;
HIV-2 at a predetermined level of at least 1-20 IU/mL;
HBV at a predetermined level of at least 1-10 IU/mL;
HCV at a predetermined level of at least 1-50 IU/mL;
Parvovirus B19 at a predetermined level of at least 1-40 IU/mL;
HAV at a predetermined level of at least 1-10 IU/mL;
WNV at a predetermined level of at least 1-50 copies/mL;
Zika Virus at a predetermined level of at least 1-50 copies/mL;
Dengue Virus at a predetermined level of at least 1-50 copies/mL;
Chikungunya Virus at a predetermined level of at least 1-50 copies/mL;
Babesia at a predetermined level of at least 1-20 copies/mL;
Malaria at a predetermined level of at least 1-50 copies/mL; and
HEV at a predetermined level of at least 1-20 IU/mL.

511. The method of claim 504, wherein:
the plurality of pathogens or infectious agents are HIV-1, HIV-2, HCV, and HBV; and
the nucleic acid analysis comprises multiplex analysis of HIV-1, HIV-2, HCV, and HBV.

512. The method of claim 504, wherein:
the plurality of pathogens or infectious agents comprise Zika Virus, WNV, Chikungunya Virus and Dengue Virus;
the nucleic acid analysis comprises multiplex analysis of Zika Virus and Dengue; and
the nucleic acid analysis comprises multiplex analysis of Chikungunya Virus and WNV.

513. The method of claim 504, wherein:
the plurality of pathogens or infectious agents comprise Parvovirus B19 and HAV; and
the nucleic acid analysis comprises multiplex analysis of Parvovirus B19 and HAV.

514. The method of claim 504, wherein at least one of the plurality of samples of donor blood is a pooled sample.

515. The method of claim 504, wherein the sample preparation process comprises onboard pooling of a subset of the plurality of samples to form a pooled sample, the nucleic acid analysis being performed on the pooled sample.

516. The method of claim 504, wherein the sample preparation process comprises preparing an eluate and wherein the eluate is split into two or more amplification vessels.

517. The method of claim 504, wherein the sample preparation process comprises a lysis process and a wash process.

518. The method of claim 517, wherein the lysis process is completed in about 4 to about 8 minutes.

519. The method of claim 517, wherein the wash process is completed in about 5 to about 9 minutes.

520. The method of claim 517, wherein the wash process comprises at least one wash step and an elution step.

521. The method of claim 504, wherein the sample preparation process is completed in about 12 to about 16 minutes.

522. The method of claim 504, wherein the amplification process and the detection process are performed together in an amplification and detection process.

523. The method of claim 522, wherein the amplification and detection process is completed in about 1 to about 22 minutes.

524. The method of claim 504, wherein the time to result begins from initial aspiration of the sample for performing the nucleic acid analysis and/or ends with the determination of the result.

525. The method of claim 505, wherein the automated system comprises a sample analysis station comprising:
a sample loading area;
a sample preparation area for performing the sample preparation process;
a nucleic acid amplification area for performing the amplification process; and
a nucleic acid detection area for performing the detection process.

526. The method of claim 525, wherein the nucleic acid amplification area and the nucleic acid detection area is a single area.

527. The method of claim 526, wherein the single area includes an amplification and detection system configured to perform the amplification process and the detection process simultaneously.

528. The method of claim 525, wherein the sample preparation area includes a sample preparation carousel and/or a wash and elution system.


SEQUENCE LISTING (124 sequences)

SEQ ID NO: 1 - DNA, 32 bp, Artificial Sequence
  Synthetic HCV NEAR T1
  misc_feature (1)..(32): contains MeOU modification; see specification as filed for detailed description of modifications
cgactccatatggagtcgtgtgggtagcgaaa

SEQ ID NO: 2 - DNA, 32 bp, Artificial Sequence
  Synthetic HCV NEAR T2
  misc_feature (1)..(32): contains MeOC modification; see specification as filed for detailed description of modifications
tgactccatatggagtcagcacctatcaggca

SEQ ID NO: 3 - DNA, 26 bp, Artificial Sequence
  Synthetic HCV NEAR MB
  misc_feature (1)..(26): contains P2, FAM, BHQ1 modifications; see specification as filed for detailed description of modifications
cgtaccgcgaaaggccttgtggtacg

SEQ ID NO: 4 - DNA, 32 bp, Artificial Sequence
  Synthetic CHIKV_E1_F7
agctgtaaggtcttcaccggcgtctacccatt

SEQ ID NO: 5 - DNA, 32 bp, Artificial Sequence
  Synthetic CHIKV_E1_R7
gtatgagccctgtatgctgatgcaaattctgt

SEQ ID NO: 6 - DNA, 32 bp, Artificial Sequence
  Synthetic CHIKV_E1_R8
gcggtatgagccctgtatgctgatgcaaattc

SEQ ID NO: 7 - DNA, 49 bp, Artificial Sequence
  Synthetic CHIKV_E1_P3
  misc_feature (1)..(49): contains FAM, THF, BHQ1 modifications; see specification as filed for detailed description of modifications
caccgaaaatacgcaattgagcgaagcacattggagaagtccgaatcat

SEQ ID NO: 8 - DNA, 30 bp, Artificial Sequence
  Synthetic DENV 4 FP
cacaaaaacagcatattgacgctgggaaag

SEQ ID NO: 9 - DNA, 34 bp, Artificial Sequence
  Synthetic DENV 1
  misc_feature (1)..(34): contains 3 FP modification; see specification as filed for detailed description of modifications
aacagcatattgacgctgggagagaccagagatc

SEQ ID NO: 10 - DNA, 49 bp, Artificial Sequence
  Synthetic DenMPExoP9FAM
  misc_feature (1)..(49): contains FAM, dSpacer, BHQ1 modifications; see specification as filed for detailed description of modifications
ccattttctggcgttctgtgcctggaatgatgttgtgagacagcaggat

SEQ ID NO: 11 - DNA, 36 bp, Artificial Sequence
  Synthetic DEV 1
  misc_feature (1)..(36): contains 3 RP modification; see specification as filed for detailed description of modifications
cacagaacgccagaaaatggaatggtgctgttgaat

SEQ ID NO: 12 - DNA, 29 bp, Artificial Sequence
  Synthetic DEV 4 RP
tcaatccaggcacagagcgccgcaagatg

SEQ ID NO: 13 - DNA, 32 bp, Artificial Sequence
  Synthetic WNV_5UTR_F3
tcgcctgtgtgagctgacaaacttagtagtgt

SEQ ID NO: 14 - DNA, 32 bp, Artificial Sequence
  Synthetic WNV_5UTR_R1
ggcattccgcgttttagcatattgacagcccg

SEQ ID NO: 15 - DNA, 46 bp, Artificial Sequence
  Synthetic WNV_5UTR_P3
  misc_feature (1)..(46): contains FAM, dSpacer, BHQ1 modifications; see specification as filed for detailed description of modifications
gagctgtttcttagcacgaagatctcgatgttaagaaaccaggagg

SEQ ID NO: 16 - DNA, 32 bp, Artificial Sequence
  Synthetic Zika_NS3_F2
gtcatacagctcagcagaaagacttttgagac

SEQ ID NO: 17 - DNA, 32 bp, Artificial Sequence
  Synthetic Zika_NS3_R4
aggcatctcctggaatctatgacacggtcagc

SEQ ID NO: 18 - DNA, 46 bp, Artificial Sequence
  Synthetic Zika_NS3_P3
  misc_feature (1)..(46): contains FAM, THF, BHQ1 modifications; see specification as filed for detailed description of modifications
aagagtgggactttgtcgtgacaactgacattcagagatgggcgcc

SEQ ID NO: 19 - DNA, 32 bp, Artificial Sequence
  Synthetic Babesia A2 FP4
cagcatggaataatgaagtaggactttggttc

SEQ ID NO: 20 - DNA, 31 bp, Artificial Sequence
  Synthetic Babesia A2 RP13
tccttggcaaatgctttcgcagtagttcgtc

SEQ ID NO: 21 - DNA, 45 bp, Artificial Sequence
  Synthetic Babesia A2 P2
  misc_feature (1)..(45): contains FAM, dSpacer, BHQ1 modifications; see specification as filed for detailed description of modifications
gcattcgtatttaactgtcagaggtgaaatcttagatttgttaaa

SEQ ID NO: 22 - DNA, 32 bp, Artificial Sequence
  Synthetic Malaria A1 FP4
cgagtttctgacctatcagcttttgatgttag

SEQ ID NO: 23 - DNA, 32 bp, Artificial Sequence
  Synthetic Malaria A1 RP2
tatttcttgtcactacctctcttctttagaat

SEQ ID NO: 24 - DNA, 45 bp, Artificial Sequence
  Synthetic Malaria A1 P6
  misc_feature (1)..(45): contains FAM, dSpacer, BHQ1 modifications; see specification as filed for detailed description of modifications
aattagagttcgattccggagagggagcctataaatagctaccac

SEQ ID NO: 25 - DNA, 29 bp, Artificial Sequence
  Synthetic HIV-1 INT FP1
attccctacaatccccaaagtcaaggagt

SEQ ID NO: 26 - DNA, 27 bp, Artificial Sequence
  Synthetic HIV-1 INT RP7
tctttcccctgcactgtaccccccaat

SEQ ID NO: 27 - DNA, 48 bp, Artificial Sequence
  Synthetic HIV-1 INT P4
  misc_feature (1)..(48): contains FAM, dSpacer, BHQ1, Spacer-C3 modifications; see specification as filed for detailed description of modifications
acagcagtacaaatggcagtattcattcacaatttaaaagaaaagggg

SEQ ID NO: 28 - DNA, 28 bp, Artificial Sequence
  Synthetic HIV-1 LTR FP1
aagcctcaataaagcttgccttgagtgc

SEQ ID NO: 29 - DNA, 29 bp, Artificial Sequence
  Synthetic HIV-1 LTR RP3
cttcagcaagccgagtcctgcgtcgagag

SEQ ID NO: 30 - DNA, 46 bp, Artificial Sequence
  Synthetic HIV-1 LTR P1
  misc_feature (1)..(46): contains FAM, dSpacer, BHQ1, Spacer-C3 modifications; see specification as filed for detailed description of modifications
gtgtgtgcccgtctgttgtgtgactctggtactagagatccctcag

SEQ ID NO: 31 - DNA, 46 bp, Artificial Sequence
  Synthetic HBV-P5
  misc_feature (1)..(46): contains Quasar 705, dSpacer, BHQ2, SpacerC3 modifications; see specification as filed for detailed description of modifications
agcgctggatgtgtctgcggcgttttatcatttcctcttcatcctg

SEQ ID NO: 32 - DNA, 32 bp, Artificial Sequence
  Synthetic HBV-FPe
ctagactcgtggtggacttctctcaattttct

SEQ ID NO: 33 - DNA, 51 bp, Artificial Sequence
  Synthetic HBV P2
  misc_feature (1)..(51): contains FAM, dSpacer, BHQ1, Spacer-C3 modifications; see specification as filed for detailed description of modifications
cttggccaaaattcgcagtccccaacctccaatcctcaccaacctcctgtc

SEQ ID NO: 34 - DNA, 33 bp, Artificial Sequence
  Synthetic HCV FP6a
caatgcctggagatttgggcgtgcccccgcaag

SEQ ID NO: 35 - DNA, 48 bp, Artificial Sequence
  Synthetic VP1- P3
  misc_feature (1)..(48): contains Q670, dSpacer, BHQ2, C3-Spacer modifications; see specification as filed for detailed description of modifications
cagaacctagaggagaagatgcagtattatctatgaagacttacacaa

SEQ ID NO: 36 - DNA, 32 bp, Artificial Sequence
  Synthetic VP1 -FP5
tatctgaccacccccatgccttatcatccagt

SEQ ID NO: 37 - DNA, 32 bp, Artificial Sequence
  Synthetic VP1-RP4
tggctatacctaaagtcatgaatccttgcagc

SEQ ID NO: 38 - DNA, 32 bp, Artificial Sequence
  Synthetic VP1 -FP1
attttcaaagtcatggacagttatctgaccac

SEQ ID NO: 39 - DNA, 32 bp, Artificial Sequence
  Synthetic VP1 -FP2
caaagtcatggacagttatctgaccaccccca

SEQ ID NO: 40 - DNA, 32 bp, Artificial Sequence
  Synthetic VP1 -FP3
ccatggacagttatctgaccacccccatgcct

SEQ ID NO: 41 - DNA, 32 bp, Artificial Sequence
  Synthetic VP1 -FP4
gacagttatctgaccacccccatgccttatca

SEQ ID NO: 42 - DNA, 31 bp, Artificial Sequence
  Synthetic VP1 -RP1
gtcatgaatccttgcagcactgtcaacagca

SEQ ID NO: 43 - DNA, 32 bp, Artificial Sequence
  Synthetic VP1 -RP2
tacctaaagtcatgaatccttgcagcactgtc

SEQ ID NO: 44 - DNA, 32 bp, Artificial Sequence
  Synthetic VP1 -RP3
gctatacctaaagtcatgaatccttgcagcac

SEQ ID NO: 45 - DNA, 48 bp, Artificial Sequence
  Synthetic VP1 -P2
  misc_feature (1)..(48): contains BHQ1, dSpacer, C3-Spacer modifications; see specification as filed for detailed description of modifications
cagtcatgcagaacctagaggagaagatgcagtttatctagtgaagac

SEQ ID NO: 46 - DNA, 50 bp, Artificial Sequence
  Synthetic VP1 -P7
  misc_feature (1)..(50): contains BHQ1, dSpacer, C3-Spacer modifications; see specification as filed for detailed description of modifications
ggcaagttagcgtacaactacccggtactaactatttgggcctggcaatg

SEQ ID NO: 47 - DNA, 51 bp, Artificial Sequence
  Synthetic NS1- P5
  misc_feature (1)..(51): contains BHQ1, dSpacer, C3-Spacer modifications; see specification as filed for detailed description of modifications
tggtgaaagctctgaagaactcagtgaaagcagctttttaacctcatcacc

SEQ ID NO: 48 - DNA, 32 bp, Artificial Sequence
  Synthetic NS1-FP6
agtggtggtgaaagctctgaagaactcagtga

SEQ ID NO: 49 - DNA, 32 bp, Artificial Sequence
  Synthetic NS1-RP4
gaggaaactgggcttccgacaaatgattctcc

SEQ ID NO: 50 - DNA, 32 bp, Artificial Sequence
  Synthetic NS1 -FP3
agtatcagcagcagtggtggtgaaagctctga

SEQ ID NO: 51 - DNA, 32 bp, Artificial Sequence
  Synthetic NS1 -FP5
gcagcagtggtggtgaaagctctgaagaactc

SEQ ID NO: 52 - DNA, 32 bp, Artificial Sequence
  Synthetic NS1-RP6
cttagccaattggctatacctaaagtcatgaa

SEQ ID NO: 53 - DNA, 50 bp, Artificial Sequence
  Synthetic NS1-P3
  misc_feature (1)..(50): contains BHQ1, dSpacer, C3-Spacer modifications; see specification as filed for detailed description of modifications
tgaaagctctgaagaactcagtgaaagcagctttttaacctcatcacccc

SEQ ID NO: 54 - DNA, 32 bp, Artificial Sequence; identical to SEQ ID NO: 13
  Synthetic WNV_5UTR_F3

SEQ ID NO: 55 - DNA, 32 bp, Artificial Sequence; identical to SEQ ID NO: 14
  Synthetic WNV_5UTR_R1

SEQ ID NO: 56 - DNA, 46 bp, Artificial Sequence; identical to SEQ ID NO: 15
  Synthetic WNV_5UTR_P3
  misc_feature (1)..(46): contains FAM, dSpacer, BHQ1 modifications; see specification as filed for detailed description of modifications

SEQ ID NO: 57 - DNA, 32 bp, Artificial Sequence; identical to SEQ ID NO: 16
  Synthetic Zika_NS3_F2

SEQ ID NO: 58 - DNA, 32 bp, Artificial Sequence; identical to SEQ ID NO: 17
  Synthetic Zika_NS3_R4

SEQ ID NO: 59 - DNA, 47 bp, Artificial Sequence
  Synthetic Zika_NS3_P3
  misc_feature (1)..(47): contains FAM, THF, BHQ1 modifications; see specification as filed for detailed description of modifications
aagagtgggactttgtcgtgacaactgacatttcagagatgggcgcc

SEQ ID NO: 60 - DNA, 30 bp, Artificial Sequence; identical to SEQ ID NO: 8
  Synthetic DENV 4 FP

SEQ ID NO: 61 - DNA, 34 bp, Artificial Sequence; identical to SEQ ID NO: 9
  Synthetic DENV 1-3 FP

SEQ ID NO: 62 - DNA, 49 bp, Artificial Sequence; identical to SEQ ID NO: 10
  Synthetic DenMPExoP9FAM
  misc_feature (1)..(49): contains FAM, dSpacer, BHQ1, Spacer-C3 modifications; see specification as filed for detailed description of modifications

SEQ ID NO: 63 - DNA, 36 bp, Artificial Sequence; identical to SEQ ID NO: 11
  Synthetic DEV 1-3 RP

SEQ ID NO: 64 - DNA, 29 bp, Artificial Sequence
  Synthetic DEV 4 RP
tcaatccaggcacagagcgccgcaagatg

SEQ ID NO: 65 - DNA, 32 bp, Artificial Sequence; identical to SEQ ID NO: 4
  Synthetic CHIKV_E1 _F7

SEQ ID NO: 66 - DNA, 32 bp, Artificial Sequence; identical to SEQ ID NO: 5
  Synthetic CHIKV_E1_R7

SEQ ID NO: 67 - DNA, 32 bp, Artificial Sequence; identical to SEQ ID NO: 6
  Synthetic CHIKV_E1_R8

SEQ ID NO: 68 - DNA, 49 bp, Artificial Sequence; identical to SEQ ID NO: 7
  Synthetic CHIKV_E1_P3
  misc_feature (1)..(49): contains FAM, THF, BHQ1 modifications; see specification as filed for detailed description of modifications

SEQ ID NO: 69 - DNA, 32 bp, Artificial Sequence; identical to SEQ ID NO: 19
  Synthetic Babesia A2 FP4

SEQ ID NO: 70 - DNA, 31 bp, Artificial Sequence; identical to SEQ ID NO: 20
  Synthetic Babesia A2 RP13

SEQ ID NO: 71 - DNA, 45 bp, Artificial Sequence; identical to SEQ ID NO: 21
  Synthetic Babesia A2 P2
  misc_feature (1)..(45): contains FAM, dSpacer, BHQ1, Spacer-C3 modifications; see specification as filed for detailed description of modifications

SEQ ID NO: 72 - DNA, 32 bp, Artificial Sequence; identical to SEQ ID NO: 22
  Synthetic Malaria A1 FP4

SEQ ID NO: 73 - DNA, 32 bp, Artificial Sequence; identical to SEQ ID NO: 23
  Synthetic Malaria A1 RP2

SEQ ID NO: 74 - DNA, 44 bp, Artificial Sequence
  Synthetic Malaria A1 P6
  misc_feature (1)..(44): contains FAM, dSpacer, BHQ1 modifications; see specification as filed for detailed description of modifications
aattagagttcgattccggagagggagcctaaaatagctaccac

SEQ ID NO: 75 - DNA, 29 bp, Artificial Sequence
  Synthetic HEV A1 FP1
ccatcgcctatgccttatgtcccttaccc

SEQ ID NO: 76 - DNA, 29 bp, Artificial Sequence
  Synthetic HEV A1 FP2
atgccttatgtcccttaccctcgttcaac

SEQ ID NO: 77 - DNA, 29 bp, Artificial Sequence
  Synthetic HEV A1 FP3
tatgtcccttaccctcgttcaaccgaggt

SEQ ID NO: 78 - DNA, 31 bp, Artificial Sequence
  Synthetic HEV A2 FP1
cgtctggccgccgtcgtgggcggcgcagcgg

SEQ ID NO: 79 - DNA, 30 bp, Artificial Sequence
  Synthetic HEV A2 FP2
tggccgccgtcgtgggcggcgcagcggcgg

SEQ ID NO: 80 - DNA, 30 bp, Artificial Sequence
  Synthetic HEV A2 FP3
cgccgtcgtgggcggcgcagcggcggtgcc

SEQ ID NO: 81 - DNA, 30 bp, Artificial Sequence
  Synthetic HEV A2 FP4
cgtcgtgggcggcgcagcggcggtgccggc

SEQ ID NO: 82 - DNA, 26 bp, Artificial Sequence
  Synthetic HEV A3 FP1
ccgacagaattgatttcgtcggctgg

SEQ ID NO: 83 - DNA, 28 bp, Artificial Sequence
  Synthetic HEV A3 FP2
cagaattgatttcgtcggctggtggtca

SEQ ID NO: 84 - DNA, 30 bp, Artificial Sequence
  Synthetic HEV A3 FP3
attgatttcgtcggctggtggtcagctgtt

SEQ ID NO: 85 - DNA, 31 bp, Artificial Sequence
  Synthetic HEV A3 FP4
tttcgtcggctggtggtcagctgttttactc

SEQ ID NO: 86 - DNA, 29 bp, Artificial Sequence
  Synthetic HEV A1 RP1
aatgtagacttagtagagcaggctgatgg

SEQ ID NO: 87 - DNA, 29 bp, Artificial Sequence
  Synthetic HEV A1 RP2
acagcatgaaatgtagacttagtagagca

SEQ ID NO: 88 - DNA, 26 bp, Artificial Sequence
  Synthetic HEV A1 RP3
tcccagatatgcacagggacagcatg

SEQ ID NO: 89 - DNA, 28 bp, Artificial Sequence
  Synthetic HEV A2 RP1
aaggggttggttggatgaatatagggga

SEQ ID NO: 90 - DNA, 28 bp, Artificial Sequence
  Synthetic HEV A2 RP2
gcgaaggggttggttggatgaatatagg

SEQ ID NO: 91 - DNA, 30 bp, Artificial Sequence
  Synthetic HEV A2 RP3
tcggaggcgaaggggttggttggatgaata

SEQ ID NO: 92 - DNA, 30 bp, Artificial Sequence
  Synthetic HEV A3 RP1
atacccttatcctgctgagcattctcgaca

SEQ ID NO: 93 - DNA, 32 bp, Artificial Sequence
  Synthetic HEV A3 RP2
atagcaatacccttatcctgctgagcattctc

SEQ ID NO: 94 - DNA, 29 bp, Artificial Sequence
  Synthetic HEV A3 RP3
gactcaccaaggtctatatcatgtgggat

SEQ ID NO: 95 - DNA, 32 bp, Artificial Sequence
  Synthetic HEV A3 RP4
acacgggactcaccaaggtctatatcatgtgg

SEQ ID NO: 96 - DNA, 45 bp, Artificial Sequence
  Synthetic HEV A1 P1
tgtcccttaccctcgttcaaccgaggtgtatgttcggtccatatt

SEQ ID NO: 97 - DNA, 47 bp, Artificial Sequence
  Synthetic HEV A1 P2
cctcgttcaaccgaggtgtatgttcggtccatatttggccctggtgg

SEQ ID NO: 98 - DNA, 47 bp, Artificial Sequence
  Synthetic HEV A1 P3
tccatatttggccctggtggttccccatccctgtttccatcagcctg

SEQ ID NO: 99 - DNA, 46 bp, Artificial Sequence
  Synthetic HEV A2 P1
ccggcggtggtttctggggtgaccgggttgattctcagcccttcgc

SEQ ID NO: 100 - DNA, 49 bp, Artificial Sequence
  Synthetic HEV A2 P2
gtggtttctggggtgaccgggttgattctcagcccttcgccctccccta

SEQ ID NO: 101 - DNA, 44 bp, Artificial Sequence
  Synthetic HEV A3 P1
tactcccgccccgtcgtctcagccaatggcgagccgactgtgaa

SEQ ID NO: 102 - DNA, 44 bp, Artificial Sequence
  Synthetic HEV A3 P2
gtcgtctcagccaatggcgagccgactgtgaagctatacacatc

SEQ ID NO: 103 - DNA, 30 bp, Artificial Sequence
  Synthetic IC FP1
cagaaactacagcagagttggcagcttcac

SEQ ID NO: 104 - DNA, 31 bp, Artificial Sequence
  Synthetic IC RP2
gtctggcctttcagcaagtttccaacaaaca

SEQ ID NO: 105 - DNA, 46 bp, Artificial Sequence
  Synthetic IC P1
  misc_feature (1)..(46): contains CAL Fluor Orange 560, dSpacer, BHQ-1 modifications; see specification as filed for detailed description of modifications
agctgacgagttcatgagggcaggccgctagatggatggcttccaa

SEQ ID NO: 106 - DNA, 32 bp, Artificial Sequence
  Synthetic COVID-19 FP1
taacatgcttagaattatggcctcacttgttc

SEQ ID NO: 107 - DNA, 32 bp, Artificial Sequence
  Synthetic COVID-19 FP2
aaagaagaaggctgatgaaactcaagccttac

SEQ ID NO: 108 - DNA, 32 bp, Artificial Sequence
  Synthetic COVID-19 FP3
gctgatgaaactcaagccttaccgcagagaca

SEQ ID NO: 109 - DNA, 31 bp, Artificial Sequence
  Synthetic COVID-19 RP1
tgaccatttcactcaatacttgagcacactc

SEQ ID NO: 110 - DNA, 32 bp, Artificial Sequence
  Synthetic COVID-19 RP2
actgctcatggattgttgcaattgtttggaga

SEQ ID NO: 111 - DNA, 32 bp, Artificial Sequence
  Synthetic COVID-19 RP3
ttgagtcagcactgctcatggattgttgcaat

SEQ ID NO: 112 - DNA, 47 bp, Artificial Sequence
  Synthetic COVID-19 P1
  misc_feature (1)..(47): contains FAM, dSpacer, BHQ1, Spacer-C3 modifications; see specification as filed for detailed description of modifications
cttgctcgcaaacatacaacgtgttgtagctgtcacaccgtttctat

SEQ ID NO: 113 - DNA, 49 bp, Artificial Sequence
  Synthetic COVID-19 P2
  misc_feature (1)..(49): contains FAM, dSpacer, BHQ1, Spacer-C3 modifications; see specification as filed for detailed description of modifications
agccttaccgcagagacagaagaaacagcaaacttgactcttcttcctg

SEQ ID NO: 114 - DNA, 48 bp, Artificial Sequence
  Synthetic COVID-19 P3
  misc_feature (1)..(48): contains FAM, dSpacer, BHQ1, Spacer-C3 modifications; see specification as filed for detailed description of modifications
cgcagagacagaagaaacagcaaactgtgactctcttcctgctgcaga

SEQ ID NO: 115 - DNA, 49 bp, Artificial Sequence
  Synthetic COVID-19 P4
  misc_feature (1)..(49): contains FAM, dSpacer, BHQ1, Spacer-C3 modifications; see specification as filed for detailed description of modifications
agacagaagaaacagcaaactgtgactcttcttctgctgcagatttgga

SEQ ID NO: 116 - DNA, 217 bp, Artificial Sequence
  Synthetic
tatctgaccacccccatgccttatcatccagtagcagtcatgcagaacctagaggagaagatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagcca

SEQ ID NO: 117 - DNA, 149 bp, Artificial Sequence
  Synthetic
agtggtggtgaaagctctgaagaactcagtgaaagcagcttttttaacctcatcaccccaggcgcctggaacactgaaaccccgcgctctagtacgcccatccccgggaccagttcaggagaatcatttgtcggaagcccagtttcctc

SEQ ID NO: 118 - DNA, 236 bp, Artificial Sequence
  Synthetic
attttcaaagtcatggacagttatctgaccacccccatgccttatcatccagtagcagtcatgcagaacctagaggagaagatgcagtattatctagtgaagacttacacaagcctgggcaagttagcgtacaactacccggtactaactatgttgggcctggcaatgagctacaagctgggcccccgcaaagtgctgttgacagtgctgcaaggattcatgactttaggtatagc

SEQ ID NO: 119 - DNA, 173 bp, Artificial Sequence
  Synthetic
agtatcagcagcagtggtggtgaaagctctgaagaactcagtgaaagcagcttttttaacctcatcaccccaggcgcctggaacactgaaaccccgcgctctagtacgcccatccccgggaccagttcaggagaatcatttgtcggaagcccagtttcctccgaagttgtagc

SEQ ID NO: 120 - DNA, 182 bp, Artificial Sequence
  Synthetic
tccatgattagcatggagctgtaggagtctaaattggggacgcagatgtttgggacgtcaccttgcagtgttaacttggctttcatgaacctctttgatctttcacaagaggtaggctacgggtgaaacctcttaggctaatacttctatgaagagatgccttggatagggtaacagcggcg

SEQ ID NO: 121 - DNA, 195 bp, Artificial Sequence
  Synthetic
tccatgattagcatggagctgtaggagtctaaattggggacgcagatgtttgggacgtcaccttgcagtgttaacttggctttcatgaacctctttgatctttcacaagaggtaggctacgggtgaaacctcttaggctaatacttctatgaagagatgccttggatagggtaacagcggcggatattggtgagt

SEQ ID NO: 122 - DNA, 149 bp, Artificial Sequence
  Synthetic
taatgaagtaggactttggttctattttgttggttattgagccagagtaatggttaataggagcagttgggggcattcgtatttaactgtcagaggtgaaattcttagatttgttaaagacgaactactgcgaaagcatttgccaagga

SEQ ID NO: 123 - DNA, 175 bp, Artificial Sequence
  Synthetic
taatgaagtaggactttggttctattttgttggttattgagccagagtaatggttaataggagcagttgggggcattcgtatttaactgtcagaggtgaaattcttagatttgttaaagacgaactactgcgaaagcatttgccaaggatgttttcattaatcaagaacgaaagt

SEQ ID NO: 124 - DNA, 181 bp, Artificial Sequence
  Synthetic
taatgaagtaggactttggttctattttgttggttattgagccagagtaatggttaataggagcagttgggggcattcgtatttaactgtcagaggtgaaattcttagatttgttaaagacgaactactgcgaaagcatttgccaaggatgttttcattaatcaagaacgaaagttagggg